(19) United States
(12) Patent Application Publication
(10) Pub. No.: US 2024/0327382 A1
(43) Pub. Date: Oct. 3, 2024

(54) PHARMACEUTICAL COMPOUNDS AS INHIBITORS OF UBIQUITIN SPECIFIC PROTEASE 19

(71) Applicant: ALMAC DISCOVERY LIMITED, Craigavon (GB)

(72) Inventors: James Samuel Shane ROUNTREE, Craigavon (GB); Steven Kristopher WHITEHEAD, Craigavon (GB); Steven David SHEPHERD, Craigavon (GB); Matthew Duncan HELM, Craigavon (GB); Frank BURKAMP, Craigavon (GB); Colin O'DOWD, Craigavon (GB); Timothy HARRISON, Craigavon (GB)

(21) Appl. No.: 18/551,511

(22) PCT Filed: Mar. 24, 2022
(86) PCT No.: PCT/EP2022/057820

§ 371 Date: Sep. 20, 2023

(30) Foreign Application Priority Data
Mar. 24, 2021 (GB) 2104097.7

(51) Int. Cl. C07D 401/14; A61K 31/4545; A61K 31/496; A61K 31/506; A61K 31/517; A61K 31/519; A61K 31/5377; A61P 3/04; A61P 3/10; A61P 21/00; C07D 401/06; C07D 405/14; C07D 409/14; C07D 413/14; C07D 471/04; C07D 487/04

(52) U.S. Cl. CPC C07D 401/14; A61K 31/4545; A61K 31/496; A61K 31/506; A61K 31/517; A61K 31/519; A61K 31/5377; A61P 3/04; A61P 3/10; A61P 21/00; C07D 401/06; C07D 405/14; C07D 409/14; C07D 413/14; C07D 471/04; C07D 487/04

(57) ABSTRACT

The present invention relates to compounds of Formula (I) that are useful as inhibitors of the activity of the ubiquitin specific protease USP19. The present invention also relates to pharmaceutical compositions comprising these compounds and to methods of using these compounds in therapy.

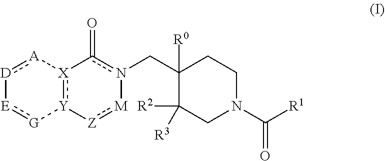

### FIELD OF INVENTION

The present invention concerns inhibitors of ubiquitin specific protease 19 (USP19) and methods of use thereof.

### BACKGROUND

Over the past decade, protein ubiquitination has emerged as an important post-translational modification with roles in a plethora of cellular processes including amongst others proteolysis, gene expression, DNA repair, immune response, metabolism or cell cycle regulation. Dysregulation of the Ubiquitin Proteasome System (UPS) has also been implicated in the pathogenesis of multiple human diseases including but not limited to cancer (Hoeller D. et al., Nat. Rev. Cancer (2006), 6, 776-788), viral infection (Gao et al., J. Physiol., Pharmacol. (2006), 84, 5-14), metabolic or neurodegenerative disorders (Loosdregt J. et al., Immunity (2013), 39, 259-271; Rubinsztein D., et al., Nature (2006), 443, 780-786) as well as immune and inflammatory-related medical conditions (Wang J. et al., J. Cell Immunol. (2006), 3, 255-261; Corn J. et al., Nat. Struct. & Mol. Biol. (2014), 21, 297-300; Nicholson B. et al., Cell Biochem. Biophys. (2013), 60, 61-68).

The approval and clinical success of the proteasome inhibitors Velcade® (bortezomib) or Kyprolis® (carfilzomib) for the treatment of mantel cell lymphoma (AML) and multiple myeloma (MM) have validated the UPS as a cancer target amenable for pharmacological intervention. Although effective, their clinical utility has however been severely limited due to poor selectivity and acute toxicity issues. By inhibiting the 26S proteasome, the current proteasome inhibitors indiscriminately impair proteolysis in both cancer and normal cells and are characterised by a low therapeutic index. To circumvent this issue, a promising alternative approach may be to target the UPS upstream of the proteasome. Interfering with the ubiquitin (Ub) conjugation/deconjugation machinery, for instance at the level of the Ubiquitin Specific Proteases (USPs), would allow for the development of improved therapeutics with enhanced specificity and reduced toxicity profiles.

USPs are the largest subfamily of the deubiquitinating enzymes (DUBs) family with over 60 family members reported to date (Komander D. et al., Nat. Rev. Mol. (2009), 10, 550-563; Clague M. et al., Physiol. Rev. (2013), 93, 1289-1315). USPs are typically cysteine proteases that catalyse the removal of Ub from specific target substrates thus preventing their induced degradation by the proteasome, or regulating their activation and/or subcellular localization (Colland F. et al., Biochimie (2008), 90, 270-283; Nicholson B. et al., Cell Biochem. Biophys. (2013), 60, 61-68). It is now well established that USPs regulate the stability and activation of numerous proteins involved in the pathogenesis of human diseases including both oncogenes and tumor suppressors. As such, USPs represent an emerging and attractive target class for pharmacological intervention.

Amongst all USPs, USP19 is an important member due to its association with a number of important pathways with implications for pathological conditions including but not restricted to cancer, neurodegeneration and degenerative diseases as well as antiviral immune response. USP19 expresses as multiple isoforms varying in length from 71.09 kDa (isoform 2) to 156.03 kDa (isoform 5) with the canonical sequence (isoform 1) of 145.65 kDa in size (uniprot.org). The cellular localisation of USP19 may be cytosolic or bound to the endoplasmic reticulum (Lee J. et al., J. Biol. Chem. (2014), 289, 3510-3507; Lee J. et al., Nat. Cell Biol. (2016), 18, 765-776). Localised to the endoplasmic reticulum, USP19 is a key component of the endoplasmic reticulum-associated degradation (ERAD) pathway (Hassink B. et al., EMBO J. (2009), 10, 755-761; Lee J. et al., J. Biol. Chem. (2014), 289, 3510-3507; Lee J. et al., Nat. Cell Biol. (2016), 18, 765-776). In particular, USP19 is involved in the latter steps of the protein quality-control machinery rescuing ERAD substrates that have been retro-translocated to the cytosol. USP19 has also been demonstrated to regulate the stability of the E3 ligases MARCH6 and HRD1 (Nakamura N. et al., Exp. Cell Res. (2014), 328, 207-216; Harada K. et al., Int. J. Mol. Sci. (2016), 17, E1829). In addition, USP19 has recently been implicated in the stabilisation of multiple and potentially important protein substrates. For instance, USP19 interacts with SIAH proteins to rescue HIF1α from degradation under hypoxic conditions (Altun M. et al., J. Biol. Chem. (2012), 287, 1962-1969; Velasco K. et al., Biochem. Biophys. Res. Commun. (2013), 433, 390-395). USP19 also stabilises the KPC1 ubiquitin ligase which is involved in the regulation of the p27^{Kip1} cyclin-dependent kinase inhibitor (Lu Y. et al., Mol. Cell Biol. (2009), 29, 547-558). Knock-out of USP19 by RNAi leads to p27^{Kip1} accumulation and inhibition of cell proliferation (Lu L. et al., PLoS ONE (2011), 6, e15936). USP19 was also found to interact with the inhibitors of apoptosis (IAPs) including c-IAP1 and c-IAP2 (Mei Y. et al., J. Biol. Chem. (2011), 286, 35380-35387). Knockdown of USP19 decreases the total levels of these c-IAPs whilst overexpression increases the levels of both BIRC2/cIAP1 and BIRC3/cIAP2. Knockdown of USP19 also enhances TNFα-induced caspase activation and apoptosis in a BIRC2/c-IAP1 and BIRC3/c-IAP2 dependent manner. In addition to some direct involvement in regulating hypoxia response and ER stress, USP19 has also been implicated recently as a positive regulator of autophagy and negative regulator of type I interferon signalling (IFN, antiviral immune response) by deubiquitinating Beclin-1. USP19 was found to stabilise Beclin-1 at the post-translational level by removing the K11-linked ubiquitin chains of Beclin-1 at Lysine 437 (Jin S. et al., EMBO J. (2016), 35, 866-880). USP19 negatively regulates type I IFN signalling pathway, by blocking RIG-1-MAVS interaction in a Beclin-1 dependent manner. Depletion of either USP19 or Beclin-1 inhibits autophagic flux and promotes type I IFN signalling as well as cellular antiviral immunity (Jin S. et al., EMBO J. (2016), 35, 866-880; Cui J. et al., Autophagy (2016), 12, 1210-1211). Recent findings also indicate USP19 may negatively affect the cellular antiviral type I IFN signalling by regulating the TRAF3 substrate (Gu Z. et al., Future Microbiol. (2017), 12, 767-779). USP19 has also been recently implicated in the Wnt signalling pathway by stabilising the coreceptor LRP6 (Perrody E. et al., eLife (2016), 5, e19083) and in the DNA repair processes, most particularly chromosomal stability and integrity, by regulating the HDAC1 and HDAC2 proteins (Wu M. et al., Oncotarget (2017), 8, 2197-2208).

In addition to cancer and associated conditions, USP19 has been linked in gene knock out studies to muscle-wasting syndromes and other skeletal muscle atrophy disorders (Wing S., Int. J. Biochem. Cell Biol. (2013), 45, 2130-2135; Wing S. et al., Int. J. Biochem. Cell Biol. (2016), 79, 426-468; Wiles B. et al., Mol. Biol. Cell (2015), 26, 913-923; Combaret L. et al., Am. J. Physiol. Endocrinol. Metab. (2005), 288, E693-700, each of which is incorporated herein by reference). Muscle wasting associated with conditions such as cachexia is known to impair quality of life and response to therapy, which increase morbidity and mortality of cancer patients. Muscle wasting is also associated with other serious illnesses such as HIV/AIDS, heart failure, rheumatoid arthritis and chronic obstructive pulmonary disease (Wiles B. et al., Mol. Biol. Cell (2015), 26, 913-923). Muscle wasting is also a prominent feature of aging.

Beyond the above pathological conditions, USP19 may also have implications in the pathogenesis of degenerative diseases including but not restricted to Parkinson's disease and other prion-like transmission disorders by regulating important substrates such as α-synuclein or polyglutamine-containing proteins, Ataxin3, Huntington (He W. et al., PLoS ONE (2016), 11, e0147515; Bieri G. et al., Neurobiol Dis. (2018), 109B, 219-225). The regulation of coronin 2 A (CORO2 A) through the activity of USP19 has been shown to affect the transcriptional repression activity of the retinoic acid receptor (RAR), suggesting that USP19 may also be involved in the regulation of RAR-mediated adipogenesis (Lim K. et al., Oncotarget (2016), 7, 34759-34772).

The established and ever growing connections between USP19 and numerous proteins involved in human pathologies indicate that small molecule inhibitors of USP19 may have broad therapeutic applications beneficial to human health. Insofar as is known however, no inhibitors targeting USP19 have been reported and the identification of such inhibitors with drug-like potential therefore remains of prime importance and high priority.

### SUMMARY OF INVENTION

In a first aspect is provided a compound of formula (I):

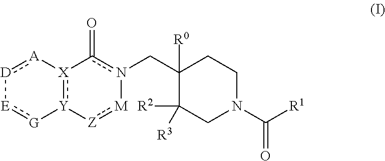

wherein
R⁰ is H, NH2, F, or OCH3;
R¹ is optionally substituted C1-C6 alkyl, ethylcyclopropyl, ethylcyclobutyl, optionally substituted C5-C8 aryl, optionally substituted C3-C8 heteroaryl, optionally substituted C3-C8 heterocycloalkyl, NR^{a}R^{b}, NR^{a}CH2R^{b}, OR^{a}, or OCH2R^{a}
   wherein R^{a} and R^{b} are independently selected from H, C1-C6 alkyl, CF3, optionally substituted C3-C6 cycloalkyl, optionally substituted C5-C8 aryl, optionally substituted C6-C9 arylalkyl, and optionally substituted C2-C8 heteroaryl, and wherein when R¹ is NR^{a}CH2R^{b}, the methylene group may be optionally substituted with CF3,
   or R¹ is NR^{a}R^{b} and R^{a} and R^{b} together form an optionally substituted C2-C9 heterocycle together with the N to which they are attached;
R² and R³ are independently selected from H, and C1-C6 alkyl, or together form a C3-C6 cycloalkyl or C3-C6 heterocycloalkyl with the carbon to which they attached;
X is absent, C, CR^{4a}, CR^{4a}R^{4b}, N, NR^{4a}, or C═O,
   wherein R^{4a} and R^{4b} are independently selected from H, optionally substituted C1-C6 alkyl and halo;
   or wherein R^{4a} and R^{4b} together form a C3-C6 cycloalkyl or C3-C6 heterocycloalkyl including the carbon to which they are attached;
Y is C, CR⁵, CR⁵R⁶, N, NR⁵, or O,
   wherein R⁵ and R⁶ are independently selected from H, halo, optionally substituted C1-C6 alkyl, C2-C6 alkenyl, C2-C6 alkynyl, optionally substituted C3-C6 cycloalkyl, optionally substituted C3-C6 heterocycloalkyl, optionally substituted C5-C8 aryl, optionally substituted C6-C9 arylalkyl, optionally substituted C3-C8 heteroaryl, CH2OH, NR′R″, NS(O)R′R″, SO2R′, C(O)R′, COR′, C(O)OR′, C(O)NR′R″, OR′, wherein R′ and R″ are independently selected from H, C1-C6 alkyl, C5-C8 aryl, C6-C9 arylalkyl, and C3-C8 heteroaryl, or wherein R⁵ is NR′R″ and R′ and R″ together form an optionally substituted C3-6 heterocycloalkyl including the nitrogen to which they are attached,
   or wherein R⁵ and R⁶ together form a C3-6 cycloalkyl or C3-C6 heterocycloalkyl including the carbon to which they are attached;
Z is N, NR⁷, C, CR⁷, CR⁷R⁸, or C═O,
   wherein R⁷ and R⁸ are independently selected from H, halo, cyano, oxo, optionally substituted C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, C(O)OR^{c}, CONR^{c}R^{d}, NR^{c}R^{d}, NS(O)R^{c}R^{d}, S(O)(R^{c})NR^{d}, SOR^{c}, SO2R^{c}, and SR^{c}, wherein R^{c} and R^{d} are independently H, C1-C6 alkyl, C3-C6 cycloalkyl, C5-C6 aryl, C6-C9 arylalkyl, C3-C6 heteroaryl, CN, COOH, or COCH3 or R^{c} and R^{d} together form an optionally substituted C3-C7 heterocycle together with the heteroatom to which they are attached;
   or wherein R⁷ and R⁸ together form a C3-6 cycloalkyl or C3-C6 heterocycloalkyl including the carbon to which they are attached;
M is absent, C, CR¹³ or CR¹³R¹⁴, wherein R¹³ and R¹⁴ are independently selected from H, and C1-C6 alkyl, or wherein R¹³ and R¹⁴ together form a C3-C6 cycloalkyl or C3-C6 heterocycloalkyl together with the carbon to which they are attached;
and
A is CR⁹, CHR⁹, N, NR⁹, S, or O;
D is CR⁹, CHR⁹, N or NR⁹,
G is absent, CR⁹, CHR⁹, or N,
   wherein R⁹ is independently selected from H, halo, C1-C6 alkyl, CF3, and OR*, wherein R* is selected from optionally substituted C1-C6 alkyl, optionally substituted C3-C6 cycloalkyl, and optionally substituted C3-C6 heterocycloalkyl,
E is CR¹⁰, CHR¹⁰, N, NR¹⁰, S, or O,
   wherein R¹⁰ is selected from H, halo, C1-C6 alkyl, C3-C6 cycloalkyl, C5-C8 aryl, C6-C9 arylalkyl, C4-C8 heteroaryl, SR^{x}, OR^{x}, NR^{x}R^{y}, and NS(O)R^{x}R^{y}, S(O)(R^{x})NR^{y}
   wherein R^{x} and R^{y} are independently selected from H, C1-C6 alkyl, CF3, C3-C6 cycloalkyl, C5-C8 aryl, C6-C9 arylalkyl, C4-C8 heteroaryl, COOH, amido, cyano, C2-C6 alkene, C2-C6 alkyne, or wherein R^{x} and R^{y} together form an optionally substituted C4-C6 heterocycloalkyl together with the nitrogen to which they are attached;
or A, D, E and G are all absent, optionally wherein X and M are absent, or optionally wherein Y and Z together form an optionally substituted C5-C6 aryl or C3-C6 heteroaryl fused ring or Z and M together form an optionally substituted C5-C6 aryl or C5-C6 heteroaryl fused ring;
or a compound having a structure selected from:

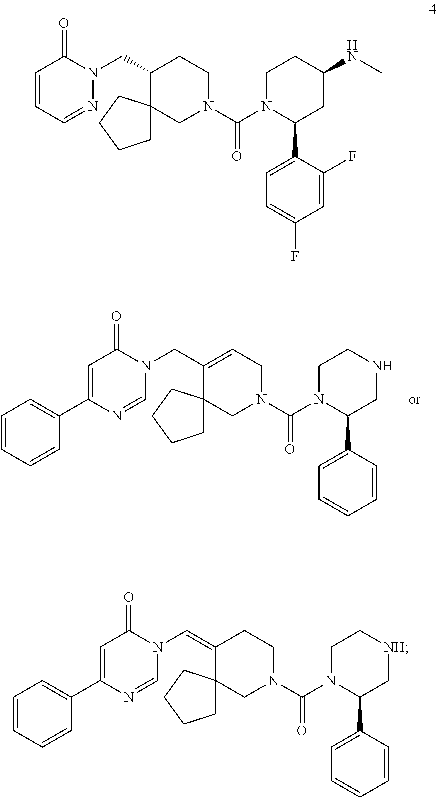 [SMILES: CN[C@@H]1CCN(C(=O)N2CC[C@@H](Cn3ncccc3=O)C3(CCCC3)C2)[C@H](c2ccc(F)cc2F)C1 ; O=C(N1CC=C(Cn2cnc(-c3ccccc3)cc2=O)C2(CCCC2)C1)N1CCNC[C@H]1c1ccccc1 ; O=C(N1CC/C(=C/n2cnc(-c3ccccc3)cc2=O)C2(CCCC2)C1)N1CCNC[C@H]1c1ccccc1]

or a stereoisomer, tautomer, hydrate, N-oxide derivative or pharmaceutically acceptable salt thereof.

In a second aspect is provided a pharmaceutical composition comprising a compound according to the first aspect, or a stereoisomer, tautomer, hydrate, N-oxide derivative or pharmaceutically acceptable salt thereof, and a pharmaceutically acceptable carrier or diluent.

USP19 has been associated with a number of diseases and conditions including (but not limited to) cancer and neoplastic conditions. Knock-out of USP19 by RNAi leads to p27^{Kip1} accumulation and inhibition of cell proliferation (Lu L. et al., PLoS ONE (2011), 6, e15936). USP19 was also found to interact with the inhibitors of apoptosis (IAPs) including c-IAP1 and c-IAP2 (Mei Y. et al., J. Biol. Chem. (2011), 286, 35380-35387). Knockdown of USP19 decreases the total levels of these c-IAPs whilst overexpression increases the levels of both BIRC2/cIAP1 and BIRC3/cIAP2. Knockdown of USP19 also enhances TNFα-induced caspase activation and apoptosis in a BIRC2/c-IAP1 and BIRC3/c-IAP2 dependent manner. USP19 has also been recently implicated in the Wnt signalling pathway by stabilising the coreceptor LRP6 (Perrody E. et al., eLife (2016), 5, e19083) and in the DNA repair processes, most particularly chromosomal stability and integrity, by regulating the HDAC1 and HDAC2 proteins (Wu M. et al., Oncotarget (2017), 8, 2197-2208).

It is further demonstrated herein that USP19 inhibitor compounds as described in relation to the first aspect exhibit cell permeability and potent target engagement in cancer cell lines. The cell permeability and target engagement in cancer cells is comparable to that observed in muscle cells. As demonstrated herein, USP19 inhibitors exhibit potent in vivo therapeutic effects on muscle wasting. Thus, by extension, since similar target engagement is seen in cancer cells, it is expected that pharmacological USP19 inhibitors will be effective at exerting therapeutic effects in cancer, due to the association of USP19 and oncogenic processes described above.

In vivo studies have also demonstrated that mice lacking the USP19 gene (USP19 KO mice) exhibited a decrease in fat mass when fed a high-fat diet (Coyne E, et al., Diabetologia (2019), 62, 136-146, which is incorporated herein by reference). USP19 KO mice also exhibited greater glucose tolerance and higher insulin sensitivity when fed a high-fat diet.

These gene knock-out studies describe a connection between USP19 and obesity, as well as USP19 and insulin sensitivity. However, before the data presented herein, no in vivo studies had been described demonstrating that pharmacological inhibitors of USP19 are an effective approach to the treatment of obesity. Similarly, before the data presented herein, no in vivo studies had demonstrated that pharmacological inhibitors of USP19 can be effective in increasing insulin sensitivity.

USP19 is also implicated in muscular atrophy, muscle-wasting syndromes and other skeletal muscle atrophy disorders (Wing S., Int. J. Biochem. Cell Biol. (2013), 45, 2130-2135; Wing S. et al., Int. J. Biochem. Cell Biol. (2016), 79, 426-468; Wiles B. et al., Mol. Biol. Cell (2015), 26, 913-923; Combaret L. et al., Am. J. Physiol. Endocrinol. Metab. (2005), 288, E693-700). This was supported for instance by studies which demonstrated that USP19-silencing induced the expression of myofibrillar proteins and promoted myogenesis (Sundaram P. et al., Am. J. Physiol. Endocrinol. Metab. (2009), 297, E1283-90; Ogawa M. et al., J. Biol. Chem. (2011), 286, 41455-41465; Ogawa M. et al., J. Endocrinol. (2015), 225, 135-145).

Knock-out studies have demonstrated that mice lacking the USP19 gene were resistant to muscle wasting in response to both glucocorticoids, a common systemic cause of muscle atrophy, as well as in response to denervation, a model of disuse atrophy (Bedard N. et al., FASEB J. (2015), 29, 3889-3898, which is incorporated herein by reference).

As set out in the accompanying Examples, it is demonstrated herein that pharmacological treatment with a USP19 inhibitor can induce therapeutic effects in a wild-type in vivo model.

In particular, it is demonstrated that USP19 inhibitors reduce fat deposition in an in vivo model, indicating that USP19 inhibitors can be an effective treatment for obesity.

Similarly, it is demonstrated herein that USP19 inhibitors can reduce loss of muscle mass in an in vivo model of muscular atrophy.

Similarly, it is demonstrated herein that USP19 inhibitors can treat the symptoms of insulin resistance, as indicated by an improved response to glucose.

The compounds according to the invention are able to selectively inhibit USP19 activity. The Examples further demonstrate that compounds which potently inhibit USP19 activity can be effective therapeutic compounds. The compounds of the invention are therefore suitable for use in methods of treatment. Indications suitable for treatment with compounds of the invention include: the treatment and prevention of cancer and neoplastic conditions; immunological and inflammatory conditions for example by promoting antiviral immune response; treatment and prevention of muscular atrophy, for example cachexia and sarcopenia; treatment and prevention of obesity; treatment and prevention of insulin resistance, for example diabetes; treatment and prevention of neurodegenerative diseases including Parkinson's disease and other prion-based disorders.

Therefore, in a further aspect, is provided a compound, or a stereoisomer, tautomer, hydrate, N-oxide derivative or pharmaceutically acceptable salt thereof, according to the first aspect, or a pharmaceutical composition according to the second aspect, for use in therapy.

In a further aspect, is provided a compound, or a stereoisomer, tautomer, hydrate, N-oxide derivative or pharmaceutically acceptable salt thereof, according to the first aspect or a pharmaceutical composition according to the second aspect for use in a method of treating or preventing cancer. In certain preferred embodiments the cancer to be treated is breast cancer or neuroblastoma.

In a further aspect is provided a compound, or a stereoisomer, tautomer, hydrate, N-oxide derivative or pharmaceutically acceptable salt thereof, according to the first aspect, or a pharmaceutical composition according to the second aspect for use in a method of treating or preventing muscular atrophy, optionally cachexia or sarcopenia.

In a further aspect is provided a compound, or a stereoisomer, tautomer, hydrate, N-oxide derivative or pharmaceutically acceptable salt thereof, according to the first aspect, or a pharmaceutical composition according to the second aspect for use in a method of treating or preventing obesity.

In a further aspect is provided a compound, or a stereoisomer, tautomer, hydrate, N-oxide derivative or pharmaceutically acceptable salt thereof, according to the first aspect, or a pharmaceutical composition according to the second aspect for use in a method of treating or preventing insulin resistance.

In a further aspect is provided a compound, or a stereoisomer, tautomer, hydrate, N-oxide derivative or pharmaceutically acceptable salt thereof, according to the first aspect, or a pharmaceutical composition according to the second aspect for use in a method of treating or preventing type II diabetes.

In a further aspect is provided a compound, or a stereoisomer, tautomer, hydrate, N-oxide derivative or pharmaceutically acceptable salt thereof, according to the first aspect, or a pharmaceutical composition according to the second aspect for use in a method of treating or preventing Parkinson's Disease.

In a further aspect is provided a method of treating cancer comprising administering to a subject an effective amount of a compound, or a stereoisomer, tautomer, hydrate, N-oxide derivative or pharmaceutically acceptable salt thereof, according to the first aspect or a pharmaceutical composition according to the second aspect.

In a further aspect is provided a method of treating muscular atrophy comprising administering to a subject an effective amount of a compound, or a stereoisomer, tautomer, hydrate, N-oxide derivative or pharmaceutically acceptable salt thereof, according to the first aspect, or a pharmaceutical composition according to the second aspect.

In a further aspect is provided a method of treating Parkinson's Disease comprising administering to a subject an effective amount of a compound, or a stereoisomer, tautomer, hydrate, N-oxide derivative or pharmaceutically acceptable salt thereof, according to the first aspect, or a pharmaceutical composition according to the second aspect.

The compounds, or stereoisomers, tautomers, hydrates, N-oxide derivatives or pharmaceutically acceptable salts thereof, may be used as monotherapy or as combination therapy with radiation and/or additional therapeutic agents.

Other preferred embodiments of the compounds provided herein appear throughout the specification and in particular in the examples. Particularly preferred are those named compounds having greater activity as tested. Compounds having higher activity are more preferred over those having lower activity.

Each aspect or embodiment as defined herein may be combined with any other aspect(s) or embodiment(s) unless clearly indicated to the contrary. In particular any feature indicated as being preferred or advantageous may be combined with any other feature or features indicated as being preferred or advantageous.

### FIGURES

FIG. 1: Effect of USP19 pharmacological inhibition on tibialis anterior mass. (A) Tibialis anterior mass (mg) from mice treated with vehicle or USP19 inhibitor compound ADC-141. Mass is given for the muscle from limb that had undergone sciatic nerve denervation (DEN) and also from the innervated limb (INN). (B) Percentage loss of tibialis anterior muscle mass as a result of denervation in vehicle and USP19 inhibitor (ADC-141) treated mice. Percentage calculated as a proportion of the mass of the muscle from the innervated limb of the same mouse. (C) Loss of tibialis anterior muscle mass (in mg) as a result of denervation in vehicle treated and USP19 inhibitor (ADC-141) treated mice. P<0.025.

FIG. 2: Effect of USP19 pharmacological inhibition on gastrocnemius muscle mass. (A) gastrocnemius muscle mass (mg) from mice treated with vehicle or USP19 inhibitor compound ADC-141. Mass is given for the muscle from limb that had undergone sciatic nerve denervation (DEN) and also from the innervated limb (INN). (B) Percentage loss of gastrocnemius muscle mass as a result of denervation in vehicle and USP19 inhibitor (ADC-141) treated mice. Percentage calculated as a proportion of the mass of the muscle from the innervated limb of the same mouse. (C) Loss of gastrocnemius muscle mass (in mg) as a result of denervation in vehicle treated and USP19 inhibitor (ADC-141) treated mice.

FIG. 3: (A) Effect of USP19 pharmacological inhibition on fat mass. The epididymal fat pad was collected from vehicle and USP19 inhibitor (ADC-141) treated mice, with USP19 inhibitor treated mice showing a significant reduction in fat mass. (B) Effect of USP19 pharmacological inhibition on liver mass. The liver was collected from vehicle and USP19 inhibitor (ADC-141) treated mice. An increase in liver mass was observed, likely due to accumulation of drug compound in the liver. (C) Percentage change in overall body weight in vehicle-treated control DIO mice. USP19 inhibitor 5 mg/kg ip BID, USP19 inhibitor 25 mg/kg ip BID, or positive control liraglutide 0.1 mg/kg sc BID (left to right bars, respectively); (D) Percentage change in overall lean mass and (E) percentage change in overall fat mass in vehicle, USP19 inhibitor 5 mg/kg, USP19 inhibitor 25 mg/kg, and liraglutide treated mice (left to right, respectively). ***p<0.001 vs vehicle

FIG. 4: Cell target engagement of USP19 inhibitor compound in breast cancer, neuroblastoma and skeletal muscle cell lines. EC₅₀ was determined by densitometry.

FIG. 5: Response to oral glucose tolerance test (OGTT) in obese mice. (A) Timeline of plasma glucose response in vehicle-treated control mice (circles), USP19 inhibitor 5 mg/kg ip BID (triangle), USP19 inhibitor 25 mg/kg ip BID (solid circle), or positive control liraglutide 0.1 mg/kg sc BID (diamond); (B) Glucose AUC (mM·hr) and (C) insulin AUC (ng·hr/ml) for vehicle, USP19 inhibitor 5 mg/kg, USP19 inhibitor 25 mg/kg, and liraglutide (left to right, respectively). **p<0.01 vs vehicle; ***p<0.001 vs vehicle.

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedence over any dictionary or extrinsic definition.

As used in the specification and the appended claims, unless specified to the contrary, the following terms have the meaning indicated:

The term “alkyl group” (alone or in combination with another term(s)) means a straight- or branched-chain saturated hydrocarbon substituent typically containing 1 to 15 carbon atoms, such as 1 to 10, 1 to 8, 1 to 6, or 1 to 4 carbon atoms. A “Cₙ alkyl” group refers to an aliphatic group containing n carbon atoms. For example, a C₁-C₁₀ alkyl group contains 1, 2, 3, 4, 5, 6, 7, 8, 9 or 10 carbon atoms. Attachment to the alkyl group occurs through a carbon atom. Examples of such substituents include methyl, ethyl, n-propyl, isopropyl, n-butyl, isobutyl, sec-butyl, tert-butyl, pentyl (branched or unbranched), hexyl (branched or unbranched), heptyl (branched or unbranched), octyl (branched or unbranched), nonyl (branched or unbranched), and decyl (branched or unbranched).

The term “alkenyl group” (alone or in combination with another term(s)) means a straight- or branched-chain hydrocarbon substituent containing one or more double bonds and typically 2 to 15 carbon atoms; such as 2 to 10, 2 to 8, 2 to 6 or 2 to 4 carbon atoms. Examples of such substituents include ethenyl (vinyl), 1-propenyl, 3-propenyl, 1,4-pentadienyl, 1,4-butadienyl, 1-butenyl, 2-butenyl, 3-butenyl, pentenyl and hexenyl.

The term “alkynyl group” (alone or in combination with another term(s)) means a straight- or branched-chain hydrocarbon substituent containing one or more triple bonds and typically 2 to 15 carbon atoms; such as 2 to 10, 2 to 8, 2 to 6 or 2 to 4 carbon atoms. Examples of such substituents include ethynyl, 1-propynyl, 3-propynyl, 1-butynyl, 3-butynyl and 4-butynyl.

The term “heteroalkyl group” (alone or in combination with another term(s)) means a straight- or branched-chain saturated hydrocarbyl substituent typically containing 1 to 15 atoms, such as 1 to 10, 1 to 8, 1 to 6, or 1 to 4 atoms, wherein at least one of the atoms is a heteroatom (i.e. oxygen, nitrogen, or sulfur), with the remaining atoms being carbon atoms. A “COₙ heteroalkyl” group refers to an aliphatic group containing n carbon atoms and one or more heteroatoms, for example one heteroatom. For example, a C₁-C₁₀ heteroalkyl group contains 1, 2, 3, 4, 5, 6, 7, 8, 9 or 10 carbon atoms in addition to one or more heteroatoms, for example one heteroatom. Attachment to the heteroalkyl group occurs through a carbon atom or through a heteroatom.

The term “heteroalkenyl group” (alone or in combination with another term(s)) means a straight- or branched-chain hydrocarbon substituent containing one or more carbon-carbon double bonds and typically 2 to 15 atoms; such as 2 to 10, 2 to 8, 2 to 6 or 2 to 4 atoms, wherein at least one of the atoms is a heteroatom (i.e. oxygen, nitrogen, or sulfur), with the remaining atoms being carbon atoms. A “Cₙ heteroalkenyl” group refers to an aliphatic group containing n carbon atoms and one or more heteroatoms, for example one heteroatom. For example, a C₂-C₁₀ heteroalkenyl group contains 2, 3, 4, 5, 6, 7, 8, 9 or 10 carbon atoms in addition to one or more heteroatoms, for example one heteroatom. Attachment to the heteroalkenyl group occurs through a carbon atom or through a heteroatom.

The term “heteroalkynyl group” (alone or in combination with another term(s)) means a straight- or branched-chain hydrocarbon substituent containing one or more carbon-carbon triple bonds and typically 2 to 15 carbon atoms; such as 2 to 10, 2 to 8, 2 to 6 or 2 to 4 carbon atoms, wherein at least one of the atoms is a heteroatom (i.e. oxygen, nitrogen, or sulfur), with the remaining atoms being carbon atoms. A “COₙ heteroalkynyl” group refers to an aliphatic group containing n carbon atoms and one or more heteroatoms, for example one heteroatom. For example, a C₂-C₁₀ heteroalkynyl group contains 1, 2, 3, 4, 5, 6, 7, 8, 9 or 10 carbon atoms in addition to one or more heteroatoms, for example one heteroatom. Attachment to the heteroalkynyl group occurs through a carbon atom or through a heteroatom.

The term “carbocyclyl group” (alone or in combination with another term(s)) means a saturated cyclic (i.e. “cycloalkyl”), partially saturated cyclic (i.e. “cycloalkenyl”), or completely unsaturated (i.e. “aryl”) hydrocarbon substituent containing from 3 to 14 carbon ring atoms (“ring atoms” are the atoms bound together to form the ring or rings of a cyclic substituent). A carbocyclyl may be a single-ring (monocyclic) or polycyclic ring structure.

A carbocyclyl may be a single ring structure, which typically contains 3 to 8 ring atoms, more typically 3 to 7 ring atoms, and more typically 5 to 6 ring atoms. Examples of such single-ring carbocyclyls include cyclopropyl (cyclopropanyl), cyclobutyl (cyclobutanyl), cyclopentyl (cyclopentanyl), cyclopentenyl, cyclopentadienyl, cyclohexyl (cyclohexanyl), cyclohexenyl, cyclohexadienyl, and phenyl. A carbocyclyl may alternatively be polycyclic (i.e. may contain more than one ring). Examples of polycyclic carbocyclyls include bridged, fused, and spirocyclic carbocyclyls. In a spirocyclic carbocyclyl, one atom is common to two different rings. An example of a spirocyclic carbocyclyl is spiropentanyl. In a bridged carbocyclyl, the rings share at least two common non-adjacent atoms. Examples of bridged carbocyclyls include bicyclo[2.2.1]heptanyl, bicyclo[2.2.1]hept-2-enyl, and adamantanyl. In a fused-ring carbocyclyl system, two or more rings may be fused together, such that two rings share one common bond. Examples of two- or three-fused ring carbocyclyls include naphthalenyl, tetrahydronaphthalenyl (tetralinyl), indenyl, indanyl (dihydroindenyl), anthracenyl, phenanthrenyl, and decalinyl.

The term “cycloalkyl group” (alone or in combination with another term(s)) means a saturated cyclic hydrocarbon substituent containing 3 to 14 carbon ring atoms. A cycloalkyl may be a single carbon ring, which typically contains 3 to 8 carbon ring atoms and more typically 3 to 6 ring atoms. It is understood that attachment to a cycloalkyl group is via a ring atom of the cycloalkyl group. Examples of single-ring cycloalkyls include cyclopropyl, cyclobutyl, cyclopentyl, and cyclohexyl. A cycloalkyl may alternatively be polycyclic or contain more than one ring. Polycyclic cycloalkyls include bridged, fused, and spirocyclic cycloalkyls.

The term “alkylcycloalkyl” refers to a cycloalkyl substituent attached via an alkyl chain. Examples of an alkylcycloalkyl substitent include cyclohexylethane, where the cyclohexane is attached via an ethane linker. Other examples include cyclopropylethane, cyclobutylethane, cyclopentylethane, cycloheptylethane, cyclohexylmethane. In a “Cₙ” alkylcycloalkyl, Cₙ includes the carbon atoms in the alkyl chain and in the cycloalkyl ring. For example, cyclohexylethane is a C8 alkylcycloalkyl.

The term “aryl group” (alone or in combination with another term(s)) means an aromatic carbocyclyl containing from 5 to 14 carbon ring atoms, optionally 5 to 8, 5 to 7, optionally 5 to 6 carbon ring atoms. A “Cₙ aryl” group refers to an aromatic group containing n carbon atoms. For example, a C₆-C₁₀ aryl group contains 6, 7, 8, 9 or 10 carbon atoms. Preferably an aryl group is a C6 aryl—i.e. phenyl. Attachment to the aryl group occurs through a carbon atom. An aryl group may be monocyclic or polycyclic (i.e. may contain more than one ring). In the case of polycyclic aromatic rings, only one ring in the polycyclic system is required to be unsaturated while the remaining ring(s) may be saturated, partially saturated or unsaturated. Attachment to the aryl group occurs through a carbon atom contained in the ring. Examples of aryl groups include phenyl, naphthyl, acridinyl, indenyl, indanyl, and tetrahydronapthyl.

The term “arylalkyl” refers to an aryl substituent attached via an alkyl chain. Examples of an arylalkyl substitent include benzyl and phenylethane/ethylbenzene, where the ethane chain links to a phenyl group to the point of attachment. In a “Cₙ” arylalkyl, Cₙ includes the carbon atoms in the alkyl chain and in the aryl group. For example, ethylbenzene is a C8 arylalkyl.

The term “heterocyclyl group” (alone or in combination with another term(s)) means a saturated (i.e. “heterocycloalkyl”), partially saturated (i.e. “heterocycloalkenyl”), or completely unsaturated (i.e. “heteroaryl”) ring structure containing a total of 3 to 14 ring atoms, wherein at least one of the ring atoms is a heteroatom (i.e. oxygen, nitrogen, or sulfur), with the remaining ring atoms being carbon atoms. A heterocyclyl group may, for example, contain one, two, three, four or five heteroatoms. Attachment to the heterocyclyl group may occur through a carbon atom and/or one or more heteroatoms that are contained in the ring. A heterocyclyl may be a single-ring (monocyclic) or polycyclic ring structure.

A heterocyclyl group may be a single ring, which typically contains from 3 to 7 ring atoms, more typically from 3 to 6 ring atoms, and even more typically 5 to 6 ring atoms. Examples of single-ring heterocyclyls include furanyl, dihydrofuranyl, tetrahydrofuranyl, thiophenyl (thiofuranyl), dihydrothiophenyl, tetrahydrothiophenyl, pyrrolyl, pyrrolinyl, pyrrolidinyl, imidazolyl, imidazolinyl, imidazolidinyl, pyrazolyl, pyrazolinyl, pyrazolidinyl, triazolyl, tetrazolyl, oxazolyl, oxazolidinyl, isoxazolidinyl, isoxazolyl, thiazolyl, isothiazolyl, thiazolinyl, isothiazolinyl, thiazolidinyl, isothiazolidinyl, thiodiazolyl, oxadiazolyl (including 1,2,3-oxadiazolyl, 1,2,4-oxadiazolyl, 1,2,5-oxadiazolyl (furazanyl) or 1,3,4-oxadiazolyl), oxatriazolyl, dioxazolyl oxathiolyl, pyranyl, dihydropyranyl, tetrahydropyranyl, thiopyranyl, tetrahydrothiopyranyl, pyridinyl (azinyl), piperidinyl, diazinyl (including pyridazinyl (1,2-diazinyl), pyrimidinyl (1,3-diazinyl) or pyrazinyl (1,4-diazinyl)), piperazinyl, triazinyl (including 1,3,5-triazinyl, 1,2,4-triazinyl and 1,2,3-triazinyl)), oxazinyl (including 1,2-oxazinyl, 1,3-oxazinyl or 1,4-oxazinyl)), oxadiazinyl (including 1,2,3-oxadiazinyl, 1,2,4-oxadiazinyl, 1,4,2-oxadiazinyl or 1,3,5-oxadiazinyl)), morpholinyl, azepinyl, oxepinyl, thiepinyl, and diazepinyl.

A heterocyclyl group may alternatively be polycyclic (i.e. may contain more than one ring). Examples of polycyclic heterocyclyl groups include bridged, fused, and spirocyclic heterocyclyl groups. In a spirocyclic heterocyclyl group, one atom is common to two different rings. In a bridged heterocyclyl group, the rings share at least two common non-adjacent atoms. In a fused-ring heterocyclyl group, two or more rings may be fused together, such that two rings share one common bond. Examples of fused ring heterocyclyl groups containing two or three rings include indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl. Other examples of fused-ring heterocyclyl groups include benzo-fused heterocyclyl groups, such as indolyl, isoindolyl (isobenzazolyl, pseudoisoindolyl), indoleninyl (pseudoindolyl), isoindazolyl (benzpyrazolyl), benzazinyl (including quinolinyl (1-benzazinyl) or isoquinolinyl (2-benzazinyl)), phthalazinyl, quinoxalinyl, quinazolinyl, benzodiazinyl (including cinnolinyl (1,2-benzodiazinyl) or quinazolinyl (1,3-benzodiazinyl)), benzopyranyl (including chromanyl or isochromanyl), benzofuranyl, dihydrobenzofuranyl, and benzisoxazinyl (including 1,2-benzisoxazinyl or 1,4-benzisoxazinyl).

The term “heterocycloalkyl group” (alone or in combination with another term(s)) means a saturated heterocyclyl. A “COₙ heterocycloalkyl” group refers to a cyclic aliphatic group containing n carbon atoms in addition to at least one heteroatom, for example nitrogen. For example, a C₁-C₁₀ heterocycloalkyl group contains 1, 2, 3, 4, 5, 6, 7, 8, 9 or 10 carbon ring atoms in addition to the at least one heteroatom. Attachment to the heterocycloalkyl group occurs through a carbon atom or one of the at least one heteroatoms.

The term “alkylheterocycloalkyl” refers to a heterocycloalkyl substituent attached via an alkyl chain. In a “Cₙ” alkylheterocycloalkyl, Cₙ includes the carbon atoms in the alkyl chain and in the heterocycloalkyl ring. For example, ethylpiperidine is a C7 alkylheterocycloalkyl.

The term “heteroaryl group” (alone or in combination with another term(s)) means an aromatic heterocyclyl containing from 5 to 14 ring atoms. A “Cₙ heteroaryl” group refers to an aromatic group containing n carbon atoms and at least one heteroatom. For example, a C₂-C₁₀ aryl group contains 2, 3, 4, 5, 6, 7, 8, 9 or 10 carbon atoms in addition to at least one heteroatom. Attachment to the heteroaryl group occurs through a carbon atom or through a heteroatom. A heteroaryl group may be monocyclic or polycyclic. A heteroaryl may be a single ring or 2 or 3 fused rings. Examples of monocyclic heteroaryl groups include 6-membered rings such as pyridyl, pyrazinyl, pyrimidinyl, pyridazinyl, and 1,3,5-, 1,2,4- or 1,2,3-triazinyl; 5-membered rings such as imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl. Polycyclic heteroaryl groups may be 2 or 3 fused rings. Examples of polycyclic heteroaryl groups include 6/5-membered fused ring groups such as benzothiofuranyl, benzisoxazolyl, benzoxazolyl, and purinyl; and 6/6-membered fused ring groups such as benzopyranyl, quinolinyl, isoquinolinyl, cinnolinyl, quinazolinyl, and benzoxazinyl. In the case of polycyclic heteroaryl groups, only one ring in the polycyclic system is required to be unsaturated while the remaining ring(s) may be saturated, partially saturated or unsaturated.

A nitrogen-containing heteroaryl group is a heteroaryl group in which at least one of the one or more heteroatoms in the ring is nitrogen.

The term “heteroarylalkyl” refers to a heteroaryl substituent attached via an alkyl chain. Examples of a heteroarylalkyl substitent include ethylpyridine, where the ethane chain links a pyridine group to the point of attachment.

The term “amino group” refers to the —NR^{m}Rⁿ group. The amino group can be optionally substituted. In an unsubstituted amino group, R^{m} and Rⁿ are hydrogen. In a substituted amino group R^{m} and Rⁿ each independently may be, but are not limited to, hydrogen, an alkyl, heteroalkyl, cycloalkyl, heterocycloalkyl, alkylcycloalkyl, alkylheterocycloalkyl, alkoxy, sulfonyl, alkenyl, alkanoyl, aryl, arylalkyl, or a heteroaryl group, provided R^{m} and Rⁿ are not both hydrogen. In a substituted amino group R^{m} and Rⁿ may cyclise to form a cyclic amino group, e.g. a pyrrolidine group or a piperidine group. Such a cyclic amino group may incorporate other heteroatoms, for example to form a piperazine or morpholine group. Such a cyclic amino group may be optionally substituted, e.g. with an amino group, a hydroxyl group or an oxo group. For example, in a substituted amino group R^{m} and Rⁿ may be independently selected from H; C1-C3 alkyl optionally substituted with OH or halo; C3-C4 cycloalkyl optionally substituted with methyl and/or halo; C3-C4 heterocycloalkyl optionally substituted with oxo, methyl or fluoro-methyl; C3-C5 heteroaryl optionally substituted with methyl; Boc; COOH; and COOCH3; provided at least one of R^{m} and Rⁿ is not H.

The term “aminoalkyl” group refers to the —R^{a}NRₘRₙ group, wherein R^{a} is an alkyl chain as defined above and NRₘRₙ is an optionally substituted amino group as defined above. “Cₙ aminoalkyl” group refers to a group containing n carbon atoms. For example, a C₁-C₁₀ aminoalkyl group contains 1, 2, 3, 4, 5, 6, 7, 8, 9 or 10 carbon atoms. When the amino group of the aminoalkyl group is a substituted amino group, the number of carbon atoms includes any carbon atoms in the substituent groups. Attachment to the aminoalkyl group occurs through a carbon atom of the R alkyl group. Examples of aminoalkyl substituents include methylamine, ethylamine, methylaminomethyl, dimethylaminomethyl, methylaminoethyl, dimethylaminoethyl, methylpyrrolidine, and ethylpyrrolidine

The term “amido group” refers to the —C(═O)—NR— group. Attachment may be through the carbon or nitrogen atom. For example, the amido group may be attached as a substituent via the carbon atom only, in which case the nitrogen atom has two R groups attached (—C(═O)—NR₂). The amido group may be attached by the nitrogen atom only, in which case the carbon atom has an R group attached (—NR—C(═O)R).

The term sulfoximine refers to sulfoximine substituents that are either S-linked or N-linked—that is, attachment may be through the sulfur or nitrogen atom. For example, the sulfoximine group may be attached as a substituent via the sulfur atom, in which case the sulfur has a single R group in addition to the oxo group and the sulfur-bound nitrogen atom has one R group attached—that is the group is —S(O)(R)NR′. By way of further example, the sulfoximine group may be attached as a substituent via the nitrogen atom, in which case the sulfur atom has two attached R groups in addition to the oxo group—that is, the group is —NS(O)RR′. In unsubstituted sulfoximine groups, each of R and R′ are H. Alternatively, the sulfoximine group may be substituted at one or both of R and R′, for example to form a dimethyl sulfoximine, where both R and R′ are methyl.

The term “ether” refers to an —O-alkyl group or an -alkyl-O-alkyl group, for example a methoxy group, a methoxymethyl group or an ethoxyethyl group. The alkyl chain(s) of an ether can be linear, branched or cyclic chains. The ether group can be optionally substituted (a “substituted ether”) with one or more substituents. A C ether refers to an ether group having n carbons in all alkyl chains of the ether group. For example, a CH(CH3)-O—C6H11 ether is a C₈ ether group.

The term “alkoxy group” refers to an —O-alkyl group. The alkoxy group can refer to linear, branched, or cyclic, saturated or unsaturated oxy-hydrocarbon chains, including, for example, methoxyl, ethoxyl, propoxyl, isopropoxyl, butoxyl, t-butoxyl and pentoxyl. The alkoxy group can be optionally substituted (a “substituted alkoxy”) with one or more alkoxy group substituents.

The term “aryloxy group” refers to an —O-aryl group, for example a phenoxy group. An aryloxy substituent may itself be optionally substituted, for example with a halogen.

The term “alkylester” refers to a —C(O)OR group, where R is an alkyl group as defined herein. An example of an alkylester is ethyl methanoate—i.e. R is an ethyl group.

The term “hydroxyl” refers to an —OH group.

The term “oxo group” refers to the (═O) group, i.e. a substituent oxygen atom connected to another atom by a double bond. For example, a carbonyl group (—C(═O)—) is a carbon atom connected by a double bond to an oxygen atom, i.e. an oxo group attached to a carbon atom. Examples of carbonyl substituents include aldehydes (—C(═O)H), acetyl (—C(═O)CH3) and carboxyl/carboxylic acid groups (—C(═O)OH).

The term “halo” refers to a substituent selected from chlorine, fluorine, bromine and iodine. Preferably, the halo substituent is selected from chlorine and fluorine.

An alkyl, alkenyl, alkynyl, carbocyclyl (including cycloalkyl, cycloalkenyl and aryl), heterocyclyl (including heterocycloalkyl, heterocyloalkenyl, heteroaryl, nitrogen-containing heterocyclyl), amino, amido, ester, ether, alkoxy, or sulfonamide group can be optionally substituted with one or more substituents, which can be the same or different. A substituent can be attached through a carbon atom and/or a heteroatom in the alkyl, alkenyl, alkynyl, carbocyclyl (including cycloalkyl, cycloalkenyl and aryl), heterocyclyl (including heterocycloalkyl, heterocyloalkenyl, heteroaryl, nitrogen-containing heterocyclyl, nitrogen-containing heteroaryl), amino, amido, ester, ether, alkoxy, or sulfonamide group. The term “substituent” (or “radical”) includes but is not limited to alkyl, substituted alkyl, heteroalkyl, substituted heteroalkyl, aralkyl, substituted aralkyl, alkenyl, substituted alkenyl, alkynyl, substituted alkynyl, halo, hydroxyl, cyano, amino, amido, alkylamino, arylamino, carbocyclyl, cycloalkyl, substituted cycloalkyl, heterocycloalkyl, substituted heterocycloalkyl, cycloalkenyl, substituted cycloalkenyl, aryl, substituted aryl, heteroaryl, substituted heteroaryl, nitro, thio, alkanoyl, hydroxyl, aryloxyl, alkoxyl, alkylthio, arylthio, aralkyloxyl, aralkylthio, carboxyl, alkoxycarbonyl, oxo, alkylsulfonyl, arylsulfonyl and sulfoximinyl.

In certain aspects, the substituent is alkyl, substituted alkyl, heteroalkyl, substituted heteroalkyl, alkenyl, substituted alkenyl, alkynyl, substituted alkynyl, halo, hydroxyl, cyano, amino, amido, alkylamino, arylamino, carbocyclyl, cycloalkyl, substituted cycloalkyl, heterocycloalkyl, substituted heterocycloalkyl, cycloalkenyl, substituted cycloalkenyl, aryl, substituted aryl, heteroaryl, substituted heteroaryl, nitro, thio, alkanoyl, hydroxyl, aryloxyl, alkoxyl, alkylthio, arylthio, aralkyloxyl, aralkylthio, carboxyl, alkoxycarbonyl, oxo, alkylsulfonyl and arylsulfonyl.

If a group, for example an alkyl group, is “optionally substituted”, it is understood that the group has one or more substituents attached (substituted) or does not have any substituents attached (unsubstituted).

If a group is substituted with a further optionally substituted group, it is understood that the first substituent may itself be either unsubstituted or substituted.

For completeness, it is also noted that certain chemical formulae used herein define delocalized systems. This definition is known in the art as a definition of aromaticity and may indicate the presence of, for example, a planar mono-, di- or tri-cyclic system that contains (4n+2) electrons where n is an integer. In other words, these systems may display Hückel aromaticity.

In whatever aspect, the compounds of the present invention may possess some aspect of stereochemistry. For example, the compounds may possess chiral centres and/or planes and/or axes of symmetry. As such, the compounds may be provided as single stereoisomers, single diastereomers, mixtures of stereoisomers or as racemic mixtures, unless otherwise specified. Stereoisomers are known in the art to be molecules that have the same molecular formula and sequence of bonded atoms, but which differ in their spatial orientations of their atoms and/or groups.

In addition, the compounds of the present invention may exhibit tautomerism. Each tautomeric form is intended to fall within the scope of the invention.

In addition, the compounds of the present invention may be provided as a pro-drug. Pro-drugs are transformed, generally in vivo, from one form to the active forms of the drugs described herein.

In addition, it will be understood that the elements described herein may be the common isotope or an isotope other than the common isotope. For example, a hydrogen atom may be ¹H, ²H (deuterium) or ³H (tritium).

In addition, the compounds of the present invention may be provided in the form of their pharmaceutically acceptable salts or as co-crystals.

The term “pharmaceutically acceptable salt” refers to ionic compounds formed by the addition of an acid to a base. The term refers to such salts that are considered in the art as being suitable for use in contact with a patient, for example in vivo and pharmaceutically acceptable salts are generally chosen for their non-toxic, non-irritant characteristics.

The term “co-crystal” refers to a multi-component molecular crystal, which may comprise non-ionic interactions.

Pharmaceutically acceptable salts and co-crystals may be prepared by ion exchange chromatography or by reacting the free base or acidic form of a compound with stoichiometric amounts or with an excess of the desired salt-forming inorganic or organic acid or base in one or more suitable solvents, or by mixing the compound with another pharmaceutically acceptable compound capable of forming a co-crystal.

Salts known in the art to be generally suitable for use in contact with a patient include salts derived from inorganic and/or organic acids, including the hydrobromide, hydrochloride, sulfate, bisulfate, nitrate, acetate, oxalate, oleate, palmitate, stearate, laurate, benzoate, lactate, phosphate, tosylate, citrate, maleate, fumarate, succinate and tartrate. These may include cations based on the alkali and alkaline earth metals, such as sodium, potassium, calcium and magnesium, as well as ammonium, tetramethylammonium, tetraethylammonium. Further reference is made to the number of literature sources that survey suitable pharmaceutically acceptable salts, for example the handbook of pharmaceutical salts published by IUPAC.

In addition, the compounds of the present invention may sometimes exist as zwitterions, which are considered as part of the invention.

As used herein, a USP19 inhibitor refers to a compound which acts on USP19 so as to decrease the activity of the enzyme. Examples of USP19 inhibitors are exemplified compounds herein. Preferably a USP19 inhibitor exhibits an IC₅₀ of less than 5 μM, preferably less than 0.5 μM.

As used herein, “obesity” refers to the medical condition characterised by excess body fat. Obesity can be characterised by, for example, a body mass index (BMI) of greater than 30. Treatment of obesity may be indicated by, for example, the reduction of body fat, in percentage and/or absolute mass terms. Treatment of obesity may also be exemplified by a reduction in the rate of body fat accumulation by a subject compared to before treatment.

As used herein, “insulin resistance” refers to the medical condition characterised by an abnormally weak response to insulin. Since insulin resistance is typically not treated by exogenous insulin treatment, the resistance is typically to insulin produced by the body of the subject, though the subject may also be resistant to exogenous insulin. “Insulin resistance” encompasses the conditions “prediabetes” and Type II diabetes. Insulin resistance may be indicated, for example, by a glucose tolerance test (GTT) glycaemia of 7.8 mmol/L or greater. Type II diabetes is typically diagnosed following a glucose tolerance test (GTT) glycaemia of 11.1 mmol/L or greater.

Treatment of insulin resistance may be indicated by an improvement (i.e. reduction) in the subject's GTT glycaemia compared to before treatment. Treatment may also be indicated by a reduction in the subject's blood sugar concentration under normal conditions compared to before treatment.

As used herein, “muscular atrophy” and “muscle-wasting” are used interchangeably to refer to decrease in muscle mass in a subject, including in the context of cachexia or sarcopenia, for example. Muscular atrophy can be as a result of temporary or permanent disability, temporary or permanent immobilisation of a limb, extended bedrest, cachexia (for example as a result of cancer, heart failure, or COPD), or sarcopenia.

Treatment of muscular atrophy may be characterised as the slowing of the rate of atrophy—that is, treatment results in less muscle mass lost over a given period of time. Preferably, successful treatment results in no loss of muscle mass.

Accordingly, in a first aspect is provided a compound of formula (I):

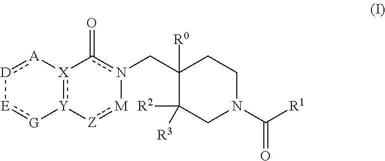

wherein
R⁰ is H, F, NH2, or OCH3;
R¹ is optionally substituted C1-C6 alkyl, ethylcyclopropyl, ethylcyclobutyl, optionally substituted C5-C8 aryl, optionally substituted C3-C8 heteroaryl, optionally substituted C3-C8 heterocycloalkyl, NR^{a}R^{b}, NR^{a}CH2R^{b}, OR^{a}, or OCH2R^{a}, wherein R^{a} and R^{b} are independently selected from H, C1-C6 alkyl, CF3, optionally substituted C3-C6 cycloalkyl, optionally substituted C5-C8 aryl, optionally substituted C6-C9 arylalkyl, and optionally substituted C2-C8 heteroaryl (optionally C4-C8 heteroaryl), and wherein when R¹ is NR^{a}CH2R^{b}, the methylene group may be optionally substituted with CF3, or wherein R¹ is NR^{a}R^{b} and R^{a} and R^{b} together form an optionally substituted C2-C9 heterocycle together with the N to which they are attached, optionally together form a C3-C5 heterocycle together with the N to which they are attached;
R² and R³ are independently selected from H, and C1-C6 alkyl, or together form a C3-C6 cycloalkyl or heterocycloalkyl with the carbon to which they attached;
X is absent, C, CR^{4a}, CR^{4a}R^{4b}, N, NR^{4a}, or C═O,
   wherein R^{4a} and R^{4b} are independently selected from H, optionally substituted C1-C6 alkyl or halo;
   or wherein R^{4a} and R^{4b} together form a C3-C6 cycloalkyl or C3-C6 heterocycloalkyl including the carbon to which they are attached;
Y is C, CR⁵, CR⁵R⁶, N, NR⁵, or O,
   wherein R⁵ and R⁶ are independently selected from H, halo, optionally substituted C1-C6 alkyl, C2-C6 alkenyl, C2-C6 alkynyl, optionally substituted C3-C6 cycloalkyl, optionally substituted C3-C6 heterocycloalkyl, optionally substituted C5-C8 aryl, optionally substituted C6-C9 arylalkyl, optionally substituted C3-C8 heteroaryl, CH2OH, NR′R″, NS(O)R′R″, SO2R′, C(O)R′, COR′, C(O)OR′, C(O)NR′R′, OR′, wherein R′ and R″ are independently selected from H, C1-C6 alkyl, C5-C8 aryl, C6-C9 arylalkyl, and C3-C8 heteroaryl, or wherein R⁵ is NR′R″ and R′ and R″ together form an optionally substituted C3-6 heterocycloalkyl including the nitrogen to which they are attached
   or wherein R⁵ and R⁶ together form a C3-6 cycloalkyl or C3-C6 heterocycloalkyl including the carbon to which they are attached;
Z is N, NR⁷, C, CR⁷, CR⁷R⁸, or C═O,
   wherein R⁷ and R⁸ are independently selected from H, halo, cyano, oxo, optionally substituted C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, C(O)OR^{c}, CONR^{c}R^{d}, NR^{c}R^{d}, NS(O)R^{c}R^{d}, S(O)(R^{c})NR^{d}, SOR^{c}, SO2R^{c}, and SR^{c}, wherein R^{c} and R^{d} are independently H, C1-C6 alkyl, C3-C6 cycloalkyl, C5-C6 aryl, C6-C9 arylalkyl, C3-C6 heteroaryl, CN, COOH, or COCH3 or R^{c} and R^{d} together form an optionally substituted C3-C7 heterocycle together with the heteroatom to which they are attached;
   or wherein R⁷ and R⁸ together form a C3-6 cycloalkyl or C3-C6 heterocycloalkyl including the carbon to which they are attached;
M is absent, C, CR¹³ or CR¹³R¹⁴, wherein R¹³ and R¹⁴ are independently selected from H, and C1-C6 alkyl, or wherein R¹³ and R¹⁴ together form a C3-C6 cycloalkyl or C3-C6 heterocycloalkyl together with the carbon to which they are attached; and
A is CR⁹, CHR⁹, N, NR⁹, S, or O,
D is CR⁹, CHR⁹, N or NR⁹,
G is absent, CR⁹, CHR⁹, or N,
   wherein R⁹ is independently selected from H, halo, C1-C6 alkyl, CF3, and OR*, wherein R* is an optionally substituted C1-C6 alkyl, optionally substituted C3-C6 cycloalkyl or optionally substituted heterocycloalkyl,
E is CR¹⁰, CHR¹⁰, N, NR¹⁰, S, or O,
   wherein R¹⁰ is selected from H, halo, C1-C6 alkyl, C3-C6 cycloalkyl, C5-C8 aryl, C6-C9 arylalkyl, C4-C8 heteroaryl, SR^{x}, OR^{x}, NR^{x}R^{y}, and NS(O)R^{x}R^{y}, S(O)(R^{x})NR^{y}, wherein R^{x} and R^{y} are independently selected from H, C1-C6 alkyl, CF3, C3-C6 cycloalkyl, C5-C8 aryl, C6-C9 arylalkyl, C4-C8 heteroaryl, COOH, amido, cyano, C2-C6 alkene, C2-C6 alkyne, or wherein R^{x} and R^{y} together form an optionally substituted C4-C6 heterocycloalkyl together with the nitrogen to which they are attached;
or A, D, E and G are all absent, and X, Y, Z and M are as defined above, and optionally wherein both X and M are absent, or
optionally wherein Y and Z together form an optionally substituted C5-C6 aryl or C3-C6 heteroaryl fused ring or Z and M together form an optionally substituted C5-C6 aryl or C5-C6 heteroaryl fused ring;
or a compound having a structure selected from:

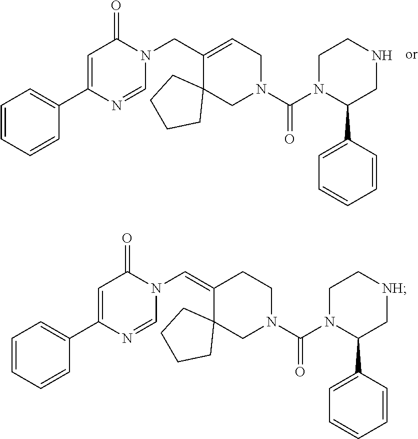 [SMILES: O=C(N1CC=C(Cn2cnc(-c3ccccc3)cc2=O)C2(CCCC2)C1)N1CCNC[C@H]1c1ccccc1 ; "or" ; O=C(N1CC/C(=C/n2cnc(-c3ccccc3)cc2=O)C2(CCCC2)C1)N1CCNC[C@H]1c1ccccc1]

or a stereoisomer, tautomer, hydrate, N-oxide derivative or pharmaceutically acceptable salt thereof.

For the avoidance of doubt, in formula (I), if any of positions A, D, E or G are present, each of the other positions A, D and E (and optionally G) are also present to form a fused ring system.

For the avoidance of doubt, when one of positions X, M or G are absent, the remaining members of the ring form a 5 membered ring. In those embodiments in which X and M are absent, the remaining members form a 4 membered ring. For instance, if M is absent, the atom at ring position Z is bound to the ring nitrogen.

Dotted lines in formula (I) indicate optional bonds. That is, the dotted lines indicate the ring including positions X, Y, Z, M can be aliphatic (for example saturated or partially unsaturated) or aromatic. Similarly, in formula (I) dotted lines indicate that, when present, the ring including positions A, D, E and optionally G can be aliphatic (for example saturated or partially unsaturated) or aromatic.

For the compounds of formula (I), for each group that is optionally substituted, there is one or more independently selected optional substituent. In certain embodiments, each of the one or more optional substituents is independently selected from C1-C4 alkyl, C3-C4 cycloalkyl, halo, CHF2, CF3, hydroxyl, NH2, substituted amino, NO2, CH2OH, CH2OCH3, methoxy, OCHF2, OCF3, cyclopropyloxy, phenyl, fluoro-substituted phenyl (e.g difluoro-substituted phenyl), benzyl, and oxo.

In certain embodiments each of the one or more optional substituents is independently selected from C1-C4 alkyl, C3-C4 cycloalkyl, halo, CHF2, CF3, hydroxyl, NH2, NHCH3, NHCH2CH3, NO2, CH2OH, CH2OCH3, methoxy, OCHF2, OCF3, cyclopropyloxy, phenyl, fluoro-substituted phenyl (e.g difluoro-substituted phenyl), benzyl, and oxo.

In embodiments where R⁰ is H and R² and/or R³ are not H, the ring carbon to which R0 is attached is chiral. In accordance with the nomenclature convention described in the Examples, the (R)-configuration at the ring carbon is assigned to the more active stereoisomer for exemplified compounds where R0 is H and the ring carbon is chiral. When R0 is F, NH2 or OMe and the ring carbon to which R0 is attached is chiral, the more active conformation at that stereocentre is assigned the (S)-configuration in the exemplified compounds.

In embodiments of the invention where the compounds of formula (I) are chiral at the stereocentre at the ring carbon to which R0 is attached, the more active configuration is preferred.

In embodiments where R0 is H, preferably this stereocentre is in the (R)-configuration.

In embodiments where R0 is not H, preferably this stereocentre is in the (S)-configuration.

It should nevertheless be noted that it may be the case that any or all of the configurations at the R0 position have been incorrectly assigned, for example due to an error in the determination of the original X-ray crystallography data or in the strategy of inferring the stereochemistry from other compounds. Therefore, it is possible that these compounds have the opposite configuration at this position. As already described, the more active configuration is preferred. Accordingly, in embodiments where R0 is H, preferably the stereocentre at the ring carbon is in the (R)-configuration. In embodiments where R0 is not H, preferably this stereocentre is in the (S)-configuration.

TABLE 1
Comparison of target engagement potency (HTRF, HEK293T cells), caco-2 permeability
(A:B) and thermodynamic solubility (TSol) between R⁰ = OH and R⁰ = H matched pairs.

| | | HTRF TE | caco-2 Papp | TSol |
|---|---|---|---|---|
| Structure | Example | IC50 (μM) | AB (10⁻⁶ cm/s) | (μM) |
| 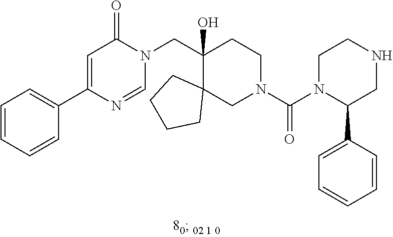 [SMILES: O=C(N1CC[C@@](O)(Cn2cnc(-c3ccccc3)cc2=O)C2(CCCC2)C1)N1CCNC[C@H]1c1ccccc1] | F 31 3 9N W 2 | | 23 | |
| 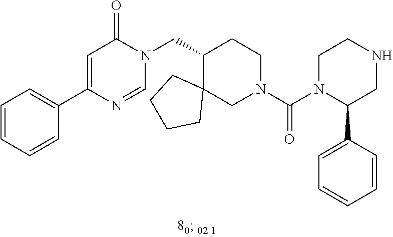 [SMILES: O=C(N1CC[C@@H](Cn2cnc(-c3ccccc3)cc2=O)C2(CCCC2)C1)N1CCNC[C@H]1c1ccccc1] | | 1 | 1 | |
| 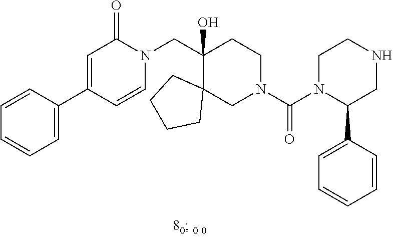 [SMILES: O=C(N1CC[C@@](O)(Cn2ccc(-c3ccccc3)cc2=O)C2(CCCC2)C1)N1CCNC[C@H]1c1ccccc1] | F 31 3 9N W 2 | 23 | 1 | 1 |
| 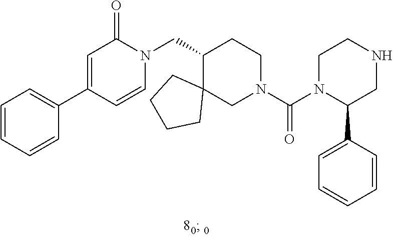 [SMILES: O=C(N1CC[C@@H](Cn2ccc(-c3ccccc3)cc2=O)C2(CCCC2)C1)N1CCNC[C@H]1c1ccccc1] | | 03 | 1 | 1 |
| 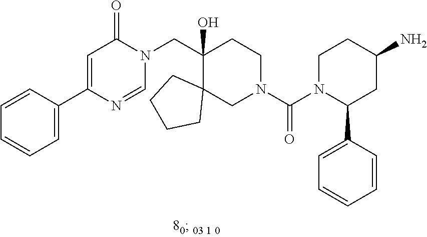 [SMILES: N[C@@H]1CCN(C(=O)N2CC[C@@](O)(Cn3cnc(-c4ccccc4)cc3=O)C3(CCCC3)C2)[C@H](c2ccccc2)C1] | F 11 9 N W032 | | ON W | 01 |
| 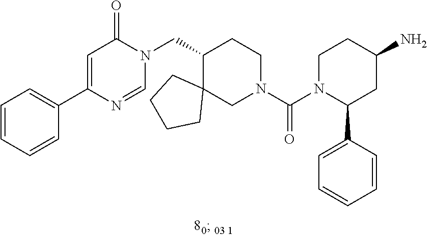 [SMILES: N[C@@H]1CCN(C(=O)N2CC[C@@H](Cn3cnc(-c4ccccc4)cc3=O)C3(CCCC3)C2)[C@H](c2ccccc2)C1] | 1 | 1 | 23 | 1 |

In certain embodiments of the compound of formula (I), R⁰ is H. As shown in Table 1, compounds wherein R⁰ is H exhibit improved cellular target engagement potency (HTRF assay in HEK293T cells) and improved in vitro ADME properties, such as caco-2 permeability (AB: apical to basolateral data shown) and thermodynamic solubility (TSol) compared to their direct analogues in which R⁰ is OH (some of which were previously reported in WO2019150119 or WO2020115501). In addition, compounds wherein R⁰ is H exhibited improved in vivo mouse PK data with respect to increased IV half life (t½ @ IV 1 mg/kg), reduced IV clearance (CL @ IV 1 mg/kg), and greatly superior oral exposure (area under the curve, AUC @ PO 30 mg/kg) and oral bioavailability (F @ PO 30 mg/kg) compared to its direct analogue in which R⁰ is OH, outlined in Table 2.

TABLE 2
Comparison of in vivo mouse PK data (C57BL/6N mice; vehicle: 2:98 DMSO/20% 2-
hydroxypropyl-β-cyclodextrin; IV 1 mg/kg; PO 30 mg/kg) between R⁰ = OH and R⁰ = H matched
pairs.

| | | IV | CL | AUC | F |
|---|---|---|---|---|---|
| Structure | Example | t1/2 (h) | (mL/min/kg) | (h*ng/mL) | (%) |
| 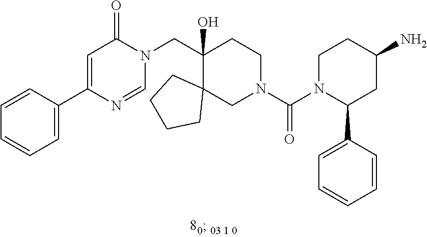 [SMILES: N[C@@H]1CCN(C(=O)N2CC[C@@](O)(Cn3cnc(-c4ccccc4)cc3=O)C3(CCCC3)C2)[C@H](c2ccccc2)C1] | F 11 9 N W032 | 2 | 3 | 3 | |
| 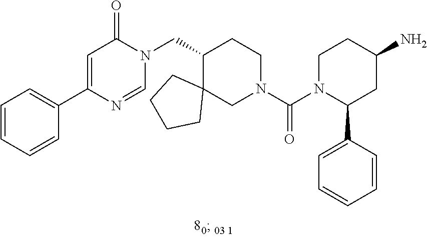 [SMILES: N[C@@H]1CCN(C(=O)N2CC[C@@H](Cn3cnc(-c4ccccc4)cc3=O)C3(CCCC3)C2)[C@H](c2ccccc2)C1] | 1 | | 1 | 113 | 1 |

In certain preferred embodiments of the compounds of formula (I), R⁰ is H.

In certain embodiments of compounds of formula (I), R⁰ is NH2. Compounds having NH2 at position R⁰ exhibit improved kinetic solubility (KSol) and/or metabolic stability (demonstrated by lower predicted hepatic clearance, CLhep, using mouse liver microsome data) compared to analogues having OH at position R⁰. This is shown in Table 3.

TABLE 3
Comparison of kinetic solubility (KSol) and predicted hepatic clearance (using mouse
liver microsome data) between R⁰ = OH and R⁰ = NH2 matched pair.

| | | KSol | HLM CLhep |
|---|---|---|---|
| Structure | Example | (μM) | (mL/min/kg) |
| 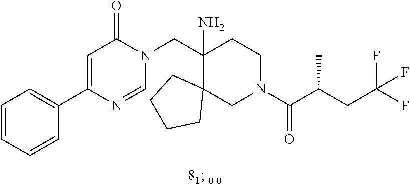 [SMILES: C[C@H](CC(F)(F)F)C(=O)N1CCC(N)(Cn2cnc(-c3ccccc3)cc2=O)C2(CCCC2)C1] | 3 | 3 | 3 |
| 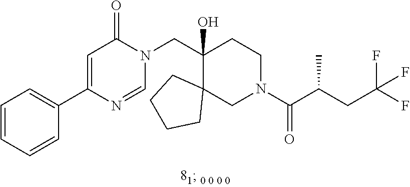 [SMILES: C[C@H](CC(F)(F)F)C(=O)N1CC[C@@](O)(Cn2cnc(-c3ccccc3)cc2=O)C2(CCCC2)C1] | F 11 9 N W 11 | 1 | 0 |

Accordingly, in certain preferred such embodiments, R⁰ is NH2.

In certain preferred embodiments, R⁰ is F.

In certain preferred embodiments, R⁰ is OCH3.

In certain preferred embodiments, R¹ is optionally substituted C1-C6 alkyl. In certain embodiments, the optional substituents are selected from halo, C1-C6 alkyl, C1-C6 alkoxy, and OH.

In certain preferred embodiments of the compound of formula (I), R¹ is optionally substituted trifluoropropyl. In certain preferred such embodiments, each optional substituent is selected from methyl, CH2OCH3 and CH2OH.

In certain preferred embodiments, R¹ is:

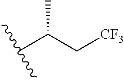 [SMILES: *[C@H](C)CC(F)(F)F]

In certain preferred embodiments, R¹ is:

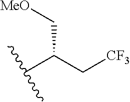 [SMILES: *[C@H](COC)CC(F)(F)F]

In certain preferred embodiments wherein R¹ is optionally substituted trifluoropropyl, R⁰ is H.

In certain preferred embodiments wherein R¹ is optionally substituted trifluoropropyl, R⁰ is NH2.

In certain preferred embodiments of the compound of formula (I) R¹ is NR^{a}R^{b} or NR^{a}CH2R^{b}, wherein R^{a} and R^{b} are independently selected from H, methyl, ethyl, propyl, CF3, optionally substituted cyclopropyl, optionally substituted cyclobutyl, optionally substituted cycopentyl, optionally substituted cyclohexyl, optionally substituted phenyl, optionally substituted benzyl, optionally substituted pyridinyl, pyrazole, imidazole, furan, benzodioxol, optionally substituted oxadiazole, thiazole, and thiophene, wherein each of the one or more optional substituents are independently selected from halo, methyl, cyclopropyl and CN,
optionally wherein R¹ is NR^{a}CH2R^{b} and the methylene group is substituted with CF3. or

In certain embodiments R¹ is NR^{a}R^{b} and R^{a} and R^{b} together form an optionally substituted C3-C9 heterocycle together with the N to which they are attached.

In certain embodiments, R¹ is NR^{a}R^{b} and R^{a} and R^{b} together form an optionally substituted C3-C9 heterocycle together with the N to which they are attached, wherein each of the one or more optional substituents is selected from OH, oxo, C1-C3 alkyl optionally substituted with OH and/or halo, optionally substituted phenyl, optionally substituted benzyl, C1-C3 alkoxy, NR^{m}Rⁿ, NHC(O)R^{m}, and NHCH2R¹,
wherein R^{m} and Rⁿ are independently selected from H; C1-C3 alkyl optionally substituted with OH, methoxy or halo; C3-C4 cycloalkyl optionally substituted with methyl and/or halo; C3-C4 heterocycloalkyl optionally substituted with oxo, methyl or fluoro-methyl; C3-C5 heteroaryl optionally substituted with methyl; and Boc; and/or wherein Rⁿ is further selected from CH2OCH3, COOH and COOCH3,
or wherein R^{m} and Rⁿ form a C3-C5 heterocyclyl group together with the N to which they are attached, optionally wherein R^{m} and Rⁿ form a morpholinyl group together with the N to which they are attached.

In certain embodiments, R¹ is NR^{a}R^{b} and R^{a} and R^{b} together form an optionally substituted C3-C9 heterocycle together with the N to which they are attached, wherein each of the one or more optional substituents is selected from optionally halo-substituted phenyl, NR^{m}Rⁿ, NHC(O)R^{m}, and NHCH2Rⁿ,
Wherein R^{m} and Rⁿ are independently selected from H; C1-C3 alkyl optionally substituted with OH, methoxy or halo; C3-C4 cycloalkyl optionally substituted with methyl and/or halo; C3-C4 heterocycloalkyl optionally substituted with oxo, methyl or fluoro-methyl; C3-C5 heteroaryl optionally substituted with methyl; and Boc; and/or wherein Rⁿ is further selected from CH2OCH3, COOH and COOCH3,
or wherein R^{m} and Rⁿ form a C3-C5 heterocyclyl group together with the N to which they are attached, optionally wherein R^{m} and Rⁿ form a morpholinyl group together with the N to which they are attached.

In certain embodiments, R¹ is NR^{a}R^{b} and R^{a} and R^{b} together form a substituted C3-C9 heterocycle together with the N to which they are attached, wherein each of the one or more substituents is selected from OH, CH2OH, CH2OCH3, oxo, NH2, C1-C3 aminoalkyl, amino-thietane dioxide, methyl, ethyl, propyl, CF3, phenyl, substituted phenyl, and benzyl.

In certain embodiments, R¹ is NR^{a}R^{b} and R^{a} and R^{b} form an optionally substituted heterocycle together with the N to which they are attached, wherein the heterocycle is selected from pyrrolidinyl, pyrimidinyl, piperidinyl, morpholino, piperazinyl, and thiomorpholino. In certain such embodiments, the heterocycle is optionally substituted with one or more substituents independently selected from methyl, spiro-cyclopropyl, C1-C3 aminoalkyl, NH2, CH2OH, CH2CF3, oxo, thiophene, phenyl optionally substituted with F or CF3, and OH provided the same ring carbon is not also substituted with methyl.

In certain embodiments, R¹ is NR^{a}R^{b} and R^{a} and R^{b} form a heterocycle together with the N to which they are attached, wherein the heterocycle is selected from pyrrolidinyl, piperidinyl, morpholino, piperazinyl, and thiomorpholino,
wherein the heterocycle is optionally substituted with one or more substituents independently selected from methyl, NH2, C1 or C2 aminoalkyl, CH2CF3, oxo, thiophene, phenyl optionally substituted with F or CF3, and OH provided the same ring carbon is not also substituted with methyl.

In certain embodiments, R¹ is NR^{a}R^{b} and R^{a} and R^{b} form a heterocycle together with the N to which they are attached, wherein the heterocycle is selected from piperidinyl and piperazinyl,
wherein the heterocycle is optionally substituted with one or more substituents independently selected from methyl, NH2, C1 or C2 aminoalkyl, CH2CF3, oxo, thiophene, phenyl optionally substituted with F or CF3, and OH provided the same ring carbon is not also substituted with methyl.

In certain embodiments, R¹ is NR^{a}R^{b} and R^{a} and R^{b} form an optionally substituted heterocycle together with the N to which they are attached, wherein the heterocycle is selected from piperidinyl and piperazinyl.

Preferably R¹ forms a piperazinyl group substituted with fluoro-phenyl or difluorophenyl.

In certain embodiments, the piperazinyl group is optionally further substituted with methyl.

In certain embodiments, the piperazinyl group is optionally further substituted with CH2OH or spiro-cyclopropyl.

In certain embodiments, R¹ is NR^{a}R^{b} and R^{a} and R^{b} form an optionally substituted heterocycle, wherein the heterocycle is a piperidinyl group substituted with phenyl. In certain preferred such embodiments the piperidinyl group is optionally further substituted with NH2 or NHCH3.

In certain embodiments, R¹ is NR^{a}R^{b} and R^{a} and R^{b} together with the N to which they are attached form a piperidinyl group optionally substituted with phenyl, fluoro-phenyl, or difluoro-phenyl, and wherein the piperidinyl group is optionally further substituted with NR^{m}Rⁿ, NHC(O)R^{m}, or NHCH2R¹,
Wherein R^{m} and Rⁿ are independently selected from H; C1-C3 alkyl optionally substituted with OH, methoxy or halo; C3-C4 cycloalkyl optionally substituted with methyl and/or halo; C3-C4 heterocycloalkyl optionally substituted with oxo, methyl or fluoro-methyl; C3-C5 heteroaryl optionally substituted with methyl; and Boc; and/or wherein Rⁿ is further selected from CH2OCH3, COOH and COOCH3,
or wherein R^{m} and Rⁿ form a C3-C5 heterocyclyl group together with the N to which they are attached, optionally wherein R^{m} and Rⁿ form a morpholinyl group together with the N to which they are attached.

In certain embodiments R¹ is NR^{a}R^{b} and R^{a} and R^{b} together with the N to which they are attached form a piperidinyl group optionally substituted with phenyl, fluoro-phenyl, or difluoro-phenyl, and wherein the piperidinyl group is optionally further substituted with NR^{m}Rⁿ, NHC(O)R^{m}, or NHCH2Rⁿ,
wherein R^{m} is selected from H; C1-C3 alkyl optionally substituted with OH or halo; C3-C4 cycloalkyl optionally substituted with methyl and/or halo; C3-C4 heterocycloalkyl optionally substituted with oxo, methyl or fluoro-methyl; C3-C5 heteroaryl optionally substituted with methyl; and Boc; and
wherein Rⁿ is selected from H; C1-C3 alkyl optionally substituted with OH or halo; C3-C4 cycloalkyl optionally substituted with methyl and/or halo; C3-C4 heterocycloalkyl optionally substituted with oxo, methyl or fluoro-methyl; C3-C5 heteroaryl optionally substituted with methyl; Boc; COOH and COOCH3.

In certain preferred embodiments, the piperidinyl ring formed by R¹ is substituted with NR^{m}Rⁿ, wherein R^{m} and Rⁿ are independently selected from H; C1-C3 alkyl optionally substituted with OH or halo (preferably F); C3-C4 cycloalkyl optionally substituted with methyl and/or halo (preferably F); C3-C4 heterocycloalkyl optionally substituted with oxo, methyl or fluoro-methyl; C3-C5 heteroaryl optionally substituted with methyl; and Boc.

In certain preferred embodiments where R¹ is substituted with NR^{m}Rⁿ, R^{m} is H.

In certain preferred embodiments, R^{m} is H and Rⁿ is selected from: H; methyl; ethyl optionally substituted with fluoro or OH; propyl (including isopropyl); cyclopropyl optionally substituted with methyl; cyclobutyl optionally substituted with fluoro; and oxetanyl optionally substituted with methyl or fluoro-methyl.

In certain preferred embodiments, the piperidinyl ring formed by R1 is substituted with NHC(O)R^{m}, wherein R^{m} is selected from H; C1-C3 alkyl optionally substituted with OH or halo (preferably F); C3-C4 cycloalkyl optionally substituted with methyl and/or halo (preferably F); C3-C4 heterocycloalkyl optionally substituted with oxo, methyl or fluoro-methyl; C3-C5 heteroaryl optionally substituted with methyl; and Boc.

In certain preferred embodiments wherein R1 is substituted with NHC(O)R^{m}, R^{m} is selected from C1-C3 alkyl, C3-C4 cycloalkyl, and C4-C5 heteroaryl, for example pyridine.

In certain embodiments, R¹ is NR^{a}R^{b} and R^{a} and R^{b} together with the N to which they are attached form a piperidinyl group optionally substituted with phenyl, fluoro-phenyl, or difluoro-phenyl, and wherein the piperidinyl group is optionally further substituted with NR^{m}Rⁿ,
wherein R^{m} and Rⁿ form a C3-C5 heterocyclyl group together with the N to which they are attached. In certain such embodiments R^{m} and Rⁿ form a morpholinyl group together with the N to which they are attached.

In certain embodiments, R¹ is NR^{a}R^{b} and R^{a} and R^{b} together with the N to which they are attached form a piperidinyl group optionally substituted with phenyl, fluoro-phenyl, or difluoro-phenyl, and wherein the piperidinyl group is optionally further substituted with NH2, NHCH3 or NHCH2CH3.

In certain embodiments wherein the heterocycle formed by R¹ is substituted, it is substituted at the para position (4 position).

In certain embodiments wherein the heterocycle formed by R¹ is substituted, it is substituted at the ortho position (2 position).

In certain such embodiments the heterocycle formed by R¹ is substituted at the ortho position and the para position (2,4 position).

In certain embodiments, R¹ is NR^{a}R^{b} and R^{a} and R^{b} together with the N to which they are attached form a piperidinyl group, wherein the piperidinyl group is substituted at the 4 position with NR^{m}Rⁿ, NHC(O)R^{m}, and NHCH2R¹, and is further substituted at the 2 position with phenyl, fluoro-phenyl, or difluoro-phenyl. In such embodiments, R^{m} and Rⁿ are as defined above and elsewhere herein.

In preferred such embodiments where R¹ is a heterocycle substituted (e.g. by phenyl) at the ortho or 2 position and is chiral, the compound is the (R)-configuration at this position. In preferred embodiments where R¹ is substituted (e.g. by phenyl) at the ortho or 2 position and is chiral, the compound is the (S)-configuration at this position.

In preferred such embodiments where R¹ is a heterocycle substituted (e.g. by NH2 or C1-C2 alkylamino) at the ortho or 2 position and at the para or 4-position and is chiral, the compound is the (R)-configuration at the para position and the (S)-configuration at the ortho position. In preferred such embodiments where R¹ is substituted (e.g. by NH2 or C1-C2 alkylamino) at the ortho or 2 position and at the para or 4-position and is chiral, the compound is the (S)-configuration at the para position and the (R)-configuration at the ortho position.

In certain preferred embodiments, R¹ forms a piperazinyl group substituted with phenyl, fluoro-phenyl, difluoro-phenyl, or thiophenyl. In certain preferred embodiments, R¹ forms a 4-aminopiperidinyl group substituted with phenyl, fluoro-phenyl, difluoro-phenyl, or thiophenyl. Preferably R¹ forms a piperazinyl or 4-aminopiperidinyl group substituted with phenyl. Preferably R¹ forms a piperazinyl or 4-aminopiperidinyl group substituted with fluoro-phenyl. Preferably R¹ forms a piperazinyl or 4-aminopiperidinyl group substituted with difluoro-phenyl.

In preferred embodiments where R¹ is substituted with difluoro-phenyl, the substituent is 2,5 difluoro-phenyl or 3,5 difluoro-phenyl.

In certain embodiments, the piperazinyl or 4-aminopiperidinyl group is optionally further substituted with one or two, preferably one, N-alkyl groups, such as methyl or ethyl.

In certain preferred embodiments, R¹ is:

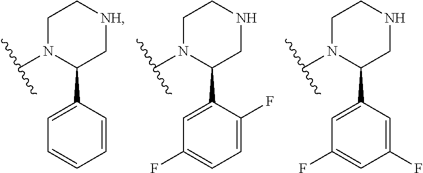 [SMILES: *N1CCNC[C@H]1c1ccccc1 ; *N1CCNC[C@H]1c1cc(F)ccc1F ; *N1CCNC[C@H]1c1cc(F)cc(F)c1]

In certain preferred embodiments, R¹ is:

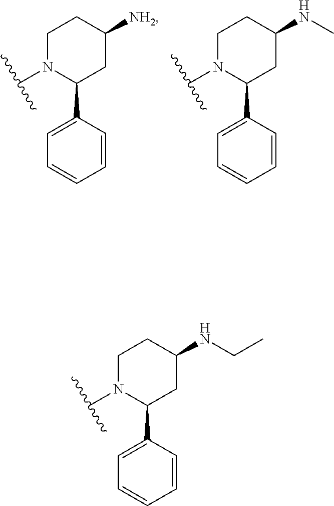 [SMILES: *N1CC[C@@H](N)C[C@H]1c1ccccc1 ; *N1CC[C@@H](NC)C[C@H]1c1ccccc1 ; *N1CC[C@@H](NCC)C[C@H]1c1ccccc1]

In certain preferred embodiments, R¹ is:

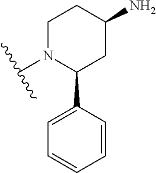 [SMILES: *N1CC[C@@H](N)C[C@H]1c1ccccc1]

In certain preferred embodiments, R¹ is:

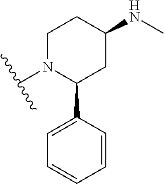 [SMILES: *N1CC[C@@H](NC)C[C@H]1c1ccccc1]

In certain preferred such embodiments, the phenyl ring is mono- or di-substituted with fluoro.

In certain preferred embodiments, R¹ is chosen from:

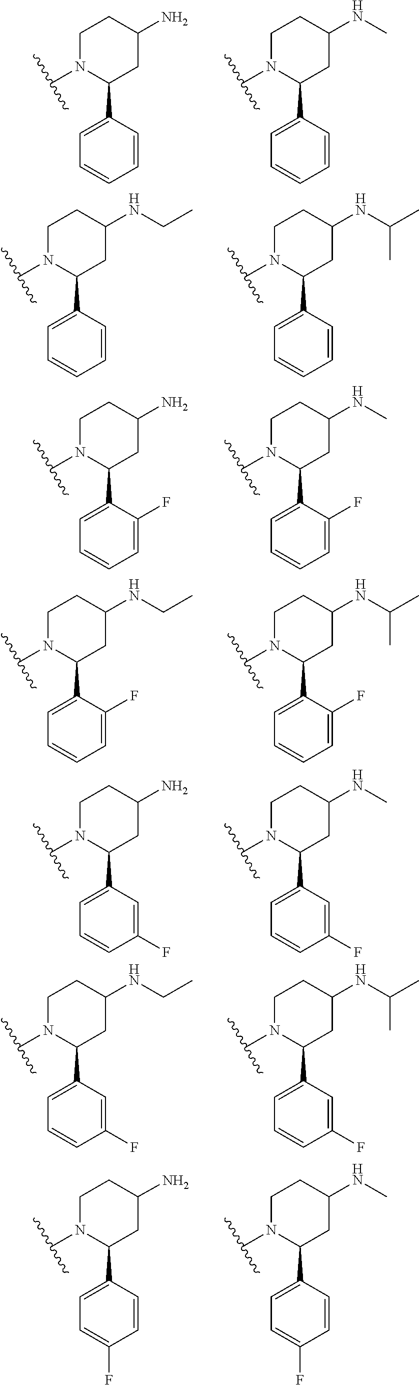

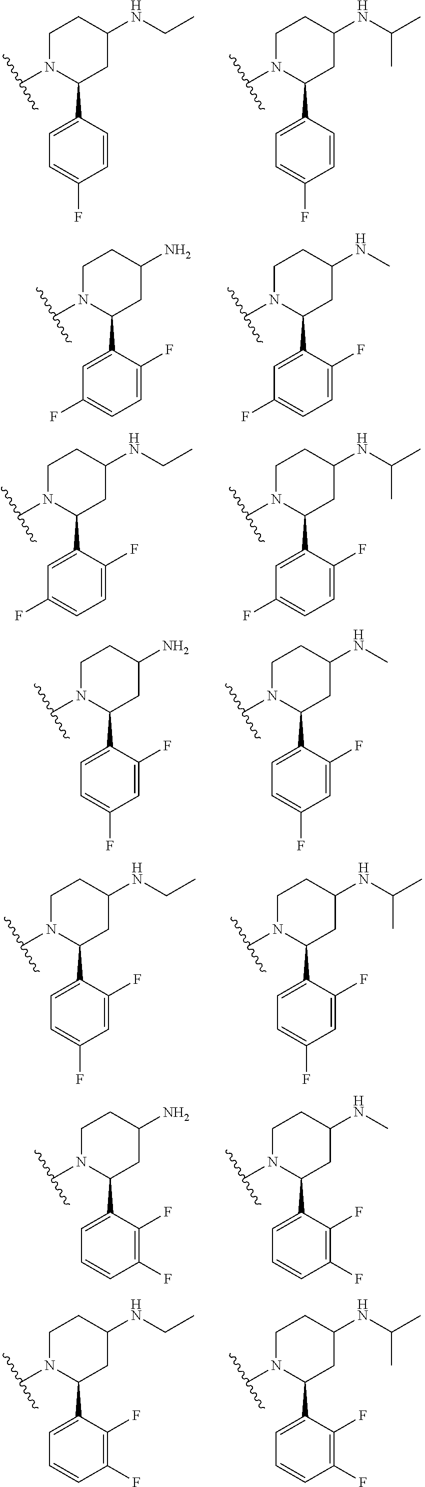

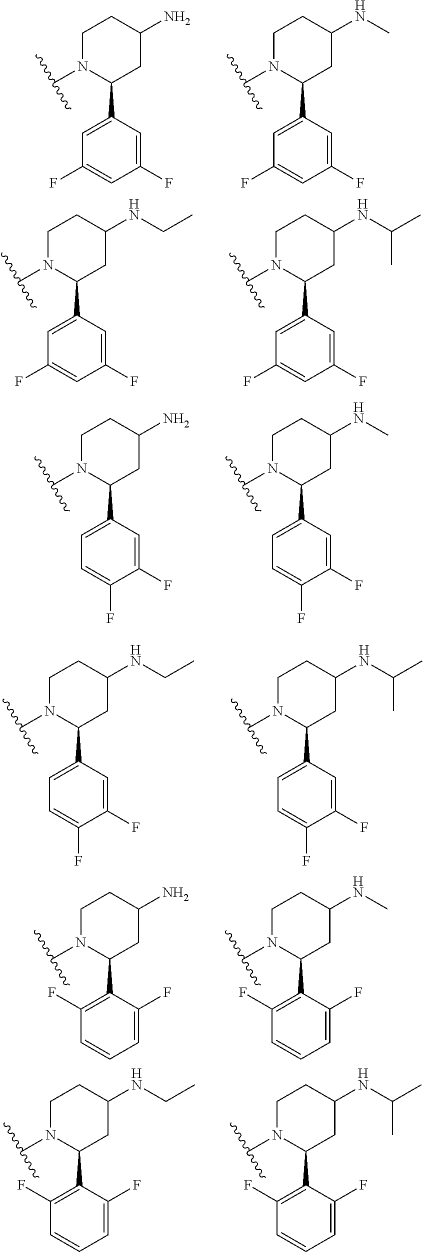

In certain preferred embodiments of the compound of formula (I), R¹ is NR^{a}R^{b} or NR^{a}CH2R^{b}, wherein R^{a} and R^{b} are independently selected from H, methyl, ethyl, propyl, CF3, cyclopropyl, cyclobutyl, cycopentyl, cyclohexyl, phenyl, benzyl, pyridinyl, pyrazole, imidazole, or wherein R^{a} and R^{b} together form a C3-C5 heterocycle together with the N to which they are attached, optionally substituted with OH, CH2OH, CH2OCH3, methyl, ethyl, propyl, CF3, phenyl, or benzyl.

In certain preferred embodiments, R¹ is NR^{a}CH2R^{b}, wherein R^{a} is H or methyl and R^{b} is selected from cyclobutyl optionally substituted with F, cyclohexyl, phenyl optionally substituted with F, furan and thiophene, optionally wherein the methylene group is substituted with CF3.

In certain preferred such embodiments, R^{b} is phenyl or fluoro-substituted phenyl.

In certain preferred embodiments, R₁ forms an optionally substituted C4 or C5 heterocycloalkyl ring linked to the carbonyl of formula (I) via a carbon ring atom, wherein the optional substituent is phenyl. In certain such embodiments the heteroatom in the heterocycloalkyl ring is N.

In certain preferred embodiments of the compound according to formula (I), R² and R³ are independently selected from H, methyl and ethyl, or together form optionally substituted cyclopropyl, optionally substituted cyclobutyl, optionally substituted cyclopentyl, optionally substituted cyclohexyl, optionally substituted pyrrolidine, optionally substituted tetrahydropyran or optionally substituted tetrahydrofuran together with the carbon to which they attached.

In certain such embodiments, R² and R³ are independently selected from H, and methyl. In certain embodiments R² and R³ are both methyl. In certain embodiments R² and R³ are both H.

In certain preferred embodiments, R² and R³ together form cyclohexyl, cyclopentyl, or cyclobutyl together with the carbon to which they attached. Preferably R² and R³ together form cyclopentyl. Alternatively preferably R² and R³ together form cyclohexyl.

In certain embodiments is provided a compound of formula (I), wherein:
X is CR^{4a}, wherein R^{4a} is independently selected from H, optionally substituted C1-C6 alkyl or halo, preferably H or C1-C6 alkyl;
Y is N;
Z is CR⁷, wherein R⁷ is selected from H, halo, C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, COOR⁰, CONR^{c}R^{d}, NR^{c}R^{d}, NS(O)R^{c}R^{d}, S(O)(R^{c})NR^{d}, SOR⁰, SO2R⁰, and SR⁰, wherein R^{c} and R^{d} are independently H, C1-C6 alkyl, C3-C6 cycloalkyl, C5-C6 aryl, C6-C9 arylalkyl, C3-C6 heteroaryl, CN, COOH, or COCH3, or R^{c} and R^{d} together form an optionally substituted C3-C7 heterocycle together with the heteroatom to which they are attached;
M is CH or C—CH3;
and the ring including X, Y and Z is aromatic, and A, D, E and G are all absent.

In certain preferred such embodiments Z is CR⁷ and R⁷ is selected from H, methyl, cyclopropyl, phenyl, pyridine, pyrazole, indazole, imidazole, Cl, Br, COOH, COOCH3, C(O)NR^{c}R^{d}, NR—R^{d}, wherein R^{c}R^{d} are selected from methyl, or wherein R^{c} and R^{d} together form an optionally substituted piperazine, morpholine or optionally substituted pyrrolidine together with the N to which they are attached.

In certain preferred embodiments R⁷ is Cl, Br or C(O)OCH3, or R⁷ is CONR^{c}R^{d} and R^{c} and R^{d} are each methyl, or R^{c} and R^{d} form a piperazinyl ring together with the N to which they are attached.

In certain embodiments,
X is CR^{4a}, wherein R^{4a} is selected from H, optionally substituted C1-C6 alkyl and halo, optionally wherein R^{4a} is H or C1-C6 alkyl;
Y is CR⁵;
Z is N or CR⁷;
M is CH or C—CH3;
wherein the ring including X, Y and Z is aromatic, and A, D, E and G are all absent, and
R⁵ is selected from H, halo, optionally substituted C1-C6 alkyl, C2-C6 alkenyl, C2-C6 alkynyl, optionally substituted C3-C6 cycloalkyl, optionally substituted C5-C8 aryl, optionally substituted C6-C9 arylalkyl, optionally substituted C3-C8 heteroaryl, CH2OH, NR′R″, NS(O)R′R″, SO2R′, C(O)R′, COR′, C(O)OR′, C(O)NR′R″, OR′, and SR′, wherein R′ and R″ are independently selected from H, C1-C6 alkyl, C5-C8 aryl, C6-C9 arylalkyl, and C3-C8 heteroaryl, and
R⁷ is selected from H, halo, cyano, oxo, optionally substituted C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, C(O)OR^{c}, CONR^{c}R^{d}, NR^{c}R^{d}, NS(O)R^{c}R^{d}, S(O)(R^{c})NR^{d}, SOR^{c}, SO2R^{c}, and SR^{c}, wherein R^{c} and R^{d} are independently H, C1-C6 alkyl, C3-C6 cycloalkyl, C5-C6 aryl, C6-C9 arylalkyl, C3-C6 heteroaryl, CN, COOH, or COCH3 or R^{c} and R^{d} together form an optionally substituted C3-C7 heterocycle together with the heteroatom to which they are attached.

In certain preferred embodiments, Z is N or CR⁷ wherein R⁷ is H, C1-C6 alkyl, NR′R″, C(O)NR′R″, cyano, carboxyl, halo, C1-C6 alkylamine, C3-C6 alkylester, optionally substituted C6-C10 aryl or optionally substituted C2-C6 heteroaryl, wherein the one or more heteroatoms are selected from N and O, and the one or more optional substituents of the aryl or heteroaryl are selected from C1-C6 alkyl, C1-C6 alkylamine, amido, and cyano,
wherein R′ and R″ are independently selected from H, C1-C6 alkyl optionally substituted with OH, C3-C7 cycloalkyl, C1-C7 heterocycloalkyl, C4-C7 alkylcycloalkyl, C3-C7 alkylheterocycloalkyl, benzyl, phenyl, and methoxy, or wherein R′ and R″ are joined to one another to form a C2-C7 heterocycle that includes the N to which they are attached, wherein the heterocycle is optionally hydroxyl-substituted, oxo-substituted, methyl-substituted, CH2OH-substituted, or acetyl-substituted.

In certain preferred embodiments, Z is N or CR⁷ wherein R⁷ is selected from: phenyl optionally substituted with amido, cyano or methyl amine; pyridine; oxazole; pyrazole; carboxyl; C(O)NR′R″; or NR′R″;
wherein R′ and R″ are independently selected from H, C1-C6 alkyl, C3-C7 cycloalkyl, C3-C7 heterocycloalkyl wherein the heteroatom is N or O, or wherein R′ and R″ are joined to one another to form a C2-C7 heterocycloalkyl that includes the N to which they are attached, wherein the heterocycloalkyl is optionally hydroxyl-substituted, oxo-substituted, methyl-substituted, CH2OH-substituted, or acetyl-substituted.

In certain preferred embodiments Z is CR⁷ wherein R⁷ is C(O)NR′R″ and wherein R′ and R″ are joined to one another to form an optionally substituted pyrrolidine, piperidine, piperazine or morpholine that includes the N to which they are attached, wherein the piperidine, pyrrolidine, piperazine or morpholine is optionally hydroxyl-substituted, oxo-substituted, methyl-substituted, hydroxymethyl-substituted, or acetyl-substituted.

In certain preferred embodiments, R⁵ is phenyl optionally substituted with one or more substituents independently selected from methyl, halo (e.g. fluoro), and OCH3. In certain preferred embodiments, R⁵ is halo, e.g Cl. In certain preferred embodiments, R⁵ is cyclopropyl optionally substituted with methyl. In certain preferred embodiments, R⁵ is methyl optionally substituted with two or three fluoro. In certain preferred embodiments, R⁵ is SCH3.

In certain preferred such embodiments R^{4a} is H, R⁵ is Cl or phenyl optionally substituted with fluoro, methyl or OCH3, and Z is N or CR⁷.

In certain preferred embodiments, Z is CR⁷ wherein R⁷ is Cl, Br or C(O)OCH3, or R⁷ is CONR^{c}R^{d} and R^{c} and R^{d} are each methyl, or wherein R^{c} and R^{d} form a piperazinyl ring together with the N to which they are attached. In certain preferred such embodiments, R⁷ is di-methyl amide.

In certain preferred embodiments, Z is CR⁷ wherein R⁷ is H.

In certain preferred embodiments, Z is N.

In certain embodiments is provided a compound of formula (I) wherein the ring including X, Y and Z is aromatic, and A, D, E and G are all absent, and wherein:
X is CR^{4a}, wherein R^{4a} is selected from H, optionally substituted C1-C6 alkyl and halo, preferably H or C1-C6 alkyl;
Y is CR⁵;
Z is N or CR⁷;
M is CH or C—CH3,

In certain such embodiments, R^{4a} is H, R⁵ is Cl or phenyl optionally substituted with fluoro, and Z is N or CR⁷.

In certain embodiments:
X is CH
Z is N or CH
M is CH
Y is CR⁵ wherein R⁵ is selected from halo, optionally substituted C1-C6 alkyl, optionally substituted C3-C6 cycloalkyl, optionally substituted C3-C6 heterocycloalkyl, optionally substituted C5-C8 aryl, optionally substituted C3-C8 heteroaryl, and NR′R″, wherein R′ and R″ together form an optionally substituted C3-C6 heterocycloalkyl including the nitrogen to which they are attached.

In certain preferred such embodiments, R⁵ is selected from halo, optionally substituted cyclopropyl, optionally substituted phenyl, optionally substituted thiophenyl, optionally substituted piperidinyl, optionally substituted pyrazolyl, optionally substituted pyrrolidinyl, optionally substituted dihydrobenzofuranyl, optionally substituted azabicyclohexyl, and optionally substituted azetidinyl. Preferably R⁵ is optionally substituted phenyl.

In preferred such embodiments, each of the one or more substituents of R⁵ is selected from the group consisting of: Cl, F, methyl, CHF2, CF3, methoxy, OCHF2, OCF3, and cyclopropyloxy.

In certain embodiments, R⁵ is selected from the group consisting of: halo; phenyl, optionally substituted with fluoro, methoxy or methyl; methyl optionally substituted with fluoro, difluoro or trifluoro; cyclopropyl optionally substituted with methyl.

In certain preferred embodiments, R⁵ is phenyl optionally substituted with F, OCH3 or methyl. In certain preferred embodiments, R⁵ is methyl optionally substituted with fluoro. In certain preferred embodiments, R⁵ is CHF2 or CF3.

In certain preferred embodiments, R⁵ is Cl.

Preferably, in certain such embodiments Z is N. Alternatively preferably, in certain such embodiments Z is CH.

In certain embodiments, X is CR^{4a}, Y is CR⁵; Z is N or CH; M is CH or C—CH3, wherein R^{4a} is H and R⁵ is Cl or phenyl optionally substituted with fluoro or methoxy.

In certain embodiments is provided a compound of formula (I) wherein the ring including X, Y and Z is aliphatic, wherein A, D, E and G are all absent and wherein:
X is absent, CR^{4a}R^{4b}, NR^{4a}, or C═O, wherein R^{4a} and R^{4b} are H, optionally substituted C1-C6 alkyl or halo, or wherein R^{4a} and R^{4b} together form a C3-C6 cycloalkyl or C3-C6 heterocycloalkyl including the carbon to which they are attached;
Y is O, CR⁵R⁶, or NR⁵, wherein R⁵ and R⁶ are independently selected from H, halo, optionally substituted C1-C6 alkyl, optionally substituted C3-C6 cycloalkyl, optionally substituted C5-C8 aryl, optionally substituted C6-C9 arylalkyl, optionally substituted C3-C8 heteroaryl, CH2OH, NR′R″, NS(O)R′R″, COR′, COOR′, C(O)NR′R″, OR′, wherein R′ and R″ are independently selected from C1-C6 alkyl, C5-C8 aryl, C6-C9 arylalkyl, and C3-C8 heteroaryl,
   or wherein R⁵ and R⁶ together form a C3-6 cycloalkyl or C3-C6 heterocycloalkyl including the carbon to which they are attached;
Z is CR⁷R⁸, wherein R⁷ and R⁸ are independently selected from H, halo, C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, COOR⁰, CONR^{c}R^{d}, NR^{c}R^{d}, wherein R^{c} and R^{d} are independently selected from H, C1-C6 alkyl, and C3-C6 cycloalkyl, C5-C6 aryl, C6-C9 arylalkyl, C3-C6 heteroaryl, CN, COOH, or COCH3, or R^{c} and R^{d} together form an optionally substituted C3-C7 heterocycle together with the heteroatom to which they are attached,
or wherein R⁷ and R⁸ together form a C3-6 cycloalkyl or C3-C6 heterocycloalkyl including the carbon to which they are attached;
M is absent, CH2, or Z and M together form part of an optionally substituted phenyl or pyridine ring;
or M is absent and Y and Z together form a fused phenyl or heteroaryl ring,
or M and X are both absent and Z is CHR⁷, wherein R⁷ is selected from H, halo, C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, COOR^{c}, CONR^{c}R^{d}, NR^{c}R^{d}, wherein R^{c} and R^{d} are independently H, C1-C6 alkyl or R^{c} and R^{d} together form an optionally substituted C3-C7 heterocycle together with the heteroatom to which they are attached.

In certain preferred such embodiments:
R^{4a} is selected from H, C1-C6 alkyl or halo, and R^{4b} is H, preferably wherein X is CR^{4a}R^{4b} and R^{4a} is selected from H, and C1-C6 alkyl, and R^{4b} is H;
R⁵ and R⁶ are independently selected from H, halo, optionally substituted C1-C6 alkyl, optionally substituted phenyl, benzyl, pyridinyl, CH2OH, C(O)R′, COR′, C(O)OR′, C(O)NR′R″, and SO2R′, wherein R′ and R″ are independently selected from methyl, ethyl, propyl, butyl, phenyl, and benzyl, or wherein R⁵ and R⁶ together form cyclohexyl, including the carbon to which they are attached, preferably wherein Y is 0 or CR⁵R⁶ and R⁵ and R⁶ are independently selected from H, halo, optionally substituted C1-C6 alkyl, optionally substituted phenyl, benzyl, pyridinyl, CH2OH, C(O)R′, COR′, C(O)OR′, C(O)NR′R″, and SO2R′, wherein R′ and R″ are independently selected from methyl, ethyl, propyl, butyl, phenyl, and benzyl, or wherein R⁵ and R⁶ together form cyclohexyl, including the carbon to which they are attached;
R⁷ is selected from H, C1-C6 alkyl, phenyl, and CONR^{c}R^{d}, wherein R^{c} and R^{d} are independently H, methyl or R^{c} and R^{d} together form an optionally substituted pyrrolidine together with the nitrogen to which they are attached, and R⁸ is H, preferably wherein Z is CR⁷R⁸ and R⁷ is selected from H, C1-C6 alkyl, phenyl, and CONR^{c}R^{d}, wherein R^{c} and R^{d} are independently H, methyl or R^{c} and R^{d} together form an optionally substituted pyrrolidine together with the nitrogen to which they are attached, and R⁸ is H.

In certain embodiments, Z is CH2 and Y is NR⁵. In certain such embodiments, R⁵ is C(O)CH3.

In certain preferred embodiments:
X is CR^{4a}R^{4b} and R^{4a} and R^{4b} are both H;
Y is O or CR⁵R⁶, wherein R⁵ is phenyl or C(O)NR′R″, wherein R′ and R″ are both methyl, and R⁶ is H; and
Z is CR⁷R⁸, wherein R⁷ is phenyl or C(O)NR^{c}R^{d}, wherein R^{c} and R^{d} are both methyl. In certain such embodiments, R⁸ is H.

In certain preferred such embodiments:
X is CR^{4a}R^{4b} and R^{4a} and R^{4b} are both H;
Y is O; and
Z is CR⁷R⁸, wherein R⁷ and R⁸ are both H.

In certain preferred embodiments, X is CR^{4a}R^{4b} and R^{4a} and R^{4b} are both H; Y is N and Z is C and Y and Z together form a fused heteroaryl ring, optionally together form a fused imadozolyl ring.

In certain embodiments, Z is C R⁷R⁸ and Y is NR⁵. In certain preferred such embodiments R⁵ is phenyl, pyridinyl, butyl carboxylate or C(O)CH3, preferably wherein R⁵ is phenyl. In certain preferred such embodiments, Z is CH2.

In certain preferred embodiments of the compound of formula (I) the ring including X, Y and Z is aliphatic, and:
A, D, E and G are each C or N and form a fused aryl or heteroaryl ring with the aliphatic ring including X, Y and Z in the case of a 5-membered ring (where M is absent) and X, Y, Z and M in the case of a 6-membered ring,
X is C
Y is C
Z is NR⁷, CR⁷R⁸ or C═O, wherein R⁷ and R⁸ are independently selected from H, halo, C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, COOR⁰, CONR^{c}R^{d}, NR^{c}R^{d}, NS(O)R^{c}R^{d}, S(O)(R^{c})NR^{d}, SOR^{c}, SO2R^{c}, and SR^{c}, wherein R^{c} and R^{d} are independently H, C1-C6 alkyl, C3-C6 cycloalkyl, C5-C6 aryl, C6-C9 arylalkyl, C3-C6 heteroaryl, CN, COOH, or COCH3, or R^{c} and R^{d} together form an optionally substituted C3-C7 heterocycle together with the heteroatom to which they are attached,
   or wherein R⁷ and R⁸ together form a C3-6 cycloalkyl or C3-C6 heterocycloalkyl including the carbon to which they are attached; and
M is absent or CR¹³R¹⁴, wherein R¹³ and R¹⁴ are independently selected from H, and C1-C6 alkyl, or wherein R¹³ and R¹⁴ together form a C3-C6 cycloalkyl together with the carbon to which they are attached.

In certain preferred such embodiments M is absent and Z is CR⁷R⁸ and wherein R⁷ and R⁸ are H.

In certain preferred such embodiments, A, D and E are C, and G is C or N.

In certain preferred embodiments of the compound of formula (I):
X is C or N
Y is C or N
Z is N, or CR⁷, wherein R⁷ is selected from H, halo, C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, COOR^{c}, CONR^{c}R^{d}, NR^{c}R^{d}, NS(O)R^{c}R^{d}, S(O)(R^{c})NR^{d}, SOR^{c}, SO2R¹, and SR^{c}, wherein R^{c} and R^{d} are independently H, C1-C6 alkyl, C3-C6 cycloalkyl, C5-C6 aryl, C6-C9 arylalkyl, C3-C6 heteroaryl, CN, COOH, or COCH3, or R^{c} and R^{d} together form an optionally substituted C3-C7 heterocycle together with the heteroatom to which they are attached;
M is absent, CH or C—CH3,
A is CR⁹, CHR⁹, N, NR⁹, S, or O,
D is CR⁹, CHR⁹, N or NR⁹,
G is absent, CR⁹, CHR⁹, or N,
   wherein R⁹ is independently selected from H, halo, C1-C6 alkyl, CF3, and OR*, wherein R* is an optionally substituted C1-C6 alkyl, optionally substituted C1-C6 cycloalkyl or optionally substituted heterocycloalkyl, and
E is CR¹⁰, CHR¹⁰, N, NR¹⁰, S, or O,
   wherein R¹⁰ is selected from H, halo, C1-C6 alkyl, C3-C6 cycloalkyl, C5-C8 aryl, C6-C9 arylalkyl, C4-C8 heteroaryl, SR^{x}, OR^{x}, NR^{x}R^{y}, and NS(O)R^{x}R^{y}, S(O)(R^{x})NR^{y}, wherein R^{x} and R^{y} are independently selected from H, C1-C6 alkyl, CF3, C3-C6 cycloalkyl, C5-C8 aryl, C6-C9 arylalkyl, C4-C8 heteroaryl, COOH, amido, cyano, C2-C6 alkene, C2-C6 alkyne, or wherein R^{x} and R^{y} together form an optionally substituted C4-C6 heterocycloalkyl together with the nitrogen to which they are attached.

In certain preferred such embodiments Z is N, or CR⁷, wherein R⁷ is selected from H, C1-C6 alkyl, CN or C(O)NR^{c}R^{d}, wherein R^{c} and R^{d} are independently H, methyl, or together form an optionally substituted piperidine, piperazine or morpholine ring together with the nitrogen to which they are attached.

In certain preferred such embodiments, Z is N, or CR⁷, wherein R⁷ is selected from H, C1-C6 alkyl, CN or C(O)NR^{c}R^{d}, wherein R^{c} and R^{d} are independently H, methyl, or together form an optionally substituted piperidine, piperazine or morpholine ring together with the nitrogen to which they are attached.

In certain preferred such embodiments:
E is CR¹⁰, CHR¹⁰, N, NR¹⁰, S, or O,
wherein R¹⁰ is selected from H, F, Cl, Br, methyl, ethyl, cyclopropyl, cyclobutyl, cyclopentyl, cyclohexyl, SR^{x}, OR^{x}, NR^{x}R^{y}, and NS(O)(CH3)2, wherein R^{x} and R^{y} are independently selected from H, methyl, ethyl, CF3, cyclopropyl, cyclobutyl, cyclopentyl, cyclohexyl, COOH, amido, cyano, or wherein R^{x} and R^{y} together form a piperidine, piperazine or morpholine together with the nitrogen to which they are attached, optionally substituted with methyl.

In certain preferred embodiments:
A, M, X and Y are C, E is CR¹⁰,
D is N,
G is C or N, and
Z is C or N,
such that the rings including A, D, E, G, X, Y, Z and M form a fused aromatic ring system. In certain preferred such embodiments, G is C and Z is C.

In certain preferred embodiments:
M is absent,
A, X and Y are C, D and G are N,
E is CR¹⁰, and Z is NR⁷,
and the ring including A, D, E, G, X, and Y, forms an aromatic ring fused to the ring including X, Y and Z,
wherein R⁷ is H or C1-C6 alkyl, optionally wherein R⁷ is methyl.

In certain preferred embodiments:
M, A, X, Y and Z are C,
D and G are N,
E is CR¹⁰,
and the ring including A, D, E, G, X, and Y, forms an aromatic ring fused to the ring including X, Y, M and Z.

In certain preferred embodiments E is CR¹⁰, wherein R¹⁰ is H or SR^{x}, wherein R^{x} is C1-C6 alkyl. Preferably R^{x} is methyl.

In certain preferred embodiments:
X, Y, M, A and G are C,
Z is N, D is CR⁹ and E is CR¹⁰,
such that the rings including A, D, E, G, X, Y, Z and M form a fused aromatic ring system, wherein R⁹ is halo, preferably F or Cl, and R¹⁰ is H or halo, optionally F or Cl.

In certain preferred embodiments:
G is absent, A is C, D and Z are N, and E is NR¹⁰,
   such that the rings including A, D, E, X, Y, Z and M form a fused aromatic ring system, wherein R¹⁰ is selected from H, ethyl, cyclopropyl, phenyl and benzyl. In a preferred embodiment R¹⁰ is cyclopropyl.

In preferred such embodiments, R² is not H, and R³ is not H. This embodiment is particularly advantageous because it improves selectivity for USP19 inhibition compared to other USPs. In certain preferred such embodiments, R² and R³ are both CH3, or together form a C3-C6 cycloalkyl together with the carbon to which they are attached. In certain preferred embodiments, R² and R³ form cyclopentyl together with the carbon to which they are attached.

In certain preferred embodiments of the compound of formula (I):
X is CR⁴, wherein R⁴ is independently selected from H, C1-C6 alkyl or halo;
Y is CR⁵, wherein R⁵ is selected from H, halo, C1-C6 alkyl, C3-C6 cycloalkyl, optionally halo-substituted phenyl, optionally halo-substituted benzyl, pyridinyl, pyrazole, imidazole, CH2OH, NR′R″, COR′, C(O)OR′, C(O)NR′R″, OR′, wherein R′ and R″ are independently selected from C1-C6 alkyl, and phenyl, benzyl, pyridinyl, pyrazole, imidazole;
Z is CR⁷, wherein R⁷ is selected from H, halo, C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, COOR^{c}, CONR^{c}R^{d}, NR^{c}R^{d}, NS(O)R^{c}R^{d}, S(O)(R^{c})NR^{d}, SOR^{c}, SO2R¹, and SR^{c}, wherein R^{c} and R^{d} are independently H, C1-C6 alkyl, C3-C6 cycloalkyl, C5-C6 aryl, C6-C9 arylalkyl, C3-C6 heteroaryl, CN, COOH, or COCH3, or R^{c} and R^{d} together form an optionally substituted C3-C7 heterocycle together with the heteroatom to which they are attached;
M is CH; and the ring including X, Y and Z is aromatic, and A, D, E and G are all absent.

In certain preferred embodiments, Z is CH.

In certain preferred embodiments, R⁵ is phenyl optionally substituted with F, OCH3 or methyl. In certain preferred embodiments, R⁵ is methyl optionally substituted with fluoro. In certain preferred embodiments, R⁵ is CHF2 or CF3.

In certain embodiments of the compounds provided herein, the compound is chiral at the tertiary alcohol position of Formula (I). In preferred embodiments, the compound is in the (R)-configuration. In alternative preferred embodiments, the compound is in the (S)-configuration.

In certain preferred embodiments:
X is CR^{4a}R^{4b}, wherein R^{4a} and R^{4b} are independently selected from H, optionally substituted C1-C6 alkyl and halo; or wherein R^{4a} and R^{4b} together form a C3-C6 cycloalkyl or C3-C6 heterocycloalkyl including the carbon to which they are attached;
Y is O,
Z is CR⁷R⁸, wherein R⁷ and R⁸ are independently selected from H, halo, cyano, oxo, optionally substituted C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, C(O)OR^{c}, CONR^{c}R^{d}, NR^{c}R^{d}, NS(O)R^{c}R^{d}, S(O)(R^{c})NR^{d}, SOR^{c}, SO2R^{c}, and SR^{c}, wherein R^{c} and R^{d} are independently H, C1-C6 alkyl, C3-C6 cycloalkyl, C5-C6 aryl, C6-C9 arylalkyl, C3-C6 heteroaryl, CN, COOH, or COCH3 or R^{c} and R^{d} together form an optionally substituted C3-C7 heterocycle together with the heteroatom to which they are attached; or wherein R⁷ and R⁸ together form a C3-6 cycloalkyl or C3-C6 heterocycloalkyl including the carbon to which they are attached;
M is CR¹³R¹⁴, wherein R¹³ and R¹⁴ are independently selected from H, and C1-C6 alkyl, or wherein R¹³ and R¹⁴ together form a C3-C6 cycloalkyl or C3-C6 heterocycloalkyl together with the carbon to which they are attached;
wherein the ring including XYZM is aliphatic.

In certain preferred such embodiments, X, Z and M are CH2 and Y is O.

In certain embodiments is provided a compound selected from:
(R)-6-Phenyl-3-((1-(2-phenylpiperazine-1-carbonyl)piperidin-4-yl)methyl)pyrimidin-4(3H)-one
(R)-6-Phenyl-3-((7-(2-phenylpiperazine-1-carbonyl)-7-azaspiro[4.5]dec-9-en-10-yl)methyl)pyrimidin-4(3H)-one
(R,Z)-6-phenyl-3-((7-(2-phenylpiperazine-1-carbonyl)-7-azaspiro[4.5]decan-10-ylidene)methyl)pyrimidin-4(3H)-one
6-Phenyl-3-((7-((R)-4,4,4-trifluoro-2-methylbutanoyl)-7-azaspiro[4.5]decan-10-yl)methyl)pyrimidin-4(3H)-one
4-Phenyl-1-((7-((R)-4,4,4-trifluoro-2-methylbutanoyl)-7-azaspiro[4.5]decan-10-yl)methyl)pyridin-2(1H)-one
(R)-4-Phenyl-1-((1-(2-phenylpiperazine-1-carbonyl)piperidin-4-yl)methyl)pyridin-2(1H)-one
6-Phenyl-3-((7-((S)-4,4,4-trifluoro-2-(methoxymethyl)butanoyl)-7-azaspiro[4.5]decan-10-yl)methyl)pyrimidin-4(3H)-one
6-Phenyl-3-((7-((S)-2-phenylpyrrolidine-1-carbonyl)-7-azaspiro[4.5]decan-10-yl)methyl)pyrimidin-4(3H)-one
3-((1-((2S,4R)-4-Amino-2-phenylpiperidine-1-carbonyl)piperidin-4-yl)methyl)-6-phenylpyrimidin-4(3H)-one
N-Benzyl-10-((6-oxo-4-phenylpyrimidin-1(6H)-yl)methyl)-7-azaspiro[4.5]decane-7-carboxamide
3-((7-((R)-2-(3,5-Difluorophenyl)piperazine-1-carbonyl)-7-azaspiro[4.5]decan-10-yl)methyl)-6-phenylpyrimidin-4(3H)-one
3-((7-((3R,4R)-1-Imino-1-oxido-4-phenyltetrahydro-1H-1λ⁶-thiophene-3-carbonyl)-7-azaspiro[4.5]decan-10-yl)methyl)-6-phenylpyrimidin-4(3H)-one
3-((7-((3S,4S)-1-Imino-1-oxido-4-phenyltetrahydro-1H-1λ⁶-thiophene-3-carbonyl)-7-azaspiro[4.5]decan-10-yl)methyl)-6-phenylpyrimidin-4(3H)-one
6-Phenyl-3-(((S)-7-((R)-2-phenylpiperazine-1-carbonyl)-7-azaspiro[4.5]decan-10-yl)methyl)pyrimidin-4(3H)-one
6-Phenyl-3-(((R)-7-((R)-2-phenylpiperazine-1-carbonyl)-7-azaspiro[4.5]decan-10-yl)methyl)pyrimidin-4(3H)-one
4-Phenyl-1-(((S)-7-((R)-2-phenylpiperazine-1-carbonyl)-7-azaspiro[4.5]decan-10-yl)methyl)pyridin-2(1H)-one
4-Phenyl-1-(((R)-7-((R)-2-phenylpiperazine-1-carbonyl)-7-azaspiro[4.5]decan-10-yl)methyl)pyridin-2(1H)-one
1-((1-((2S,4R)-4-Amino-2-phenylpiperidine-1-carbonyl)piperidin-4-yl)methyl)-4-phenylpyridin-2(1H)-one
3-((3,3-Dimethyl-1-((R)-2-phenylpiperazine-1-carbonyl)piperidin-4-yl)methyl)-6-phenylpyrimidin-4(3H)-one
3-((1-((2S,4R)-4-Amino-2-phenylpiperidine-1-carbonyl)-3,3-dimethylpiperidin-4-yl)methyl)-6-phenylpyrimidin-4(3H)-one
1-((3,3-Dimethyl-1-((R)-2-phenylpiperazine-1-carbonyl)piperidin-4-yl)methyl)-4-phenylpyridin-2(1H)-one
1-((1-((2S,4R)-4-Amino-2-phenylpiperidine-1-carbonyl)-3,3-dimethylpiperidin-4-yl)methyl)-4-phenylpyridin-2(1H)-one
(R)-1-((1-(2-(2,5-Difluorophenyl)piperazine-1-carbonyl)piperidin-4-yl)methyl)-4-phenylpyridin-2(1H)-one
1-(((R)-7-((2S,4R)-4-Amino-2-phenylpiperidine-1-carbonyl)-7-azaspiro[4.5]decan-10-yl)methyl)-4-phenylpyridin-2(1H)-one
1-(((S)-7-((2S,4R)-4-Amino-2-phenylpiperidine-1-carbonyl)-7-azaspiro[4.5]decan-10-yl)methyl)-4-phenylpyridin-2(1H)-one
3-(((R)-7-((2S,4R)-4-Amino-2-phenylpiperidine-1-carbonyl)-7-azaspiro[4.5]decan-10-yl)methyl)-6-phenylpyrimidin-4(3H)-one
3-(((S)-7-((2S,4R)-4-Amino-2-phenylpiperidine-1-carbonyl)-7-azaspiro[4.5]decan-10-yl)methyl)-6-phenylpyrimidin-4(3H)-one
3-((7-((2S,4R)-4-Amino-2-phenylpiperidine-1-carbonyl)-7-azaspiro[4.5]decan-10-yl)methyl)-6-fluoroquinazolin-4(3H)-one
3-((7-((2S,4R)-4-Amino-2-phenylpiperidine-1-carbonyl)-7-azaspiro[4.5]decan-10-yl)methyl)-6-(difluoromethyl)pyrimidin-4(3H)-one
3-((7-((2S,4R)-4-Amino-2-phenylpiperidine-1-carbonyl)-7-azaspiro[4.5]decan-10-yl)methyl)-6-cyclopropylpyrimidin-4(3H)-one
3-((7-((2S,4R)-4-Amino-2-phenylpiperidine-1-carbonyl)-7-azaspiro[4.5]decan-10-yl)methyl)pyrimidin-4(3H)-one
1-((7-((2S,4R)-4-Amino-2-phenylpiperidine-1-carbonyl)-7-azaspiro[4.5]decan-10-yl)methyl)pyrazin-2(1H)-one
3-((7-((2S,4R)-4-Amino-2-phenylpiperidine-1-carbonyl)-7-azaspiro[4.5]decan-10-yl)methyl)-6-methylpyrimidin-4(3H)-one
2-((7-((2S,4R)-4-Amino-2-phenylpiperidine-1-carbonyl)-7-azaspiro[4.5]decan-10-yl)methyl)-2,7-naphthyridin-1(2H)-one
3-((7-((2S,4R)-4-Amino-2-phenylpiperidine-1-carbonyl)-7-azaspiro[4.5]decan-10-yl)methyl)-6-(trifluoromethyl)pyrimidin-4(3H)-one
5-((7-((2S,4R)-4-Amino-2-phenylpiperidine-1-carbonyl)-7-azaspiro[4.5]decan-10-yl)methyl)-1-cyclopropyl-1,5-dihydro-4H-pyrazolo[3,4-d]pyrimidin-4-one
3-((7-((2S,4R)-4-Amino-2-phenylpiperidine-1-carbonyl)-7-azaspiro[4.5]decan-10-yl)methyl)-6-(1-methylcyclopropyl)pyrimidin-4(3H)-one
3-((7-((2S,4R)-4-Amino-2-phenylpiperidine-1-carbonyl)-7-azaspiro[4.5]decan-10-yl)methyl)-6,7-difluoroquinazolin-4(3H)-one
1-((7-((2S,4R)-4-Amino-2-phenylpiperidine-1-carbonyl)-7-azaspiro[4.5]decan-10-yl)methyl)-4-(difluoromethyl)pyridin-2(1H)-one
1-((7-((2S,4R)-4-Amino-2-phenylpiperidine-1-carbonyl)-7-azaspiro[4.5]decan-10-yl)methyl)-4-methylpyridin-2(1H)-one
6-((7-((2S,4R)-4-Amino-2-phenylpiperidine-1-carbonyl)-7-azaspiro[4.5]decan-10-yl)methyl)-2-(methylthio)pyrido[4,3-d]pyrimidin-5(6H)-one
4-((7-((2S,4R)-4-Amino-2-phenylpiperidine-1-carbonyl)-7-azaspiro[4.5]decan-10-yl)methyl)morpholin-3-one
7-((7-((2S,4R)-4-Amino-2-phenylpiperidine-1-carbonyl)-7-azaspiro[4.5]decan-10-yl)methyl)-7,8-dihydroimidazo[1,2-a]pyrazin-6(5H)-one
1-((7-((2S,4R)-4-Amino-2-phenylpiperidine-1-carbonyl)-7-azaspiro[4.5]decan-10-yl)methyl)-4-(trifluoromethyl)pyridin-2(1H)-one
1-(((R)-7-((2S,4R)-4-Hydroxy-2-phenylpiperidine-1-carbonyl)-7-azaspiro[4.5]decan-10-yl)methyl)-4-phenylpyridin-2(1H)-one
1-(((R)-7-((2S,4R)-4-(Ethylamino)-2-phenylpiperidine-1-carbonyl)-7-azaspiro[4.5]decan-10-yl)methyl)-4-phenylpyridin-2(1H)-one
1-(((R)-7-((2S,4R)-4-(Dimethylamino)-2-phenylpiperidine-1-carbonyl)-7-azaspiro[4.5]decan-10-yl)methyl)-4-phenylpyridin-2(1H)-one
1-((1-((2S,4R)-4-(Methylamino)-2-phenylpiperidine-1-carbonyl)piperidin-4-yl)methyl)-4-phenylpyridin-2(1H-one
1-(((R)-7-((2S,4R)-4-(Methylamino)-2-phenylpiperidine-1-carbonyl)-7-azaspiro[4.5]decan-10-yl)methyl)-4-phenylpyridin-2(1H)-one
3-((10-Amino-7-((R)-4,4,4-trifluoro-2-methylbutanoyl)-7-azaspiro[4.5]decan-10-yl)methyl)-6-phenylpyrimidin-4(3H)-one
1-(((R)-7-((2S,4R)-4-((1,1-Dioxidothietan-3-yl)amino)-2-phenylpiperidine-1-carbonyl)-7-azaspiro[4.5]decan-10-yl)methyl)-4-phenylpyridin-2(1H)-one
1-(((R)-7-((2S,4S)-4-((1,1-Dioxidothietan-3-yl)amino)-2-phenylpiperidine-1-carbonyl)-7-azaspiro[4.5]decan-10-yl)methyl)-4-phenylpyridin-2(1H)-one
1-(((R)-7-((2S,4R)-4-Hydroxy-4-methyl-2-phenylpiperidine-1-carbonyl)-7-azaspiro[4.5]decan-10-yl)methyl)-4-phenylpyridin-2(1H)-one
4-(2-Methoxyphenyl)-1-(((R)-7-((2S,4R)-4-(methylamino)-2-phenylpiperidine-1-carbonyl)-7-azaspiro[4.5]decan-10-yl)methyl)pyridin-2(1H)-one
1-(((R)-7-((2S,4R)-4-(Methylamino)-2-phenylpiperidine-1-carbonyl)-7-azaspiro[4.5]decan-10-yl)methyl)-4-(o-tolyl)pyridin-2(1H)-one
4-(2-Fluorophenyl)-1-(((R)-7-((2S,4R)-4-(methylamino)-2-phenylpiperidine-1-carbonyl)-7-azaspiro[4.5]decan-10-yl)methyl)pyridin-2(1H)-one
3-(((R)-7-((2S,4R)-4-(Methylamino)-2-phenylpiperidine-1-carbonyl)-7-azaspiro[4.5]decan-10-yl)methyl)-6-phenylpyrimidin-4(3H)-one
3-((7-((2S,4R)-4-Amino-2-phenylpiperidine-1-carbonyl)-10-fluoro-7-azaspiro[4.5]decan-10-yl)methyl)-6-phenylpyrimidin-4(3H)-one
1-(((S)-10-Methoxy-7-((2S,4R)-4-(methylamino)-2-phenylpiperidine-1-carbonyl)-7-azaspiro[4.5]decan-10-yl)methyl)-4-phenylpyridin-2(1H)-one
1-(((R)-7-((2S,4R)-4-Amino-2-phenylpiperidine-1-carbonyl)-7-azaspiro[4.5]decan-10-yl)methyl)-4-(2-methoxyphenyl)pyridin-2(1H)-one
1-(((R)-7-((2S,4R)-4-Amino-2-phenylpiperidine-1-carbonyl)-7-azaspiro[4.5]decan-10-yl)methyl)-4-(o-tolyl)pyridin-2(1H)-one
1-(((R)-7-((2S,4R)-4-Amino-2-phenylpiperidine-1-carbonyl)-7-azaspiro[4.5]decan-10-yl)methyl)-4-(2-fluorophenyl)pyridin-2(1H)-one
3-(((R)-7-((2S,4R)-4-(Ethylamino)-2-phenylpiperidine-1-carbonyl)-7-azaspiro[4.5]decan-10-yl)methyl)-6-phenylpyrimidin-4(3H)-one
3-(((R)-7-((2S,4R)-2-(3-Fluorophenyl)-4-(methylamino)piperidine-1-carbonyl)-7-azaspiro[4.5]decan-10-yl)methyl)-6-phenylpyrimidin-4(31H)-one
1-(((R)-7-((2S,4R)-2-(3-Fluorophenyl)-4-(methylamino)piperidine-1-carbonyl)-7-azaspiro[4.5]decan-10-yl)methyl)-4-phenylpyridin-2(1H)-one
1-(((R)-7-((2S,4R)-4-Amino-2-(3-fluorophenyl)piperidine-1-carbonyl)-7-azaspiro[4.5]decan-10-yl)methyl)-4-phenylpyridin-2(1H)-one
3-(((R)-7-((2S,4R)-4-Amino-2-(3-fluorophenyl)piperidine-1-carbonyl)-7-azaspiro[4.5]decan-10-yl)methyl)-6-phenylpyrimidin-4(3H)-one
1-(((R)-7-((2S,4R)-4-Amino-2-phenylpiperidine-1-carbonyl)-7-azaspiro[4.5]decan-10-yl)methyl)-4-chloropyridin-2(1H)-one
1-(((R)-7-((2R,4R)-4-Amino-2-ethylpiperidine-1-carbonyl)-7-azaspiro[4.5]decan-10-yl)methyl)-4-phenylpyridin-2(1H)-one
1-(((R)-1-((2S,4R)-4-Amino-2-phenylpiperidine-1-carbonyl)-3,3-dimethylpiperidin-4-yl)methyl)-4-phenylpyridin-2(1H)-one
3-(((R)-7-((2S,4S)-4-Amino-2-phenylpyrrolidine-1-carbonyl)-7-azaspiro[4.5]decan-10-yl)methyl)-6-phenylpyrimidin-4(3H)-one
3-(((R)-7-((2S,4R)-4-((2,2-Difluoroethyl)amino)-2-phenylpiperidine-1-carbonyl)-7-azaspiro[4.5]decan-10-yl)methyl)-6-phenylpyrimidin-4(31H)-one
1-(((R)-3,3-Dimethyl-1-((2S,4R)-4-(methylamino)-2-phenylpiperidine-1-carbonyl)piperidin-4-yl)methyl)-4-phenylpyridin-2(1H)-one
1-(((S)-7-((2S,4R)-4-Amino-2-phenylpiperidine-1-carbonyl)-10-methoxy-7-azaspiro[4.5]decan-10-yl)methyl)-4-phenylpyridin-2(1H)-one
3-((1-((2S,4R)-2-(3-Fluorophenyl)-4-(methylamino)piperidine-1-carbonyl)piperidin-4-yl)methyl)-6-phenylpyrimidin-4(3H)-one
3-((1-((2S,4R)-4-(Ethylamino)-2-phenylpiperidine-1-carbonyl)piperidin-4-yl)methyl)-6-phenylpyrimidin-4(3H)-one
Methyl (2S,4R)-1-((R)-10-((6-oxo-4-phenylpyrimidin-1(6H)-yl)methyl)-7-azaspiro[4.5]decane-7-carbonyl)-2-phenylpiperidine-4-carboxylate
3-(((R)-7-((2S,4R)-4-Amino-2-phenylpiperidine-1-carbonyl)-7-azaspiro[4.5]decan-10-yl)methyl)-6-(o-tolyl)pyrimidin-4(3H)-one
3-(((R)-7-((2S,4R)-2-(2,5-Difluorophenyl)-4-(methylamino)piperidine-1-carbonyl)-7-azaspiro[4.5]decan-10-yl)methyl)-6-phenylpyrimidin-4(3H)-one
3-(((R)-7-((2S,4R)-4-(Cyclopropylamino)-2-phenylpiperidine-1-carbonyl)-7-azaspiro[4.5]decan-10-yl)methyl)-6-phenylpyrimidin-4(3H)-one
3-(((R)-7-((2S,4R)-4-Amino-2-(2,4-difluorophenyl)piperidine-1-carbonyl)-7-azaspiro[4.5]decan-10-yl)methyl)-6-phenylpyrimidin-4(3H)-one
3-(((R)-7-((2S,4R)-4-Amino-2-(3-fluorophenyl)piperidine-1-carbonyl)-7-azaspiro[4.5]decan-10-yl)methyl)-6-(o-tolyl)pyrimidin-4(3H)-one
3-(((R)-7-((2S,4R)-4-Amino-2-(2,5-difluorophenyl)piperidine-1-carbonyl)-7-azaspiro[4.5]decan-10-yl)methyl)-6-(o-tolyl)pyrimidin-4(3H)-one
4-Chloro-1-(((R)-7-((2S,4R)-4-(methylamino)-2-phenylpiperidine-1-carbonyl)-7-azaspiro[4.5]decan-10-yl)methyl)pyridin-2(1H)-one
3-(((R)-7-((2S,4R)-4-(Methylamino)-2-phenylpiperidine-1-carbonyl)-7-azaspiro[4.5]decan-10-yl)methyl)-6-(o-tolyl)pyrimidin-4(3H)-one
1-(((R)-7-((2S,4S,5R)-4-Amino-5-fluoro-2-phenylpiperidine-1-carbonyl)-7-azaspiro[4.5]decan-10-yl)methyl)-4-phenylpyridin-2(1H)-one
1-(((R)-7-((2S,4S,5S)-4-Amino-5-fluoro-2-phenylpiperidine-1-carbonyl)-7-azaspiro[4.5]decan-10-yl)methyl)-4-phenylpyridin-2(1H)-one
3-(((R)-7-((2S,4R)-4-Amino-2-(2,5-difluorophenyl)piperidine-1-carbonyl)-7-azaspiro[4.5]decan-10-yl)methyl)-6-phenylpyrimidin-4(3H)-one
1-(((R)-7-((2S,4R)-4-Amino-2-(2,5-difluorophenyl)piperidine-1-carbonyl)-7-azaspiro[4.5]decan-10-yl)methyl)-4-phenylpyridin-2(1H)-one
1-(((R)-7-((2S,4R)-4-Amino-2-(3-fluorophenyl)piperidine-1-carbonyl)-7-azaspiro[4.5]decan-10-yl)methyl)-4-(2-methoxyphenyl)pyridin-2(1H)-one
1-(((R)-7-((2S,4R)-4-Amino-2-(2,5-difluorophenyl)piperidine-1-carbonyl)-7-azaspiro[4.5]decan-10-yl)methyl)-4-(2-methoxyphenyl)pyridin-2(1H)-one
3-(((R)-7-((2S,4S)-4-(Methylamino)-2-phenylpyrrolidine-1-carbonyl)-7-azaspiro[4.5]decan-10-yl)methyl)-6-phenylpyrimidin-4(3H)-one
1-(((R)-7-((2S,4R)-2-(2,5-Difluorophenyl)-4-(methylamino)piperidine-1-carbonyl)-7-azaspiro[4.5]decan-10-yl)methyl)-4-phenylpyridin-2(1H)-one
3-(((R)-7-((2S,4R)-4-Amino-2-phenylpiperidine-1-carbonyl)-7-azaspiro[4.5]decan-10-yl)methyl)-6-(2-methoxyphenyl)pyrimidin-4(3H)-one
N-((2S,4R)-1-((R)-10-((2-Oxo-4-phenylpyridin-1(2H)-yl)methyl)-7-azaspiro[4.5]decane-7-carbonyl)-2-phenylpiperidin-4-yl)acetamide
3-(((R)-7-((2S,4R)-2-(2,4-Difluorophenyl)-4-(methylamino)piperidine-1-carbonyl)-7-azaspiro[4.5]decan-10-yl)methyl)-6-phenylpyrimidin-4(3H)-one
1-(((R)-7-((2S,4R)-2-(2,4-Difluorophenyl)-4-(methylamino)piperidine-1-carbonyl)-7-azaspiro[4.5]decan-10-yl)methyl)-4-phenylpyridin-2(1H)-one
N-((2S,4R)-1-((R)-10-((2-Oxo-4-phenylpyridin-1(2H)-yl)methyl)-7-azaspiro[4.5]decane-7-carbonyl)-2-phenylpiperidin-4-yl)cyclopropanecarboxamide
6-(2-Methoxyphenyl)-3-(((R)-7-((2S,4R)-4-(methylamino)-2-phenylpiperidine-1-carbonyl)-7-azaspiro[4.5]decan-10-yl)methyl)pyrimidin-4(3H)-one
1-((1-((2S,4R)-2-(2,5-Difluorophenyl)-4-(methylamino)piperidine-1-carbonyl)piperidin-4-yl)methyl)-4-phenylpyridin-2(1H)-one
3-(((R)-7-((2S,4R)-2-(2,5-Difluorophenyl)-4-(methylamino)piperidine-1-carbonyl)-7-azaspiro[4.5]decan-10-yl)methyl)-6-(2-methoxyphenyl)pyrimidin-4(3H)-one
3-(((R)-7-((2S,4R)-4-Amino-2-phenylpiperidine-1-carbonyl)-7-azaspiro[4.5]decan-10-yl)methyl)-6-fluoroquinazolin-4(3H)-one
6-Fluoro-3-(((R)-7-((2S,4R)-4-(methylamino)-2-phenylpiperidine-1-carbonyl)-7-azaspiro[4.5]decan-10-yl)methyl)quinazolin-4(3H)-one
3-(((R)-7-((2S,4R)-2-(3-Fluorophenyl)-4-(methylamino)piperidine-1-carbonyl)-7-azaspiro[4.5]decan-10-yl)methyl)-6-(2-methoxyphenyl)pyrimidin-4(3H)-one
3-(((R)-7-((2S,4R)-2-(2-Fluorophenyl)-4-(methylamino)piperidine-1-carbonyl)-7-azaspiro[4.5]decan-10-yl)methyl)-6-(2-methoxyphenyl)pyrimidin-4(3H)-one
3-(((R)-7-((2S,4R)-2-(4-Fluorophenyl)-4-(methylamino)piperidine-1-carbonyl)-7-azaspiro[4.5]decan-10-yl)methyl)-6-(2-methoxyphenyl)pyrimidin-4(3H)-one
Methyl ((2S,4R)-1-((R)-10-((6-oxo-4-phenylpyrimidin-1(6H)-yl)methyl)-7-azaspiro[4.5]decane-7-carbonyl)-2-phenylpiperidin-4-yl)glycinate
Sodium ((2S,4R)-1-((R)-10-((6-oxo-4-phenylpyrimidin-1(6H)-yl)methyl)-7-azaspiro[4.5]decane-7-carbonyl)-2-phenylpiperidin-4-yl)glycinate
3-(((R)-7-((2S,4R)-4-Amino-2-(3,5-difluorophenyl)piperidine-1-carbonyl)-7-azaspiro[4.5]decan-10-yl)methyl)-6-phenylpyrimidin-4(3H)-one
1-(((R)-7-((2S,4R)-4-Amino-2-(3,5-difluorophenyl)piperidine-1-carbonyl)-7-azaspiro[4.5]decan-10-yl)methyl)-4-phenylpyridin-2(1H)-one
6-(2-Fluorophenyl)-3-(((R)-7-((2S,4R)-4-(methylamino)-2-phenylpiperidine-1-carbonyl)-7-azaspiro[4.5]decan-10-yl)methyl)pyrimidin-4(3H)-one
6-(2-Methoxyphenyl)-3-(((R)-7-((2S,4R)-4-morpholino-2-phenylpiperidine-1-carbonyl)-7-azaspiro[4.5]decan-10-yl)methyl)pyrimidin-4(3H)-one
1-(((R)-7-((2S,4R)-4-((3-Methyloxetan-3-yl)amino)-2-phenylpiperidine-1-carbonyl)-7-azaspiro[4.5]decan-10-yl)methyl)-4-phenylpyridin-2(1H)-one
1-(((R)-7-((2S,4R)-4-((3-(Fluoromethyl)oxetan-3-yl)amino)-2-phenylpiperidine-1-carbonyl)-7-azaspiro[4.5]decan-10-yl)methyl)-4-phenylpyridin-2(1H)-one
1-(((R)-7-((2S,4R)-2-(2,5-Difluorophenyl)-4-(methylamino)piperidine-1-carbonyl)-7-azaspiro[4.5]decan-10-yl)methyl)-4-(2-methoxyphenyl)pyridin-2(1H)-one
3-(((R)-7-((2S,4R)-2-(2,5-Difluorophenyl)-4-(methylamino)piperidine-1-carbonyl)-7-azaspiro[4.5]decan-10-yl)methyl)-6-(o-tolyl)pyrimidin-4(31H)-one
3-(((R)-7-((R)-2-(3-Fluorophenyl)piperazine-1-carbonyl)-7-azaspiro[4.5]decan-10-yl)methyl)-6-(2-methoxyphenyl)pyrimidin-4(3H)-one
3-(((R)-7-((R)-2-(2,5-Difluorophenyl)piperazine-1-carbonyl)-7-azaspiro[4.5]decan-10-yl)methyl)-6-(2-methoxyphenyl)pyrimidin-4(3H)-one
3-(((R)-7-((2S,4R)-2-(2,4-Difluorophenyl)-4-(methylamino)piperidine-1-carbonyl)-7-azaspiro[4.5]decan-10-yl)methyl)-6-(2-methoxyphenyl)pyrimidin-4(3H)-one
1-(((R)-7-((2S,4R)-2-(2-Fluorophenyl)-4-(methylamino)piperidine-1-carbonyl)-7-azaspiro[4.5]decan-10-yl)methyl)-4-(2-methoxyphenyl)pyridin-2(1H)-one
3-(((R)-7-((2S,4R)-2-(3-Fluorophenyl)-4-(methylamino)piperidine-1-carbonyl)-7-azaspiro[4.5]decan-10-yl)methyl)-6-(o-tolyl)pyrimidin-4(3H)-one
1-(((R)-7-((2S,4R)-2-(4-Fluorophenyl)-4-(methylamino)piperidine-1-carbonyl)-7-azaspiro[4.5]decan-10-yl)methyl)-4-(2-methoxyphenyl)pyridin-2(1H)-one
1-(((R)-7-((2S,4R)-2-(3,5-Difluorophenyl)-4-(methylamino)piperidine-1-carbonyl)-7-azaspiro[4.5]decan-10-yl)methyl)-4-phenylpyridin-2(1H)-one
3-(((R)-7-((2S,4R)-2-(3,5-Difluorophenyl)-4-(methylamino)piperidine-1-carbonyl)-7-azaspiro[4.5]decan-10-yl)methyl)-6-phenylpyrimidin-4(3H)-one
4-Chloro-1-(((R)-7-((2S,4R)-2-(2,5-difluorophenyl)-4-(methylamino)piperidine-1-carbonyl)-7-azaspiro[4.5]decan-10-yl)methyl)pyridin-2(1H)-one
3-(((R)-7-((2S,4R)-2-(3,5-Difluorophenyl)-4-(methylamino)piperidine-1-carbonyl)-7-azaspiro[4.5]decan-10-yl)methyl)-6-(2-methoxyphenyl)pyrimidin-4(3H)-one
3-(((R)-7-((2S,4R)-2-(2,5-Difluorophenyl)-4-(methylamino)piperidine-1-carbonyl)-7-azaspiro[4.5]decan-10-yl)methyl)-6-fluoroquinazolin-4(3H)-one
1-(((R)-7-((2S,4R)-2-(3,5-Difluorophenyl)-4-(methylamino)piperidine-1-carbonyl)-7-azaspiro[4.5]decan-10-yl)methyl)-4-(2-methoxyphenyl)pyridin-2(1H)-one
1-(((R)-7-((2S,4R)-2-(2,4-Difluorophenyl)-4-(methylamino)piperidine-1-carbonyl)-7-azaspiro[4.5]decan-10-yl)methyl)-4-(2-methoxyphenyl)-5,6-dihydropyridin-2(1H)-one
1-(((S)-7-((2S,4R)-2-(2,4-difluorophenyl)-4-(methylamino)piperidine-1-carbonyl)-7-azaspiro[4.5]decan-10-yl)methyl)-4-(2-methoxyphenyl)-5,6-dihydropyridin-2(1H)-one
1-(((R)-7-((2S,4R)-2-(2,4-Difluorophenyl)-4-(methylamino)piperidine-1-carbonyl)-7-azaspiro[4.5]decan-10-yl)methyl)pyrimidin-2(1H)-one
2-(((R)-7-((2S,4R)-2-(2,4-Difluorophenyl)-4-(methylamino)piperidine-1-carbonyl)-7-azaspiro[4.5]decan-10-yl)methyl)pyridazin-3(21H)-one
6-(Difluoromethyl)-3-(((R)-7-((2S,4R)-2-(2,4-difluorophenyl)-4-(methylamino)piperidine-1-carbonyl)-7-azaspiro[4.5]decan-10-yl)methyl)pyrimidin-4(3H)-one
1-(((R)-7-((2S,4R)-2-(2,3-Difluorophenyl)-4-(methylamino)piperidine-1-carbonyl)-7-azaspiro[4.5]decan-10-yl)methyl)-4-(2-methoxyphenyl)pyridin-2(1H)-one
3-(((R)-7-((2S,4R)-2-(2,3-Difluorophenyl)-4-(methylamino)piperidine-1-carbonyl)-7-azaspiro[4.5]decan-10-yl)methyl)-6-phenylpyrimidin-4(31H)-one
3-(((R)-7-((2S,4R)-2-(3,4-Difluorophenyl)-4-(methylamino)piperidine-1-carbonyl)-7-azaspiro[4.5]decan-10-yl)methyl)-6-phenylpyrimidin-4(31H)-one
3-(((R)-7-((2S,4R)-4-((2,2-Difluoroethyl)amino)-2-(2,5-difluorophenyl)piperidine-1-carbonyl)-7-azaspiro[4.5]decan-10-yl)methyl)-6-(2-methoxyphenyl)pyrimidin-4(3H)-one
3-(((R)-7-((2S,4R)-4-((3,3-Difluorocyclobutyl)amino)-2-phenylpiperidine-1-carbonyl)-7-azaspiro[4.5]decan-10-yl)methyl)-6-phenylpyrimidin-4(31H)-one
3-(((R)-7-((2S,4R)-2-(2,3-Difluorophenyl)-4-(methylamino)piperidine-1-carbonyl)-7-azaspiro[4.5]decan-10-yl)methyl)-6-(o-tolyl)pyrimidin-4(3H)-one
3-(((R)-7-((2S,4R)-2-(2,4-Difluorophenyl)-4-(methylamino)piperidine-1-carbonyl)-7-azaspiro[4.5]decan-10-yl)methyl)-6-(o-tolyl)pyrimidin-4(3H)-one
3-(((R)-7-((2S,4R)-2-(3,4-Difluorophenyl)-4-(methylamino)piperidine-1-carbonyl)-7-azaspiro[4.5]decan-10-yl)methyl)-6-(2-methoxyphenyl)pyrimidin-4(3H)-one
3-(((R)-7-((2S,4R)-2-(3,4-Difluorophenyl)-4-(methylamino)piperidine-1-carbonyl)-7-azaspiro[4.5]decan-10-yl)methyl)-6-(o-tolyl)pyrimidin-4(3H)-one
3-(((R)-7-((2S,4R)-2-(2,3-Difluorophenyl)-4-(methylamino)piperidine-1-carbonyl)-7-azaspiro[4.5]decan-10-yl)methyl)-6-(2-fluorophenyl)pyrimidin-4(3H)-one
3-(((R)-7-((2S,4R)-2-(2,4-Difluorophenyl)-4-(methylamino)piperidine-1-carbonyl)-7-azaspiro[4.5]decan-10-yl)methyl)-6-(2-fluorophenyl)pyrimidin-4(3H)-one
3-(((R)-7-((2S,4R)-2-(3,4-Difluorophenyl)-4-(methylamino)piperidine-1-carbonyl)-7-azaspiro[4.5]decan-10-yl)methyl)-6-(2-fluorophenyl)pyrimidin-4(3H)-one
3-(((R)-7-((2S,4R)-2-(3,5-Difluorophenyl)-4-(methylamino)piperidine-1-carbonyl)-7-azaspiro[4.5]decan-10-yl)methyl)-6-(2-fluorophenyl)pyrimidin-4(3H)-one
6-Phenyl-3-(((R)-7-((2S,4R)-2-phenyl-4-((pyridin-2-ylmethyl)amino)piperidine-1-carbonyl)-7-azaspiro[4.5]decan-10-yl)methyl)pyrimidin-4(31H)-one
3-(((R)-7-((2S,4R)-2-(2,5-Difluorophenyl)-4-(methylamino)piperidine-1-carbonyl)-7-azaspiro[4.5]decan-10-yl)methyl)-6-(2-fluorophenyl)pyrimidin-4(3H)-one
3-(((R)-7-((2S,4R)-2-(3,5-Difluorophenyl)-4-(methylamino)piperidine-1-carbonyl)-7-azaspiro[4.5]decan-10-yl)methyl)-6-(o-tolyl)pyrimidin-4(31H)-one
3-(((R)-7-((2S,4R)-4-((1-Methyl-1H-pyrazol-3-yl)amino)-2-phenylpiperidine-1-carbonyl)-7-azaspiro[4.5]decan-10-yl)methyl)-6-phenylpyrimidin-4(3H)-one
3-(((R)-7-((2S,4R)-4-(((5-Fluoropyridin-2-yl)methyl)amino)-2-phenylpiperidine-1-carbonyl)-7-azaspiro[4.5]decan-10-yl)methyl)-6-phenylpyrimidin-4(31H)-one
3-(((R)-7-((2S,4R)-2-(2,3-Difluorophenyl)-4-(methylamino)piperidine-1-carbonyl)-7-azaspiro[4.5]decan-10-yl)methyl)-6-(2-methoxyphenyl)pyrimidin-4(3H)-one
6-Phenyl-3-(((R)-7-((2S,4R)-2-phenyl-4-(phenylamino)piperidine-1-carbonyl)-7-azaspiro[4.5]decan-10-yl)methyl)pyrimidin-4(3H)-one
3-(((R)-7-((2S,4R)-4-((1-Methylcyclopropyl)amino)-2-phenylpiperidine-1-carbonyl)-7-azaspiro[4.5]decan-10-yl)methyl)-6-phenylpyrimidin-4(3H)-one
3-(((R)-7-((2S,4R)-4-((2,2-Difluoroethyl)amino)-2-(2,5-difluorophenyl)piperidine-1-carbonyl)-7-azaspiro[4.5]decan-10-yl)methyl)-6-(difluoromethyl)pyrimidin-4(3H)-one
3-(((R)-7-((2S,4R)-2-(3,5-Difluorophenyl)-4-(methylamino)piperidine-1-carbonyl)-7-azaspiro[4.5]decan-10-yl)methyl)-6-fluoroquinazolin-4(3H)-one
tert-Butyl ((2S,4R)-2-(2,5-difluorophenyl)-1-((R)-10-((6-oxo-4-phenylpyrimidin-1 (6H)-yl)methyl)-7-azaspiro[4.5]decane-7-carbonyl)piperidin-4-yl)glycinate
N-((2S,4R)-2-(2,5-Difluorophenyl)-1-((R)-10-((6-oxo-4-phenylpyrimidin-1 (6H)-yl)methyl)-7-azaspiro[4.5]decane-7-carbonyl)piperidin-4-yl)acetamide
3-(((R)-7-((2S,4R)-2-(2,5-Difluorophenyl)-4-(ethylamino)piperidine-1-carbonyl)-7-azaspiro[4.5]decan-10-yl)methyl)-6-phenylpyrimidin-4(31H)-one
3-(((R)-7-((2S,4R)-4-((2,2-Difluoroethyl)amino)-2-(2,5-difluorophenyl)piperidine-1-carbonyl)-7-azaspiro[4.5]decan-10-yl)methyl)-6-fluoroquinazolin-4(31H)-one
((2S,4R)-2-(2,5-Difluorophenyl)-1-((R)-10-((6-oxo-4-phenylpyrimidin-1(6H)-yl)methyl)-7-azaspiro[4.5]decane-7-carbonyl)piperidin-4-yl)glycine
4-(((R)-7-((2S,4R)-2-(2,5-Difluorophenyl)-4-(methylamino)piperidine-1-carbonyl)-7-azaspiro[4.5]decan-10-yl)methyl)morpholin-3-one
3-(((R)-7-((2S,4R)-2-(2,5-Difluorophenyl)-4-(isopropylamino)piperidine-1-carbonyl)-7-azaspiro[4.5]decan-10-yl)methyl)-6-phenylpyrimidin-4(3H)-one
3-(((R)-7-((2S,4R)-4-((2-Hydroxyethyl)amino)-2-phenylpiperidine-1-carbonyl)-7-azaspiro[4.5]decan-10-yl)methyl)-6-phenylpyrimidin-4(31H)-one
3-(((R)-7-((2S,4R)-2-(2,5-Difluorophenyl)-4-(ethylamino)piperidine-1-carbonyl)-7-azaspiro[4.5]decan-10-yl)methyl)-6-fluoroquinazolin-4(3H)-one
6-Cyclopropyl-3-(((R)-7-((2S,4R)-2-(2,5-difluorophenyl)-4-(methylamino)piperidine-1-carbonyl)-7-azaspiro[4.5]decan-10-yl)methyl)pyrimidin-4(3H)-one
N-((2S,4R)-2-(2,5-Difluorophenyl)-1-((R)-10-((6-oxo-4-phenylpyrimidin-1 (6H)-yl)methyl)-7-azaspiro[4.5]decane-7-carbonyl)piperidin-4-yl)cyclopropanecarboxamide
N-((2S,4R)-2-(2,5-Difluorophenyl)-1-((R)-10-((6-oxo-4-phenylpyrimidin-1 (6H)-yl)methyl)-7-azaspiro[4.5]decane-7-carbonyl)piperidin-4-yl)picolinamide
3-(((R)-7-((2S,4R)-2-(2,3-Difluorophenyl)-4-(methylamino)piperidine-1-carbonyl)-7-azaspiro[4.5]decan-10-yl)methyl)-6-fluoroquinazolin-4(3H)-one
6-Fluoro-3-(((R)-7-((2S,4R)-2-(3-fluorophenyl)-4-(methylamino)piperidine-1-carbonyl)-7-azaspiro[4.5]decan-10-yl)methyl)quinazolin-4(3H)-one
1-(((R)-7-((2S,4R)-2-(2,5-Difluorophenyl)-4-(methylamino)piperidine-1-carbonyl)-7-azaspiro[4.5]decan-10-yl)methyl)-4-phenyl-5,6-dihydropyridin-2(1H)-one
3-(((R)-7-((2S,4R)-2-(2,5-Difluorophenyl)-4-((pyrimidin-2-ylmethyl)amino)piperidine-1-carbonyl)-7-azaspiro[4.5]decan-10-yl)methyl)-6-phenylpyrimidin-4(3H)-one
3-(((R)-7-((2S,4R)-2-(2,5-Difluorophenyl)-4-((pyrimidin-4-ylmethyl)amino)piperidine-1-carbonyl)-7-azaspiro[4.5]decan-10-yl)methyl)-6-phenylpyrimidin-4(3H)-one
6-Fluoro-3-(((R)-7-((2S,4R)-4-morpholino-2-phenylpiperidine-1-carbonyl)-7-azaspiro[4.5]decan-10-yl)methyl)quinazolin-4(3H)-one hydrochloride
N-((2S,4R)-2-(2,5-Difluorophenyl)-1-((R)-10-((6-fluoro-4-oxoquinazolin-3(4H)-yl)methyl)-7-azaspiro[4.5]decane-7-carbonyl)piperidin-4-yl)acetamide
N-((2S,4R)-2-(2,5-Difluorophenyl)-1-((R)-10-((6-fluoro-4-oxoquinazolin-3(4H)-yl)methyl)-7-azaspiro[4.5]decane-7-carbonyl)piperidin-4-yl)cyclopropanecarboxamide
3-(((R)-7-((2S,4R)-2-(2,5-Difluorophenyl)-4-(dimethylamino)piperidine-1-carbonyl)-7-azaspiro[4.5]decan-10-yl)methyl)-6-fluoroquinazolin-4(3H)-one
(2S,4R)-1-((R)-10-((6-Oxo-4-phenylpyrimidin-1(6H)-yl)methyl)-7-azaspiro[4.5]decane-7-carbonyl)-2-phenylpiperidine-4-carboxylic acid
(2S,4R)—N-Methyl-1-((R)-10-((6-oxo-4-phenylpyrimidin-1 (6H)-yl)methyl)-7-azaspiro[4.5]decane-7-carbonyl)-2-phenylpiperidine-4-carboxamide
(2S,4R)—N,N-Dimethyl-1-((R)-10-((6-oxo-4-phenylpyrimidin-1(6H)-yl)methyl)-7-azaspiro[4.5]decane-7-carbonyl)-2-phenylpiperidine-4-carboxamide
1-(((R)-7-((2S,4R)-2-(2,5-Difluorophenyl)-4-(methylamino)piperidine-1-carbonyl)-7-azaspiro[4.5]decan-10-yl)methyl)-4-phenyl-1,5-dihydro-2H-pyrrol-2-one
4-Chloro-1-(((R)-7-((2S,4R)-2-(2,5-difluorophenyl)-4-(ethylamino)piperidine-1-carbonyl)-7-azaspiro[4.5]decan-10-yl)methyl)pyridin-2(1H)-one
1-(((R)-7-((2S,4R)-2-(2,5-Difluorophenyl)-4-(ethylamino)piperidine-1-carbonyl)-7-azaspiro[4.5]decan-10-yl)methyl)-4-phenylpyridin-2(1H)-one
3-(((R)-7-((2S,4R)-4-Amino-2-(3,5-difluorophenyl)piperidine-1-carbonyl)-7-azaspiro[4.5]decan-10-yl)methyl)-6-fluoroquinazolin-4(3H)-one hydrochloride
6-Cyclopropyl-3-(((R)-7-((2S,4R)-4-((2,2-difluoroethyl)amino)-2-(2,5-difluorophenyl)piperidine-1-carbonyl)-7-azaspiro[4.5]decan-10-yl)methyl)pyrimidin-4(3H-one
3-(((R)-7-((2S,4R)-4-((3,3-Difluorocyclobutyl)amino)-2-(2,5-difluorophenyl)piperidine-1-carbonyl)-7-azaspiro[4.5]decan-10-yl)methyl)-6-(difluoromethyl)pyrimidin-4(3H)-one
3-(((R)-7-((2S,4R)-2-(3,5-Difluorophenyl)-4-(dimethylamino)piperidine-1-carbonyl)-7-azaspiro[4.5]decan-10-yl)methyl)-6-fluoroquinazolin-4(3H)-one hydrochloride
4-Chloro-1-(((R)-7-((2S,4R)-4-((2,2-difluoroethyl)amino)-2-(2,5-difluorophenyl)piperidine-1-carbonyl)-7-azaspiro[4.5]decan-10-yl)methyl)pyridin-2(1H)-one
4-Chloro-1-(((R)-7-((2S,4R)-4-((3,3-difluorocyclobutyl)amino)-2-(2,5-difluorophenyl)piperidine-1-carbonyl)-7-azaspiro[4.5]decan-10-yl)methyl)pyridin-2(1H)-one
3-(((S)-7-((R)-2-(2,5-Difluorophenyl)piperazine-1-carbonyl)-10-methoxy-7-azaspiro[4.5]decan-10-yl)methyl)-6-(2-methoxyphenyl)pyrimidin-4(3H)-one
3-(((R)-7-((2S,5R)-5-Amino-2-phenylpiperidine-1-carbonyl)-7-azaspiro[4.5]decan-10-yl)methyl)-6-phenylpyrimidin-4(3H)-one
3-(((R)-7-((2R,5S)-5-amino-2-phenylpiperidine-1-carbonyl)-7-azaspiro[4.5]decan-10-yl)methyl)-6-phenylpyrimidin-4(3H)-one
3-(((R)-7-((2S,4R)-2-(2,5-Difluorophenyl)-4-(isopropylamino)piperidine-1-carbonyl)-7-azaspiro[4.5]decan-10-yl)methyl)-6-fluoroquinazolin-4(3H)-one
3-(((S)-7-((2S,4R)-2-(2,5-Difluorophenyl)-4-(isopropylamino)piperidine-1-carbonyl)-10-methoxy-7-azaspiro[4.5]decan-10-yl)methyl)-6-(2-methoxyphenyl)pyrimidin-4(3H)-one
3-(((R)-7-((2S,4R)-2-(2,4-Difluorophenyl)-4-(methylamino)piperidine-1-carbonyl)-7-azaspiro[4.5]decan-10-yl)methyl)-6-fluoroquinazolin-4(3H)-one
3-(((R)-7-((2S,4R)-2-(3,5-Difluorophenyl)-4-(isopropylamino)piperidine-1-carbonyl)-7-azaspiro[4.5]decan-10-yl)methyl)-6-phenylpyrimidin-4(31H)-one
3-(((R)-7-((2S,4R)-4-(((3-Methylpyridin-2-yl)methyl)amino)-2-phenylpiperidine-1-carbonyl)-7-azaspiro[4.5]decan-10-yl)methyl)-6-phenylpyrimidin-4(3H)-one
3-(((R)-7-((2S,4R)-4-(((4,6-Dimethylpyrimidin-2-yl)methyl)amino)-2-phenylpiperidine-1-carbonyl)-7-azaspiro[4.5]decan-10-yl)methyl)-6-phenylpyrimidin-4(3H)-one
3-(((R)-7-((2S,4R)-2-(3-Fluorophenyl)-4-((2-methoxyethyl)amino)piperidine-1-carbonyl)-7-azaspiro[4.5]decan-10-yl)methyl)-6-phenylpyrimidin-4(31H)-one
3-(((R)-7-((2S,4R)-4-((2-Fluoroethyl)amino)-2-(3-fluorophenyl)piperidine-1-carbonyl)-7-azaspiro[4.5]decan-10-yl)methyl)-6-phenylpyrimidin-4(31H)-one
3-((2S,4R)-2-(3-Fluorophenyl)-1-((R)-10-((6-oxo-4-phenylpyrimidin-1(6H)-yl)methyl)-7-azaspiro[4.5]decane-7-carbonyl)piperidin-4-yl)oxazolidin-2-one
6-Chloro-3-(((R)-7-((2S,4R)-2-(2,5-difluorophenyl)-4-(methylamino)piperidine-1-carbonyl)-7-azaspiro[4.5]decan-10-yl)methyl)quinazolin-4(31H)-one
3-(((R)-7-((2S,4R)-2-(2,5-Difluorophenyl)-4-(methylamino)piperidine-1-carbonyl)-7-azaspiro[4.5]decan-10-yl)methyl)quinazolin-4(31H)-one
3-(((R)-7-((2S,5R)-5-(2,5-Difluorophenyl)-2-methylmorpholine-4-carbonyl)-7-azaspiro[4.5]decan-10-yl)methyl)-6-fluoroquinazolin-4(3H)-one
3-(((R)-7-((2S,5R)-5-(2,5-Difluorophenyl)-2-methylmorpholine-4-carbonyl)-7-azaspiro[4.5]decan-10-yl)methyl)-6-phenylpyrimidin-4(31H)-one
   or a stereoisomer, tautomer, hydrate, N-oxide derivative or pharmaceutically acceptable salt thereof.

In certain embodiments there is provided a compound, stereoisomer, tautomer, hydrate, N-oxide derivative or pharmaceutically acceptable salt as described above that is an inhibitor of USP19, preferably human USP19.

USP19 inhibitor compounds are also disclosed in WO2018/020242, WO2020/115500, WO2019/150119, and WO2020/115501, each of which is expressly incorporated herein by reference. Analogues of the compounds disclosed in WO2018/020242, WO2020/115500, WO2019/150119, and WO2020/115501 according to formula (I)—i.e. where R0 is H, F, NH2, or OCH3—are expressly incorporated herein and can be obtained by the skilled person following the synthesis protocols provided herein and in WO2018/020242, WO2020/115500, WO2019/150119, and WO2020/115501.

In a second aspect the present invention provides a pharmaceutical composition comprising a compound according to any embodiment of the first aspect, or a stereoisomer, tautomer, hydrate, N-oxide derivative or pharmaceutically acceptable salt thereof, and at least one pharmaceutically acceptable excipient.

Pharmaceutical compositions may be formulated according to their particular use and purpose by mixing, for example, excipient, binding agent, lubricant, disintegrating agent, coating material, emulsifier, suspending agent, solvent, stabilizer, absorption enhancer and/or ointment base. The composition may be suitable for oral, injectable, rectal or topical administration.

Suitable pharmaceutically acceptable excipients would be known by the person skilled in the art, for example: fats, water, physiological saline, alcohol (e.g. ethanol), glycerol, polyols, aqueous glucose solution, extending agent, disintegrating agent, binder, lubricant, wetting agent, stabilizer, emulsifier, dispersant, preservative, sweetener, colorant, seasoning agent or aromatizer, concentrating agent, diluent, buffer substance, solvent or solubilizing agent, chemical for achieving storage effect, salt for modifying osmotic pressure, coating agent or antioxidant, saccharides such as lactose or glucose; starch of corn, wheat or rice; fatty acids such as stearic acid; inorganic salts such as magnesium metasilicate aluminate or anhydrous calcium phosphate; synthetic polymers such as polyvinylpyrrolidone or polyalkylene glycol; alcohols such as stearyl alcohol or benzyl alcohol; synthetic cellulose derivatives such as methylcellulose, carboxymethylcellulose, ethylcellulose or hydroxypropylmethylcellulose; and other conventionally used additives such as gelatin, talc, plant oil and gum arabic.

For example, the pharmaceutical composition may be administered orally, such as in the form of tablets, coated tablets, hard or soft gelatine capsules, solutions, emulsions, or suspensions. Administration can also be carried out rectally, for example using suppositories, locally or percutaneously, for example using ointments, creams, gels or solution, or parenterally, for example using injectable solutions.

For the preparation of tablets, coated tablets or hard gelatine capsules, the compounds of the present invention may be admixed with pharmaceutically inert, inorganic or organic excipients. Examples of suitable excipients include lactose, mize starch or derivatives thereof, talc or stearic acid or salts thereof. Suitable excipients for use with soft gelatine capsules include, for example, vegetable oils, waxes, fats and semi-solid or liquid polyols.

For the preparation of solutions and syrups, excipients include, for example, water, polyols, saccharose, invert sugar and glucose.

For injectable solutions, excipients include, for example, water, alcohols, polyols, glycerine and vegetable oil.

For suppositories and for local and percutaneous application, excipients include, for example, natural or hardened oils, waxes, fats and semi-solid or liquid polyols.

The pharmaceutical compositions may also contain preserving agents, solubalizing agents, stabilizing agents, wetting agents, emulsifiers, sweeteners, colorants, odorants, buffers, coating agents and/or antioxidants.

For combination therapies, the second drug may be provided in pharmaceutical composition with the present invention or may be provided separately.

Thus, a pharmaceutical formulation for oral administration may, for example, be granule, tablet, sugar-coated tablet, capsule, pill, suspension or emulsion. For parenteral injection for, for example, intravenous, intramuscular or subcutaneous use, a sterile aqueous solution may be provided that may contain other substances including, for example, salts and/or glucose to make to solution isotonic. The anti-cancer agent may also be administered in the form of a suppository or pessary, or may be applied topically in the form of a lotion, solution, cream, ointment or dusting powder.

In a further aspect the invention provides a compound according to the first aspect, including a stereoisomer, tautomer, hydrate, N-oxide derivative or pharmaceutically acceptable salt thereof, for use in therapy.

In a further aspect the invention provides a pharmaceutical composition according to the second aspect for use in therapy.

In a further aspect the invention provides a compound according to any embodiment of the first aspect, or a stereoisomer, tautomer, hydrate, N-oxide derivative or pharmaceutically acceptable salt thereof, for use in the treatment and/or prevention of cancer.

In a further aspect the invention provides a pharmaceutical composition according to the second aspect for use in the treatment and/or prevention of cancer.

In a further aspect the invention provides a method of treating or preventing cancer comprising administering to a subject a compound, including a stereoisomer, tautomer, hydrate, N-oxide derivative or pharmaceutically acceptable salt thereof, according to any embodiment of the first aspect of the invention or a pharmaceutical composition according to any embodiment of the second aspect of the invention.

In a further aspect the invention provides a use of a compound, including a stereoisomer, tautomer, hydrate, N-oxide derivative or pharmaceutically acceptable salt thereof, according to any embodiment of the first aspect in the manufacture of a medicament for treating or preventing cancer.

Cancers or neoplastic conditions suitable to be treated with the compounds or compositions according to the invention include, for example: prostate cancer, colon cancer, breast cancer, lung cancer, kidney cancer, CNS cancers (e.g. neuroblastomas, glioblastomas), osteosarcoma, haematological malignancies (e.g. leukemia, multiple myeloma and mantle cell lymphoma). In certain preferred embodiments the cancer is associated with p53 dysregulation. In certain preferred embodiments, the cancer is selected from a haematological malignancy (e.g. mantle cell lymphoma, multiple myeloma), prostate cancer, a neuroblastoma, or a glioblastoma. In certain preferred embodiments, the cancer is neuroblastoma or breast cancer.

It is demonstrated herein that the potent USP19 inhibitory compounds effectively reduce fat accumulation in vivo. Gene knockout studies have described a possible association between USP19 and fat accumulation (Coyne et al, Diabetologia, 2019, 62, 136-146, incorporated herein by reference). However, the effects seen in these studies need to be considered alongside the possible confounding factors inherent in knockout studies such as altered developmental or underlying physiological processes. For these reasons, acute or chronic pharmacological inhibition of an enzyme does not always result in similar physiological outcomes to genetic ablation.

The data provided herein demonstrates that pharmacological inhibition of USP19 can reduce fat accumulation in a wild-type background. Taken together, the in vitro and in vivo data demonstrate that compounds which potently inhibit USP19 activity can effectively treat obesity.

In a further aspect is provided a compound according to the first aspect, or a pharmaceutically acceptable salt, tautomer, stereoisomer or N-oxide derivative thereof, for use in a method of treating obesity.

In a further aspect is provided a pharmaceutical composition according to the second aspect for use in a method of treating obesity.

Also provided in accordance with the invention is a method of treating obesity comprising administering to a subject in need thereof an effective amount of a compound, pharmaceutically acceptable salt, tautomer, stereoisomer or N-oxide derivative according to the first aspect, or an effective amount of a pharmaceutical composition according to the second aspect.

It is further demonstrated herein that the potent USP19 inhibitory compounds provided herein can effectively treat insulin resistance. Gene knockout studies have described an association between USP19 and insulin sensitivity (Coyne et al, supra). Coyne et al. describe an improvement in insulin sensitivity in USP19 knockout mice but, as noted above, it could not be assumed that the effects would translate to pharmacological inhibition of USP19 in wild-type subjects.

The data provided herein demonstrates that pharmacological inhibition of USP19 can effectively treat insulin resistance (e.g. type II diabetes).

In a further aspect is provided a compound as defined in relation to the first aspect of the invention, or a pharmaceutically acceptable salt, tautomer, stereoisomer or N-oxide derivative thereof, for use in a method of treating insulin resistance.

In a further aspect of the invention is provided a compound as defined in relation to the first aspect of the invention, or a pharmaceutically acceptable salt, tautomer, stereoisomer or N-oxide derivative thereof, for use in a method of treating type II diabetes.

In a further aspect of the invention is provided a pharmaceutical composition according to the second aspect for use in a method of treating insulin resistance.

In a further aspect of the invention is provided a pharmaceutical composition according to the second aspect for use in a method of treating type II diabetes.

Also provided in accordance with the invention is a method of treating insulin resistance comprising administering to a subject in need thereof an effective amount of a compound, pharmaceutically acceptable salt, tautomer, stereoisomer or N-oxide derivative as defined in relation to the first aspect of the invention, or an effective amount of a pharmaceutical composition comprising a compound, pharmaceutically acceptable salt, tautomer, stereoisomer or N-oxide derivative as defined in relation to the first aspect of the invention.

Also provided in accordance with the invention is a method of treating type II diabetes comprising administering to a subject in need thereof an effective amount of a compound, pharmaceutically acceptable salt, tautomer, stereoisomer or N-oxide derivative as defined in relation to the first aspect of the invention, or an effective amount of a pharmaceutical composition comprising a compound, pharmaceutically acceptable salt, tautomer, stereoisomer or N-oxide derivative as defined in relation to the first aspect of the invention.

It is demonstrated in the accompanying Examples that compounds provided herein are potent USP19 inhibitors and further that potent USP19 inhibitory compounds effectively treat muscle loss in an in vivo disease model. Taken together, the in vitro and in vivo data demonstrate that compounds which potently inhibit USP19 activity can effectively treat muscular atrophy.

In a further aspect is provided a compound as defined in relation to the first aspect of the invention, or a pharmaceutically acceptable salt, tautomer, stereoisomer or N-oxide derivative thereof, for use in a method of treating muscular atrophy.

In a further aspect the invention provides a compound as defined in relation to the first aspect, or a pharmaceutically acceptable salt, tautomer, stereoisomer or N-oxide derivative thereof, for use in a method of treating cachexia or sarcopenia.

In a further aspect of the invention is provided a pharmaceutical composition according to the second aspect for use in a method of treating muscular atrophy.

In a further aspect of the invention is provided a pharmaceutical composition according to the second aspect for use in a method of treating cachexia or sarcopenia.

Also provided in accordance with the invention is a method of treating muscular atrophy comprising administering to a subject in need thereof an effective amount of a compound, pharmaceutically acceptable salt, tautomer, stereoisomer or N-oxide derivative as defined in relation to the first aspect of the invention, or an effective amount of a pharmaceutical composition comprising a compound, pharmaceutically acceptable salt, tautomer, stereoisomer or N-oxide derivative as defined in relation to the first aspect of the invention.

Also provided in accordance with the invention is a method of treating cachexia or sarcopenia comprising administering to a subject in need thereof an effective amount of a compound, pharmaceutically acceptable salt, tautomer, stereoisomer or N-oxide derivative as defined in relation to the first aspect of the invention, or an effective amount of a pharmaceutical composition comprising a compound, pharmaceutically acceptable salt, tautomer, stereoisomer or N-oxide derivative as defined in relation to the first aspect of the invention.

Muscle atrophy, cachexia or sarcopenia may be associated with or induced by HIV infection/AIDS, heart failure, rheumatoid arthritis, chronic obstructive pulmonary disease (COPD), cystic fibrosis, multiple sclerosis, motor neuron disease (MND), Parkinson's disease, dementia, or cancer.

In a further aspect, the invention provides a compound or composition according to any embodiment of the first aspect or second aspect for use in the treatment and/or prevention of Parkinson's Disease. In a further aspect, the invention provides a method of treating or preventing Parkinson's Disease comprising administering an effective amount of a compound, pharmaceutically acceptable salt, tautomer, stereoisomer or N-oxide derivative thereof, or pharmaceutical composition according to the invention to a subject. In a further aspect, the invention provides the use of a compound according to the invention, a or pharmaceutically acceptable salt, tautomer, stereoisomer or N-oxide derivative thereof, in the manufacture of a medicament for the treatment of Parkinson's Disease.

The compound or composition of the invention may be used in monotherapy and/or a combination modality. Suitable agents to be used in such combination modalities with compounds or compositions according to the invention include one or more of anti-cancer agents, anti-inflammatory agents, immuno-modulatory agents, for example immuno-suppressive agents, neurological agents, anti-diabetic agents, anti-viral agents, anti-bacterial agents and/or radiation therapy.

Agents used in combination with the compounds of the present invention may target the same or a similar biological pathway to that targeted by the compounds of the present invention or may act on a different or unrelated pathway.

Depending on the disease to be treated, a variety of combination partners may be coadministered with the compounds of the present invention. The second active ingredient may include, but is not restricted to: alkylating agents, including cyclophosphamide, ifosfamide, thiotepa, melphalan, chloroethylnitrosourea and bendamustine; platinum derivatives, including cisplatin, oxaliplatin, carboplatin and satraplatin; antimitotic agents, including vinca alkaloids (vincristine, vinorelbine and vinblastine), taxanes (paclitaxel, docetaxel), epothilones and inhibitors of mitotic kinases including aurora and polo kinases; topoisomerase inhibitors, including anthracyclines, epipodophyllotoxins, camptothecin and analogues of camptothecin; antimetabolites, including 5-fluorouracil, capecitabine, cytarabine, gemcitabine, 6-mercaptopurine, 6-thioguanine, fludarabine, methotrexate and premetrexed; protein kinase inhibitors, including imatinib, gefitinib, sorafenib, sunitinib, erlotinib, dasatinib, and lapatinib; proteosome inhibitors, including bortezomib; histone deacetylase inhibitors, including valproate and SAHA; antiangiogenic drugs, including bevacizumab; monoclonal antibodies, including trastuzumab, rituximab, alemtuzumab, tositumomab, cetuximab, panitumumab; conjugates of myoclonal antibodies, including Gemtuzumab ozogamicin, Ibritumomab tiuxetan; hormonal therapies, including antiestrogens (tamoxifen, raloxifen, anastrazole, letrozole, examestane) antiandrogens (Flutamide, Biclutamide) and Luteinisng Hormone Analogues or antagonists.

In regard to aspects of the invention relating to therapeutic use of compounds according to the invention, the compounds may be administered to the subject in need of treatment in an “effective amount”. The term “effective amount” refers to the amount or dose of a compound which, upon single or multiple dose administration to a subject, provides therapeutic efficacy in the treatment of disease. Therapeutically effective amounts of a compound according to the invention can comprise an amount in the range of from about 0.1 mg/kg to about 20 mg/kg per single dose. A therapeutic effective amount for any individual patient can be determined by the healthcare professional by methods understood by the skilled person. The amount of compound administered at any given time point may be varied so that optimal amounts of the compound, whether employed alone or in combination with any other therapeutic agent, are administered during the course of treatment. It is also contemplated to administer compounds according to the invention, or pharmaceutical compositions comprising such compounds, in combination with any other cancer treatment, as a combination therapy.

For combination therapies, the second drug may be provided in pharmaceutical composition with the present invention or may be provided separately.

### Routes of Administration

In certain preferred embodiments, treatment according to the invention comprises administering the therapeutic agent (that is, the compound, pharmaceutically acceptable salt, tautomer, stereoisomer or N-oxide derivative, or pharmaceutical composition for use according to the invention) parenterally.

In certain preferred embodiments, the therapeutic agent is administered orally.

In certain preferred embodiments the therapeutic agent is administered intravenously. In certain preferred embodiments, the therapeutic agent is administered intraperitoneally. In certain preferred embodiments, the therapeutic agent is administered subcutaneously.

### Dosage Regimen

In certain preferred embodiments of the invention, treatment comprises administering the therapeutic agent (that is, the compound, pharmaceutically acceptable salt, tautomer, stereoisomer or N-oxide derivative, or pharmaceutical composition for use according to the invention) at a dose in the range of from 10 to 150 mg/kg. In such embodiments, the dose refers to the amount of the active ingredient administered to the subject per single administration.

In certain preferred embodiments, treatment comprises administering the therapeutic agent at a dose in the range of from 25 to 125 mg/kg. In certain preferred embodiments, treatment comprises administering the therapeutic agent at a dose in the range of from 50 to 100 mg/kg.

In certain preferred embodiments, the method comprises administering the therapeutic agent at a dose of 75 mg/kg.

In certain preferred embodiments, treatment comprises administering the therapeutic agent (that is, the compound, pharmaceutically acceptable salt, tautomer, stereoisomer or N-oxide derivative, or pharmaceutical composition for use according to the invention) 1, 2, 3 or 4 times daily. In certain preferred embodiments, the therapeutic agent is administered once or twice daily, most preferably twice daily.

In certain preferred embodiments, the therapeutic agent is administered at a daily dosage in the range of from 10 to 300 mg/kg. That is, the total amount of active agent administered to the subject in one day is in the range of from 10-300 mg/kg. In such embodiments, the therapeutic agent may be administered once or multiple times per day as described herein, provided the total daily dosage is in the indicated range.

In certain preferred embodiments, the therapeutic agent is administered at a daily dosage in the range of from 50 to 250 mg/kg. In certain preferred embodiments, the therapeutic agent is administered at a daily dosage in the range of from 75 to 250 mg/kg. In certain preferred embodiments, the therapeutic agent is administered at a daily dosage in the range of from 100 to 200 mg/kg. In certain preferred embodiments, the therapeutic agent is administered at a daily dosage of 150 mg/kg.

In certain preferred embodiments, the therapeutic agent (for example a compound as provided herein) is administered at a dose of 75 mg/kg twice daily.

In regard to aspects of the invention relating to therapeutic use of compounds according to the invention, in preferred embodiments the subject to be treated is human.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

### EXAMPLES

The present invention will now be described in relation to several examples.

The examples indicated below were synthesised according to the methods described subsequently. IC₅₀ values were determined as described below and are represented in the following table.

| Example | USP19 IC₅₀ |
|---|---|
| Number | activity |
| 1 | ** |
| 2 | *** |
| 3 | ** |
| 4 | * |
| 5 | * |
| 6 | ** |
| 7 | * |
| 8 | ** |
| 9 | *** |
| 10 | ** |
| 11 | **** |
| 12 | ** |
| 13 | ** |
| 14 | **** |
| 15 | * |
| 16 | **** |
| 17 | *** |
| 18 | *** |
| 19 | **** |
| 20 | *** |
| 21 | **** |
| 22 | *** |
| 23 | **** |
| 24 | * |
| 25 | **** |
| 26 | ** |
| 27 | **** |
| 28 | *** |
| 29 | **** |
| 30 | ** |
| 31 | *** |
| 32 | *** |
| 33 | *** |
| 34 | *** |
| 35 | **** |
| 36 | **** |
| 37 | **** |
| 38 | **** |
| 39 | *** |
| 40 | **** |
| 41 | *** |
| 42 | *** |
| 43 | *** |
| 44 | *** |
| 45 | **** |
| 46 | *** |
| 47 | **** |
| 48 | **** |
| 49 | ** |
| 50 | *** |
| 51 | ** |
| 52 | ** |
| 53 | **** |
| 54 | **** |
| 55 | **** |
| 56 | **** |
| 57 | **** |
| 58 | **** |
| 59 | **** |
| 60 | **** |
| 61 | **** |
| 62 | **** |
| 63 | **** |
| 64 | **** |
| 65 | **** |
| 66 | **** |
| 67 | **** |
| 68 | *** |
| 69 | **** |
| 70 | **** |
| 71 | **** |
| 72 | **** |
| 73 | **** |
| 74 | **** |
| 75 | *** |
| 76 | * |
| 77 | **** |
| 78 | **** |
| 79 | **** |
| 80 | **** |
| 81 | **** |
| 82 | **** |
| 83 | **** |
| 84 | **** |
| 85 | **** |
| 86 | **** |
| 87 | **** |
| 88 | **** |
| 89 | **** |
| 90 | **** |
| 91 | **** |
| 92 | **** |
| 93 | *** |
| 94 | **** |
| 95 | **** |
| 96 | *** |
| 97 | **** |
| 98 | **** |
| 99 | **** |
| 100 | **** |
| 101 | **** |
| 102 | **** |
| 103 | **** |
| 104 | **** |
| 105 | **** |
| 106 | **** |
| 107 | **** |
| 108 | **** |
| 109 | **** |
| 110 | *** |
| 111 | **** |
| 112 | *** |
| 113 | **** |
| 114 | **** |
| 115 | **** |
| 116 | **** |
| 117 | **** |
| 118 | **** |
| 119 | **** |
| 120 | **** |
| 121 | **** |
| 122 | **** |
| 123 | **** |
| 124 | **** |
| 125 | **** |
| 126 | **** |
| 127 | **** |
| 128 | ** |
| 129 | *** |
| 130 | **** |
| 131 | **** |
| 132 | **** |
| 133 | **** |
| 134 | **** |
| 135 | **** |
| 136 | **** |
| 137 | **** |
| 138 | **** |
| 139 | **** |
| 140 | **** |
| 141 | **** |
| 142 | **** |
| 143 | **** |
| 144 | **** |
| 145 | **** |
| 146 | **** |
| 147 | ** |
| 148 | **** |
| 149 | **** |
| 150 | * |
| 151 | **** |
| 152 | *** |
| 153 | **** |
| 154 | **** |
| 155 | **** |
| 156 | **** |
| 157 | **** |
| 158 | **** |
| 159 | **** |
| 160 | **** |
| 161 | **** |
| 162 | **** |
| 163 | **** |
| 164 | **** |
| 165 | *** |
| 166 | **** |
| 167 | **** |
| 168 | **** |
| 169 | **** |
| 170 | **** |
| 171 | ** |
| 172 | **** |
| 173 | *** |
| 174 | **** |
| 175 | ** |
| 176 | ** |
| 177 | * |
| 178 | *** |
| 179 | **** |
| 180 | **** |
| 181 | **** |
| 182 | *** |
| 183 | *** |
| 184 | ** |
| 185 | *** |
| 186 | *** |
| 187 | **** |
| 188 | *** |
| 189 | **** |
| 190 | **** |
| 191 | **** |
| 192 | **** |
| 193 | *** |
| 194 | **** |
| 195 | **** |
| 196 | **** |
| 197 | *** |
| 198 | **** |
| 199 | **** |
| 200 | *** |
| 201 | **** |

TABLE 3
USP19 inhibition by exemplified compounds. The USP19 inhibitory
activities are classified as the following:

| | **** | *** | ** | * |
|---|---|---|---|---|
| USP19 IC₅₀ | IC₅₀ < | 0.01 ≤ | 0.1 ≤ | 1 ≤ |
| [μM] | 0.01 | IC₅₀ < 0.1 | IC₅₀ < 1 | IC₅₀ < 20 |

USP19 activity was determined in a fluorescence polarisation (FP) homogeneous assay using the isopeptide Ubiquitin-Lys-TAMRA substrate (either AUB-101, Almac Sciences Scotland Limited, or U-558, Boston Biochem, both of which gave identical results). Full-length USP19 was purchased from Boston Biochem (E-576). Unless otherwise stated, all other reagents were purchased from Sigma. Enzymatic reactions were conducted in black flat bottom polystyrene 384-well plates (Nunc) and 30 μL total volume. USP19 (2.5 nM, 10 μL) was incubated in assay buffer (50 mM HEPES (pH 7.4), 150 mM NaCl, 5 mM DTT, 0.05% BSA (w/v), 0.05% CHAPS) in the presence or absence of inhibitor (10 μL). Inhibitors were stored as 10 mM DMSO stocks in an inert environment (low humidity, dark, low oxygen, rt) using the StoragePod® system (Roylan Developments) and serial dilutions were prepared in buffer just prior to the assay (from 200 μM to 2 μM, 8-18 data point curve). Following incubation at RT for 30 min, the enzymatic reactions were initiated by dispensing the Ub substrate (500 nM, 10 μL). FP was measured every 15 min over a period of 90 min (within the linear range of the assay) using a Synergy 4 plate reader (BioTek) exciting at 530 nm and measuring the amount of parallel and perpendicular light at 575 nm. The FP signal was subsequently normalised to the no compound control. Data were plotted and fitted, and the concentrations resulting in 50% inhibition (IC₅₀) were calculated using the non-linear regression curve fitting model using GraphPad (Prism). IC₅₀ values for the inhibitors of the invention are compiled in Tables 1 and 3 and represent the average of at least two duplicate experiments.

### Cellular Target Engagement Using Western Blotting

Cells from a breast cancer cell line, a neuroblastoma cell line and a mouse skeletal muscle cell line were treated with a USP19 inhibitor compound (ADC-141) for 2 hrs, lysed (lysis buffer: 50 mM Tris pH 7.4; 150 mM NaCl; 5 mM MgCl2; 0.5 mM EDTA; 0.5% NP40; 10% Glycerol; 2 mM DTT) and Ubiquitin-propargylamine (Ub-PA; UbiQ) or Ubiquitin-vinyl methyl ester (Ub-VME; Almac Sciences Scotland Limited) was then added. Samples were analysed by western blotting probing for USP19 (EC₅₀ determined by densitometry).

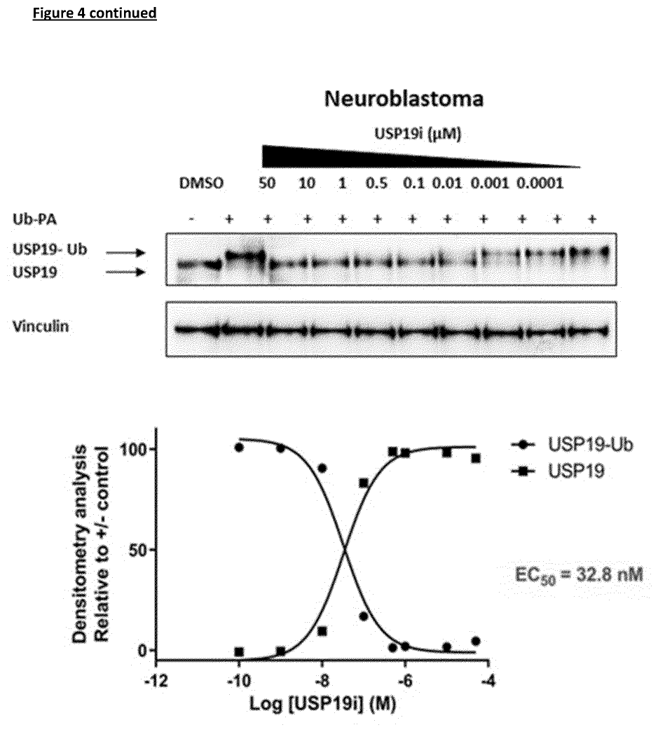

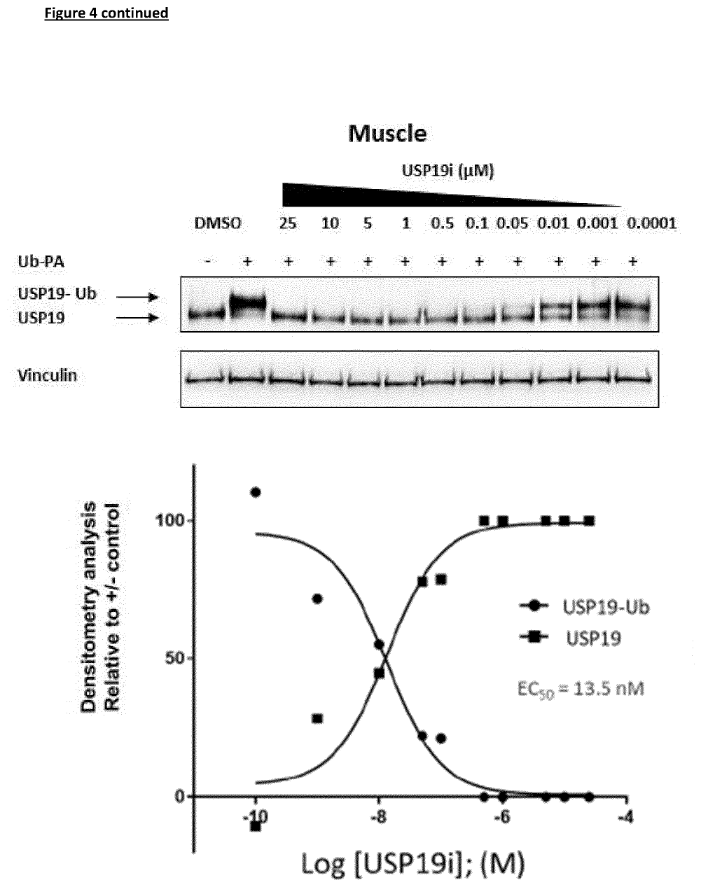

In each cell line, the USP19 inhibitor compound showed good cell permeability and exhibited a low nanomolar EC₅₀. The results for each cell line are shown in FIG. 4.

### Cellular Target Engagement Using HTRF Assay

USP19 cellular target engagement was determined in a homogenous time resolved fluorescence (HTRF) assay using HA-tagged ubiquitin vinyl pentynyl sulfone (VPS) probe (UbiQ-193, UbiQ Bio) and a USP19-Flag overexpression construct. Inhibitors were stored as 10 mM DMSO stocks in an inert environment (low humidity, dark, low oxygen, rt) using the StoragePod® system (Roylan Developments). USP19-Flag transfected cells were incubated with serially diluted compound in an 11 point dose response curve (from 50 μM to 0.01 nM) for 2 h and then washed in PBS and lysed. Unless otherwise stated, all HTRF reagents were purchased from CisBio. HTRF assays using cell lysates were completed in 384 well plates (Greiner) in a 20 μL total volume. Cell lysates were incubated with HA-tagged ubiquitin VPS probe for 40 min in PPI detection buffer prior to addition of anti-HA and anti-FLAG HTRF detection reagents. HTRF was measured every 1 h for 18 h using a Pherastar FSX plate reader (excitation of 337 nm, emission of 620/665 nm). Data was normalised to DMSO (no compound controls) and fitted using IC₅₀ values derived using Prism (GraphPad) using nonlinear regression curve fitting.

### Caco-2 Permeability Assay

Caco-2 cells are commonly used as an in vitro model for the prediction of human intestinal absorption of test compounds. When cultured, Caco-2 cells (derived from a human colorectal carcinoma) spontaneously differentiate into monolayers of polarised enterocytes. The cells are seeded on multiwell-insert plates and form a confluent monolayer over 20 d before the assay. On day 20, compound was added to the apical side of the membrane and the flux of the compound across the monolayer was monitored over 2 h. Only the data for the permeability coefficient in the apical to basolateral direction (Pₐₚₚ A:B) in shown in Table 1. All data was generated at Cyptotex.

### Thermodynamic Solubility Assay

Test compound (˜0.5 mg, accurately weighed) was suspended in PBS buffer pH 7.4 (Dulbecco A) to a concentration of 1 mg/mL in a high recovery glass vial in duplicate. The suspensions were shaken at 300 rpm at rt for 64 h. About 250 μL of suspension were then transferred to a MultiScreen® Solubility filter plate (Millipore) in duplicate. Concentrations of the filtrates were then quantified against a 5-point calibration curve in a mixture of acetonitrile/PBS buffer (top concentration 500 PM). After filtration and matrix match, the calibration and assay plates were analysed on a Bioteck Synergy 4 plate reader (240-400 nm). Final concentration of the test compound in the filtrate was calculated using the slope of the calibration curve.

### In Vivo Activity

The following data from an in vivo model is the first demonstration that a USP19 inhibitor can be used to treat muscle loss, to reduce fat deposition and to improve insulin sensitivity. These data demonstrate that compounds which potently inhibit USP19 activity can effectively treat muscular atrophy, obesity and/or insulin resistance.

### Methods:

To induce muscle wasting, a 1 cm segment of the sciatic nerve in the thigh was removed from mice (male C57bl/6 mice at 8-10 weeks of age; n=10 per group) under isoflurane anaesthesia and analgesia with carprofen. A sham operation was carried out in the opposite leg as a control.

Mice were randomised into Vehicle or Test groups, with all animals weighed to ensure a similar mean weight in each group. ADC-141, a USP19 inhibitory compound at 75 mg/kg or Vehicle was administered IP twice daily starting from the evening post-operation.

Mice were sacrificed 14 days later. Fat pads, liver, gastrocnemius and tibialis anterior muscles were harvested. Tissue mass were measured in both groups.

To assess obesity and insulin resistance, a diet-induced obesity mouse model was used. The diet-induced obese (DIO) mouse is a well characterised model of obesity which exhibits increased adiposity, insulin resistance and glucose intolerance.

Male C57BL6/J mice were continuously provided with high-fat diet (D12451, 45% kcal as fat; Research Diets, New Jersey, USA) and filtered tap water ad libitum for the duration of the study. From day 0, mice were administered vehicle i.p. BID, USP19 inhibitor (ADC-141) i.p. BID at 5 mg/kg or 25 mg/kg, or positive control liraglutide 0.1 mg/kg s.c. BID.

Body weight was measured daily. On Day 13, body composition was be assessed by DEXA. On Day 15, fasting glucose and insulin levels were measured before and during an oral glucose tolerance test (OGTT) to assess improvements in glucose control. The OGTT was performed following an overnight fast. Hence, on Day 14 food (but not water) was removed beginning at approximately 16:45, immediately after the PM dose. An OGTT was performed the following morning (approx. 16 h post fast). Mice were dosed with vehicle or test compound (starting at 08.45) to a timed schedule 30 minutes prior to the administration of the glucose challenge (2.0 g/kg po). Blood samples were taken immediately prior to dosing (B1), immediately prior to glucose administration (B2) and 15, 30, 60 and 120 minutes after glucose administration.

ADC-141 is 1-(((S)-7-((R)-3-cyclohexyl-2-methylpropanoyl)-10-hydroxy-7-azaspiro[4.5]decan-10-yl)methyl)-4-phenyl-5-(piperazine-1-carbonyl)pyridin-2(1H)-one, corresponding to exemplary compound 212 provided in WO2018/020242. Both ADC-141 and the compounds provided herein are shown to have USP19 inhibitory activity using the fluorescence polarisation assay described above. It is therefore expected that the USP19 inhibitor compounds provided herein will show levels of efficacy similar to that described below for ADC-141.

### Results:

### Muscular Atrophy

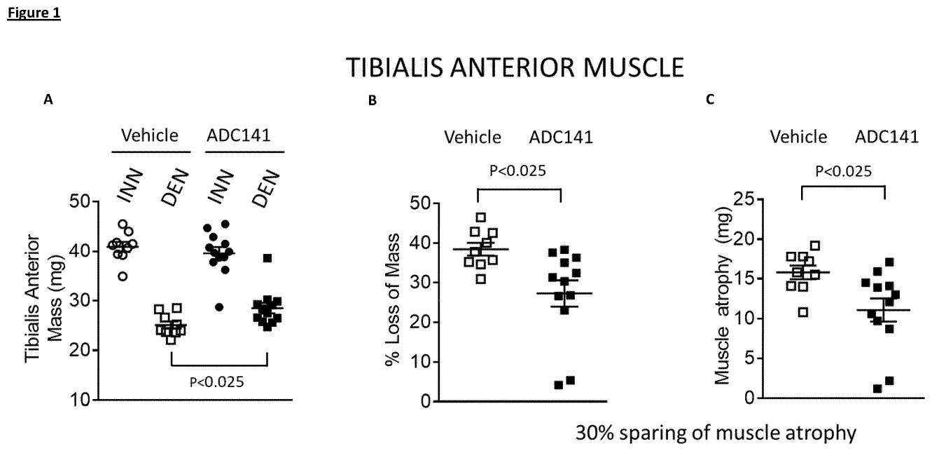

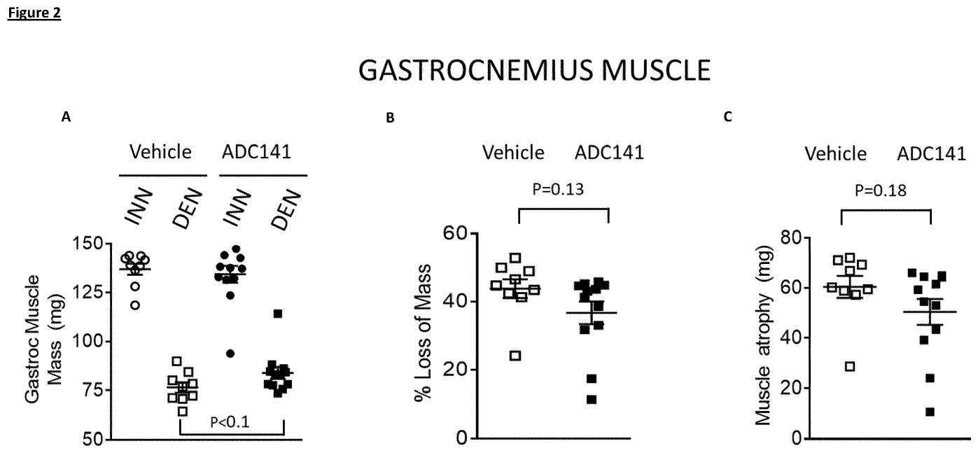

As shown in FIG. 1, mice receiving a USP19 inhibitor had a significantly lower loss of muscle mass in the tibialis anterior muscle compared to mice receiving vehicle only. The sparing of muscle atrophy was evident both in terms of percentage mass (FIG. 1B) and absolute muscle mass (FIG. 1C).

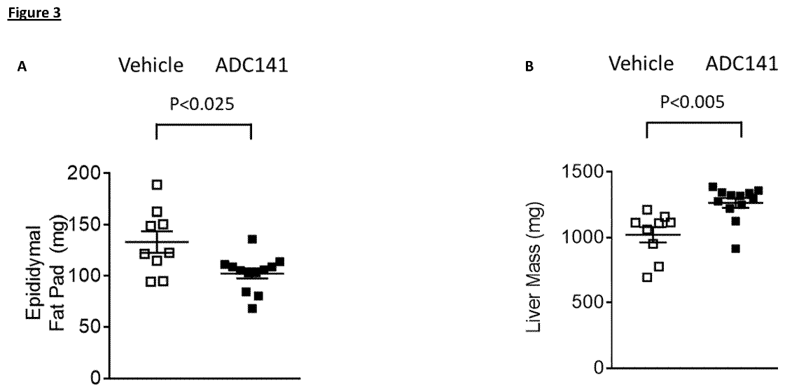

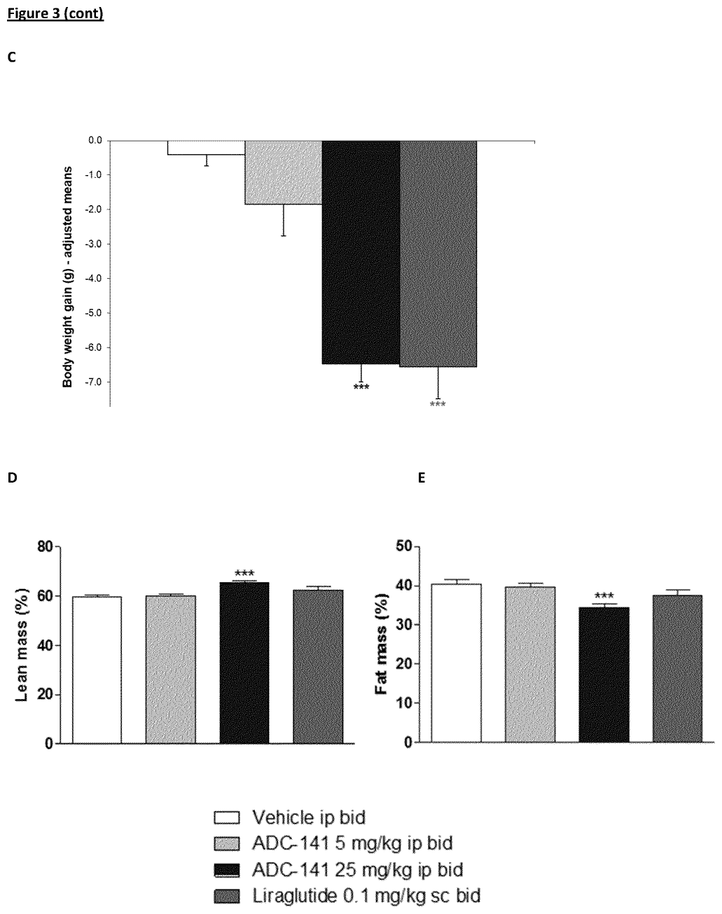

Muscle wasting was also reduced in the gastrocnemius muscle (FIG. 2), though the trend did not reach significance. Again, mice receiving a USP19 inhibitor exhibited less muscle wasting both in terms of percentage mass (FIG. 2B) and absolute muscle mass (FIG. 2C).

These data demonstrate the pharmacological inhibition of USP19 in vivo can reduce muscular atrophy. The data indicate that pharmacological inhibition of USP19 will be especially effective at reducing muscle wasting as a result of inactivity, immobilisation or other disuse. On the basis of the results provided herein, pharmacological USP19 inhibition is also expected to be effective in treating muscular atrophy as a result of cachexia or sarcopenia.

### Obesity

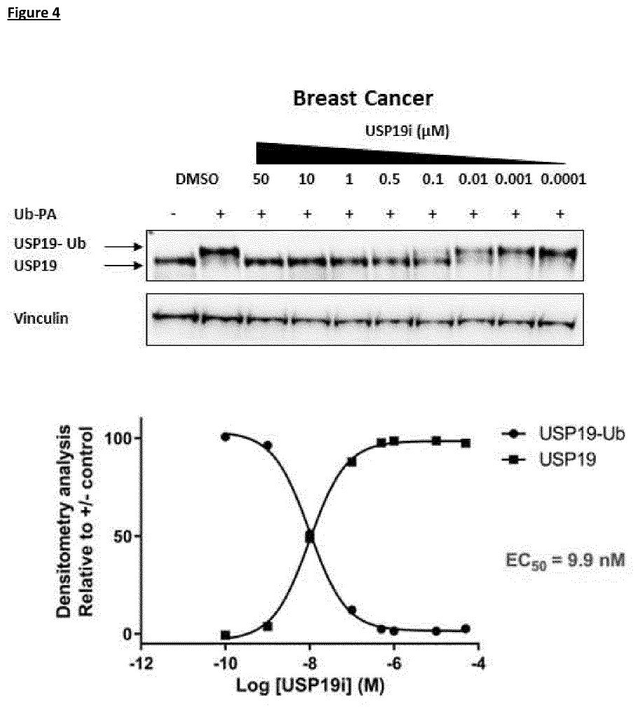

FIG. 3A shows the mass of the epididymal fat pad in mice following 2 weeks of receiving a USP19 inhibitor or vehicle alone. As shown in FIG. 3, mice which received the USP19 inhibitor had significantly smaller fat pads compared to vehicle treated mice.

FIG. 3B shows an increase in liver mass in mice treated with a USP19 inhibitor. This is thought to be as a result of drug accumulation in the liver.

FIG. 3C shows that mice receiving USP19 inhibitor exhibited a reduction in overall body weight gain when on a high-fat diet. FIGS. 3D and 3E show that this is due to a reduction in fat mass, but that lean body mass is preserved. DIO mice treated with USP19 inhibitor also exhibited a reduction in cumulative food intake compared to vehicle control mice.

The data shown in FIG. 3 is the first demonstration that pharmacological inhibition of USP19 can reduce fat accumulation in a wild-type background. Gene knockout studies have described a possible association between USP19 and fat accumulation (Coyne et al, Diabetologia. 2018 Nov. 1. doi: 10.1007/s00125-018-4754-4., incorporated herein by reference). However, acute or chronic pharmacological inhibition of an enzyme does not always result in similar physiological outcomes to genetic ablation.

The in vivo pharmacological inhibition data provided herein demonstrate that compounds which potently inhibit USP19 activity can effectively treat obesity.

### Insulin Resistance

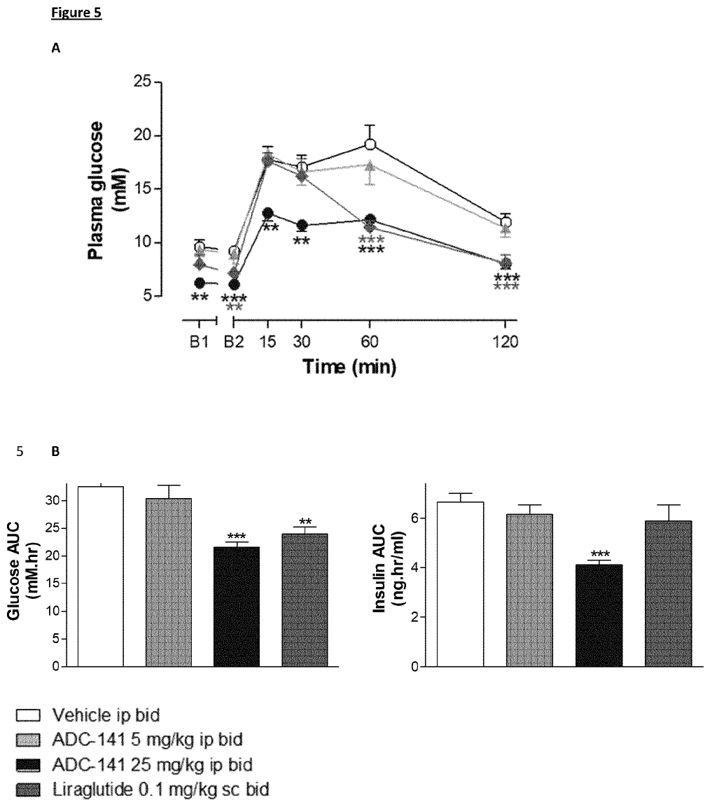

FIG. 5 shows the results of an oral glucose tolerance test (OGTT) in mice with diet-induced obesity. Untreated mice exhibit the symptoms of insulin-resistance characterised by elevated plasma glucose and plasma insulin levels. Mice treated with a USP19 inhibitor exhibit a dose-dependent improvement in OGTT response characterised by decreased plasma glucose and decreased plasma insulin.

The data shown in FIG. 5 is the first demonstration that pharmacological inhibition of USP19 can reduce insulin resistance in a wild-type background. Gene knockout studies have also described an association between USP19 and insulin sensitivity (Coyne et al, supra). Coyne et al. describe an improvement in insulin sensitivity in USP19 knockout mice but, as noted above, it could not be assumed that the effects would translate to pharmacological inhibition of USP19 in wild-type subjects.

The data provided herein is the first demonstration that pharmacological inhibition of USP19 effectively treats insulin resistance.

The data presented herein is the first demonstration of the therapeutic effects of pharmacological inhibition of USP19. Accordingly, the USP19 inhibitor compounds provided herein can effectively treat muscular atrophy, obesity and/or insulin resistance.

### EXPERIMENTAL SECTION

### Abbreviations and Acronyms

AcOH: acetic acid; aq: aqueous; atm: atmosphere(s); dba: dibenzylideneacetone; Bn: benzyl; Boc: tert-butyloxycarbonyl; br: broad; CAN: ceric ammonium nitrate; Cbz: carboxybenzyl; CDI: carbonyldiimidazole; DBU: 1,8-diazabicyclo[5.4.0]undec-7-ene; DCM: dichloromethane; d: doublet (spectral); de: diastereomeric excess; DIPEA: diisopropylethylamine; DMA: N,N-dimethylacetamide; DMAP: 4-dimethylaminopyridine; DME: dimethoxyethane; DMF: N,N-dimethylformamide; DMSO: dimethylsulfoxide; dppf: 1,1′-bis(diphenylphosphino)ferrocene; Eaton's reagent: phosphorus pentoxide, 7.7 wt % in methanesulfonic acid; EDC: N-(3-dimethylaminopropyl)-N-ethylcarbodiimide hydrochloride; ee: enantiomeric excess; equiv.: equivalents; EtOAc: ethyl acetate; EtOH: ethanol; Ex.: Example; PE: petroleum ether 40/60; ESI: electrospray ionisation; h: hour(s); GCMS: gas chromatography mass spectrometry; HATU: N-[(dimethylamino)-1H-1,2,3-triazolo-[4,5-b]pyridin-1-ylmethylene]-N-methylmethanaminium hexafluorophosphate N-oxide; hept: heptet (spectral); HPLC: high pressure liquid chromatography; IPA: 2-propanol; LC: liquid chromatography; LCMS: liquid chromatography mass spectrometry; LiHMDS: lithium bis(trimethylsilyl)amide; M: molar; m/z: mass-to-charge ratio; mCPBA: 3-chloroperbenzoic acid; MeCN: acetonitrile; MeOH: methanol; min: minute(s); mmol: millimole(s); MS: mass spectrometry; MTBE: methyl tert-butyl ether; m: multiplet (spectral); NaHMDS: sodium bis(trimethylsilyl)amide; NMP: N-methyl-2-pyrrolidone; NMR: nuclear magnetic resonance; p: pentet (spectral); Ph: phenyl; PMB: p-methoxybenzyl; ppm: parts per million; q: quartet (spectral); quint: quintet (spectral); RBF: round-bottom flask; R_{T}: retention time; rt: room temperature; s: singlet; SCX: strong cation exchange; SFC: supercritical fluid chromatography; SM: starting material; TBDMS: tert-butyldimethylsilyl; Teoc: 2-(trimethylsilyl)ethoxycarbonyl; TFA: trifluoroacetic acid; THF: tetrahydrofuran; t: triplet; UV: ultraviolet; v/v: volume per unit volume; wt %: weight percent; w/v: weight per unit volume; w/w: weight per unit weight; Xantphos: 4,5-bis(diphenylphosphino)-9,9-dimethylxanthene; XPhos: 2-dicyclohexylphosphino-2′,4′,6′-triisopropylbiphenyl.

### General Experimental Conditions

### Solvents and Reagents

Common organic solvents that were used in reactions (e.g. THF, DMF, DCM, and MeOH) were purchased anhydrous from Sigma-Aldrich® in Sure/Seal™ bottles and were handled appropriately under nitrogen. Water was deionised using an Elga PURELAB Option-Q. All other solvents used (i.e. for work-up procedures and purification) were generally HPLC grade and were used as supplied from various commercial sources. Unless otherwise stated, all starting materials used were purchased from commercial suppliers and used as supplied.

### Microwave Synthesis

Microwave experiments were carried out using a Biotage Initiator™ Eight instrument. The system gives good reproducibility and control at temperature ranges from 60-250° C. and pressures of up to a maximum of 20 bar.

### Flash Chromatography

Purification of compounds by flash chromatography was achieved using a Biotage Isolera Four system. Unless otherwise stated, Biotage KP-Sil SNAP cartridge columns (10-340 g) or Grace GraceResolv cartridge columns (4-330 g) were used along with the stated solvent system and an appropriate solvent gradient depending on compound polarity. In the case of more polar and basic compounds, Biotage KP-NH SNAP cartridge columns (11 g) were used.

### NMR Spectroscopy

¹H NMR spectra were recorded at ambient temperature using a Bruker Avance (300 MHz), Bruker Avance III (400 MHz) or Bruker Ascend (500 MHz) spectrometer. All chemical shifts (δ) are expressed in ppm. Residual solvent signals were used as an internal standard and the characteristic solvent peaks were corrected to the reference data outlined in J. Org. Chem., 1997, 62, p 7512-7515; in other cases, NMR solvents contained tetramethylsilane, which was used as an internal standard.

### Liquid Chromatography Mass Spectrometry (LCMS)

Liquid Chromatography Mass Spectrometry (LCMS) experiments to determine retention times (R_{T}) and associated mass ions were performed using the following methods:

Method A: The system consisted of an Agilent Technologies 6130 quadrupole mass spectrometer linked to an Agilent Technologies 1290 Infinity LC system with UV diode array detector and autosampler. The spectrometer consisted of an electrospray ionization source operating in positive and negative ion mode. LCMS experiments were performed on each sample submitted using the following conditions: LC Column: Agilent Eclipse Plus C18 RRHD, 1.8 μm, 50×2.1 mm maintained at 40° C. Mobile phases: A) 0.1% (v/v) formic acid in water; B) 0.1% (v/v) formic acid in acetonitrile.

| Gradient Time (min) | Flow (mL/min) | % A | % B |
|---|---|---|---|
| 0.00 | 0.5 | 80 | 20 |
| 1.80 | 0.5 | 0 | 100 |
| 2.20 | 0.5 | 0 | 100 |
| 2.50 | 0.5 | 80 | 20 |
| 3.00 | 0.5 | 80 | 20 |

Method B: The system consisted of an Agilent Technologies 6140 single quadrupole mass spectrometer linked to an Agilent Technologies 1290 Infinity LC system with UV diode array detector and autosampler. The spectrometer consisted of a multimode ionization source (electrospray and atmospheric pressure chemical ionizations) operating in positive and negative ion mode. LCMS experiments were performed on each sample submitted using the following conditions: LC Column: Zorbax Eclipse Plus C18 RRHD, 1.8 μm, 50×2.1 mm maintained at 40° C. Mobile phases: A) 0.1% (v/v) formic acid in water; B) 0.1% (v/v) formic acid in acetonitrile.

| Gradient Time (min) | Flow (mL/min) | % A | % B |
|---|---|---|---|
| 0.00 | 1.0 | 95 | 5 |
| 1.80 | 1.0 | 0 | 100 |
| 2.20 | 1.0 | 0 | 100 |
| 2.21 | 1.0 | 95 | 5 |
| 2.50 | 1.0 | 95 | 5 |

Method C: The system consisted of Shimadzu Prominence HPLC/Applied Biosystem LCMS/MS API 2000 instruments. Spectrometer ionization technique: ESI using API source operating in positive ion mode. LCMS experiments were performed on each sample submitted using the following conditions: LC Column: XBridge C18, 5 μm, 4.6×50 mm maintained at 25° C. Mobile phases: A) 10 mM ammonium acetate (aq); B) acetonitrile.

| Gradient Time (min) | Flow (mL/min) | % A | % B |
|---|---|---|---|
| 0.01 | 1.2 | 90 | 10 |
| 1.50 | 1.2 | 70 | 30 |
| 3.00 | 1.2 | 10 | 90 |
| 4.00 | 1.2 | 10 | 90 |
| 5.00 | 1.2 | 90 | 10 |

Method D: The system consisted of Shimadzu Prominence HPLC/Applied Biosystem LCMS/MS API 2000 instruments. Spectrometer ionization technique: ESI using API source operating in positive ion mode. LCMS experiments were performed on each sample submitted using the following conditions: LC Column: Zorbax Extend C18, 5 μm, 4.6×50 mm maintained at 25° C. Mobile phases: A) 10 mM ammonium acetate (aq); B) acetonitrile.

| Gradient Time (min) | Flow (mL/min) | % A | % B |
|---|---|---|---|
| 0.01 | 1.2 | 90 | 10 |
| 1.50 | 1.2 | 70 | 30 |
| 3.00 | 1.2 | 10 | 90 |
| 4.00 | 1.2 | 10 | 90 |
| 5.00 | 1.2 | 90 | 10 |

Method E: The system consisted of Waters ACQUITY UPLC/Waters ACQUITY SQD mass spectrometer instruments. Spectrometer ionization technique: ESI operating in positive ion mode. LCMS experiments were performed on each sample submitted using the following conditions: LC Column: YMC Triart C18, 3 μm, 2.1×33 mm maintained at 50° C. Mobile phases: A) 0.05% (v/v) formic acid in water; B) acetonitrile.

| Gradient Time (min) | Flow (mL/min) | % A | % B |
|---|---|---|---|
| 0.00 | 1.0 | 98 | 2 |
| 0.75 | 1.0 | 98 | 2 |
| 1.00 | 1.0 | 90 | 10 |
| 2.00 | 1.0 | 2 | 98 |
| 2.25 | 1.0 | 2 | 98 |
| 2.90 | 1.0 | 98 | 2 |
| 3.00 | 1.0 | 98 | 2 |

Method F: The system consisted of either an Agilent Technologies 1100 Series LC/MSD system with UV diode array detector and evaporative light scattering detector (DAD/ELSD) and Agilent LC/MSD VL (G1956 A), SL (G1956B) mass spectrometer or an Agilent 1200 Series LC/MSD system with DAD/ELSD and Agilent LC/MSD SL (G6130 A), SL (G6140 A) mass spectrometer. All of the LCMS data were obtained using the atmospheric pressure chemical ionization mode with positive and negative ion mode switching with a scan range of m/z 80-1000. LCMS experiments were performed on each sample submitted using the following conditions: LC Column: Zorbax SB-C18 RRHD, 1.8 μm, 4.6×15 mm. Mobile phases: A) 0.1% (v/v) formic acid in water; B) 0.1% (v/v) formic acid in acetonitrile.

| Gradient Time (min) | Flow (mL/min) | % A | % B |
|---|---|---|---|
| 0.00 | 3.0 | 100 | 0 |
| 1.50 | 3.0 | 0 | 100 |
| 1.80 | 3.0 | 0 | 100 |
| 1.81 | 3.0 | 100 | 0 |

Method G: The system consisted of Waters ACQUITY UPLC/Waters ACQUITY SQD mass spectrometer instruments. Spectrometer ionization technique: ESI operating in positive ion mode. LCMS experiments were performed on each sample submitted using the following conditions: LC Column: Luna Omega, 3 μm, 4.6×100 mm maintained at 50° C. Mobile phases: A) 0.05% (v/v) TFA in water; B) acetonitrile.

| Gradient Time (min) | Flow (mL/min) | % A | % B |
|---|---|---|---|
| 0.00 | 1.0 | 98 | 2 |
| 1.00 | 1.0 | 98 | 2 |
| 5.00 | 1.0 | 50 | 50 |
| 9.00 | 1.0 | 5 | 95 |
| 11.00 | 1.0 | 5 | 95 |
| 12.00 | 1.0 | 98 | 2 |

Method H: The system consisted of Waters ACQUITY H Class UPLC/Waters ACQUITY SQD 2 mass spectrometer instruments. Spectrometer ionization technique: ESI operating in positive ion mode. LCMS experiments were performed on each sample submitted using the following conditions: LC Column: XBridge C18, 3.5 μm, 3×50 mm maintained at 50° C. Mobile phases: A) 5 mM ammonium acetate (aq); B) 9:1 5 mM ammonium acetate in acetonitrile/water.

| Gradient Time (min) | Flow (mL/min) | % A | % B |
|---|---|---|---|
| 0.00 | 1.20 | 95 | 5 |
| 0.75 | 1.20 | 95 | 5 |
| 1.00 | 1.20 | 70 | 30 |
| 2.00 | 1.20 | 2 | 98 |
| 2.50 | 1.20 | 2 | 98 |
| 2.75 | 1.20 | 95 | 5 |
| 3.00 | 1.20 | 95 | 5 |

Method I: The system consisted of Waters ACQUITY H Class UPLC/Waters ACQUITY SQD 2 mass spectrometer instruments. Spectrometer ionization technique: ESI operating in positive ion mode. LCMS experiments were performed on each sample submitted using the following conditions: LC Column: Waters Acquity UPLC BEH C8, 1.7 μm, 2.1×50 mm maintained at 50° C. Mobile phases: A) 0.05% (v/v) formic acid in water; B) 9:1 0.05% (v/v) formic acid in acetonitrile/water.

| Gradient Time (min) | Flow (mL/min) | % A | % B |
|---|---|---|---|
| 0.00 | 0.80 | 90 | 10 |
| 0.75 | 0.80 | 90 | 10 |
| 1.00 | 0.80 | 50 | 50 |
| 2.00 | 0.80 | 2 | 98 |
| 2.25 | 0.80 | 2 | 98 |
| 2.60 | 0.80 | 90 | 10 |
| 3.00 | 0.80 | 90 | 10 |

Method J: The system consisted of Waters ACQUITY H Class UPLC/Waters ACQUITY SQD 2 mass spectrometer instruments. Spectrometer ionization technique: ESI operating in positive ion mode. LCMS experiments were performed on each sample submitted using the following conditions: LC Column: Waters Acquity UPLC BEH C8, 1.7 μm, 2.1×30 mm maintained at 50° C. Mobile phases: A) 5 mM ammonium acetate (aq); B) 9:1 5 mM ammonium acetate in acetonitrile/water.

| Gradient Time (min) | Flow (mL/min) | % A | % B |
|---|---|---|---|
| 0.00 | 0.50 | 98 | 2 |
| 0.50 | 0.50 | 98 | 2 |
| 1.50 | 0.50 | 2 | 98 |
| 2.50 | 0.50 | 2 | 98 |
| 2.75 | 0.50 | 98 | 2 |
| 3.00 | 0.50 | 98 | 2 |

Method K: The system consisted of Waters ACQUITY H Class Plus UPLC/Waters ACQUITY QDa mass spectrometer instruments with UV detector and autosampler. Spectrometer ionization technique: ESI operating in positive ion mode and negative. LCMS experiments were performed on each sample submitted using the following conditions: LC Column: Agilent Extend-C18 RRHD, 1.8 μm, 2.1×50 mm maintained at 40° C. Mobile phases: A) 0.1% (v/v) formic acid in water; B) 0.1% (v/v) formic acid in acetonitrile.

| Gradient Time (min) | Flow (mL/min) | % A | % B |
|---|---|---|---|
| 0.00 | 0.80 | 95 | 5 |
| 1.80 | 0.80 | 5 | 95 |
| 2.20 | 0.80 | 5 | 95 |
| 2.30 | 0.80 | 95 | 5 |
| 2.50 | 0.80 | 95 | 5 |

### Gas Chromatography Mass Spectrometry (GCMS)

Method A: The system consisted of Agilent 7890B GC and Agilent 5977B GC/MSD instruments. GCMS experiments were performed on each sample submitted using the following conditions: GC Column: HP-5 ms (30 m×0.25 mm, 0.25 μm). Carrier gas: Helium. Inlet temperature: 250° C. Split ratio: 20:1. Carrier gas flow: 1.0 mL/min. Ramp profile: Oven temperature initially 60° C. held for 2 min, increasing to 100° C. over 2 min (20° C./min) and held for 2 min, then increasing to 310° C. over 5.25 min (40° C./min), then held for 4 min (total run time: 15.25 min).

### Preparative High Pressure Liquid Chromatography

The system consisted of an Agilent Technologies 6120 single quadrupole mass spectrometer linked to an Agilent Technologies 1200 Preparative LC system with multiple wavelength detector and autosampler. The mass spectrometer used a multimode ionization source (electrospray and atmospheric pressure chemical ionizations) operating in positive and negative ion mode. Fraction collection was mass-triggered (multimode positive and negative ion). Purification experiments, unless otherwise stated, were performed under basic conditions at an appropriate solvent gradient that was typically determined by the retention time found using the LCMS method. In cases where the basic conditions were unsuccessful, acidic conditions were employed.

Basic conditions: LC Column: Waters XBridge™ Prep C18 5 μm OBD™ 30×100 mm column at rt. Mobile phase: A) 0.1% (v/v) ammonium hydroxide in water; B) 0.1% (v/v) ammonium hydroxide in 95:5, acetonitrile/water. Total experiment time was ca. 10 min and a generic method is shown:

| Gradient Time (min) | Flow (mL/min) | % A | % B |
|---|---|---|---|
| 0.00 | 20.0 | 50 | 50 |
| 3.00 | 20.0 | 12 | 88 |
| 5.00 | 20.0 | 12 | 88 |
| 7.00 | 20.0 | 0 | 100 |
| 8.0 | 20.0 | 0 | 100 |
| 8.20 | 20.0 | 50 | 50 |

### Chiral Separation of Stereoimers by Supercritical Fluid Chromatography (SFC)

The separation of mixtures of stereoisomers was performed using the following general procedure. The mixture of stereoisomers was dissolved to 50 mg/mL in methanol and purified by SFC under the stated conditions. Combined fractions of each of stereoisomer were evaporated to near dryness using a rotary evaporator, transferred into final vessels using DCM, which was removed under a stream of compressed air at 40° C., before being stored in a vacuum oven at 40° C. and 5 mbar for 16 h.

### Chiral Separation of Stereoimers by HPLC

The separation of mixtures of stereoisomers was performed using the following general procedure. The mixture of stereoisomers was dissolved to 66 mg/mL in methanol and purified by HPLC under the stated conditions. Combined fractions of each of stereoisomer were evaporated to near dryness using a rotary evaporator, transferred into final vessels using MeOH, which was removed under a stream of compressed air at 35° C., before being stored in a vacuum oven at 35° C. and 5 mbar for 16 h.

### Chiral Purity Analysis

After chiral separation of mixtures of stereoisomers, each stereoisomer was analysed to determine chiral purity using the following analytical SFC or HPLC methods under the stated conditions.

### Method A (SFC):

| Column Details | Chiralpak IG (4.6 mm × 250 mm, 5 μm) | |
|---|---|---|
| Column Temperature | 40° | C. |
| Flow Rate | 4 | mL/min |
| BPR | 125 | BarG |
| Detector Wavelength | 210-400 | nm |
| Injection Volume | 1 | μL |

| | |
|---|---|
| Isocratic Conditions | 30:70 MeOH:CO₂ (0.2% v/v NH₃) |

### Method B (HPLC):

| Column Details | Lux A2 (4.6 mm × 250 mm, 5 μm) | |
|---|---|---|
| Column Temperature | Ambient | |
| Flow Rate | 1 | mL/min |
| Detector Wavelength | 254 | nm |
| Injection Volume | 1 | μL |

| | |
|---|---|
| Isocratic Conditions | 50:50 heptane:IPA (0.2% v/v NH₃) |

### Method C (HPLC):

| Column Details | Lux C1 (4.6 mm × 150 mm, 5 μm) | |
|---|---|---|
| Column Temperature | Ambient | |
| Flow Rate | 1 | mL/min |
| Detector Wavelength | 254 | nm |
| Injection Volume | 1 | μL |

| | |
|---|---|
| Isocratic Conditions | MeOH (0.2% v/v NH₃) |

### Method D (SFC):

| Column Details | Lux C1 (4.6 mm × 250 mm, 5 μm) | |
|---|---|---|
| Column Temperature | 40° | C. |
| Flow Rate | 4 | mL/min |
| Detector Wavelength | 210-400 | nm |
| Injection Volume | 1 | μL |
| BPR | 125 | BarG |

| | |
|---|---|
| Isocratic Conditions | 50:50 MeOH:CO₂ (0.2% v/v NH₃) |

### Method E (SFC):

| Column Details | Lux C4 (4.6 mm × 150 mm, 5 μm) | |
|---|---|---|
| Column Temperature | 40° | C. |
| Flow Rate | 4 | mL/min |
| Detector Wavelength | 210-400 | nm |
| Injection Volume | 1 | μL |
| BPR | 125 | BarG |

| | |
|---|---|
| Isocratic Conditions | 60:40 MeOH:CO₂ (0.2% v/v NH₃) |

### Nomenclature

Unless otherwise indicated, the nomenclature of structures was determined using the ‘Convert Structure to Name’ function of ChemDraw Professional 17.1 for Examples 1 to 58 and ChemDraw Professional 20.1 for Examples 59 to 201 (CambridgeSoft/PerkinElmer). In the cases in which R² and R³ are not H and R⁰ is chiral, the active conformation has been tentatively assigned with the R⁰ bond as “up” with respect to the structure drawn in formula (I). This is inferred from previous observations that showed that this was the case when R⁰═OH and X-ray crystallography data was used for confirmation (WO2018020242). Hence, when R⁰═H, the active conformation at that stereocentre is assigned (R)-configuration and when R⁰═F, NH2 or OMe, the active conformation at that stereocentre is assigned (S)-configuration in the following Examples in which R⁰ is chiral. However, it should be noted that for all of these Examples, it may be the case that they have been assigned with the incorrect configuration at the tertiary alcohol position due to an error in the determination of the original X-ray crystallography data or in the strategy of inferring the stereochemistry from other compounds. Therefore, it is possible that these compounds have the opposite configuration at this position. Both (R)- and (S)-enantiomers are disclosed herein, with the most potent preferred.

### General Procedures

### General Procedure 1: Boc Deprotection to Free Base

The Boc protected amine (1 equiv.) was dissolved in DCM and TFA or 4 M HCl in 1,4-dioxane was added. The reaction was stirred at rt for 1-24 h. The mixture was loaded onto a pre-equilibrated SCX-2 cartridge. The column was washed with a 4:1 mixture of DCM/MeOH and the basic compound was eluted using a 4:1 mixture of DCM/7 M NH₃ in MeOH. The ammoniacal fractions were concentrated in vacuo to give the desired product.

### General Procedure 2: Urea Formation Via a Carbamoyl Chloride Intermediate

To a solution of triphosgene (0.3-0.6 equiv.) in MeCN at 0° C. was added pyridine or DIPEA (2-5 equiv.) and the solution stirred for 10 min. A solution of the appropriate first amine (1 equiv.) in MeCN was added and the reaction stirred while warming to rt for 1-24 h. The mixture was added to the appropriate second amine (1 equiv.) followed by addition of DIPEA (2-5 equiv.) and stirred for a further 1-24 h. Saturated NaHCO_{3(aq)} was added. The resulting mixture was extracted with DCM (×3) using a phase separator, the combined organic phases were concentrated under reduced pressure and the residue was purified by flash chromatography to give the desired product.

### General Procedure 3: HATU Coupling

The appropriate amine (1 equiv.), carboxylic acid (1.0-1.5 equiv.) and HATU (1-1.5 equiv.) were dissolved in DCM and DIPEA (1-4 equiv.) was added. The reaction was stirred for 1-24 h before being quenched by the addition of saturated NaHCO_{3(aq)}. The resulting mixture was extracted with DCM (×3) using a phase separator. The combined organic extracts were concentrated under reduced pressure and the remaining residue was purified by flash chromatography to give the product.

### General Procedure 4: Alkylation of Nucleophiles Using Alkyl Halides

To a solution of the appropriate nucleophile (1 equiv.) in 1,4-dioxane was added the appropriate alkyl halide (1.1 equiv.) and cesium carbonate (2.0 equiv.). The reaction mixture was heated at 100° C. for 40 h. After cooling, the reaction mixture was filtered and evaporated to dryness. The remaining residue was purified by flash chromatography to give the desired product.

### General Procedure 5: Suzuki Coupling

A reaction vial was charged with a mixture of the appropriate halide (1 equiv.), the organoboron reagent (1-3 equiv.), a Pd catalyst (0.05-0.1 equiv.) and an inorganic base (2-5 equiv.) in a mixture of water and 1,4-dioxane or toluene, as stated. The mixture was degassed by evacuating and refilling with N₂ three times or by bubbling N₂ through for 5-15 min, then the reaction tube was sealed. The reaction was heated under the indicated conditions for the indicated time and allowed to cool to rt. Water or saturated NH₄Cl_{(aq)} was added and the resulting mixture was extracted using DCM (×3). The combined organic extracts were dried (phase separator), concentrated under reduced pressure and the remaining residue was purified by flash chromatography to give the desired product.

### General Procedure 6: Trifluoroacetamide Deprotection to Free Base

The appropriate trifluoroacetamide (1 equiv.) was dissolved in MeOH and water (10:1) and potassium carbonate (5 equiv.) was added. The resulting mixture was stirred at rt or up to 50° C. for 1-18 h. The reaction mixture was diluted with DCM and loaded onto a pre-equilibrated SCX-2 cartridge. The column was washed with a 4:1 mixture of DCM/MeOH and the basic compound was eluted using a 4:1 mixture of DCM/7 M NH₃ in MeOH. The ammoniacal fractions were concentrated in vacuo and further purified by flash chromatography (typically 0-20% MeOH in DCM or 0-20% MeOH in EtOAc) to give the desired product.

### General Procedure 7: Urea Formation Using a Carbamoyl Chloride Intermediate

The appropriate carbamoyl chloride (1-2 equiv.), amine or amine·HCl salt (1-3 equiv.) and DIPEA (2-6 equiv.) were stirred in the stated solvent at rt for 1-18 h before quenching with 0.5 M HCl_{(aq)} and extracted with DCM (×3) using a phase separator. The combined organic phases were concentrated in vacuo and the residue was purified by flash chromatography to give the desired product.

### General Procedure 8: Carbamoyl Chloride Formation

To a stirred solution of triphosgene (1 equiv.) in DCM was added pyridine (10 equiv.) dropwise at 0° C. After 30 min, a solution of the appropriate amine (1 equiv.) in DCM, or amine salt (1 equiv.) and DIPEA (1.5 equiv.) in DCM was added dropwise at 0° C. The temperature was allowed to increase to rt over 1-18 h. The reaction mixture was quenched by addition of 1 M HCl_{(aq)} and the resultant mixture was extracted with DCM (×3) using a phase separator. The combined organic phase was concentrated in vacuo to give the desired product without further purification.

### General Procedure 9: PMB Deprotection to Free Base

The PMB protected amine (1 equiv.) was dissolved in MeCN and cerium ammonium nitrate aqueous solution (4 equiv.) was added dropwise to the stirred solution at 0° C. The temperature was allowed to increase to rt. After 18 h, the volatiles were removed in vacuo and the remaining aqueous solution was basified by excess K₂CO₃ and extracted by MTBE. The solvents were removed in vacuo and the remaining residue was purified by flash chromatography or preparative HPLC to give the desired product.

### General Procedure 10: Teoc Deprotection to Free Base

The Teoc-protected amine (1 equiv.) was dissolved in DCM and TFA was added (typically 2:1, DCM/TFA by volume). The reaction was stirred at rt for 0.5-24 h before loading onto a pre-equilibrated SCX-2 cartridge. The column was washed with a 3:1 mixture of DCM/MeOH and the basic compound was eluted using a 3:1 mixture of DCM/7 M NH₃ in MeOH. The ammoniacal fractions were concentrated in vacuo and further purified by flash chromatography to give the desired product.

### Intermediate 1: tert-Butyl ((2S,4R)-2-phenylpiperidin-4-yl)carbamate

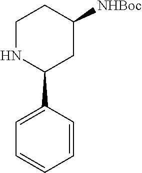 [SMILES: CC(C)(C)OC(=O)N[C@@H]1CCN[C@H](c2ccccc2)C1]

Step 1: Methyl (S)-3-amino-3-phenylpropanoate hydrochloride: To a stirred solution of (S)-3-amino-3-phenylpropanoic acid (144 g, 873 mmol) in MeOH (1.44 L) was added dropwise SOCl₂ (76 mL, 1047 mmol) at 0° C. and stirred at rt for 16 h. The reaction mixture was concentrated under reduced pressure and the residue was triturated with pentane and dried under vacuum to get title compound (156 g, 87%). LCMS (Method C): R_{T}=2.23 min, m/z=180 [M+H]⁺. ¹H NMR (400 MHz, DMSO-d₆): δ 8.75 (br s, 3H), 7.54 (d, 2H), 7.43-7.35 (m, 3H), 4.57 (br s, 1H), 3.54 (s, 3H), 3.24-31.6 (m, 1H), 3.03-2.97 (m, 1H).

Step 2: Methyl (S)-3-((3-methoxy-3-oxopropyl)amino)-3-phenylpropanoate: Methyl (S)-3-amino-3-phenylpropanoate hydrochloride (165 g, 766 mmol) was dissolved in MeOH (1.65 L). A solution of triethylamine (160 mL, 1149 mmol) in MeOH (1.48 L) was added followed by a solution of methyl acrylate (104 mL, 1149 mmol) in MeOH (1.48 L) added dropwise at rt. The reaction was diluted with water (3 L) and extracted with EtOAc (3×5 L). The organic layer was dried (Na₂SO₄), concentrated under reduced pressure to give crude material that was purified by flash chromatography (0-50% EtOAc in hexane) to yield the title compound (180 g, 88.6%). LCMS (Method C): R_{T}=3.02 min, m/z=266 [M+H]⁺. ¹H NMR (400 MHz, CDCl₃): δ 7.31-7.23 (m, 5H), 4.07-4.04 (m, 1H), 3.64 (s, 3H), 3.62 (s, 3H), 2.69-2.55 (m, 4H), 2.49-2.37 (m, 2H).

Step 3: Methyl (S)-3-((tert-butoxycarbonyl)(3-methoxy-3-oxopropyl)amino)-3-phenylpropanoate: Methyl (S)-3-((3-methoxy-3-oxopropyl)amino)-3-phenylpropanoate (184 g, 694 mmol) was suspended in MeOH (1.84 L) and Boc₂O (191 mL, 833 mmol) was added dropwise. The reaction mixture was stirred at rt for 32 h. The reaction mixture was diluted with water (3 L) and extracted with ethyl acetate (3×5 L). The organic layer was dried (Na₂SO₄) and concentrated under reduced pressure. The residue was purified by flash chromatography (0-20% EtOAc in hexane) to yield title compound (220 g, 87%). LCMS (Method D): R_{T}=3.44 min, m/z=366 [M+H]⁺. ¹H NMR (400 MHz, CDCl₃): δ 7.33-7.24 (m, 5H), 5.69-5.52 (m, 1H), 3.65 (s, 3H), 3.58 (s, 3H), 3.32 (br s, 2H), 3.03-2.97 (m, 2H), 2.47 (br s, 1H), 2.16-2.02 (m, 1H), 1.45 (s, 9H).

Step 4: 1-tert-Butyl 3-methyl (6S)-4-hydroxy-6-phenyl-1,2,5,6-tetrahydropyridine-1,3-dicarboxylate and 1-tert-butyl 3-methyl (2S)-4-hydroxy-2-phenyl-1,2,3,6-tetrahydropyridine-1,3-dicarboxylate: Methyl (S)-3-((tert-butoxycarbonyl)(3-methoxy-3-oxopropyl)amino)-3-phenylpropanoate (55 g, 151 mmol) was dissolved in toluene (1.1 L) and cooled to −78° C. M solution of LiHMDS in THF (181 mL, 180 mmol) was added dropwise. After complete addition, the reaction mixture was stirred at −78° C. for 2 h. The reaction was quenched with water (1 L) and extracted using ethyl acetate (3×2 L). The organic layer was dried over Na₂SO₄ and concentrated under reduced pressure to give the crude material that was purified by flash chromatography (0-10% EtOAc in hexane) to yield the mixture of title compounds (25 g, 50%). LCMS (Method D): R_{T}=3.40, 3.69 min (2 compounds), m/z=334 [M+H]⁺.

Step 5: tert-Butyl (S)-4-oxo-2-phenylpiperidine-1-carboxylate: The mixture of 1-tert-butyl 3-methyl (6S)-4-hydroxy-6-phenyl-1,2,5,6-tetrahydropyridine-1,3-dicarboxylate and 1-tert-butyl 3-methyl (2S)-4-hydroxy-2-phenyl-1,2,3,6-tetrahydropyridine-1,3-dicarboxylate (40 g, 120 mmol) was dissolved in DMSO (200 mL). NaCl (21 g, 360 mmol) and water (7.5 mL) were added and the reaction mixture was heated at 145° C. for 6 h. The reaction mixture was diluted with water (500 mL) and extracted with ethyl acetate (2×2 L). The organic layer was dried (Na₂SO₄) and concentrated under reduced pressure. The crude compound was purified by flash chromatography (0-20% EtOAc in hexane) to yield title compound (20 g, 60%). LCMS (Method C): R_{T}=1.59 min, m/z=276 [M+H]⁺. ¹H NMR (400 MHz, CDCl₃): δ 7.33-7.22 (m, 5H), 5.70 (br s, 1H), 4.19 (br s, 1H), 3.18-3.11 (m, 1H), 2.97-2.92 (dd, 1H), 2.85-2.80 (dd, 1H), 2.53-2.47 (m, 1H), 2.37-2.31 (m, 1H), 1.46 (s, 9H).

Step 6: (S)-2-Phenylpiperidin-4-one hydrochloride: To a solution of tert-butyl (S)-4-oxo-2-phenylpiperidine-1-carboxylate (5 g, 18.2 mmol) in DCM (4 mL) was added 4 M HCl in 1,4-dioxane (20 mL) at 0° C. and the reaction mixture was stirred at rt for 16 h. The reaction was concentrated under reduced pressured to give crude title compound (3.18 g, 82%) that was used in next step without purification. LCMS (Method C): R_{T}=2.13 min, m/z=176 [M+H]⁺.

Step 7: (S)-2-Phenyl-1-(2,2,2-trifluoroacetyl)piperidin-4-one: (S)-2-Phenylpiperidin-4-one hydrochoride (3.8 g, 18.1 mmol) was suspended in DCM (100 mL). Et₃N (5.54 mL, 39.7 mmol) and trifluoroacetic anhydride (5.52 mL, 39.7 mmol) were added to the reaction mixture at 0° C. and stirred at rt for 16 h. The reaction mixture was diluted with water (100 mL) and extracted using ethyl acetate (2×250 mL). The organic layer was dried (Na₂SO₄) and concentrated under reduced pressure to give crude material that was purified by flash chromatography (0-30% EtOAc in hexane) to yield title compound (3.5 g, 71%). GCMS (Method A): m/z=271 M⁺.

Step 8: 2,2,2-Trifluoro-1-((2S)-4-((4-methoxybenzyl)amino)-2-phenylpiperidin-1-yl)ethan-1-one: To a solution of (S)-2-phenyl-1-(2,2,2-trifluoroacetyl)piperidin-4-one (1.0 g, 3.63 mmol) in MeOH (10 mL) were added 4-methoxybenzylamine (1.42 g, 10.9 mmol) and catalytic AcOH (1-2 drops) at rt. The reaction mixture was stirred for 1 h. NaBH₃CN (0.69 g, 10.9 mmol) was added to the reaction mixture and stirred for at rt for 16 h. The reaction mixture was diluted with water (50 mL) and extracted using ethyl acetate (2×100 mL). The organic layer was dried (Na₂SO₄) and concentrated under reduced pressure to give crude material that was purified by flash chromatography (0-10% MeOH in DCM) to yield title compound (750 mg, 52%). LCMS (Method D): R_{T}=3.45 min, m/z=393 [M+H]⁺.

Step 9: 1-((2S)-4-Amino-2-phenylpiperidin-1-yl)-2,2,2-trifluoroethan-1-one: To a solution of 2,2,2-trifluoro-1-((2S)-4-((4-methoxybenzyl)amino)-2-phenylpiperidin-1-yl)ethan-1-one (1.9 g, 4.85 mmol) in 1:1 MeCN/water (20 mL) was added CAN (7.97 g, 14.5 mmol) and the reaction mixture was stirred at rt for 5 h. The reaction mixture was quenched with saturated NaHCO_{3(aq)} solution (25 mL), diluted with water (50 mL), and extracted using ethyl acetate (3×100 mL). The combined organic phase was dried (Na₂SO₄) and concentrated under reduced pressure to give crude material that was purified by flash chromatography (0-5% MeOH in DCM) to yield title compound (950 mg, 72%). LCMS (Method C): R_{T}=2.54 min, m/z=273 [M+H]⁺.

Step 10: tert-Butyl ((2S,4R)-2-phenyl-1-(2,2,2-trifluoroacetyl)piperidin-4-yl)carbamate: To a solution of 1-((2S)-4-amino-2-phenylpiperidin-1-yl)-2,2,2-trifluoroethan-1-one (950 mg, 3.5 mmol) in DCM (5 mL) were added Et₃N (1.46 mL, 10.5 mmol) and Boc₂O (0.96 mL, 4.19 mmol) at 0° C. After addition, the reaction mixture was stirred at rt for 16 h. The reaction mixture was diluted with water (50 mL) and extracted using ethyl acetate (100 mL). The organic layer was washed with saturated NaHCO_{3(aq)} solution (50 mL), water (50 mL) and dried (Na₂SO₄). The solvent was evaporated under reduced pressure to give crude tert-butyl ((2S)-2-phenyl-1-(2,2,2-trifluoroacetyl)piperidin-4-yl)carbamate (mixture of diastereoisomers) that was purified and separated by flash chromatography (0-30% EtOAc in hexane) to give tert-butyl ((2S,4S)-2-phenyl-1-(2,2,2-trifluoroacetyl)piperidin-4-yl)carbamate) (first eluting diastereoisomer: 400 mg, 30%). LCMS (Method C): R_{T}=3.65 min, m/z=273 [M−Boc+H]⁺. [α]_{D}²⁵=+5.2 (c 0.25 in MeOH); and the title compound (second eluting diastereoisomer: 300 mg, 23%). LCMS (Method C): R_{T}=3.58 min, m/z=373 [M+H]⁺. [α]_{D}²⁵=+36.1 (c 0.25 in MeOH). [Note: stereochemistry of the title compound was assigned on the basis of comparison of the optical rotation data with that recorded for the same compound that was prepared via an alternative method outlined in WO2020115501, p 317: [α]_{D}²¹=+49.82 (c 0.25 in MeOH), previously unpublished].

Step 11: tert-Butyl ((2S,4R)-2-phenylpiperidin-4-yl)carbamate: tert-Butyl ((2S,4R)-2-phenyl-1-(2,2,2-trifluoroacetyl)piperidin-4-yl)carbamate (300 mg, 0.8 mmol) in 4:1 MeOH/H₂O (10 mL) was added K₂CO₃ (168 mg, 0.2 mmol) and stirred at rt for 16 h. The reaction mixture was diluted with water (25 mL) and extracted with ethyl acetate (2×50 mL). The organic was dried (Na₂SO₄) and concentrated under reduced pressure to give the crude material that was purified by flash chromatography (0-30% EtOAc in hexane) to yield title compound (150 mg, 67%, de=100%). LCMS (Method E): R_{T}=1.34 min, m/z=277 [M+H]⁺. ¹H NMR (400 MHz, DMSO-d₆): δ 7.34-7.19 (m, 5H), 6.79 (d, 1H), 3.54 (d, 1H), 3.40 (br s, 1H), 3.03 (d, 1H), 2.65-2.59 (m, 1H), 1.82-1.69 (m, 2H), 1.36 (s, 9H), 1.30-1.05 (m, 3H). [α]_{D}²⁰=+36.11 (c 0.25 in MeOH).

### Intermediate 2: tert-Butyl (R)-3-(3,5-difluorophenyl)piperazine-1-carboxylate

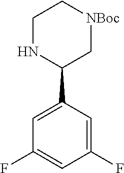 [SMILES: CC(C)(C)OC(=O)N1CCN[C@H](c2cc(F)cc(F)c2)C1]

tert-Butyl 3-(3,5-difluorophenyl)piperazine-1-carboxylate (10.2 g) [commercially available] was resolved into the single stereoisomers by chiral SFC using a Chiralpak IG (20 mm×250 mm, 5 μm) column with isocratic solvent conditions: 20:80 MeOH/CO₂ (0.2% v/v NH₃). The first eluted material afforded tert-butyl (S)-3-(3,5-difluorophenyl)piperazine-1-carboxylate (4.75 g). Chiral purity (Method A): R_{T}=1.07 min, 99.9% ee. The second eluted material afforded the title compound (4.83 g). Chiral purity (Method A): R_{T}=1.55 min, 99.7% ee. [Note: stereochemistry was assigned based on relative potencies of derivatives—the (R)-stereoisomers are known to be significantly more potent, see WO2020115501].

### Intermediate 3: tert-Butyl (R)-3-(2,5-difluorophenyl)piperazine-1-carboxylate

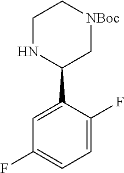 [SMILES: CC(C)(C)OC(=O)N1CCN[C@H](c2cc(F)ccc2F)C1]

tert-Butyl 3-(2,5-difluorophenyl)piperazine-1-carboxylate (9.88 g) [commercially available] was resolved into the single stereoisomers by chiral HPLC using a Lux A2 (21.2 mm×250 mm, 5 μm) column with isocratic solvent conditions: 50:50 heptane/IPA (0.2% v/v NH₃). The first eluted material afforded the title compound (4.55 g). Chiral purity (Method B): R_{T}=4.05 min, 100% ee. The second eluted material afforded tert-butyl (S)-3-(2,5-difluorophenyl)piperazine-1-carboxylate (4.47 g). Chiral purity (Method B): R_{T}=6.29 min, 100% ee. [Note: stereochemistry was assigned based on relative potencies of derivatives—the (R)-stereoisomers are known to be significantly more potent, see WO2020115501].

### Intermediate 4: (2S,4R)-4-((tert-Butyldimethylsilyl)oxy)-2-phenylpiperidine

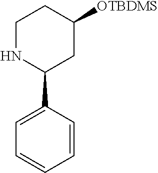 [SMILES: CC(C)(C)[Si](C)(C)O[C@@H]1CCN[C@H](c2ccccc2)C1]

Step 1: tert-Butyl (2S)-4-hydroxy-2-phenylpiperidine-1-carboxylate: To a solution of tert-butyl (S)-4-oxo-2-phenylpiperidine-1-carboxylate (2.5 g, 9.08 mmol) in THF (100 mL) was added 1 M LiAlH₄ in THF solution (10.9 mL, 10.9 mmol) at 0° C. under nitrogen and the reaction mixture was stirred at 0° C. for 40 min. The reaction mixture was quenched by dropwise addition of Na₂SO_{4(aq)} solution and diluted with water (50 mL) and ethyl acetate (100 mL). The organic layer was separated and washed with brine (75 mL) and dried (Na₂SO₄). The solvents were evaporated under reduced pressure to give the crude material that was purified by flash chromatography (0-20% EtOAc in hexane) to afford title compound (1.85 g, 73%). LCMS (Method D): R_{T}=3.07 min, m/z=278 [M+H]⁺.

Step 2: (2S,4R)-2-Phenylpiperidin-4-ol: To a solution of tert-butyl (2S)-4-hydroxy-2-phenylpiperidine-1-carboxylate (1.3 g, 4.69 mmol) in DCM (58.5 mL) was added TFA (6.5 mL) and the reaction mixture was stirred at rt for 4 h. The solvent was evaporated under reduced pressure. The residue was diluted with water (50 mL) and basified with NaHCO₃ solution up to ˜pH 9-10, extracted with ethyl acetate (2×100 mL). The organic layer was washed with brine (80 mL), dried (Na₂SO₄) and the solvents were evaporated under reduced pressure to give (2S)-2-phenylpiperidin-4-ol (690 mg, 83%). This material was resolved into the single stereoisomers by chiral HPLC using a Chiralpak IG (21 mm×250 mm, 5 μm) column with isocratic solvent conditions: 90:10 hexane/EtOH (0.1% v/v isopropylamine) to give the title compound (150 mg). GCMS (Method A): m/z=177 M+. ¹H NMR (400 MHz, DMSO-d₆): δ 7.36-7.29 (m, 4H), 7.26-7.23 (m, 1H, overlapping CDCl₃ signal), 3.82-3.74 (m, 1H), 3.64-3.61 (m, 1H), 3.25-3.20 (m, 1H), 2.82-2.75 (m, 1H), 2.15-2.11 (m, 1H), 2.03-1.99 (m, 1H), 1.54-1.44 (m, 2H) [Note: conformed with Literature data from Tetrahedron: Asymmetry, 1999, 10, 4231-4237 to confirm structural assignment].

Step 3: (2S,4R)-4-((tert-Butyldimethylsilyl)oxy)-2-phenylpiperidine: To a stirring solution of (2S,4R)-2-phenylpiperidin-4-ol (60 mg, 0.339 mmol) and TBDMSCI (128 mg, 0.846 mmol) in DCM (2 mL) was added imidazole (92 mg, 1.35 mmol) and the resulting mixture was stirred at rt for 18 h. The reaction was quenched with saturated NaHCO_{3(aq)} and extracted with DCM (×3) using a phase separator. The combined organic phases were concentrated in vacuo and the crude material was purified by flash chromatography using a Biotage Sfar Amino D column (0-50% DCM in cyclohexane) to give the title compound (77 mg, 78%). LCMS (Method B): R_{T}=1.04 min, m/z=292 [M+H]⁺.

### Intermediate 5: tert-Butyl ethyl((2S4R)-2-phenylpiperidin-4-yl)carbamate

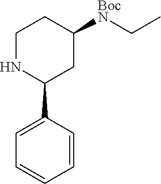 [SMILES: CCN(C(=O)OC(C)(C)C)[C@@H]1CCN[C@H](c2ccccc2)C1]

Step 1: 1-((2S)-4-(Ethylamino)-2-phenylpiperidin-1-yl)-2,2,2-trifluoroethan-1-one: To a solution of (S)-2-phenyl-1-(2,2,2-trifluoroacetyl)piperidin-4-one (1.6 g, 5.81 mmol) in MeOH (5 mL) were added 2 M solution of EtNH₂ in EtOH (8.22 mL, 17.4 mmol) and catalytic AcOH (1-2 drops) at rt and stirred for 1 h. NaBH₃CN (1.09 g, 17.4 mmol) was added to the reaction mixture and stirred at rt for 16 h. The reaction mixture was diluted with water (50 mL) and extracted with ethyl acetate (2×150 mL). The organic layer was dried (Na₂SO₄) and concentrated under reduced pressure to give crude material that was purified by flash chromatography (0-10% MeOH/DCM) to yield title compound (1.0 g, 57%). LCMS (Method D): R_{T}=2.61 min, m/z=301 [M+H]⁺.

Step 2: tert-Butyl ethyl((2S)-2-phenyl-1-(2,2,2-trifluoroacetyl)piperidin-4-yl)carbamate: To a solution of 1-((2S)-4-(ethylamino)-2-phenylpiperidin-1-yl)-2,2,2-trifluoroethan-1-one (1 g, 3.33 mmol) in DCM (10 mL) were added Et₃N (1.4 mL, 10 mmol) and Boc₂O (0.920 mL, 4 mmol) at 0° C. The reaction mixture was stirred at rt for 16 h. The reaction mixture was diluted with water (50 mL) and extracted with ethyl acetate (2×100 mL). The organic layer was washed with saturated NaHCO_{3(aq)} solution (80 mL), water (80 mL) and dried (Na₂SO₄). The solvent was evaporated under reduced pressure to give crude material that was purified by flash chromatography (0-20% EtOAc in hexane) to yield title compound (1 g, 75%). LCMS (Method D): R_{T}=3.81, 3.89 min, m/z=401 [M+H]⁺.

Step 3: tert-Butyl ethyl((2S,4R)-2-phenylpiperidin-4-yl)carbamate: To a solution of tert-butyl ethyl((2S)-2-phenyl-1-(2,2,2-trifluoroacetyl)piperidin-4-yl)carbamate (1 g, 2.5 mmol) in 4:1 MeOH/H₂O (10 mL) was added K₂CO₃ (518 mg, 3.75 mmol) at rt and stirred for 16 h. The reaction mixture was diluted with water (50 mL) and extracted with ethyl acetate (2×100 mL). The organic layer was dried (Na₂SO₄) and concentrated under reduced pressure to give crude material that was purified by flash chromatography (0-30% EtOAc in hexane) to yield tert-butyl ethyl((2S)-2-phenylpiperidin-4-yl)carbamate (600 mg, 79%). This material was resolved into the single stereoisomers by chiral HPLC using a Chiralpak AY-H (21 mm×250 mm, 5 μm) column with isocratic solvent conditions: 90:10 hexane/EtOH containing 0.1% v/v isopropylamine to give:

tert-butyl ethyl((2S,4S)-2-phenylpiperidin-4-yl)carbamate (first eluting: 130 mg, de=100%). LCMS (Method D): R_{T}=3.07 min, m/z=305 [M+H]⁺. ¹H NMR (400 MHz, CDCl₃): δ 7.48 (d, 2H), 7.35 (t, 2H), 7.23-7.20 (m, 1H), 4.34 (d, 1H), 4.11-4.08 (m, 1H), 3.26-3.12 (m, 2H), 2.90-2.85 (m, 1H), 2.75-2.69 (m, 1H), 2.33-2.29 (m, 1H), 2.07-1.99 (m, 1H), 1.73-1.70 (m, 1H), 1.68 (1H), 1.44 (s, 9H), 1.12 9t, 3H); and

the title compound (second eluting: 330 mg, de=100%). LCMS (Method D): R_{T}=3.14 min, m/z=305 [M+H]⁺. ¹H NMR (400 MHz, CDCl₃): δ 7.36-7.29 (m, 4H), 7.23-7.22 (m, 1H), 4.22 (bs, 1H), 3.68 (d, 1H), 3.27-3.22 (m, 1H), 3.14 (br s, 2H), 2.88-2.82 (m, 1H), 1.88-1.85 (m, 1H), 1.75-1.72 (m, 2H), 1.63 (br s, 1H), 1.45 (s, 9H), 1.07 (t, 3H).

[Note: the stereoisomers were confirmed by comparison of ¹H NMR nOe data].

### Intermediate 6: 2,2,2-Trifluoro-N-methyl-N-((2S,4R)-2-phenylpiperidin-4-yl)acetamide hydrochloride

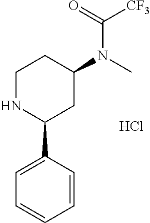 [SMILES: CN(C(=O)C(F)(F)F)[C@@H]1CCN[C@H](c2ccccc2)C1 ; Cl]

Step 1: tert-Butyl (2S)-4-(methylamino)-2-phenylpiperidine-1-carboxylate: To a solution of tert-butyl (S)-4-oxo-2-phenylpiperidine-1-carboxylate (5.00 g, 18.2 mmol) in MeOH (20 mL) were added 40% MeNH₂ in MeOH (1.69 g, 54.5 mmol) and catalytic AcOH (1-2 drops) at rt and stirred for 1 h. NaBH₃CN (3.44 g, 54.5 mmol) was added to the reaction mixture and stirred at rt for 16 h. The reaction mixture was diluted with water (100 mL) and extracted with ethyl acetate (2×250 mL). The organic layer was dried (Na₂SO₄) and concentrated under reduced pressure to give the crude material that was purified by flash chromatography (0-10% MeOH in DCM) to yield title compound (3.3 g, 62%). LCMS (Method C): R_{T}=2.77 min, m/z=291 [M+H]⁺.

Step 2: tert-Butyl (2S)-2-phenyl-4-(2,2,2-trifluoro-N-methylacetamido)piperidine-1-carboxylate: To a solution of tert-butyl (2S)-4-(methylamino)-2-phenylpiperidine-1-carboxylate (3.3 g, 11.4 mmol) in DCM (30 mL) was added Et₃N (3.5 mL, 25.0 mmol) followed by trifluoroacetic anhydride (3.48 mL, 25.0 mmol) at 0° C. The reaction mixture was stirred at rt for 16 h. The reaction mixture was diluted with water (50 mL) and extracted with ethyl acetate (2×100 mL). The organic layer was dried (Na₂SO₄) and concentrated under reduced pressure to give crude material that was purified by flash chromatography (0-30% EtOAc in hexane) to yield the title compound (2.5 g, 57%).

Step 3: tert-Butyl (2S,4R)-2-phenyl-4-(2,2,2-trifluoro-N-methylacetamido)piperidine-1-carboxylate: tert-Butyl (2S)-2-phenyl-4-(2,2,2-trifluoro-N-methylacetamido)piperidine-1-carboxylate (3.2 g) was separated into the single stereoisomers by chiral HPLC using a Chiralpak IC (20 mm×250 mm, 5 μm) column with isocratic solvent conditions: 95:5 hexane/EtOH to give:

tert-Butyl (2S,4S)-2-phenyl-4-(2,2,2-trifluoro-N-methylacetamido)piperidine-1-carboxylate (first eluting isomer, 0.8 g). LCMS (Method C): R_{T}=3.81 min, m/z=387 [M+H]⁺. ¹H NMR at 100° C. (400 MHz, DMSO-d₆): δ 7.39 (t, 2H), 7.29-7.25 (m, 3H), 5.53 (br s, 1H), 4.28-4.10 (m, 3H), 2.97-2.92 (m, 2H), 2.87-2.81 (m, 1H), 2.39-2.35 (m, 1H), 2.16 (br s, 1H), 1.82 (br s, 1H), 1.63-1.60 (m, 1H), 1.45 (s, 9H); and

the title compound (second eluting isomer: 1.2 g). LCMS (Method C): R_{T}=3.77 min, m/z=387 [M+H]⁺. ¹H NMR at 100° C. (400 MHz, DMSO-d₆): δ 7.34-7.21 (m, 5H), 4.85-4.81 (m, 1H), 4.40 (br s, 1H), 3.97-3.91 (m, 1H), 3.53-3.46 (m, 1H), 2.15-2.10 (m, 3H), 1.79 (br s, 1H), 1.41 (s, 9H).

[Note: the stereoisomers were confirmed by comparison of ¹H NMR nOe data].

Step 4: 2,2,2-Trifluoro-N-methyl-N-((2S,4R)-2-phenylpiperidin-4-yl)acetamide hydrochloride: tert-Butyl (2S,4R)-2-phenyl-4-(2,2,2-trifluoro-N-methylacetamido)piperidine-1-carboxylate (1.20 g, 3.11 mmol) was suspended in DCM (3 mL) followed by addition of 4 M HCl in 1,4-dioxane (10 mL). The reaction mixture was stirred at rt for 3 h. The solvent was evaporated under reduced pressure and residue was triturated with DCM and pentane to yield the title compound (890 mg, 89%, de=100%). LCMS (Method C): R_{T}=2.39 min, m/z=287 [M+H]⁺.

¹H NMR at 100° C. (400 MHz, DMSO-d₆): δ 9.39 (br s, 2H), 7.64 (d, 2H), 7.46-7.39 (m, 3H), 4.61 (br s, 1H), 4.45 (d, 1H), 3.40-3.44 (m, 1H), 3.27-3.21 (m, 1H), 3.0 (3H, overlapping with HDO signal), 2.44 (m, 1H), 1.99 (d, 1H), 1.87 (d, 1H).

### Intermediate 7: 3-(((2S,4R)-2-Phenylpiperidin-4-yl)amino)thietane 1,1-dioxide hydrochloride

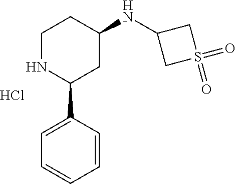 [SMILES: Cl ; O=S1(=O)CC(N[C@@H]2CCN[C@H](c3ccccc3)C2)C1]

Step 1: tert-Butyl (2S)-4-((1,1-dioxidothietan-3-yl)amino)-2-phenylpiperidine-1-carboxylate: To a solution of tert-butyl (S)-4-oxo-2-phenylpiperidine-1-carboxylate (500 mg, 1.82 mmol) in MeOH (10 mL) were added 3-aminothietane 1,1-dioxide (264 mg, 2.18 mmol) and catalytic AcOH (1 drop) at rt. The reaction mixture was stirred for 1 h. NaBH₃CN (344 mg, 5.45 mmol) was added to the reaction mixture and stirred at rt for 16 h. The reaction mixture was diluted with water (20 mL) and extracted with ethyl acetate (2×50 mL). The organic layer was dried (Na₂SO₄) and concentrated under reduced pressure to give the crude material that was purified by flash chromatography (0-10% MeOH in DCM) to yield the title compound (500 mg, 72%) that was carried to the next step.

Step 2: tert-Butyl (2S,4R)-4-((1,1-dioxidothietan-3-yl)amino)-2-phenylpiperidine-1-carboxylate: tert-Butyl (2S)-4-((1,1-dioxidothietan-3-yl)amino)-2-phenylpiperidine-1-carboxylate (1.2 g) was separated into the single stereoisomers by chiral HPLC using a Chiralpak AY-H (21 mm×250 mm, 5 μm) column with isocratic solvent conditions: 80:20 hexane/EtOH containing 0.1% v/v isopropylamine to give:

tert-Butyl (2S,4S)-4-((1,1-dioxidothietan-3-yl)amino)-2-phenylpiperidine-1-carboxylate (first eluting diastereoisomer: 125 mg). LCMS (Method D): R_{T}=3.21 min, m/z=381 [M+H]⁺; and the title compound (second eluting diastereoisomer: 292 mg). LCMS (Method C): R_{T}=3.39 min. m/z=381 [M+H]⁺.

Step 3: 3-(((2S,4R)-2-Phenylpiperidin-4-yl)amino)thietane 1,1-dioxide hydrochloride: To a suspension of tert-butyl (2S,4R)-4-((1,1-dioxidothietan-3-yl)amino)-2-phenylpiperidine-1-carboxylate (700 mg, 1.84 mmol) in DCM (2 mL) was added 4 M HCl in 1,4-dioxane (10 mL) at 0° C. and stirred at rt for 4 h. The reaction mixture was concentrated under reduced pressure to give crude material that was triturated using DCM and pentane to yield the title compound (575 mg, 98%, de=100%). LCMS (Method G): R_{T}=2.83 min, m/z=281 [M+H]⁺. ¹H NMR (400 MHz, methanol-d₄): δ 7.57-7.47 (m, 5H), 4.65-4.60 (m, 2H), 4.53-4.50 (m, 4H), 3.74 (t, 1H), 3.66-3.63 (m, 1H), 3.35 (d, 1H), 2.57-2.54 (m, 2H), 2.32-2.11 (m, 2H). [Note: the stereoisomer was confirmed by comparison of ¹H NMR nOe data with that of Intermediate 8].

### Intermediate 8: 3-(((2S,4S)-2-Phenylpiperidin-4-yl)amino)thietane 1,1-dioxide hydrochloride

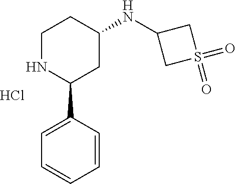 [SMILES: Cl ; O=S1(=O)CC(N[C@H]2CCN[C@H](c3ccccc3)C2)C1]

To a suspension of tert-butyl (2S,4S)-4-((1,1-dioxidothietan-3-yl)amino)-2-phenylpiperidine-1-carboxylate (300 mg, 0.79 mmol) in DCM (2 mL) was added 4 M HCl in 1,4-dioxane (7 mL) at 0° C. and stirred at rt for 4 h. The reaction mixture was concentrated under reduced pressure to give crude material that which was triturated using DCM and pentane to yield the title compound (245 mg, 98%, de=100%). LCMS (Method G): R_{T}=3.34 min, m/z=281 [M+H]⁺. ¹H NMR (400 MHz, methanol-d₄): δ 7.54-7.46 (m, 5H), 4.98 (d, 1H), 4.59-4.45 (m, 4H), 4.35-4.33 (m 1H), 3.80-3.71 (m, 2H), 3.44-3.41 (m, 1H), 2.49-2.36 (m, 2H), 2.29-2.27 m, 2H). [Note: the stereoisomer was confirmed by comparison of ¹H NMR nOe data with that of Intermediate 7].

### Intermediate 9: (2S,4R)-4-Methyl-2-phenylpiperidin-4-ol hydrochloride

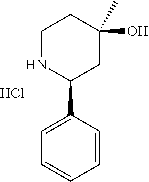

Step 1: (2S,4R)-4-Hydroxy-4-methyl-2-phenyl-piperidine-1-carboxylic acid tert-butyl ester: To a stirred solution of tert-butyl (S)-4-oxo-2-phenylpiperidine-1-carboxylate (1 g, 3.36 mmol) in THF (50 mL) was added dropwise a 3 M solution of MeMgBr in diethyl ether (2.42 mL, 7.26 mmol) at −10° C. and stirred at rt for 16 h. The reaction mixture was quenched by dropwise addition of saturated ammonium chloride_{(aq)} solution, diluted with water (50 mL) and extracted using ethyl acetate (3×50 mL). The organic layer was washed with brine (60 mL), dried (Na₂SO₄) and concentrated under reduced pressure to give the crude material that was purified by flash chromatography (0-75% EtOAc in hexane) to afford the title compound (0.5 g, 47%). LCMS (Method D): R_{T}=3.34 min, m/z=292 [M+H]⁺. ¹H NMR (400 MHz, DMSO-d₆): δ 7.29-7.25 (m, 2H), 7.19-7.14 (m, 3H), 5.11-5.10 (d, 1H), 3.21-3.12 (m, 1H), 2.22-2.28 (dd, 1H), 1.82-1.76 (dd, 1H), 1.47-1.44 (m, 2H), 1.32 (s, 9H), 1.13 (s, 3H).

Step 2: (2S,4R)-4-Methyl-2-phenylpiperidin-4-ol hydrochloride: (2S,4R)-4-Hydroxy-4-methyl-2-phenyl-piperidine-1-carboxylic acid tert-butyl ester (425 mg, 1.46 mmol) was dissolved in DCM (50 mL). 4 M HCl in 1,4-dioxane (12.5 mL) was added dropwise at 0° C. and stirred at rt for 4 h. The reaction mixture was concentrated under reduced pressure to give the crude material that was triturated with diethyl ether to yield the title compound (190 mg, 68%). LCMS (Method C): R_{T}=1.72 min, m/z=192 [M+H]⁺. ¹H NMR (400 MHz, DMSO-d₆): δ 9.27 (br s, 1H), 7.58-7.56 (d, 2H), 7.46-7.38 (m, 3H), 5.01 (s, 1H), 4.31-4.28 (d, 1H), 3.31-3.26 (m, 1H), 3.09-3.03 (m, 1H), 2.07-1.91 (m, 2H), 1.80-1.73 (m, 2H), 1.34 (s, 3H). [Note: Stereoisomer (only one formed) was confirmed by analysis of ¹H NMR nOe data].

### Intermediate 10: 2,2,2-Trifluoro-N-((2S,4R)-2-(3-fluorophenyl)piperidin-4-yl)-N-methylacetamide hydrochloride

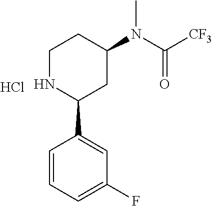 [SMILES: Cl ; CN(C(=O)C(F)(F)F)[C@@H]1CCN[C@H](c2cccc(F)c2)C1]

Step 1: tert-Butyl (S)-2-(3-fluorophenyl)-4-oxopiperidine-1-carboxylate: The title compound was prepared similarly to tert-butyl (S)-4-oxo-2-phenylpiperidine-1-carboxylate (Intermediate 1, Steps 1 to 5) except using (S)-3-amino-3-(3-fluorophenyl)propanoic acid instead of (S)-3-amino-3-phenylpropanoic acid as the starting material. LCMS (Method C): R_{T}=3.47 min, m/z=294 [M+H]⁺. ¹H NMR (400 MHz, CDCl₃): δ 7.31-7.27 (m, 1H), 57.01 (d, 1H), 6.97-6.93 (m, 2H), 5.66 (br s, 1H), 4.20 (br s, 1H), 3.18 (dt, 1H), 2.93-2.81 (m, 2H), 2.56-2.47 (m, 1H), 2.38-2.33 (m, 1H), 1.46 (s, 9H).

Step 2: tert-Butyl (2S)-2-(3-fluorophenyl)-4-(methylamino)piperidine-1-carboxylate: To a stirred solution of tert-butyl (S)-2-(3-fluorophenyl)-4-oxopiperidine-1-carboxylate (2.5 g, 8.52 mmol) in MeOH (100 mL) was added MeNH₂ (10 mL) and stirred at rt for 1 h. NaBH₃CN (2.4 g, 38.4 mmol) was added portionwise at rt. After 24 h, the reaction mixture was quenched using water (25 mL) and concentrated under reduced pressure to remove volatiles. The remaining mixture was diluted with water (50 mL) and extracted using ethyl acetate (2×100 mL). The combined organic phase was dried (Na₂SO₄) and concentrated to give the crude product that was purified by flash chromatography (70-80% EtOAc in hexane) to yield the title compound (2.5 g, 95%). LCMS (Method E): R_{T}=1.53 min, m/z=309 [M+H]⁺.

Step 3: tert-Butyl (2S)-2-(3-fluorophenyl)-4-(2,2,2-trifluoro-N-methylacetamido)piperidine-1-carboxylate: To a stirred solution of tert-butyl (2S)-2-(3-fluorophenyl)-4-(methylamino)piperidine-1-carboxylate (2.5 g, 8.11 mmol) in DCM (50 mL) was added triethylamine (4.5 mL, 32.4 mmol) followed by trifluoroacetic anhydride (2.5 mL 17.8 mmol) at 0° C. and the reaction mixture was stirred at rt. After 16 h, the reaction mixture was diluted with water (70 mL) and extracted with DCM (2×150 mL). The combined organic phase was dried (Na₂SO₄) and concentrated to give the crude product that was purified by flash chromatography (30% EtOAc in hexane) to yield the title compound (1.2 g, 40%).

Step 4: tert-Butyl (2S,4R)-2-(3-fluorophenyl)-4-(2,2,2-trifluoro-N-methylacetamido)piperidine-1-carboxylate: tert-Butyl (2S)-2-(3-fluorophenyl)-4-(2,2,2-trifluoro-N-methylacetamido)piperidine-1-carboxylate (1.2 g) was separated into the single stereoisomers by chiral HPLC using a Chiralpak IC (20 mm×250 mm, 5 μm) column with isocratic solvent conditions: 95:5 hexane/EtOH to give: tert-Butyl (2S,4S)-2-(3-fluorophenyl)-4-(2,2,2-trifluoro-N-methylacetamido)piperidine-1-carboxylate (first eluting isomer: 0.25 g, de=100%). LCMS (Method D): R_{T}=3.58 min, m/z=405 [M+H]⁺. ¹H NMR (400 MHz, CDCl₃): δ 7.33 (br s, 1H), 7.12-7.85 (m, 3H), 5.71-5.53 (br s, 2 peaks, 1H), 4.53-4.11 (m, 2H), 2.99-2.92 (s, 2 peaks, 3H), 2.85-2.71 (m, 1H), 2.35 (d, 1H), 2.21-2.00 (m, 1H), 1.78-1.60 (m, 2H), 1.52 (s, 2 peaks, 9H); and the title compound (second eluting isomer: 0.45 g, de=100%). LCMS (Method D): R_{T}=3.58 min, m/z=405 [M+H]⁺. ¹H NMR (400 MHz, CDCl₃): δ 7.28-7.23 (m, 1H), 6.98-6.88 (m, 3H), 4.79 (t, 1H), 4.77-4.58 (m, 1H), 4.12-4.06 (m, 1H), 3.51-3.45 (m, 1H), 2.97-2.88 (m, 3H), 2.21-2.15 (m, 1H), 2.07-1.97 (m, 2H), 1.71-1.69 (m, 1H), 1.25 (s, 9H).

[Note: the stereoisomers were confirmed by comparison of ¹H NMR nOe data].

Step 5: 2,2,2-Trifluoro-N-((2S,4R)-2-(3-fluorophenyl)piperidin-4-yl)-N-methylacetamide hydrochloride: To a stirred solution of tert-butyl (2S,4R)-2-(3-fluorophenyl)-4-(2,2,2-trifluoro-N-methylacetamido)piperidine-1-carboxylate (650 mg, 1.61 mmol) in DCM (10 mL) was added 4 M HCl in 1,4-dioxane (10 mL) at 0° C. The reaction mixture was stirred for 3 h at 0° C. before concentration under reduced pressure. The remaining crude product was triturated with diethyl ether and pentane to yield title compound (445 mg, 81%, de=100%). LCMS (Method C): R_{T}=2.71 min, m/z=305 [M+H]⁺. ¹H NMR at 100° C. (400 MHz, DMSO-d₆): δ 9.60 (br s, 2H), 7.58-7.43 (m, 3H), 7.28-7.24 (m, 1H), 4.67-4.52 (m, 2H), 3.46-3.43 (m, 1H), 3.24-3.21 (m, 1H), 3.01-2.92 (s, 2 peaks, 3H), 2.36-2.27 (m, 2H), 1.98-1.82 (m, 2H).

### Intermediate 11: 2,2,2-Trifluoro-N-((2S,4R)-2-(3-fluorophenyl)piperidin-4-yl)acetamide hydrochloride

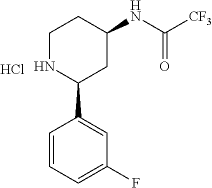 [SMILES: Cl ; O=C(N[C@@H]1CCN[C@H](c2cccc(F)c2)C1)C(F)(F)F]

Step 1: tert-Butyl (2S)-4-amino-2-(3-fluorophenyl)piperidine-1-carboxylate: To a stirred solution of tert-butyl (S)-2-(3-fluorophenyl)-4-oxopiperidine-1-carboxylate (6.0 g, 20.5 mmol) in MeOH (10 mL) was added 7 M NH₃ in MeOH (30 mL) followed by catalytic AcOH (1-2 drops) and stirred at rt for 1 h. NaBH₃CN (3.86 g, 61.4 mmol) was added portionwise at rt and allowed to stir at rt. After 16 h, the reaction mixture was diluted with water (50 mL) and extracted using ethyl acetate (2×150 mL). The combined organic phase was dried (Na₂SO₄) and concentrated to give crude material that was purified by flash chromatography (10% MeOH in DCM) to yield the title compound (2.6 g, 43%). LCMS (Method C): R_{T}=2.81 min, m/z=295 [M+H]⁺.

Step 2: tert-Butyl (2S)-2-(3-fluorophenyl)-4-(2,2,2-trifluoroacetamido)piperidine-1-carboxylate: To a stirred solution of tert-butyl (2S)-4-amino-2-(3-fluorophenyl)piperidine-1-carboxylate (2.4 g, 8.16 mmol) in DCM (25 mL) was added TEA (3.66 mL, 26.1 mmol) followed by trifluoroacetic anhydride (2.50 mL, 18.0 mmol) at 0° C. and the reaction mixture was stirred at rt. After 16 h, the reaction mixture was diluted with water (70 mL) and extracted using DCM (2×150 mL). The organic phase was dried (Na₂SO₄) and concentrated to give crude material that was purified by flash chromatography (30% EtOAc in hexane) to yield the title compound (2.0 g, 62%).

Step 3: tert-Butyl (2S,4R)-2-(3-fluorophenyl)-4-(2,2,2-trifluoroacetamido)piperidine-1-carboxylate: tert-Butyl (2S)-2-(3-fluorophenyl)-4-(2,2,2-trifluoroacetamido)piperidine-1-carboxylate (2.0 g) was separated into the single stereoisomers by chiral HPLC using a Chiralpak IC (20 mm×250 mm, 5 μm) column with isocratic solvent conditions: 95:5 hexane/EtOH to give:

tert-Butyl (2S,4S)-2-(3-fluorophenyl)-4-(2,2,2-trifluoro-N-methylacetamido)piperidine-1-carboxylate (first eluting isomer: 0.45 g, de=100%). LCMS (Method C): R_{T}=3.71 min, m/z=391 [M+H]⁺; and

the title compound (second eluting isomer: 0.65 g, de=100%). LCMS (Method C): R_{T}=3.63 min, m/z=391 [M+H]⁺.

[Note: the stereoisomers were confirmed by comparison of ¹H NMR nOe data].

Step 4: 2,2,2-Trifluoro-N-((2S,4R)-2-(3-fluorophenyl)piperidin-4-yl)acetamide hydrochloride: To a stirred solution of tert-butyl (2S,4R)-2-(3-fluorophenyl)-4-(2,2,2-trifluoroacetamido)piperidine-1-carboxylate (650 mg, 1.67 mmol) in DCM (5 mL) was added 4 M HCl in dioxane (10 mL) at 0° C. and the reaction mixture was stirred at rt for 3 h. The reaction mixture was concentrated under reduced pressure and lyophilized to yield title compound (480 mg, 99%). LCMS (Method C): R_{T}=2.87 min, m/z=291 [M+H]⁺. ¹H NMR at 100° C. (400 MHz, DMSO-d₆): δ 9.59 (br s, 2H), 9.37 (br s, 1H), 7.52-7.43 (m, 3H), 7.21 (t, 1H), 4.46-4.43 (m, 1H), 4.17-4.12 (m, 1H), 3.43 (d, 1H), 3.21-3.14 (m, 1H), 2.18-2.02 (m, 4H).

### Intermediate 12: (R)-1-((7-Azaspiro[4.5]decan-10-yl)methyl)-4-chloropyridin-2(1H)-one hydrochloride

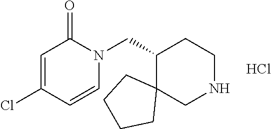 [SMILES: O=c1cc(Cl)ccn1C[C@@H]1CCNCC12CCCC2 ; Cl]

A solution of tert-butyl (R)-10-((4-chloro-2-oxopyridin-1(2H)-yl)methyl)-7-azaspiro[4.5]decane-7-carboxylate (300 mg, 0.788 mmol) [Example 44, Step 2] in 4 M HCl in 1,4-dioxane (2 mL) was stirred at rt for 18 h. The reaction was then concentrated in vacuo to give the title compound (249 mg, quant.). LCMS (Method B): R_{T}=0.62 min, m/z=281, 283 [M+H]⁺.

### Intermediate 13: 2-(Trimethylsilyl)ethyl ((2R,4R)-2-ethylpiperidin-4-yl)carbamate

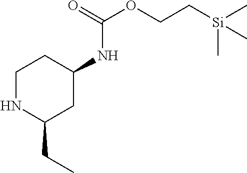 [SMILES: CC[C@@H]1C[C@H](NC(=O)OCC[Si](C)(C)C)CCN1]

Step 1: rac-(2R,4R)-2-Ethylpiperidine-4-carboxylic acid: To 2-ethylpyridine-4-carboxylic acid (5.00 g, 33 mmol) in methanol (150 mL) was added 10% (w/w) Pd/C (2.00 g) and stirred under hydrogen (100 atm) at 50° C. After 48 h, the reaction mixture was filtered and evaporated to dryness to give the title compound (4.72 g, 90%). ¹H NMR (400 MHz, DMSO-d₆): δ 9.21 (br s, 1H), 3.22 (m, 1H), 2.93 (m, 1H), 2.84 (m, 1H), 2.53 (m, 1H), 2.03 (m, 1H), 1.93 (m, 1H), 1.69 (m, 2H), 1.2 (m, 1H), 1.40 (m, 1H), 0.90 (t, 3H).

Step 2: rac-Benzyl (2R,4R)-2-ethyl-4-(((2-(trimethylsilyl)ethoxy)carbonyl)amino)piperidine-1-carboxylate: To rac-(2R,4R)-2-ethylpiperidine-4-carboxylic acid (4.72 g, 30.0 mmol) in 1,4-dioxane (200 mL) and water (100 mL) was added sodium hydroxide (4.80 g, 120 mmol). The reaction mixture was cooled to 0° C. and CbzCl (7.70 g, 45.0 mmol) was added dropwise. After 24 h, the volatiles were evaporated and the remaining aqueous phase washed with MTBE, acidified to ˜pH 4 using saturated sodium hydrogensulfate_{(aq)} solution which gave an oil that was extracted using EtOAc. Evaporation of the solvents gave crude rac-(2R,4R)-1-((benzyloxy)carbonyl)-2-ethylpiperidine-4-carboxylic acid (7.8 g) which was dissolved in toluene (200 mL), followed by addition of diphenylphosphoryl azide (11.1 g, 40.0 mmol) and triethylamine (4.06 g, 40.0 mmol). The reaction mixture was heated at 75° C. for 4 h, followed by the addition of trimethylsilylethanol (9.50 g, 80.0 mmol). The reaction mixture was heated at 100° C. After 24 h, the solvents were removed in vacuo and the remaining residue was purified by flash chromatography to give the title compound (5.40 g, 44%). ¹H NMR (400 MHz, DMSO-d₆): δ 7.30 (m, 5H), 7.09 (br s, 1H), 5.02 (m, 2H), 4.00 (m, 2H), 3.81 (m, 1H), 3.64 (m, 1H), 3.53 (m, 1H), 3.22 (m, 1H), 1.71-1.63 (m, 4H), 1.47 (m, 2H), 0.88 (m, 2H), 0.71 (t, 3H), −0.02 (s, 9H).

Step 3: rac-2-(Trimethylsilyl)ethyl ((2R,4R)-2-ethylpiperidin-4-yl)carbamate: rac-Benzyl (2R,4R)-2-ethyl-4-(((2-(trimethylsilyl)ethoxy)carbonyl)amino)piperidine-1-carboxylate (5.40 g, 13.3 mmol) was dissolved in methanol (70 mL) and 10% (w/w) Pd/C (0.5 g) was added and stirred under hydrogen (1 atm) at rt. After 24 h, the reaction mixture was filtered, the solvents were removed in vacuo and the residue was purified by flash chromatography to give the title compound (1.72 g, 47%). LCMS (Method F): R_{T}=0.92 min, m/z=273 [M+H]⁺. ¹H NMR (400 MHz, DMSO-d₆): δ 7.01 (m, 1H), 4.00 (m, 2H), 3.25 (m, 1H), 2.91 (m, 1H), 2.27 (m, 1H), 1.75-1.61 (m, 2H), 1.30-1.11 (m, 3H), 0.88 (m, 2H), 0.83 (t, 3H), −0.01 (s, 9H).

Step 4: 2-(Trimethylsilyl)ethyl ((2R,4R)-2-ethylpiperidin-4-yl)carbamate: rac-2-(Trimethylsilyl)ethyl ((2R,4R)-2-ethylpiperidin-4-yl)carbamate (1.4 g) was resolved into the single stereoisomers by chiral HPLC using a Chiralpak AD-H (20 mm×250 mm, 5 μm) column with isocratic solvent conditions: 80:10:10 hexane/IPA/MeOH. The first eluted material (R_{T}=9.48 min) afforded 2-(trimethylsilyl)ethyl ((2S,4S)-2-ethylpiperidin-4-yl)carbamate (0.65 g). [α]_{D}²⁵=−9.88 (c 0.25 in CHCl₃); and the second eluted material (R_{T}=13.27 min) afforded the title compound (0.62 g). [α]_{D}²⁵=+11.42 (c 0.25 in CHCl₃).

### Intermediate 14: tert-Butyl (R)-3,3-dimethyl-4-((2-oxo-4-chloropyridin-1(2H-yl)methyl)piperidine-1-carboxylate

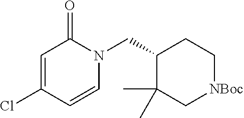 [SMILES: CC(C)(C)OC(=O)N1CC[C@@H](Cn2ccc(Cl)cc2=O)C(C)(C)C1]

Step 1: tert-Butyl 4-(iodomethyl)-3,3-dimethylpiperidine-1-carboxylate: To a stirred mixture of tert-butyl 4-(hydroxymethyl)-3,3-dimethyl-piperidine-1-carboxylate (15.3 g, 63.0 mmol), imidazole (5.14 g, 75.6 mmol) and triphenylphosphine (19.8 g, 75.6 mmol) in THF (153 mL), a solution of iodine (19.2 g, 75.6 mmol) in THF (150 mL) was added dropwise at 0° C. The reaction mixture was stirred overnight at rt, quenched by saturated Na₂SO_{3(aq)} solution until the colour completely disappeared, followed by extraction using ethyl acetate (3×500 mL). The combined organic phase was washed sequentially using brine (300 mL), water (300 mL), brine (300 mL), dried (Na₂SO₄) and the solvent was removed in vacuo. The remaining residue was purified by flash chromatography (10% MTBE in hexane) to give the title compound (18 g, 81%) as a viscous colorless oil. ¹H NMR (500 MHz, CDCl₃): δ 4.20 (s, 1H), 3.54 (m, 2H), 2.78 (t, 1H), 2.66 (s, 1H), 2.43 (s, 1H), 1.98 (m, 1H), 1.54 (m, 1H), 1.44 (s, 9H), 1.25 (m, 1H), 0.97 (s, 3H), 0.78 (s, 3H).

Step 2: tert-Butyl 3,3-dimethyl-4-((2-oxo-4-chloropyridin-1(2H)-yl)methyl)piperidine-1-carboxylate: A mixture of 4-chloropyridin-2(1H)-one (2.70 g, 20.8 mmol), tert-butyl 4-(iodomethyl)-3,3-dimethylpiperidine-1-carboxylate (8.83 g, 25 mmol), Cs₂CO₃ (8.15 g, 25 mmol) and 1,4-dioxane (106 mL) was placed into a sealed tube and heated at 120° C. for 48 h. After cooling to rt, the solvents were evaporated. The residue was dissolved in DCM, filtered, and the solvents were evaporated. The remaining residue was purified by flash chromatography to give the title compound (2.3 g, 26%). LCMS (Method F): R_{T}=1.34 min, m/z=299 [M−butene+H]⁺.

Step 3: tert-Butyl (R)-3,3-dimethyl-4-((2-oxo-4-chloropyridin-1(2H)-yl)methyl)piperidine-1-carboxylate: tert-Butyl 3,3-dimethyl-4-((2-oxo-4-chloropyridin-1(2H)-yl)methyl)piperidine-1-carboxylate (1.62 g) that was resolved into the single stereoisomers by chiral HPLC using a Chiralcel OD-H (4.6 mm×250 mm, 5 μm) column with isocratic solvent conditions: 80:10:10 hexane/IPA/MeOH. The first eluted material (R_{T}=7.79 min) afforded tert-butyl (S)-3,3-dimethyl-4-((2-oxo-4-phenylpyridin-1(2H)-yl)methyl)piperidine-1-carboxylate (674 mg). LCMS (Method F): R_{T}=1.30 min, m/z=299 [M−butene+H]⁺. ¹H NMR (400 MHz, DMSO-d₆): δ 7.72 (d, 1H), 6.51 (s, 1H), 6.35 (d, 1H), 4.12 (d, 1H), 3.90 (m, 1H), 3.50 (m, 2H), 2.60 (m, 1H), 2.42 (m, 1H), 1.68 (m, 1H), 1.38 (s, 9H), 1.27 (m, 1H), 0.97 (s, 3H), 0.81 (s, 3H). [α]_{D}²⁵=+85.58 (c 0.4 in MeOH); and the second eluted material (R_{T}=9.44 min) afforded the title compound (692 mg). LCMS (Method F): R_{T}=1.30 min, m/z=299 [M−butene+H]⁺. ¹H NMR (400 MHz, DMSO-d₆): δ 7.72 (d, 1H), 6.51 (s, 1H), 6.35 (d, 1H), 4.12 (d, 1H), 3.90 (m, 1H), 3.50 (m, 2H), 2.60 (m, 1H), 2.42 (m, 1H), 1.68 (m, 1H), 1.38 (s, 9H), 1.27 (m, 1H), 0.97 (s, 3H), 0.81 (s, 3H). [α]_{D}²⁵=−78.98 (c 0.4 in MeOH).

### Intermediate 15: tert-Butyl ((3S,5S)-5-phenylpyrrolidin-3-yl)carbamate

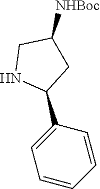 [SMILES: CC(C)(C)OC(=O)N[C@@H]1CN[C@H](c2ccccc2)C1]

Step 1: rac-tert-Butyl ((3S,5S)-5-phenyl-1-(2,2,2-trifluoroacetyl)pyrrolidin-3-yl)carbamate: To rac-tert-butyl ((3S,5S)-5-phenylpyrrolidin-3-yl)carbamate (2.52 g, 9.6 mmol) in DCM (100 mL) at 0° C. was added triethylamine (2.92 g, 28.8 mmol) followed by dropwise addition of trifluoroacetic anhydride (2.22 g, 10.6 mmol). After 24 h, the reaction mixture was washed several times with water, the combined organic phase was dried (Na₂SO₄) and the solvents were removed in vacuo to give the title compound (3.41 g, 99%). LCMS (Method F): R_{T}=1.23 min, m/z=303 [M−butene+H]⁺.

Step 2: tert-Butyl ((3S,5S)-5-phenyl-1-(2,2,2-trifluoroacetyl)pyrrolidin-3-yl)carbamate: rac-tert-Butyl ((3S,5S)-5-phenyl-1-(2,2,2-trifluoroacetyl)pyrrolidin-3-yl)carbamate (2.80 g) was resolved into the single stereoisomers by chiral HPLC using a Chiralpak IB (20 mm×250 mm, 5 μm) column with isocratic solvent conditions: 90:5:5 hexane/IPA/MeOH (Flow rate: 12 mL/min). The first eluted material (R_{T}=12.12 min) afforded the title compound (1.49 g). [α]_{D}²⁵=−9.76 (c 0.25 in CHCl₃); and the second eluted material (R_{T}=25.22 min) afforded tert-butyl ((3R,5R)-5-phenyl-1-(2,2,2-trifluoroacetyl)pyrrolidin-3-yl)carbamate (0.95 g). [α]_{D}²⁵=+10.44 (c 0.25 in CHCl₃).

Step 3: tert-Butyl ((3S,5S)-5-phenylpyrrolidin-3-yl)carbamate: To a stirred solution of tert-butyl ((3S,5S)-5-phenyl-1-(2,2,2-trifluoroacetyl)pyrrolidin-3-yl)carbamate (1.49 g, 4.16 mmol) in DCM (70 mL) was added 2 M K₂CO_{3(aq)} solution (20 mL) at rt. After 24 h, stirring was stopped and the resultant biphasic mixture was separated and extracted using further DCM (×3). The combined organic phase was dried (Na₂SO₄) and the solvents were removed in vacuo to give the title compound (0.89 g, 82%). LCMS (Method F): R_{T}=0.76 min, m/z=263 [M+H]⁺. ¹H NMR (500 MHz, CDCl₃): δ 7.38-7.24 (m, 5H), 4.83 (m, 1H), 4.24 (m, 1H), 4.14 (m, 1H), 3.24 (m, 1H), 3.01 (m, 1H), 2.64 (m, 1H), 2.22 (m, 1H), 1.51 (m, 1H), 1.44 (s, 9H). [α]_{D}²¹=−19.76 (c 0.25 in MeOH).

### Intermediate 16: (2S,4R)—N-(2,2-Difluoroethyl)-2-phenylpiperidin-4-amine hydrochloride

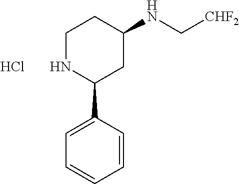 [SMILES: Cl ; FC(F)CN[C@@H]1CCN[C@H](c2ccccc2)C1]

Step 1: tert-Butyl (2S)-4-((2,2-difluoroethyl)amino)-2-phenylpiperidine-1-carboxylate: To a stirred solution of tert-butyl (S)-4-oxo-2-phenylpiperidine-1-carboxylate (1.0 g, 3.63 mmol) in MeOH (10 mL) were added 2,2-difluoroethan-1-amine (0.512 mL, 7.26 mmol) and catalytic AcOH (1-2 drops) at rt. After 1 h, NaBH₃CN (0.69 g, 10.9 mmol) was added. After 16 h, the reaction mixture was diluted with water (50 mL) and extracted with ethyl acetate (2×100 mL). The combined organic phase was dried (Na₂SO₄) and concentrated under reduced pressure to give crude material that was purified by flash chromatography (0-10% MeOH in DCM) to yield the title compound (1.0 g, 80%).

Step 2: tert-Butyl (2S,4R)-4-((2,2-difluoroethyl)amino)-2-phenylpiperidine-1-carboxylate: tert-Butyl (2S)-4-((2,2-difluoroethyl)amino)-2-phenylpiperidine-1-carboxylate (1.1 g) was separated into the single stereoisomers by chiral HPLC using a Chiralcel OD-H (20 mm×250 mm, 5 μm) column with isocratic solvent conditions: 95:5 hexane/EtOH+0.1% triethylamine to give tert-butyl (2S,4S)-4-((2,2-difluoroethyl)amino)-2-phenylpiperidine-1-carboxylate (first eluting isomer: 300 mg, de=100%). LCMS (Method D): R_{T}=3.48 min, m/z=341 [M+H]⁺; and the title compound (second eluting isomer: 600 mg, de=100%). LCMS (Method D): R_{T}=3.48 min, m/z=341 [M+H]⁺. [Note: the stereoisomers were confirmed by comparison of ¹H NMR nOe data].

Step 3: (2S,4R)—N-(2,2-Difluoroethyl)-2-phenylpiperidin-4-amine hydrochloride: To a solution of tert-butyl (2S,4R)-4-((2,2-difluoroethyl)amino)-2-phenylpiperidine-1-carboxylate (600 mg, 1.77 mmol) in DCM (5 mL) was added 4 M HCl in 1,4-dioxane solution (5 mL) and the reaction mixture was stirred at rt. After 16 h, the solvent was removed in vacuo and the remaining residue was triturated with pentane to yield title compound (412 mg, 97%). LCMS (Method C): R_{T}=1.82 min, m/z=241 [M+H]⁺. ¹H NMR (400 MHz, DMSO-d₆): δ 10.01 (br s, 2H), 9.82 (br s, 1H), 9.46 (br s, 1H), 7.64-7.58 (m, 2H), 7.48-7.42 (m, 3H), 6.47 (t, 1H), 4.37 (t, 1H), 3.56-3.40 (m, 4H), 3.16-3.12 (m, 1H), 2.49 (m, 1H, overlapping DMSO signal), 2.34-2.31 (m, 1H), 2.18-2.10 (m, 2H).

### Intermediate 17: Methyl (2S,4R)-2-phenylpiperidine-4-carboxylate

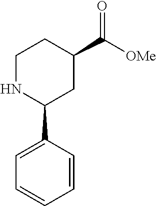 [SMILES: COC(=O)[C@@H]1CCN[C@H](c2ccccc2)C1]

Step 1: 2-Phenylisonicotinic acid: The title compound was prepared according to General Procedure 5 using methyl 2-bromoisonicotinate (10.8 g, 50 mmol) [commercially available], phenylboronic acid (9.15 g, 75 mmol), potassium phosphate tribasic (31.8 g, 150 mmol) and Pd(dppf)Cl₂·DCM (1.22 g, 1.5 mmol) in 3:1 1,4-dioxane/water (400 mL) to give the title compound (9.5 g, 95%). LCMS (Method F): R_{T}=1.01 min, m/z=200 [M+H]⁺.

Step 2: Methyl 2-phenylisonicotinate: To a solution of 2-phenylisonicotinic acid (9.5 g, 47.7 mmol) in methanol (285 mL) was added thionyl chloride (17.0 g, 143 mmol) dropwise at 0° C. The reaction mixture was refluxed and after 16 h, the solvents were removed in vacuo and the resultant material was dried under vacuum at 60° C. to yield the title compound (10.0 g, 98%). ¹H NMR (500 MHz, CDCl₃): δ 9.11 (d, 1H), 8.62 (s, 1H), 8.26 (m, 3H), 7.65 (m, 3H), 4.08 (s, 3H).

Step 3: rac-Methyl (2S,4R)-2-phenylpiperidine-4-carboxylate: To a solution of methyl 2-phenylisonicotinate (10.0 g, 47.0 mmol) [commercially available] in methanol (400 mL) was added 10% (w/w) Pd/C (2.0 g) and the reaction mixture was placed in an autoclave and hydrogenated (100 atm) at 60° C. After 48 h, the reaction mixture was filtered and evaporated to dryness to give the title compound (10 g, 97%). LCMS (Method F): R_{T}=0.60 min, m/z=220 [M+H]⁺. 1H NMR (500 MHz, DMSO-d₆): δ 9.55 (s, 1H), 7.58 (d, 2H), 7.41 (m, 3H), 4.29 (t, 1H), 3.61 (s, 3H), 3.37 (m, 1H), 3.09 (t, 1H), 2.88 (t, 1H), 2.11 (d, 1H), 2.00 (m, 3H).

Step 4: Methyl (2S,4R)-2-phenylpiperidine-4-carboxylate: rac-Methyl (2S,4R)-2-phenylpiperidine-4-carboxylate (0.80 g) was resolved into the single stereoisomers by chiral HPLC using a Chiralpak OD-H (4.6 mm×250 mm, 5 μm) column with isocratic solvent conditions: 80:10:10 hexane/IPA/MeOH (Flow rate: 0.6 mL/min). The first eluted material (R_{T}=10.93 min) afforded methyl (2R,4S)-2-phenylpiperidine-4-carboxylate (281 mg). [α]_{D}²⁵=−37.86 (c 0.4 in MeOH); and the second eluted material (R_{T}=12.87 min) afforded the title compound (275 mg). [α]_{D}²⁵=+36.57 (c 0.4 in MeOH).

### Intermediate 18: tert-Butyl (R)-10-((6-oxo-4-(o-tolyl)pyrimidin-1(6H)-yl)methyl)-7-azaspiro[4.5]decane-7-carboxylate

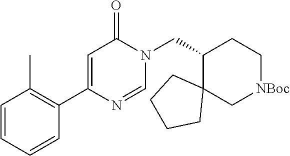 [SMILES: Cc1ccccc1-c1cc(=O)n(C[C@@H]2CCN(C(=O)OC(C)(C)C)CC23CCCC3)cn1]

Step 1: tert-Butyl 10-((6-oxo-4-(o-tolyl)pyrimidin-1(6H)-yl)methyl)-7-azaspiro[4.5]decane-7-carboxylate: To 6-(o-tolyl)pyrimidin-4(3H)-one (7.00 g, 37.6 mmol) [commercially available] in 1,4-dioxane (400 mL) was added tert-butyl 10-(iodomethyl)-7-azaspiro[4.5]decane-7-carboxylate (20.0 g, 52.6 mmol) and 2 eq. of cesium carbonate (24.5 g, 75.2 mmol). The reaction mixture was heated at 100° C. and stirred. After 40 h, the reaction mixture was filtered and the solvents were removed in vacuo. The remaining residue was purified by flash chromatography to give the title compound (6.40 g, 39%). LCMS (Method F): R_{T}=1.68 min, m/z=382 [M−butene+H]⁺.

Step 2: tert-Butyl (R)-10-((6-oxo-4-(o-tolyl)pyrimidin-1(6H)-yl)methyl)-7-azaspiro[4.5]decane-7-carboxylate: tert-Butyl 10-((6-oxo-4-(o-tolyl)pyrimidin-1 (6H)-yl)methyl)-7-azaspiro[4.5]decane-7-carboxylate (7.30 g) was resolved into the single stereoisomers by chiral HPLC using a Chiralpak IA-Ill (20 mm×250 mm, 5 μm) column with isocratic solvent conditions: 90:5:5 hexane/IPA/MeOH (Flow rate: 15 mL/min). The first eluted material (R_{T}=17.25 min) afforded tert-butyl (S)-10-((6-oxo-4-(o-tolyl)pyrimidin-1(6H)-yl)methyl)-7-azaspiro[4.5]decane-7-carboxylate (2.33 g). [α]_{D}²⁵=+77.60 (c 0.1 in CHCl₃); and the second eluted material (R_{T}=23.76 min) afforded title compound (2.26 g). [α]_{D}²⁵=−77.50 (c 0.1 in CHCl₃).

### Intermediate 19: N-((2S,4R)-2-(2,5-Difluorophenyl)piperidin-4-yl)-2,2,2-trifluoro-N-methylacetamide hydrochloride

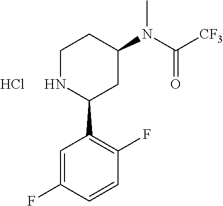 [SMILES: Cl ; CN(C(=O)C(F)(F)F)[C@@H]1CCN[C@H](c2cc(F)ccc2F)C1]

Step 1: Methyl (S)-3-amino-3-(2,5-difluorophenyl)propanoate hydrochloride: To a stirred solution of (S)-3-amino-3-(2,5-difluorophenyl)propanoic acid (10 g, 49.7 mmol) in MeOH (80 mL) was added dropwise SOCl₂ (4.33 mL, 59.7 mmol) at 0° C. and then stirred at rt. After 16 h. the reaction mixture was concentrated under reduced pressure and residue was triturated with pentane and dried under vacuum to give title compound (10 g, 83%). LCMS (Method C): R_{T}=2.72 min, m/z=216 [M+H]⁺.

Step 2: Methyl (S)-3-((1-(2,5-difluorophenyl)-3-(methylperoxy)-3λ²-propyl)amino)propanoate: Methyl (S)-3-amino-3-(2,5-difluorophenyl)propanoate hydrochloride (15.6 g, 62.0 mmol) was dissolved in MeOH (130 mL) and stirred at rt. TEA (12.7 mL, 93.0 mmol) was added followed by dropwise addition of a solution of methyl acrylate (8.43 mL, 93.0 mmol) in MeOH (5 mL). After 16 h, the reaction mixture was concentrated under reduced pressure and diluted with water (500 mL) and extracted with EtOAc (2×750 mL). The organic layer was dried (Na₂SO₄) and concentrated under reduced pressure to give the crude product that was purified by flash chromatography (0-10% EtOAc in hexane) to yield the title compound (12 g, 64%). LCMS (Method C): R_{T}=1.72 min, m/z=302 [M+H]⁺.

Step 3: Methyl (S)-3-((tert-butoxycarbonyl)(3-methoxy-3-oxopropyl)amino)-3-(2,5-difluorophenyl)propanoate: Methyl (S)-3-((1-(2,5-difluorophenyl)-3-(methylperoxy)-3λ²-propyl)amino)propanoate (10.6 g, 35.2 mmol) was suspended in MeOH (90 mL) and Boc₂O (14.6 mL, 63.3 mmol) was added dropwise with stirring at rt. After 16 h, the reaction mixture was diluted with water (750 mL) and extracted with ethyl acetate (2×1 L). The combined organic phase was dried (Na₂SO₄) and concentrated under reduced pressure. The remaining residue was purified by flash chromatography (0-20% EtOAc in hexane) to yield the title compound (12 g, 84%). LCMS (Method C): R_{T}=3.67 min, m/z=402 [M+H]⁺.

Step 4: 1-tert-Butyl 3-methyl (6S)-4-hydroxy-6-(2,5-difluorophenyl)-1,2,5,6-tetrahydropyridine-1,3-dicarboxylate and 1-(tert-butyl) 3-methyl (2S)-2-(2,5-difluorophenyl)-4-hydroxy-3,6-dihydropyridine-1,3(2H)-dicarboxylate: Methyl (S)-3-((tert-butoxycarbonyl)(3-methoxy-3-oxopropyl)amino)-3-(2,5-difluorophenyl)propanoate (15.0 g, 37.4 mmol) was dissolved in toluene (300 mL) and cooled to −78° C. A 1 M solution of LiHMDS in THF (9.38 mL, 56.0 mmol) was added dropwise. After complete addition, the reaction mixture was stirred at −78° C. for 4 h before the reaction was quenched with water (500 mL) and extracted using ethyl acetate (2×750 mL). The organic layer was dried (Na₂SO₄) and concentrated under reduced pressure to give the crude product that was purified by flash chromatography (0-20% EtOAc in hexane) to yield the mixture of isomeric title compounds (8.0 g, 57%). LCMS (Method D): R_{T}=3.49, 3.77 min (2 peaks), m/z=370 [M+H]⁺.

Step 5: tert-Butyl (S)-2-(2,5-difluorophenyl)-4-oxopiperidine-1-carboxylate: A mixture of 1-tert-Butyl 3-methyl (6S)-4-hydroxy-6-(2,5-difluorophenyl)-1,2,5,6-tetrahydropyridine-1,3-dicarboxylate and 1-(tert-butyl) 3-methyl (2S)-2-(2,5-difluorophenyl)-4-hydroxy-3,6-dihydropyridine-1,3(2H)-dicarboxylate (12.5 g, 33.8 mmol) was dissolved in DMSO (62 mL). NaCl (5.93 g, 102 mmol) and water (4 mL) were added to the reaction mixture and heated at 145° C. After 4 h, the reaction mixture was diluted with water (100 mL) and extracted with ethyl acetate (3×500 mL). The combined organic phase was dried (Na₂SO₄) and concentrated under reduced pressure to give the crude product that was purified by flash chromatography (0-20% EtOAc in hexane) to yield title compound (8 g, 75%). LCMS (Method C): R_{T}=3.49 min, m/z=312 [M+H]⁺.

Step 6: tert-Butyl (2S)-2-(2,5-difluorophenyl)-4-(methylamino)piperidine-1-carboxylate: To a stirred solution of tert-butyl (S)-2-(2,5-difluorophenyl)-4-oxopiperidine-1-carboxylate (2.1 g, 6.43 mmol) in MeOH (20 mL) were added 33% (w/w) MeNH₂ in EtOH (7.94 mL, 64.34 mmol) and catalytic AcOH (1-2 drops) at rt. After 2 h, NaBH₃CN (1.21 g, 19.3 mmol) was added. After a further 16 h, the reaction mixture was diluted with water (100 mL) and extracted with ethyl acetate (2×250 mL). The combined organic phase was dried (Na₂SO₄) and concentrated under reduced pressure to give the crude product that was purified by flash chromatography (0-10% MeOH in DCM) to yield the title compound (1.8 g, 81%). LCMS (Method C): R_{T}=2.9 min. m/z=327 [M+H]⁺.

Step 7: tert-Butyl (2S)-2-(2,5-difluorophenyl)-4-(2,2,2-trifluoro-N-methylacetamido)piperidine-1-carboxylate: To a stirred solution of tert-butyl (2S)-2-(2,5-difluorophenyl)-4-(methylamino)piperidine-1-carboxylate (2.5 g, 7.66 mmol) in DCM (25 mL) was added pyridine (6.17 mL, 76.6 mmol) followed by trifluoroacetic anhydride (1.59 mL, 11.5 mmol) at 0° C. The reaction mixture was stirred at rt for 16 h, before it was diluted with water (100 mL) and extracted using ethyl acetate (2×100 mL). The combined organic phase was dried (Na₂SO₄) and concentrated under reduced pressure to give the crude product that was purified by flash chromatography (0-30% EtOAc in hexane) to yield the title compound (2.8 g, 86%).

Step 8: tert-Butyl (2S,4R)-2-(2,5-difluorophenyl)-4-(2,2,2-trifluoro-N-methylacetamido)piperidine-1-carboxylate: tert-Butyl (2S)-2-(2,5-difluorophenyl)-4-(2,2,2-trifluoro-N-methylacetamido)piperidine-1-carboxylate (2.8 g) was separated into the single stereoisomers by chiral HPLC using a Chiralpak IC (20 mm×250 mm, 5 μm) column with isocratic solvent conditions: 95:2.5/2.5 hexane/DCM/EtOH (Flow rate: 18 mL/min) to give the title compound (first eluting isomer: 1.3 g). LCMS (Method C): R_{T}=3.68 min, m/z=423 [M+H]⁺. ¹H NMR at 100° C. (400 MHz, DMSO-d₆): δ 7.17-7.08 (m, 3H), 4.97-4.93 (m, 1H), 4.39 (br s, 1H), 3.92-3.88 (m, 1H), 3.66-3.59 (m, 1H), 2.96 (d, 3H), 2.24-2.12 (m, 1H), 2.06-2.02 (m, 1H), 1.98-1.95 (m, 1H), 1.83 (br s, 1H), 1.24 (s, 9H); and tert-butyl (2S,4S)-2-(2,5-difluorophenyl)-4-(2,2,2-trifluoro-N-methylacetamido)piperidine-1-carboxylate (second eluting isomer: 0.65 g). LCMS (Method C): R_{T}=3.77 min, m/z=423 [M+H]⁺. ¹H NMR at 100° C. (400 MHz, DMSO-d₆): δ 7.26-7.15 (m, 2H), 7.04 (br s, 1H), 5.62-5.61 (d, 1H), 4.32 (br s, 1H), 4.21-4.17 (d, 1H), 3.91-3.88 (br s, 1H), 3.18-3.07 (br s, 1H), 2.67 (s, 3H), 2.32-2.24 (m, 1H), 2.13-2.10 (m, 1H), 1.98-1.17 (m, 2H), 1.38 (s, 9H). [Note: the stereoisomers were confirmed by comparison of ¹H NMR data with close analogues].

Step 9: N-((2S,4R)-2-(2,5-Difluorophenyl)piperidin-4-yl)-2,2,2-trifluoro-N-methylacetamide hydrochloride: tert-Butyl (2S,4R)-2-(2,5-difluorophenyl)-4-(2,2,2-trifluoro-N-methylacetamido)piperidine-1-carboxylate (1.6 g, 3.78 mmol) was suspended in DCM (5 mL) and 4 M HCl in 1,4-dioxane solution (20 mL) was added. The reaction mixture was stirred at rt for 3 h. The solvent was evaporated under reduced pressure and the residue was triturated with DCM and pentane followed by lyophilization to yield the title compound (1.2 g, 99%). LCMS (Method C): R_{T}=2.52 min, m/z=323 [M+H]⁺. ¹H NMR at 100° C. (400 MHz, DMSO-d₆): δ 9.76 (br s, 2H), 7.93 (s, 1H), 7.32-7.29 (m, 2H), 4.75-4.72 (m, 2H), 3.50-3.32 (m, 2H), 3.02 (s, 3H, overlapping water signal), 2.49 (m, 2H, overlapping DMSO signal), 2.00-1.86 (m, 2H).

### Intermediate 20: tert-Butyl cyclopropyl((2S,4R)-2-phenylpiperidin-4-yl)carbamate

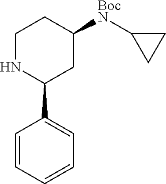 [SMILES: CC(C)(C)OC(=O)N(C1CC1)[C@@H]1CCN[C@H](c2ccccc2)C1]

Step 1: 1-((2S)-4-(Cyclopropylamino)-2-phenylpiperidin-1-yl)-2,2,2-trifluoroethan-1-one: To a stirred solution of (S)-2-phenyl-1-(2,2,2-trifluoroacetyl)piperidin-4-one (2.0 g, 7.26 mmol) in MeOH (10 mL) were added cyclopropylamine (1.5 mL, 21.8 mmol) and catalytic AcOH (1-2 drops) at rt. After 1 h, NaBH₃CN (1.4 g, 21.8 mmol) was added. After a further 16 h, the reaction mixture was diluted with water (100 mL) and extracted using ethyl acetate (2×150 mL). The combined organic phase was dried (Na₂SO₄) and concentrated under reduced pressure to give the crude product that was purified by flash chromatography (0-30% EtOAc in hexane) to yield title compound (1.6 g, 70%). LCMS (Method C): R_{T}=3.33 min, m/z=313 [M+H]⁺.

Step 2: tert-Butyl cyclopropyl((2S)-2-phenyl-1-(2,2,2-trifluoroacetyl)piperidin-4-yl)carbamate: To a stirred solution of 1-((2S)-4-(cyclopropylamino)-2-phenylpiperidin-1-yl)-2,2,2-trifluoroethan-1-one (1.6 g, 5.13 mmol) in DCM (10 mL) were added TEA (2.15 mL, 15.4 mmol) and Boc₂O (1.4 mL, 6.15 mmol) at rt. After 16 h, the reaction mixture was quenched with saturated NaHCO_{3(aq)} solution (10 mL), diluted with water (100 mL) and extracted using ethyl acetate (2×100 mL). The combined organic phase was dried (Na₂SO₄) and concentrated under reduced pressure to give the crude product that was purified by flash chromatography (30% EtOAc in hexane) to yield the title compound (1.5 g, 70%). LCMS (Method D): R_{T}=3.94 min, m/z=413 [M+H]⁺.

Step 3: tert-Butyl cyclopropyl((2S)-2-phenylpiperidin-4-yl)carbamate: To a stirred solution of tert-butyl cyclopropyl((2S)-2-phenyl-1-(2,2,2-trifluoroacetyl)piperidin-4-yl)carbamate (1.5 g, 4.80 mmol) in 4:1 MeOH/water (20 mL) was added K₂CO₃ (0.997 g, 7.21 mmol) at rt. After 16 h, the reaction mixture was diluted with water (50 mL) and extracted using ethyl acetate (2×100 mL). The combined organic phase was dried (Na₂SO₄) and concentrated under reduced pressure to give the crude product that was purified by flash chromatography (0-30% EtOAc in hexane) to yield the title compound (900 mg, 59%).

Step 4: tert-Butyl cyclopropyl((2S,4R)-2-phenylpiperidin-4-yl)carbamate: tert-Butyl cyclopropyl((2S)-2-phenylpiperidin-4-yl)carbamate (900 mg) was separated into the single stereoisomers by chiral HPLC using a Chiralpak IC (20 mm×250 mm, 5 μm) column with isocratic solvent conditions: 95:5 hexane/EtOH (Flow rate: 21 mL/min) to give the title compound (first eluting isomer: 400 mg). LCMS (Method D): R_{T}=3.06 min, m/z=317 [M+H]⁺. ¹H NMR (400 MHz, CDCl₃): δ 7.37-7.24 (m, 5H), 3.92-3.89 (m, 1H), 3.68-3.65 (m, 1H), 3.27-3.24 (m, 1H), 2.83 (t, 1H), 2.31 (br s, 1H), 2.02-1.91 (m, 3H), 1.79-1.76 (m, 1H), 2.45 (s, 9H), 0.74-0.67 (m, 4H); and tert-butyl cyclopropyl((2S,4S)-2-phenylpiperidin-4-yl)carbamate (second eluting isomer: 300 mg). LCMS (Method D): R_{T}=3.19 min, m/z=317 [M+H]⁺. ¹H NMR (400 MHz, CDCl₃): δ 7.49 (d, 2H), 7.34 (t, 2H), 7.21 (t, 1H), 4.35 (s, 1H), 3.87-3.85 (m, 1H), 2.91 (d, 1H), 2.77-2.71 (m, 1H), 2.36-2.32 (m, 3H), 2.12-2.02 (m, 1H), 1.44 (s, 9H), 0.77-0.68 (m, 4H).

### Intermediate 21: N-((2S,4R)-2-(2,4-Difluorophenyl)piperidin-4-yl)-2,2,2-trifluoroacetamide hydrochloride

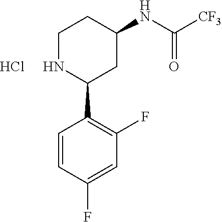 [SMILES: Cl ; O=C(N[C@@H]1CCN[C@H](c2ccc(F)cc2F)C1)C(F)(F)F]

Step 1: tert-Butyl (S)-2-(2,4-difluorophenyl)-4-oxopiperidine-1-carboxylate: The title compound was prepared similarly to tert-butyl (S)-4-oxo-2-phenylpiperidine-1-carboxylate (Intermediate 1, Steps 1 to 5) except using (S)-3-amino-3-(2,4-difluorophenyl)propanoic acid instead of (S)-3-amino-3-phenylpropanoic acid as the starting material. LCMS (Method C): R_{T}=3.45 min, m/z=312 [M+H]⁺. ¹H NMR (400 MHz, DMSO-d₆): δ 7.37-7.31 (m, 1H), 7.26-7.20 (m, 1H), 7.08 (t, 1H), 5.42 (br s, 1H), 4.07-3.99 (m, 1H), 3.69-3.62 (m, 1H), 2.92-2.86 (m, 1H), 2.75-2.70 (m, 1H), 2.48-2.40 (m, 2H), 1.26 (s, 9H).

Step 2: N-((2S,4R)-2-(2,4-Difluorophenyl)piperidin-4-yl)-2,2,2-trifluoroacetamide hydrochloride: The title compound was prepared similarly to 2,2,2-trifluoro-N-((2S,4R)-2-(3-fluorophenyl)piperidin-4-yl)acetamide hydrochloride (Intermediate 11, Steps 1 to 4) except using tert-butyl (S)-2-(2,4-difluorophenyl)-4-oxopiperidine-1-carboxylate instead of tert-butyl (S)-2-(3-fluorophenyl)-4-oxopiperidine-1-carboxylate and the separation of diastereoisomers was possible using flash chromatography. LCMS (Method E): R_{T}=1.81 min. m/z=309 [M+H]. ¹H NMR at 100° C. (400 MHz, DMSO-d₆): δ 9.48 (br s, 1H), 9.36 (br s, 1H), 7.93-7.87 (m, 1H), 7.27-7.17 (m, 2H), 4.66-4.62 (m, 1H), 4.20-4.17 (m, 1H), 3.43 (d, 1H), 3.30-3.14 (m, 1H), 2.19-2.05 (m, 4H).

### Intermediate 22: N-((2S,4R)-2-(2,5-Difluorophenyl)piperidin-4-yl)-2,2,2-trifluoroacetamide hydrochloride

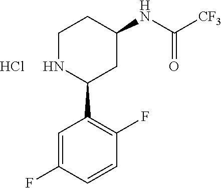 [SMILES: Cl ; O=C(N[C@@H]1CCN[C@H](c2cc(F)ccc2F)C1)C(F)(F)F]

The title compound was prepared similarly to 2,2,2-trifluoro-N-((2S,4R)-2-(3-fluorophenyl)piperidin-4-yl)acetamide hydrochloride (Intermediate 11, Steps 1 to 4) except using tert-butyl (S)-2-(2,5-difluorophenyl)-4-oxopiperidine-1-carboxylate instead of tert-butyl (S)-2-(3-fluorophenyl)-4-oxopiperidine-1-carboxylate and the following conditions for the separation of diastereoisomers: CHIRALCEL OJ-H (20 mm×250 mm, 5 μm) column with isocratic solvent conditions: 95:5 hexane/EtOH (Flow rate: 21 mL/min). LCMS (Method C): R_{T}=2.97 min, m/z=309 [M+H]⁺. ¹H NMR at 100° C. (400 MHz, DMSO-d₆): δ 10.03 (br s, 1H), 9.70 (d, 1H), 7.80 (s, 1H), 7.41-7.34 (m, 2H), 4.71 (br s, 1H), 4.20 (br s, 1H), 3.43 (d, 1H), 2.07-1.96 (m, 4H).

### Intermediate 23: tert-Butyl (2S,4S)-4-azido-5-fluoro-2-phenylpiperidine-1-carboxylate

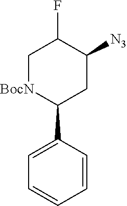 [SMILES: CC(C)(C)OC(=O)N1CC(F)[C@@H](N=[N+]=[N-])C[C@H]1c1ccccc1]

To a stirred solution of tert-butyl (2S,4S,5S)-4-azido-5-hydroxy-2-phenylpiperidine-1-carboxylate (1.02 g, 3.3 mmol) [Prepared according to Angew. Chem. Int. Ed., 2011, 50, 2734-2737 except using commercially available (S)-1-phenylbut-3-en-1-amine to generate tert-butyl (S)-2-phenyl-3,6-dihydropyridine-1(2H)-carboxylate as the desired single stereoisomer to be taken through the subsequent steps described therein] in DCM (30 mL) at −10° C. was added sodium bicarbonate (1.62 g, 19.2 mmol) followed by dropwise addition of 4-morpholinylsulfur trifluoride (0.72 g, 5.2 mmol). After 24 h, the reaction mixture was washed with water (×3), the organic phase was dried (Na₂SO₄), the solvents were removed in vacuo and the remaining residue was purified by preparative HPLC to give the title compound (0.361 g, 35%). LCMS (Method F): R_{T}=2.86 min. m/z=265 [M−butene+H]⁺. ¹H NMR (400 MHz, DMSO-d₆): δ 7.38-7.19 (m, 5H), 5.31-5.24 (m, 1H), 4.85-4.29 (m, 2H), 4.10-3.87 (m, 1H), 3.49-3.32 (m, 1H), 2.66-2.38 (m, 1H), 2.24-2.05 (m, 1H), 1.43-1.38 (d, 9H).

### Intermediate 24: tert-Butyl methyl((3S,5S)-5-phenylpyrrolidin-3-yl)carbamate

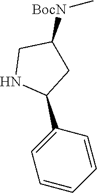 [SMILES: CN(C(=O)OC(C)(C)C)[C@@H]1CN[C@H](c2ccccc2)C1]

Step 1: rac-tert-Butyl ((3S,5S)-1-(4-methoxybenzyl)-5-phenypyrrolidin-3-yl)carbamate: To a stirred solution of rac-tert-butyl ((3S,5S)-5-phenylpyrrolidin-3-yl)carbamate (2.0 g, 7.63 mmol, 1 equiv.) [commercially available] in DCE (60 mL) was added p-methoxybenzaldehyde (1.14 g, 8.40 mmol), acetic acid (0.458 g, 7.63 mmol) and sodium triacetoxyborohydride (4.85 g, 22.9 mmol) at rt. After 48 h, the reaction mixture was diluted by DCM (200 mL), washed by saturated K₂CO_{3(aq)} solution, water, and brine. The volatiles were removed in vacuo and the remaining residue was purified by flash chromatography (0-100%, EtOAc in hexane) to give the title compound (2.65 g, 91%). LCMS (Method F): R_{T}=1.01 min, m/z=383 [M+H]⁺. ¹H NMR (400 MHz, DMSO-d₆): δ 1.28-1.37 (m, 1OH), 1.52-1.66 (m, 1H), 2.39 (t, J=9.9 Hz, 2H), 2.72 (d, J=9.9 Hz, 1H), 2.92 (d, J=13.2 Hz, 1H), 3.54 (d, J=13.2 Hz, 1H), 3.71 (s, 3H), 3.89-3.99 (m, 1H), 6.84 (d, J=8.0 Hz, 2H), 7.06 (d, J=6.8 Hz, 1H), 7.14 (d, J=8.0 Hz, 2H), 7.25 (t, J=6.8 Hz, 1H), 7.35 (t, J=7.4 Hz, 2H), 7.49 (d, J=7.4 Hz, 2H).

Step 2: tert-Butyl ((3S,5S)-1-(4-methoxybenzyl)-5-phenylpyrrolidin-3-yl)carbamate: rac-tert-Butyl ((3S,5S)-1-(4-methoxybenzyl)-5-phenylpyrrolidin-3-yl)carbamate (2.5 g) was resolved into the single stereoisomers by chiral SFC using a Chiralcel OJ-H (20 mm×250 mm, 5 μm) column with isocratic solvent conditions: 90:10 CO₂/MeOH (Flow rate: 50 mL/min). The first eluted material (R_{T}=11.63 min) afforded the title compound (1.20 g). A sample of this material (40 mg) was subjected to N-PMB deprotection under the conditions of General Procedure 9 and optical rotation of the product was measured to confirm that it matched that of tert-butyl ((3S,5S)-5-phenylpyrrolidin-3-yl)carbamate (Intermediate 15). Hence, the first eluted material was confirmed as the title compound and carried through to the next step. The second eluted material (R_{T}=13.79 min) afforded tert-butyl ((3R,5R)-1-(4-methoxybenzyl)-5-phenylpyrrolidin-3-yl)carbamate (501 mg).

Step 3: (3S,5S)-1-(4-Methoxybenzyl)-N-methyl-5-phenylpyrrolidin-3-amine: To a stirred solution of tert-butyl ((3S,5S)-1-(4-methoxybenzyl)-5-phenylpyrrolidin-3-yl)carbamate (0.76 g, 1.99 mmol) in THF (20 mL) was added LiAlH₄ (0.2 g, 5.26 mmol) portionwise and resultant mixture was refluxed. After 5 h, the reaction mixture was cooled to 0° C. and quenched using 35% NaOH_{(aq)} solution. The resulting suspension was filtered, washed using hot THF, the solvents were removed in vacuo to give the title compound (0.47 g, 79%) as yellow oil. LCMS (Method F): R_{T}=0.86 min, m/z=297 [M+H]⁺.

Step 4: tert-Butyl ((3S,5S)-1-(4-methoxybenzyl)-5-phenylpyrrolidin-3-yl)(methyl)carbamate: To a stirred solution of (3S,5S)-1-(4-methoxybenzyl)-N-methyl-5-phenylpyrrolidin-3-amine (0.51 g, 1.72 mmol) in methanol (15 mL) was added Boc₂O (0.417 g, 1.91 mmol) at rt. After 16 h, the volatiles were removed in vacuo to give the crude title compound (0.68 g, quantative) as yellow oil that was taken into the next step without purification. LCMS (Method F): R_{T}=1.11 min, m/z=397 [M+H]⁺.

Step 5: tert-Butyl methyl((3S,5S)-5-phenylpyrrolidin-3-yl)carbamate: To a stirred solution of tert-butyl ((3S,5S)-1-(4-methoxybenzyl)-5-phenylpyrrolidin-3-yl)(methyl)carbamate (0.68 g, 1.72 mmol) was dissolved in acetonitrile (13.5 mL) and a solution of ceric ammonium nitrate (3.76 g, 6.67 mmol) in water (13.5 mL) was added dropwise at 0° C. After 18 h, the volatiles were removed in vacuo, the remaining mixture was basified using excess K₂CO₃ and extracted using MTBE. The solvents were removed in vacuo and the crude material (0.37 g, 52% pure) was purified by preparative HPLC to afford the title compound (11.5 mg, 2.4%). LCMS (Method F): R_{T}=0.98 min, m/z=277 [M+H]⁺.

### Intermediate 25: tert-Butyl (R)-10-((4-(2-methoxyphenyl)-6-oxopyrimidin-1(6H)-yl)methyl)-7-azaspiro[4.5]decane-7-carboxylate

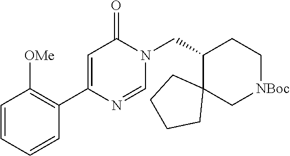 [SMILES: COc1ccccc1-c1cc(=O)n(C[C@@H]2CCN(C(=O)OC(C)(C)C)CC23CCCC3)cn1]

The title compound was prepared similarly to tert-butyl (R)-10-((6-oxo-4-(o-tolyl)pyrimidin-1(6H)-yl)methyl)-7-azaspiro[4.5]decane-7-carboxylate (Intermediate 18) except using 6-(2-methoxyphenyl)pyrimidin-4(3H)-one [commercially available] instead of 6-(o-tolyl)pyrimidin-4(3H)-one in the first step. tert-Butyl 10-((4-(2-methoxyphenyl)-6-oxopyrimidin-1(6H)-yl)methyl)-7-azaspiro[4.5]decane-7-carboxylate (3.75 g) was resolved into the single stereoisomers by chiral HPLC using a Chiralpak IG (20 mm×250 mm, 5 μm) column with isocratic solvent conditions: 60:20:20 hexane/IPA/MeOH (Flow rate: 12 mL/min). The first eluted material (R_{T}=26.2 min) afforded tert-butyl (S)-10-((4-(2-methoxyphenyl)-6-oxopyrimidin-1(6H)-yl)methyl)-7-azaspiro[4.5]decane-7-carboxylate (1.77 g). [α]_{D}²⁵=+7.28 (c 0.1 in CHCl₃); and the second eluted material (R_{T}=32.0 min) afforded the title compound (1.82 g). LCMS (Method F): R_{T}=1.63 min, m/z=454 [M+H]⁺. [α]_{D}²⁵=−9.76 (c 0.1 in CHCl₃).

### Intermediate 26: N-((2S,4R)-2-(2,4-Difluorophenyl)piperidin-4-yl)-2,2,2-trifluoro-N-methylacetamide hydrochloride

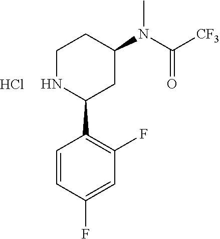 [SMILES: Cl ; CN(C(=O)C(F)(F)F)[C@@H]1CCN[C@H](c2ccc(F)cc2F)C1]

The title compound was prepared similarly to N-((2S,4R)-2-(2,5-difluorophenyl)piperidin-4-yl)-2,2,2-trifluoro-N-methylacetamide hydrochloride (Intermediate 19) except using (S)-3-amino-3-(2,4-difluorophenyl)propanoic acid instead of (S)-3-amino-3-(2,5-difluorophenyl)propanoic acid as the starting material. tert-Butyl (2S)-2-(2,4-difluorophenyl)-4-(2,2,2-trifluoro-N-methylacetamido)piperidine-1-carboxylate (2.0 g) was separated by reversed phase preparative HPLC (C18 column) to give tert-butyl (2S,4R)-2-(2,4-difluorophenyl)-4-(2,2,2-trifluoro-N-methylacetamido)piperidine-1-carboxylate (1^{st} eluting isomer: 1.0 g, de=100%). [Note: cis and trans stereochemistry was assigned by nOe data]. LCMS (Method C): R_{T}=3.73 min, m/z=423 [M+H]⁺. This material was deprotected to give the title compound (610 mg). LCMS (Method E): R_{T}=1.81 min, m/z=323 [M+H]⁺. ¹H NMR at 100° C. (400 MHz, DMSO-d₆): δ 9.72 (br s, 2H), 8.11-8.05 (m, 1H), 7.26-7.16 (m, 2H), 4.72-4.69 (m, 2H), 3.48-3.40 (m, 2H), 3.02 (s, 3H, overlapping water signal), 2.49 (m, 1H, overlapping DMSO signal), 2.00-1.85 (m, 2H).

### Intermediate 27: tert-Butyl (R)-10-((6-fluoro-4-oxoquinazolin-3(4H)-yl)methyl)-7-azaspiro[4.5]decane-7-carboxylate

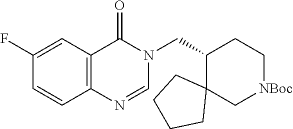 [SMILES: CC(C)(C)OC(=O)N1CC[C@@H](Cn2cnc3ccc(F)cc3c2=O)C2(CCCC2)C1]

The title compound was prepared similarly to tert-butyl (R)-10-((6-oxo-4-(o-tolyl)pyrimidin-1(6H)-yl)methyl)-7-azaspiro[4.5]decane-7-carboxylate (Intermediate 18) except using 6-fluoroquinazolin-4(3H)-one [commercially available] instead of 6-(o-tolyl)pyrimidin-4(3H)-one in the first step. tert-Butyl 10-((6-fluoro-4-oxoquinazolin-3(4H)-yl)methyl)-7-azaspiro[4.5]decane-7-carboxylate (2.46 g) was resolved into the single stereoisomers by chiral HPLC using a CHIRAL ART Cellulose-SC (20 mm×250 mm, 5 μm) column with isocratic solvent conditions: 80:10:10 hexane/IPA/MeOH (Flow rate: 13 mL/min). The first eluted material (R_{T}=12.9 min) afforded tert-butyl (S)-10-((6-fluoro-4-oxoquinazolin-3(4H-yl)methyl)-7-azaspiro[4.5]decane-7-carboxylate (1.11 g). [α]_{D}²⁵=+77.96 (c 0.25 in CHCl₃); and the second eluted material (R_{T}=16.9 min) afforded the title compound (1.09 g). LCMS (Method F): R_{T}=4.13 min, m/z=360 [M−butene+H]⁺. ¹H NMR (DMSO-d₆) δ: 8.37 (s, 1H), 7.83-7.71 (m, 3H), 4.18 (m, 1H), 3.80 (m, 1H), 3.67 (m, 1H), 3.52 (m, 1H), 2.82-2.55 (m, 2H), 1.79 (m, 2H), 1.65 (m, 2H), 1.53 (m, 2H), 1.38 (s, 9H), 1.40-1.14 (m, 5H). [α]_{D}²⁵=−77.36 (c 0.25 in CHCl₃).

### Intermediate 28: tert-Butyl ((2S,4R)-2-(2-fluorophenyl)piperidin-4-yl)(methyl)carbamate

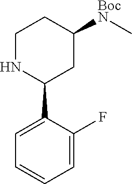 [SMILES: CN(C(=O)OC(C)(C)C)[C@@H]1CCN[C@H](c2ccccc2F)C1]

Step 1: rac-(2S,4R)-1-((Benzyloxy)carbonyl)-2-(2-fluorophenyl)piperidine-4-carboxylic acid: To a stirred solution of rac-methyl (2S,4R)-2-(2-fluorophenyl)piperidine-4-carboxylate [Prepared according to the procedure for Intermediate 17, Steps 1-3 except using (2-fluorophenyl)boronic acid (21 g, 150 mmol) instead of phenylboronic acid to react with methyl 2-bromoisonicotinate (21.6 g, 100 mmol)] in 2:1 1,4-dioxane/water was added sodium hydroxide (4 equiv.) at 0° C., followed by dropwise addition of CbzCl (1.5 equiv.) in 1,4-dioxane. After 16 h, the solvents were removed in vacuo, and the remaining residue was diluted with water and the aqueous mixture was washed using MTBE (×2), then acidified using saturated NaHSO_{4(aq)} solution to ˜pH 2 and extracted using EtOAc. The solvents were removed in vacuo to give the title compound (12.1 g, 33.9 mmol) that was used in the next step without further purification.

Step 2: rac-Benzyl (2S,4R)-4-((tert-butoxycarbonyl)(methyl)amino)-2-(2-fluorophenyl)piperidine-1-carboxylate: rac-(2S,4R)-1-((benzyloxy)carbonyl)-2-(2-fluorophenyl)piperidine-4-carboxylic acid (12.1 g, 33.9 mmol) was dissolved in toluene (242 mL) and treated with diphenylphosphorylazide (11.2 g, 40.7 mmol) and triethylamine (4.11 g, 40.7 mmol). The reaction mixture was heated at 75° C. for 4 h, followed by addition of trimethylsilylethanol (12.0 g, 102 mmol). The reaction mixture was heated at 100° C. After 24 h, the solvents were removed in vacuo and the crude material was purified by flash chromatography to give rac-benzyl (2S,4R)-2-(2-fluorophenyl)-4-(((2-(trimethylsilyl)ethoxy)carbonyl)amino)piperidine-1-carboxylate (9.80 g, 61%) that was dissolved in THF (98 mL) and 1 M TBAF in THF solution (35.3 mL, 35.3 mmol, 1.7 equiv.) was added and the reaction mixture was heated at 50° C. After 18 h, the solvents were removed in vacuo and the remaining residue was dissolved in EtOAc (320 mL), washed sequentially with water, brine, and dried (Na₂SO₄). The solvents were removed in vacuo to give crude rac-benzyl (2S,4R)-4-amino-2-(2-fluorophenyl)piperidine-1-carboxylate (6.13 g, 90%) that was subsequently treated Boc₂O (5.0 g, 23.4 mmol, 1.25 equiv.) in methanol (125 mL) and stirred at rt. After 16 h, the solvents were removed in vacuo and the remaining residue was purified by flash chromatography to give rac-benzyl (2S,4R)-4-((tert-butoxycarbonyl)amino)-2-(2-fluorophenyl)piperidine-1-carboxylate (2.2 g, 28%) that was subsequently dissolved in DMF (22 mL) and cooled to 0° C. Sodium hydride (60% in mineral oil, 0.26 g, 5.92 mmol, 1.15 equiv.) and iodomethane (0.95 g, 6.68 mmol, 1.3 equiv.) were added and the reaction mixture was stirred at rt. After 18 h, the reaction mixture was diluted with water (85 mL) and quenched by saturated NH₄Cl_{(aq)} solution. The resultant mixture was extracted using MTBE (×3), the combined organic phase concentrated and the remaining residue was purified by flash chromatography to give the title compound (1.9 g, 84%). LCMS (Method F): R_{T}=1.49 min, m/z=443 [M+H]⁺. ¹H NMR (400 MHz, DMSO-d₆): δ 7.28 (m, 5H), 7.16 (m, 2H), 7.05 (m, 2H), 4.97 (m, 3H), 4.00 (m, 1H), 3.57 (m, 1H), 3.31 (m, 1H), 2.66 (s, 3H), 2.04 (m, 2H), 1.85 (m, 1H), 1.67 (m, 1H), 1.37 (s, 9H).

Step 3: Benzyl (2S,4R)-4-((tert-butoxycarbonyl)(methyl)amino)-2-(2-fluorophenyl)piperidine-1-carboxylate: rac-Benzyl (2S,4R)-4-((tert-butoxycarbonyl)(methyl)amino)-2-(2-fluorophenyl)piperidine-1-carboxylate (1.9 g) was resolved into the single stereoisomers by chiral HPLC using a Chiralpak AD-H (20 mm×250 mm, 5 μm) column with isocratic solvent conditions: 80:10:10 hexane/IPA/MeOH (Flow rate: 12 mL/min). The first eluted material (R_{T}=12.32 min) afforded benzyl (2R,4S)-4-((tert-butoxycarbonyl)(methyl)amino)-2-(2-fluorophenyl)piperidine-1-carboxylate (0.85 g) and the second eluted material (R_{T}=15.35 min) afforded the title compound (0.84 g).

Step 4: tert-Butyl ((2S,4R)-2-(2-fluorophenyl)piperidin-4-yl)(methyl)carbamate: To a stirred solution of benzyl (2S,4R)-4-((tert-butoxycarbonyl)(methyl)amino)-2-(2-fluorophenyl)piperidine-1-carboxylate (0.84 g, 1.90 mmol) in methanol (30 mL) was added 10% (w/w) Pd/C (80 mg, 40 mol %) and the reaction mixture was hydrogenated (˜1 atm, balloon) at rt. After 24 h, the reaction mixture was filtered and evaporated to dryness to give the title compound (0.43 g, 74%). LCMS (Method F): R_{T}=1.00 min, m/z=309 [M+H]⁺. ¹H NMR (400 MHz, CDCl₃): δ 7.50 (m, 1H), 7.20 (m, 1H), 7.10 (m, 1H), 6.98 (m, 1H), 4.05 (m, 2H), 3.25 (m, 1H), 2.85 (m, 1H), 2.72 (s, 3H), 2.08 (m, 1H), 1.85 (dd, 1H), 1.70 (m, 3H), 1.45 (s, 9H). [α]_{D}²⁵=+44.6 (c 0.35 in MeOH).

### Intermediate 29: tert-Butyl ((2S,4R)-2-(4-fluorophenyl)piperidin-4-yl)(methyl)carbamate

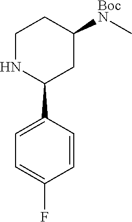 [SMILES: CN(C(=O)OC(C)(C)C)[C@@H]1CCN[C@H](c2ccc(F)cc2)C1]

The title compound was prepared according to the procedure for Intermediate 28 except using (4-fluorophenyl)boronic acid instead of (2-fluorophenyl)boronic acid and following the steps to prepare rac-benzyl (2S,4R)-4-((tert-butoxycarbonyl)(methyl)amino)-2-(4-fluorophenyl)piperidine-1-carboxylate (2.2 g) that was resolved into the single stereoisomers by chiral HPLC using a Chiralpak AD-H (20 mm×250 mm, 5 μm) column with isocratic solvent conditions: 80:10:10 hexane/IPA/MeOH (Flow rate: 12 mL/min). The first eluted material (R_{T}=15.85 min) afforded benzyl (2S,4R)-4-((tert-butoxycarbonyl)(methyl)amino)-2-(4-fluorophenyl)piperidine-1-carboxylate (996 mg) and the second eluted material (R_{T}=33.58 min) afforded benzyl (2R,4S)-4-((tert-butoxycarbonyl)(methyl)amino)-2-(4-fluorophenyl)piperidine-1-carboxylate (1.08 g). Benzyl (2S,4R)-4-((tert-butoxycarbonyl)(methyl)amino)-2-(4-fluorophenyl)piperidine-1-carboxylate (996 mg) was used in the final step (Cbz deprotection) to give the title compound (0.58 g, 84%). LCMS (Method F): R_{T}=0.82 min, m/z=309 [M+H]⁺. ¹H NMR (400 MHz, DMSO-d₆): δ 7.66 (m, 2H), 7.24 (t, 2H), 4.30 (m, 2H), 3.31 (m, 1H), 3.05 (m, 1H), 2.67 (s, 3H), 2.12 (q, 2H), 1.70 (dd, 2H), 1.38 (s, 9H). [α]_{D}²⁵=+46.5 (c 0.25 in MeOH).

### Intermediate 30: N-((2S,4R)-2-(3,5-Difluorophenyl)piperidin-4-yl)-2,2,2-trifluoroacetamide hydrochloride

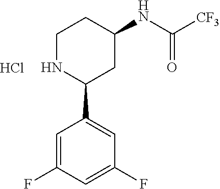 [SMILES: Cl ; O=C(N[C@@H]1CCN[C@H](c2cc(F)cc(F)c2)C1)C(F)(F)F]

tert-Butyl (S)-2-(3,5-difluorophenyl)-4-oxopiperidine-1-carboxylate was prepared similarly to tert-butyl (S)-2-(2,5-difluorophenyl)-4-oxopiperidine-1-carboxylate (Intermediate 19, Steps 1 to 5) except using (S)-3-amino-3-(3,5-difluorophenyl)propanoic acid instead of (S)-3-amino-3-(2,5-difluorophenyl)propanoic acid. The title compound was prepared similarly to 2,2,2-trifluoro-N-((2S,4R)-2-(3-fluorophenyl)piperidin-4-yl)acetamide hydrochloride (Intermediate 11, Steps 1 to 4) except using tert-butyl (S)-2-(3,5-difluorophenyl)-4-oxopiperidine-1-carboxylate instead of tert-butyl (S)-2-(3-fluorophenyl)-4-oxopiperidine-1-carboxylate and the diastereoisomers in the analogous Step 3 were separated using reversed phase preparative HPLC (C18 column) instead of chiral preparative HPLC methods. LCMS (Method C): R_{T}=1.82 min, m/z=309 [M+H]⁺. ¹H NMR (400 MHz, DMSO-d₆): δ 9.71 (br s, 1H), 9.38 (d, 1H), 7.41 (t, 2H), 7.21-7.16 (m, 1H), 4.48-4.45 (m, 1H), 4.16-4.08 (m, 1H), 3.58-3.42 (m, 1H), 3.19-3.12 (m, 1H), 2.19-2.01 (m, 4H).

### Intermediate 31: tert-Butyl (R)-10-((4-(2-fluorophenyl)-6-oxopyrimidin-1(6H)-yl)methyl)-7-azaspiro[4.5]decane-7-carboxylate

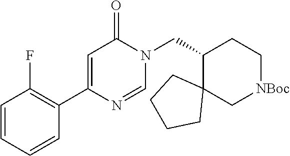 [SMILES: CC(C)(C)OC(=O)N1CC[C@@H](Cn2cnc(-c3ccccc3F)cc2=O)C2(CCCC2)C1]

The title compound was prepared similarly to tert-butyl (R)-10-((6-oxo-4-(o-tolyl)pyrimidin-1(6H)-yl)methyl)-7-azaspiro[4.5]decane-7-carboxylate (Intermediate 18) except using 6-(2-fluorophenyl)pyrimidin-4(3H)-one [commercially available] instead of 6-(o-tolyl)pyrimidin-4(3H)-one in the first step. tert-Butyl 10-((4-(2-fluorophenyl)-6-oxopyrimidin-1(6H)-yl)methyl)-7-azaspiro[4.5]decane-7-carboxylate (0.53 g) was resolved into the single stereoisomers by chiral HPLC using a Chiralpak IB (20 mm×250 mm, 5 μm) column with isocratic solvent conditions: 75:15:15 hexane/IPA/MeOH (Flow rate: 15 mL/min). The first eluted material (R_{T}=6.70 min) afforded tert-butyl (S)-10-((4-(2-fluorophenyl)-6-oxopyrimidin-1(6H)-yl)methyl)-7-azaspiro[4.5]decane-7-carboxylate (0.18 g). [α]_{D}²⁵=+70.15 (c 0.25 in CHCl₃); and the second eluted material (R_{T}=7.98 min) afforded the title compound (0.17 g). LCMS (Method F): R_{T}=3.66 min, m/z=386 [M−butene+H]⁺. [α]_{D}²⁵=−60.15 (c 0.25 in CHCl₃).

### Intermediate 32: 4-((2S,4R)-2-Phenylpiperidin-4-yl)morpholine hydrochloride

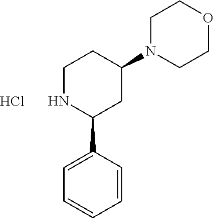 [SMILES: Cl ; c1ccc([C@@H]2C[C@H](N3CCOCC3)CCN2)cc1]

Step 1: tert-Butyl (2S)-4-morpholino-2-phenylpiperidine-1-carboxylate: To a stirred solution of tert-butyl (S)-4-oxo-2-phenylpiperidine-1-carboxylate (800 mg, 2.9 mmol) in MeOH (10 mL) were added morpholine (0.75 mL, 8.72 mmol) and a catalytic amount of AcOH (1-2 drops) at rt. After 1 h, NaBH₃CN (550 mg, 8.72 mmol) was added. After a further 16 h, the reaction mixture was diluted with water (50 mL) and extracted using ethyl acetate (2×100 mL). The combined organic phase was dried (Na₂SO₄) and concentrated under reduced pressure to give the crude product that was purified by flash chromatography (0-10% MeOH in DCM) to yield title compound (800 mg, 80%). LCMS (Method D): R_{T}=3.29 min, m/z=347 [M+H]⁺.

Step 2: tert-Butyl (2S,4R)-4-morpholino-2-phenylpiperidine-1-carboxylate: tert-Butyl (2S)-4-morpholino-2-phenylpiperidine-1-carboxylate (900 mg) was separated into the single stereoisomers by chiral HPLC using a Chiralpak IC (20 mm×250 mm, 5 μm) column with isocratic solvent conditions: 85/15 hexane/EtOH+0.1% isopropylamine (Flow rate: 18 mL/min) to give tert-butyl (2S,4S)-4-morpholino-2-phenylpiperidine-1-carboxylate (first eluting isomer: 90 mg). LCMS (Method D): R_{T}=3.33 min, m/z=347 [M+H]⁺. ¹H NMR (400 MHz, DMSO-d₆): δ 7.37 (t, 2H), 2.23 (t, 1H), 7.17 (t, 2H), 5.41 (br s, 1H), 4.05-4.01 (m, 1H), 3.54 (t, 4H), 2.71 (br s, 1H), 2.45-2.38 (m, 4H), 2.18-2.15 (m, 1H), 1.73-1.65 (m, 2H), 1.39 (s, 9H), 1.32-1.28 (m, 1H); and the title compound (second eluting isomer: 450 mg). LCMS (Method D): R_{T}=3.29 min, m/z=347 [M+H]⁺. ¹H NMR (400 MHz, DMSO-d₆): δ 7.29 (t, 2H), 7.22-7.17 (m, 3H), 4.73-4.69 (m, 1H), 3.91-3.86 (m, 1H), 3.46-3.34 (m, 4H), 2.35-2.32 (m, 4H), 2.05-1.99 (m, 1H), 1.85-1.71 (m, 2H), 1.66-1.63 (m, 1H), 1.21 (s, 9H). [Note: cis and trans stereochemistry was assigned by nOe data].

Step 3: 4-((2S,4R)-2-Phenylpiperidin-4-yl)morpholine hydrochloride: To a stirred solution of tert-butyl (2S,4R)-4-morpholino-2-phenylpiperidine-1-carboxylate (450 mg, 1.30 mmol) in DCM (3 mL) was added 4 M HCl in 1,4-dioxane (10 mL) at rt for 16 h. The solvent was evaporated under reduced pressure and residue was triturated with pentane to yield the title compound (320 mg, 87%). LCMS (Method E): R_{T}=0.14 min, m/z=247 [M+H]⁺. ¹H NMR (400 MHz, DMSO-d₆): δ 11.64 (br s, 1H), 9.75 (d, 1H), 9.51 (d, 1H), 7.62 (d, 2H), 7.50-7.42 (m, 3H), 4.35 (t, 1H), 3.99-3.84 (m, 4H), 3.61 (br s, 1H), 3.52 (br s, 2H), 3.12-3.10 (m, 3H), 2.53 (br s, 1H, merged with DMSO), 2.38-2.16 (m, 3H).

### Intermediate 33: (2S,4R)—N-(3-Methyloxetan-3-yl)-2-phenylpiperidin-4-amine 2,2,2-trifluoroacetate

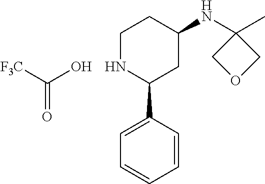

Step 1: tert-Butyl (2S)-4-((3-methyloxetan-3-yl)amino)-2-phenylpiperidine-1-carboxylate: To a stirred solution of tert-butyl-4-oxo-2-phenylpiperidine-1-carboxylate (600 mg, 2.18 mmol) in MeOH (20 mL) was added 3-methyloxetan-3-amine (569 mg, 6.54 mmol) followed by a catalytic amount of AcOH (1-2 drops) at rt. After 3 h, NaBH₃CN (412 mg, 6.54 mmol) was added portionwise at 0° C. and the temperature was allowed to increase to rt. After 16 h, the reaction mixture was diluted with DCM (500 mL), washed with water (100 mL), brine (150 mL), and dried (Na₂SO₄). The solvents were evaporated under reduced pressure to give the crude product that was purified by flash chromatography (0-50% EtOAc in hexane) to yield the title compound (520 mg, 69%). LCMS (Method C): R_{T}=3.17 min, m/z=347 [M+H]⁺.

Step 2: tert-Butyl (2S,4R)-4-((3-methyloxetan-3-yl)amino)-2-phenylpiperidine-1-carboxylate: tert-Butyl (2S)-4-((3-methyloxetan-3-yl)amino)-2-phenylpiperidine-1-carboxylate (600 mg) was separated into the single stereoisomers by chiral HPLC using a Lux i-Amylose-3 (21.2 mm×250 mm, 5 μm) column with isocratic solvent conditions: 80:20 CO₂/MeOH+0.3% isopropylamine (Flow rate: 30 g/min) to give the title compound (second eluting isomer: 140 mg). LCMS (Method D): R_{T}=3.17 min, m/z=347 [M+H]⁺. ¹H NMR (400 MHz, CDCl₃): δ 7.33-7.12 (m, 5H), 4.99 (t, 1H, J=6.8 Hz), 4.25-4.28 (m, 4H), 4.02-3.98 (m, 1H), 3.40-3.33 (m, 1H), 3.08 (bs, 1H), 3.09-1.88 (m, 4H), 1.46 (s, 3H). 1.30 (s, 9H); and tert-butyl (2S,4S)-4-((3-methyloxetan-3-yl)amino)-2-phenylpiperidine-1-carboxylate (first eluting isomer: 140 mg). LCMS (Method D): R_{T}=3.17 min, m/z=347 [M+H]⁺. ¹H NMR (400 MHz, CDCl₃): δ 7.36-7.29 (m, 2H), 7.19-7.17 (m, 3H), 5.54-5.51 (m, 1H), 4.49-4.44 (m, 2H), 4.40-4.34 (m, 2H), 4.17-4.11 (m, 1H), 2.80-2.74 (m, 2H), 2.43-2.39 (m, 1H), 1.62 (m, 2H), 1.45 (s, 12H), 1.32-1.29 (m, 1H). [Note: Stereochemistry was confirmed by nOe data].

Step 3: (2S,4R)—N-(3-Methyloxetan-3-yl)-2-phenylpiperidin-4-amine 2,2,2-trifluoroacetate: To a stirred solution of tert-butyl (2S,4R)-4-((3-methyloxetan-3-yl)amino)-2-phenylpiperidine-1-carboxylate (130 mg, 0.36 mmol) in DCM (10 mL) was added TFA (0.5 mL) dropwise at 0° C. The temperature was allowed to increase to rt and after 5 h, the reaction mixture was concentrated under reduced pressure at low temperature (i.e. ≤35° C.) followed by trituration with pentane/diethyl ether to give title compound (135 mg, 76%). LCMS (Method G): R_{T}=1.38 min, m/z=247 [M+H]⁺. ¹H NMR (400 MHz, DMSO-d₆): δ 9.94 (br s, 1H), 9.32 (br s, 1H), 9.22 (br s, 1H), 7.51-7.42 (m, 5H), 4.77-4.73 (m, 2H), 4.41-4.38 (m, 3H), 3.52-3.45 (m, 2H), 3.22-3.16 (m, 1H), 2.10-2.02 (m, 3H), 1.95-1.89 (m, 1H), 1.71 (s, 3H).

### Intermediate 34: (2S,4R)—N-(3-(Fluoromethyl)oxetan-3-yl)-2-phenylpiperidin-4-amine

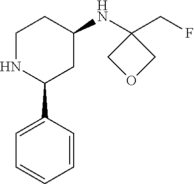 [SMILES: FCC1(N[C@@H]2CCN[C@H](c3ccccc3)C2)COC1]

The title compound was prepared according to the procedure for (2S,4R)—N-(3-methyloxetan-3-yl)-2-phenylpiperidin-4-amine TFA salt (Intermediate 33) except reacting 3-(fluoromethyl)oxetan-3-amine (801 mg, 7.64 mmol) instead of 3-methyloxetan-3-amine with tert-butyl-4-oxo-2-phenylpiperidine-1-carboxylate (700 mg, 2.55 mmol) and the following conditions for the separation of diastereoisomers: Chiralpak IG (21 mm×250 mm, 5 μm) column with isocratic solvent conditions: 70:30 hexane/EtOH (Flow rate: 21 mL/min). LCMS (Method I): R_{T}=0.33 min, m/z=265 [M+H]⁺. ¹H NMR (400 MHz, DMSO-d₆): δ 9.06 (br s, 1H), 8.90 (br s, 1H), 7.51-7.42 (m, 5H), 5.00-4.88 (m, 2H), 4.59 (br s, 2H), 4.49-4.44 (m, 2H), 4.36-4.34 (m, 1H), 3.47-3.40 (m, 2H), 3.15-3.12 (m, 1H), 2.05-1.96 (m, 2H), 1.86-1.73 (m, 2H).

### Intermediate 35: N-((2S,4R)-2-(3,5-Difluorophenyl)piperidin-4-yl)-2,2,2-trifluoro-N-methylacetamide hydrochloride

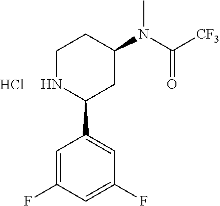 [SMILES: Cl ; CN(C(=O)C(F)(F)F)[C@@H]1CCN[C@H](c2cc(F)cc(F)c2)C1]

The title compound was prepared similarly to N-((2S,4R)-2-(2,5-difluorophenyl)piperidin-4-yl)-2,2,2-trifluoro-N-methylacetamide hydrochloride (Intermediate 19) except using (S)-3-amino-3-(3,5-difluorophenyl)propanoic acid instead of (S)-3-amino-3-(2,5-difluorophenyl)propanoic acid and the diastereoisomers in the analogous Step 8 were separated using reversed phase preparative HPLC (C18 column) instead of chiral preparative HPLC methods. LCMS (Method H): R_{T}=1.86 min, m/z=323 [M+H]⁺. ¹H NMR at 100° C. (400 MHz, DMSO-d₆): δ 9.74 (br s, 1H), 7.48 (t, 2H), 7.21-7.17 (m, 1H), 4.62-4.52 (m, 2H), 3.50-3.46 (m, 1H), 3.25-3.18 (m, 2H), 3.05 (s, 3H), 2.5-2.49 (m, 2H), 2.03 (d, 1H), 1.87 (d, 1H).

### Intermediate 36: tert-Butyl 10-((4-(2-methoxyphenyl)-6-oxo-3,6-dihydropyridin-1(2H)-yl)methyl)-7-azaspiro[4.5]decane-7-carboxylate

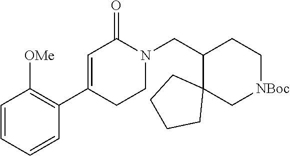 [SMILES: COc1ccccc1C1=CC(=O)N(CC2CCN(C(=O)OC(C)(C)C)CC23CCCC3)CC1]

Step 1: tert-Butyl 10-((6-oxo-4-(((trifluoromethyl)sulfonyl)oxy)-3,6-dihydropyridin-1(2H)-yl)methyl)-7-azaspiro[4.5]decane-7-carboxylate: To a stirred solution of tert-butyl 10-(aminomethyl)-7-azaspiro[4.5]decane-7-carboxylate (5.00 g, 18.6 mmol) [commercially available] in methanol (150 mL) was added methylacrylate (1.60 g, 18.6 mmol) dropwise at 0° C. After 24 h, the volatiles were removed in vacuo and the remaining residue was dissolved in DCM (150 mL), triethylamine (2.07 g, 20.5 mmol) was added, followed by dropwise addition of methyl 3-chloro-3-oxopropanoate (2.80 g, 20.5 mmol) at 0° C. After 24 h, the reaction mixture was washed with water (×3) and dried (Na₂SO₄). The solvents were removed in vacuo and the crude product was dissolved in a solution of sodium methoxide (4.02 g, 74.4 mmol) in methanol (100 mL) and heated to reflux. After 48 h, the reaction mixture was concentrated and carefully acidified using acetic acid. The crude product was extracted using DCM (×3), the solvents were removed in vacuo and the remaining residue was dissolved in 1:1 acetonitrile/water and heated to reflux. After a further 48 h, the reaction mixture was evaporated to dryness and the crude product dissolved in dry THF (150 mL) followed by addition of potassium tert-butoxide (2.03 g, 18 mmol) and N-phenyl-bis(trifluoromethanesulfonimide) (6.47 g, 18 mmol). After 24 h, the solvents were evaporated and the remaining residue was dissolved in DCM and washed with saturated NH₄Cl_{(aq)} solution. The organic phase was dried (Na₂SO₄), the solvents were removed in vacuo and the crude product was purified by flash chromatography to give the title compound (3.40 g, 45% over 5 steps). LCMS (Method F): R_{T}=1.40 min, m/z=396 [M−Boc+H]⁺.

¹H NMR (400 MHz, CDCl₃): δ 7.97 (s, 1H), 6.26 (s, 1H), 4.25 (m, 1H), 4.00 (m, 1H), 3.66 (m, 1H), 3.48 (m, 1H), 2.78-2.60 (m, 2H), 1.78 (m, 3H), 1.75-1.53 (m, 6H), 1.38 (s, 9H), 1.40-1.22 (m, 5H).

Step 2: tert-Butyl 10-((4-(2-methoxyphenyl)-6-oxo-3,6-dihydropyridin-1(2H)-yl)methyl)-7-azaspiro[4.5]decane-7-carboxylate: To a stirred solution of tert-butyl 10-((6-oxo-4-(((trifluoromethyl)sulfonyl)oxy)-3,6-dihydropyridin-1(2H)-yl)methyl)-7-azaspiro[4.5]decane-7-carboxylate (0.50 g, 1.00 mmol) in 3:1 1,4-dioxane/water (40 mL) under an atmosphere of argon were added (2-methoxyphenyl)boronic acid (0.141 g, 1.10 mmol), Pd(dppf)Cl₂·DCM (0.041 g, 0.05 mmol) and sodium carbonate (0.32 g, 3.00 mmol). The reaction mixture was heated to reflux. After 24 h, the organic phase was separated and evaporated to dryness. The crude product was purified by reversed phase preparative HPLC (C18 column) to give the title compound (0.22 g, 57%. LCMS (Method F): R_{T}=1.60 min, m/z=355 [M−Boc+H]⁺. ¹H NMR (400 MHz, CDCl₃): δ 7.28 (t, 1H), 7.22 (d, 1H), 6.99 (t, 1H), 6.87 (d, 1H), 6.12 (s, 1H), 4.00-3.48 (m, 5H), 3.78 (s, 3H), 2.90-2.61 (m, 4H), 1.75-1.48 (m, 8H), 1.42 (s, 9H), 1.38-1.21 (m, 3H).

### Intermediate 37: tert-Butyl (R)-10-((2-oxopyrimidin-1(2H)-yl)methyl)-7-azaspiro[4.5]decane-7-carboxylate

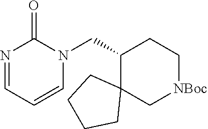 [SMILES: CC(C)(C)OC(=O)N1CC[C@@H](Cn2cccnc2=O)C2(CCCC2)C1]

The title compound was prepared similarly to tert-butyl (R)-10-((6-oxo-4-(o-tolyl)pyrimidin-1(6H)-yl)methyl)-7-azaspiro[4.5]decane-7-carboxylate (Intermediate 18) except using pyrimidin-2(1H)-one (1.25 g, 13 mmol) [commercially available] instead of 6-(o-tolyl)pyrimidin-4(3H)-one in the first step. tert-Butyl 10-((2-oxopyrimidin-1(2H)-yl)methyl)-7-azaspiro[4.5]decane-7-carboxylate (0.41 g) was resolved into the single stereoisomers by chiral HPLC using a Chiralpak IA-3 (20 mm×250 mm, 5 μm) column with isocratic solvent conditions: 80:10:10 hexane/IPA/MeOH (Flow rate: 15 mL/min). The first eluted material (R_{T}=18.84 min) afforded tert-butyl (S)-10-((2-oxopyrimidin-1(2H)-yl)methyl)-7-azaspiro[4.5]decane-7-carboxylate (143 mg). [α]_{D}²⁵=+142.56 (c 0.25 in CHCl₃); and the second eluted material (R_{T}=25.30 min) afforded the title compound (147 mg). LCMS (Method F): R_{T}=1.26 min, m/z=292 [M−butene+H]⁺. ¹H NMR (400 MHz, CDCl₃): δ 8.60 (m, 1H), 7.61 (m, 1H), 6.32 (m, 1H), 4.25 (m, 1H), 4.12 (m, 1H), 4.02 (m, 1H), 3.72 (m, 1H), 2.82-2.65 (m, 2H), 2.01 (m, 2H), 1.65 (m, 3H), 1.53 (m, 2H), 1.38 (s, 9H), 1.40-1.15 (m, 5H). [α]_{D}²⁵=−130.68 (c 0.25 in CHCl₃).

### Intermediate 38: tert-Butyl (R)-10-((6-oxopyridazin-1(6H)-yl)methyl)-7-azaspiro[4.5]decane-7-carboxylate

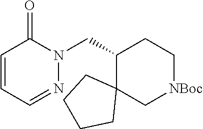 [SMILES: CC(C)(C)OC(=O)N1CC[C@@H](Cn2ncccc2=O)C2(CCCC2)C1]

The title compound was prepared similarly to tert-butyl (R)-10-((6-oxo-4-(o-tolyl)pyrimidin-1(6H)-yl)methyl)-7-azaspiro[4.5]decane-7-carboxylate (Intermediate 18) except using pyridazin-3(2H)-one (2.5 g, 26 mmol) [commercially available] instead of 6-(o-tolyl)pyrimidin-4(3H)-one in the first step. tert-Butyl 10-((6-oxopyridazin-1(6H)-yl)methyl)-7-azaspiro[4.5]decane-7-carboxylate (2.60 g) was resolved into the single stereoisomers by chiral HPLC using a Chiralpak IC (4.6 mm×250 mm, 5 μm) column with isocratic solvent conditions: 70:15:15 hexane/IPA/MeOH (Flow rate: 0.6 mL/min). The first eluted material (R_{T}=20.28 min) afforded the title compound (1.22 g). LCMS (Method F): R_{T}=1.46 min, m/z=248 [M−Boc+H]⁺. ¹H NMR (400 MHz, CDCl₃): δ 7.75 (d, 1H), 7.16 (m, 1H), 6.92 (d, 1H), 4.07 (m, 1H), 3.79 (m, 1H), 3.67 (m, 1H), 3.52 (m, 1H), 2.82-2.65 (m, 2H), 2.01 (m, 1H), 1.65 (m, 3H), 1.53 (m, 2H), 1.38 (s, 9H), 1.40-1.15 (m, 5H). [α]_{D}²⁵=−26.2 (c 0.25 in CHCl₃); and the second eluted material (R_{T}=24.17 min) afforded tert-butyl (S)-10-((6-oxopyridazin-1(6H)-yl)methyl)-7-azaspiro[4.5]decane-7-carboxylate (1.32 g). [α]_{D}²⁵=+26.5 (c 0.25 in CHCl₃).

### Intermediate 39: tert-Butyl (R)-10-((4-(difluoromethyl)-6-oxopyrimidin-1(6H)-yl)methyl)-7-azaspiro[4.5]decane-7-carboxylate

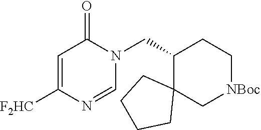 [SMILES: CC(C)(C)OC(=O)N1CC[C@@H](Cn2cnc(C(F)F)cc2=O)C2(CCCC2)C1]

The title compound was prepared similarly to tert-butyl (R)-10-((6-oxo-4-(o-tolyl)pyrimidin-1(6H)-yl)methyl)-7-azaspiro[4.5]decane-7-carboxylate (Intermediate 18) except using 6-(difluoromethyl)pyrimidin-4(3H)-one (0.38 g, 2.6 mmol) [commercially available] instead of 6-(o-tolyl)pyrimidin-4(3H)-one in the first step. tert-Butyl 10-((4-(difluoromethyl)-6-oxopyrimidin-1(6H)-yl)methyl)-7-azaspiro[4.5]decane-7-carboxylate (0.9 g) was resolved into the single stereoisomers by chiral HPLC using a Chiralpak IC (20 mm×250 mm, 5 μm) column with isocratic solvent conditions: 70:15:15 hexane/IPA/MeOH (Flow rate: 12 mL/min). The first eluted material (R_{T}=15.03 min) afforded tert-butyl (S)-10-((4-(difluoromethyl)-6-oxopyrimidin-1(6H)-yl)methyl)-7-azaspiro[4.5]decane-7-carboxylate (0.396 g). [α]_{D}²⁵=+65.16 (c 0.25 in CHCl₃); and the second eluted material (R_{T}=25.28 min) afforded the title compound (0.379 g). LCMS (Method F): R_{T}=4.04 min, m/z=342 [M−butene+H]⁺. ¹H NMR (400 MHz, DMSO-d₆) δ: 8.59 (s, 1H), 6.75 (m, 1H), 6.67 (s, 1H), 4.07 (m, 1H), 3.79 (m, 1H), 3.67 (m, 1H), 3.52 (m, 1H), 2.82-2.55 (m, 2H), 1.80 (m, 2H), 1.65 (m, 2H), 1.53 (m, 2H), 1.38 (s, 9H), 1.40-1.15 (m, 5H). [α]_{D}²⁵=−67.52 (c 0.25 in CHCl₃).

### Intermediate 40: N-((2S,4R)-2-(2,3-Difluorophenyl)piperidin-4-yl)-2,2,2-trifluoro-N-methylacetamide hydrochloride

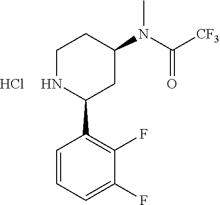 [SMILES: Cl ; CN(C(=O)C(F)(F)F)[C@@H]1CCN[C@H](c2cccc(F)c2F)C1]

The title compound was prepared similarly to N-((2S,4R)-2-(2,5-difluorophenyl)piperidin-4-yl)-2,2,2-trifluoro-N-methylacetamide hydrochloride (Intermediate 19) except using (S)-3-amino-3-(2,3-difluorophenyl)propanoic acid instead of (S)-3-amino-3-(2,5-difluorophenyl)propanoic acid and the diastereoisomers in the analogous Step 8 were separated using flash chromatography instead of chiral preparative HPLC methods. LCMS (Method H): R_{T}=1.82 min, m/z=323 [M+H]⁺. ¹H NMR at 100° C. (400 MHz, DMSO-d₆): δ 9.63 (br s, 1H), 7.77 (s, 1H), 7.49-7.42 (m, 1H), 7.33-7.32 (m, 1H), 4.80-4.77 (d, 1H), 4.69 (m, 1H), 3.50-3.47 (m, 1H), 3.39-3.33 (t, 1H), 3.03 (s, 3H), 2.50-2.46 (m, 2H), 2.03-2.00 (d, 1H), 1.90-1.87 (d, 1H), 1.27 (1H), 0.87 (m, 1H).

### Intermediate 41: N-((2S,4R)-2-(3,4-Difluorophenyl)piperidin-4-yl)-2,2,2-trifluoro-N-methylacetamide hydrochloride

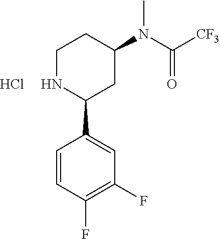 [SMILES: Cl ; CN(C(=O)C(F)(F)F)[C@@H]1CCN[C@H](c2ccc(F)c(F)c2)C1]

The title compound was prepared similarly to N-((2S,4R)-2-(2,5-difluorophenyl)piperidin-4-yl)-2,2,2-trifluoro-N-methylacetamide hydrochloride (Intermediate 19) except using (S)-3-amino-3-(3,4-difluorophenyl)propanoic acid instead of (S)-3-amino-3-(2,5-difluorophenyl)propanoic acid and the diastereoisomers in the analogous Step 8 were separated using the following conditions: Chiralpak IG (21 mm×250 mm, 5 μm) column with isocratic solvent conditions: 90:10 hexane/EtOH+0.1% isopropylamine (Flow rate: 21 mL/min). LCMS (Method C): R_{T}=1.75 min, m/z=323 [M+H]⁺. ¹H NMR at 100° C. (400 MHz, DMSO-d₆): δ 9.49 (br s, 2H), 7.83-7.78 (m, 1H), 7.50-7.43 (m, 2H), 4.60 (br s, 1H), 4.50 (d, 1H), 3.47 (d, 1H), 3.21 (t, 1H), 3.01 (d, 3H), 2.44 (m, 2H), 2.0 (d, 1H), 1.88 (d, 1H).

### Intermediate 42: N-(2,2-Difluoroethyl)-N-((2S,4R)-2-(2,5-difluorophenyl)piperidin-4-yl)-2,2,2-trifluoroacetamide hydrochloride

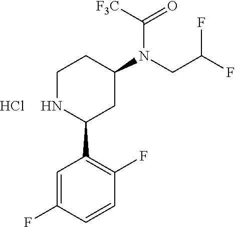 [SMILES: Cl ; O=C(N(CC(F)F)[C@@H]1CCN[C@H](c2cc(F)ccc2F)C1)C(F)(F)F]

Step 1: tert-Butyl (2S)-4-((2,2-difluoroethyl)amino)-2-(2,5-difluorophenyl)piperidine-1-carboxylate: To a stirred solution of tert-butyl (S)-2-(2,5-difluorophenyl)-4-oxopiperidine-1-carboxylate (1.0 g, 3.21 mmol) in MeOH (10 mL) was added 2,2-difluoroethan-1-amine (0.312 mL, 3.85 mmol) at rt. After 8 h, NaBH₃CN (0.606 g, 9.63 mmol) was added. After a further 16 h, the reaction mixture was concentrated under reduced pressure to give the crude product that was purified by flash chromatography (0-10% MeOH in DCM) to yield the title compound (700 mg, 58%). LCMS (Method C): R_{T}=3.55 min, m/z=377 [M+H]⁺.

Step 2: tert-Butyl (2S)-4-(N-(2,2-difluoroethyl)-2,2,2-trifluoroacetamido)-2-(2,5-difluorophenyl)piperidine-1-carboxylate: tert-Butyl (2S)-4-((2,2-difluoroethyl)amino)-2-(2,5-difluorophenyl)piperidine-1-carboxylate (3.0 g, 7.97 mmol) was suspended in DCM (25 mL) and stirred at 0° C. Triethylamine (1.65 mL, 12.0 mmol) and trifluoroacetic anhydride (1.66 mL, 12.0 mmol) were added and the temperature was allowed to increase to rt. After 16 h, the reaction mixture was concentrated under reduced pressure to give the crude product that was purified by flash chromatography (0-30% EtOAc in hexane) to yield the title compound (2.4 g, 64%). LCMS (Method C): R_{T}=3.78 min, m/z=473 [M+H]⁺.

Step 3: tert-Butyl (2S,4R)-4-(N-(2,2-difluoroethyl)-2,2,2-trifluoroacetamido)-2-(2,5-difluorophenyl)piperidine-1-carboxylate: tert-Butyl (2S)-4-(N-(2,2-difluoroethyl)-2,2,2-trifluoroacetamido)-2-(2,5-difluorophenyl)piperidine-1-carboxylate was separated into the single stereoisomers by reversed phase preparative HPLC (C18 column) to give the title compound (first eluting isomer: 840 mg). LCMS (Method C): R_{T}=3.75 min, m/z=473 [M+H]⁺. ¹H NMR at 100° C. (400 MHz, DMSO-d₆): δ 7.21-7.06 (m, 3H), 6.31 (t, 1H), 4.96-4.92 (m, 1H), 4.04 (br s, 1H), 3.93-3.85 (m, 3H), 3.69-3.61 (m, 1H), 2.31-2.32 (m, 1H), 2.16-2.13 (m, 1H), 2.11-2.08 (m, 1H), 1.21 (s, 9H); and tert-Butyl (2S,4S)-4-(N-(2,2-difluoroethyl)-2,2,2-trifluoroacetamido)-2-(2,5-difluorophenyl) piperidine-1-carboxylate (second eluting isomer: 300 mg). LCMS (Method C): R_{T}=3.81 min, m/z=473 [M+H]⁺. ¹H NMR at 100° C. (400 MHz, DMSO-d₆): δ 7.26-7.12 (m, 2H), 6.97 (br s, 1H), 6.33-6.03 (dt, 1H), 5.61 (d, 1H), 4.17 (d, 1H), 3.92-3.81 (m, 3H), 3.68 (br s, 1H), 3.25-3.21 (m, 2H), 2.41-2.33 (m, 1H), 2.20-2.16 (m, 1H), 2.02-1.92 (m, 1H), 1.84-1.81 (m, 1H), 1.37 (s, 9H). [Note: the stereoisomers were confirmed by comparison of ¹H NMR data with close analogues].

Step 4: N-(2,2-Difluoroethyl)-N-((2S,4R)-2-(2,5-difluorophenyl)piperidin-4-yl)-2,2,2-trifluoroacetamide hydrochloride: To a stirred suspension of tert-butyl (2S,4R)-4-(N-(2,2-difluoroethyl)-2,2,2-trifluoroacetamido)-2-(2,5-difluorophenyl)piperidine-1-carboxylate (1.8 g, 3.81 mmol) in DCM (25 mL) was added 4 M HCl in 1,4-dioxane (10 mL) at rt. After 3 h, the solvents were removed in vacuo and the remaining residue was triturated with DCM and pentane, followed by lyophilization to yield the title compound (1.4 g, 90%). LCMS (Method H): R_{T}=2.77 min, m/z=373 [M+H]⁺. ¹H NMR at 100° C. (400 MHz, DMSO-d₆): δ 9.4 (br s, 2H), 7.77 (s, 1H), 7.31 (s, 2H), 6.28 (t, 1H), 4.74 (br s, 1H), 4.35 (br s, 1H), 3.92-3.86 (m, 2H), 3.51-3.48 (m, 1H), 3.37-3.28 (m, 1H), 2.01 (dd, 2H).

### Intermediate 43: N-(3,3-Difluorocyclobutyl)-2,2,2-trifluoro-N-((2S,4R)-2-phenylpiperidin-4-yl)acetamide hydrochloride

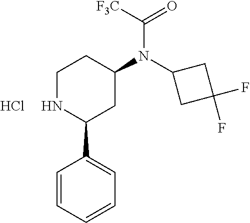 [SMILES: Cl ; O=C(N(C1CC(F)(F)C1)[C@@H]1CCN[C@H](c2ccccc2)C1)C(F)(F)F]

Step 1: tert-Butyl (2S)-4-((3,3-difluorocyclobutyl)amino)-2-phenylpiperidine-1-carboxylate: To a stirred solution of tert-butyl (S)-4-oxo-2-phenylpiperidine-1-carboxylate (1.0 g, 3.63 mmol) in MeOH (10 mL) was added 3,3-difluorocyclobutan-1-amine (1.16 mL, 10.9 mmol) at rt. After 8 h, NaBH₃CN (1.14 g, 18.2 mmol) was added. After a further 16 h, the reaction mixture was concentrated under reduced pressure to give the crude product that was purified by flash chromatography (0-10% MeOH in DCM) to yield the title compound (970 mg, 73%). LCMS (Method C): R_{T}=3.67 min, m/z=367 [M+H]⁺.

Step 2: tert-Butyl (2S)-4-(N-(3,3-difluorocyclobutyl)-2,2,2-trifluoroacetamido)-2-phenylpiperidine-1-carboxylate: tert-Butyl (2S)-4-((3,3-difluorocyclobutyl)amino)-2-phenylpiperidine-1-carboxylate (1.0 g, 2.73 mmol) was suspended in DCM (25 mL) and stirred at 0° C. Triethylamine (2.30 g, 16.4 mmol) and trifluoroacetic anhydride (0.80 mL, 5.74 mmol) were added and the temperature was allowed to increase to rt. After 16 h, the reaction mixture was diluted with water (100 mL) and extracted using DCM (2×100 mL). The organic layer was washed with saturated NaHCO_{3(aq)} solution (50 mL), water (50 mL) and dried (Na₂SO₄). The solvents were evaporated under reduced pressure to give the crude product that was purified by flash chromatography (0-30% EtOAc in hexane) to yield title compound (980 mg, 77%). LCMS (Method C): R_{T}=3.96 min, m/z=463 [M+H]⁺.

Step 3: tert-Butyl (2S,4R)-4-(N-(3,3-difluorocyclobutyl)-2,2,2-trifluoroacetamido)-2-phenylpiperidine-1-carboxylate: tert-Butyl (2S)-4-(N-(3,3-difluorocyclobutyl)-2,2,2-trifluoroacetamido)-2-phenylpiperidine-1-carboxylate was separated into the single stereoisomers by reversed phase preparative HPLC (C18 column) to give the title compound (first eluting isomer: 200 mg). LCMS (Method C): R_{T}=3.94 min, m/z=463 [M+H]⁺. ¹H NMR (400 MHz, DMSO-d₆): δ 7.35-7.22 (m, 5H), 4.78-4.73 (m, 1H), 4.20-4.17 (m, 1H), 3.96-3.91 (m, 2H), 3.59-3.51 (m, 1H), 3.47-3.37 (m, 1H), 3.29-2.93 (m, 1H), 2.91-2.68 (m, 2H), 2.35-2.28 (m, 1H), 2.25-2.08 (m, 1H), 1.96-1.93 (m, 1H), 1.83-1.71 (m, 1H), 1.23-1.14 (m, 9H); and tert-Butyl (2S,4S)-4-(N-(3,3-difluorocyclobutyl)-2,2,2-trifluoroacetamido)-2-phenylpiperidine-1-carboxylate (second eluting isomer: 65 mg). LCMS (Method C): R_{T}=3.99 min, m/z=463 [M+H]⁺. ¹H NMR (400 MHz, DMSO-d₆): δ 7.4-7.37 (m, 2H), 7.29-7.27 (m, 1H), 7.25-7.18 (m, 2H), 5.56-5.47 (m, 1H), 4.23-4.05 (m, 2H), 3.64-3.55 (m, 1H), 3.51-3.43 (m, 2H), 2.75-2.65 (m, 3H), 2.44-2.32 (m, 2H), 1.93-1.91 (m, 1H), 1.56-1.54 (m, 1H), 1.38 (s, 9H). [Note: Stereochemistry was confirmed by nOe data].

Step 3: N-(3,3-Difluorocyclobutyl)-2,2,2-trifluoro-N-((2S,4R)-2-phenylpiperidin-4-yl)acetamide hydrochloride: To a stirred solution of tert-butyl (2S,4R)-4-(N-(3,3-difluorocyclobutyl)-2,2,2-trifluoroacetamido)-2-phenylpiperidine-1-carboxylate (200 mg 0.432 mmol) in DCM (5 mL) was added 4 M HCl in 1,4-dioxane (1 mL) at rt. After 3 h, the solvent was evaporated under reduced pressure and residue was triturated with DCM and pentane followed by lyophilization to yield the title compound (120 mg, 70%). LCMS (Method H): R_{T}=1.93 min. m/z=363 [M+H]⁺. ¹H NMR at 100° C. (400 MHz, DMSO-d₆): δ 9.35 (br s, 1H), 7.63 (d, 2H), 7.47-7.44 (m, 3H), 4.50 (d, 1H), 4.28-4.24 (m, 1H), 4.14 (m, 1H), 3.46-3.24 (m, 4H), 2.85-2.83 (m, 2H), 2.73-2.60 (m, 2H), 2.03 (d, 1H), 1.92 (d, 1H).

### Intermediate 44: 2,2,2-Trifluoro-N-((2S,4R)-2-phenylpiperidin-4-yl)-N-(pyridin-2-ylmethyl)acetamide hydrochloride

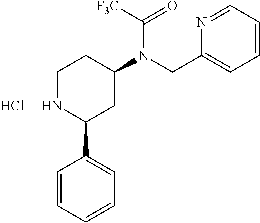 [SMILES: Cl ; O=C(N(Cc1ccccn1)[C@@H]1CCN[C@H](c2ccccc2)C1)C(F)(F)F]

Step 1: tert-Butyl (2S)-2-phenyl-4-((pyridin-2-ylmethyl)amino)piperidine-1-carboxylate: To a stirred solution of tert-butyl (S)-4-oxo-2-phenylpiperidine-1-carboxylate (1.0 g, 3.63 mmol) in MeOH (10 mL) was added pyridin-2-ylmethanamine (436 mg, 4.03 mmol) at rt. After 2 h, NaBH₃CN (914 mg, 14.5 mmol) was added. After a further 16 h, the reaction mixture was diluted with water (100 mL) and extracted with 9:1 DCM/MeOH (2×250 mL). The organic layer was dried (Na₂SO₄) and concentrated under reduced pressure to give the crude product that was purified by flash chromatography (50% EtOAc in hexane) to yield the title compound (1.2 g, 90%). LCMS (Method C): R_{T}=3.27 min, m/z=368 [M+H]⁺.

Step 2: tert-Butyl (2S)-2-phenyl-4-(2,2,2-trifluoro-N-(pyridin-2-ylmethyl)acetamido)piperidine-1-carboxylate: To a stirred solution of tert-butyl (2S)-2-phenyl-4-((pyridin-2-ylmethyl)amino)piperidine-1-carboxylate (1.2 g, 3.26 mmol) in DCM (25 mL) was added triethylamine (1.36 mL, 9.76 mmol) at 0° C. After 10 min, trifluoroacetic anhydride (0.68 mL, 4.89 mmol) was added. The temperature was allowed to increase to rt. After 16 h, the reaction mixture was diluted with water (50 mL) and extracted using DCM (2×50 mL). The organic layer was washed with saturated NaHCO_{3(aq)} solution (100 mL), and dried (Na₂SO₄). The solvents were removed in vacuo to give the crude product that was purified by flash chromatography (30% EtOAc in hexane) to yield the title compound (350 mg, 45%). LCMS (Method C): R_{T}=3.81 min, m/z=464 [M+H]⁺.

Step 3: tert-Butyl (2S,4R)-2-phenyl-4-(2,2,2-trifluoro-N-(pyridin-2-ylmethyl)acetamido)piperidine-1-carboxylate: tert-Butyl (2S)-2-phenyl-4-(2,2,2-trifluoro-N-(pyridin-2-ylmethyl)acetamido)piperidine-1-carboxylate was separated into the single stereoisomers by chiral HPLC using a Chiralpak OD-H (4.6 mm×250 mm, 5 μm) column with isocratic solvent conditions: 90/10 hexane/EtOH+0.1% isopropylamine (Flow rate: 21 mL/min) to give tert-butyl (2S,4S)-2-phenyl-4-(2,2,2-trifluoro-N-(pyridin-2-ylmethyl)acetamido)piperidine-1-carboxylate (first eluting isomer: 120 mg). LCMS (Method C): R_{T}=3.81 min, m/z=464 [M+H]⁺. ¹H NMR at 100° C. (400 MHz, DMSO-d₆): b 8.53 (d, 1H), 7.77 (t. 1H), 7.38-7.23 (m, 5H), 7.17 (d, 2H), 5.47 (br s, 1H), 4.81-4.71 (m, 2H), 4.05-3.99 (m, 2H), 2.78 (br s, 1H), 2.43 (d, 1H), 2.32-2.16 (m, 1H), 1.86-1.76 (m, 1H), 1.67 (d, 1H), 1.42 (m, 9H); and the title compound (second eluting isomer: 250 mg LCMS (Method C): R_{T}=3.81 min, m/z=464 [M+H]⁺. [Note: cis and trans stereochemistry was assigned by comparison of NMR data with analogues].

Step 4: 2,2,2-Trifluoro-N-(1-methylcyclopropyl)-N-((2S,4R)-2-phenylpiperidin-4-yl)acetamide hydrochloride: To a stirred suspension of tert-butyl (2S,4R)-2-phenyl-4-(2,2,2-trifluoro-N-(pyridin-2-ylmethyl)acetamido)piperidine-1-carboxylate (230 mg, 0.496 mmol) in DCM (5 mL) was added 4 M HCl in 1,4-dioxane (2.5 mL) at rt. After 3 h, the solvent was evaporated under reduced pressure and the residue was triturated with DCM and pentane followed by lyophilization to yield the title compound (220 mg, quantitative). LCMS (Method H): R_{T}=1.71 min, m/z=364 [M+H]⁺. ¹H NMR at 100° C. (400 MHz, DMSO-d₆): δ 9.69 (br s, 1H), 9.29 (br s, 1H), 8.52 (s, 1H), 7.80 (t, 1H), 7.64-7.57 (m, 3H), 7.42-7.28 (m, 5H), 4.83 (s, 2H), 4.49-4.46 (m, 2H), 3.42-3.20 (m, 3H), 2.33 (br s, 2H), 2.06 (d, 1H), 1.95 (d, 1H).

### Intermediate 45: 2,2,2-Trifluoro-N-(1-methyl-1H-pyrazol-3-yl)-N-((2S,4R)-2-phenylpiperidin-4-yl)acetamide hydrochloride

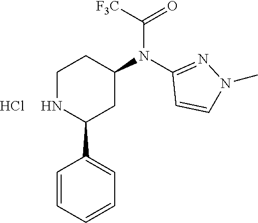 [SMILES: Cl ; Cn1ccc(N(C(=O)C(F)(F)F)[C@@H]2CCN[C@H](c3ccccc3)C2)n1]

Step 1: tert-Butyl (2S)-4-((1-methyl-1H-pyrazol-3-yl)amino)-2-phenylpiperidine-1-carboxylate: To a stirred solution of tert-butyl (S)-4-oxo-2-phenylpiperidine-1-carboxylate (500 mg, 1.82 mmol) in MeOH (5 mL) was added 1-methyl-1H-pyrazol-3-amine (530 mg, 5.46 mmol) and catalytic AcOH (1-2 drops) at rt. After 1 h, NaBH₃CN (571 mg, 9.10 mmol) was added portionwise. After a further 16 h, the reaction mixture was quenched with water (10 mL) and concentrated under reduced pressure to remove the volatiles. The reaction mixture was diluted with water (25 mL) and extracted using ethyl acetate (2×50 mL). The combined organic phase was dried (Na₂SO₄) and concentrated to give the crude product that was purified by flash chromatography (70-80% EtOAc in hexane) to yield the title compound (450 mg, 69%). LCMS (Method C): R_{T}=3.43 min, m/z=357 [M+H]⁺.

Step 2: tert-Butyl (2S,4R)-4-((1-methyl-1H-pyrazol-3-yl)amino)-2-phenylpiperidine-1-carboxylate: tert-Butyl (2S)-4-((1-methyl-1H-pyrazol-3-yl)amino)-2-phenylpiperidine-1-carboxylate (900 mg) was separated into the single stereoisomers by reversed phased preparative HPLC (C18 column) to give the title compound (first eluting isomer: 480 mg). LCMS (Method C): R_{T}=3.41 min, m/z=357 [M+H]⁺. ¹H NMR (400 MHz, DMSO-d₆): δ 7.32-7.26 (m, 3H), 7.21-7.17 (m, 3H), 5.33 (d, 1H), 4.82-4.78 (m, 1H), 4.65 (d, 1H), 3.87-3.82 (m, 1H), 3.57 (s, 3H), 3.51-3.49 (m, 1H), 3.37-3.34 (m, 1H), 2.17-2.13 (m, 1H), 2.05-2.02 (m, 1H), 1.84-1.81 (m, 1H), 1.51-1.47 (m, 1H), 1.22 (s, 9H); and tert-butyl (2S,4S)-4-((1-methyl-1H-pyrazol-3-yl)amino)-2-phenylpiperidine-1-carboxylate (second eluting isomer: 120 mg LCMS (Method C): R_{T}=3.46 min, m/z=357 [M+H]⁺. ¹H NMR (400 MHz, DMSO-d₆): δ 7.39 (t, 2H), 7.28-7.26 (m, 4H), 5.40 (bs, 1H), 5.30 (s, 1H), 4.97 (d, 1H), 3.98 (d, 1H), 3.57 (s, 3H), 2.74-2.63 (m, 2H), 1.88 (d, 1H), 1.62-1.54 (m, 1H), 1.41 (s, 9H). [Note: Stereochemistry was confirmed by nOe data].

Step 3: tert-Butyl (2S,4R)-2-phenyl-4-(2,2,2-trifluoro-N-(1-methyl-1H-pyrazol-3-yl)acetamido)piperidine-1-carboxylate: To a stirred solution of tert-butyl (2S,4R)-4-((1-methyl-1H-pyrazol-3-yl)amino)-2-phenylpiperidine-1-carboxylate (432 mg, 1.21 mmol) in DCM (5 mL) was added triethylamine (1.02 mL, 7.28 mmol), followed by trifluoroacetic anhydride (0.36 mL, 2.55 mmol) at 0° C. The temperature was allowed to increase to rt. After 16 h, the reaction mixture was diluted with water (30 mL) and extracted with DCM (2×75 mL). The combined organic phase was dried (Na₂SO₄) and concentrated to give the crude product that was purified by flash chromatography (0-30% EtOAc in hexane) to yield the title compound (350 mg, 64%). LCMS (Method C): R_{T}=3.67 min, m/z=453 [M+H]⁺.

Step 4: 2,2,2-Trifluoro-N-(1-methyl-1H-pyrazol-3-yl)-N-((2S,4R)-2-phenylpiperidin-4-yl)acetamide hydrochloride: To a stirred solution of tert-butyl (2S,4R)-2-phenyl-4-(2,2,2-trifluoro-N-(1-methyl-1H-pyrazol-3-yl)acetamido)piperidine-1-carboxylate (325 mg, 1.07 mmol) in DCM (5 mL) was added 4 M HCl in 1,4-dioxane (0.2 mL) at 0° C. at rt. After 3 h, the reaction mixture was concentrated under reduced pressure to give the crude product that was triturated with diethyl ether and pentane to yield the title compound (280 mg, 71%). LCMS (Method H): R_{T}=1.65 min, m/z=353 [M+H]⁺. ¹H NMR (400 MHz, DMSO-d₆): δ 9.47 (br s, 2H), 7.81 (d, 1H), 7.51 (d, 2H), 7.44-7.37 (m, 3H), 6.33 (d, 1H), 4.82 (t, 1H), 4.51 (t, 1H), 3.84 (s, 3H), 3.35 (br s, 1H), 3.26-3.20 (m, 1H), 2.10-1.96 (m, 2H), 1.90-1.79 (m, 2H).

### Intermediate 46: 2,2,2-Trifluoro-N-((5-fluoropyridin-2-yl)methyl)-N-((2S,4R)-2-phenylpiperidin-4-yl)acetamide hydrochloride

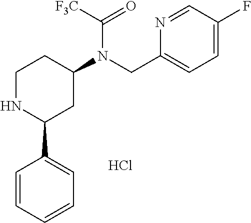 [SMILES: O=C(N(Cc1ccc(F)cn1)[C@@H]1CCN[C@H](c2ccccc2)C1)C(F)(F)F ; Cl]

The title compound was prepared according to the procedure for 2,2,2-trifluoro-N-((2S,4R)-2-phenylpiperidin-4-yl)-N-(pyridin-2-ylmethyl)acetamide hydrochloride (Intermediate 44) except reacting (5-fluoropyridin-2-yl)methanamine (330 mg, 2.61 mmol) instead of pyridin-2-ylmethanamine with tert-butyl-4-oxo-2-phenylpiperidine-1-carboxylate (700 mg, 2.55 mmol) and the following conditions for the separation of diastereoisomers: Chiralpak IG (4.6 mm×250 mm, 5 μm) column with isocratic solvent conditions: 80:20 hexane/EtOH+0.1% isopropylamine (Flow rate: 21 mL/min). LCMS (Method H): R_{T}=1.74 min, m/z=382 [M+H]⁺. ¹H NMR at 100° C. (400 MHz, DMSO-d₆): δ 8.45 (s, 1H), 7.64 (br s, 1H), 7.39-7.19 (m, 5H), 4.75 (s, 2H), 4.14 (br s, 1H), 3.63 (d, 1H), 3.13 (d, 1H), 2.71 (t, 1H), 2.19 (br s, 1H), 1.8-1.68 (m, 4H).

### Intermediate 47: 2,2,2-Trifluoro-N-phenyl-N-((2S,4R)-2-phenylpiperidin-4-yl)acetamide hydrochloride

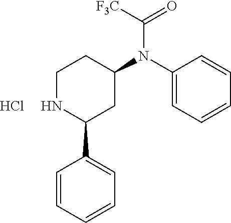 [SMILES: Cl ; O=C(N(c1ccccc1)[C@@H]1CCN[C@H](c2ccccc2)C1)C(F)(F)F]

The title compound was prepared according to the procedure for 2,2,2-trifluoro-N-(1-methyl-1H-pyrazol-3-yl)-N-((2S,4R)-2-phenylpiperidin-4-yl)acetamide hydrochloride (Intermediate 45) except using aniline (0.246 mL, 2.72 mmol) instead of 1-methyl-1H-pyrazol-3-amine with tert-butyl-4-oxo-2-phenylpiperidine-1-carboxylate (500 mg, 1.81 mmol) and the following conditions for the separation of diastereoisomers: Chiralpak IG (21 mm×250 mm, 5 μm) column with isocratic solvent conditions: 90:10 hexane/EtOH+0.1% isopropylamine (Flow rate: 21 mL/min). LCMS (Method I): R_{T}=1.57 min. m/z=349 [M+H]⁺. ¹H NMR at 100° C. (400 MHz, DMSO-d₆): δ9.20 (br s, 1H), 7.51-7.39 (m, 10H), 4.85 (t, 1H), 4.49 (d, 1H) 3.42-3.39 (m, 1H), 3.29-3.22 (m, 1H), 2.32-2.26 (m, 1H), 2.11 (d, 1H) 1.94-1.83 (m, 2H).

### Intermediate 48: 2,2,2-Trifluoro-N-(1-methylcyclopropyl)-N-((2S,4R)-2-phenylpiperidin-4-yl)acetamide hydrochloride

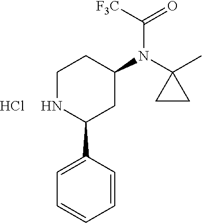 [SMILES: Cl ; CC1(N(C(=O)C(F)(F)F)[C@@H]2CCN[C@H](c3ccccc3)C2)CC1]

Step 1: tert-Butyl (2S)-4-((1-methylcyclopropyl)amino)-2-phenylpiperidine-1-carboxylate: To a stirred solution of tert-butyl (S)-4-oxo-2-phenylpiperidine-1-carboxylate (1.0 g, 3.63 mmol) in MeOH (50 mL) was added 1-methylcyclopropan-1-amine hydrochloride (584 mg, 5.44 mmol) at rt. After 2 h, NaBH₃CN (684 mg, 10.9 mmol) was added. After a further 16 h, the reaction mixture was diluted with water (100 mL) and extracted using 5% (v/v) MeOH in DCM (2×200 mL). The combined organic phase was dried (Na₂SO₄) and concentrated under reduced pressure to give the crude product that was purified by flash chromatography (50% EtOAc in hexane) to yield the title compound (600 mg, 50%). LCMS (Method C): R_{T}=3.62 min, m/z=331 [M+H]⁺.

Step 2: tert-Butyl (2S)-2-phenyl-4-(2,2,2-trifluoro-N-(1-methylcyclopropyl)acetamido)piperidine-1-carboxylate: To a stirred solution of tert-butyl (2S)-4-((1-methylcyclopropyl)amino)-2-phenylpiperidine-1-carboxylate (600 mg, 1.81 mmol) in DCM (5 mL) was added triethylamine (0.5 mL, 3.63 mmol) at 0° C. After 10 min, trifluoroacetic anhydride (0.38 mL, 2.71 mmol) was added and the temperature was allowed to increase to rt. After a further 16 h, the reaction mixture was diluted with water (50 mL) and extracted using DCM (2×50 mL). The combined organic phase was washed using saturated NaHCO_{3(aq)} solution (100 mL), and dried (Na₂SO₄). The solvents were evaporated under reduced pressure to give the crude product that was purified by flash chromatography to yield the title compound (350 mg, 45%). LCMS (Method C): R_{T}=3.84 min, m/z=427 [M+H]⁺.

Step 3: tert-Butyl (2S,4R)-2-phenyl-4-(2,2,2-trifluoro-N-(1-methycyclopropyl)acetamido)piperidine-1-carboxylate: tert-Butyl (2S)-2-phenyl-4-(2,2,2-trifluoro-N-(1-methylcyclopropyl)acetamido)piperidine-1-carboxylate was separated into the single stereoisomers chiral HPLC using a Chiralpak IC (4.6 mm×250 mm, 5 μm) column with isocratic solvent conditions: 90/10 hexane/EtOH+0.1% isopropylamine (Flow rate: 21 mL/min) to give the title compound (first eluting isomer: 150 mg). LCMS (Method C): R_{T}=3.84 min, m/z=427 [M+H]⁺. ¹H NMR at 100° C. (400 MHz, DMSO-d₆): δ 7.33-7.29 (m, 2H), 7.25-7.19 (m, 3H), 4.78-4.74 (m, 1H), 4.01-3.97 (m, 1H), 3.89-3.87 (m, 1H), 3.54-3.46 (m, 1H), 2.56-2.53 (m, 1H), 2.15 (br s, 1H), 1.95-1.92 (m, 2H), 1.42 (s, 3H), 1.22 (s, 9H), 1.17-1.06 (br s, 2H), 0.83 (br s, 2H); and tert-butyl (2S,4S)-2-phenyl-4-(2,2,2-trifluoro-N-(1-methylcyclopropyl)acetamido)piperidine-1-carboxylate (second eluting isomer: 130 mg). LCMS (Method C): R_{T}=3.85 min, m/z=427 [M+H]⁺. ¹H NMR (400 MHz, DMSO-d₆): δ 7.43-7.39 (m, 2H), 7.29-7.23 (m, 3H), 5.66 (s, 0.35H), 5.49 (s, 1H), 4.09-4.05 (m, 1H), 3.63-3.57 (m, 1H), 2.92-2.85 (m, 1H), 2.67-2.64 (m, 1H), 2.43-2.40 (m, 1H), 2.32-2.26 (m, 1H), 1.72-1.69 (d, 1H), 1.44 (s, 9H), 1.35 (s, 3H), 1.08 (br s, 2H), 0.85-0.74 (br s, 2H). [Note: cis and trans stereochemistry was assigned by comparison of NMR data with analogues].

Step 3: 2,2,2-Trifluoro-N-(1-methylcyclopropyl)-N-((2S,4R)-2-phenylpiperidin-4-yl)acetamide hydrochloride: To a stirred suspension of tert-butyl (2S,4R)-2-phenyl-4-(2,2,2-trifluoro-N-(1-methylcyclopropyl)acetamido)piperidine-1-carboxylate (150 mg, 0.351 mmol) in DCM (5 mL) was added 4 M HCl in 1,4-dioxane (1 mL) at rt. After 3 h, the solvents were evaporated under reduced pressure and residue was triturated with DCM and pentane followed by lyophilization to yield the title compound (89 mg, 70%). LCMS (Method C): R_{T}=2.49 min, m/z=327 [M+H]⁺. ¹H NMR at 100° C. (400 MHz, DMSO-d₆): δ 9.30 (br s, 1H), 7.58-7.56 (m, 2H), 7.47-7.41 (m, 3H), 4.43-4.40 (d, 1H), 4.11-4.06 (m, 1H), 3.43-3.40 (m, 1H), 3.24-3.18 (m, 1H), 2.84-2.72 (m, 2H), 2.08 (d, 1H), 1.97 (d, 1H), 1.44 (s, 3H), 1.20 (s, 2H), 0.84 (br s, 2H).

### Intermediate 49: N-((2S,4R)-2-(2,5-Difluorophenyl)piperidin-4-yl)-N-ethyl-2,2,2-trifluoroacetamide hydrochloride

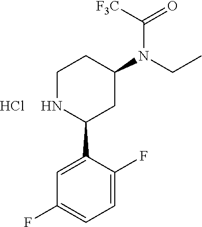 [SMILES: Cl ; CCN(C(=O)C(F)(F)F)[C@@H]1CCN[C@H](c2cc(F)ccc2F)C1]

Step 1: tert-Butyl (2S)-2-(2,5-difluorophenyl)-4-(ethylamino)piperidine-1-carboxylate: To a stirred solution of tert-butyl (S)-2-(2,5-difluorophenyl)-4-oxopiperidine-1-carboxylate (700 mg, 2.41 mmol) in MeOH (5 mL) was added ethylamine (163 mg, 3.61 mmol) at rt. After 2 h, NaBH₃CN (454 mg, 7.23 mmol) was added. After a further 16 h, the reaction mixture was concentrated under reduced pressure to give the crude product that was purified by flash chromatography (0-10% MeOH in DCM) to yield title compound (820 mg, 91%). LCMS (Method C): R_{T}=2.94 min, m/z=341 [M+H]⁺.

Step 2: tert-Butyl (2S)-2-(2,5-difluorophenyl)-4-(N-ethyl-2,2,2-trifluoroacetamido)piperidine-1-carboxylate: tert-Butyl (2S)-2-(2,5-difluorophenyl)-4-(ethylamino)piperidine-1-carboxylate (700 mg, 2.06 mmol) was suspended in DCM (5 mL) and stirred at 0° C. Pyridine (1.63 mg, 20.6 mmol) and trifluoroacetic anhydride (648 mg, 3.08 mmol) were added and the temperature was allowed to increase to rt. After 16 h, the reaction mixture was concentrated under reduced pressure to give the crude product that was purified by flash chromatography (0-30% EtOAc in hexane) to yield the title compound (960 mg, 77%). LCMS (Method C): R_{T}=3.79 min, m/z=437 [M+H]⁺.

Step 3: tert-Butyl (2S,4R)-2-(2,5-difluorophenyl)-4-(N-ethyl-2,2,2-trifluoroacetamido)piperidine-1-carboxylate: tert-Butyl (2S)-2-(2,5-difluorophenyl)-4-(N-ethyl-2,2,2-trifluoroacetamido)piperidine-1-carboxylate was separated into the single stereoisomers by reversed phased preparative HPLC (C18 column) to give the title compound (first eluting isomer: 250 mg). LCMS (Method C): R_{T}=3.79 min, m/z=437 [M+H]⁺. ¹H NMR at 100° C. (400 MHz, DMSO-d₆): δ 7.20-7.08 (m, 3H), 4.95-4.90 (m, 1H), 3.98-3.89 (m, 2H), 3.68-3.61 (m, 1H), 3.44 (s, 2H), 2.16-2.06 (m, 1H), 1.98-1.90 (m, 2H), 1.90-1.07 (d, 12H); and tert-butyl (2S,4S)-2-(2,5-difluorophenyl)-4-(N-ethyl-2,2,2-trifluoroacetamido)piperidine-1-carboxylate (second eluting isomer: 100 mg). LCMS (Method C): R_{T}=3.80 min, m/z=437 [M+H]⁺. ¹H NMR at 100° C. (400 MHz, DMSO-d₆): δ 7.26-7.20 (m, 1H), 7.17-7.13 (m, 1H), 7.00 (br s, 1H), 5.62-5.60 (d, 1H), 4.18-4.18 (d, 1H), 3.89 (br s, 1H), 3.44-3.39 (m, 2H), 3.19 (t, 1H), 2.50-2.32 (m, 1H), 2.18-2.14 (m, 1H), 2.01-1.93 (m, 1H), 1.84-1.81 (m, 1H), 1.37 (s, 9H), 1.16 (m, 3H). [Note: cis and trans stereochemistry confirmed by nOe data].

Step 3: N-((2S,4R)-2-(2,5-Difluorophenyl)piperidin-4-yl)-N-ethyl-2,2,2-trifluoroacetamide hydrochloride: To a stirred suspension of tert-butyl (2S,4R)-2-(2,5-difluorophenyl)-4-(N-ethyl-2,2,2-trifluoroacetamido)piperidine-1-carboxylate (290 mg 0.664 mmol) in DCM (5 mL) was added 4 M HCl in 1,4-dioxane (2 mL) at rt. After 3 h, the solvents were evaporated under reduced pressure and residue was triturated with DCM and pentane followed by lyophilization to yield the title compound (232 mg, 97%). LCMS (Method H): R_{T}=2.64 min, m/z=337 [M+H]⁺. ¹H NMR at 100° C. (400 MHz, DMSO-d₆): δ 9.81 (br s, 2H), 7.91 (s, 1H), 7.35-7.28 (m, 2H), 4.73-4.70 (m, 1H), 4.38-4.32 (m, 1H), 3.51-3.46 (m, 3H), 3.36-3.30 (m, 1H), 2.67-2.63 (m, 2H), 2.05-1.92 (dd, 2H), 1.22 (s, 3H).

### Intermediate 50: N-((2S,4R)-2-(2,5-Difluorophenyl)piperidin-4-yl)-2,2,2-trifluoro-N-isopropylacetamide hydrochloride

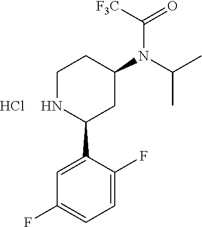 [SMILES: Cl ; CC(C)N(C(=O)C(F)(F)F)[C@@H]1CCN[C@H](c2cc(F)ccc2F)C1]

The title compound was prepared according to the procedure for N-((2S,4R)-2-(2,5-difluorophenyl)piperidin-4-yl)-N-ethyl-2,2,2-trifluoroacetamide hydrochloride (Intermediate 49) except isopropylamine (171 mg, 2.89 mmol) was used instead of ethylamine with tert-butyl (S)-2-(2,5-difluorophenyl)-4-oxopiperidine-1-carboxylate (600 mg, 1.93 mmol) and reversed phase preparative HPLC (C18 column) was used for the separation of diastereoisomers. LCMS (Method C): R_{T}=1.75 min, m/z=255 [M+H]⁺. ¹H NMR (400 MHz, DMSO-d₆): δ 10.24 (br s, 1H), 9.75 (br s, 1H), 9.24 (br s, 2H), 7.85 (s, 1H), 7.44-7.34 (m, 2H), 4.68 (t, 1H), 3.72 (m, 1H), 3.56-3.43 (m, 2H), 3.22 (m, 1H), 2.38-2.35 (m, 1H), 2.27-2.24 (m, 1H), 2.16-2.07 (m, 2H), 1.27 (d, 6H).

### Intermediate 51: N-(2-((tert-Butyldimethylsilyl)oxy)ethyl)-2,2,2-trifluoro-N-((2S,4R)-2-phenylpiperidin-4-yl)acetamide

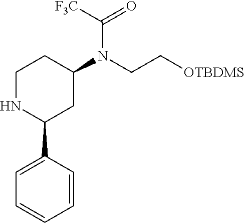 [SMILES: CC(C)(C)[Si](C)(C)OCCN(C(=O)C(F)(F)F)[C@@H]1CCN[C@H](c2ccccc2)C1]

Step 1: tert-Butyl (2S)-4-((2-hydroxyethyl)amino)-2-phenylpiperidine-1-carboxylate: To stirred solution of tert-butyl (S)-4-oxo-2-phenylpiperidine-1-carboxylate (1.00 g, 3.63 mmol) in MeOH (25 mL) were added 2-aminoethan-1-ol (0.32 mL, 5.44 mmol) and catalytic AcOH (1-2 drops) at rt. After 2 h, NaBH₃CN (0.685 g, 10.9 mmol) was added. After a further 16 h, the reaction mixture was diluted with water (50 mL) and extracted with 5% (v/v) MeOH in DCM (3×50 mL). The organic layer was dried (Na₂SO₄) and concentrated under reduced pressure to give the crude product that was purified by flash chromatography (0-10% MeOH in DCM) to yield the title compound (1.00 g, 86%). LCMS (Method C): R_{T}=2.79 min, m/z=321 [M+H]⁺.

Step 2: tert-Butyl (2S)-4-((2-((tert-butyldimethylsilyl)oxy)ethyl)amino)-2-phenylpiperidine-1-carboxylate: To a stirred solution of tert-butyl (2S)-4-((2-hydroxyethyl)amino)-2-phenylpiperidine-1-carboxylate (1.00 g, 3.12 mmol) in DCM (25 mL) were added TBDMSCI (940 mg, 6.24 mmol) and imidazole (1.06 g, 15.6 mmol) at rt. After 16 h, the reaction mixture was diluted with water (100 mL) and extracted with EtOAc (2×100 mL). The combined organic phase was washed with saturated NaHCO_{3(aq)} solution (100 ml) and dried (Na₂SO₄). The solvents were evaporated under reduced pressure to give the crude product that was purified by flash chromatography to yield the title compound (750 mg, 55%). LCMS (Method C): R_{T}=2.51 min, m/z=435 [M+H]⁺.

Step 3: tert-Butyl (2S)-4-(N-(2-((tert-butyldimethylsilyl)oxy)ethyl)-2,2,2-trifluoroacetamido)-2-phenylpiperidine-1-carboxylate: To a stirred solution of tert-butyl (2S)-4-((2-((tert-butyldimethylsilyl)oxy)ethyl)amino)-2-phenylpiperidine-1-carboxylate (750 mg, 1.73 mmol) in DCM (25 mL) was added pyridine (1.39 mL, 17.3 mmol) at 0° C., followed by trifluoroacetic anhydride (0.362 mL, 2.56 mmol). After 16 h, the reaction mixture was diluted with water (100 mL) and extracted using DCM (2×100 mL). The organic layer was washed with saturated NaHCO_{3(aq)} solution (100 mL) and dried (Na₂SO₄). The solvents were evaporated under reduced pressure to give the crude product that was purified by flash chromatography to yield the title compound (750 mg, 82%). LCMS (Method C): R_{T}=2.81 min, m/z=531 [M+H]⁺.

Step 4: tert-Butyl (2S,4R)-4-(N-(2-((tert-butyldimethylsilyl)oxy)ethyl)-2,2,2-trifluoroacetamido)-2-phenylpiperidine-1-carboxylate: tert-Butyl (2S)-4-(N-(2-((tert-butyldimethylsilyl)oxy)ethyl)-2,2,2-trifluoroacetamido)-2-phenylpiperidine-1-carboxylate was separated by reversed phase preparative HPLC (C18 column) to give the title compound (first eluting isomer: 200 mg). LCMS (Method C): R_{T}=2.81 min, m/z=531 [M+H]⁺. ¹H NMR at 100° C. (400 MHz, DMSO-d₆): δ 7.33-7.30 (m, 2H), 7.25-7.20 (m, 3H), 4.79-4.75 (m, 1H), 3.96-3.92 (m, 1H), 3.78-3.76 (m, 2H), 3.56-3.48 (m, 3H), 2.07-2.04 (m, 1H), 1.94-1.90 (m, 4H), 1.83-1.2 (m, 9H), 0.85 (s, 9H), 0.03 (s, 6H); and tert-butyl (2S,4S)-4-(N-(2-((tert-butyldimethylsilyl)oxy)ethyl)-2,2,2-trifluoroacetamido)-2-phenylpiperidine-1-carboxylate (second eluting isomer: 190 mg). LCMS (Method C): R_{T}=2.79 min, m/z=531 [M+H]⁺. ¹H NMR 100° C. (400 MHz, DMSO-d₆): δ 7.40-7.36 (m, 2H), 7.27-7.23 (m, 3H), 5.52 (s, 1H), 4.12-4.08 (m, 1H), 3.96-3.92 (m, 1H), 3.83-3.82 (m, 1H), 3.75-3.72 (m, 2H), 3.50-3.45 (m, 2H), 2.87-2.80 (m, 1H), 2.49-2.41 (m, 1H), 2.32 (br s, 1H), 1.68-1.64 (d, 1H), 1.60 (s, 9H), 0.85 (s, 9H), 0.06 (s, 6H). [Note: cis and trans stereochemistry was assigned by comparison of NMR data with analogues].

Step 4: N-(2-((tert-Butyldimethylsilyl)oxy)ethyl)-2,2,2-trifluoro-N-((2S,4R)-2-phenylpiperidin-4-yl)acetamide: tert-Butyl (2S,4R)-4-(N-(2-((tert-butyldimethylsilyl)oxy)ethyl)-2,2,2-trifluoroacetamido)-2-phenylpiperidine-1-carboxylate (250 mg, 0.471 mmol) was suspended in 1,1,1,3,3,3-hexafluoropropan-2-ol (5 mL) and heated to 120° C. After 16 h, the solvent was evaporated under reduced pressure and the residue was purified by reversed phase preparative HPLC to yield the title compound (164 mg, 81%). LCMS (Method J): R_{T}=4.00 min, m/z=431 [M+H]⁺. ¹H NMR at 100° C. (400 MHz, DMSO-d₆): δ 7.38-7.36 (m, 2H), 7.32-7.20 (m, 3H), 3.93 (br s, 1H), 3.75-3.72 (m, 2H), 3.66-3.62 (m, 1H), 3.49 (br s, 2H), 3.21-3.16 (m, 1H), 2.76-2.70 (m, 1H), 1.91 (br s, 1H), 1.18-1.67 (m, 4H), 0.85 (s, 9H), 0.21 (s, 1H).

### Intermediate 52: tert-Butyl (R)-10-((4-cyclopropyl-6-oxopyrimidin-1(6H)-yl)methyl)-7-azaspiro[4.5]decane-7-carboxylate

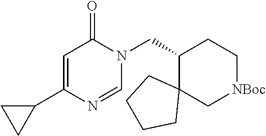 [SMILES: CC(C)(C)OC(=O)N1CC[C@@H](Cn2cnc(C3CC3)cc2=O)C2(CCCC2)C1]

The title compound was prepared similarly to tert-butyl (R)-10-((6-oxo-4-(o-tolyl)pyrimidin-1(6H)-yl)methyl)-7-azaspiro[4.5]decane-7-carboxylate (Intermediate 18) except using 6-cyclopropylpyrimidin-4(3H)-one (0.35 g, 2.6 mmol) [commercially available] instead of 6-(o-tolyl)pyrimidin-4(3H)-one in the first step. tert-Butyl 10-((4-cyclopropyl-6-oxopyrimidin-1(6H)-yl)methyl)-7-azaspiro[4.5]decane-7-carboxylate (0.30 g) was resolved into the single stereoisomers by chiral HPLC using a Chiralpak IA (20 mm×250 mm, 5 μm) column with isocratic solvent conditions: 70:15:15 hexane/IPA/MeOH (Flow rate: 15 mL/min). The first eluted material (R_{T}=12.03 min) afforded tert-butyl (S)-10-((4-(difluoromethyl)-6-oxopyrimidin-1(6H)-yl)methyl)-7-azaspiro[4.5]decane-7-carboxylate (92 mg). [α]_{D}²⁵=+59.6 (c 0.25 in MeOH); and the second eluted material (R_{T}=19.82 min) afforded the title compound (99 mg). LCMS (Method F): R_{T}=4.17 min, m/z=332 [M−butene+H]⁺. ¹H NMR (400 MHz, DMSO-d₆) δ: 8.28 (s, 1H), 6.31 (s, 1H), 4.06 (m, 1H), 3.80 (m, 1H), 3.65 (m, 1H), 3.52 (m, 1H), 2.80-2.54 (m, 2H), 1.85 (m, 1H), 1.80 (m, 2H), 1.65 (m, 2H), 1.53 (m, 2H), 1.39 (s, 9H), 1.40-1.15 (m, 5H), 0.90 (m, 4H). [α]_{D}²⁵=−59.2 (c 0.25 in MeOH).

### Intermediate 53: tert-Butyl 10-((6-oxo-4-phenyl-3,6-dihydropyridin-1(2H)-yl)methyl)-7-azaspiro[4.5]decane-7-carboxylate

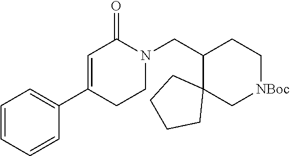 [SMILES: CC(C)(C)OC(=O)N1CCC(CN2CCC(c3ccccc3)=CC2=O)C2(CCCC2)C1]

To a stirred solution of tert-butyl 10-((6-oxo-4-(((trifluoromethyl)sulfonyl)oxy)-3,6-dihydropyridin-1(2H)-yl)methyl)-7-azaspiro[4.5]decane-7-carboxylate (0.50 g, 1.00 mmol) in 3:1 1,4-dioxane/water (40 mL) under argon atmosphere were added phenylboronic acid (0.135 g, 1.10 mmol), Pd(dppf)·Cl₂ (0.041 g, 0.05 mmol) and sodium carbonate (0.32 g, 3.00 mmol). The reaction mixture was degassed, backfilled with argon and brought to reflux. After 24 h, the organic phase was separated and evaporated to dryness. The crude product was purified by reversed phase preparative HPLC (C18 column) to give the title compound (0.32 g, 72%). LCMS (Method F): R_{T}=1.53 min, m/z=369 [M−butene+H]⁺. ¹H NMR (400 MHz, CDCl₃): δ 7.49 (m, 2H), 7.38 (m, 3H), 6.28 (s, 1H), 4.00-3.48 (m, 5H), 2.90-2.61 (m, 4H), 1.75-1.48 (m, 8H), 1.43 (s, 9H), 1.40-1.22 (m, 3H).

### Intermediate 54: tert-Butyl (R)-10-((2-oxo-4-phenyl-2,5-dihydro-1H-pyrrol-1-yl)methyl)-7-azaspiro[4.5]decane-7-carboxylate

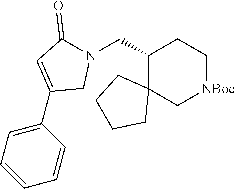 [SMILES: CC(C)(C)OC(=O)N1CC[C@@H](CN2CC(c3ccccc3)=CC2=O)C2(CCCC2)C1]

The title compound was prepared similarly to tert-butyl (R)-10-((6-oxo-4-(o-tolyl)pyrimidin-1(6H)-yl)methyl)-7-azaspiro[4.5]decane-7-carboxylate (Intermediate 18) except using 4-phenyl-1,5-dihydro-2H-pyrrol-2-one (2.00 g, 12.6 mmol) [commercially available] instead of 6-(o-tolyl)pyrimidin-4(3H)-one in the first step. tert-Butyl 10-((2-oxo-4-phenyl-2,5-dihydro-H-pyrrol-1-yl)methyl)-7-azaspiro[4.5]decane-7-carboxylate (1.1 g) was resolved into the single stereoisomers by chiral HPLC using a CHIRALCEL OZ-H (20 mm×250 mm, 5 μm) column with isocratic solvent conditions: 80:10:10 hexane/IPA/MeOH (Flow rate: 20 mL/min). The first eluted material (R_{T}=13.43 min) afforded the title compound (0.43 g). LCMS (Method F): R_{T}=1.49 min, m/z=355 [M−butene+H]⁺. ¹H NMR (500 MHz, CDCl₃) δ: 8.37 (s, 1H), 7.43-7.35 (m, 5H), 6.35 (s, 1H), 4.26-4.15 (m, 2H), 3.74 (m, 1H), 3.54 (m, 1H), 2.68-2.36 (m, 3H), 1.79 (m, 2H), 1.65 (m, 2H), 1.53 (m, 2H), 1.38 (s, 9H), 1.40-1.14 (m, 5H). [α]_{D}²⁵=−9.76 (c 0.25 in CHCl₃); and the second eluted material (R_{T}=22.43 min) afforded tert-butyl (R)-10-((2-oxo-4-phenyl-2,5-dihydro-1H-pyrrol-1-yl)methyl)-7-azaspiro[4.5]decane-7-carboxylate (0.44 g). [α]_{D}²⁵=+10.44 (c 0.25 in CHCl₃).

### Intermediate 55: N-(3,3-Difluorocyclobutyl)-N-((2S,4R)-2-(2,5-difluorophenyl)piperidin-4-yl)-2,2,2-trifluoroacetamide hydrochloride

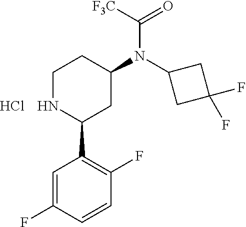 [SMILES: Cl ; O=C(N(C1CC(F)(F)C1)[C@@H]1CCN[C@H](c2cc(F)ccc2F)C1)C(F)(F)F]

The title compound was prepared according to the procedure for N-(3,3-difluorocyclobutyl)-2,2,2-trifluoro-N-((2S,4R)-2-phenylpiperidin-4-yl)acetamide hydrochloride (Intermediate 43) except using tert-butyl (S)-2-(2,5-difluorophenyl)-4-oxopiperidine-1-carboxylate instead of tert-butyl (S)-4-oxo-2-phenylpiperidine-1-carboxylate and the following conditions for the separation of diastereoisomers: Chiralpak IG (21 mm×250 mm, 5 μm) column with isocratic solvent conditions: 98:2 hexane/EtOH+0.1% isopropylamine (Flow rate: 21 mL/min). LCMS (Method H): R_{T}=2.92 min, m/z=399 [M+H]⁺. ¹H NMR at 100° C. (400 MHz, DMSO-d₆): δ 9.44 (br s, 2H), 7.78 (s, 1H), 7.30 (m, 2H), 4.77 (d, 1H), 4.23-4.21 (m, 2H), 3.58-3.34 (m, 4H), 2.85-2.82 (m, 2H), 2.66-2.56 (m, 2H), 2.00 (d, 1H), 1.90 (d, 1H).

### Intermediate 56: 2-(Trimethylsilyl)ethyl ((3R,6S)-6-phenylpiperidin-3-yl)carbamate and 2-(trimethylsilyl)ethyl ((3S,6R)-6-phenylpiperidin-3-yl)carbamate

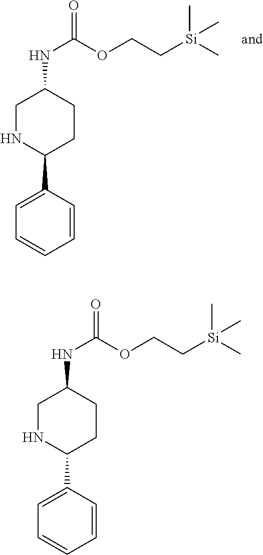 [SMILES: C[Si](C)(C)CCOC(=O)N[C@@H]1CC[C@@H](c2ccccc2)NC1 ; C[Si](C)(C)CCOC(=O)N[C@H]1CC[C@H](c2ccccc2)NC1]

rac-Benzyl (2S,5R)-2-(2,5-difluorophenyl)-5-(((2-(trimethylsilyl)ethoxy)carbonyl)amino)piperidine-1-carboxylate was prepared similarly to rac-benzyl (2S,4R)-2-(2-fluorophenyl)-4-(((2-(trimethylsilyl)ethoxy)carbonyl)amino)piperidine-1-carboxylate (see Intermediate 28) except using methyl 6-bromonicotinate and (2,5-difluorophenyl)boronic acid instead of methyl 2-bromoisonicotinate and (2-fluorophenyl)boronic acid in the initial step. The Teoc group was removed using General Procedure 10 to give the title compound that was confirmed to be trans by 2D NMR analysis. LCMS (Method A): R_{T}=0.65 min, m/z 321 [M+H]⁺. ¹H NMR (500 MHz, DMSO-d₆): δ 7.34 (d, 2H), 7.28 (t, 2H), 7.21 (t, 1H), 6.94 (d, 1H), 4.12-3.98 (m, 2H), 3.43 (d, 1H), 3.40-3.32 (m, 1H), 3.07 (d, 1H), 2.37 (t, 1H), 1.91-1.84 (m, 1H), 1.76-1.70 (m, 1H), 1.43-1.32 (m, 2H), 0.98-0.88 (m, 2H), 0.03 (s, 9H). [1×NH signal not observed].

### Intermediate 57: 2,2,2-Trifluoro-N-(2-fluoroethyl)-N-((2S,4R)-2-(3-fluorophenyl)piperidin-4-yl)acetamide hydrochloride

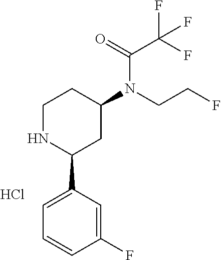 [SMILES: Cl ; O=C(N(CCF)[C@@H]1CCN[C@H](c2cccc(F)c2)C1)C(F)(F)F]

The title compound was prepared according to the procedure for N-(2,2-difluoroethyl)-N-((2S,4R)-2-(2,5-difluorophenyl)piperidin-4-yl)-2,2,2-trifluoroacetamide hydrochloride (Intermediate 42) except using tert-butyl (S)-2-(3-fluorophenyl)-4-oxopiperidine-1-carboxylate instead of tert-butyl (S)-2-(2,5-difluorophenyl)-4-oxopiperidine-1-carboxylate, 2-fluoroethan-1-amine instead of 2,2-difluoroethan-1-amine, and reversed phase preparative HPLC (C18 column) for the separation of diastereoisomers. LCMS (Method C): R_{T}=2.56 min, m/z=337 [M+H]⁺. ¹H NMR at 100° C. (400 MHz, DMSO-d₆): δ 9.59 (br s, 1H), 7.57-7.46 (m, 3H), 7.22 (t, 1H), 4.70 (s, 1H), 4.58 (s, 1H), 4.48 (d, 1H), 4.29-4.23 (m, 1H), 3.82-3.76 (m, 2H), 3.48-3.41 (m, 1H), 3.21 (t, 1H), 2.67-2.56 (m, 2H), 2.09-1.93 (m, 2H).

### Intermediate 58: 2,2,2-Trifluoro-N-((2S,4R)-2-(3-fluorophenyl)piperidin-4-yl)-N-(2-methoxyethyl)acetamide hydrochloride

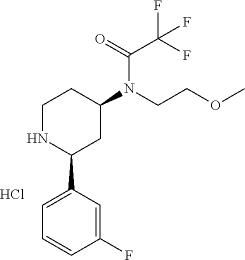 [SMILES: Cl ; COCCN(C(=O)C(F)(F)F)[C@@H]1CCN[C@H](c2cccc(F)c2)C1]

The title compound was prepared according to the procedure for N-(2,2-difluoroethyl)-N-((2S,4R)-2-(2,5-difluorophenyl)piperidin-4-yl)-2,2,2-trifluoroacetamide hydrochloride (Intermediate 42) except using tert-butyl (S)-2-(3-fluorophenyl)-4-oxopiperidine-1-carboxylate instead of tert-butyl (S)-2-(2,5-difluorophenyl)-4-oxopiperidine-1-carboxylate, 2-methoxyethan-1-amine instead of 2,2-difluoroethan-1-amine, and reversed phase preparative HPLC (C18 column) for the separation of diastereoisomers. LCMS (Method C): R_{T}=2.56 min, m/z=349 [M+H]⁺. ¹H NMR at 100° C. (400 MHz, DMSO-d₆): δ 9.50 (br s, 2H), 7.53-7.44 (m, 3H), 7.22 (t, 1H), 4.46 (d, 1H), 4.21 (t, 1H), 3.60-3.55 (m, 4H), 3.48-3.44 (m, 1H), 3.31 (s, 3H), 3.20 (t, 1H), 2.61-2.55 (m, 1H), 2.08-1.92 (m, 2H).

### Intermediate 59: tert-Butyl (R)-10-((6-chloro-4-oxoquinazolin-3(4H)-yl)methyl)-7-azaspiro[4.5]decane-7-carboxylate

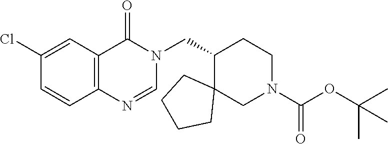 [SMILES: CC(C)(C)OC(=O)N1CC[C@@H](Cn2cnc3ccc(Cl)cc3c2=O)C2(CCCC2)C1]

The title compound was prepared similarly to the procedure for tert-butyl (R)-10-((6-fluoro-4-oxoquinazolin-3(4H)-yl)methyl)-7-azaspiro[4.5]decane-7-carboxylate (Intermediate 27) except using 6-chloroquinazolin-4(3H)-one instead of 6-fluoroquinazolin-4(3H)-one. LCMS (Method B): R_{T}=1.91 min, m/z=432 [M+H]⁺. ¹H NMR (500 MHz, DMSO-d₆): δ 8.40 (s, 1H), 8.09 (d, J=2.5 Hz, 1H), 7.86 (dd, J=8.7, 2.5 Hz, 1H), 7.72 (d, J=8.7 Hz, 1H), 4.17 (dd, J=13.3, 3.0 Hz, 1H), 3.83-3.72 (m, 2H), 3.55 (s, 1H), 2.83-2.65 (m, 1H), 1.92-1.82 (m, 2H), 1.74-1.62 (m, 2H), 1.62-1.50 (m, 2H), 1.49-1.40 (m, 2H), 1.39 (s, 9H), 1.36-1.16 (m, 4H).

### Intermediate 60: tert-Butyl (R)-10-((4-oxoquinazolin-3(4H)-yl)methyl)-7-azaspiro[4.5]decane-7-carboxylate

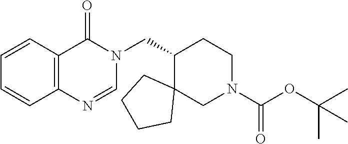 [SMILES: CC(C)(C)OC(=O)N1CC[C@@H](Cn2cnc3ccccc3c2=O)C2(CCCC2)C1]

The title compound was prepared similarly to the procedure for tert-butyl (R)-10-((6-fluoro-4-oxoquinazolin-3(4H)-yl)methyl)-7-azaspiro[4.5]decane-7-carboxylate (Intermediate 27) except using quinazolin-4(3H)-one instead of 6-fluoroquinazolin-4(3H)-one. LCMS (Method B): R_{T}=1.68 min, m/z=398 [M+H]⁺. ¹H NMR (500 MHz, DMSO-d₆): δ 8.37 (s, 1H), 8.15 (dd, J=8.0, 1.5 Hz, 1H), 7.83 (ddd, J=8.5, 7.1, 1.6 Hz, 1H), 7.71-7.66 (m, 1H), 7.55 (ddd, J=8.2, 7.1, 1.2 Hz, 1H), 4.18 (dd, J=13.3, 3.0 Hz, 1H), 3.83-3.73 (m, 2H), 3.55 (s, 1H), 2.83-2.65 (m, 1H), 1.94-1.84 (m, 2H), 1.74-1.62 (m, 2H), 1.62-1.52 (m, 2H), 1.50-1.40 (m, 2H), 2H), 1.39 (s, 9H), 1.36-1.17 (m, 4H)

### Intermediate 61: (2S,5R)-5-(2,5-Difluorophenyl)-2-methylmorpholine

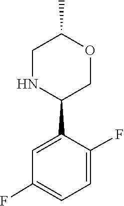

To a stirred solution of (S)-2-((tributylstannyl)methoxy)propan-1-amine (1.00 g, 2.65 mmol) [prepared according to Org. Lett., 2014, 16, 1236-1239 except using commercially available (S)-1-aminopropan-2-ol instead of 1-aminopropan-2-ol] in DCM (50 mL) was added 2,5-difluorobenzaldehyde (376 mg, 2.65 mmol), followed by finely powdered 4 A molecular sieves (5 g). After 16 h, the reaction was determined to be complete by NMR analysis to monitor consumption of the aldehyde (˜ 10.35 ppm in CDCl₃). The reaction mixture was filtered, the volatiles were removed in vacuo, resulting crude material of (S,E/Z)—N-(2,5-difluorobenzylidene)-2-((tributylstannyl)methoxy)propan-1-amine was dissolved in 1:1 DCM/hexafluoro-2-propanol (20 mL). This solution was added to a pre-prepared suspension of anhydrous copper (II) triflate (958 mg, 2.65 mmol) in hexafluoro-2-propanol (50 mL) in which 2,6-lutidine (284 mg, 2.65 mmol) was added and stirred for 6 h until the colour changed from light-blue to deep-green. The resulting mixture was stirred at rt. After 2 days, 25% NH_{3(aq)} (50 mL) and brine (50 mL) were added and the resulting biphasic mixture was stirred for 30 min. The resulting biphasic mixture was separated, the aqueous phase was extracted using DCM (2×50 mL), the combined organic phase was dried (Na₂SO₄), the solvents were removed in vacuo, and crude product was purified by flash chromatography to give the title compound (248 mg, 44%). LCMS (Method F): R_{T}=0.74 min, m/z=214 [M+H]⁺. ¹H NMR (400 MHz, DMSO-d₆): δ 1.06 (d, J=6.2 Hz, 3H), 2.83-2.89 (m, 1H), 2.93 (dd, J=11.9, 2.4 Hz, 1H), 3.16 (t, J=10.4 Hz, 1H), 3.46-3.58 (m, 1H), 3.73 (dd, J=10.7, 3.1 Hz, 1H), 3.93-4.05 (m, 1H), 7.15 (td, J=8.7, 8.2, 3.8 Hz, 1H), 7.22 (td, J=9.3, 4.6 Hz, 1H), 7.34 (ddd, J=9.3, 5.5, 3.3 Hz, 1H).

### Example 1: (R)-6-Phenyl-3-((1-(2-phenylpiperazine-1-carbonyl)piperidin-4-yl)methyl)pyrimidin-4(3H)-one

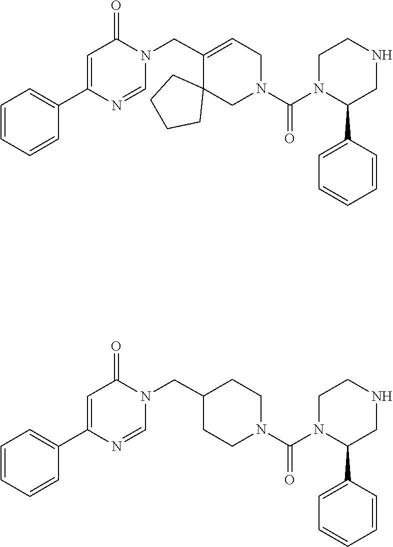 [SMILES: O=C(N1CC=C(Cn2cnc(-c3ccccc3)cc2=O)C2(CCCC2)C1)N1CCNC[C@H]1c1ccccc1 ; O=C(N1CCC(Cn2cnc(-c3ccccc3)cc2=O)CC1)N1CCNC[C@H]1c1ccccc1]

Step 1: tert-Butyl 4-((6-oxo-4-phenylpyrimidin-1(6H)-yl)methyl)piperidine-1-carboxylate: A stirring solution of 6-phenylpyrimidin-4(3H)-one (106 mg, 0.62 mmol), tert-butyl 4-(iodomethyl)piperidine-1-carboxylate (200 mg, 0.62 mmol) and cesium carbonate (401 mg, 1.23 mmol) in anhydrous 1,4-dioxane (4 mL) was heated at rt for 16 h then at 120° C. for 20 h. The mixture was partitioned between DCM (20 mL) and brine/water (1:1, 40 mL). The aqueous was separated and extracted with DCM (3×10 mL). The combined DCM fractions were dried (phase separator), filtered and reduced in vacuo. The residue was purified by flash chromatography (0-100% EtOAc in cyclohexane) to give the title compound (187 mg, 82%). LCMS (Method B): R_{T}=1.32 min, m/z 314 [M−butene+H]⁺.

Step 2: 6-Phenyl-3-(piperidin-4-ylmethyl)pyrimidin-4(3H)-one: Prepared according to General Procedure 1 using tert-butyl 4-((6-oxo-4-phenylpyrimidin-1(6H)-yl)methyl)piperidine-1-carboxylate (187 mg, 0.51 mmol) and TFA (2.3 mL), stirring for 20 min to give the title compound (134 mg, 98%). LCMS (Method B): R_{T}=0.57 min, m/z 270 [M+H]⁺.

Step 3: tert-Butyl (R)-4-(4-((6-oxo-4-phenylpyrimidin-1(6H)-yl)methyl)piperidine-1-carbonyl)-3-phenylpiperazine-1-carboxylate: Prepared according to General Procedure 2 using triphosgene (13.8 mg, 0.046 mmol), pyridine (37.4 μL, 0.46 mmol), tert-butyl (R)-3-phenylpiperazine-1-carboxylate (29.2 mg, 0.11 mmol) [commercially available], 6-phenyl-3-(piperidin-4-ylmethyl)pyrimidin-4(3H)-one (25.0 mg, 0.093 mmol) and DIPEA (81.1 μL, 0.46 mmol) to give the title compound (48.6 mg, 94%). LCMS (Method B): R_{T}=1.48 min, m/z 502 [M−butene+H]⁺.

Step 4: (R)-6-Phenyl-3-((1-(2-phenylpiperazine-1-carbonyl)piperidin-4-yl)methyl)pyrimidin-4(3H)-one: Prepared according to General Procedure 1 using tert-butyl (R)-4-(4-((6-oxo-4-phenylpyrimidin-1(6H)-yl)methyl)piperidine-1-carbonyl)-3-phenylpiperazine-1-carboxylate (48.6 mg, 0.087 mmol) and TFA (1.0 mL), stirring for 20 min to give the title compound (36.1 mg, 89%). LCMS (Method B): R_{T}=0.77 min, m/z 458 [M+H]⁺. ¹H NMR (500 MHz, DMSO-d₆): δ 8.53 (s, 1H), 8.09-8.02 (m, 2H), 7.53-7.46 (m, 3H), 7.32-7.26 (m, 4H), 7.20-7.14 (m, 1H), 6.97 (s, 1H), 4.48-4.43 (m, 1H), 3.87-3.79 (m, 2H), 3.79-3.72 (m, 2H), 3.09-2.98 (m, 3H), 2.96-2.90 (m, 1H), 2.81-2.71 (m, 3H), 2.70-2.62 (m, 1H), 2.04-1.93 (m, 1H), 1.62-1.48 (m, 2H), 1.22-1.09 (m, 2H). NH signal not observed.

### Examples 2 and 3: (R)-6-Phenyl-3-((7-(2-phenylpiperazine-1-carbonyl)-7-azaspiro[4.5]dec-9-en-10-yl)methyl)pyrimidin-4(3H)-one and (R,Z)-6-phenyl-3-((7-(2-phenylpiperazine-1-carbonyl)-7-azaspiro[4.5]decan-10-ylidene)methyl)pyrimidin-4(3H)-one

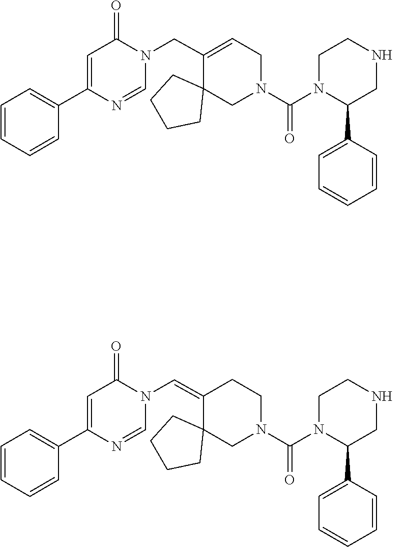 [SMILES: O=C(N1CC=C(Cn2cnc(-c3ccccc3)cc2=O)C2(CCCC2)C1)N1CCNC[C@H]1c1ccccc1 ; O=C(N1CC/C(=C/n2cnc(-c3ccccc3)cc2=O)C2(CCCC2)C1)N1CCNC[C@H]1c1ccccc1]

Step 1: tert-Butyl (R)-10-hydroxy-10-((6-oxo-4-phenylpyrimidin-1(6H)-yl)methyl)-7-azaspiro[4.5]decane-7-carboxylate: A stirring suspension of tert-butyl (R)-10-((4-chloro-6-oxopyrimidin-1(6H)-yl)methyl)-10-hydroxy-7-azaspiro[4.5]decane-7-carboxylate (500 mg, 1.26 mmol) [prepared according to WO2019/150119: Example 210, Step 1 therein], phenylboronic acid (306 mg, 2.51 mmol), sodium carbonate (400 mg, 3.77 mmol) and Pd(dppf)Cl₂·DCM (53.9 mg, 0.063 mmol) in 1,4-dioxane (9 mL) and water (3 mL) was degassed (under vacuum and backfilling with nitrogen, three times) in a sealed microwave vessel. The mixture was heated via microwave irradiation to 120° C. for 1 h. After cooling, the mixture was poured into water and stirred for 15 min. The solid was collected by filtration under vacuum, washing with water (100 mL), and dried under vacuum at 50° C. for 68 h to give crude title compound (583 mg, 100%) that was used in the next step without further purification. LCMS (Method B): R_{T}=1.44 min, m/z 384 [M−butene+H]⁺.

Step 2: tert-Butyl (Z)-10-((6-oxo-4-phenylpyrimidin-1(6H)-yl)methylene)-7-azaspiro[4.5]decane-7-carboxylate and tert-butyl 10-((6-oxo-4-phenylpyrimidin-1(6H)-yl)methyl)-7-azaspiro[4.5]dec-9-ene-7-carboxylate: To a stirring solution of tert-butyl (R)-10-hydroxy-10-((6-oxo-4-phenylpyrimidin-1(6H)-yl)methyl)-7-azaspiro[4.5]decane-7-carboxylate (583 mg, 1.33 mmol) in anhydrous DCM (5 mL) under a nitrogen atmosphere was added pyridine (0.32 mL, 3.98 mmol) and the solution was cooled to 0° C. Thionyl chloride (0.19 mL, 2.65 mmol) was added dropwise and the mixture stirred overnight while warming to rt. The mixture was partitioned between DCM (20 mL) and water (20 mL). The aqueous was separated and extracted with DCM (3×10 mL). The combined DCM fractions were dried (phase separator) and reduced in vacuo. The residue was purified by flash chromatography (0-50% EtOAc in cyclohexane) to give the mixture of title compounds (165 mg, 15%). LCMS (Method B): R_{T}=1.56 min, m/z 422 [M+H]⁺.

Step 3: (Z)-3-((7-Azaspiro[4.5]decan-10-ylidene)methyl)-6-phenylpyrimidin-4(3H)-one and 3-((7-azaspiro[4.5]dec-9-en-10-yl)methyl)-6-phenylpyrimidin-4(3H)-one (mixture): Prepared according to General Procedure 1 using the mixture of tert-butyl (Z)-10-((6-oxo-4-phenylpyrimidin-1(6H)-yl)methylene)-7-azaspiro[4.5]decane-7-carboxylate and tert-butyl 10-((6-oxo-4-phenylpyrimidin-1(6H)-yl)methyl)-7-azaspiro[4.5]dec-9-ene-7-carboxylate (50.0 mg, 0.059 mmol) and TFA (0.5 ml), stirring for 20 min to give the mixture of title compounds (35.4 mg, 93%). LCMS (Method B): R_{T}=0.70 min, 322 [M+H]⁺.

Step 4: tert-Butyl (R,Z)-4-(10-((6-oxo-4-phenylpyrimidin-1(6H)-yl)methylene)-7-azaspiro[4.5]decane-7-carbonyl)-3-phenylpiperazine-1-carboxylate and tert-butyl (R)-4-(10-((6-oxo-4-phenylpyrimidin-1(6H)-yl)methyl)-7-azaspiro[4.5]dec-9-ene-7-carbonyl)-3-phenylpiperazine-1-carboxylate: Prepared according to General Procedure 2 using triphosgene (8.2 mg, 0.028 mmol), pyridine (22.2 μL, 0.28 mmol), tert-butyl (R)-3-phenylpiperazine-1-carboxylate (17.3 mg, 0.066 mmol), the mixture of (Z)-3-((7-azaspiro[4.5]decan-10-ylidene)methyl)-6-phenylpyrimidin-4(3H)-one and 3-((7-azaspiro[4.5]dec-9-en-10-yl)methyl)-6-phenylpyrimidin-4(3H)-one (17.7 mg, 0.028 mmol) and DIPEA (48.1 μL, 0.28 mmol) to give the mixture of title compounds (32.8 mg, 98%). LCMS (Method B): R_{T}=1.63 min, m/z 610 [M+H]⁺.

Step 5: (R)-6-Phenyl-3-((7-(2-phenylpiperazine-1-carbonyl)-7-azaspiro[4.5]dec-9-en-10-yl)methyl)pyrimidin-4(3H)-one and (R,Z)-6-phenyl-3-((7-(2-phenylpiperazine-1-carbonyl)-7-azaspiro[4.5]decan-10-ylidene)methyl)pyrimidin-4(3H)-one: Prepared according to General Procedure 1 using the mixture of tert-butyl (R,Z)-4-(10-((6-oxo-4-phenylpyrimidin-1(6H)-yl)methylene)-7-azaspiro[4.5]decane-7-carbonyl)-3-phenylpiperazine-1-carboxylate and tert-butyl (R)-4-(10-((6-oxo-4-phenylpyrimidin-1(6H)-yl)methyl)-7-azaspiro[4.5]dec-9-ene-7-carbonyl)-3-phenylpiperazine-1-carboxylate (32.8 mg, 0.0538 mmol) and TFA (0.3 mL), stirring for 20 min to give the mixture of title compounds. The mixture was purified by preparative HPLC using the following gradient:

| Gradient Time (min) | Flow (mL/min) | % A | % B |
|---|---|---|---|
| 0.00 | 85.0 | 83 | 17 |
| 3.00 | 85.0 | 67 | 33 |
| 5.00 | 85.0 | 67 | 33 |
| 7.00 | 85.0 | 62 | 38 |
| 14.00 | 85.0 | 62 | 38 |
| 15.00 | 85.0 | 0 | 100 |
| 16.00 | 85.0 | 0 | 100 |

The separated residues were each dissolved in MeCN (2 mL) and water (10 mL) then freeze-dried to afford (R)-6-phenyl-3-((7-(2-phenylpiperazine-1-carbonyl)-7-azaspiro[4.5]dec-9-en-10-yl)methyl)pyrimidin-4(3H)-one (first eluting compound: 6.2 mg, 22% yield). LCMS (Method B): R_{T}=0.96 min, m/z 510 [M+H]⁺. ¹H NMR (500 MHz, DMSO-d₆): δ 8.49 (s, 1H), 8.12-8.06 (m, 2H), 7.53-7.47 (m, 3H), 7.31-7.23 (m, 4H), 7.22-7.17 (m, 1H), 7.01 (s, 1H), 5.02-4.98 (m, 1H), 4.57-4.51 (m, 2H), 4.30 (dd, 1H), 4.00-3.83 (m, 2H), 3.27-3.24 (m, 2H), 3.18-2.82 (m, 7H), 1.77-1.69 (m, 1H), 1.69-1.52 (m, 5H), 1.38-1.25 (m, 2H) and (R,Z)-6-phenyl-3-((7-(2-phenylpiperazine-1-carbonyl)-7-azaspiro[4.5]decan-10-ylidene)methyl)pyrimidin-4(3H)-one (second eluting compound: 8.6 mg, 30%). LCMS (Method B): R_{T}=0.93 min, m/z 510 [M+H]⁺. ¹H NMR (500 MHz, DMSO-d₆): δ 8.32 (s, 1H), 8.12-8.05 (m, 2H), 7.54-7.46 (m, 3H), 7.37-7.27 (m, 4H), 7.25-7.15 (m, 1H), 7.01 (s, 1H), 6.42 (s, 1H), 4.47-4.40 (m, 1H), 3.46-3.32 (m, 2H), 3.23-2.96 (m, 6H), 2.92-2.81 (m, 2H), 2.23-2.12 (m, 2H), 1.74-1.56 (m, 6H), 1.56-1.41 (m, 2H), 1.29-1.20 (m, 1H).

### Example 4: 6-Phenyl-3-((7-((R)-4,4,4-trifluoro-2-methylbutanoyl)-7-azaspiro[4.5]decan-10-yl)methyl)pyrimidin-4(3H)-one

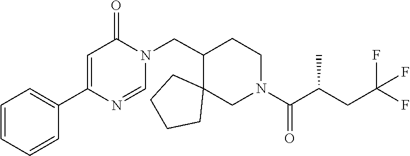 [SMILES: C[C@H](CC(F)(F)F)C(=O)N1CCC(Cn2cnc(-c3ccccc3)cc2=O)C2(CCCC2)C1]

Step 1: tert-Butyl 10-oxo-7-azaspiro[4.5]decane-7-carboxylate: Potassium tert-butoxide (24.8 g, 221 mmol) was added in portions to a stirring solution of tert-butyl 4-oxopiperidine-1-carboxylate (20.0 g, 100 mmol) in toluene (200 ml) under an N₂ atmosphere. The mixture was stirred for 1 h. 1,4-Dibromobutane (12.0 mL, 100 mmol) was added dropwise over 15 min then the mixture heated to reflux for 2 h. The mixture was cooled then partitioned between 1:1 saturated ammonium chloride (aq) solution/water (200 mL) and EtOAc (75 mL). The aqueous was separated and extracted with EtOAc (2×75 mL). The combined EtOAc fractions were dried (Na₂SO₄), filtered (phase separator) and reduced in vacuo. The residue was purified by flash chromatography (0-10% EtOAc in cyclohexane) to give the title compound (9.4 g, 37%). ¹H NMR (500 MHz, CDCl₃): 3.71 (t, 2H), 3.45 (s, 2H), 2.48 (t, 2H), 1.97-1.88 (m, 2H), 1.72-1.62 (m, 4H), 1.49 (s, 9H), 1.51-1.44 (m, 2H).

Step 2: tert-Butyl 10-methylene-7-azaspiro[4.5]decane-7-carboxylate: To a stirring solution of (methyl)triphenylphosphoniumbromide (6.2 g, 17.3 mmol) in anhydrous THF (55 mL) under an N₂ atmosphere at −78° C. was added 2.5 M n-butyl lithium in hexanes (5.5 mL, 13.7 mmol) dropwise. The cooling was removed and stirring continued at rt for 45 min. A solution of tert-butyl 10-oxo-7-azaspiro[4.5]decane-7-carboxylate (3.7 g, 14.4 mmol) in anhydrous THF (20 mL) was added dropwise and the mixture stirred for 3 h. The mixture was then heated at reflux for 40 h. After cooling, the solvent was removed in vacuo. The residue was triturated in diethyl ether (150 mL). The solid was removed by filtration (silica plug) and the filtrate was reduced in vacuo. The residue was triturated in diethyl ether (50 mL). The solid was removed by filtration (silica plug) and the filtrate was reduced in vacuo. The remaining residue was purified by flash chromatography (0-10% EtOAc in cyclohexane) to give the title compound (2.8 g, 78%). ¹H NMR (500 MHz, CDCl₃): δ 4.70 (d, 2H), 2.52-3.35 (m, 2H), 3.18 (s, 2H), 2.24 (t, 2H), 1.73-1.58 (m, 6H), 1.55-1.49 (m, 2H), 1.47 (s, 9H).

Step 3: tert-Butyl 10-(hydroxymethyl)-7-azaspiro[4.5]decane-7-carboxylate: To a stirring solution of tert-butyl 10-methylene-7-azaspiro[4.5]decane-7-carboxylate (5.1 g, 20.3 mmol) in anhydrous THF (145 ml) under an N₂ atmosphere at 0° C. was added 0.5 M solution of 9-borabicyclo[3.3.1]nonane in THF (97.6 mL, 48.8 mmol) dropwise. The mixture was stirred while warming to rt for 16 h. A 5 M solution of sodium acetate (aq) (14.1 mL, 70.5 mmol) was added followed by hydrogen peroxide (30% w/w in water) (14.1 mL, 138.1 mmol). Stirring was continued for 68 h. The mixture was partitioned between EtOAc (750 mL) and saturated sodium hydrogen carbonate (aq) solution (750 mL). The aqueous was separated and extracted with EtOAc (2×250 mL). The combined EtOAc fractions were dried (Na₂SO₄), filtered and reduced in vacuo. The residue was purified by flash chromatography (0-100% EtOAc in cyclohexane) to give the title compound (4.7 g, 86%). ¹H NMR (500 MHz, CDCl₃): δ 4.04-3.85 (m, 1H), 3.81 (dd, 1H), 3.68-3.49 (m, 1H), 3.45 (t, 1H), 2.92 (t, 1H), 2.62 (d, 1H), 1.82 (dq, 1H), 1.72-1.56 (m, 5H), 1.54-1.46 (m, 2H), 1.45 (s, 9H), 1.43-1.18 (4H).

Step 4: tert-Butyl 10-(bromomethyl)-7-azaspiro[4.5]decane-7-carboxylate: To a stirring solution of tert-butyl 10-(hydroxymethyl)-7-azaspiro[4.5]decane-7-carboxylate (4.7 g, 17.5 mmol) and triphenylphosphine (6.0 g, 22.8 mmol) in anhydrous DCM (150 mL) at 0° C. was added carbon tetrabromide (7.6 g, 22.8 mmol). The mixture was warmed to rt and stirred for 40 h. The solvent was removed in vacuo. The residue was purified by flash chromatography (0-20% EtOAc in cyclohexane) to give the title compound (4.6 g, 79%). ¹H NMR (500 MHz, CDCl₃): δ 4.04-3.78 (m, 1H), 3.68-3.48 (m, 2H), 3.15 (t, 1H), 2.92 (t, 1H), 2.61 (d, 1H), 2.05-1.95 (m, 1H), 1.78 (tt, 1H), 1.74-1.67 (m, 1H), 1.67-1.54 (m, 3H), 1.53-1.42 (m, 2H), 1.45 (s, 9H), 1.39-1.28 (m, 2H), 1.25-1.15 (m, 1H).

Step 5: tert-Butyl 10-((6-oxo-4-phenylpyrimidin-1(6H)-yl)methyl)-7-azaspiro[4.5]decane-7-carboxylate: To a stirring solution of tert-butyl 10-(bromomethyl)-7-azaspiro[4.5]decane-7-carboxylate (110 mg, 0.33 mmol) in anhydrous DMF (2 mL) was added 6-phenylpyrimidin-4(3H)-one (57.0 mg, 0.33 mmol) followed by cesium carbonate (21 mg, 0.66 mmol). The mixture was heated at 80° C. for 16 h. The mixture was partitioned between EtOAc (5 mL) and 1:1 brine/water (20 mL). The aqueous was separated and extracted with EtOAc (3×5 mL). The combined EtOAc fractions were washed with 1:1 brine/water (4×5 mL), dried (Na₂SO₄), filtered and reduced in vacuo. The residue was purified by flash chromatography (0-50% EtOAc in cyclohexane) to give the title compound (30.8 mg, 22%). LCMS (Method B): R_{T}=1.57 min, m/z 368 [M−butene+H]⁺.

Step 6: 3-((7-Azaspiro[4.5]decan-10-yl)methyl)-6-phenylpyrimidin-4(3H)-one: Prepared according to General Procedure 1 using tert-butyl 10-((6-oxo-4-phenylpyrimidin-1(6H)-yl)methyl)-7-azaspiro[4.5]decane-7-carboxylate (15.6 mg, 0.037 mmol) and TFA (0.25 mL), stirring for 20 min to give the title compound (10.1 mg, 85%). LCMS (Method B): R_{T}=0.73 min, m/z 324 [M+H]⁺.

Step 7: 6-Phenyl-3-((7-((R)-4,4,4-trifluoro-2-methylbutanoyl)-7-azaspiro[4.5]decan-10-yl)methyl)pyrimidin-4(3H)-one: Prepared according to General Procedure 3 using 3-((7-azaspiro[4.5]decan-10-yl)methyl)-6-phenylpyrimidin-4(3H)-one (10.0 mg, 0.031 mmol), (R)-4,4,4-trifluoro-2-methylbutanoic acid (5.3 mg, 0.034 mmol) [prepared according to WO2019/150119: Acid 3], HATU (14.1 mg, 0.037 mmol) and DIPEA (16.2 μL, 0.093 mmol) to give the title compound (8.3 mg, 56%). LCMS (Method B): R_{T}=1.42 min, m/z 462 [M+H]⁺. ¹H NMR (500 MHz, DMSO-d₆): δ 8.62-8.51 (m, 1H), 8.13-8.00 (m, 2H), 7.57-7.42 (m, 3H), 6.97 (s, 1H), 4.32-3.39 (m, 4H), 3.19-2.94 (m, 2H), 2.88-2.65 (m, 1H), 2.61-2.54 (m, 1H), 2.34-2.18 (m, 1H), 2.00-1.81 (m, 2H), 1.78-1.48 (m, 4H), 1.46-1.14 (m, 5H), 1.14-1.01 (m, 3H).

### Example 5: 4-Phenyl-1-((7-((R)-4,4,4-trifluoro-2-methylbutanoyl)-7-azaspiro[4.5]decan-10-yl)methyl)pyridin-2(1H)-one

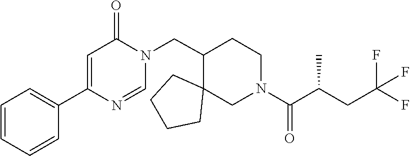 [SMILES: C[C@H](CC(F)(F)F)C(=O)N1CCC(Cn2cnc(-c3ccccc3)cc2=O)C2(CCCC2)C1]

Step 1: tert-Butyl 10-((2-oxo-4-phenylpyridin-1(2H)-yl)methyl)-7-azaspiro[4.5]decane-7-carboxylate: To a stirring solution of tert-butyl 10-(bromomethyl)-7-azaspiro[4.5]decane-7-carboxylate (100 mg, 0.30 mmol) in anhydrous DMF (2 mL) was added 4-phenylpyridin-2(1H)-one (51.5 mg, 0.30 mmol) followed by cesium carbonate (196 mg, 0.60 mmol). The mixture was heated at rt for 72 h. Separately, to a stirring solution of tert-butyl 10-(bromomethyl)-7-azaspiro[4.5]decane-7-carboxylate (200 mg, 0.60 mmol) in anhydrous DMF (4 mL) was added 4-phenylpyridin-2(1H)-one (103 mg, 0.60 mmol) followed by cesium carbonate (392 mg, 1.20 mmol). The mixture was heated at 80° C. for 16 h. After cooling, the combined mixtures were partitioned between EtOAc (15 mL) and 1:1 brine/water (60 mL). The aqueous was separated and extracted with EtOAc (3×15 mL). The combined EtOAc fractions were washed with 1:1 brine/water (4×15 mL), dried (Na₂SO₄), filtered and reduced in vacuo. The residue was purified by flash chromatography (0-100% EtOAc in cyclohexane) to give the title compound (33.8 mg, 13%). LCMS (Method B): R_{T}=1.56 min, m/z 367 [M−butene+H]⁺.

Step 2: 1-((7-Azaspiro[4.5]decan-10-yl)methyl)-4-phenylpyridin-2(1H)-one: Prepared according to General Procedure 1 using tert-butyl 10-((2-oxo-4-phenylpyridin-1(2H)-yl)methyl)-7-azaspiro[4.5]decane-7-carboxylate (33.8 mg, 0.080 mmol) and TFA (0.3 mL), stirring for 20 min to give the title compound (13.5 mg, 52%). LCMS (Method B): R_{T}=0.79 min, m/z 323 [M+H]⁺.

Step 3: 4-Phenyl-1-((7-((R)-4,4,4-trifluoro-2-methylbutanoyl)-7-azaspiro[4.5]decan-10-yl)methyl)pyridin-2(1H)-one: Prepared according to General Procedure 3 using 1-((7-azaspiro[4.5]decan-10-yl)methyl)-4-phenylpyridin-2(1H)-one (10.0 mg, 0.0310 mmol), (R)-4,4,4-trifluoro-2-methylbutanoic acid (5.3 mg, 0.034 mmol) [prepared according to WO2019/150119: Acid 3], HATU (14.2 mg, 0.037 mmol) and DIPEA (16.3 μL, 0.093 mmol) to give the title compound (9 mg, 60%). LCMS (Method B): R_{T}=1.41 min, m/z 461 [M+H]⁺. ¹H NMR (500 MHz, DMSO-d₆): δ 7.78-7.68 (m, 3H), 7.52-7.43 (m, 3H), 6.68-6.63 (m, 1H), 6.60-6.55 (m, 1H), 4.30-3.39 (m, 4H), 3.19-2.96 (m, 2H), 2.88-2.56 (m, 2H), 2.33-2.19 (m, 1H), 2.02-1.72 (m, 2H), 1.72-1.46 (m, 4H), 1.46-1.15 (m, 5H), 1.14-0.98 (m, 3H).

### Example 6: (R)-4-Phenyl-1-((1-(2-phenylpiperazine-1-carbonyl)piperidin-4-yl)methyl)pyridin-2(1H)-one

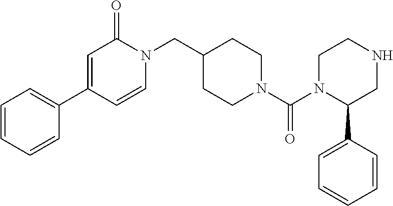 [SMILES: O=C(N1CCC(Cn2ccc(-c3ccccc3)cc2=O)CC1)N1CCNC[C@H]1c1ccccc1]

Step 1: tert-Butyl 4-((2-oxo-4-phenylpyridin-1(2H)-yl)methyl)piperidine-1-carboxylate: A stirring solution of 4-phenylpyridin-2(1H)-one (105 mg, 0.62 mmol), tert-butyl 4-(iodomethyl)piperidine-1-carboxylate (200 mg, 0.62 mmol) and cesium carbonate (401 mg, 1.23 mmol) in anhydrous 1,4-dioxane (4 mL) was heated at rt for 16 h then 120° C. for 20 h. The mixture was partitioned between DCM (20 mL) and 1:1 brine/water (40 mL). The aqueous was separated and extracted with DCM (3×10 mL). The combined DCM fractions were dried (phase separator), filtered and reduced in vacuo. The residue was purified by flash chromatography (0-100% EtOAc in cyclohexane) to give the title compound (132 mg, 58%). LCMS (Method B): R_{T}=1.31 min, m/z 313 [M−butene+H]⁺.

Step 2: 4-Phenyl-1-(piperidin-4-ylmethyl)pyridin-2(1H)-one: Prepared according to General Procedure 1 using tert-butyl 4-((2-oxo-4-phenylpyridin-1(2H)-yl)methyl)piperidine-1-carboxylate (132 mg, 0.36 mmol) and TFA (1.0 mL, 13.0 mmol), stirring for 20 min to give the title compound (86.7 mg, 90%). LCMS (Method B): R_{T}=0.55 min, m/z 269 [M+H]⁺.

Step 3: tert-Butyl (R)-4-(4-((2-oxo-4-phenylpyridin-1(2H)-yl)methyl)piperidine-1-carbonyl)-3-phenylpiperazine-1-carboxylate: Prepared according to General Procedure 2 using triphosgene (13.8 mg, 0.047 mmol), pyridine (37.5 μL, 0.47 mmol), tert-butyl (R)-3-phenylpiperazine-1-carboxylate (29.3 mg, 0.11 mmol), 4-phenyl-1-(piperidin-4-ylmethyl)pyridin-2(1H)-one (25.0 mg, 0.093 mmol) and DIPEA (81.4 μL, 0.47 mmol) to give the title compound (49.7 mg, 95%). LCMS (Method B): R_{T}=1.47 min, m/z 557 [M+H]⁺.

Step 4: (R)-4-Phenyl-1-((1-(2-phenylpiperazine-1-carbonyl)piperidin-4-yl)methyl)pyridin-2(1H)-one: Prepared according to General Procedure 1 using tert-butyl (R)-4-(4-((2-oxo-4-phenylpyridin-1(2H)-yl)methyl)piperidine-1-carbonyl)-3-phenylpiperazine-1-carboxylate (49.7 mg, 0.089 mmol) and TFA (1.0 mL, 0.18 mmol), stirring for 20 min to give the title compound (17.7 mg, 41%). LCMS (Method B): R_{T}=0.83 min, m/z 457 [M+H]⁺. ¹H NMR (500 MHz, DMSO-d₆): δ 7.77-7.68 (m, 3H), 7.52-7.43 (m, 3H), 7.33-7.25 (m, 4H), 7.21-7.14 (m, 1H), 6.68-6.64 (m, 1H), 6.59-6.54 (m, 1H), 4.45 (t, 1H), 3.86-3.78 (m, 2H), 3.78-3.71 (m, 2H), 3.28-3.25 (m, 1H), 3.09-2.97 (m, 3H), 2.95-2.88 (m, 1H), 2.81-2.70 (m, 3H), 2.69-2.64 (m, 1H), 2.04-1.93 (m, 1H), 1.60-1.46 (m, 2H), 1.22-1.09 (m, 2H).

### Example 7: 6-Phenyl-3-((7-((S)-4,4,4-trifluoro-2-(methoxymethyl)butanoyl)-7-azaspiro[4.5]decan-10-yl)methyl)pyrimidin-4(3H)-one

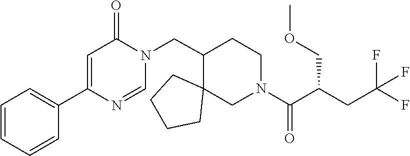 [SMILES: COC[C@H](CC(F)(F)F)C(=O)N1CCC(Cn2cnc(-c3ccccc3)cc2=O)C2(CCCC2)C1]

Step 1: (R)-4-Benzyl-3-(4,4,4-trifluorobutanoyl)oxazolidin-2-one: To (R)-4-benzyloxazolidin-2-one (8.06 g, 45.5 mmol) in THF (100 mL) at −78° C. was added 2.5M solution of BuLi in THF (45.5 mL, 114 mmol). After 15 min, 3-trifluoromethylpropanoyl chloride (7.30 g, 45.5 mmol) was added. After 24 h, the reaction mixture was partitioned using saturated NH₄Cl (aq) solution. The organic phase was separated, dried (Na₂SO₄) and evaporated to dryness. The crude material was purified by flash chromatography to give the title compound (2.80 g, 20%). ¹H NMR (400 MHz, CDCl₃): δ 7.38-7.15 (m, 5H), 4.70 (m, 1H), 4.22 (m, 2H), 3.30-3.15 (m, 3H), 2.74 (m, 1H), 2.56 (m, 2H).

Step 2: (R)-4-Benzyl-3-((S)-4,4,4-trifluoro-2-(methoxymethyl)butanoyl)oxazolidin-2-one: To (R)-4-benzyl-3-(4,4,4-trifluorobutanoyl)oxazolidin-2-one (2.60 g, 8.6 mmol) in THF (70 mL) at −70° C. was added 1.7M NaHMDS in THF solution (7.5 mL) followed by methoxymethylbromide (1.62 g, 13 mmol). After 24 h, the reaction mixture was partitioned with saturated NH₄Cl (aq) solution. The organic phase was separated, dried (Na₂SO₄) and evaporated to dryness. The crude material was purified by flash chromatography to give the title compound (0.75 g, 25%). LCMS (Method F): R_{T}=1.50 min, m/z 346 [M+H]⁺.

Step 3: (S)-4,4,4-Trifluoro-2-(methoxymethyl)butanoic acid: To (R)-4-benzyl-3-((S)-4,4,4-trifluoro-2-(methoxymethyl)butanoyl)oxazolidin-2-one (0.60 g, 1.7 mmol) in 1:1 THF/water (20 mL) was added 30% hydrogen peroxide (aq) solution (0.79 mL, 6.8 mmol) and lithium hydroxide hydrate (0.15 g, 3.5 mmol). After 24 h, the volatiles were evaporated and the remaining aqueous mixture was extracted using ethyl acetate. The aqueous phase was acidified using sodium hydrogensulfate (aq) solution and extracted using DCM (×2). The organic phase was dried (Na₂SO₄) and evaporated to the title compound (0.14 g, 43%). GCMS (Method A): m/z 186 M+. ¹H NMR (400 MHz, CDCl₃): δ 3.69-3.57 (m, 2H), 3.35 (s, 3H), 2.95 (m, 1H), 2.70-2.59 (m, 1H), 2.45-2.35 (m, 1H).

Step 4: 6-Phenyl-3-((7-((S)-4,4,4-trifluoro-2-(methoxymethyl)butanoyl)-7-azaspiro[4.5]decan-10-yl)methyl)pyrimidin-4(3H)-one: Prepared according to General Procedure 3 using 3-((7-azaspiro[4.5]decan-10-yl)methyl)-6-phenylpyrimidin-4(3H)-one (10.0 mg, 0.031 mmol), (S)-4,4,4-trifluoro-2-(methoxymethyl)butanoic acid (6.3 mg, 0.034 mmol), HATU (14.1 mg, 0.037 mmol) and DIPEA (16.2 μL, 0.093 mmol) to give the title compound (9.5 mg, 60%). LCMS (Method B): R_{T}=1.40 min, m/z 492 [M+H]⁺. ¹H NMR (500 MHz, DMSO-d₆): δ 8.62-8.52 (m, 1H), 8.11-8.02 (m, 2H), 7.55-7.45 (m, 3H), 6.97 (s, 1H), 4.26 (d, 0.16H), 4.18-4.08 (m, 1H), 4.00 (d, 0.39H), 3.96-3.87 (m, 1H), 3.86-3.64 (m, 1H), 3.59 (d, 0.16), 3.50-3.42 (m, 0.34H), 3.42-3.33 (m, 2H), 3.28-3.19 (m, 3H), 3.12-2.82 (m, 1H), 2.82-2.64 (m, 1H), 2.62-2.54 (m, 1H), 2.44-2.29 (m, 1H), 2.00-1.90 (m, 1H), 1.89-1.73 (m, 1H), 1.72-1.02 (m, 10H).

### Example 8: 6-Phenyl-3-((7-((S)-2-phenylpyrrolidine-1-carbonyl)-7-azaspiro[4.5]decan-10-yl)methyl)pyrimidin-4(3H)-one

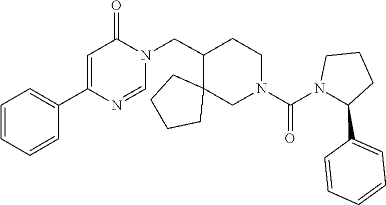 [SMILES: O=C(N1CCC(Cn2cnc(-c3ccccc3)cc2=O)C2(CCCC2)C1)N1CCC[C@H]1c1ccccc1]

Prepared according to General Procedure 2 using triphosgene (4.6 mg, 0.016 mmol), pyridine (12.5 μL, 0.15 mmol), (S)-2-phenylpyrrolidine (5.5 μL, 0.037 mmol), 3-((7-azaspiro[4.5]decan-10-yl)methyl)-6-phenylpyrimidin-4(3H)-one (10.0 mg, 0.031 mmol) and DIPEA (27.0 μL, 0.15 mmol to give the title compound (11.5 mg, 67%). LCMS (Method B): R_{T}=1.53 min, m/z 497 [M+H]⁺. ¹H NMR (500 MHz, DMSO-d₆): δ 8.58 (d, 1H), 8.10-8.00 (m, 2H), 7.54-7.44 (m, 3H), 7.30-7.16 (m, 5H), 6.95 (s, 1H), 4.90 (t, 1H), 4.15-4.02 (m, 1H), 3.84 (t, 0.5H), 3.75 (d, 0.5H), 3.68-3.55 (m, 1.5H), 3.51-3.43 (m, 1H), 3.40 (t, 0.5H), 3.34 (t, 1H), 3.27-3.25 (m, 0.5H), 2.98-2.89 (t, 0.5H), 2.58 (t, 1H), 2.31-2.22 (m, 1H), 1.94-1.80 (m, 2.5H), 1.80-1.72 (m, 1H), 1.72-1.46 (m, 6H), 1.46-1.35 (m, 1H), 1.33-1.15 (m, 3.5H).

### Example 9: 3-((1-((2S,4R)-4-Amino-2-phenylpiperidine-1-carbonyl)piperidin-4-yl)methyl)-6-phenylpyrimidin-4(3H)-one

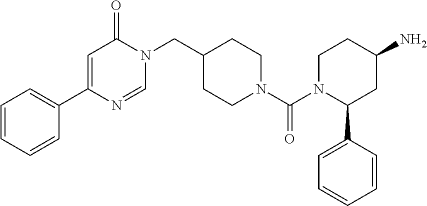 [SMILES: N[C@@H]1CCN(C(=O)N2CCC(Cn3cnc(-c4ccccc4)cc3=O)CC2)[C@H](c2ccccc2)C1]

Step 1: tert-Butyl ((2S,4R)-1-(4-((6-oxo-4-phenylpyrimidin-1(6H)-yl)methyl)piperidine-1-carbonyl)-2-phenylpiperidin-4-yl)carbamate: Prepared according to General Procedure 3 using triphosgene (5.5 mg, 0.019 mmol), pyridine (15.0 μL, 0.19 mmol), tert-butyl ((2S,4R)-2-phenylpiperidin-4-yl)carbamate (12.3 mg, 0.045 mmol), 6-phenyl-3-(piperidin-4-ylmethyl)pyrimidin-4(3H)-one (10.0 mg, 0.037 mmol) and DIPEA (32.4 μL, 0.19 mmol) to give the title compound (14.7 mg, 69%). LCMS (Method B): R_{T}=1.41 min, m/z 572 [M−butene+H]⁺.

Step 2: 3-((1-((2S,4R)-4-Amino-2-phenylpiperidine-1-carbonyl)piperidin-4-yl)methyl)-6-phenylpyrimidin-4(3H)-one: Prepared according to General Procedure 1 using tert-butyl ((2S,4R)-1-(4-((6-oxo-4-phenylpyrimidin-1(6H)-yl)methyl)piperidine-1-carbonyl)-2-phenylpiperidin-4-yl)carbamate (14.7 mg, 0.026 mmol) and TFA (0.3 mL), stirring for 20 min to give the title compound (10.9 mg, 87%). LCMS (Method B): R_{T}=0.78 min, m/z 472 [M+H]⁺. ¹H NMR (500 MHz, DMSO-d₆): δ 8.53 (s, 1H), 8.10-8.03 (m, 2H), 7.53-7.46 (m, 3H), 7.22 (t, 2H), 7.17 (d, 2H), 7.08 (t, 1H), 6.97 (s, 1H), 4.05-3.90 (m, 3H), 3.87-3.76 (m, 2H), 3.26-3.20 (m, 2H), 2.89-2.59 (m, 5H), 2.03-1.91 (m, 1H), 1.81 (d, 2H), 1.59 (d, 1H), 1.51 (d, 1H), 1.37 (q, 1H), 1.23 (q, 1H), 1.08 (dq, 1H), 0.99 (m, 1H).

### Example 10: N-Benzyl-10-((6-oxo-4-phenylpyrimidin-1(6H)-yl)methyl)-7-azaspiro[4.5]decane-7-carboxamide

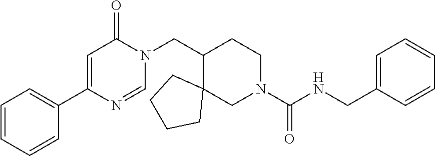 [SMILES: O=C(NCc1ccccc1)N1CCC(Cn2cnc(-c3ccccc3)cc2=O)C2(CCCC2)C1]

Prepared according to General Procedure 2 using triphosgene (4.6 mg, 0.016 mmol), pyridine (12.5 μL, 0.15 mmol), benzylamine (4.1 μL, 0.037 mmol), 3-((7-azaspiro[4.5]decan-10-yl)methyl)-6-phenylpyrimidin-4(3H)-one (10.0 mg, 0.031 mmol) and DIPEA (27.0 μL, 0.15 mmol) to give the title compound (9.3 mg, 65%). LCMS (Method B): R_{T}=1.34 min, m/z 457 [M+H]⁺. ¹H NMR (500 MHz, DMSO-d₆): δ 8.56 (s, 1H), 8.11-8.02 (m, 2H), 7.52-7.45 (m, 3H), 7.29 (t, 2H), 7.23 (d, 2H), 7.19 (t, 1H), 6.98 (t, 1H), 6.96 (s, 1H), 4.28-4.18 (m, 2H), 4.13 (dd, 1H), 3.90-3.82 (m, 1H), 3.72 (t, 1H), 3.61 (d, 1H), 2.78-2.69 (m, 1H), 2.59 (d, 1H), 1.90-1.79 (m, 2H), 1.74-1.65 (m, 1H), 1.65-1.57 (m, 2H), 1.57-1.48 (m, 1H), 1.48-1.39 (m, 2H), 1.36-1.27 (m, 2H), 1.26-1.17 (m, 1H).

### Example 11: 3-((7-((R)-2-(3,5-Difluorophenyl)piperazine-1-carbonyl)-7-azaspiro[4.5]decan-10-yl)methyl)-6-phenylpyrimidin-4(3H)-one

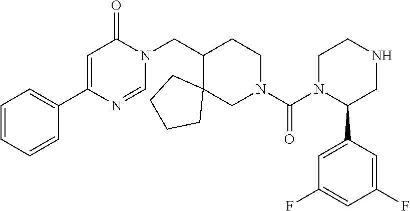 [SMILES: O=C(N1CCC(Cn2cnc(-c3ccccc3)cc2=O)C2(CCCC2)C1)N1CCNC[C@H]1c1cc(F)cc(F)c1]

Step 1: tert-butyl (3R)-3-(3,5-difluorophenyl)-4-(10-((6-oxo-4-phenylpyrimidin-1(6H)-yl)methyl)-7-azaspiro[4.5]decane-7-carbonyl)piperazine-1-carboxylate: Prepared according to General Procedure 2 using triphosgene (4.6 mg, 0.0156 mmol), pyridine (12.5 μL, 0.15 mmol), tert-butyl (R)-3-(3,5-difluorophenyl)piperazine-1-carboxylate (11.1 mg, 0.037 mmol) [Intermediate 2], 3-((7-azaspiro[4.5]decan-10-yl)methyl)-6-phenylpyrimidin-4(3H)-one (10.0 mg, 0.031 mmol) and DIPEA (27.0 μL, 0.15 mmol) to give the title compound (13.9 mg, 69%). LCMS (Method B): R_{T}=1.68 min, m/z 648 [M+H]⁺.

Step 2: 3-((7-((R)-2-(3,5-Difluorophenyl)piperazine-1-carbonyl)-7-azaspiro[4.5]decan-10-yl)methyl)-6-phenylpyrimidin-4(3H)-one: Prepared according to General Procedure 1 using tert-butyl (3R)-3-(3,5-difluorophenyl)-4-(10-((6-oxo-4-phenylpyrimidin-1(6H)-yl)methyl)-7-azaspiro[4.5]decane-7-carbonyl)piperazine-1-carboxylate (13.9 mg, 0.022 mmol) and TFA (0.3 mL), stirring for 20 min to give the title compound (11.6 mg, 98%). LCMS (Method B): R_{T}=1.04 min, m/z 548 [M+H]⁺. ¹H NMR (500 MHz, DMSO-d₆): δ 8.59-8.52 (m, 1H), 8.09-8.02 (m, 2H), 7.53-7.46 (m, 3H), 7.09-7.03 (m, 1H), 7.00 (t, 2H), 6.96 (s, 1H), 4.41-4.27 (m, 1H), 4.15-4.03 (m, 1H), 3.82-3.68 (m, 2H), 3.43-3.32 (m, 1H), 3.28-3.23 (m, 1H), 3.13-3.01 (m, 1H), 3.01-2.90 (m, 3H), 2.88-2.75 (m, 2H), 2.74-2.59 (m, 1H), 1.92-1.74 (m, 2H), 1.70-1.44 (m, 4H), 1.44-1.20 (m, 5H).

### Example 12: 3-((7-((3R,4R)-1-Imino-1-oxido-4-phenyltetrahydro-1H-1λ⁶-thiophene-3-carbonyl)-7-azaspiro[4.5]decan-10-yl)methyl)-6-phenylpyrimidin-4(3H)-one and 3-((7-((3S,4S)-1-imino-1-oxido-4-phenyltetrahydro-1H-1λ⁶-thiophene-3-carbonyl)-7-azaspiro[4.5]decan-10-yl)methyl)-6-phenylpyrimidin-4(3H)-one

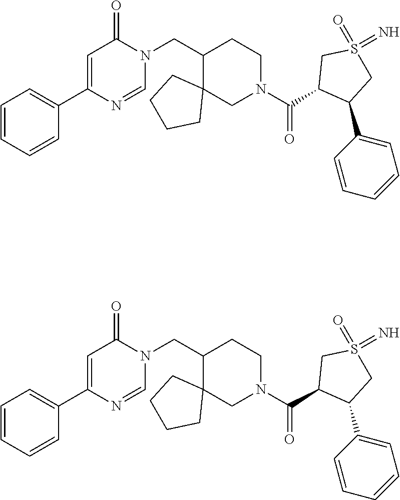 [SMILES: N=S1(=O)C[C@@H](C(=O)N2CCC(Cn3cnc(-c4ccccc4)cc3=O)C3(CCCC3)C2)[C@H](c2ccccc2)C1 ; N=S1(=O)C[C@H](C(=O)N2CCC(Cn3cnc(-c4ccccc4)cc3=O)C3(CCCC3)C2)[C@@H](c2ccccc2)C1]

Step 1: rac-6-Phenyl-3-((7-((3R,4R)-4-phenyltetrahydrothiophene-3-carbonyl)-7-azaspiro[4.5]decan-10-yl)methyl)pyrimidin-4(3H)-one: Prepared according to General Procedure 3 using 3-((7-azaspiro[4.5]decan-10-yl)methyl)-6-phenylpyrimidin-4(3H)-one (10.0 mg, 0.031 mmol), rac-(3R,4R)-4-phenyltetrahydrothiophene-3-carboxylic acid (7.1 mg, 0.034 mmol), HATU (14.1 mg, 0.037 mmol) and DIPEA (16.2 μL, 0.093 mmol) to give the title compound (13.7 mg, 86%). LCMS (Method B): R_{T}=1.55 min. m/z 514 [M+H]⁺.

Step 2: 3-((7-((3R,4R)-1-Imino-1-oxido-4-phenyltetrahydro-1H-1λ⁶-thiophene-3-carbonyl)-7-azaspiro[4.5]decan-10-yl)methyl)-6-phenylpyrimidin-4(3H)-one and 3-((7-((3S,4S)-1-imino-1-oxido-4-phenyltetrahydro-1H-1λ⁶-thiophene-3-carbonyl)-7-azaspiro[4.5]decan-10-yl)methyl)-6-phenylpyrimidin-4(3H)-one: To a stirring solution of rac-6-phenyl-3-((7-((3R,4R)-4-phenyltetrahydrothiophene-3-carbonyl)-7-azaspiro[4.5]decan-10-yl)methyl)pyrimidin-4(3H)-one (13.7 mg, 0.027 mmol) in MeOH (0.5 mL) was added (diacetoxyiodo)benzene (21.5 mg, 0.067 mmol) and ammonium carbonate (5.1 mg, 0.053 mmol) and the suspension was stirred for 30 min. The mixture was reduced in vacuo. The residue was purified by flash chromatography (0-100% MeOH in EtOAc) and freeze-dried to give the title compound (11.9 mg, 79%). LCMS (Method B): R_{T}=1.19 min, m/z 545 [M+H]⁺. ¹H NMR (500 MHz, DMSO-d₆): δ 8.52-8.37 (m, 1H), 8.10-7.97 (m, 2H), 7.53-7.44 (m, 3H), 7.42-7.18 (m, 5H), 6.98-6.91 (m, 1H), 4.28-3.84 (m, 4H), 3.84-3.30 (m, 5.5H), 3.27-3.08 (m, 1.5H), 2.98-2.65 (m, 1H), 1.90-1.32 (m, 7H), 1.30-1.01 (m, 3H), 1.00-0.80 (m, 1H), 0.76-0.38 (m, 1H).

### Example 13: 6-Phenyl-3-(((S)-7-((R)-2-phenylpiperazine-1-carbonyl)-7-azaspiro[4.5]decan-10-yl)methyl)pyrimidin-4(3H)-one

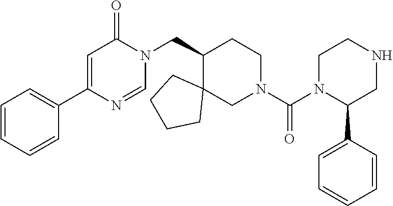 [SMILES: O=C(N1CC[C@H](Cn2cnc(-c3ccccc3)cc2=O)C2(CCCC2)C1)N1CCNC[C@H]1c1ccccc1]

and

### Example 14: 6-Phenyl-3-(((R)-7-((R)-2-phenylpiperazine-1-carbonyl)-7-azaspiro[4.5]decan-10-yl)methyl)pyrimidin-4(3H)-one

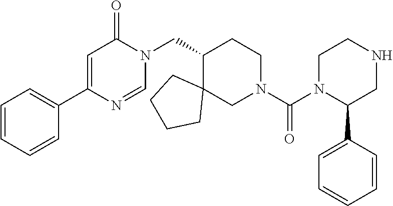 [SMILES: O=C(N1CC[C@@H](Cn2cnc(-c3ccccc3)cc2=O)C2(CCCC2)C1)N1CCNC[C@H]1c1ccccc1]

Step 1: tert-Butyl (3R)-4-[10-[(6-oxo-4-phenyl-pyrimidin-1-yl)methyl]-7-azaspiro[4.5]decane-7-carbonyl]-3-phenyl-piperazine-1-carboxylate: Prepared according to General Procedure 2 using triphosgene (22.9 mg, 0.077 mmol), pyridine (62.3 μL, 0.77 mmol), tert-butyl (R)-3-phenylpiperazine-1-carboxylate (48.7 mg, 0.19 mmol), 3-((7-azaspiro[4.5]decan-10-yl)methyl)-6-phenylpyrimidin-4(3H)-one (50.0 mg, 0.15 mmol) and DIPEA (135 μL, 0.77 mmol) to give the title compound (89.7 mg, 95%). LCMS (Method B): R_{T}=1.64 min, m/z 612 [M+H]⁺.

Step 2: 6-Phenyl-3-(((S)-7-((R)-2-phenylpiperazine-1-carbonyl)-7-azaspiro[4.5]decan-10-yl)methyl)pyrimidin-4(3H)-one and 6-phenyl-3-(((R)-7-((R)-2-phenylpiperazine-1-carbonyl)-7-azaspiro[4.5]decan-10-yl)methyl)pyrimidin-4(3H)-one: Prepared according to General Procedure 1 using tert-butyl (3R)-4-[10-[(6-oxo-4-phenyl-pyrimidin-1-yl)methyl]-7-azaspiro[4.5]decane-7-carbonyl]-3-phenyl-piperazine-1-carboxylate (89.7 mg, 0.15 mmol) and TFA (1.0 mL), stirring for 20 min to give the title compounds as a mixture (76.2 mg, 100%). LCMS (Method B): R_{T}=0.96 min, m/z 512 [M+H]⁺. The material was separated by chiral preparative HPLC using a Lux C1 (21.2 mm×250 mm, 5 μm) column with isocratic solvent conditions: MeOH (0.2% v/v NH₃) and freeze-dried to give: 6-Phenyl-3-(((S)-7-((R)-2-phenylpiperazine-1-carbonyl)-7-azaspiro[4.5]decan-10-yl)methyl)pyrimidin-4(3H)-one (first eluting: 27.1 mg, 35%). LCMS (Method B): R_{T}=0.96 min, m/z 512 [M+H]⁺. ¹H NMR (500 MHz, DMSO-d₆): δ 8.59-8.54 (m, 1H), 8.09-8.03 (m, 2H), 7.53-7.45 (m, 3H), 7.38-7.28 (m, 4H), 7.27-7.17 (m, 1H), 6.98-6.94 (m, 1H), 4.48-4.27 (m, 1H), 4.12-4.04 (dd, 1H), 3.84-3.65 (m, 2H), 3.43-3.34 (m, 1H), 3.28-3.25 (m, 1H), 3.13-2.79 (m, 7H), 2.62-2.55 (m, 1H), 1.92-1.82 (m, 1H), 1.82-1.72 (m, 1H), 1.65-1.41 (m, 4H), 1.41-1.34 (m, 1H), 1.34-1.18 (m, 4H). Chiral purity (Method C): R_{T}=8.08 min, 99.5% ee. 6-Phenyl-3-(((R)-7-((R)-2-phenylpiperazine-1-carbonyl)-7-azaspiro[4.5]decan-10-yl)methyl)pyrimidin-4(3H)-one (second eluting: 27.0 mg, 35%). LCMS (Method B): R_{T}=0.99 min, m/z 512 [M+H]⁺. ¹H NMR (500 MHz, DMSO-d₆): δ 8.55 (s, 1H), 8.08-8.03 (m, 2H), 7.52-7.46 (m, 3H), 7.32-7.24 (m, 4H), 7.21-7.15 (m, 1H), 6.96 (s, 1H), 4.40-4.32 (m, 1H), 4.11 (dd, 1H), 3.79-3.66 (m, 2H), 3.34 (d, 1H), 3.28-3.24 (m, 1H), 3.09-3.02 (m, 1H), 3.01-2.88 (m, 3H), 2.81-2.71 (m, 3H), 2.65 (d, 1H), 1.90-1.79 (m, 2H), 1.68-1.58 (m, 2H), 1.56-1.45 (m, 2H), 1.40-1.20 (m, 5H). Chiral purity (Method C): R_{T}=11.1 min, 99.3% ee.

### Example 15: 4-Phenyl-1-(((S)-7-((R)-2-phenylpiperazine-1-carbonyl)-7-azaspiro[4.5]decan-10-yl)methyl)pyridin-2(1H)-one

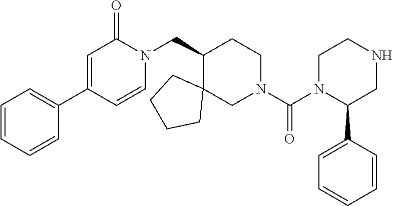 [SMILES: O=C(N1CC[C@H](Cn2ccc(-c3ccccc3)cc2=O)C2(CCCC2)C1)N1CCNC[C@H]1c1ccccc1]

and

### Example 16: 4-Phenyl-1-(((R)-7-((R)-2-phenylpiperazine-1-carbonyl)-7-azaspiro[4.5]decan-10-yl)methyl)pyridin-2(1H)-one

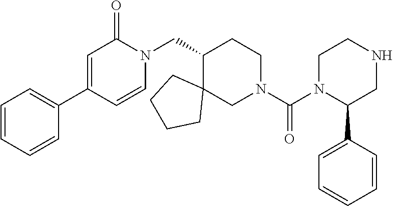 [SMILES: O=C(N1CC[C@@H](Cn2ccc(-c3ccccc3)cc2=O)C2(CCCC2)C1)N1CCNC[C@H]1c1ccccc1]

Step 1: tert-Butyl (3R)-4-(10-((2-oxo-4-phenylpyridin-1(2H)-yl)methyl)-7-azaspiro[4.5]decane-7-carbonyl)-3-phenylpiperazine-1-carboxylate: Prepared according to General Procedure 2 using triphosgene (23.0 mg, 0.078 mmol), pyridine (62.5 μL, 0.78 mmol), tert-butyl (R)-3-phenylpiperazine-1-carboxylate (48.8 mg, 0.19 mmol), 1-((7-azaspiro[4.5]decan-10-yl)methyl)-4-phenylpyridin-2(1H)-one (50.0 mg, 0.16 mmol) and DIPEA (135 μL, 0.78 mmol) to give the title compound (89.7 mg, 95%). LCMS (Method B): RT=1.63 min, m/z 611 [M+H]⁺.

Step 2: 4-Phenyl-1-(((S)-7-((R)-2-phenylpiperazine-1-carbonyl)-7-azaspiro[4.5]decan-10-yl)methyl)pyridin-2(1H)-one and 4-phenyl-1-(((R)-7-((R)-2-phenylpiperazine-1-carbonyl)-7-azaspiro[4.5]decan-10-yl)methyl)pyridin-2(1H)-one: Prepared according to General Procedure 1 using tert-butyl (3R)-4-(10-((2-oxo-4-phenylpyridin-1(2H)-yl)methyl)-7-azaspiro[4.5]decane-7-carbonyl)-3-phenylpiperazine-1-carboxylate (89.7 mg, 0.15 mmol) and TFA (1.0 mL), stirring for 20 min give the mixture of title compounds (70.3 mg, 91%). LCMS (Method B): R_{T}=0.85 min, m/z 511 [M+H]⁺. The material was separated by chiral preparative HPLC using a Lux C1 (21.2 mm×250 mm, 5 μm) column with isocratic solvent conditions: MeOH (0.2% v/v NH₃) and freeze-dried to give: 4-Phenyl-1-(((S)-7-((R)-2-phenylpiperazine-1-carbonyl)-7-azaspiro[4.5]decan-10-yl)methyl)pyridin-2(1H)-one (first eluting: 22.0 mg, 29%). LCMS (Method B): R_{T}=0.97 min, m/z 511 [M+H]⁺. ¹H NMR (500 MHz, DMSO-d₆): δ 7.77-7.69 (m, 3H), 7.51-7.43 (m, 3H), 7.34-7.24 (m, 4H), 7.23-7.17 (m, 1H), 6.67-6.63 (m, 1H), 6.60-6.55 (m, 1H), 4.50-4.23 (m, 1H), 4.07 (dd, 1H), 3.82-3.64 (m, 2H), 3.41-3.33 (m, 1H), 3.13-2.86 (m, 5H), 2.85-2.70 (m, 2H), 2.60 (d, 1H), 1.91-1.82 (m, 1H), 1.82-1.73 (m, 1H), 1.65-1.42 (m, 4.5H), 1.41-1.20 (m, 5.5H). Chiral purity (Method C): R_{T}=5.89 min, 99.5% ee. 4-Phenyl-1-(((R)-7-((R)-2-phenylpiperazine-1-carbonyl)-7-azaspiro[4.5]decan-10-yl)methyl)pyridin-2(1H)-one (second eluting: 18.6 mg, 25%). LCMS (Method B): R_{T}=0.97 min, m/z 511 [M+H]⁺. ¹H NMR (500 MHz, DMSO-d₆): δ 7.75-7.69 (m, 3H), 7.52-7.43 (m, 3H), 7.33-7.24 (m, 4H), 7.23-7.15 (m, 1H), 6.67-6.63 (m, 1H), 6.59-6.54 (m, 1H), 4.60-4.32 (m, 1H), 4.10 (dd, 1H), 3.78-3.64 (m, 2H), 3.35-3.31 (m, 1H), 3.28-3.24 (m, 1H), 3.11-3.02 (m, 1H), 2.99-2.88 (m, 3H), 2.82-2.72 (m, 3H), 2.65 (d, 1H), 1.90-1.80 (m, 2H), 1.67-1.57 (m, 2H), 1.55-1.44 (m, 2H), 1.41-1.19 (m, 5H). Chiral purity (Method C): R_{T}=7.33 min, 99.5% ee.

### Example 17: 1-((1-((2S,4R)-4-Amino-2-phenylpiperidine-1-carbonyl)piperidin-4-yl)methyl)-4-phenylpyridin-2(1H)-one

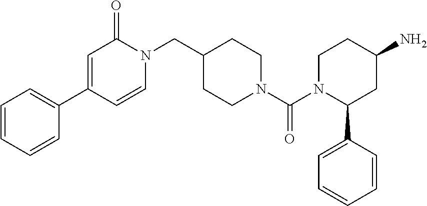 [SMILES: N[C@@H]1CCN(C(=O)N2CCC(Cn3ccc(-c4ccccc4)cc3=O)CC2)[C@H](c2ccccc2)C1]

Step 1: tert-Butyl ((2S,4R)-1-(4-((2-oxo-4-phenylpyridin-1(2H)-yl)methyl)piperidine-1-carbonyl)-2-phenylpiperidin-4-yl)carbamate: Prepared according to General Procedure 2 using triphosgene (11.1 mg, 0.037 mmol), pyridine (30.0 μL, 0.37 mmol), tert-butyl ((2S,4R)-2-phenylpiperidin-4-yl)carbamate (24.7 mg, 0.089 mmol), 4-phenyl-1-(piperidin-4-ylmethyl)pyridin-2(1H)-one (20.0 mg, 0.075 mmol) and DIPEA (65.1 μL, 0.37 mmol) to give the title compound (11.8 mg, 28%). LCMS (Method B): R_{T}=1.40 min, m/z 571 [M+H]⁺.

Step 2: 1-((1-((2S,4R)-4-Amino-2-phenylpiperidine-1-carbonyl)piperidin-4-yl)methyl)-4-phenylpyridin-2(1H)-one: Prepared according to General Procedure 1 using tert-butyl ((2S,4R)-1-(4-((2-oxo-4-phenylpyridin-1(2H)-yl)methyl)piperidine-1-carbonyl)-2-phenylpiperidin-4-yl)carbamate (11.8 mg, 0.021 mmol) and TFA (0.2 mL), stirring for 20 min to give the title compound (9.5 mg, 93%). LCMS (Method B): R_{T}=0.82 min, m/z 471 [M+H]⁺. ¹H NMR (500 MHz, DMSO-d₆): δ 7.74-7.68 (m, 3H), 7.51-7.44 (m, 3H), 7.24 (t, 2H), 7.17 (d, 2H), 7.11 (t, 1H), 6.67 (d, 1H), 6.56 (dd, 1H), 4.03-3.91 (m, 3H), 3.85-3.74 (m, 2H), 3.27-3.20 (m, 2H), 2.88-2.65 (m 3H), 2.03-1.92 (m, 1H), 1.85 (d, 2H), 1.57 (d, 1H), 1.50 (d, 1H), 1.46-1.35 (m, 1H), 1.27 (q, 1H), 1.16-0.93 (m, 2H).

### Example 18: 3-((33-Dimethyl-1-((R)-2-phenylpiperazine-1-carbonyl)piperidin-4-yl)methyl)-6-phenylpyrimidin-4(3H)-one

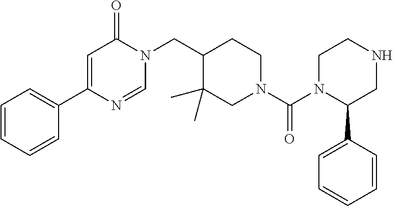 [SMILES: CC1(C)CN(C(=O)N2CCNC[C@H]2c2ccccc2)CCC1Cn1cnc(-c2ccccc2)cc1=O]

Step 1: tert-Butyl 4-(hydroxymethyl)-3,3-dimethyl-piperidine-1-carboxylate: To a stirring solution of tert-butyl 3,3-dimethyl-4-methylene-piperidine-1-carboxylate (500 mg, 2.22 mmol) in anhydrous THF (10 mL) under a N₂ atmosphere at 0° C. was added borane-THF complex (5.6 mL, 5.55 mmol) dropwise. The mixture was stirred while warming to rt for 24 h. The solution was heated to 50° C. and stirring continued for 5 d. 5 M sodium acetate (aq) solution (1.5 mL, 7.69 mmol) was added followed by hydrogen peroxide (30% w/w in water) (1.5 mL, 15.1 mmol). Stirring was continued for 16 h. The mixture was partitioned between EtOAc (10 mL) and saturated sodium hydrogen carbonate (aq) solution (10 mL). The aqueous was separated and extracted with EtOAc (2×5 mL). The combined EtOAc fractions were dried (Na₂SO₄), filtered and reduced in vacuo. The residue was purified by flash chromatography (0-100% EtOAc in cyclohexane) to give the title compound (502 mg, 93%). ¹H NMR (500 MHz, CDCl₃): δ 4.30-4.01 (m, 1H), 3.84 (d, 1H), 3.76-3.50 (m, 1H), 3.35 (dd, 1H), 2.79-2.60 (m, 1H), 2.58-2.38 (m, 1H), 1.79-1.67 (m, 1H), 1.45 (s, 9H), 1.43-1.32 (2H), 1.23-1.14 (m, 1H), 0.97 (s, 3H), 0.81 (s, 3H).

Step 2: tert-Butyl 4-(bromomethyl)-3,3-dimethyl-piperidine-1-carboxylate: To a stirring solution of tert-butyl 4-(hydroxymethyl)-3,3-dimethyl-piperidine-1-carboxylate (502 mg, 2.06 mmol) and triphenylphosphine (704 mg, 2.68 mmol) in anhydrous DCM (15 mL) at 0° C. was added carbon tetrabromide (890 mg, 2.68 mmol). After stirring at rt for 68 h, the mixture was cooled to 0° C. then further triphenylphosphine (704 mg, 2.68 mmol) and carbon tetrabromide (890 mg, 2.68 mmol) were added. Stirring was continued while warming to rt for 20 h. The solvent was removed in vacuo. The residue was purified by flash chromatography (0-20% EtOAc in cyclohexane) to give the title compound (93.3 mg, 15%). ¹H NMR (500 MHz, CDCl₃): δ 4.35-3.97 (m, 1H), 3.84-3.52 (m, 2H), 3.03 (t, 1H), 2.80-2.58 (m, 1H), 2.57-2.33 (m, 1H), 2.04-1.96 (m, 1H), 1.60 (tt, 1H), 1.45 (s, 9H), 1.36-1.29 (m, 1H), 0.98 (s, 3H), 0.80 (s, 3H).

Step 3: tert-Butyl 3,3-dimethyl-4-((6-oxo-4-phenylpyrimidin-1(6H)-yl)methyl)piperidine-1-carboxylate: A stirring solution of 6-phenylpyrimidin-4(3H)-one (25.4 mg, 0.15 mmol), tert-butyl 4-(bromomethyl)-3,3-dimethyl-piperidine-1-carboxylate (45.1 mg, 0.15 mmol) and cesium carbonate (96.0 mg, 0.29 mmol) in anhydrous 1,4-dioxane (1 mL) was heated at 120° C. for 68 h. The mixture was partitioned between DCM (6 mL) and 1:1 brine/water (12 mL). The aqueous was separated and extracted with DCM (3×3 mL). The combined DCM fractions were dried (phase separator), filtered and reduced in vacuo. The residue was purified by flash chromatography (0-50% EtOAc in cyclohexane) to give the title compound (31.8 mg, 21%). LCMS (Method B): R_{T}=1.45 min, m/z 342 [M−butene+H]⁺.

Step 4: 3-((3,3-Dimethylpiperidin-4-yl)methyl)-6-phenylpyrimidin-4(3H)-one: Prepared according to General Procedure 1 using tert-butyl 3,3-dimethyl-4-((6-oxo-4-phenylpyrimidin-1(6H)-yl)methyl)piperidine-1-carboxylate (31.8 mg, 0.080 mmol) and TFA (0.2 mL), stirring for 20 min to give the title compound (22.5 mg, 95%). LCMS (Method B): R_{T}=0.65 min, m/z 298 [M+H]⁺.

Step 5: tert-Butyl (3R)-4-(3,3-dimethyl-4-((6-oxo-4-phenylpyrimidin-1(6H)-yl)methyl)piperidine-1-carbonyl)-3-phenylpiperazine-1-carboxylate: Prepared according to General Procedure 2 using triphosgene (6.0 mg, 0.020 mmol), pyridine (16.3 μL, 0.20 mmol), tert-butyl (R)-3-phenylpiperazine-1-carboxylate (12.7 mg, 0.048 mmol), 3-((3,3-dimethylpiperidin-4-yl)methyl)-6-phenylpyrimidin-4(3H)-one (12.0 mg, 0.040 mmol) and DIPEA (35.2 μL, 0.20 mmol) to give the title compound (23.8 mg, 99%). LCMS (Method B): R_{T}=1.56 min, m/z 586 [M+H]⁺.

Step 6: 3-((3,3-Dimethyl-1-((R)-2-phenylpiperazine-1-carbonyl)piperidin-4-yl)methyl)-6-phenylpyrimidin-4(3H)-one: Prepared according to General Procedure 1 using tert-butyl (3R)-4-(3,3-dimethyl-4-((6-oxo-4-phenylpyrimidin-1(6H)-yl)methyl)piperidine-1-carbonyl)-3-phenylpiperazine-1-carboxylate (23.8 mg, 0.04 mmol) and TFA (0.3 mL), stirring for 20 min to give the title compound (19.6 mg, 99%). LCMS (Method B): R_{T}=0.85 min, m/z 486 [M+H]⁺. ¹H NMR (500 MHz, DMSO-d₆): δ 8.55 (s, 0.5H), 8.51 (s, 0.5H), 8.09-8.03 (m, 2H), 7.52-7.47 (m, 3H), 7.40-7.22 (m, 6H), 6.97-6.94 (m, 1H), 4.49 (dd, 0.5H), 4.42 (dd, 0.5H), 4.20-4.10 (m, 1H), 4.02 (d, 0.5H), 3.89 (d, 0.5H), 3.62-3.53 (m, 1H), 3.49-3.41 (m, 1H), 3.26-3.05 (m, 6H), 2.80-2.70 (m, 0.5H), 2.63-2.56 (m, 1H), 2.47-2.44 (m, 0.5H), 1.80-1.70 (m, 1H), 1.44-1.30 (m, 1H), 1.28-1.18 (m, 1H), 1.02 (s, 1.5H), 0.96 (s, 1.5H), 0.81 (s, 1.5H), 0.59 (s, 1.5H).

### Example 19: 3-((1-((2S,4R)-4-Amino-2-phenylpiperidine-1-carbonyl)-3,3-dimethylpiperidin-4-yl)methyl)-6-phenylpyrimidin-4(3H)-one

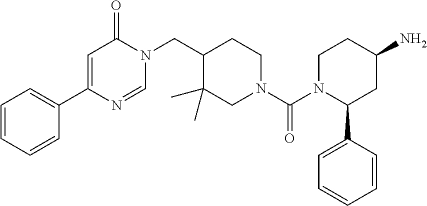 [SMILES: CC1(C)CN(C(=O)N2CC[C@@H](N)C[C@H]2c2ccccc2)CCC1Cn1cnc(-c2ccccc2)cc1=O]

Step 1: tert-Butyl ((2S,4R)-1-(3,3-dimethyl-4-((6-oxo-4-phenylpyrimidin-1(6H)-yl)methyl)piperidine-1-carbonyl)-2-phenylpiperidin-4-yl)carbamate: Prepared according to General Procedure 2 using triphosgene (5.3 mg, 0.018 mmol), pyridine (14.4 μL, 0.18 mmol), tert-butyl ((2S,4R)-2-phenylpiperidin-4-yl)carbamate (11.8 mg, 0.043 mmol), 3-((3,3-dimethylpiperidin-4-yl)methyl)-6-phenylpyrimidin-4(3H)-one (10.6 mg, 0.036 mmol) and DIPEA (31.1 μL, 0.18 mmol) to give the title compound (19.5 mg, 91%). LCMS (Method B): R_{T}=1.51 min, m/z 600 [M+H]⁺.

Step 2: 3-((1-((2S,4R)-4-Amino-2-phenylpiperidine-1-carbonyl)-3,3-dimethylpiperidin-4-yl)methyl)-6-phenylpyrimidin-4(3H)-one: Prepared according to General Procedure using tert-butyl ((2S,4R)-1-(3,3-dimethyl-4-((6-oxo-4-phenylpyrimidin-1(6H)- yl)methyl)piperidine-1-carbonyl)-2-phenylpiperidin-4-yl)carbamate (19.5 mg, 0.033 mmol) and TFA (0.3 mL), stirring for 20 min to give the title compound (14.7 mg, 90%). LCMS (Method B): R_{T}=0.84 min, m/z 500 [M+H]⁺. ¹H NMR (500 MHz, DMSO-d₆): δ 8.59-8.51 (m, 1H), 8.11-8.01 (m, 2H), 7.55-7.44 (m, 3H), 7.29-7.24 (m, 1H), 7.24-7.17 (m, 3H), 7.17-7.10 (m, 1H), 6.97-6.93 (m, 1H), 4.21-4.09 (m, 1.5H), 4.00 (d, 0.5H), 3.96-3.87 (m, 1H), 3.60-3.49 (m, 2H), 3.26-3.22 (m, 0.5H), 3.14 (dt, 0.5H), 2.80-2.66 (m, 1.5H), 2.66-2.53 (m, 2H), 2.40-2.34 (m, 0.5H), 2.11-1.56 (m, 5H), 1.44-1.16 (m, 4H), 1.01 (s, 1.5H), 0.95 (s, 1.5H), 0.82 (s, 1.5H), 0.49 (s, 1.5H).

### Example 20: 1-((33-Dimethyl-1-((R)-2-phenylpiperazine-1-carbonyl)piperidin-4-yl)methyl)-4-phenylpyridin-2(1H)-one

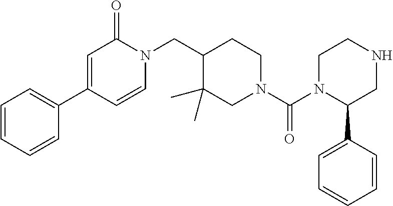 [SMILES: CC1(C)CN(C(=O)N2CCNC[C@H]2c2ccccc2)CCC1Cn1ccc(-c2ccccc2)cc1=O]

Step 1: tert-Butyl 3,3-dimethyl-4-((2-oxo-4-phenylpyridin-1(2H)-yl)methyl)piperidine-1-carboxylate: A stirring solution of 4-phenylpyridin-2(1H)-one (23.9 mg, 0.14 mmol), tert-butyl 4-(bromomethyl)-3,3-dimethyl-piperidine-1-carboxylate (42.7 mg, 0.14 mmol) and cesium carbonate (90.9 mg, 0.28 mmol) in anhydrous 1,4-dioxane (1 mL) was heated at 120° C. for 68 h. The mixture was partitioned between DCM (6 mL) and 1:1 brine/water (12 mL). The aqueous was separated and extracted with DCM (3×3 mL). The combined DCM fractions were dried (phase separator), filtered and reduced in vacuo. The residue was purified by flash chromatography (0-50% EtOAc in cyclohexane) to give the title compound (31.9 mg, 58%). LCMS (Method B): R_{T}=1.45 min, m/z 341 [M−butene+H]⁺.

Step 2: 1-((3,3-Dimethylpiperidin-4-yl)methyl)-4-phenylpyridin-2(1H)-one: Prepared according to General Procedure 1 using tert-butyl 3,3-dimethyl-4-((2-oxo-4-phenylpyridin-1(2H)-yl)methyl)piperidine-1-carboxylate (31.9 mg, 0.080 mmol) and TFA (0.2 mL), stirring for 20 min to give the title compound (21.6 mg, 91%). LCMS (Method B): R_{T}=0.65 min, m/z 297 [M+H]⁺.

Step 3: tert-Butyl (3R)-4-(3,3-dimethyl-4-((2-oxo-4-phenylpyridin-1(2H)-yl)methyl)piperidine-1-carbonyl)-3-phenylpiperazine-1-carboxylate: Prepared according to General Procedure 2 using triphosgene (5.0 mg, 0.017 mmol), pyridine (13.5 μL, 0.17 mmol), tert-butyl (R)-3-phenylpiperazine-1-carboxylate (10.5 mg, 0.040 mmol), 1-((3,3-dimethylpiperidin-4-yl)methyl)-4-phenylpyridin-2(1H)-one (9.9 mg, 0.033 mmol) and DIPEA (29.2 μL, 0.17 mmol) to give the title compound (17.7 mg, 91%). LCMS (Method B): R_{T}=1.54 min, m/z 585 [M+H]⁺.

Step 4: 1-((3,3-Dimethyl-1-((R)-2-phenylpiperazine-1-carbonyl)piperidin-4-yl)methyl)-4-phenylpyridin-2(1H)-one: Prepared according to General Procedure 1 using tert-butyl (3R)-4-(3,3-dimethyl-4-((2-oxo-4-phenylpyridin-1(2H)-yl)methyl)piperidine-1-carbonyl)-3-phenylpiperazine-1-carboxylate (17.7 mg, 0.030 mmol) and TFA (0.2 mL), stirring for 20 min to give the title compound (13.1 mg, 82%). LCMS (Method B): R_{T}=0.84 min, m/z 485 [M+H]⁺. ¹H NMR (500 MHz, DMSO-d₆): δ 7.74-7.67 (m, 3H), 7.53-7.42 (m, 3H), 7.34-7.24 (m, 4H), 7.24-7.16 (m, 1H), 6.67-6.62 (m, 1H), 6.59-6.53 (m, 1H), 4.44-4.39 (m, 0.5H), 4.33-4.27 (m, 0.5H), 4.19-4.09 (m, 1H), 3.92-3.77 (m, 1H), 3.63-3.53 (m, 1H), 3.40-3.32 (m, 1H), 3.11-3.05 (m, 0.5H), 3.05-2.97 (m, 2H), 2.97-2.92 (m, 1H), 2.86-2.75 (m, 2H), 2.71 (td, 0.5H), 2.62-2.53 (m, 1H), 2.48-2.42 (m, 1H), 1.78-1.69 (m, 1H), 1.47-1.36 (m, 1H), 1.29-1.17 (m, 1H), 1.01 (s, 1.5H), 0.96 (s, 1.5H), 0.82 (s, 1.5H), 0.70 (s, 1.5H).

### Example 21: 1-((1-((2S,4R)-4-Amino-2-phenylpiperidine-1-carbonyl)-3,3-dimethylpiperidin-4-yl)methyl)-4-phenylpyridin-2(1H)-one

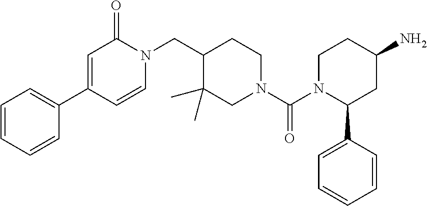 [SMILES: CC1(C)CN(C(=O)N2CC[C@@H](N)C[C@H]2c2ccccc2)CCC1Cn1ccc(-c2ccccc2)cc1=O]

Step 1: tert-Butyl ((2S,4R)-1-(3,3-dimethyl-4-((2-oxo-4-phenylpyridin-1(2H)-yl)methyl)piperidine-1-carbonyl)-2-phenylpiperidin-4-yl)carbamate: Prepared according to General Procedure 2 using triphosgene (5.9 mg, 0.020 mmol), pyridine (15.9 μL, 0.20 mmol), tert-butyl ((2S,4R)-2-phenylpiperidin-4-yl)carbamate (13.1 mg, 0.047 mmol), 1-((3,3-dimethylpiperidin-4-yl)methyl)-4-phenylpyridin-2(1H)-one (11.7 mg, 0.040 mmol) and DIPEA (34.5 μL, 0.20 mmol) to give the title compound (16.2 mg, 69%). LCMS (Method B): R_{T}=1.49 min, m/z 599 [M+H]⁺.

Step 2: 1-((1-((2S,4R)-4-Amino-2-phenylpiperidine-1-carbonyl)-3,3-dimethylpiperidin-4-yl)methyl)-4-phenylpyridin-2(1H)-one: Prepared according to General Procedure 1 using tert-butyl ((2S,4R)-1-(3,3-dimethyl-4-((2-oxo-4-phenylpyridin-1(2H)-yl)methyl)piperidine-1-carbonyl)-2-phenylpiperidin-4-yl)carbamate (16.2 mg, 0.027 mmol) and TFA (0.2 mL), stirring for 20 min to give the title compound (12.9 mg, 91% yield). LCMS (Method B): R_{T}=0.85 min, m/z 499 [M+H]⁺. ¹H NMR (500 MHz, DMSO-d₆): δ 7.75-7.67 (m, 3H), 7.52-7.43 (m, 3H), 7.29-7.24 (m, 1H), 7.24-7.10 (m, 4H), 6.68-6.62 (m, 1H), 6.60-6.54 (m, 1H), 4.19-4.07 (m, 1.5H), 4.00 (d, 0.5H), 3.97-3.88 (m, 1H), 3.62-3.47 (m, 2H), 3.26-3.23 (m, 0.5H), 3.15 (dt, 0.5H), 2.81-2.70 (m, 1.5H), 2.69-2.54 (m, 2H), 2.43-2.37 (m, 0.5H), 1.87-1.70 (m, 3H), 1.45-1.19 (m, 4H), 1.00 (s, 1.5H), 0.94 (s, 1.5H), 0.82 (s, 1.5H), 0.50 (s, 1.5H).

### Example 22: (R)-1-((1-(2-(2,5-Difluorophenyl)piperazine-1-carbonyl)piperidin-4-yl)methyl)-4-phenylpyridin-2(1H)-one

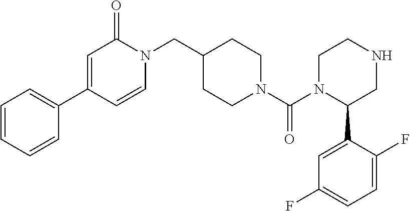 [SMILES: O=C(N1CCC(Cn2ccc(-c3ccccc3)cc2=O)CC1)N1CCNC[C@H]1c1cc(F)ccc1F]

Step 1: tert-Butyl (R)-3-(2,5-difluorophenyl)-4-(4-((2-oxo-4-phenylpyridin-1(2H)-yl)methyl)piperidine-1-carbonyl)piperazine-1-carboxylate: Prepared according to General Procedure 2 using triphosgene (5.5 mg, 0.019 mmol), pyridine (15.0 μL, 0.19 mmol), tert-butyl (R)-3-(2,5-difluorophenyl)piperazine-1-carboxylate (13.3 mg, 0.045 mmol), 4-phenyl-1-(piperidin-4-ylmethyl)pyridin-2(1H)-one (10.0 mg, 0.037 mmol) and DIPEA (32.5 μL, 0.19 mmol) to give the title compound (12.1 mg, 55%). LCMS (Method B): R_{T}=1.48 min, m/z 593 [M+H]⁺.

Step 2: (R)-1-((1-(2-(2,5-Difluorophenyl)piperazine-1-carbonyl)piperidin-4-yl)methyl)-4-phenylpyridin-2(1H)-one: Prepared according to General Procedure 1 using tert-butyl (R)-3-(2,5-difluorophenyl)-4-(4-((2-oxo-4-phenylpyridin-1(2H)-yl)methyl)piperidine-1-carbonyl)piperazine-1-carboxylate (12.1 mg, 0.020 mmol) and TFA (0.2 mL), stirring for 20 min to give the title compound (8.9 mg, 87%). LCMS (Method B): R_{T}=0.82 min, m/z 493 [M+H]⁺. ¹H NMR (500 MHz, DMSO-d₆): δ 7.74-7.67 (m, 3H), 7.52-7.44 (m, 3H), 7.22 (ddd, 1H), 7.16 (td, 1H), 7.10-7.02 (m, 1H), 6.66 (d, 1H), 6.56 (dd, 1H), 4.49 (dd, 1H), 3.88-3.73 (m, 4H), 3.17 (dt, 1H), 2.92 (dd, 1H), 2.86 (dd, 1H), 2.84-2.69 (m, 4H), 2.69-2.63 (m, 1H), 2.04-1.93 (m, 1H), 1.58 (d, 1H), 1.50 (d, 1H), 1.18-1.02 (m, 2H).

### Example 23: 1-(((R)-7-((2S,4R)-4-Amino-2-phenylpiperidine-1-carbonyl)-7-azaspiro[4.5]decan-10-yl)methyl)-4-phenylpyridin-2(1H)-one

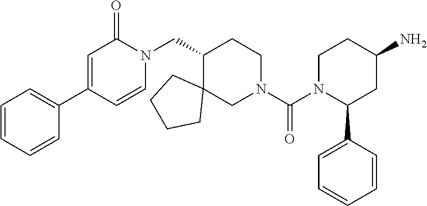 [SMILES: N[C@@H]1CCN(C(=O)N2CC[C@@H](Cn3ccc(-c4ccccc4)cc3=O)C3(CCCC3)C2)[C@H](c2ccccc2)C1]

Step 1: tert-Butyl ((2S,4R)-1-((R)-10-((2-oxo-4-phenylpyridin-1(2H)-yl)methyl)-7-azaspiro[4.5]decane-7-carbonyl)-2-phenylpiperidin-4-yl)carbamate and tert-butyl ((2S,4R)-1-((S)-10-((2-oxo-4-phenylpyridin-1(2H)-yl)methyl)-7-azaspiro[4.5]decane-7-carbonyl)-2-phenylpiperidin-4-yl)carbamate: Prepared according to General Procedure 2 using triphosgene (23.0 mg, 0.078 mmol), pyridine (62.5 μL, 0.78 mmol), tert-butyl ((2S,4R)-2-phenylpiperidin-4-yl)carbamate (51.4 mg, 0.19 mmol), 1-((7-azaspiro[4.5]decan-10-yl)methyl)-4-phenylpyridin-2(1H)-one (50.0 mg, 0.16 mmol) and DIPEA (135 μL, 0.78 mmol) to give the mixture of title compounds. The material was purified by chiral SFC using a Lux C1 (21.2 mm×250 mm, 5 μm) column with isocratic solvent conditions: 60:40 MeOH/CO₂ to give:

tert-Butyl ((2S,4R)-1-((R)-10-((2-oxo-4-phenylpyridin-1(2H)-yl)methyl)-7-azaspiro[4.5]decane-7-carbonyl)-2-phenylpiperidin-4-yl)carbamate (second eluting: 33.5 mg, 34%). LCMS (Method B): R_{T}=1.61 min, m/z 625 [M+H]⁺. ¹H NMR (500 MHz, CDCl₃): δ 7.59-7.55 (m, 2H), 7.48-7.41 (m, 3H), 7.26-7.23 (m, 4H), 7.22 (d, 1H), 7.20-7.14 (m, 1H), 6.77 (d, 1H), 6.43 (dd, 1H), 4.36 (br s, 1H), 4.18 (dd, 1H), 4.10 (d, 1H), 3.97 (dt, 1H), 3.84-3.72 (m, 1H), 3.71-3.59 (m, 1H), 3.56-3.45 (m, 2H), 3.28 (dt, 1H), 3.02-2.84 (m, 2H), 2.72 (d, 1H), 2.14 (d, 1H), 2.07 (d, 1H), 1.91 (tt, 1H), 1.86-1.75 (m, 1H), 1.74-1.65 (m, 2H), 1.65-1.58 (m, 1H), 1.54-1.45 (m, 4H), 1.42 (s, 9H), 1.35-1.21 (m, 3H). Chiral purity (Method D): R_{T}=3.27 min, 100% ee.

tert-Butyl ((2S,4R)-1-((S)-10-((2-oxo-4-phenylpyridin-1(2H)-yl)methyl)-7-azaspiro[4.5]decane-7-carbonyl)-2-phenylpiperidin-4-yl)carbamate (first eluting: 30.8 mg, 32%). LCMS (Method B): R_{T}=1.60 min, m/z 625 [M+H]⁺. ¹H NMR (500 MHz, CDCl₃): δ 7.60-7.55 (m, 2H), 7.49-7.43 (m, 3H), 7.31-7.26 (m, 4H), 7.24 (d, 1H), 7.21-7.17 (m, 1H), 6.78 (d, 1H), 6.44 (dd, 1H), 4.35 (br s, 1H), 4.15 (dd, 1H), 4.09 (dd, 1H), 4.01 (d, 1H), 3.80-3.64 (m, 2H), 3.60 (d, 1H), 3.21 (dt, 1H), 3.02-2.88 (m, 2H), 2.61 (d, 1H), 2.15 (d, 1H), 2.06 (d, 1H), 1.93 (tt, 1H), 1.84-1.75 (m, 1H), 1.73-1.63 (m, 1H), 1.53-1.44 (m, 5H), 1.41 (s, 9H), 1.40-1.23 (m, 3H), 1.22-1.06 (m, 2H). Chiral purity (Method D): R_{T}=1.56 min, 100% ee.

Step 2: 1-(((R)-7-((2S,4R)-4-Amino-2-phenylpiperidine-1-carbonyl)-7-azaspiro[4.5]decan-10-yl)methyl)-4-phenylpyridin-2(1H)-one: Prepared according to General Procedure 1 using tert-butyl ((2S,4R)-1-((R)-10-((2-oxo-4-phenylpyridin-1(2H)-yl)methyl)-7-azaspiro[4.5]decane-7-carbonyl)-2-phenylpiperidin-4-yl)carbamate (33.5 mg, 0.054 mmol) and TFA (0.3 mL), stirring for 20 min to give the title compound (28.1 mg, 98%). LCMS (Method B): R_{T}=0.89 min, m/z 525 [M+H]⁺. ¹H NMR (500 MHz, DMSO-d₆): δ 7.76-7.68 (m, 3H), 7.51-7.42 (m, 3H), 7.22 (t, 2H), 7.18 (d, 2H), 7.14 (t, 1H), 6.65 (s, 1H), 6.57 (dd, 1H), 4.08 (dd, 1H), 3.95 (dd, 1H), 3.91-3.82 (m, 1H), 3.72 (t, 1H), 3.41 (d, 1H), 3.25-3.20 (m, 1H), 2.95-2.77 (m, 2H), 2.72-2.64 (m, 2H), 1.92-1.72 (m, 4H), 1.70-1.57 (m, 2H), 1.57-1.40 (m, 3H), 1.39-1.25 (m, 4H), 1.25-1.12 (m, 2H).

### Example 24: 1-(((S)-7-((2S,4R)-4-Amino-2-phenylpiperidine-1-carbonyl)-7-azaspiro[4.5]decan-10-yl)methyl)-4-phenylpyridin-2(1H)-one

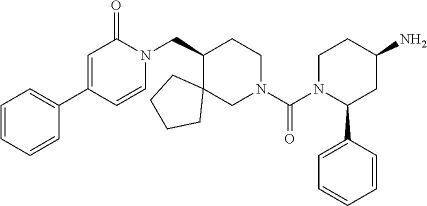 [SMILES: N[C@@H]1CCN(C(=O)N2CC[C@H](Cn3ccc(-c4ccccc4)cc3=O)C3(CCCC3)C2)[C@H](c2ccccc2)C1]

Prepared according to General Procedure 1 using tert-butyl ((2S,4R)-1-((S)-10-((2-oxo-4-phenylpyridin-1(2H)-yl)methyl)-7-azaspiro[4.5]decane-7-carbonyl)-2-phenylpiperidin-4-yl)carbamate (30.8 mg, 0.049 mmol) and TFA (0.3 mL), stirring for 20 min to give the title compound (25.5 mg, 98%). LCMS (Method B): R_{T}=0.92 min, m/z 525 [M+H]⁺. ¹H NMR (500 MHz, DMSO-d₆): δ 7.81-7.68 (m, 3H), 7.59-7.40 (m, 3H), 7.31-7.23 (m, 2H), 7.23-7.12 (m, 3H), 6.66 (s, 1H), 6.58 (d, 1H), 4.06 (d, 1H), 4.00-3.86 (m, 2H), 3.70 (t, 1H), 3.49 (d, 1H), 3.16 (d, 1H), 2.98 (t, 1H), 2.78-2.70 (m, 1H), 2.66-2.56 (m, 3H), 1.92-1.72 (m, 4H), 1.64-1.54 (m, 1H), 1.53-1.43 (m, 2H), 1.42-1.13 (m, 8H), 1.08-0.96 (m, 1H).

### Example 25: 3-(((R)-7-((2S,4R)-4-Amino-2-phenylpiperidine-1-carbonyl)-7-azaspiro[4.5]decan-10-yl)methyl)-6-phenylpyrimidin-4(3H)-one

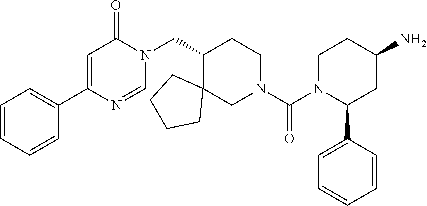 [SMILES: N[C@@H]1CCN(C(=O)N2CC[C@@H](Cn3cnc(-c4ccccc4)cc3=O)C3(CCCC3)C2)[C@H](c2ccccc2)C1]

Step 1: tert-Butyl ((2S,4R)-1-((R)-10-((6-oxo-4-phenylpyrimidin-1(6H)-yl)methyl)-7-azaspiro[4.5]decane-7-carbonyl)-2-phenylpiperidin-4-yl)carbamate and tert-butyl ((2S,4R)-1-((S)-10-((6-oxo-4-phenylpyrimidin-1(6H)-yl)methyl)-7-azaspiro[4.5]decane-7-carbonyl)-2-phenylpiperidin-4-yl)carbamate: Prepared according to General Procedure 2 using triphosgene (22.9 mg, 0.077 mmol), pyridine (62.3 μL, 0.77 mmol), tert-butyl ((2S,4R)-2-phenylpiperidin-4-yl)carbamate (51.3 mg, 0.19 mmol), 3-((7-azaspiro[4.5]decan-10-yl)methyl)-6-phenylpyrimidin-4(3H)-one (50.0 mg, 0.15 mmol) and DIPEA (135 μL, 0.77 mmol) to give the mixture of title compounds. The material was purified by chiral SFC using a Lux C4 (21.2 mm×250 mm, 5 μm) column with isocratic solvent conditions: 50:50 MeOH/CO₂ to give:

tert-Butyl ((2S,4R)-1-((R)-10-((6-oxo-4-phenylpyrimidin-1(6H)-yl)methyl)-7-azaspiro[4.5]decane-7-carbonyl)-2-phenylpiperidin-4-yl)carbamate (first eluted: 29.3 mg, 30%). LCMS (Method B): R_{T}=1.63 min, m/z 626 [M+H]⁺. ¹H NMR (500 MHz, CDCl₃): δ 8.07 (s, 1H), 7.97-7.91 (m, 2H), 7.51-7.44 (m, 3H), 7.26-7.23 (m, 4H), 7.21-7.12 (m, 1H), 6.86 (s, 1H), 4.36 (br s, 1H), 4.22-4.14 (m, 2H), 3.98 (dt, 1H), 3.74-3.46 (m, 3H), 3.27 (dt, 1H), 3.00-2.84 (m, 2H), 2.68 (d, 1H), 2.14 (d, 1H), 2.08 (d, 1H), 1.90 (tt, 1H), 1.86-1.78 (m, 1H), 1.77-1.67 (m, 2H), 1.67-1.57 (m, 1H), 1.53-1.43 (m, 5H), 1.42 (s, 9H), 1.35-1.23 (m, 3H). Chiral purity (Method E): R_{T}=2.25 min, 100% ee.

tert-Butyl ((2S,4R)-1-((S)-10-((6-oxo-4-phenylpyrimidin-1(6H)-yl)methyl)-7-azaspiro[4.5]decane-7-carbonyl)-2-phenylpiperidin-4-yl)carbamate (second eluted: 27.1 mg, 27%). LCMS (Method B): R_{T}=1.62 min, m/z 626 [M+H]⁺. ¹H NMR (500 MHz, CDCl₃): δ 8.09 (s, 1H), 7.98-7.92 (m, 2H), 7.50-7.45 (m, 3H), 7.31-7.26 (m, 4H), 7.22-7.17 (m, 1H), 6.86 (s, 1H), 4.36 (br s, 1H), 4.21-4.12 (m, 2H), 4.04 (d, 1H), 3.76-3.53 (m, 3H), 3.20 (dt, 1H), 2.94 (t, 2H), 2.56 (d, 1H), 2.15 (d, 1H), 2.07 (d, 1H), 1.91 (tt, 1H), 1.85-1.75 (m, 1H), 1.75-1.62 (m, 1H), 1.59-1.55 (m, 1H), 1.52-1.44 (m, 4H), 1.41 (s, 9H), 1.37-1.24 (m, 3H) 1.17-1.06 (m, 2H). Chiral purity (Method E): R_{T}=3.28 min, 99.8% ee.

Step 2: 3-(((R)-7-((2S,4R)-4-Amino-2-phenylpiperidine-1-carbonyl)-7-azaspiro[4.5]decan-10-yl)methyl)-6-phenylpyrimidin-4(3H)-one: Prepared according to General Procedure using tert-butyl ((2S,4R)-1-((R)-10-((6-oxo-4-phenylpyrimidin-1(6H)- yl)methyl)-7-azaspiro[4.5]decane-7-carbonyl)-2-phenylpiperidin-4-yl)carbamate (29.3 mg, 0.047 mmol) and TFA (0.3 mL), stirring for 20 min to give the title compound (23.3 mg, 94%). LCMS (Method B): R_{T}=0.90 min, m/z 526 [M+H]⁺. ¹H NMR (500 MHz, DMSO-d₆): δ 8.54 (s, 1H), 8.09-8.02 (m, 2H), 7.54-7.45 (m, 3H), 7.22-7.16 (m, 4H), 7.13 (t, 1H), 6.96 (s, 1H), 4.10 (dd, 1H), 3.93 (dd, 1H), 3.88 (dt, 1H), 3.71 (t, 1H), 3.44 (d, 1H), 3.22 (dt, 1H), 2.93-2.79 (m, 1H), 2.73 (tt, 1H), 2.69-2.60 (m, 2H), 2.27 (br s, 2H), 1.90-1.72 (m, 4H), 1.71-1.58 (m, 2H), 1.58-1.44 (m, 2H), 1.42-1.15 (m, 7H).

### Example 26: 3-(((S)-7-((2S,4R)-4-Amino-2-phenylpiperidine-1-carbonyl)-7-azaspiro[4.5]decan-10-yl)methyl)-6-phenylpyrimidin-4(3H)-one

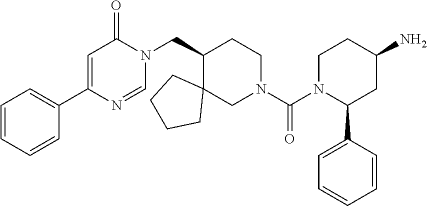 [SMILES: N[C@@H]1CCN(C(=O)N2CC[C@H](Cn3cnc(-c4ccccc4)cc3=O)C3(CCCC3)C2)[C@H](c2ccccc2)C1]

Prepared according to General Procedure 1 using tert-butyl ((2S,4R)-1-((S)-10-((6-oxo-4-phenylpyrimidin-1(6H)-yl)methyl)-7-azaspiro[4.5]decane-7-carbonyl)-2-phenylpiperidin-4-yl)carbamate (27.1 mg, 0.043 mmol) and TFA (0.3 mL), stirring for 20 min to give the title compound (22.1 mg, 96%). LCMS (Method B): R_{T}=0.92 min, m/z 526 [M+H]⁺. ¹H NMR (500 MHz, DMSO-d₆): δ 8.58 (s, 1H), 8.10-8.04 (m, 2H), 7.53-7.46 (m, 3H), 7.26 (t, 2H), 7.19 (d, 2H), 7.15 (t, 1H), 6.97 (s, 1H), 4.08 (dd, 1H), 3.96 (d, 1H), 3.91 (dd, 1H), 3.70 (t, 1H), 3.52 (d, 1H), 3.15 (dt, 1H), 2.97 (t, 1H), 2.73 (tt, 1H), 2.62 (t, 1H), 2.17 (br s, 2H), 1.91-1.72 (m, 4H), 1.65-1.54 (m, 1H), 1.53-1.43 (m, 2H), 1.43-1.11 (m, 8H), 1.08-0.96 (m, 1H).

### Example 27: 3-((7-((2S,4R)-4-Amino-2-phenylpiperidine-1-carbonyl)-7-azaspiro[4.5]decan-10-yl)methyl)-6-fluoroauinazolin-4(3H)-one

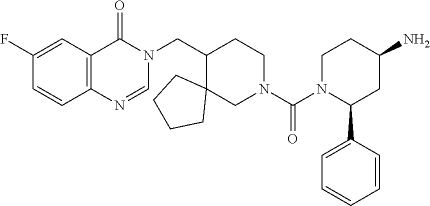 [SMILES: N[C@@H]1CCN(C(=O)N2CCC(Cn3cnc4ccc(F)cc4c3=O)C3(CCCC3)C2)[C@H](c2ccccc2)C1]

Step 1: tert-Butyl 10-(iodomethyl)-7-azaspiro[4.5]decane-7-carboxylate: To tert-butyl 10-(hydroxymethyl)-7-azaspiro[4.5]decane-7-carboxylate (36.0 g, 134 mmol) in THF (600 mL) were added triphenylphosphine (45.6 g, 174 mmol) and trimethylamine (20.0 g, 174 mmol), followed by iodine (44.2 g, 174 mmol). After 24 h, the reaction mixture was treated with water and extracted with MTBE. The organic phase was decanted, washed with saturated sodium thiosulfate (aq) solution, dried (Na₂SO₄) and evaporated to dryness to give the title compound (41.2 g, 81%). ¹H NMR (400 MHz, CDCl₃): δ 4.03-3.78 (m, 1H), 3.68-3.48 (m, 2H), 3.12 (t, 1H), 2.90 (t, 1H), 2.60 (d, 1H), 2.02-1.95 (m, 1H), 1.79 (m, 1H), 1.74-1.40 (m, 6H), 1.44 (s, 9H), 1.39-1.14 (m, 3H).

Step 2: tert-Butyl 10-((6-fluoro-4-oxoquinazolin-3(4H)-yl)methyl)-7-azaspiro[4.5]decane-7-carboxylate: Prepared according General Procedure 4 using 6-fluoroquinazolin-4(3H)-one (0.45 g, 2.6 mmol), tert-butyl 10-(iodomethyl)-7-azaspiro[4.5]decane-7-carboxylate (1.09 g, 2.9 mmol) and cesium carbonate (1.70 g, 5.2 mmol) to give the title compound (0.070 g, 5%). LCMS (Method F): R_{T}=4.13 min, m/z 360 [M−butene+H]⁺.

Step 3: 3-((7-Azaspiro[4.5]decan-10-yl)methyl)-6-fluoroquinazolin-4(3H)-one: Prepared according to General Procedure 1 using tert-butyl 10-((6-fluoro-4-oxoquinazolin-3(4H)-yl)methyl)-7-azaspiro[4.5]decane-7-carboxylate (21.7 mg, 0.052 mmol) and TFA (0.2 mL), stirring for 20 min to give the title compound (11.8 mg, 72%). LCMS (Method B): R_{T}=0.61 min, m/z 316 [M+H]⁺.

Step 4: tert-Butyl ((2S,4R)-1-(10-((6-fluoro-4-oxoquinazolin-3(4H)-yl)methyl)-7-azaspiro[4.5]decane-7-carbonyl)-2-phenylpiperidin-4-yl)carbamate: Prepared according to General Procedure 2 using triphosgene (5.6 mg, 0.019 mmol), pyridine (15.1 μL, 0.19 mmol), tert-butyl ((2S,4R)-2-phenylpiperidin-4-yl)carbamate (12.4 mg, 0.045 mmol), 3-((7-azaspiro[4.5]decan-10-yl)methyl)-6-fluoroquinazolin-4(3H)-one (11.8 mg, 0.037 mmol) and DIPEA (32.7 μL, 0.19 mmol) to give the title compound (21.7 mg, 94%). LCMS (Method A): R_{T}=1.76 min. m/z 618 [M+H]⁺.

Step 5: 3-((7-((2S,4R)-4-Amino-2-phenylpiperidine-1-carbonyl)-7-azaspiro[4.5]decan-10-yl)methyl)-6-fluoroquinazolin-4(3H)-one: Prepared according to General Procedure 1 using tert-butyl ((2S,4R)-1-(10-((6-fluoro-4-oxoquinazolin-3(4H)-yl)methyl)-7-azaspiro[4.5]decane-7-carbonyl)-2-phenylpiperidin-4-yl)carbamate (21.7 mg, 0.035 mmol) and TFA (0.2 mL), stirring for 20 min to give the title compound (15 mg, 75%). LCMS (Method B): R_{T}=0.83 min, m/z 518 [M+H]⁺. ¹H NMR (500 MHz, DMSO-d₆): δ 8.40 (s, 0.5H), 8.36 (s, 0.5H), 7.86-7.80 (m, 1H), 7.80-7.68 (m, 2H), 7.27 (t, 1H), 7.24-7.10 (m, 4H), 4.19-4.10 (m, 1H), 4.01-3.82 (m, 2H), 3.77 (t, 1H), 3.53 (d, 0.5H), 3.45 (d, 0.5H), 3.22 (dt, 0.5H), 3.14 (dt, 0.5H), 2.94 (t, 0.5H), 2.88-2.79 (m, 0.5H), 2.79-2.71 (m, 1H), 2.69-2.56 (m, 2H), 1.99-1.73 (m, 4.5H), 1.72-1.45 (m, 4H), 1.45-1.10 (m, 8H), 1.06-0.98 (m, 0.5H).

The Examples listed in the following table were prepared similarly to the procedure for Example 27 from commercially available nucleophiles or the intermediates described below to give the corresponding final compounds (Examples 28-43).

### Intermediate for Example 36: 6-(1-Methylcyclopropyl)pyrimidin-4(3H)-one

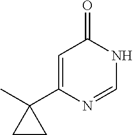

To methyl 3-(1-methylcyclopropyl)-3-oxopropanoate (3.00 g, 19 mmol) in methanol (80 mL) was added formamidine acetate (4.00 g, 38 mmol) and sodium methoxide (7.30 g, 134 mmol). The reaction mixture was heated under reflux for 2 days. The solvents were evaporated and remaining residue was diluted with water and made acidic with HCl (aq) solution. The precipitate was filtered, washed with water and MTBE, and dried under vacuum to give the title compound (1.50 g, 52%). LCMS (Method F): R_{T}=0.79 min, m/z 151 [M+H]⁺. ¹H NMR (500 MHz, DMSO-d₆): δ 12.21 (s, 1H), 8.01 (s, 1H), 6.18 (s, 1H), 1.31 (s, 3H), 1.17 (d, 2H), 0.75 (d, 2H).

### Intermediate for Example 41: tert-Butyl 10-((3-oxomorpholino)methyl)-7-azaspiro[4.5]decane-7-carboxylate

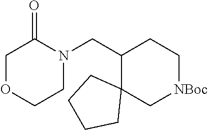 [SMILES: CC(C)(C)OC(=O)N1CCC(CN2CCOCC2=O)C2(CCCC2)C1]

Step 1: tert-Butyl 10-formyl-7-azaspiro[4.5]decane-7-carboxylate: To tert-butyl 10-(hydroxymethyl)-7-azaspiro[4.5]decane-7-carboxylate (8.00 g, 29.8 mmol) in DCM (150 mL) was added 1,1,1-tris(acetyloxy)-1,1-dihydro-1,2-benziodoxol-3-(1H)-one (Dess-Martin periodinane) (15.2 g, 35.8 mmol). After 3 h, the reaction mixture was treated with a solution of sodium thiosulfate (1 M in saturated potassium carbonate (aq) solution). The organic phase was decanted, dried (Na₂SO₄), and evaporated to dryness to yield the crude title compound that was used in the next step without further purification.

Step 2: tert-Butyl 10-(((2-((trimethylsilyl)oxy)ethyl)amino)methyl)-7-azaspiro[4.5]decane-7-carboxylate: To tert-butyl 10-formyl-7-azaspiro[4.5]decane-7-carboxylate (1.50 g, 5.6 mmol) in dichloroethane (40 mL) was added 2-((trimethylsilyl)oxy)ethanamine (0.75 g, 5.6 mmol) followed by sodium triacetoxyborohydride (3.58 g, 16.9 mmol). After 24 h, the solution was treated with saturated sodium hydrogencarbonate (aq) solution, the organic phase was decanted, dried (Na₂SO₄), and evaporated to dryness to yield the crude title compound (2.16 g, crude) that was used in the next step without further purification.

Step 3: tert-Butyl 10-((3-oxomorpholino)methyl)-7-azaspiro[4.5]decane-7-carboxylate: To tert-butyl 10-(((2-((trimethylsilyl)oxy)ethyl)amino)methyl)-7-azaspiro[4.5]decane-7-carboxylate (2.16 g, crude) in DCM (50 mL) at 0° C. were added 10% w/v sodium hydroxide (aq) solution (50 mL) and chloroacetyl chloride (0.63 g, 5.6 mmol) followed by trimethylbenzylammonium chloride (0.1 g). After 10 h, the organic phase was separated, dried (Na₂SO₄), and evaporated to dryness and purified by preparative HPLC to yield the title compound (0.32 g, 16%). LCMS (Method F): R_{T}=3.50 min, m/z 297 [M−butene+H]⁺. ¹H NMR (500 MHz, DMSO-d₆): δ 4.02 (s, 2H), 3.80 (m, 2H), 3.71 (m, 1H), 3.60 (m, 1H), 3.45-3.20 (m, 3H), 3.05 (m, 1H), 2.95-2.60 (m, 2H), 1.79 (m, 2H), 1.65 (m, 2H), 1.53 (m, 2H), 1.38 (s, 9H), 1.40-1.14 (m, 5H).

### Intermediate for Example 42: tert-Butyl 10-((6-oxo-5,6-dihydroimidazo[1,2-a]pyrazin-7(8H)-yl)methyl)-7-azaspiro[4.5]decane-7-carboxylate

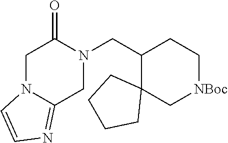 [SMILES: CC(C)(C)OC(=O)N1CCC(CN2Cc3nccn3CC2=O)C2(CCCC2)C1]

Step 1: tert-Butyl 10-((((1H-imidazol-2-yl)methyl)amino)methyl)-7-azaspiro[4.5]decane-7-carboxylate: To tert-butyl 10-formyl-7-azaspiro[4.5]decane-7-carboxylate (1.50 g, 5.6 mmol) in dichloroethane (40 mL) were added 2-aminomethylimidazole dihydrochloride (0.96 g, 5.6 mmol) and sodium acetate (1.39 g, 16.9 mmol) followed by sodium triacetoxyborohydride (3.58 g, 16.9 mmol). After 24 h, the solution was treated with saturated sodium hydrogencarbonate (aq) solution, the organic phase was decanted, dried (Na₂SO₄), and evaporated to dryness to yield the title compound (1.90 g, crude) that was used in next step without further purification.

Step 2: tert-Butyl 10-((6-oxo-5,6-dihydroimidazo[1,2-a]pyrazin-7(8H)-yl)methyl)-7-azaspiro[4.5]decane-7-carboxylate: To tert-butyl 10-((((1H-imidazol-2-yl)methyl)amino)methyl)-7-azaspiro[4.5]decane-7-carboxylate (1.90 g, crude) in DCM (50 mL) at 0° C. were added a 10% w/v sodium hydroxide (aq) solution (50 mL) and chloroacetyl chloride (0.63 g, 5.6 mmol) followed by trimethylbenzylammonium chloride (0.1 g). After 10 h, the organic phase was separated, dried (Na₂SO₄), evaporated to dryness and purified by preparative HPLC to give the title compound (0.11 g, 8%). LCMS (Method F): R_{T}=2.52 min, m/z 389 [M+H]⁺. H NMR (500 MHz, DMSO-d₆): (7.09 (s, 1H), 6.95 (s, 1H), 4.69 (s, 2H), 4.62-4.45 (m, 2H), 3.71 (m, 2H), 3.49 (m, 1H), 3.20 (m, 1H), 2.95-2.60 (m, 2H), 1.79 (m, 2H), 1.65 (m, 2H), 1.53 (m, 2H), 1.38 (s, 9H), 1.40-1.14 (m, 5H).

| | | | ¹H NMR | LCMS |
|---|---|---|---|---|
| | | | (500 MHz, DMSO-d₆): | (Method): |
| Example | Structure | Name | δ | R_{T}, m/z |
| 28 | 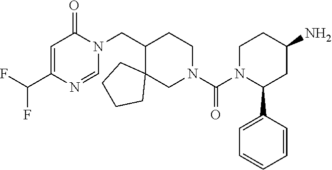 [SMILES: N[C@@H]1CCN(C(=O)N2CCC(Cn3cnc(C(F)F)cc3=O)C3(CCCC3)C2)[C@H](c2ccccc2)C1] | 3-((7-((2S,4R)-4- Amino- 2-phenylpiperidine- 1- carbonyl)-7- azaspiro[4.5]decan- 10-yl)methyl)-6- (difluoromethyl) pyrimidin- 4(3H)-one | 8.62 (s, 0.5H), 8.58 (s, 0.5H), 7.29-7.11 (m, 5H), 6.74 (dt, 1H), 6.66 (d, 1H), 4.12- 4.02 (m, 1H), 4.00-3.89 (m, 1.5H), 3.86 (dt, 0.5H), 3.76-3.65 (m, 1H), 3.51 (d, 0.5H), 3.43 (d, 0.5H), 3.22 (dt, 0.5H), 3.16 (dt, 0.5H), 2.95 (t, 0.5H), 2.88-2.75 (m, 1.5H), 2.70-2.64 (m, 1H), 1.90-1.70 (m, 4H), 1.69-1.10 (m, 11.5H), 1.04-0.95 (m, 0.5H). | (Method B) RT = 0.71 min, m/z 500 [M + H]⁺ |
| 29 | 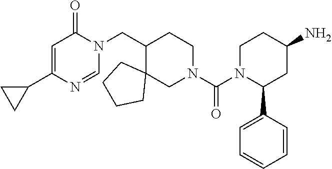 [SMILES: N[C@@H]1CCN(C(=O)N2CCC(Cn3cnc(C4CC4)cc3=O)C3(CCCC3)C2)[C@H](c2ccccc2)C1] | 3-((7-((2S,4R)-4- Amino- 2-phenylpiperidine- 1-carbonyl)-7- azaspiro[4.5]decan- 10-yl)methyl)-6- cyclopropyl- pyrimidin- 4(3H)-one | 8.31 (s, 0.5H), 8.27 (s, 0.5H), 7.27-7.10 (m, 5H), 6.30 (d, 1H), 4.04-3.95 (m, 1H), 3.95-3.88 (m, 1.5H), 3.85 (dt, 0.5H), 3.63- 3.54 (m, 1H), 3.49 (d, 0.5H), 3.41 (d, 0.5H), 3.20 (dt, 0.5H), 3.15 (dt, 0.5H), 2.94 (t, 0.5H), 2.87-2.70 (m, 1.5H), 2.63- 2.58 (m, 1H), 1.90-1.69 (m, 5H), 1.67- 1.09 (11.5H), 1.02-0.95 (m, 0.5H), 0.91 (t, 4H). | (Method B) RT = 0.75 min, m/z 490 [M +H ]⁺ |
| 30 | 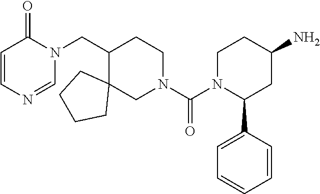 [SMILES: N[C@@H]1CCN(C(=O)N2CCC(Cn3cnccc3=O)C3(CCCC3)C2)[C@H](c2ccccc2)C1] | 3-((7-((2S,4R)-4- Amino- 2-phenylpiperidine- 1-carbonyl)-7- azaspiro[4.5]decan- 10- yl)methyl) pyrimidin- 4(3H)-one | 8.47 (s, 0.5H), 8.43 (s, 0.5H), 7.96 (t, 1H), 7.31-7.08 (m, 5H), 6.42-6.36 (m, 1H), 4.10-4.01 (m, 1H), 4.00-3.82 (m, 2H), 3.71-3.60 (m, 1H), 3.50 (d, 0.5H), 3.42 (0.5H), 3.21 (dt, 0.5H), 3.15 (dt, 0.5H), 2.95 (t, 0.5H), 2.90-2.80 (m, 0.5H), 2.80- 2.71 (m, 1H), 2.69-2.58 (m, 2H), 1.87- 1.70 (m, 4H), 1.69-1.51 (m, 2H), 1.50- 1.41 (m, 1.5H), 1.39-1.10 (m, 7H), 1.03- 0.95 (m, 0.5H). | (Method B) RT = 0.64 min, m/z 450 [M + H]⁺ |
| 31 | 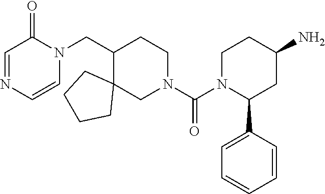 [SMILES: N[C@@H]1CCN(C(=O)N2CCC(Cn3ccncc3=O)C3(CCCC3)C2)[C@H](c2ccccc2)C1] | 1-((7-((2S,4R)-4- Amino- 2-phenylpiperidine- 1-carbonyl)-7- azaspiro[4.5]decan- 10- yl)methyl)pyrazin- 2(1H)-one | 8.01-7.97 (m, 1H), 7.66 (d, 0.5H), 7.63 (d, 0.5H), 7.33 (d, 0.5H), 7.31 (d, 0.5H), 7.29-7.12 (m, 5H), 4.28 (br s, 2H), 4.06- 3.98 (m, 1H), 3.98-3.89 (m, 1.5H), 3.83 (dt, 0.5H), 3.73-3.63 (m, 1H), 3.49 (d, 0.5H), 3.39 (d, 0.5H), 3.22 (dt, 0.5H), 3.17 (dt, 0.5H), 2.97 (t, 0.5H), 2.92-2.84 (m, 1.5H), 2.71-2.61 (m, 1H), 1.92-1.78 (m, 3H), 1.70-1.08 (m, 12.5H), 1.02-0.95 (m, 0.5H). | (Method B) RT = 0.61 min, m/z 450 [M + H]⁺ |
| 32 | 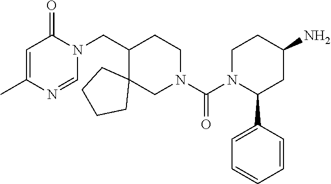 [SMILES: Cc1cc(=O)n(CC2CCN(C(=O)N3CC[C@@H](N)C[C@H]3c3ccccc3)CC23CCCC3)cn1] | 3-((7-((2S,4R)-4- Amino- 2-phenylpiperidine- 1-carbonyl)-7- azaspiro[4.5]decan- 10- yl)methyl)-6- methylpyrimidin- 4(3H)-one | 8.37 (s, 0.5H), 8.33 (s, 0.5H), 7.29-7.12 (m, 5H), 6.25 (s, 0.5H), 6.24 (s, 0.5H), 4.06-3.97 (m, 1H), 3.97-3.89 (m, 1.5H), 3.85 (dt, 0.5H), 3.69-3.58 (m, 1H), 3.49 (d, 0.5H), 3.41 (d, 0.5H), 3.22 (dt, 0.5H), 3.17 (dt, 0.5H), 2.95 (t, 0.5H), 2.90-2.76 (m, 1.5H), 2.71-2.60 (m, 1H), 2.19 (s, 1.5H), 2.18 (s, 1.5H), 1.91-1.69 (m, 4H), 1.69-1.09 (11.5H), 1.06-0.93 (m, 0.5H). | (Method B) RT = 0.63 min, m/z 464 [M + H]⁺ |
| 33 | 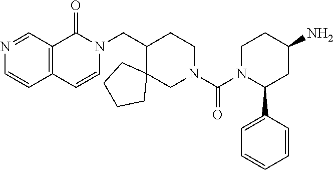 [SMILES: N[C@@H]1CCN(C(=O)N2CCC(Cn3ccc4ccncc4c3=O)C3(CCCC3)C2)[C@H](c2ccccc2)C1] | 2-((7-((2S,4R)-4- Amino- 2-phenylpiperidine- 1-carbonyl)-7- azaspiro[4.5]decan- 10-yl)methyl)-2,7- naphthyridin- 1(2H)-one | 9.35 (s, 0.5H), 9.34 (s, 0.5H), 8.74-8.66 (m, 1H), 7.74 (d, 0.5H), 7.71 (d, 0.5H), 7.61-7.56 (m, 1H), 7.29 (t, 1H), 7.24-7.11 (m, 4H), 6.67-6.61 (m, 1H), 4.18-4.08 (m, 1H), 3.98-3.89 (m, 1.5H), 3.85 (dt, 0.5H), 3.82-3.72 (m, 1H), 3.51 (d, 0.5H), 3.42 (d, 0.5H), 3.22 (dt, 0.5H, 3.15 (dt, 0.5H), 2.96 (t, 0.5H), 2.90-2.74 (m, 0.5H), 2.82-2.74 (m, 1H), 2.71-2.64 (m, 1H), 1.92-1.74 (m, 4H), 1.72-1.43 (m, 4H), 1.43-1.14 (m, 7.5H), 1.06-0.98 (m, 0.5H). | (Method B) RT = 0.66 min, m/z 500 [M + H]⁺ |
| 34 | 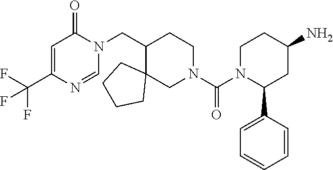 [SMILES: N[C@@H]1CCN(C(=O)N2CCC(Cn3cnc(C(F)(F)F)cc3=O)C3(CCCC3)C2)[C@H](c2ccccc2)C1] | 3-((7-((2S,4R)-4- Amino-2- phenylpiperidine- 1-carbonyl)-7- azaspiro[4.5]decan- 10-yl)methyl)-6- (trifluoromethyl) pyrimidin- 4(3H)-one | 8.70 (s, 0.5H), 8.66 (s, 0.5H), 7.30-7.13 (m, 5H), 6.96 (s, 0.5H), 6.95 (s, 0.5H), 5.25 (br s, 2H), 4.13-4.03 (m, 1H), 4.01- 3.90 (m, 1.5H), 3.95 (dt, 0.5H), 3.78-3.67 (m, 1H), 3.52 (d, 0.5H), 3.43 (d, 0.5H), 3.73 (dt, 0.5H), 3.19 (dt, 0.5H), 3.03-2.90 (m, 1.5H), 2.89-2.77 (m, 0.5H), 2.71-2.65 (m, 1H), 1.94-1.79 (m, 3H), 1.79-1.70 (m, 1H), 1.69-1.10 (m, 11.5H), 1.01-0.93 (m, 0.5H). | (Method B) RT = 0.82 min, m/z 518 [M + H]⁺ |
| 35 | 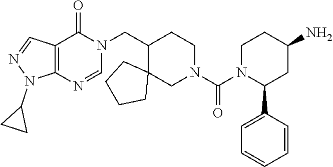 [SMILES: N[C@@H]1CCN(C(=O)N2CCC(Cn3cnc4c(cnn4C4CC4)c3=O)C3(CCCC3)C2)[C@H](c2ccccc2)C1] | 5-((7-((2S,4R)-4- Amino- 2-phenylpiperidine- 1-carbonyl)-7- azaspiro[4.5]decan- 10-yl)methyl)-1- cyclopropyl- 1,5-dihydro-4H- pyrazolo[3,4-d] pyrimidin- 4-one | 8.41 (s, 0.5H), 8.37 (s, 0.5H), 8.01 (s, 0.5H), 8.00 (s, 0.5H), 7.27 (t, 1H), 7.25- 7.12 (m, 4H), 4.52 (br s , 2H), 4.15-4.07 (m, 1H), 3.99-3.90 (m, 1.5H), 3.88-3.81 (m, 1.5H), 3.81-3.73 (m, 1H), 3.51 (d, 0.5H), 3.44 (d, 0.5H), 3.23 (dt, 0.5H), 3.17 (dt, 0.5H), 2.98-2.86 (m, 1.5H), 2.86-2.73 (m, 0.5H), 2.70-2.64 (m, 1H), 1.92-1.73 (m, 4H), 1.72-1.11 (m, 13.5H), 1.11-1.05 (m, 2H), 1.03-0.95 (m, 0.5H). | (Method B) RT = 0.79 min, m/z 530 [M + H]⁺ |
| 36 | 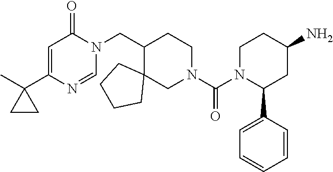 [SMILES: CC1(c2cc(=O)n(CC3CCN(C(=O)N4CC[C@@H](N)C[C@H]4c4ccccc4)CC34CCCC4)cn2)CC1] | 3-((7-((2S,4R)-4- Amino- 2-phenylpiperidine- 1-carbonyl)-7- azaspiro[4.5]decan- 10-yl)methyl)-6-(1- methylcyclopropyl) pyrimidin- 4(3H)-one | 8.30 (s, 0.5H), 8.26 (s, 0.5H), 7.29-7.11 (m, 5H), 6.26 (s, 0.5H), 6.25 (s, 0.5H), 4.04-3.97 (m, 1H), 3.97-3.89 (m, 1.5H), 3.85 (dt, 0.5H), 3.64-3.55 (m, 1H), 3.49 (d, 0.5H), 3.42 (d, 0.5H), 3.21 (dt, 0.5H), 3.15 (dt, 0.5H), 2.98-2.91 (t, 0.5H), 2.87- 2.75 (m, 1.5H), 2.68-2.59 (m, 1H), 1.88- 1.69 (m, 4H), 1.68-1.05 (m, 16.5H), 1.02- 0.95 (m, 0.5H), 0.82-0.75 (m, 2H). | (Method B) RT = 0.85 min, m/z 504 [M + H]⁺ |
| 37 | 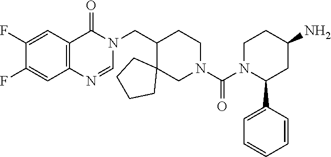 [SMILES: N[C@@H]1CCN(C(=O)N2CCC(Cn3cnc4cc(F)c(F)cc4c3=O)C3(CCCC3)C2)[C@H](c2ccccc2)C1] | 3-((7-((2S,4R)-4- Amino-2- phenylpiperidine- 1-carbonyl)-7- azaspiro[4.5]decan- 10-yl)methyl)-6,7- difluoroquinazolin- 4(3H)-one | 8.32 (s, 0.5H), 8.30 (s, 0.5H), 8.11-8.04 (m, 1H), 7.64-7.57 (m, 1H), 7.34-7.16 (m, 5H), 4.30-4.19 (m, 1.5H), 4.07-3.98 (m, 1.5H), 3.90 (t, 0.5H), 3.84 (t, 0.5H), 3.70 (d, 0.5H), 3.54 (d, 0.5H), 3.41 (dt, 0.5H), 3.10-3.01 (m, 0.5H), 3.01-2.90 (m, 1H), 2.88-2.75 (m, 1.5H), 2.58 (d, 0.5H), 2.04- 1.94 (m, 3H), 1.90-1.82 (m, 0.5H), 1.81- 1.67 (m, 2.5H), 1.63-1.42 (m, 7H), 1.15 (d, 0.5H), 1.05-0.99 (m, 0.5H), 0.97-0.84 (m, 2H). | (Method B) RT = 0.92 min, m/z 536 [M + H]⁺ |
| 38 | 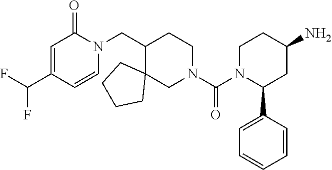 [SMILES: N[C@@H]1CCN(C(=O)N2CCC(Cn3ccc(C(F)F)cc3=O)C3(CCCC3)C2)[C@H](c2ccccc2)C1] | 1-((7-((2S,4R)-4- Amino- 2-phenylpiperidine- 1-carbonyl)-7- azaspiro[4.5]decan- 10-yl)methyl)-4- (difluoromethyl) pyridin- 2(1H)-one | 7.84 (d, 0.5H), 7.81 (d, 0.5H), 7.29-7.09 (m, 5H), 6.87 (t, 0.5H), 6.86 (t, 0.5H), 6.61-6.54 (m, 1H), 6.36-6.29 (m, 1H), 4.13-4.03 (m, 1H), 3.99-3.81 (m, 2H), 3.75-3.64 (m, 1H), 3.49 (d, 0.5H), 3.40 (d, 0.5H), 3.20 (dt, 0.5H), 3.14 (dt, 0.5H), 2.96 (t, 0.5H), 2.91-2.78 (m, 0.5H), 2.74- 2.55 (m, 3H), 2.06-1.52 (m, 7.5H), 1.52- 1.39 (m, 2H), 1.38-1.09 (m, 7H), 1.06- 0.95 (m, 0.5H). | (Method B) RT = 0.81 min, m/z 499 [M + H]⁺ |
| 39 | 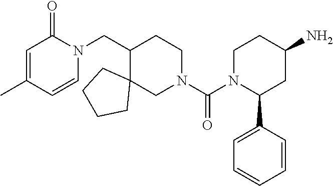 [SMILES: Cc1ccn(CC2CCN(C(=O)N3CC[C@@H](N)C[C@H]3c3ccccc3)CC23CCCC3)c(=O)c1] | 1-((7-((2S,4R)-4- Amino- 2-phenylpiperidine- 1-carbonyl)-7- azaspiro[4.5]decan- 10-yl)methyl)-4- methylpyridin- 2(1H)-one | 7.56-7.47 (m, 1H), 7.29-7.11 (m, 5H), 6.21-6.145 (m, 1H), 6.09-6.03 (m, 1H), 4.02-3.89 (m, 2.5H), 3.83 (dt, 0.5H), 3.67- 3.56 (m, 1H), 3.47 (d, 0.5H), 3.38 (d, 0.5H), 3.22 (dt, 0.5H), 3.17 (dt, 0.5H), 2.95 (t, 0.5H), 2.91-2.80 (m, 1H), 2.71- 2.56 (m, 1.5H), 2.15-2.07 (m, 3H), 1.93- 1.69 (m, 4H), 1.68-1.07 (m, 11.5H), 1.03- 0.94 (m, 0.5H). | (Method B) RT = 0.78 min, m/z 463 [M + H]⁺ |
| 40 | 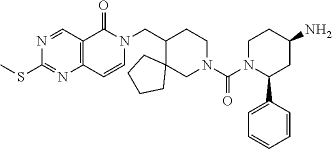 [SMILES: CSc1ncc2c(=O)n(CC3CCN(C(=O)N4CC[C@@H](N)C[C@H]4c4ccccc4)CC34CCCC4)ccc2n1] | 6-((7-((2S,4R)-4- Amino- 2-phenylpiperidine- 1-carbonyl)-7- azaspiro[4.5]decan- 10-yl)methyl)-2- (methylthio)pyrido [4,3-d]pyrimidin- 5(6H)-one | 9.24-9.20 (m, 1H), 7.99 (d, 0.5H), 7.95 (d, 0.5H), 7.29-7.10 (m, 5H), 6.56-6.49 (m, 1H), 4.15-4.06 (m, 1H), 3.98-3.88 (m, 1.5H), 3.85 (dt, 0.5H), 3.80-3.72 (m, 1H), 3.50 (d, 0.5H), 3.41 (d, 0.5H), 3.21(dt, 0.5H), 3.14 (dt, 0.5H), 2.95 (t, 0.5H), 2.91- 2.80 (m, 0.5H), 2.76-2.69 (m, 1H), 2.68- 2.61 (m, 1H), 2.61-2.57 (m, 3H), 1.89- 1.73 (m, 4H), 1.70-1.42 (m, 4H), 1.42- 1.14 (m, 7.5H), 1.06-0.97 (m, 0.5H). | (Method B) RT = 0.93 min, m/z 547 [M + H]⁺ |
| 41 | 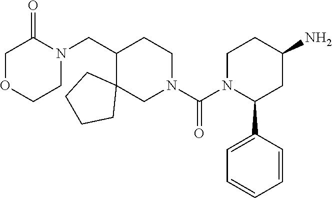 [SMILES: N[C@@H]1CCN(C(=O)N2CCC(CN3CCOCC3=O)C3(CCCC3)C2)[C@H](c2ccccc2)C1] | 4-((7-((2S,4R)-4- Amino-2- phenylpiperidine-1- carbonyl)-7- azaspiro[4.5]decan- 10-yl)methyl) morpholin-3-one | 7.25-7.10 (m, 5H), 4.07-3.98 (m, 2H), 3.94-3.76 (m, 4H), 3.67-3.55 (m, 1H), 3.45-3.34 (m, 2H), 3.21-3.13 (m, 1H), 3.11-2.98 (m, 2H), 2.73-2.53 (m, 3.5H), 1.84-0.94 (m, 17.5H). | (Method B) 0.70 min, 455 [M + H]⁺ |
| 42 | 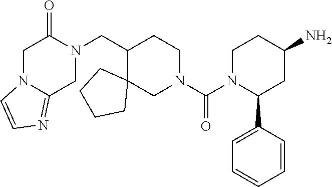 [SMILES: N[C@@H]1CCN(C(=O)N2CCC(CN3Cc4nccn4CC3=O)C3(CCCC3)C2)[C@H](c2ccccc2)C1] | 7-((7-((2S,4R)-4- Amino- 2-phenylpiperidine- 1-carbonyl)-7- azaspiro[4.5]decan- 10-yl)methyl)-7,8- dihydroimidazo[1,2- a]pyrazin- 6(5H)-one | 7.26-7.08 (m, 6H), 6.97-6.94 (m, 1H), 4.74-4.64 (m, 2H), 4.63-4.49 (m, 2H), 3.95-3.66 (m, 3.5H), 3.47-3.40 (m, 0.5H), 3.22-3.12 (m, 2H), 3.09-2.92 (m, 1H), 2.75-2.54 (m, 3.5H), 1.88-1.73 (m, 3H), 1.67-1.08 (m, 13H), 1.04-0.95 (m, 0.5H). | (Method B) 0.47 min, 491 [M + H]⁺ |
| 43 | 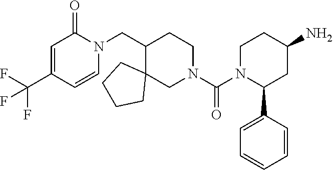 [SMILES: N[C@@H]1CCN(C(=O)N2CCC(Cn3ccc(C(F)(F)F)cc3=O)C3(CCCC3)C2)[C@H](c2ccccc2)C1] | 1-((7-((2S,4R)-4- Amino- 2-phenylpiperidine- 1-carbonyl)-7- azaspiro[4.5]decan- 10-yl)methyl)-4- (trifluoromethyl) pyridin- 2(1H)-one | 8.00-7.91 (m, 1H), 7.28-7.09 (m, 5H), 6.79-6.73 (m, 1H), 6.51-6.44 (m, 1H), 4.14-4.06 (m, 1H), 3.99-3.81 (m, 2H), 3.78-3.68 (m, 1H), 3.54-3.38 (m, 1H), 3.23-3.11 (m, 1H), 3.00-2.90 (m, 0.5H), 2.89-2.77 (m, 0.5H), 2.73-2.56 (m, 2.5H), 1.88-1.10 (m, 16H), 1.04-0.95 (m, 0.5H). | (Method B) 0.90 min, 517 [M + H]⁺ |

### Example 44: 1-(((R)-7-((2S,4R)-4-Hydroxy-2-phenylpiperidine-1-carbonyl)-7-azaspiro[4.5]decan-10-yl)methyl)-4-phenylpyridin-2(1H)-one

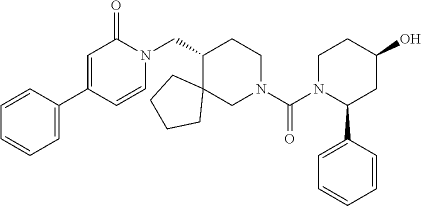 [SMILES: O=C(N1CC[C@@H](Cn2ccc(-c3ccccc3)cc2=O)C2(CCCC2)C1)N1CC[C@@H](O)C[C@H]1c1ccccc1]

Step 1: tert-Butyl 10-((4-chloro-2-oxopyridin-1(2H)-yl)methyl)-7-azaspiro[4.5]decane-7-carboxylate: Prepared according General Procedure 4 using 4-chloropyridone (5.00 g, 38.6 mmol), tert-butyl 10-(bromomethyl)-7-azaspiro[4.5]decane-7-carboxylate (14.1 g, 42.5 mmol) and cesium carbonate (25.2 g, 77.2 mmol) to give the title compound (4.00 g, 27%). LCMS (Method F): R_{T}=1.52 min, m/z=281 [M−Boc+H]⁺. ¹H NMR (CDCl₃): δ 7.11 (d, 1H), 6.59 (d, 1H), 6.17 (m, 1H), 4.31 (m, 1H), 4.02 (m, 1H), 3.75-3.40 (m, 2H), 2.79 (m, 1H), 2.63 (d, 1H), 1.95 (m, 2H), 1.80-1.25 (d, 1H), 1.43 (s, 9H).

Step 2: tert-Butyl (R)-10-((4-chloro-2-oxopyridin-1(2H)-yl)methyl)-7-azaspiro[4.5]decane-7-carboxylate and tert-butyl (S)-10-((4-chloro-2-oxopyridin-1(2H)-yl)methyl)-7-azaspiro[4.5]decane-7-carboxylate: tert-Butyl 10-((4-chloro-2-oxopyridin-1(2H)-yl)methyl)-7-azaspiro[4.5]decane-7-carboxylate (4.00 g) was resolved into the single stereoisomers by chiral HPLC using a Chiralpak IA (20 mm×250 mm, 5 μm) column with isocratic solvent conditions: 70:15:15 hexane/IPA/MeOH. The first eluted material afforded tert-butyl (R)-10-((4-chloro-2-oxopyridin-1(2H)-yl)methyl)-7-azaspiro[4.5]decane-7-carboxylate (1.54 g). [α]_{D}²⁵=+84.2 (c 0.25 in MeOH). The second eluted material afforded tert-butyl (S)-10-((4-chloro-2-oxopyridin-1(2H)-yl)methyl)-7-azaspiro[4.5]decane-7-carboxylate (1.52 g). [α]_{D}²⁵=−86.2 (c 0.25 in MeOH).

Step 3: tert-Butyl (R)-10-((2-oxo-4-phenylpyridin-1(2H)-yl)methyl)-7-azaspiro[4.5]decane-7-carboxylate: Prepared according to General Procedure 5 using tert-butyl (R)-10-((4-chloro-2-oxopyridin-1(2H)-yl)methyl)-7-azaspiro[4.5]decane-7-carboxylate (100 mg, 0.26 mmol), phenylboronic acid (64 mg, 0.53 mmol), Na₂CO₃ (84 mg, 0.79 mmol) and PdCl₂(dppf)·DCM (11.1 mg, 0.013 mmol) in 1,4-dioxane (1.2 mL) and water (0.4 mL) to give the title compound (106 mg, 95%). LCMS (Method B): R_{T}=1.56 min, m/z=367 [M−butene+H]⁺.

Step 4: (R)-1-((7-Azaspiro[4.5]decan-10-yl)methyl)-4-phenylpyridin-2(1H)-one: Prepared according to General Procedure 1 using tert-butyl (R)-10-((2-oxo-4-phenylpyridin-1(2H)-yl)methyl)-7-azaspiro[4.5]decane-7-carboxylate (110 mg, 0.26 mmol) and TFA (0.5 mL) in DCM (2 mL) to give the title compound (80 mg, 95%). LCMS (Method B): R_{T}=0.73 min, m/z=323 [M+H]⁺.

Step 5: 1-(((R)-7-((2S,4R)-4-((tert-Butyldimethylsilyl)oxy)-2-phenylpiperidine-1-carbonyl)-7-azaspiro[4.5]decan-10-yl)methyl)-4-phenylpyridin-2(1H)-one: Prepared according to General Procedure 2 using (R)-1-((7-azaspiro[4.5]decan-10-yl)methyl)-4-phenylpyridin-2(1H)-one (30.0 mg, 0.093 mmol), (2S,4R)-4-((tert-butyldimethylsilyl)oxy)-2-phenylpiperidine (29.8 mg, 0.102 mmol), triphosgene (11 mg, 0.037 mmol), pyridine (30 μL, 0.37 mmol) and DIPEA (52 μL, 0.37 mmol) in MeCN (1 mL) to give the title compound (40 mg, 67%). LCMS (Method B): R_{T}=2.04 min, m/z=640 [M+H]⁺.

Step 6: 1-(((R)-7-((2S,4R)-4-Hydroxy-2-phenylpiperidine-1-carbonyl)-7-azaspiro[4.5]decan-10-yl)methyl)-4-phenylpyridin-2(1H)-one: To a stirring solution of 1-(((R)-7-((2S,4R)-4-((tert-butyldimethylsilyl)oxy)-2-phenylpiperidine-1-carbonyl)-7-azaspiro[4.5]decan-10-yl)methyl)-4-phenylpyridin-2(1H)-one (40 mg, 0.063 mmol) in MeOH (1 mL) was added p-toluenesulfonic acid monohydrate (2.4 mg, 0.013 mmol) and the resulting solution stirred at rt for 18 h. The reaction was quenched with saturated NaHCO_{3(aq)} and extracted with DCM (×3) using a phase separator. The combined organic phases were concentrated in vacuo and the crude material was purified by flash chromatography (10-100% EtOAc in cyclohexane, then 0-20% MeOH in EtOAc) to give the title compound (16 mg, 48%). LCMS (Method B): R_{T}=1.27 min, m/z=526 [M+H]⁺. ¹H NMR (500 MHz, DMSO-d₆): δ 7.76-7.67 (m, 3H), 7.48 (q, J=6.6 Hz, 3H), 7.24-7.16 (m, 4H), 7.16-7.10 (m, 1H), 6.65 (d, J=2.0 Hz, 1H), 6.57 (dd, J=7.1, 2.0 Hz, 1H), 4.70 (d, J=4.4 Hz, 1H), 4.08 (dd, J=12.8, 3.1 Hz, 1H), 3.99 (dd, J=11.4, 3.1 Hz, 1H), 3.85 (dd, J=11.7, 6.5 Hz, 1H), 3.72 (t, J=11.8 Hz, 1H), 3.59 (tq, J=9.4, 4.4 Hz, 1H), 3.40 (d, J=12.9 Hz, 1H), 3.22 (dt, J=12.4, 4.0 Hz, 1H), 2.87 (s, 1H), 2.68 (t, J=12.0 Hz, 2H), 1.93-1.80 (m, 3H), 1.76 (s, 1H), 1.71-1.12 (m, 11H).

### Example 45: 1-(((R)-7-((2S,4R)-4-(Ethylamino)-2-phenylpiperidine-1-carbonyl)-7-azaspiro[4.5]decan-10-yl)methyl)-4-phenylpyridin-2(1H)-one

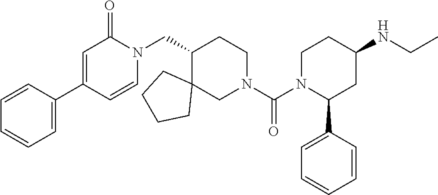 [SMILES: CCN[C@@H]1CCN(C(=O)N2CC[C@@H](Cn3ccc(-c4ccccc4)cc3=O)C3(CCCC3)C2)[C@H](c2ccccc2)C1]

Step 1: tert-Butyl ethyl((2S,4R)-1-((R)-10-((2-oxo-4-phenylpyridin-1(2H)-yl)methyl)-7-azaspiro[4.5]decane-7-carbonyl)-2-phenylpiperidin-4-yl)carbamate: Prepared according to General Procedure 2 using (R)-1-((7-azaspiro[4.5]decan-10-yl)methyl)-4-phenylpyridin-2(1H)-one (20.0 mg, 0.062 mmol), tert-butyl ethyl((2S,4R)-2-phenylpiperidin-4-yl)carbamate (20.6 mg, 0.074 mmol), triphosgene (6.4 mg, 0.022 mmol), pyridine (20 μL, 0.248 mmol) and DIPEA (43 μL, 0.248 mmol) in MeCN (1 mL) to give the title compound (32 mg, 79%). LCMS (Method B): R_{T}=1.76 min, m/z=653 [M+H]⁺.

Step 2: 1-(((R)-7-((2S,4R)-4-(Ethylamino)-2-phenylpiperidine-1-carbonyl)-7-azaspiro[4.5]decan-10-yl)methyl)-4-phenylpyridin-2(1H)-one: Prepared according to General Procedure 1 using tert-butyl ethyl((2S,4R)-1-((R)-10-((2-oxo-4-phenylpyridin-1(2H)-yl)methyl)-7-azaspiro[4.5]decane-7-carbonyl)-2-phenylpiperidin-4-yl)carbamate (30.0 mg, 0.046 mmol) and TFA (0.5 mL) in DCM (2 mL) to give the title compound (12 mg, 45%) after lyophilisation. LCMS (Method B): R_{T}=0.94 min, m/z=553 [M+H]⁺. ¹H NMR (500 MHz, DMSO-d₆): δ 7.75-7.67 (m, 3H), 7.51-7.43 (m, 3H), 7.25-7.16 (m, 4H), 7.16-7.09 (m, 1H), 6.65 (d, J=2.1 Hz, 1H), 6.57 (dd, J=7.1, 2.1 Hz, 1H), 4.08 (dd, J=12.8, 3.1 Hz, 1H), 3.96 (dd, J=11.7, 3.1 Hz, 1H), 3.91-3.81 (m, 1H), 3.71 (t, J=11.8 Hz, 1H), 3.41 (d, J=12.9 Hz, 1H), 3.27-3.21 (m, 1H), 2.86 (s, 1H), 2.72-2.64 (m, 2H), 2.60-2.54 (m, 2H, overlapping with solvent signal), 1.96-1.71 (m, 4H), 1.70-1.12 (m, 12H), 0.96 (t, J=7.1 Hz, 3H). [NH signal not observed].

### Example 46: 1-(((R)-7-((2S,4R)-4-(Dimethylamino)-2-phenylpiperidine-1-carbonyl)-7-azaspiro[4.5]decan-10-yl)methyl)-4-phenylpyridin-2(1H)-one

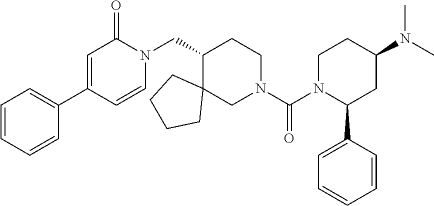 [SMILES: CN(C)[C@@H]1CCN(C(=O)N2CC[C@@H](Cn3ccc(-c4ccccc4)cc3=O)C3(CCCC3)C2)[C@H](c2ccccc2)C1]

To a solution of 1-(((R)-7-((2S,4R)-4-amino-2-phenylpiperidine-1-carbonyl)-7-azaspiro[4.5]decan-10-yl)methyl)-4-phenylpyridin-2(1H)-one (30.0 mg, 0.057 mmol) in MeCN (0.6 mL) and MeOH (0.6 mL) was added formaldehyde (37% aqueous solution, 85 μL, 1.14 mmol) followed by sodium triacetoxyhydroborate (242 mg, 1.14 mmol) in 4 portions every 15 min. The reaction was stirred for a further 3 h. The reaction mixture was quenched with saturated NaHCO_{3(aq)} and extracted with DCM (×3) using a phase separator. The combined organic phase was concentrated in vacuo and the crude material was purified by flash chromatography using a Biotage Sfar Amino D column (0-100% DCM in cyclohexane; then 0-15% MeOH in DCM) and freeze-dried to give the title compound (23.7 mg, 74%). LCMS (Method B): R_{T}=0.94 min, m/z=553 [M+H]⁺. ¹H NMR (500 MHz, DMSO-d₆): δ 7.75-7.68 (m, 3H), 7.52-7.45 (m, 3H), 7.21 (h, J=6.0 Hz, 4H), 7.14 (td, J=6.3, 2.5 Hz, 1H), 6.66 (d, J=2.1 Hz, 1H), 6.57 (dd, J=7.1, 2.1 Hz, 1H), 4.08 (dd, J=12.8, 3.2 Hz, 1H), 3.94 (dd, J=11.8, 3.1 Hz, 1H), 3.87 (dd, J=13.3, 4.5 Hz, 1H), 3.72 (t, J=11.7 Hz, 1H), 3.40 (d, J=12.9 Hz, 1H), 2.89 (s, 1H), 2.72-2.64 (m, 2H), 2.31 (tt, J=11.7, 4.0 Hz, 1H), 2.15 (s, 6H), 1.89-1.71 (m, 4H), 1.69-1.42 (m, 5H), 1.41-1.13 (m, 6H). [Missing 1H signal obscured by HDO].

### Example 47: 1-((1-((2S,4R)-4-(Methylamino)-2-phenylpiperidine-1-carbonyl)piperidin-4-yl)methyl)-4-phenylpyridin-2(1H)-one

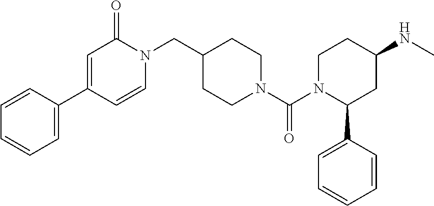 [SMILES: CN[C@@H]1CCN(C(=O)N2CCC(Cn3ccc(-c4ccccc4)cc3=O)CC2)[C@H](c2ccccc2)C1]

Step 1: 2,2,2-Trifluoro-N-methyl-N-((2S,4R)-1-(4-((2-oxo-4-phenylpyridin-1(2H)-yl)methyl)piperidine-1-carbonyl)-2-phenylpiperidin-4-yl)acetamide: Prepared according to General Procedure 2 using 4-phenyl-1-(piperidin-4-ylmethyl)pyridin-2(1H)-one (20.0 mg, 0.075 mmol), 2,2,2-trifluoro-N-methyl-N-((2S,4R)-2-phenylpiperidin-4-yl)acetamide hydrochloride (24.0 mg, 0.075 mmol), triphosgene (8.8 mg, 0.030 mmol), pyridine (30 μL, 0.373 mmol) and DIPEA (65 μL, 0.373 mmol) in MeCN (1 mL) to give the title compound (17 mg, 39%). LCMS (Method B): R_{T}=1.76 min, m/z=653 [M+H]⁺.

Step 2: 1-((1-((2S,4R)-4-(Methylamino)-2-phenylpiperidine-1-carbonyl)piperidin-4-yl)methyl)-4-phenylpyridin-2(1H)-one: Prepared according to General Procedure 6 using 2,2,2-trifluoro-N-methyl-N-((2S,4R)-1-(4-((2-oxo-4-phenylpyridin-1(2H)-yl)methyl)piperidine-1-carbonyl)-2-phenylpiperidin-4-yl)acetamide (17.0 mg, 0.029 mmol) and potassium carbonate (20.2 mg, 0.146 mmol) in MeOH (2 mL) and water (0.2 mL) to give the title compound (13 mg, 87%). LCMS (Method B): R_{T}=0.79 min, m/z=485 [M+H]⁺. ¹H NMR (500 MHz, DMSO-d₆): δ 7.76-7.68 (m, 3H), 7.52-7.43 (m, 3H), 7.23 (dd, J=8.1, 7.1 Hz, 2H), 7.21-7.15 (m, 2H), 7.14-7.08 (m, 1H), 6.67 (d, J=2.0 Hz, 1H), 6.57 (dd, J=7.1, 2.1 Hz, 1H), 3.96 (td, J=11.9, 9.7, 5.9 Hz, 3H), 3.85-3.74 (m, 2H), 3.36-3.22 (m, 2H, obscured by HDO signal), 2.80-2.66 (m, 2H), 2.48-2.41 (1H, m, obscured by DMSO signal), 2.25 (s, 3H), 2.03-1.86 (m, 3H), 1.57 (d, J=13.0 Hz, 1H), 1.50 (d, J=12.9 Hz, 1H), 1.38-1.28 (m, 1H), 1.26-0.94 (m, 3H). [NH signal not observed].

### Example 48: 1-(((R)-7-((2S,4R)-4-(Methylamino)-2-phenylpiperidine-1-carbonyl)-7-azaspiro[4.5]decan-10-yl)methyl)-4-phenylpyridin-2(1H)-one

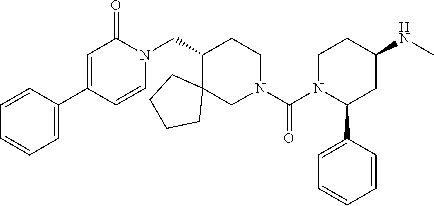 [SMILES: CN[C@@H]1CCN(C(=O)N2CC[C@@H](Cn3ccc(-c4ccccc4)cc3=O)C3(CCCC3)C2)[C@H](c2ccccc2)C1]

Step 1: 2,2,2-Trifluoro-N-methyl-N-((2S,4R)-1-((R)-10-((2-oxo-4-phenylpyridin-1(2H)-yl)methyl)-7-azaspiro[4.5]decane-7-carbonyl)-2-phenylpiperidin-4-yl)acetamide: Prepared according to General Procedure 2 using (R)-1-((7-azaspiro[4.5]decan-10-yl)methyl)-4-phenylpyridin-2(1H)-one (30.0 mg, 0.093 mmol), 2,2,2-trifluoro-N-methyl-N-((2S,4R)-2-phenylpiperidin-4-yl)acetamide hydrochloride (30.0 mg, 0.093 mmol), triphosgene (11 mg, 0.037 mmol), pyridine (38 μL, 0.465 mmol) and DIPEA (81 μL, 0.465 mmol) in MeCN (mL) to give the title compound (26 mg, 44%). LCMS (Method B): R_{T}=1.58 min, m/z=634 [M+H]⁺.

Step 2: 1-(((R)-7-((2S,4R)-4-(Methylamino)-2-phenylpiperidine-1-carbonyl)-7-azaspiro[4.5]decan-10-yl)methyl)-4-phenylpyridin-2(1H)-one: Prepared according to General Procedure 6 using 2,2,2-trifluoro-N-methyl-N-((2S,4R)-1-((R)-10-((2-oxo-4-phenylpyridin-1(2H)-yl)methyl)-7-azaspiro[4.5]decane-7-carbonyl)-2-phenylpiperidin-4-yl)acetamide (26.0 mg, 0.041 mmol) and potassium carbonate (28.3 mg, 0.205 mmol) in MeOH (2 mL) and water (0.2 mL) to give the title compound (21 mg, 93%). LCMS (Method B): R_{T}=0.93 min, m/z=539 [M+H]⁺. ¹H NMR (500 MHz, DMSO-d₆): δ 7.75-7.69 (m, 3H), 7.52-7.43 (m, 3H), 7.25-7.16 (m, 4H), 7.16-7.10 (m, 1H), 6.66 (d, J=2.1 Hz, 1H), 6.57 (dd, J=7.1, 2.1 Hz, 1H), 4.08 (dd, J=12.7, 3.1 Hz, 1H), 3.95 (dd, J=11.7, 3.1 Hz, 1H), 3.87 (dd, J=10.7, 6.4 Hz, 1H), 3.71 (t, J=11.7 Hz, 1H), 3.41 (d, J=12.8 Hz, 1H), 3.25 (dt, J=12.1, 3.8 Hz, 1H), 2.86 (s, 1H), 2.72-2.65 (m, 2H), 2.57-2.45 (m, 2H, obscured by DMSO signal), 2.25 (s, 3H), 1.97-1.72 (m, 4H), 1.70-1.42 (m, 4H), 1.40-1.11 (m, 7H).

### Example 49: 3-((10-Amino-7-((R)-4,4,4-trifluoro-2-methylbutanoyl)-7-azaspiro[4.5]decan-10-yl)methyl)-6-phenylpyrimidin-4(3H)-one

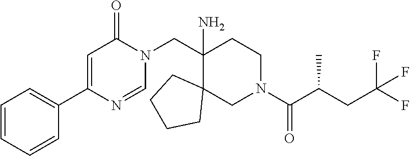 [SMILES: C[C@H](CC(F)(F)F)C(=O)N1CCC(N)(Cn2cnc(-c3ccccc3)cc2=O)C2(CCCC2)C1]

Step 1: tert-Butyl 1-((4-nitrophenyl)sulfonyl)-1,10-diazadispiro[2.0.4⁴.4³]dodecane-10-carboxylate: To tert-butyl 10-methylene-7-azaspiro[4.5]decane-7-carboxylate (4.00 g, 15.9 mmol) in dry degassed acetonitrile (70 mL) was added nosyl azide (5.45 g, 23.9 mmol) and copper(I) hexafluorophosphate (0.33 g, 1.6 mmol) under an atmosphere of argon. The reaction mixture was heated to 80° C. for 48 h. The solvents were removed in vacuo and the residue was purified by flash chromatography to give the title compound (2.50 g, 35%). LCMS (Method F): R_{T}=1.62 min, m/z=352 [M−Boc+H]⁺.

Step 2: tert-Butyl 10-((4-nitrophenyl)sulfonamido)-10-((6-oxo-4-phenylpyrimidin-1(6H)-yl)methyl)-7-azaspiro[4.5]decane-7-carboxylate: To tert-butyl 1-((4-nitrophenyl)sulfonyl)-1,10-diazadispiro[2.0.4⁴.4³]dodecane-10-carboxylate (0.60 g, 1.3 mmol) in dry DMF (10 mL) was added 6-phenylpyrimidone (0.23 g, 1.3 mmol) and the reaction mixture was heated at 85° C. for 40 h. The solvents were removed in vacuo and the residue was purified by flash chromatography to give the title compound (0.29 g, 35%). LCMS (Method F): R_{T}=4.08 min, m/z=624 [M+H]⁺.

Step 3: 4-Nitro-N-(10-((6-oxo-4-phenylpyrimidin-1(6H)-yl)methyl)-7-azaspiro[4.5]decan-10-yl)benzenesulfonamide: Prepared according to General Procedure 1 using tert-butyl 10-((4-nitrophenyl)sulfonamido)-10-((6-oxo-4-phenylpyrimidin-1(6H)-yl)methyl)-7-azaspiro[4.5]decane-7-carboxylate (34.7 mg, 0.056 mmol), DCM (1 mL) and TFA (0.5 mL), stirred at rt for 1 h to give the title compound (26.1 mg, 88%). LCMS (Method B): R_{T}=0.84 min, m/z=524 [M+H]⁺.

Step 4: 4-Nitro-N-(10-((6-oxo-4-phenylpyrimidin-1(6H)-yl)methyl)-7-((R)-4,4,4-trifluoro-2-methylbutanoyl)-7-azaspiro[4.5]decan-10-yl)benzenesulfonamide: Prepared according to General Procedure 3 using 4-nitro-N-(10-((6-oxo-4-phenylpyrimidin-1(6H)-yl)methyl)-7-azaspiro[4.5]decan-10-yl)benzenesulfonamide (26.1 mg, 0.050 mmol), (R)-4,4,4-trifluoro-2-methylbutanoic acid (7.8 mg, 0.050 mmol), HATU (19.0 mg, 0.050 mmol) and DIPEA (28 μL, 0.199 mmol) in DCM (1 mL) to give the title compound (12.0 mg, 36%). LCMS (Method B): R_{T}=1.43 min, m/z=662 [M+H]⁺.

Step 5: 3-((10-Amino-7-((R)-4,4,4-trifluoro-2-methylbutanoyl)-7-azaspiro[4.5]decan-10-yl)methyl)-6-phenylpyrimidin-4(3H)-one: To a stirred solution of 4-nitro-N-(10-((6-oxo-4-phenylpyrimidin-1(6H)-yl)methyl)-7-((R)-4,4,4-trifluoro-2-methylbutanoyl)-7-azaspiro[4.5]decan-10-yl)benzenesulfonamide (12.0 mg, 0.018 mmol) in dry MeCN (0.6 mL) was added potassium carbonate (25.1 mg, 0.182 mmol) and thiophenol (10 μL, 0.121 mmol). The reaction mixture was stirred at 50° C. for 2 h before being diluted with EtOAc and washed with saturated NaHCO_{3(aq)} solution (×2). The aqueous phase was extracted using EtOAc (×2) and the combined organics were dried (phase separator) and concentrated in vacuo. The material was purified by flash chromatography using a KP-NH column (0-100% EtOAc in cyclohexane) and freeze-dried to give the title compound (3.5 mg, 41%). LCMS (Method B): R_{T}=0.85 min, m/z=477 [M+H]⁺. ¹H NMR (500 MHz, DMSO-d₆): δ 8.64-8.59 (m, 1H), 8.12-8.04 (m, 2H), 7.53-7.46 (m, 3H), 6.99-6.93 (m, 1H), 4.54-4.45 (m, 1H), 3.78-3.38 (m, 5.5H), 3.19-3.06 (m, 1.5H), 2.79-2.67 (m, 1H), 2.32-2.20 (m, 1H), 1.87-1.77 (m, 1H), 1.73-1.52 (m, 6H), 1.44-1.29 (m, 2H), 1.20-1.01 (m, 5H).

### Example 50: 1-(((R)-7-((2S,4R)-4-((1,1-Dioxidothietan-3-yl)amino)-2-phenylpiperidine-1-carbonyl)-7-azaspiro[4.5]decan-10-yl)methyl)-4-phenylpyridin-2(1H)-one

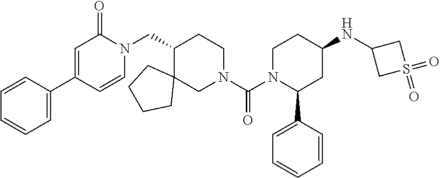 [SMILES: O=C(N1CC[C@@H](Cn2ccc(-c3ccccc3)cc2=O)C2(CCCC2)C1)N1CC[C@@H](NC2CS(=O)(=O)C2)C[C@H]1c1ccccc1]

Step 1: (R)-10-((2-Oxo-4-phenylpyridin-1(2H)-yl)methyl)-7-azaspiro[4.5]decane-7-carbonyl chloride: To a solution of triphosgene (110 mg, 0.372 mmol) in DCM (6 mL) cooled to 0° C. was added pyridine (251 μL, 3.10 mmol) and the resulting mixture stirred at 0° C. for 10 min. A solution of (R)-1-((7-azaspiro[4.5]decan-10-yl)methyl)-4-phenylpyridin-2(1H)-one (200 mg, 0.620 mmol) in DCM (6 mL) was the added dropwise to the triphosgene solution. The resulting mixture was warmed to rt and stirred for 16 h. The reaction was quenched with 1 M HCl (aq) solution and extracted with DCM (×3) using a phase separator. The combined organic phases were concentrated in vacuo to give the title compound (230 mg, 96%) that was used in the next step without purification. [Note: LCMS data not generated due to the chemical instability of the intermediate].

Step 2: 1-(((R)-7-((2S,4R)-4-((1,1-Dioxidothietan-3-yl)amino)-2-phenylpiperidine-1-carbonyl)-7-azaspiro[4.5]decan-10-yl)methyl)-4-phenylpyridin-2(1H)-one: To a solution of (R)-10-((2-oxo-4-phenylpyridin-1(2H)-yl)methyl)-7-azaspiro[4.5]decane-7-carbonyl chloride (34 mg, 0.091 mmol) and 3-(((2S,4R)-2-phenylpiperidin-4-yl)amino)thietane 1,1-dioxide hydrochloride (32.4 mg, 0.102 mmol) in MeCN (1 mL) was added DIPEA (81 μL, 0.465 mmol). The resulting mixture was stirred at rt for 18 h. The reaction was quenched with saturated NaHCO_{3(aq)} and extracted with DCM (×3) using a phase separator. The combined organic phases were concentrated in vacuo and the crude material was purified by flash chromatography using a Biotage Sfar Amino D column (10-100% EtOAc in cyclohexane; then 0-20% MeOH in EtOAc) and freeze-dried to give the title compound (16 mg, 26%). LCMS (Method B): R_{T}=1.03 min, m/z=629 [M+H]⁺. ¹H NMR (500 MHz, DMSO-d₆): δ 7.76-7.69 (m, 3H), 7.51-7.43 (m, 3H), 7.24-7.16 (m, 4H), 7.16-7.11 (m, 1H), 6.66 (d, J=2.0 Hz, 1H), 6.57 (dd, J=7.1, 2.1 Hz, 1H), 4.34-4.21 (m, 2H), 4.08 (dd, J=12.8, 3.1 Hz, 1H), 3.93 (dd, J=11.8, 3.1 Hz, 1H), 3.90-3.79 (m, 3H), 3.76-3.60 (m, 2H), 3.41 (d, J=12.8 Hz, 1H), 3.23 (dt, J=12.2, 3.7 Hz, 1H), 2.86 (s, 1H), 2.68 (d, J=13.5 Hz, 1H), 2.59-2.39 (m, 2H, signal obscured by DMSO), 1.86 (dt, J=21.3, 10.9 Hz, 3H), 1.76 (s, 1H), 1.70-1.11 (m, 12H).

### Example 51: 1-(((R)-7-((2S,4S)-4-((1,1-Dioxidothietan-3-yl)amino)-2-phenylpiperidine-1-carbonyl)-7-azaspiro[4.5]decan-10-yl)methyl)-4-phenylpyridin-2(1H)-one

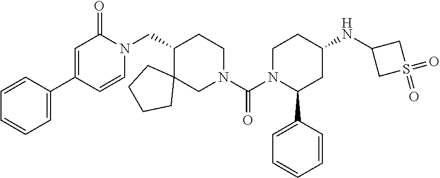 [SMILES: O=C(N1CC[C@@H](Cn2ccc(-c3ccccc3)cc2=O)C2(CCCC2)C1)N1CC[C@H](NC2CS(=O)(=O)C2)C[C@H]1c1ccccc1]

Prepared similarly to Example 50 using (R)-10-((2-oxo-4-phenylpyridin-1(2H)-yl)methyl)-7-azaspiro[4.5]decane-7-carbonyl chloride (34 mg, 0.091 mmol), 3-(((2S,4S)-2-phenylpiperidin-4-yl)amino)thietane 1,1-dioxide hydrochloride (32.4 mg, 0.102 mmol) and DIPEA (81 μL, 0.465 mmol) in MeCN (1 mL) to give the title compound (20 mg, 32%). LCMS (Method B): R_{T}=1.07 min, m/z=629 [M+H]⁺. ¹H NMR (500 MHz, DMSO-d₆): δ 7.77-7.68 (m, 3H), 7.52-7.43 (m, 3H), 7.34 (dd, J=8.2, 7.1 Hz, 2H), 7.28 (dt, J=8.4, 1.2 Hz, 2H), 7.22 (t, J=7.3 Hz, 1H), 6.66 (d, J=2.1 Hz, 1H), 6.57 (dd, J=7.1, 2.2 Hz, 1H), 4.99 (s, 1H), 4.34-4.21 (m, 2H), 4.11 (dd, J=12.6, 3.1 Hz, 1H), 3.88-3.78 (m, 2H), 3.74-3.56 (m, 3H), 3.47 (d, J=13.6 Hz, 1H), 3.26 (d, J=12.9 Hz, 1H), 2.84-2.69 (m, 2H), 2.67-2.60 (m, 1H, obscured by DMSO satellite signal), 2.44 (s, 2H), 1.87 (dq, J=11.6, 7.1 Hz, 2H), 1.75-1.11 (m, 13H).

### Example 52: 1-(((R)-7-((2S,4R)-4-Hydroxy-4-methyl-2-phenylpiperidine-1-carbonyl)-7-azaspiro[4.5]decan-10-yl)methyl)-4-phenylpyridin-2(1H)-one

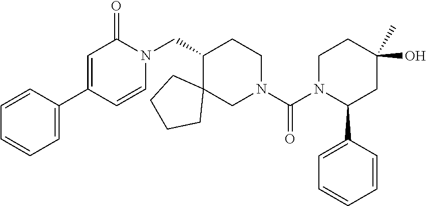 [SMILES: C[C@@]1(O)CCN(C(=O)N2CC[C@@H](Cn3ccc(-c4ccccc4)cc3=O)C3(CCCC3)C2)[C@H](c2ccccc2)C1]

Prepared according to General Procedure 7 using (R)-10-((2-oxo-4-phenylpyridin-1(2H)-yl)methyl)-7-azaspiro[4.5]decane-7-carbonyl chloride (15 mg, 0.039 mmol), (2S,4R)-4-methyl-2-phenylpiperidin-4-ol (10.7 mg, 0.047 mmol) and DIPEA (27 μL, 0.156 mmol) to give the title compound (8 mg, 37%) after lyophilisation. LCMS (Method B): R_{T}=1.30 min, m/z=540 [M+H]⁺. ¹H NMR (500 MHz, DMSO-d₆): δ 7.75-7.69 (m, 3H), 7.52-7.43 (m, 3H), 7.25-7.20 (m, 4H), 7.13 (ddd, J=8.5, 5.6, 2.3 Hz, 1H), 6.65 (d, J=2.1 Hz, 1H), 6.57 (dd, J=7.1, 2.2 Hz, 1H), 4.31 (dd, J=9.2, 4.4 Hz, 1H), 4.27 (s, 1H), 4.09 (dd, J=12.8, 3.1 Hz, 1H), 3.76 (d, J=13.1 Hz, 1H), 3.73-3.65 (m, 1H), 3.34 (d, J=12.9 Hz, 1H), 3.24 (ddd, J=12.6, 6.7, 3.8 Hz, 1H), 2.96 (ddd, J=12.3, 8.7, 3.4 Hz, 1H), 2.79 (t, J=11.9 Hz, 1H), 2.67-2.60 (m, 1H), 1.82 (ddd, J=16.3, 11.8, 8.1 Hz, 3H), 1.70-1.42 (m, 7H), 1.40-1.14 (m, 8H).

### Example 53: 4-(2-Methoxyphenyl)-1-(((R)-7-((2S,4R)-4-(methylamino)-2-phenylpiperidine-1-carbonyl)-7-azaspiro[4.5]decan-10-yl)methyl)pyridin-2(1H)-one

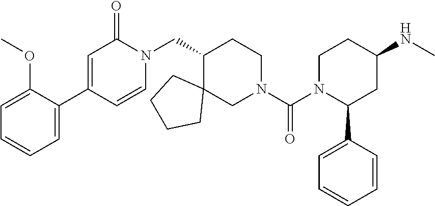 [SMILES: CN[C@@H]1CCN(C(=O)N2CC[C@@H](Cn3ccc(-c4ccccc4OC)cc3=O)C3(CCCC3)C2)[C@H](c2ccccc2)C1]

Step 1: N-((2S,4R)-1-((R)-10-((4-Chloro-2-oxopyridin-1(2H)-yl)methyl)-7-azaspiro[4.5]decane-7-carbonyl)-2-phenylpiperidin-4-yl)-2,2,2-trifluoro-N-methylacetamide: To a reaction vessel containing tert-butyl (R)-10-((4-chloro-2-oxopyridin-1(2H)-yl)methyl)-7-azaspiro[4.5]decane-7-carboxylate (300 mg, 0.788 mmol) was added 4 M HCl in 1,4-dioxane (5 mL). The reaction was stirred at rt for 1 h then concentrated in vacuo and dried in a vacuum oven at 50° C. for 18 h. Concurrently, in a separate vessel containing triphosgene (93.4 mg, 0.315 mmol) in MeCN (8 mL) at 0° C. was added pyridine (319 μL, 3.94 mmol) and the resulting mixture was stirred for 10 min before adding a solution of 2,2,2-trifluoro-N-methyl-N-((2S,4R)-2-phenylpiperidin-4-yl)acetamide hydrochloride (305 mg, 0.945 mmol) and DIPEA (179 μL, 1.02 mmol) dropwise to the triphosgene mixture. The resulting reaction stirred at rt for 18 h. The reaction was then transferred to the vessel containing the vacuum-dried residue and DIPEA (509 μL, 2.91 mmol) was added. After 40 h stirring at rt, the reaction was quenched with 0.5 M HCl_{(aq)} and extracted with DCM (×3) using a phase separator. The combined organic phases were concentrated in vacuo and the crude material was purified by flash chromatography (10-100% EtOAc in cyclohexane) to give the title compound (376 mg, 80%). LCMS (Method B): R_{T}=1.49 min, m/z=593, 595 [M+H]⁺.

Step 2: 2,2,2-Trifluoro-N-((2S,4R)-1-((R)-10-((4-(2-methoxyphenyl)-2-oxopyridin-1(2H)-yl)methyl)-7-azaspiro[4.5]decane-7-carbonyl)-2-phenylpiperidin-4-yl)-N-methylacetamide: Prepared according to General Procedure 5 using N-((2S,4R)-1-((R)-10-((4-chloro-2-oxopyridin-1(2H)-yl)methyl)-7-azaspiro[4.5]decane-7-carbonyl)-2-phenylpiperidin-4-yl)-2,2,2-trifluoro-N-methylacetamide (50 mg, 0.084 mmol), (2-methoxyphenyl)boronic acid (25.6 mg, 0.169 mmol), Na₂CO₃ (26.8 mg, 0.253 mmol) and PdCl₂(dppf)·DCM (3.57 mg, 0.004 mmol) in 1,4-dioxane (0.6 mL) and water (0.2 mL) to give the title compound (41 mg, 73%). LCMS (Method B): R_{T}=1.56 min, m/z=665 [M+H]⁺.

Step 3: 4-(2-Methoxyphenyl)-1-(((R)-7-((2S,4R)-4-(methylamino)-2-phenylpiperidine-1-carbonyl)-7-azaspiro[4.5]decan-10-yl)methyl)pyridin-2(1H)-one: Prepared according to General Procedure 6 using 2,2,2-trifluoro-N-((2S,4R)-1-((R)-10-((4-(2-methoxyphenyl)-2-oxopyridin-1(2H)-yl)methyl)-7-azaspiro[4.5]decane-7-carbonyl)-2-phenylpiperidin-4-yl)-N-methylacetamide (41.0 mg, 0.062 mmol) and potassium carbonate (42.6 mg, 0.308 mmol) in MeOH (2 mL) and water (0.2 mL) to give the title compound (26 mg, 73%) as a colourless solid. LCMS (Method B): R_{T}=0.94 min, m/z=569 [M+H]⁺. ¹H NMR (500 MHz, DMSO-d₆): δ 7.61 (d, J=7.0 Hz, 1H), 7.41 (ddd, J=8.3, 7.4, 1.8 Hz, 1H), 7.34 (dd, J=7.6, 1.7 Hz, 1H), 7.24-7.17 (m, 4H), 7.16-7.10 (m, 2H), 7.03 (td, J=7.5, 1.0 Hz, 1H), 6.45 (d, J=1.9 Hz, 1H), 6.36 (dd, J=7.0, 2.0 Hz, 1H), 4.06 (dd, J=12.7, 3.0 Hz, 1H), 3.95 (dd, J=11.8, 3.1 Hz, 1H), 3.92-3.83 (m, 1H), 3.80 (s, 3H), 3.68 (t, J=11.8 Hz, 1H), 3.41 (d, J=12.8 Hz, 1H), 3.25 (dt, J=12.1, 3.8 Hz, 1H), 2.88 (s, 1H), 2.72-2.65 (m, 2H), 2.43 (tt, J=10.6, 4.0 Hz, 1H), 2.24 (s, 3H), 1.96-1.72 (m, 4H), 1.71-1.42 (m, 5H), 1.40-1.11 (m, 7H).

### Example 54: 1-(((R)-7-((2S,4R)-4-(Methylamino)-2-phenylpiperidine-1-carbonyl)-7-azaspiro[4.5]decan-10-yl)methyl)-4-(o-tolyl)pyridin-2(1H)-one

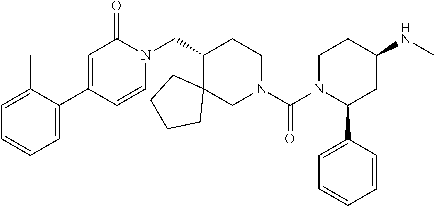 [SMILES: CN[C@@H]1CCN(C(=O)N2CC[C@@H](Cn3ccc(-c4ccccc4C)cc3=O)C3(CCCC3)C2)[C@H](c2ccccc2)C1]

Step 1: 2,2,2-Trifluoro-N-methyl-N-((2S,4R)-1-((R)-10-((2-oxo-4-(o-tolyl)pyridin-1(2H)-yl)methyl)-7-azaspiro[4.5]decane-7-carbonyl)-2-phenylpiperidin-4-yl)acetamide: Prepared according to General Procedure 5 using N-((2S,4R)-1-((R)-10-((4-chloro-2-oxopyridin-1(2H)-yl)methyl)-7-azaspiro[4.5]decane-7-carbonyl)-2-phenylpiperidin-4-yl)-2,2,2-trifluoro-N-methylacetamide (50 mg, 0.084 mmol), o-tolylboronic acid (22.9 mg, 0.169 mmol), Na₂CO₃ (26.8 mg, 0.253 mmol) and PdCl₂(dppf)·DCM (3.57 mg, 0.004 mmol) in 1,4-dioxane (0.6 mL) and water (0.2 mL) to give the title compound (40 mg, 73%). LCMS (Method B): R_{T}=1.62 min, m/z=649 [M+H]⁺.

Step 2: 1-(((R)-7-((2S,4R)-4-(Methylamino)-2-phenylpiperidine-1-carbonyl)-7-azaspiro[4.5]decan-10-yl)methyl)-4-(o-tolyl)pyridin-2(1H)-one: Prepared according to General Procedure 6 using 2,2,2-trifluoro-N-methyl-N-((2S,4R)-1-((R)-10-((2-oxo-4-(o-tolyl)pyridin-1(2H)-yl)methyl)-7-azaspiro[4.5]decane-7-carbonyl)-2-phenylpiperidin-4-yl)acetamide (40.0 mg, 0.062 mmol) and potassium carbonate (42.6 mg, 0.308 mmol) in MeOH (2 mL) and water (0.2 mL) to give the title compound (22 mg, 63%). LCMS (Method B): R_{T}=0.96 min, m/z=553 [M+H]⁺. ¹H NMR (500 MHz, DMSO-d₆): δ 7.68 (d, J=6.9 Hz, 1H), 7.34-7.17 (m, 8H), 7.16-7.11 (m, 1H), 6.27 (d, J=1.9 Hz, 1H), 6.22 (dd, J=6.9, 2.0 Hz, 1H), 4.09 (dd, J=12.8, 3.1 Hz, 1H), 3.96 (dd, J=11.7, 3.1 Hz, 1H), 3.88 (dd, J=11.0, 6.4 Hz, 1H), 3.72 (t, J=11.8 Hz, 1H), 3.40 (d, J=12.9 Hz, 1H), 3.28-3.22 (m, 1H), 2.90 (s, 1H), 2.74-2.66 (m, 2H), 2.56-2.38 (m, 2H, obscured by DMSO signal), 2.27 (s, 3H), 2.26 (s, 3H), 1.97-1.82 (m, 3H), 1.76 (s, 1H), 1.70-1.43 (m, 4H), 1.41-1.11 (m, 7H).

### Example 55: 4-(2-Fluorophenyl)-1-(((R)-7-((2S,4R)-4-(methylamino)-2-phenylpiperidine-1-carbonyl)-7-azaspiro[4.5]decan-10-yl)methyl)pyridin-2(1H)-one

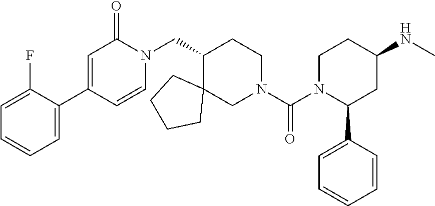 [SMILES: CN[C@@H]1CCN(C(=O)N2CC[C@@H](Cn3ccc(-c4ccccc4F)cc3=O)C3(CCCC3)C2)[C@H](c2ccccc2)C1]

Step 1: 2,2,2-Trifluoro-N-((2S,4R)-1-((R)-10-((4-(2-fluorophenyl)-2-oxopyridin-1(2H)-yl)methyl)-7-azaspiro[4.5]decane-7-carbonyl)-2-phenylpiperidin-4-yl)-N-methylacetamide: Prepared according to General Procedure 5 using N-((2S,4R)-1-((R)-10-((4-chloro-2-oxopyridin-1(2H)-yl)methyl)-7-azaspiro[4.5]decane-7-carbonyl)-2-phenylpiperidin-4-yl)-2,2,2-trifluoro-N-methylacetamide (50 mg, 0.084 mmol), (2-fluorophenyl)boronic acid (23.6 mg, 0.169 mmol), Na₂CO₃ (26.8 mg, 0.253 mmol) and PdCl₂(dppf)·DCM (3.57 mg, 0.004 mmol) in 1,4-dioxane (0.6 mL) and water (0.2 mL) to give the title compound (50 mg, 91%). LCMS (Method B): R_{T}=1.56 min, m/z=653 [M+H]⁺.

Step 2: 4-(2-Fluorophenyl)-1-(((R)-7-((2S,4R)-4-(methylamino)-2-phenylpiperidine-1-carbonyl)-7-azaspiro[4.5]decan-10-yl)methyl)pyridin-2(1H)-one: Prepared according to General Procedure 6 using 2,2,2-trifluoro-N-((2S,4R)-1-((R)-10-((4-(2-fluorophenyl)-2-oxopyridin-1(2H)-yl)methyl)-7-azaspiro[4.5]decane-7-carbonyl)-2-phenylpiperidin-4-yl)-N-methylacetamide (50.0 mg, 0.077 mmol) and potassium carbonate (52.9 mg, 0.383 mmol) in MeOH (2 mL) and water (0.2 mL) to give the title compound (18.7 mg, 43%). LCMS (Method B): R_{T}=0.95 min, m/z=557 [M+H]⁺. ¹H NMR (500 MHz, DMSO-d₆): δ 7.73 (d, J=7.1 Hz, 1H), 7.59 (td, J=7.8, 1.8 Hz, 1H), 7.50 (dddd, J=8.5, 7.2, 5.2, 1.8 Hz, 1H), 7.37-7.29 (m, 2H), 7.24-7.17 (m, 4H), 7.15-7.11 (m, 1H), 6.54 (t, J=1.5 Hz, 1H), 6.42 (dt, J=7.0, 2.0 Hz, 1H), 4.09 (dd, J=12.7, 3.1 Hz, 1H), 3.95 (dd, J=11.7, 3.1 Hz, 1H), 3.88 (dd, J=11.1, 6.5 Hz, 1H), 3.71 (t, J=11.8 Hz, 1H), 3.41 (d, J=12.8 Hz, 1H), 3.25 (dt, J=12.1, 3.8 Hz, 1H), 2.87 (s, 1H), 2.70-2.64 (m, 2H), 2.56-2.39 (m, 2H, obscured by DMSO signal), 2.24 (s, 3H), 1.96-1.71 (m, 4H), 1.70-1.42 (m, 4H), 1.40-1.12 (m, 7H).

### Example 56: 3-(((R)-7-((2S,4R)-4-(Methylamino)-2-phenylpiperidine-1-carbonyl)-7-azaspiro[4.5]decan-10-yl)methyl)-6-phenylpyrimidin-4(3H)-one

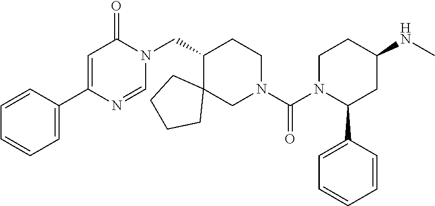 [SMILES: CN[C@@H]1CCN(C(=O)N2CC[C@@H](Cn3cnc(-c4ccccc4)cc3=O)C3(CCCC3)C2)[C@H](c2ccccc2)C1]

Step 1: tert-Butyl 10-((6-oxo-4-phenylpyrimidin-1(6H)-yl)methyl)-7-azaspiro[4.5]decane-7-carboxylate: Prepared according to General Procedure 4 using 6-phenylpyrimidone (6.00 g, 35 mmol), tert-butyl 10-(bromomethyl)-7-azaspiro[4.5]decane-7-carboxylate (12.8 g, 38 mmol) and cesium carbonate (22.7 g, 70 mmol) to give the title compound (7.30 g, 50%). LCMS (Method F): R_{T}=1.49 min. m/z=368 [M−butene+H]⁺.

Step 2: tert-Butyl (R)-10-((6-oxo-4-phenylpyrimidin-1(6H)-yl)methyl)-7-azaspiro[4.5]decane-7-carboxylate and tert-butyl (S)-10-((6-oxo-4-phenylpyrimidin-1(6H)-yl)methyl)-7-azaspiro[4.5]decane-7-carboxylate: tert-Butyl 10-((6-oxo-4-phenylpyrimidin-1(6H-yl)methyl)-7-azaspiro[4.5]decane-7-carboxylate (7.30 g) was resolved into the single stereoisomers by chiral HPLC using a Chiralpak IB (30 mm×250 mm, 5 μm) column with isocratic solvent conditions: 80:20 hexane/IPA. The first eluted material afforded tert-butyl (R)-10-((6-oxo-4-phenylpyrimidin-1(6H)-yl)methyl)-7-azaspiro[4.5]decane-7-carboxylate (3.30 g). [α]_{D}²⁵=+69.3 (c 0.25 in MeOH). The second eluted material afforded tert-butyl (S)-10-((6-oxo-4-phenylpyrimidin-1(6H)-yl)methyl)-7-azaspiro[4.5]decane-7-carboxylate (3.12 g). [α]_{D}²⁵=−72.4 (c 0.25 in MeOH).

Step 3: (R)-3-((7-Azaspiro[4.5]decan-10-yl)methyl)-6-phenylpyrimidin-4(3H)-one: Prepared according to General Procedure 1 using tert-butyl (R)-10-((6-oxo-4-phenylpyrimidin-1(6H-yl)methyl)-7-azaspiro[4.5]decane-7-carboxylate (1000 mg, 2.36 mmol), DCM (20 mL) and TFA (10 mL), stirred at rt for 1 h to give the title compound (764 mg, quantitative). LCMS (Method B): R_{T}=0.716 min, m/z=324 [M+H]⁺.

Step 4: 2,2,2-Trifluoro-N-methyl-N-((2S,4R)-1-((R)-10-((6-oxo-4-phenylpyrimidin-1(6H)-yl)methyl)-7-azaspiro[4.5]decane-7-carbonyl)-2-phenylpiperidin-4-yl)acetamide: Prepared according to General Procedure 2 using (R)-3-((7-azaspiro[4.5]decan-10-yl)methyl)-6-phenylpyrimidin-4(3H)-one (50 mg, 0.155 mmol), 2,2,2-trifluoro-N-methyl-N-((2S,4R)-2-phenylpiperidin-4-yl)acetamide hydrochloride (50.0 mg, 0.155 mmol), triphosgene (18.4 mg, 0.062 mmol), pyridine (63 μL, 0.773 mmol) and DIPEA (108 μL, 0.773 mmol) in dry MeCN (1 mL) to give the title compound (53.5 mg, 54%). LCMS (Method A): R_{T}=1.75 min, m/z=636 [M+H]⁺.

Step 5: 3-(((R)-7-((2S,4R)-4-(Methylamino)-2-phenylpiperidine-1-carbonyl)-7-azaspiro[4.5]decan-10-yl)methyl)-6-phenylpyrimidin-4(3H)-one: Prepared according to General Procedure 6 using 2,2,2-trifluoro-N-methyl-N-((2S,4R)-1-((R)-10-((6-oxo-4-phenylpyrimidin-1(6H)-yl)methyl)-7-azaspiro[4.5]decane-7-carbonyl)-2-phenylpiperidin-4-yl)acetamide (53.5 mg, 0.084 mmol) and potassium carbonate (58.2 mg, 0.421 mmol) in a solution of MeOH (3 mL) and water (0.3 mL). The crude residue was purified by flash chromatography using a KP-NH cartridge (0-100% EtOAc in cyclohexane; then 0-20% MeOH in EtOAc) and freeze-dried to give the title compound (27.4 mg, 59%). LCMS (Method B): R_{T}=0.905 min, m/z=540 [M+H]⁺. ¹H NMR (500 MHz, DMSO-d₆): δ 8.55 (s, 1H), 8.09-8.03 (m, 2H), 7.52-7.46 (m, 3H), 7.25-7.10 (m, 5H), 6.97 (s, 1H), 4.13-4.07 (m, 1H), 3.98-3.92 (m, 1H), 3.91-3.84 (m, 1H), 3.75-3.66 (m, 1H), 3.48-3.39 (m, 1H), 2.89-2.76 (m, 1H), 2.68-2.61 (m, 2H), 2.48-2.40 (m, 1H), 2.24 (s, 3H), 1.96-1.43 (m, 9H), 1.39-1.13 (m, 8H).

### Example 57: 3-((7-((2S,4R)-4-Amino-2-phenylpiperidine-1-carbonyl)-10-fluoro-7-azaspiro[4.5]decan-10-yl)methyl)-6-phenylpyrimidin-4(3H)-one

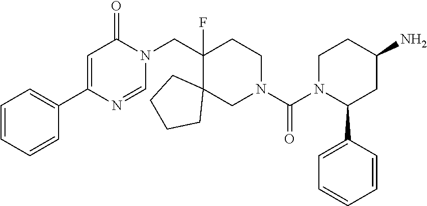 [SMILES: N[C@@H]1CCN(C(=O)N2CCC(F)(Cn3cnc(-c4ccccc4)cc3=O)C3(CCCC3)C2)[C@H](c2ccccc2)C1]

Step 1: tert-Butyl 10-fluoro-10-((tosyloxy)methyl)-7-azaspiro[4.5]decane-7-carboxylate: To tert-butyl 1-oxa-10-azadispiro[2.0.4⁴.4³]dodecane-10-carboxylate (9.00 g, 33.7 mmol) [prepared according to WO2020115501 (p 99, Epoxide 2)] in benzene (300 mL) at 0° C. was added boron fluoride diethyl etherate (4.78 g, 33.7 mmol) and stirred for 2 h. To the reaction mixture was added triethylamine (17.0 g, 168 mmol) and p-toluenesulfonyl chloride (7.70 g, 40.4 mmol). After 24 h, the reaction mixture was filtered and evaporated to dryness. The remaining residue was purified by flash chromatography to give the title compound (5.00 g, 34%). LCMS (Method F): R_{T}=1.59 min, m/z=386 [M−butene+H]⁺.

Step 2: tert-Butyl 10-fluoro-10-((6-oxo-4-phenylpyrimidin-1(6H)-yl)methyl)-7-azaspiro[4.5]decane-7-carboxylate: To 6-phenylpyrimidin-4(3H)-one (0.50 g, 2.9 mmol) in DMF (25 mL) was added tert-butyl 10-fluoro-10-((tosyloxy)methyl)-7-azaspiro[4.5]decane-7-carboxylate (1.41 g, 3.2 mmol) and cesium carbonate (1.89 g, 5.8 mmol) and the resulting mixture was stirred at 120° C. for 48 h. After cooling to rt, the reaction mixture was filtered and evaporated to dryness. The crude material was purified by preparative HPLC to give the title compound (0.044 g, 3.4%). LCMS (Method F): R_{T}=1.45 min, m/z=386 [M−butene+H]⁺.

Step 3: 3-((10-Fluoro-7-azaspiro[4.5]decan-10-yl)methyl)-6-phenylpyrimidin-4(3H)-one: Prepared according to General Procedure 1 using tert-butyl 10-fluoro-10-((6-oxo-4-phenylpyrimidin-1(6H)-yl)methyl)-7-azaspiro[4.5]decane-7-carboxylate (44 mg, 0.100 mmol) and 4 M HCl in 1,4-dioxane (2 mL) to give the title compound (37 mg, 98%). LCMS (Method B): R_{T}=0.77 min. m/z=322 [M−butene+H]⁺.

Step 4: tert-Butyl ((2S,4R)-1-(10-fluoro-10-((6-oxo-4-phenylpyrimidin-1(6H)-yl)methyl)-7-azaspiro[4.5]decane-7-carbonyl)-2-phenylpiperidin-4-yl)carbamate: Prepared according to General Procedure 2 using 3-((10-fluoro-7-azaspiro[4.5]decan-10-yl)methyl)-6-phenylpyrimidin-4(3H)-one (14 mg, 0.037 mmol), tert-butyl ((2S,4R)-2-phenylpiperidin-4-yl)carbamate (11.3 mg, 0.041 mmol), triphosgene (3.85 mg, 0.013 mmol), pyridine (15.0 μL, 0.185 mmol) and DIPEA (32.4 μL, 0.185 mmol) in MeCN (1 mL) to give the title compound (10 mg, 42%). LCMS (Method B): R_{T}=1.62 min, m/z=644 [M+H]⁺.

Step 5: 3-((7-((2S,4R)-4-Amino-2-phenylpiperidine-1-carbonyl)-10-fluoro-7-azaspiro[4.5]decan-10-yl)methyl)-6-phenylpyrimidin-4(3H)-one: Prepared according to General Procedure 1 using tert-butyl ((2S,4R)-1-(10-fluoro-10-((6-oxo-4-phenylpyrimidin-1(6H)-yl)methyl)-7-azaspiro[4.5]decane-7-carbonyl)-2-phenylpiperidin-4-yl)carbamate (10 mg, 0.016 mmol) and 4 M HCl in 1,4-dioxane (1 mL) to give the title compound (2.5 mg, 26%). LCMS (Method B): R_{T}=0.99 min, m/z=544 [M+H]⁺. ¹H NMR (500 MHz, DMSO-d₆): 8.45 (d, J=2.2 Hz, 1H), 8.14-8.06 (m, 2H), 7.51 (qd, J=4.3, 1.6 Hz, 3H), 7.26-7.17 (m, 4H), 7.17-7.12 (m, 1H), 7.04 (d, J=0.8 Hz, 1H), 4.63 (dd, J=35.9, 14.4 Hz, 1H), 4.12 (dd, J=14.4, 10.2 Hz, 1H), 3.95 (dd, J=11.7, 3.1 Hz, 1H), 3.81-3.70 (m, 1H), 3.26-3.18 (m, 2H), 3.11 (d, J=13.5 Hz, 1H), 2.75-2.63 (m, 2H (obscured by DMSO satellite)), 1.96-1.32 (m, 13H), 1.26 (q, J=12.0 Hz, 2H), 1.09-1.00 (m, 1H). [NH signal not fully observed].

### Example 58: 1-(((S)-10-Methoxy-7-((2S,4R)-4-(methylamino)-2-phenylpiperidine-1-carbonyl)-7-azaspiro[4.5]decan-10-yl)methyl)-4-phenylpyridin-2(1H)-one

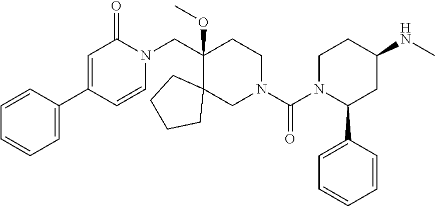 [SMILES: CN[C@@H]1CCN(C(=O)N2CC[C@](Cn3ccc(-c4ccccc4)cc3=O)(OC)C3(CCCC3)C2)[C@H](c2ccccc2)C1]

Step 1: tert-Butyl (S)-10-((4-chloro-2-oxopyridin-1(2H)-yl)methyl)-10-methoxy-7-azaspiro[4.5]decane-7-carboxylate: To a stirring solution of tert-butyl (S)-10-((4-chloro-2-oxopyridin-1(2H)-yl)methyl)-10-hydroxy-7-azaspiro[4.5]decane-7-carboxylate (500 mg, 1.26 mmol) [prepared according to WO2020115501 (p 263, Example 287, Step 1)] in DMF (10 mL) cooled to 0° C. was added sodium hydride (60% dispersion in mineral oil, 60.5 mg, 1.51 mmol) portionwise and the resulting mixture was stirred at 0° C. for 1 h. Iodomethane (118 μL, 1.89 mmol) was added and the resulting mixture was allowed to warm to rt overnight while stirring. The reaction mixture was diluted with EtOAc and washed with 1:1 water/brine mix (×3), then washed with brine, dried (MgSO₄), filtered, and concentrated in vacuo. The crude residue was purified by flash chromatography (0-60% EtOAc in cyclohexane) to give the title compound (365 mg, 70%). LCMS (Method A): R_{T}=1.76 min, m/z=355, 357 [M−butene+H]⁺.

Step 2: tert-Butyl (S)-10-methoxy-10-((2-oxo-4-phenylpyridin-1(2H)-yl)methyl)-7-azaspiro[4.5]decane-7-carboxylate: Prepared according to General Procedure 5 using tert-butyl (S)-10-((4-chloro-2-oxopyridin-1(2H)-yl)methyl)-10-methoxy-7-azaspiro[4.5]decane-7-carboxylate (200 mg, 0.487 mmol), phenylboronic acid (119 mg, 0.973 mmol), Na₂CO₃ (155 mg, 1.46 mmol) and PdCl₂(dppf)·DCM (20.6 mg, 0.024 mmol) in 1,4-dioxane (3 mL) and water (1 mL) to give the title compound (220 mg, quantitative). LCMS (Method A): R_{T}=1.87 min, m/z=453 [M+H]⁺.

Step 3: (S)-1-((10-Methoxy-7-azaspiro[4.5]decan-10-yl)methyl)-4-phenylpyridin-2(1H)-one: Prepared according to General Procedure 1 using tert-butyl (S)-10-methoxy-10-((2-oxo-4-phenylpyridin-1(2H)-yl)methyl)-7-azaspiro[4.5]decane-7-carboxylate (220 mg, 0.486 mmol) and TFA (1 mL) in DCM (3 mL) to give the title compound (170 mg, 99%). LCMS (Method A): R_{T}=0.72 min, m/z=353 [M+H]⁺.

Step 4: 2,2,2-Trifluoro-N-((2S,4R)-1-((S)-10-methoxy-10-((2-oxo-4-phenylpyridin-1(2H)-yl)methyl)-7-azaspiro[4.5]decane-7-carbonyl)-2-phenylpiperidin-4-yl)-N-methylacetamide: Prepared according to General Procedure 2 using (S)-1-((10-methoxy-7-azaspiro[4.5]decan-10-yl)methyl)-4-phenylpyridin-2(1H)-one (30 mg, 0.085 mmol), 2,2,2-trifluoro-N-methyl-N-((2S,4R)-2-phenylpiperidin-4-yl)acetamide hydrochloride (30.2 mg, 0.094 mmol), triphosgene (8.8 mg, 0.030 mmol), pyridine (34 μL, 0.426 mmol) and DIPEA (74 μL, 0.426 mmol) in MeCN (2 mL) to give the title compound (41 mg, 72%). LCMS (Method B): R_{T}=1.63 min, m/z=665 [M+H]⁺.

Step 5: 1-(((S)-10-Methoxy-7-((2S,4R)-4-(methylamino)-2-phenylpiperidine-1-carbonyl)-7-azaspiro[4.5]decan-10-yl)methyl)-4-phenylpyridin-2(1H)-one: Prepared according to General Procedure 6 using 2,2,2-trifluoro-N-((2S,4R)-1-((S)-10-methoxy-10-((2-oxo-4-phenylpyridin-1(2H)-yl)methyl)-7-azaspiro[4.5]decane-7-carbonyl)-2-phenylpiperidin-4-yl)-N-methylacetamide (41 mg, 0.062 mmol) and K₂CO₃ (42.6 mg, 0.308 mmol) in MeOH (2 mL) and water (0.2 mL) to give the title compound (21 mg, 60%). LCMS (Method B): R_{T}=01 1.02 min, m/z=569 [M+H]⁺. ¹H NMR (500 MHz, DMSO-d₆): b 7.77-7.72 (m, 2H), 7.58 (d, J=7.2 Hz, 1H), 7.53-7.44 (m, 3H), 7.26-7.17 (m, 4H), 7.16-7.10 (m, 1H), 6.70 (d, J=2.1 Hz, 1H), 6.62 (dd, J=7.2, 2.2 Hz, 1H), 4.48 (d, J=14.1 Hz, 1H), 4.01-3.90 (m, 2H), 3.76-3.47 (m, 2H), 3.26-3.20 (m, 1H), 3.15 (s, 3H), 3.04-2.93 (m, 1H), 2.69-2.64 (m, 1H), 2.47-2.39 (m, 2H), 2.25 (s, 3H), 1.92 (t, J=12.9 Hz, 2H), 1.74-1.26 (m, 10H), 1.25-1.14 (m, 2H), 0.90 (s, 1H).

The following table contains further Examples that were prepared using similar chemistry procedures to those outlined in the previous Examples. Examples 59, 60 and 61 were prepared similarly to Examples 53, 54 and 55, respectively, except using Intermediate 1 instead of Intermediate 6 and therefore, using General Procedure 1 instead of General Procedure 6 for the final deprotection step in all cases. Example 62 was prepared similarly to Example 56 except using Intermediate 5 instead of Intermediate 6 and therefore, using General Procedure 1 instead of General Procedure 6 for the final deprotection step. Example 63 and Example 64 were prepared similarly to Example 56 and Example 48, respectively, except using 2,2,2-trifluoro-N-((2S,4R)-2-(3-fluorophenyl)piperidin-4-yl)-N-methylacetamide hydrochloride (Intermediate 10) instead of 2,2,2-trifluoro-N-methyl-N-((2S,4R)-2-phenylpiperidin-4-yl)acetamide hydrochloride. Example 65 and Example 66 were prepared similarly to Example 48 and Example 56, respectively, except using 2,2,2-trifluoro-N-((2S,4R)-2-(3-fluorophenyl)piperidin-4-yl)acetamide hydrochloride (Intermediate 11) instead of 2,2,2-trifluoro-N-methyl-N-((2S,4R)-2-phenylpiperidin-4-yl)acetamide hydrochloride. Example 67 was prepared similarly to Example 45 except using Intermediate 12 instead of (R)-1-((7-azaspiro[4.5]decan-10-yl)methyl)-4-phenylpyridin-2(1H)-one and Intermediate 1 instead of Intermediate 5. Example 68 was prepared similarly to Example 45 except using Intermediate 13 instead of Intermediate 5 and using General Procedure 10 for the protecting group removal. Example 69 was prepared similarly to Example 45 except using Intermediate 14 and converting to tert-butyl (R)-3,3-dimethyl-4-((2-oxo-4-phenylpyridin-1(2H)-yl)methyl)piperidine-1-carboxylate using phenylboronic acid and General Procedure 5 instead of tert-butyl (R)-10-((2-oxo-4-phenylpyridin-1(2H)-yl)methyl)-7-azaspiro[4.5]decane-7-carboxylate and using Intermediate 1 instead of Intermediate 5. Example 70 was prepared similarly to Example 56 except using Intermediate 15 instead of Intermediate 6 and therefore, using General Procedure 1 instead of General Procedure 6 for the final deprotection step. Example 71 was prepared similarly to Example 56 except using Intermediate 16 instead of Intermediate 6 and no final deprotection step was required. Example 72 was prepared similarly to Example 69 except using Intermediate 6 and General Procedure 6 instead of Intermediate 1 and General Procedure 1. Example 73 was prepared similarly to Example 58 except using Intermediate and General Procedure 1 instead of Intermediate 6 and General Procedure 6. Example 74 was prepared similarly to Example 9 except using Intermediate 10 and General Procedure 6 instead of Intermediate 1 and General Procedure 1. Example 75 was prepared similarly to Example 9 except using Intermediate 5. Example 76 was prepared similarly to Example 56 except using Intermediate 17 instead of Intermediate 6 and no final deprotection step was required. Example 77 was prepared similarly to Example 45 except using Intermediate 18 instead of tert-butyl (R)-10-((2-oxo-4-phenylpyridin-1 (2H)-yl)methyl)-7-azaspiro[4.5]decane-7-carboxylate and using Intermediate 1 instead of Intermediate 5. Example 78 was prepared similarly to Example 56 except using Intermediate 19 instead of Intermediate 6. Example 79 was prepared similarly to Example 62 except using Intermediate 20 instead of Intermediate 5. Example 80 was prepared similarly to Example 66 except using Intermediate 21 instead of Intermediate 11. Example 81 was prepared similarly to Example 77 except using Intermediate 11 and General Procedure 6 instead of Intermediate 1 and General Procedure 1. Example 82 was prepared similarly to Example 81 except using Intermediate 22 instead of Intermediate 11. Example 83 was prepared similarly to Example 67 except using Intermediate 6 and General Procedure 6 instead of Intermediate 1 and General Procedure 1. Example 84 was prepared similarly to Example 81 except using Intermediate 6 instead of Intermediate 11.

### Example 85: 1-(((R)-7-((2S,4S,5R)-4-Amino-5-fluoro-2-phenylpiperidine-1-carbonyl)-7-azaspiro[4.5]decan-10-yl)methyl)-4-phenylpyridin-2(1H)-one and 1-(((R)-7-((2S,4S,5S)-4-Amino-5-fluoro-2-phenylpiperidine-1-carbonyl)-7-azaspiro[4.5]decan-10-yl)methyl)-4-phenylpyridin-2(1H)-one

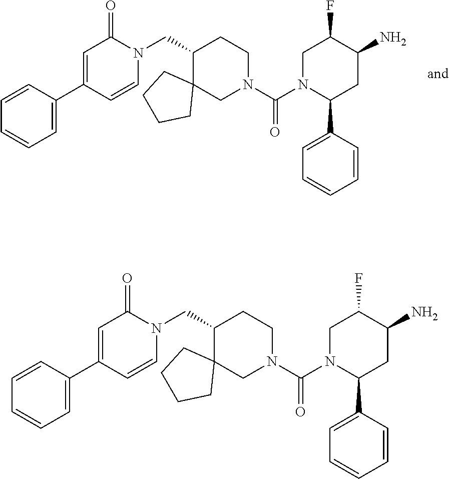 [SMILES: N[C@H]1C[C@@H](c2ccccc2)N(C(=O)N2CC[C@@H](Cn3ccc(-c4ccccc4)cc3=O)C3(CCCC3)C2)C[C@H]1F ; N[C@H]1C[C@@H](c2ccccc2)N(C(=O)N2CC[C@@H](Cn3ccc(-c4ccccc4)cc3=O)C3(CCCC3)C2)C[C@@H]1F]

Step 1: tert-Butyl (2S,4S)-4-amino-5-fluoro-2-phenylpiperidine-1-carboxylate: To a stirred solution of tert-butyl (2S,4S)-4-azido-5-fluoro-2-phenylpiperidine-1-carboxylate (80 mg, 0.250 mmol) [Intermediate 23] in THF (3 mL) and water (1 mL) was added triphenylphosphine (72 mg, 0.275 mmol). The resulting mixture was stirred at 40° C. for 18 h. The reaction mixture was concentrated in vacuo and the remaining residue was purified by flash chromatography using a KP-NH column (5-100% EtOAc in cyclohexane; then 0-20% MeOH in EtOAc) to give the title compound (65 mg, 88%). LCMS (Method B): R_{T}=0.79 min. m/z=239 [M−butene+H]P.

Step 2: tert-Butyl (2S,4S)-5-fluoro-2-phenyl-4-(2,2,2-trifluoroacetamido)piperidine-1-carboxylate: To a stirred solution of tert-butyl (2S,4S)-4-amino-5-fluoro-2-phenylpiperidine-1-carboxylate (100 mg, 0.340 mmol) and trifluoroacetic anhydride (58 μL, 0.408 mmol) in DCM (1.7 mL) at 0° C. were added DIPEA (0.24 mL, 1.36 mmol) and pyridine (82 μL, 1.02 mmol). The reaction was warmed to rt and stirred for 1.5 h. The reaction was quenched with saturated NaHCO₃ (aq) solution and extracted with DCM (×3) using a phase separator. The combined organic phase was concentrated in vacuo to give the title compound (130 mg, quantitative). LCMS (Method B): R_{T}=1.43 min, m/z=291 [M−Boc+H]⁺.

Step 3: 2,2,2-Trifluoro-N-((2S,4S)-5-fluoro-2-phenylpiperidin-4-yl)acetamide: Prepared according to General Procedure 1 using tert-butyl (2S,4S)-5-fluoro-2-phenyl-4-(2,2,2-trifluoroacetamido)piperidine-1-carboxylate (130 mg, 0.333 mmol) and TFA (1 mL) in DCM (3 mL) to give the title compound (90 mg, 93%). LCMS (Method B): R_{T}=0.56 min, 0.62 min, m/z=291 [M+H]⁺. [Note: LCMS suggests 1:1 mixture of stereoisomers].

Step 4: 2,2,2-Trifluoro-N-((2S,4S)-5-fluoro-1-((R)-10-((2-oxo-4-phenylpyridin-1(2H)-yl)methyl)-7-azaspiro[4.5]decane-7-carbonyl)-2-phenylpiperidin-4-yl)acetamide: Prepared according to General Procedure 2 using (R)-1-((7-azaspiro[4.5]decan-10-yl)methyl)-4-phenylpyridin-2(1H-one (25 mg, 0.078 mmol), 2,2,2-trifluoro-N-((2S,4S)-5-fluoro-2-phenylpiperidin-4-yl)acetamide (27 mg, 0.093 mmol), triphosgene (9.2 mg, 0.031 mmol), pyridine (31 μL, 0.388 mmol) and DIPEA (68 μL, 0.388 mmol) in MeCN (2 mL) to give the title compound (10 mg, 20%). LCMS (Method B): R_{T}=1.47 min, m/z=639 [M+H]⁺.

Step 5: 1-(((R)-7-((2S,4S,5R)-4-Amino-5-fluoro-2-phenylpiperidine-1-carbonyl)-7-azaspiro[4.5]decan-10-yl)methyl)-4-phenylpyridin-2(1H)-one and 1-(((R)-7-((2S,4S,5S)-4-Amino-5-fluoro-2-phenylpiperidine-1-carbonyl)-7-azaspiro[4.5]decan-10-yl)methyl)-4-phenylpyridin-2(1H)-one: Prepared according to General Procedure 6 using 2,2,2-trifluoro-N-((2S,4S)-5-fluoro-1-((R)-10-((2-oxo-4-phenylpyridin-1(2H)-yl)methyl)-7-azaspiro[4.5]decane-7-carbonyl)-2-phenylpiperidin-4-yl)acetamide (10 mg, 0.016 mmol), potassium carbonate (10.8 mg, 0.078 mmol) in MeOH (1 mL) and water (0.1 mL) to give the title compound (4.2 mg, 47%) as a colourless solid. LCMS (Method B): R_{T}=0.93 min, m/z=543 [M+H]⁺. ¹H NMR (500 MHz, DMSO-d₆): δ 7.77-7.69 (m, 3H), 7.51-7.44 (m, 3H), 7.27-7.14 (m, 5H), 6.66 (d, J=2.1 Hz, 1H), 6.58 (dd, J=7.1, 2.1 Hz, 1H), 4.55-4.39 (m, 1H), 4.21 (dd, J=11.6, 3.5 Hz, 1H), 4.09 (dd, J=12.8, 3.1 Hz, 1H), 3.83 (dd, J=11.0, 6.9 Hz, 1H), 3.69 (t, J=11.7 Hz, 1H), 3.49 (ddd, J=14.0, 12.1, 4.1 Hz, 1H), 3.36 (d, J=12.9 Hz, 1H), 2.92 (ddt, J=19.5, 15.1, 5.6 Hz, 2H), 2.82 (s, 1H), 2.69 (d, J=12.8 Hz, 1H), 1.95-1.74 (m, 3H), 1.69-1.57 (m, 2H), 1.57-1.43 (m, 3H), 1.43-1.28 (m, 3H), 1.28-1.13 (m, 4H).

Example 86 was prepared similarly to Example 66 except using Intermediate 22 instead of Intermediate 11. Example 87 was prepared similarly to Example 65 except using Intermediate 22 instead of Intermediate 11. Example 88 and Example 89 were prepared similarly to Example 59 except using Intermediate 11 and Intermediate 22, respectively, and using General Procedure 6 instead of General Procedure 1 in both cases. Example 90 was prepared similarly to Example 70 except using Intermediate 24 instead of Intermediate 15. Example 91 was prepared similarly to Example 64 except using Intermediate 19 instead of Intermediate 10. Example 92 was prepared similarly to Example 77 except using Intermediate 25 instead of Intermediate 18.

### Example 93: N-((2S,4R)-1-((R)-10-((2-Oxo-4-phenylpyridin-1(2H)-yl)methyl)-7-azaspiro[4.5]decane-7-carbonyl)-2-phenylpiperidin-4-yl)acetamide

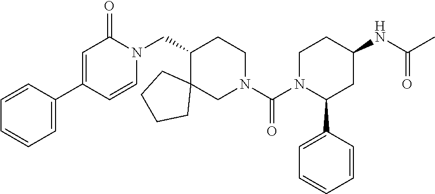 [SMILES: CC(=O)N[C@@H]1CCN(C(=O)N2CC[C@@H](Cn3ccc(-c4ccccc4)cc3=O)C3(CCCC3)C2)[C@H](c2ccccc2)C1]

To a stirred solution of 1-(((R)-7-((2S,4R)-4-amino-2-phenylpiperidine-1-carbonyl)-7-azaspiro[4.5]decan-10-yl)methyl)-4-phenylpyridin-2(1H)-one (40 mg, 0.075 mmol) [Example 23] and acetic anhydride (36 μL, 0.376 mmol) in DCM (0.5 mL) was added DIPEA (131 μL, 0.751 mmol). The reaction was stirred at rt for 1 h. The reaction was quenched with saturated NaHCO₃ (aq) solution and extracted with DCM (×3) using a phase separator. The combined organic phase was concentrated in vacuo and the crude material was purified by flash chromatography (10-100% EtOAc in cyclohexane; then 0-20% MeOH in EtOAc) to give the title compound (33 mg, 76%). LCMS (Method B): R_{T}=1.26 min, m/z=567 [M+H]⁺. ¹H NMR (500 MHz, DMSO-d₆): δ 7.78-7.68 (m, 4H), 7.53-7.43 (m, 3H), 7.26-7.12 (m, 5H), 6.66 (d, J=2.1 Hz, 1H), 6.58 (dd, J=7.1, 2.1 Hz, 1H), 4.08 (dd, J=12.7, 3.1 Hz, 1H), 3.98 (dd, J=11.6, 3.2 Hz, 1H), 3.86 (d, J=13.2 Hz, 1H), 3.70 (tdd, J=11.6, 7.2, 3.5 Hz, 2H), 3.41 (d, J=12.9 Hz, 1H), 3.29-3.20 (m, 1H), 2.88 (s, 1H), 2.70 (dd, J=15.6, 12.2 Hz, 2H), 1.93-1.71 (m, 7H), 1.71-1.43 (m, 5H), 1.43-1.26 (m, 4H), 1.18 (dq, J=14.1, 7.4 Hz, 2H).

Example 94 was prepared similarly to Example 78 except using Intermediate 26 instead of Intermediate 19. Example 95 was prepared similarly to Example 91 except using Intermediate 26 instead of Intermediate 19.

### Example 96: N-((2S,4R)-1-((R)-10-((2-Oxo-4-phenylpyridin-1(2H)-yl)methyl)-7-azaspiro[4.5]decane-7-carbonyl)-2-phenylpiperidin-4-yl)cyclopropanecarboxamide

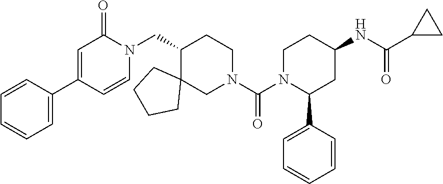 [SMILES: O=C(N[C@@H]1CCN(C(=O)N2CC[C@@H](Cn3ccc(-c4ccccc4)cc3=O)C3(CCCC3)C2)[C@H](c2ccccc2)C1)C1CC1]

Prepared according to General Procedure 3 using 1-(((R)-7-((2S,4R)-4-amino-2-phenylpiperidine-1-carbonyl)-7-azaspiro[4.5]decan-10-yl)methyl)-4-phenylpyridin-2(1H)-one (40 mg, 0.075 mmol) [Example 23], cyclopropanecarboxylic acid (7.8 mg, 0.090 mmol), HATU (34 mg, 0.090 mmol) and DIPEA (39 μL, 0.225 mmol) in DCM (1 mL) to give the title compound (27 mg, 60%). LCMS (Method B): R_{T}=1.37 min, m/z=593 [M+H]⁺. ¹H NMR (500 MHz, DMSO-d₆): δ 7.97 (d, J=7.6 Hz, 1H), 7.76-7.69 (m, 3H), 7.52-7.42 (m, 3H), 7.26-7.12 (m, 5H), 6.66 (d, J=2.1 Hz, 1H), 6.58 (dd, J=7.1, 2.1 Hz, 1H), 4.09 (dd, J=12.8, 3.1 Hz, 1H), 3.97 (dd, J=11.7, 3.2 Hz, 1H), 3.86 (dd, J=11.3, 6.6 Hz, 1H), 3.73 (dtd, J=12.2, 8.4, 8.0, 4.5 Hz, 2H), 3.42 (d, J=12.9 Hz, 1H), 3.26 (dt, J=12.3, 3.8 Hz, 1H), 2.88 (s, 1H), 2.70 (dd, J=12.6, 10.0 Hz, 2H), 1.95-1.72 (m, 4H), 1.72-1.13 (m, 12H), 0.68-0.54 (m, 4H).

Example 97 was prepared similarly to Example 92 except using Intermediate 6 and General Procedure 6 instead of Intermediate 1 and General Procedure 1. Example 98 was prepared similarly to Example 47 except using Intermediate 19 instead of Intermediate 6. Example 99 was prepared similarly to Example 97 except using Intermediate 19 instead of Intermediate 6. Example 100 was prepared similarly to Example 92 except using Intermediate 27 instead of Intermediate 25. Example 101 was prepared similarly to Example 97 except using Intermediate 27 instead of Intermediate 25. Example 102 was prepared similarly to Example 99 except using Intermediate 10 instead of Intermediate 19. Example 103 was prepared similarly to Example 99 except using Intermediate 28 instead of Intermediate 19. Example 104 was prepared similarly to Example 99 except using Intermediate 29 instead of Intermediate 19.

### Example 105: Methyl ((2S,4R)-1-((R)-10-((6-oxo-4-phenylpyrimidin-1(6H)-yl)methyl)-7-azaspiro[4.5]decane-7-carbonyl)-2-phenylpiperidin-4-yl)glycinate

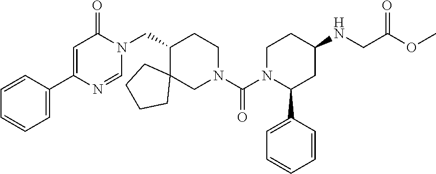 [SMILES: COC(=O)CN[C@@H]1CCN(C(=O)N2CC[C@@H](Cn3cnc(-c4ccccc4)cc3=O)C3(CCCC3)C2)[C@H](c2ccccc2)C1]

To a stirred solution of 3-(((R)-7-((2S,4R)-4-amino-2-phenylpiperidine-1-carbonyl)-7-azaspiro[4.5]decan-10-yl)methyl)-6-phenylpyrimidin-4(3H)-one (40 mg, 0.076 mmol) [Example 25] and potassium carbonate (31.5 mg, 0.228 mmol) in DMF (1 mL) was added methyl 2-bromoacetate (10 μL, 0.106 mmol). The reaction was stirred at rt for 20 h. The reaction was diluted with EtOAc and washed with water (×3), then with brine, dried (MgSO₄), filtered, and concentrated in vacuo. The resulting crude material was purified by flash chromatography using a KP-NH column (10-100% EtOAc in cyclohexane; then 0-20% MeOH in EtOAc) to give the title compound (36 mg, 74%). LCMS (Method B): R_{T}=0.98 min, m/z=598 [M+H]⁺. ¹H NMR (500 MHz, DMSO-d₆): δ 8.55 (s, 1H), 8.09-8.04 (m, 2H), 7.49 (dd, J=5.1, 1.9 Hz, 3H), 7.25-7.17 (m, 4H), 7.13 (ddt, J=8.6, 6.1, 1.8 Hz, 1H), 6.97 (d, J=0.8 Hz, 1H), 4.10 (dd, J=13.2, 3.1 Hz, 1H), 3.93 (dd, J=11.6, 3.1 Hz, 1H), 3.87 (d, J=13.3 Hz, 1H), 3.70 (t, J=12.0 Hz, 1H), 3.61 (s, 3H), 3.43 (d, J=12.7 Hz, 1H), 3.37-3.32 (m, 2H), 3.27-3.19 (m, 1H), 2.83 (s, 1H), 2.69-2.56 (m, 2H, obscured by DMSO satellite), 1.98 (s, 1H), 1.85 (dd, J=20.6, 10.6 Hz, 3H), 1.76 (s, 1H), 1.71-1.42 (m, 4H), 1.42-1.13 (m, 8H).

### Example 106: Sodium ((2S,4R)-1-((R)-10-((6-Oxo-4-phenylpyrimidin-1(6H)-yl)methyl)-7-azaspiro[4.5]decane-7-carbonyl)-2-phenylpiperidin-4-yl)glycinate

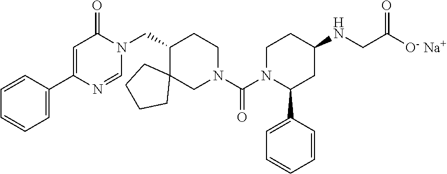

To a stirred solution of methyl ((2S,4R)-1-((R)-10-((6-oxo-4-phenylpyrimidin-1(6H)-yl)methyl)-7-azaspiro[4.5]decane-7-carbonyl)-2-phenylpiperidin-4-yl)glycinate (30 mg, 0.050 mmol) [Example 105] in MeOH (0.5 mL) was added 1 M NaOH_{(aq)} (55 μL, 0.055 mmol) and the reaction was stirred at rt for 96 h. The reaction was concentrated in vacuo and the resulting residue triturated with EtOAc (×3). The remaining solvents were removed in vacuo and the residual solid was lyophilised from solution in water/MeCN to give the title compound (27 mg, 79%). LCMS (Method B): R_{T}=1.08 min, m/z=584 [M+H]⁺. ¹H NMR (500 MHz, MeOH-d₄): δ 8.44 (s, 1H), 8.04-7.98 (m, 2H), 7.51-7.46 (m, 3H), 7.29-7.22 (m, 4H), 7.19-7.14 (m, 1H), 6.91 (s, 1H), 4.26 (dd, J=13.2, 3.1 Hz, 1H), 4.07-3.98 (m, 2H), 3.81 (t, J=11.9 Hz, 1H), 3.53 (d, J=13.0 Hz, 1H), 3.46-3.40 (m, 1H), 3.27-3.24 (m, 2H), 3.04 (s, 1H), 2.91-2.76 (m, 3H), 2.07 (t, J=14.0 Hz, 2H), 2.00-1.93 (m, 1H), 1.85 (s, 1H), 1.79-1.22 (m, 12H).

Example 107 was prepared similarly to Example 66 except using Intermediate 30 instead of Intermediate 11. Example 108 was prepared similarly to Example 65 except using Intermediate 30 instead of Intermediate 11. Example 109 was prepared similarly to Example 97 except using Intermediate 31 instead of Intermediate 25. Example 110 was prepared similarly to Example 97 except using Intermediate 32 instead of Intermediate 6 and no final deprotection step was required. Example 111 was prepared similarly to Example 64 except using Intermediate 33 instead of Intermediate 10 and no final deprotection step was required. Example 112 was prepared similarly to Example 64 except using Intermediate 34 instead of Intermediate 10 and no final deprotection step was required. Example 113 was prepared similarly to Example 53 except using Intermediate 19 instead of Intermediate 6. Example 114 was prepared similarly to Example 84 except using Intermediate 19 instead of Intermediate 6. Example 115 was prepared similarly to Example 92 except using tert-butyl (R)-3-(3-fluorophenyl)piperazine-1-carboxylate [prepared according to WO2019150119] instead of Intermediate 1. Example 116 was prepared similarly to Example 92 except using Intermediate 3 instead of Intermediate 1. Example 117 was prepared similarly to Example 99 except using Intermediate 26 instead of Intermediate 19. Example 118 was prepared similarly to Example 53 except using Intermediate 28 instead of Intermediate 6. Example 119 was prepared similarly to Example 114 except using Intermediate 10 instead of Intermediate 19. Example 120 was prepared similarly to Example 53 except using Intermediate 29 instead of Intermediate 6. Example 121 was prepared similarly to Example 91 except using Intermediate 35 instead of Intermediate 19. Example 122 was prepared similarly to Example 78 except using Intermediate 35 instead of Intermediate 19. Example 123 was prepared similarly to Example 83 except using Intermediate 19 instead of Intermediate 6. Example 124 was prepared similarly to Example 97 except using Intermediate 35 instead of Intermediate 6. Example 125 was prepared similarly to Example 101 except using Intermediate 19 instead of Intermediate 6. Example 126 was prepared similarly to Example 53 except using Intermediate 35 instead of Intermediate 6. Example 127 was prepared similarly to Example 117 except using Intermediate 36 instead of Intermediate 25. Example 128 was prepared similarly to Example 117 except using Intermediate 37 instead of Intermediate 25. Example 129 was prepared similarly to Example 117 except using Intermediate 38 instead of Intermediate 25. Example 130 was prepared similarly to Example 117 except using Intermediate 39 instead of Intermediate 25. Example 131 was prepared similarly to Example 53 except using Intermediate 40 instead of Intermediate 6. Example 132 was prepared similarly to Example 56 except using Intermediate 40 instead of Intermediate 6. Example 133 was prepared similarly to Example 56 except using Intermediate 41 instead of Intermediate 6. Example 134 was prepared similarly to Example 97 except using Intermediate 42 instead of Intermediate 6. Example 135 was prepared similarly to Example 56 except using Intermediate 43 instead of Intermediate 6. Example 136 was prepared similarly to Example 84 except using Intermediate 40 instead of Intermediate 6. Example 137 was prepared similarly to Example 84 except using Intermediate 26 instead of Intermediate 6. Example 138 was prepared similarly to Example 97 except using Intermediate 41 instead of Intermediate 6. Example 139 was prepared similarly to Example 84 except using Intermediate 41 instead of Intermediate 6. Example 140 was prepared similarly to Example 109 except using Intermediate 40 instead of Intermediate 6. Example 141 was prepared similarly to Example 109 except using Intermediate 26 instead of Intermediate 6. Example 142 was prepared similarly to Example 109 except using Intermediate 41 instead of Intermediate 6. Example 143 was prepared similarly to Example 109 except using Intermediate 35 instead of Intermediate 6. Example 144 was prepared similarly to Example 56 except using Intermediate 44 instead of Intermediate 6. Example 145 was prepared similarly to Example 109 except using Intermediate 19 instead of Intermediate 6. Example 146 was prepared similarly to Example 84 except using Intermediate 35 instead of Intermediate 6. Example 147 was prepared similarly to Example 56 except using Intermediate 45 instead of Intermediate 6. Example 148 was prepared similarly to Example 56 except using Intermediate 46 instead of Intermediate 6. Example 149 was prepared similarly to Example 97 except using Intermediate 40 instead of Intermediate 6. Example 150 was prepared similarly to Example 56 except using Intermediate 47 instead of Intermediate 6. Example 151 was prepared similarly to Example 56 except using Intermediate 48 instead of Intermediate 6. Example 152 was prepared similarly to Example 130 except using Intermediate 42 instead of Intermediate 26. Example 153 was prepared similarly to Example 101 except using Intermediate 35 instead of Intermediate 6.

### Example 154: tert-Butyl ((2S,4R)-2-(2,5-difluorophenyl)-1-((R)-10-((6-oxo-4-phenylpyrimidin-1(6H)-yl)methyl)-7-azaspiro[4.5]decane-7-carbonyl)piperidin-4-yl)glycinate

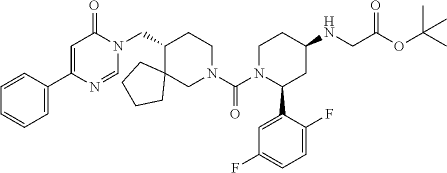 [SMILES: CC(C)(C)OC(=O)CN[C@@H]1CCN(C(=O)N2CC[C@@H](Cn3cnc(-c4ccccc4)cc3=O)C3(CCCC3)C2)[C@H](c2cc(F)ccc2F)C1]

To a stirred solution of 3-(((R)-7-((2S,4R)-4-amino-2-(2,5-difluorophenyl)piperidine-1-carbonyl)-7-azaspiro[4.5]decan-10-yl)methyl)-6-phenylpyrimidin-4(3H)-one (72 mg, 0.128 mmol) [Example 86] and potassium carbonate (53 mg, 0.385 mmol) in DMF (1 mL) was added tert-butyl 2-bromoacetate (19 μL, 0.128 mmol) at rt. After 1 h, the reaction was diluted with DCM and washed with water (×3), then brine, dried (MgSO₄), filtered, and concentrated in vacuo. The resulting crude material was purified by flash chromatography using a Biotage Sfar Amino D column (10-100% EtOAc in cyclohexane) to give the title compound (48 mg, 53%). LCMS (Method B): R_{T}=1.06 min, m/z=676 [M+H]⁺. ¹H NMR (500 MHz, DMSO-d₆): δ 9.08 (s, 2H), 8.54 (s, 1H), 8.06 (dd, J=6.6, 3.0 Hz, 2H), 7.49 (dd, J=5.2, 2.0 Hz, 3H), 7.19 (td, J=9.3, 4.5 Hz, 1H), 7.10 (ddd, J=8.6, 6.5, 3.6 Hz, 1H), 7.01-6.93 (m, 2H), 4.33-4.23 (m, 1H), 4.10 (dd, J=12.9, 3.0 Hz, 1H), 4.01-3.80 (m, 3H), 3.73 (s, 1H), 3.46-3.34 (m, 2H), 2.85 (s, 1H), 2.77 (d, J=12.9 Hz, 1H), 2.69 (t, J=12.2 Hz, 1H), 2.11 (s, 2H), 1.94-1.84 (m, 1H), 1.84-1.48 (m, 7H), 1.46 (s, 9H), 1.41-1.13 (m, 5H).

### Example 155: N-((2S,4R)-2-(2,5-Difluorophenyl)-1-((R)-10-((6-oxo-4-phenylpyrimidin-1(6H)-yl)methyl)-7-azaspiro[4.5]decane-7-carbonyl)piperidin-4-yl)acetamide

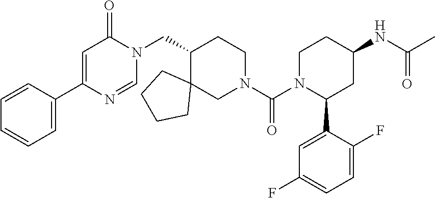 [SMILES: CC(=O)N[C@@H]1CCN(C(=O)N2CC[C@@H](Cn3cnc(-c4ccccc4)cc3=O)C3(CCCC3)C2)[C@H](c2cc(F)ccc2F)C1]

To a stirred solution of 3-(((R)-7-((2S,4R)-4-amino-2-(2,5-difluorophenyl)piperidine-1-carbonyl)-7-azaspiro[4.5]decan-10-yl)methyl)-6-phenylpyrimidin-4(3H)-one (26 mg, 0.046 mmol) [Example 86] and acetic anhydride (22 μL, 0.232 mmol) in DCM (0.5 mL) was added DIPEA (81 μL, 0.463 mmol) at rt. After 1.5 h, the reaction was quenched with saturated NaHCO_{3(aq)} and extracted with DCM (×3) using a phase separator. The combined organic phase was concentrated in vacuo and the crude material was purified by flash chromatography (10-100% EtOAc in cyclohexane; then 0-20% MeOH in EtOAc) to give the title compound (19 mg, 68%). LCMS (Method B): R_{T}=1.29 min. m/z=604 [M+H]⁺. ¹H NMR (500 MHz, DMSO-d₆): δ 8.55 (s, 1H), 8.06 (dd, J=6.6, 3.0 Hz, 2H), 7.80 (d, J=7.5 Hz, 1H), 7.49 (dd, J=5.1, 1.9 Hz, 3H), 7.15 (td, J=9.3, 4.5 Hz, 1H), 7.04 (tt, J=8.2, 3.4 Hz, 1H), 6.99-6.92 (m, 2H), 4.30-4.24 (m, 1H), 4.11 (dd, J=13.1, 3.0 Hz, 1H), 3.90-3.81 (m, 1H), 3.70 (dtd, J=11.4, 7.2, 3.5 Hz, 2H), 3.40 (d, J=12.9 Hz, 1H), 3.28 (t, J=3.5 Hz, 1H), 2.88 (s, 1H), 2.79-2.67 (m, 2H), 1.88 (q, J=5.1 Hz, 3H), 1.83-1.72 (m, 4H), 1.65 (d, J=18.9 Hz, 2H), 1.60-1.45 (m, 3H), 1.43-1.25 (m, 4H), 1.21 (dt, J=14.1, 6.9 Hz, 2H).

Example 156 was prepared similarly to Example 56 except using Intermediate 49 instead of Intermediate 6. Example 157 was prepared similarly to Example 101 except using Intermediate 42 instead of Intermediate 6.

### Example 158: ((2S,4R)-2-(2,5-Difluorophenyl)-1-((R)-10-((6-oxo-4-phenylpyrimidin-1(6H-yl)methyl)-7-azaspiro[4.5]decane-7-carbonyl)piperidin-4-yl)glycine

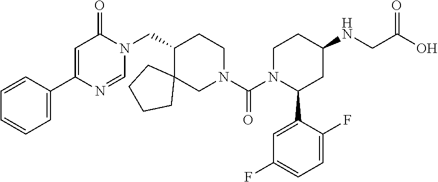 [SMILES: O=C(O)CN[C@@H]1CCN(C(=O)N2CC[C@@H](Cn3cnc(-c4ccccc4)cc3=O)C3(CCCC3)C2)[C@H](c2cc(F)ccc2F)C1]

A solution of tert-butyl ((2S,4R)-2-(2,5-difluorophenyl)-1-((R)-10-((6-oxo-4-phenylpyrimidin-1(6H)-yl)methyl)-7-azaspiro[4.5]decane-7-carbonyl)piperidin-4-yl)glycinate (35 mg, 0.052 mmol) [Example 154] in 4 M HCl in 1,4-dioxane (1 mL) was stirred at rt for 18 h. The reaction mixture was concentrated in vacuo and purified by reversed phase preparative HPLC to give the title compound (22 mg, 68%). LCMS (Method B): R_{T}=1.02 min, m/z=620 [M+H]⁺. ¹H NMR (500 MHz, DMSO-d₆): δ 8.54 (s, 1H), 8.06 (dd, J=6.7, 3.0 Hz, 2H), 7.49 (dd, J=5.1, 1.9 Hz, 3H), 7.16 (td, J=9.3, 4.5 Hz, 1H), 7.06 (ddd, J=8.9, 7.4, 3.5 Hz, 1H), 7.00-6.93 (m, 2H), 4.29-4.21 (m, 1H), 4.11 (dd, J=13.1, 3.0 Hz, 1H), 3.84 (dt, J=12.5, 3.8 Hz, 1H), 3.70 (d, J=11.1 Hz, 1H), 3.46-3.36 (m, 3H, obscured by HDO signal), 3.18-3.09 (m, 3H), 3.02 (d, J=11.5 Hz, 1H), 2.85 (s, 1H), 2.75 (d, J=12.9 Hz, 1H), 2.70-2.64 (m, 1H), 2.01 (d, J=10.9 Hz, 2H), 1.86 (td, J=10.6, 5.3 Hz, 1H), 1.75 (s, 1H), 1.72-1.43 (m, 6H), 1.42-1.12 (m, 5H).

### Example 159: 4-(((R)-7-((2S,4R)-2-(2,5-Difluorophenyl)-4-(methylamino)piperidine-1-carbonyl)-7-azaspiro[4.5]decan-10-ylmethyl)morpholin-3-one

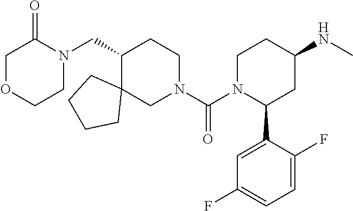 [SMILES: CN[C@@H]1CCN(C(=O)N2CC[C@@H](CN3CCOCC3=O)C3(CCCC3)C2)[C@H](c2cc(F)ccc2F)C1]

Step 1: N-((2S,4R)-2-(2,5-difluorophenyl)-1-(10-((3-oxomorpholino)methyl)-7-azaspiro[4.5]decane-7-carbonyl)piperidin-4-yl)-2,2,2-trifluoro-N-methylacetamide: The title compound was prepared similarly to Example 41 except using Intermediate 19 and General Procedure 6 instead of Intermediate 1 and General Procedure 1. LCMS (Method A): R_{T}=1.36 min, m/z=601 [M+H]⁺.

Step 2: N-((2S,4R)-2-(2,5-Difluorophenyl)-1-((R)-10-((3-oxomorpholino)methyl)-7-azaspiro[4.5]decane-7-carbonyl)piperidin-4-yl)-2,2,2-trifluoro-N-methylacetamide: N-((2S,4R)-2-(2,5-difluorophenyl)-1-(10-((3-oxomorpholino)methyl)-7-azaspiro[4.5]decane-7-carbonyl)piperidin-4-yl)-2,2,2-trifluoro-N-methylacetamide (210 mg) was separated into the single stereoisomers by chiral HPLC using a Lux C4 (21.2 mm×250 mm, 5 μm) column with isocratic solvent conditions: 100% MeOH (Flow rate: 21 mL/min) to give the title compound (first eluting isomer, 1.25 min: 73.9 mg); and N-((2S,4R)-2-(2,5-difluorophenyl)-1-((S)-10-((3-oxomorpholino)methyl)-7-azaspiro[4.5]decane-7-carbonyl)piperidin-4-yl)-2,2,2-trifluoro-N-methylacetamide (second eluting isomer, 2.78 min: 75.0 mg). [Note: stereochemistry was assigned by comparison of the potency SAR of the deprotected derivatives with close analogues].

Step 3: 4-(((R)-7-((2S,4R)-2-(2,5-Difluorophenyl)-4-(methylamino)piperidine-1-carbonyl)-7-azaspiro[4.5]decan-10-yl)methyl)morpholin-3-one: Prepared according to General Procedure 6 using N-((2S,4R)-2-(2,5-difluorophenyl)-1-((R)-10-((3-oxomorpholino)methyl)-7-azaspiro[4.5]decane-7-carbonyl)piperidin-4-yl)-2,2,2-trifluoro-N-methylacetamide (70 mg, 0.117 mmol) and potassium carbonate (80.5 mg, 0.583 mmol) in a solution of MeOH (1 mL) and water (0.1 mL). The crude residue was purified by flash chromatography using a Biotage Sfar Amino D column (10-100% EtOAc in cyclohexane; then 0-10% MeOH in EtOAc) and freeze-dried to give the title compound (29.1 mg, 49%). LCMS (Method B): R_{T}=0.72 min, m/z=505 [M+H]⁺. ¹H NMR (500 MHz, DMSO-d₆): δ 7.13 (td, J=9.2, 4.5 Hz, 1H), 7.00 (dddd, J=14.9, 8.9, 6.5, 3.4 Hz, 2H), 4.24 (dd, J=11.8, 2.5 Hz, 1H), 4.02 (s, 2H), 3.85-3.73 (m, 3H), 3.67 (s, 1H), 3.37 (dt, J=12.3, 5.1 Hz, 1H), 3.29-3.17 (m, 3H), 3.16-2.96 (m, 2H), 2.81 (d, J=12.8 Hz, 1H), 2.69-2.60 (m, 1H), 2.45-2.38 (m, 1H), 2.24 (s, 3H), 1.98-1.87 (m, 2H), 1.80-1.69 (m, 1H), 1.67-1.29 (m, 8H), 1.29-1.08 (m, 5H).

Example 160 was prepared similarly to Example 56 except using Intermediate 50 instead of Intermediate 6.

### Example 161: 3-(((R)-7-((2S,4R)-4-((2-Hydroxyethyl)amino)-2-phenylpiperidine-1-carbonyl)-7-azaspiro[4.5]decan-10-yl)methyl)-6-phenylpyrimidin-4(3H)-one

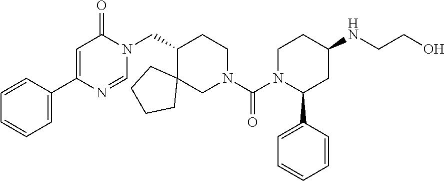 [SMILES: O=C(N1CC[C@@H](Cn2cnc(-c3ccccc3)cc2=O)C2(CCCC2)C1)N1CC[C@@H](NCCO)C[C@H]1c1ccccc1]

Step 1: (R)-10-((6-Oxo-4-phenylpyrimidin-1(6H)-yl)methyl)-7-azaspiro[4.5]decane-7-carbonyl chloride: Prepared according to General Procedure 8 using (R)-3-((7-azaspiro[4.5]decan-10-yl)methyl)-6-phenylpyrimidin-4(3H)-one (330 mg, 1.02 mmol), triphosgene (242 mg, 0.816 mmol) and pyridine (0.83 mL) in DCM (30 mL) to give the title compound (400 mg, quantitative). LCMS (Method B): R_{T}=1.43 min, m/z=386 [M+H]⁺.

Step 2: N-(2-((tert-Butyldimethylsilyl)oxy)ethyl)-2,2,2-trifluoro-N-((2S,4R)-1-((R)-10-((6-oxo-4-phenylpyrimidin-1(6H)-yl)methyl)-7-azaspiro[4.5]decane-7-carbonyl)-2-phenylpiperidin-4-yl)acetamide: Prepared according to General Procedure 7 using (R)-10-((6-oxo-4-phenylpyrimidin-1(6H)-yl)methyl)-7-azaspiro[4.5]decane-7-carbonyl chloride (50 mg, 0.130 mmol), N-(2-((tert-butyldimethylsilyl)oxy)ethyl)-2,2,2-trifluoro-N-((2S,4R)-2-phenylpiperidin-4-yl)acetamide (61 mg, 0.143 mmol) [Intermediate 51] and DIPEA (121 μL, 0.694 mmol) in DCM (2 mL) to give the title compound (76 mg, 75%). LCMS (Method A): R_{T}=1.97 min, m/z=not observed since >750.

Step 3: 3-(((R)-7-((2S,4R)-4-((2-Hydroxyethyl)amino)-2-phenylpiperidine-1-carbonyl)-7-azaspiro[4.5]decan-10-yl)methyl)-6-phenylpyrimidin-4(3H)-one: Potassium carbonate (133 mg, 0.962 mmol) was added to a stirred solution of N-(2-((tert-butyldimethylsilyl)oxy)ethyl)-2,2,2-trifluoro-N-((2S,4R)-1-((R)-10-((6-oxo-4-phenylpyrimidin-1(6H)-yl)methyl)-7-azaspiro[4.5]decane-7-carbonyl)-2-phenylpiperidin-4-yl)acetamide (75 mg, 0.096 mmol) in MeOH (2 mL) and water (0.2 mL) and the reaction stirred at rt for 48 h. Water was added to the reaction and the resulting mixture was extracted with DCM (×3) using a phase separator. The combined organic phase was concentrated in vacuo and the resulting residue was taken up in DCM (3 mL). TFA (1 mL) was added and the reaction stirred for 18 h. The desired product was isolated using a Biotage SCX-2 cartridge (pre-equilibrated with 3:1 DCM/MeOH, reaction mixture loaded and cartridge washed with 3:1 DCM/MeOH, product eluted using 3:1 DCM/7 M ammonia in MeOH) and further purified by flash chromatography using a Biotage Sfar Amino D column (10-100% EtOAc in cyclohexane; then 0-10% MeOH in EtOAc) to give the title compound (31.6 mg, 57%). LCMS (Method B): R_{T}=0.90 min, m/z=570 [M+H]⁺. ¹H NMR (500 MHz, DMSO-d₆): δ 8.55 (s, 1H), 8.06 (dd, J=6.6, 3.0 Hz, 2H), 7.49 (dd, J=5.1, 1.9 Hz, 3H), 7.25-7.16 (m, 4H), 7.16-7.09 (m, 1H), 6.97 (s, 1H), 4.41 (t, J=5.3 Hz, 1H), 4.10 (dd, J=13.0, 3.0 Hz, 1H), 3.95 (dd, J=11.7, 3.1 Hz, 1H), 3.92-3.83 (m, 1H), 3.70 (t, J=12.0 Hz, 1H), 3.44 (d, J=13.0 Hz, 1H), 3.40 (q, J=5.6 Hz, 2H), 3.25 (dt, J=12.2, 3.7 Hz, 1H), 2.83 (s, 1H), 2.72-2.62 (m, 2H), 2.58 (t, J=5.9 Hz, 3H), 1.95-1.81 (m, 3H), 1.77 (s, 1H), 1.72-1.42 (m, 4H), 1.42-1.12 (m, 7H). [OH signal not observed].

Example 162 was prepared similarly to Example 101 except using Intermediate 49 instead of Intermediate 6. Example 163 was prepared similarly to Example 78 except using Intermediate 52 instead of tert-butyl (R)-10-((6-oxo-4-phenylpyrimidin-1(6H)-yl)methyl)-7-azaspiro[4.5]decane-7-carboxylate. Example 164 was prepared similarly to Example 96 except using 3-(((R)-7-((2S,4R)-4-amino-2-(2,5-difluorophenyl)piperidine-1-carbonyl)-7-azaspiro[4.5]decan-10-yl)methyl)-6-phenylpyrimidin-4(3H)-one [Example 86] instead of 1-(((R)-7-((2S,4R)-4-amino-2-phenylpiperidine-1-carbonyl)-7-azaspiro[4.5]decan-10-yl)methyl)-4-phenylpyridin-2(1H)-one. Example 165 was prepared similarly to Example 164 except using picolinic acid instead of cyclopropanecarboxylic acid. Example 166 was prepared similarly to Example 101 except using Intermediate 40 instead of Intermediate 6. Example 167 was prepared similarly to Example 101 except using Intermediate 10 instead of Intermediate 6.

### Example 168: 1-(((R)-7-((2S,4R)-2-(2,5-Difluorophenyl)-4-(methylamino)piperidine-1-carbonyl)-7-azaspiro[4.5]decan-10-yl)methyl)-4-phenyl-5,6-dihydropyridin-2(1H)-one

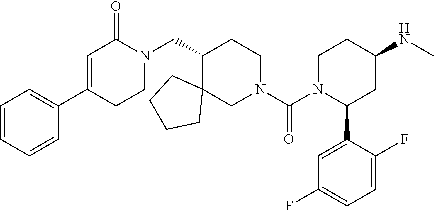 [SMILES: CN[C@@H]1CCN(C(=O)N2CC[C@@H](CN3CCC(c4ccccc4)=CC3=O)C3(CCCC3)C2)[C@H](c2cc(F)ccc2F)C1]

Step 1: 1-((7-Azaspiro[4.5]decan-10-yl)methyl)-4-phenyl-5,6-dihydropyridin-2(1H)-one: Prepared according to General Procedure 1 using tert-butyl 10-((6-oxo-4-phenyl-3,6-dihydropyridin-1(2H)-yl)methyl)-7-azaspiro[4.5]decane-7-carboxylate (150.0 mg, 0.35 mmol) and 3:2 TFA/DCM (2.5 mL) and stirred for 20 min to give the title compound (121 mg, 99%). LCMS (Method B): R_{T}=0.71 min, m/z 325 [M+H]⁺.

Step 2: Trifluoromethyl ((2S,4R)-1-(chlorocarbonyl)-2-(2,5-difluorophenyl)piperidin-4-yl)(methyl)carbamate: Prepared according to General Procedure 8 using triphosgene (49.6 mg, 0.17 mmol), pyridine (135 uL, 1.7 mmol), N-((2S,4R)-2-(2,5-difluorophenyl)piperidin-4-yl)-2,2,2-trifluoro-N-methylacetamide hydrochloride (120 mg, 0.33 mmol) and DIPEA (87.6 uL, 0.50 mmol) to give the title compound (119 mg, 92%).

Step 3: N-((2S,4R)-2-(2,5-Difluorophenyl)-1-(10-((6-oxo-4-phenyl-3,6-dihydropyridin-1(2H)-yl)methyl)-7-azaspiro[4.5]decane-7-carbonyl)piperidin-4-yl)-2,2,2-trifluoro-N-methylacetamide: Prepared according to General Procedure 7 using 1-((7-azaspiro[4.5]decan-10-yl)methyl)-4-phenyl-5,6-dihydropyridin-2(1H-one (121 mg, 0.35 mmol), DIPEA (307 uL, 1.76 mmol) and trifluoromethyl ((2S,4R)-1-(chlorocarbonyl)-2-(2,5-difluorophenyl)piperidin-4-yl)(methyl)carbamate (119 mg, 0.31 mmol) to give the title compound (159 mg, 67%). LCMS (Method B): R_{T}=1.66 min, m/z 673 [M+H]⁺.

Step 4: N-((2S,4R)-2-(2,5-Difluorophenyl)-1-((R)-10-((6-oxo-4-phenyl-3,6-dihydropyridin-1(2H)-yl)methyl)-7-azaspiro[4.5]decane-7-carbonyl)piperidin-4-yl)-2,2,2-trifluoro-N-methylacetamide: N-((2S,4R)-2-(2,5-Difluorophenyl)-1-(10-((6-oxo-4-phenyl-3,6-dihydropyridin-1 (2H)-yl)methyl)-7-azaspiro[4.5]decane-7-carbonyl)piperidin-4-yl)-2,2,2-trifluoro-N-methylacetamide (157 mg) was separated into the single stereoisomers by chiral preparative SFC using a Lux C4 (21.2 mm×250 mm, 5 μm) column with isocratic solvent conditions: 50:50 MeOH/CO₂ (Flow rate: 50 mL/min) to give the title compound (first eluting isomer, 2.15 min: 53.7 mg); and N-((2S,4R)-2-(2,5-difluorophenyl)-1-((S)-10-((6-oxo-4-phenyl-3,6-dihydropyridin-1(2H)-yl)methyl)-7-azaspiro[4.5]decane-7-carbonyl)piperidin-4-yl)-2,2,2-trifluoro-N-methylacetamide (second eluting isomer, 3.76 min: 62.1 mg). [Note: stereochemistry was assigned by comparison of the potency SAR of the deprotected derivatives with close analogues].

Step 5: 1-(((R)-7-((2S,4R)-2-(2,5-Difluorophenyl)-4-(methylamino)piperidine-1-carbonyl)-7-azaspiro[4.5]decan-10-yl)methyl)-4-phenyl-5,6-dihydropyridin-2(1H)-one: Prepared according to General Procedure 6 using N-((2S,4R)-2-(2,5-difluorophenyl)-1-((R)-10-((6-oxo-4-phenyl-3,6-dihydropyridin-1(2H)-yl)methyl)-7-azaspiro[4.5]decane-7-carbonyl)piperidin-4-yl)-2,2,2-trifluoro-N-methylacetamide (53 mg, 0.080 mmol) and potassium carbonate (55 mg, 0.40 mmol) to give the title compound (45 mg, 98%). LCMS (Method B): R_{T}=0.99 min, m/z 577 [M+H]⁺. ¹H NMR (500 MHz, DMSO-d₆): δ 7.62 (d, 2H), 7.51-7.35 (m, 3H), 7.13 (td, 1H), 7.07-6.95 (m, 2H), 6.20 (s, 1H), 4.24 (d, 1H), 3.84-3.74 (m, 1H), 3.67-3.56 (m, 1H), 3.55-3.43 (m, 2H), 3.28-3.19 (m, 2H), 3.19-3.01 (m, 2H), 2.87-2.72 (m, 3H), 2.69-2.61 (m, 1H), 2.47-2.40 (m, 2H), 2.25 (s, 3H), 1.92 (d, 2H), 1.77-1.68 (m, 1H), 1.65-1.47 (m, 5H), 1.47-1.32 (m, 2H), 1.32-1.10 (m, 5H).

### Example 169: 3-(((R)-7-((2S,4R)-2-(2,5-Difluorophenyl)-4-((pyrimidin-2-ylmethyl)amino)piperidine-1-carbonyl)-7-azaspiro[4.5]decan-10-yl)methyl)-6-phenylpyrimidin-4(3H)-one

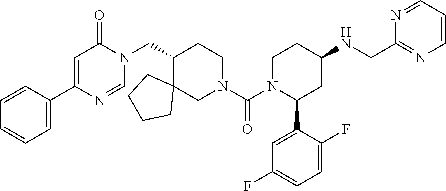 [SMILES: O=C(N1CC[C@@H](Cn2cnc(-c3ccccc3)cc2=O)C2(CCCC2)C1)N1CC[C@@H](NCc2ncccn2)C[C@H]1c1cc(F)ccc1F]

To a stirred solution of 3-(((R)-7-((2S,4R)-4-amino-2-(2,5-difluorophenyl)piperidine-1-carbonyl)-7-azaspiro[4.5]decan-10-yl)methyl)-6-phenylpyrimidin-4(3H)-one (40 mg, 0.071 mmol) [Example 86] and 2-(bromomethyl)pyrimidine (12 mg, 0.071 mmol) in DMF (1 mL) was added potassium carbonate (29 mg, 0.213 mmol). The reaction was stirred at rt for 1 h. The reaction was diluted with DCM and washed with water (×3), then with brine, filtered (phase separator), and concentrated in vacuo. The remaining residue was purified by flash chromatography using a Biotage Sfar Amino D column (10-100% EtOAc in cyclohexane; then 0-10% MeOH in EtOAc; followed by Silisep silica column, 0-20% MeOH in EtOAc) to give the title compound (16.4 mg, 34%). LCMS (Method B): R_{T}=0.94 min, m/z=654 [M+H]⁺. ¹H NMR (500 MHz, DMSO-d₆): δ 8.85 (d, J=4.9 Hz, 2H), 8.55 (s, 1H), 8.06 (dd, J=6.7, 3.0 Hz, 2H), 7.49 (dd, J=5.2, 2.0 Hz, 4H), 7.17 (td, J=9.3, 4.5 Hz, 1H), 7.07 (tt, J=8.3, 3.5 Hz, 1H), 7.03-6.95 (m, 2H), 4.30 (d, J=11.6 Hz, 3H), 4.11 (dd, J=13.1, 3.0 Hz, 1H), 3.85 (d, J=12.9 Hz, 1H), 3.73 (t, J=11.2 Hz, 1H), 3.45-3.37 (m, 1H), 3.37-3.31 (m, 1H), 2.87 (s, 1H), 2.77 (d, J=12.9 Hz, 1H), 2.69 (t, J=12.3 Hz, 1H), 2.46-2.41 (m, 1H, obscured by DMSO signal), 2.12 (s, 2H), 1.88 (t, J=10.7 Hz, 1H), 1.64 (q, J=43.9, 35.7 Hz, 7H), 1.42-1.13 (m, 6H).

### Example 170: 3-(((R)-7-((2S,4R)-2-(2,5-Difluorophenyl)-4-((pyrimidin-4-ylmethyl)amino)piperidine-1-carbonyl)-7-azaspiro[4.5]decan-10-yl)methyl)-6-phenylpyrimidin-4(3H)-one

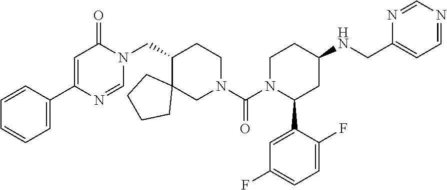 [SMILES: O=C(N1CC[C@@H](Cn2cnc(-c3ccccc3)cc2=O)C2(CCCC2)C1)N1CC[C@@H](NCc2ccncn2)C[C@H]1c1cc(F)ccc1F]

To a stirred solution of 3-(((R)-7-((2S,4R)-4-amino-2-(2,5-difluorophenyl)piperidine-1-carbonyl)-7-azaspiro[4.5]decan-10-yl)methyl)-6-phenylpyrimidin-4(3H)-one (40 mg, 0.071 mmol) [Example 86] and 4-(chloromethyl)pyrimidine (9.1 mg, 0.071 mmol) in DMF (1 mL) was added potassium carbonate (29 mg, 0.213 mmol). The reaction was stirred at rt for 65 h. The reaction was diluted with DCM and washed with water (×3), then with brine, filtered (phase separator), and concentrated in vacuo. The remaining residue was purified by flash chromatography using a Biotage Sfar Amino D column (10-100% EtOAc in cyclohexane; then 0-10% MeOH in EtOAc; followed by Silisep silica column, 0-20% MeOH in EtOAc) to give the title compound (19.6 mg, 41%). LCMS (Method B): R_{T}=0.94 min, m/z=654 [M+H]⁺. ¹H NMR (500 MHz, DMSO-d₆): δ 9.17 (s, 1H), 8.80 (s, 1H), 8.55 (s, 1H), 8.08-8.03 (m, 2H), 7.59 (dd, J=5.2, 1.4 Hz, 1H), 7.49 (dd, J=5.0, 1.9 Hz, 3H), 7.17 (td, J=9.3, 4.4 Hz, 1H), 7.07 (s, 1H), 7.02-6.95 (m, 2H), 4.38-3.96 (m, 3H), 3.85 (d, J=13.2 Hz, 1H), 3.73 (s, 1H), 3.39 (s, 1H), 3.34 (s, 1H), 2.86 (s, 1H), 2.77 (d, J=12.9 Hz, 1H), 2.74-2.65 (m, 1H), 2.53-2.51 (m, 2H), 2.09 (s, 2H), 1.92-1.84 (m, 1H), 1.78 (s, 1H), 1.73-1.43 (m, 6H), 1.43-1.13 (m, 6H).

Example 171 was prepared similarly to Example 101 except using Intermediate 32 instead of Intermediate 6 and no final deprotection step was required, however, 4 M HCl in dioxane was added to aid dissolution in acetonitrile/water before lyophilisation, leading to formation of the HCl salt.

### Example 172: N-((2S,4R)-2-(2,5-Difluorophenyl)-1-((R)-10-((6-fluoro-4-oxoquinazolin-3(4H)-yl)methyl)-7-azaspiro[4.5]decane-7-carbonyl)piperidin-4-yl)acetamide

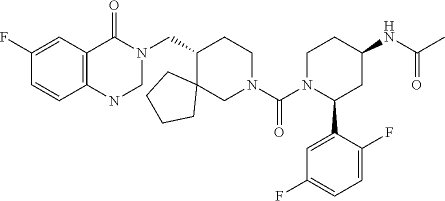 [SMILES: CC(=O)N[C@@H]1CCN(C(=O)N2CC[C@@H](CN3CNc4ccc(F)cc4C3=O)C3(CCCC3)C2)[C@H](c2cc(F)ccc2F)C1]

Step 1: (R)-10-((6-Fluoro-4-oxoquinazolin-3(4H)-yl)methyl)-7-azaspiro[4.5]decane-7-carbonyl chloride: Prepared according to General Procedure 8 using triphosgene (150 mg, 0.507 mmol), pyridine (0.51 mL, 6.34 mmol) and (R)-3-((7-azaspiro[4.5]decan-10-yl)methyl)-6-fluoroquinazolin-4(3H)-one (200 mg, 0.634 mmol) in DCM (10 mL) to give the title compound (220 mg, 91%). LCMS (Method B): R_{T}=1.42 min, m/z=378 [M+H]⁺.

Step 2: tert-Butyl ((2S,4R)-2-(2,5-difluorophenyl)-1-((R)-10-((6-fluoro-4-oxoquinazolin-3(4H)-yl)methyl)-7-azaspiro[4.5]decane-7-carbonyl)piperidin-4-yl)carbamate: Prepared according to General Procedure 7 using (R)-10-((6-fluoro-4-oxoquinazolin-3(4H)-yl)methyl)-7-azaspiro[4.5]decane-7-carbonyl chloride (150 mg, 0.397 mmol), tert-butyl ((2S,4R)-2-(2,5-difluorophenyl)piperidin-4-yl)carbamate (149 mg, 0.476 mmol) and DIPEA (0.35 mL, 1.99 mmol) in MeCN at 50° C. to give the title compound (220 mg, 85%). LCMS (Method B): R_{T}=1.61 min, m/z=598 [M−butene+H]⁺.

Step 3: 3-(((R)-7-((2S,4R)-4-Amino-2-(2,5-difluorophenyl)piperidine-1-carbonyl)-7-azaspiro[4.5]decan-10-yl)methyl)-6-fluoroquinazolin-4(3H)-one: Prepared according to General Procedure 1 using tert-butyl ((2S,4R)-2-(2,5-difluorophenyl)-1-((R)-10-((6-fluoro-4-oxoquinazolin-3(4H)-yl)methyl)-7-azaspiro[4.5]decane-7-carbonyl)piperidin-4-yl)carbamate (220 mg, 0.337 mmol) and TFA (2 mL) in DCM (4 mL) to give the title compound (170 mg, 91%). LCMS (Method B): R_{T}=0.91 min, m/z=554 [M+H]⁺.

Step 4: N-((2S,4R)-2-(2,5-Difluorophenyl)-1-((R)-10-((6-fluoro-4-oxoquinazolin-3(4H)-yl)methyl)-7-azaspiro[4.5]decane-7-carbonyl)piperidin-4-yl)acetamide: To a stirred solution of 3-(((R)-7-((2S,4R)-4-amino-2-(2,5-difluorophenyl)piperidine-1-carbonyl)-7-azaspiro[4.5]decan-10-yl)methyl)-6-fluoroquinazolin-4(3H)-one (40 mg, 0.072 mmol) and acetic anhydride (34 μL, 0.361 mmol) in DCM (1 mL) was added DIPEA (0.13 mL, 0.723 mmol). The reaction was stirred at rt for 18 h. The reaction mixture was then quenched with saturated NaHCO_{3(aq)} and extracted with DCM (×3) using a phase separator. The combined organic phase was concentrated in vacuo and the resulting residue was purified by flash chromatography (10-100% EtOAc in cyclohexane; then 0-10% MeOH in EtOAc) to give the title compound (25.8 mg, 60%). LCMS (Method B): R_{T}=1.26 min, m/z=596 [M+H]⁺. ¹H NMR (500 MHz, DMSO-d₆): δ 8.37 (s, 1H), 7.86-7.70 (m, 4H), 7.15 (td, J=9.3, 4.5 Hz, 1H), 7.03 (ddd, J=8.8, 7.4, 3.5 Hz, 1H), 6.95 (ddd, J=9.0, 5.5, 3.3 Hz, 1H), 4.31-4.24 (m, 1H), 4.16 (dd, J=13.4, 2.9 Hz, 1H), 3.89-3.75 (m, 2H), 3.70 (dtt, J=11.6, 8.1, 3.9 Hz, 1H), 3.40 (d, J=12.8 Hz, 1H), 3.28 (d, J=3.5 Hz, 1H), 2.85 (s, 1H), 2.79-2.67 (m, 2H), 1.89 (td, J=10.4, 3.0 Hz, 3H), 1.81 (s, 1H), 1.76 (s, 3H), 1.65 (d, J=18.1 Hz, 2H), 1.60-1.45 (m, 3H), 1.44-1.26 (m, 4H), 1.22 (dt, J=13.9, 7.1 Hz, 2H).

Example 173 was prepared similarly to Example 164 except using 3-(((R)-7-((2S,4R)-4-amino-2-(2,5-difluorophenyl)piperidine-1-carbonyl)-7-azaspiro[4.5]decan-10-yl)methyl)-6-fluoroquinazolin-4(3H)-one instead of 3-(((R)-7-((2S,4R)-4-amino-2-(2,5-difluorophenyl)piperidine-1-carbonyl)-7-azaspiro[4.5]decan-10-yl)methyl)-6-phenylpyrimidin-4(3H)-one [Example 86].

### Example 174: 3-(((R)-7-((2S,4R)-2-(2,5-Difluorophenyl)-4-(dimethylamino)piperidine-1-carbonyl)-7-azaspiro[4.5]decan-10-yl)methyl)-6-fluoroquinazolin-4(3H)-one

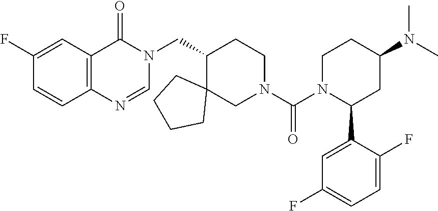 [SMILES: CN(C)[C@@H]1CCN(C(=O)N2CC[C@@H](Cn3cnc4ccc(F)cc4c3=O)C3(CCCC3)C2)[C@H](c2cc(F)ccc2F)C1]

To a stirred solution of 3-(((R)-7-((2S,4R)-4-Amino-2-(2,5-difluorophenyl)piperidine-1-carbonyl)-7-azaspiro[4.5]decan-10-yl)methyl)-6-fluoroquinazolin-4(3H)-one (40 mg, 0.072 mmol) and 37% formaldehyde (aq) solution (108 μL, 1.45 mmol) in 1:1 MeCN/MeOH (1.0 mL) was added sodium triacetoxyborohydride (306 mg, 1.45 mmol) portionwise at rt. After 18 h, the reaction mixture was quenched with saturated NaHCO_{3(aq)} and extracted with DCM (×3) using a phase separator. The combined organic phase was concentrated in vacuo and the resulting residue was purified by flash chromatography using a Biotage Sfar Amino D column (10-100% EtOAc in cyclohexane; then 0-10% MeOH in EtOAc) to give the title compound (23.6 mg, 56%). LCMS (Method B): R_{T}=0.91 min, m/z=582 [M+H]⁺. ¹H NMR (500 MHz, DMSO-d₆): δ 8.37 (s, 1H), 7.82 (dd, J=8.7, 2.9 Hz, 1H), 7.79-7.69 (m, 2H), 7.13 (td, J=9.7, 4.6 Hz, 1H), 7.02 (ddd, J=7.9, 6.1, 3.1 Hz, 2H), 4.27-4.21 (m, 1H), 4.15 (dd, J=13.3, 3.0 Hz, 1H), 3.89-3.76 (m, 2H), 3.39 (d, J=12.9 Hz, 1H), 3.35-3.32 (m, 1H), 2.86 (s, 1H), 2.75 (d, J=12.9 Hz, 1H), 2.66 (d, J=11.5 Hz, 1H), 2.32 (dd, J=14.0, 5.6 Hz, 1H), 2.16 (s, 6H), 1.92-1.73 (m, 4H), 1.71-1.44 (m, 5H), 1.43-1.15 (m, 6H).

### Example 175: (2S4R)-1-((R)-10-((6-Oxo-4-phenylpyrimidin-1(6H)-yl)methyl)-7-azaspiro[4.5]decane-7-carbonyl)-2-phenylpiperidine-4-carboxylic acid

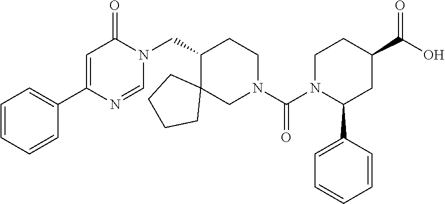 [SMILES: O=C(O)[C@@H]1CCN(C(=O)N2CC[C@@H](Cn3cnc(-c4ccccc4)cc3=O)C3(CCCC3)C2)[C@H](c2ccccc2)C1]

To a stirred solution of methyl (2S,4R)-1-((R)-10-((6-oxo-4-phenylpyrimidin-1(6H-yl)methyl)-7-azaspiro[4.5]decane-7-carbonyl)-2-phenylpiperidine-4-carboxylate (90.3 mg, 0.159 mmol) [Example 76] in anhydrous THF (0.90 mL) was added a solution of lithium hydroxide (7.6 mg, 0.318 mmol) in water (0.30 mL) at rt. After 16 h, the mixture was partitioned with 2 M hydrochloric acid (2 mL). The aqueous phase was separated and extracted using DCM (2×1 mL). The combined organic phase was dried (phase separator) and concentrated in vacuo to give the crude product that was purified by flash chromatography (0-100% EtOAc+1% (v/v) acetic acid in cyclohexane) and freeze-dried to afford the title compound (94.5 mg, quantitative). LCMS (Method B): R_{T}=1.31 min, m/z=555 [M+H]⁺. ¹H NMR (500 MHz, DMSO-d₆): δ 8.55 (s, 1H), 8.09-8.02 (m, 2H), 7.52-7.46 (m, 3H), 7.25-7.17 (m, 4H), 7.17-7.11 (m, 1H), 6.97 (s, 1H), 4.10 (dd, 1H), 3.95 (dd, 1H), 3.92-3.84 (m, 1H), 3.70 (t, 1H), 3.44 (d, 1H), 2.91-2.77 (m, 1H), 2.72-2.61 (m, 2H), 2.43-2.36 (m, 1H), 1.97-1.81 (m, 3H), 1.81-1.72 (m, 1H), 1.72-1.59 (m, 3H), 1.59-1.42 (m, 3H), 1.39-1.13 (m, 6H). OH signal not observed.

Example 176 was prepared similarly to Example 96 except using (2S,4R)-1-((R)-10-((6-oxo-4-phenylpyrimidin-1(6H)-yl)methyl)-7-azaspiro[4.5]decane-7-carbonyl)-2-phenylpiperidine-4-carboxylic acid and methylamine instead of 1-(((R)-7-((2S,4R)-4-amino-2-phenylpiperidine-1-carbonyl)-7-azaspiro[4.5]decan-10-yl)methyl)-4-phenylpyridin-2(1H)-one and cyclopropanecarboxylic acid, respectively. Example 177 was prepared similarly to Example 96 except using (2S,4R)-1-((R)-10-((6-oxo-4-phenylpyrimidin-1(6H)-yl)methyl)-7-azaspiro[4.5]decane-7-carbonyl)-2-phenylpiperidine-4-carboxylic acid and dimethylamine instead of 1-(((R)-7-((2S,4R)-4-amino-2-phenylpiperidine-1-carbonyl)-7-azaspiro[4.5]decan-10-yl)methyl)-4-phenylpyridin-2(1H)-one and cyclopropanecarboxylic acid, respectively. Example 178 was prepared similarly to Example 168 except using Intermediate 54 instead of Intermediate 53 and the separation of diastereoisomers step was not required. Example 179 was prepared similarly to Example 123 except using Intermediate 49 Instead of Intermediate 19. Example 180 was prepared similarly to Example 91 except using Intermediate 49 Instead of Intermediate 19. Example 181 was prepared similarly to Example 153 except using Intermediate 30 instead of Intermediate 35. Example 182 was prepared similarly to Example 163 except using Intermediate 42 instead of Intermediate 19. Example 183 was prepared similarly to Example 152 except using Intermediate 55 instead of Intermediate 42. Example 184 was prepared similarly to Example 174 except using 3-(((R)-7-((2S,4R)-4-amino-2-(3,5-difluorophenyl)piperidine-1-carbonyl)-7-azaspiro[4.5]decan-10-yl)methyl)-6-fluoroquinazolin-4(3H)-one [Prepared according to Example 172 Steps 1-3 except using Intermediate 30 instead of tert-butyl ((2S,4R)-2-(2,5-difluorophenyl)piperidin-4-yl)carbamate and General Procedure 6 instead of General Procedure 1 for the Step 3] instead of 3-(((R)-7-((2S,4R)-4-amino-2-(2,5-difluorophenyl)piperidine-1-carbonyl)-7-azaspiro[4.5]decan-10-yl)methyl)-6-fluoroquinazolin-4(3H)-one. Example 185 was prepared similarly to Example 123 except using Intermediate 42 Instead of Intermediate 19. Example 186 was prepared similarly to Example 123 except using Intermediate 55 Instead of Intermediate 19.

### Example 187: 3-(((S)-7-((R)-2-(2,5-Difluorophenyl)piperazine-1-carbonyl)-10-methoxy-7-azaspiro[4.5]decan-10-yl)methyl)-6-(2-methoxyphenyl)pyrimidin-4(3H)-one

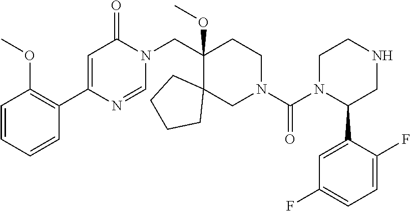 [SMILES: COc1ccccc1-c1cc(=O)n(C[C@]2(OC)CCN(C(=O)N3CCNC[C@H]3c3cc(F)ccc3F)CC23CCCC3)cn1]

Step 1: tert-Butyl (S)-10-methoxy-10-((4-(2-methoxyphenyl)-6-oxopyrimidin-1(6H)-yl)methyl)-7-azaspiro[4.5]decane-7-carboxylate: To a stirred solution of tert-butyl (S)-10-hydroxy-10-((4-(2-methoxyphenyl)-6-oxopyrimidin-1(6H)-yl)methyl)-7-azaspiro[4.5]decane-7-carboxylate (400 mg, 0.852 mmol) in DMF (8 mL) cooled to 0° C. was added sodium hydride (60% dispersion in oil, 68.1 mg, 1.70 mmol) in portions. The resulting suspension was stirred at 0° C. for 30 min before adding dimethylsulfate (121 μL, 1.28 mmol). The reaction was stirred at 0° C. for 2 h before quenching with saturated NaHCO_{3(aq)} and the resulting mixture was extracted with DCM (×3) using a phase separator. The combined organic phase was concentrated in vacuo and the resulting residue was purified by flash chromatography (0-100% EtOAc in cyclohexane) to give the title compound (130 mg, 31%). LCMS (Method B): R_{T}=1.66 min, m/z=484 [M+H]⁺.

Step 2: (S)-3-((10-Methoxy-7-azaspiro[4.5]decan-10-yl)methyl)-6-(2-methoxyphenyl)pyrimidin-4(3H)-one: Prepared according to General Procedure 1 using tert-butyl (S)-10-methoxy-10-((4-(2-methoxyphenyl)-6-oxopyrimidin-1(6H)-yl)methyl)-7-azaspiro[4.5]decane-7-carboxylate (130 mg, 0.269 mmol) and TFA (0.5 mL) in DCM (1 mL) to give the title compound (91 mg, 88%). LCMS (Method B): R_{T}=0.81 min, m/z=384 [M+H]⁺.

Step 3: tert-Butyl (R)-3-(2,5-difluorophenyl)-4-((S)-10-methoxy-10-((4-(2-methoxyphenyl)-6-oxopyrimidin-1(6H)-yl)methyl)-7-azaspiro[4.5]decane-7-carbonyl)piperazine-1-carboxylate: Prepared according to General Procedure 8 using triphosgene (15.5 mg, 0.052 mmol), pyridine (42 μL, 0.522 mmol) and tert-butyl (R)-3-(2,5-difluorophenyl)piperazine-1-carboxylate (18.7 mg, 0.063 mmol) in DCM (4 mL) to produce the carbamoyl chloride which was subsequently used in General Procedure 7 with (S)-3-((10-methoxy-7-azaspiro[4.5]decan-10-yl)methyl)-6-(2-methoxyphenyl)pyrimidin-4(3H)-one (20 mg, 0.052 mmol) and DIPEA (27 μL, 0.157 mmol) in DCM (1 mL) to give the title compound (12 mg, 32%). LCMS (Method B): R_{T}=1.72 min, m/z=708 [M+H]⁺.

Step 4: 3-(((S)-7-((R)-2-(2,5-Difluorophenyl)piperazine-1-carbonyl)-10-methoxy-7-azaspiro[4.5]decan-10-yl)methyl)-6-(2-methoxyphenyl)pyrimidin-4(3H)-one: Prepared according to General procedure 1 using tert-butyl (R)-3-(2,5-difluorophenyl)-4-((S)-10-methoxy-10-((4-(2-methoxyphenyl)-6-oxopyrimidin-1(6H)-yl)methyl)-7-azaspiro[4.5]decane-7-carbonyl)piperazine-1-carboxylate (12 mg, 0.017 mmol) and TFA (0.5 mL in DCM (1 mL to give the title compound (10 mg, quantitative). LCMS (Method B): R_{T}=1.01 min, m/z=608 [M+H]⁺. ¹H NMR (500 MHz, DMSO-d₆): δ 8.31 (s, 1H), 8.02 (dd, J=7.8, 1.8 Hz, 1H), 7.47 (ddd, J=8.8, 7.3, 1.8 Hz, 1H), 7.17 (ddt, J=14.0, 9.3, 5.2 Hz, 3H), 7.10-7.03 (m, 3H), 4.47 (d, J=14.2 Hz, 1H), 4.36 (dd, J=9.1, 3.5 Hz, 1H), 3.98 (d, J=14.2 Hz, 1H), 3.89 (s, 3H), 3.65 (s, 2H), 3.35 (d, J=13.5 Hz, 1H), 3.16 (s, 4H), 2.97-2.71 (m, 5H), 2.68-2.61 (m, 1H (obscured by DMSO satellite)), 1.67 (ddd, J=35.3, 11.8, 6.2 Hz, 2H), 1.56 (dt, J=16.3, 6.2 Hz, 3H), 1.43 (dq, J=19.1, 9.1 Hz, 3H), 1.19 (d, J=13.0 Hz, 1H), 1.05-0.97 (m, 1H). NH signal not observed.

Example 188 was prepared similarly to Example 68 except using (R)-1-((7-azaspiro[4.5]decan-10-yl)methyl)-4-phenylpyridin-2(1H)-one instead of (R)-3-((7-azaspiro[4.5]decan-10-yl)methyl)-6-phenylpyrimidin-4(3H)-one and Intermediate 56 instead of Intermediate 13. Example 189 was prepared similarly to Example 101 except using Intermediate 50 instead of Intermediate 6.

### Example 190: 3-(((S)-7-((2S,4R)-2-(2,5-Difluorophenyl)-4-(isopropylamino)piperidine-1-carbonyl)-10-methoxy-7-azaspiro[4.5]decan-10-yl)methyl)-6-(2-methoxyphenyl)pyrimidin-4(3H)-one

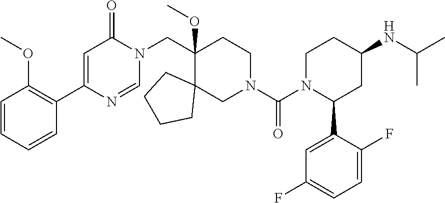 [SMILES: COc1ccccc1-c1cc(=O)n(C[C@]2(OC)CCN(C(=O)N3CC[C@@H](NC(C)C)C[C@H]3c3cc(F)ccc3F)CC23CCCC3)cn1]

Prepared according to General Procedure 8 using triphosgene (27 mg, 0.091 mmol), pyridine (74 μL, 0.913 mmol) and (S)-3-((10-methoxy-7-azaspiro[4.5]decan-10-yl)methyl)-6-(2-methoxyphenyl)pyrimidin-4(3H)-one (35 mg, 0.091 mmol) in DCM (4 mL) to produce the carbamoyl chloride which was subsequently used in General Procedure 7 with (2S,4R)-2-(2,5-difluorophenyl)-N-isopropylpiperidin-4-amine hydrochloride (32 mg, 0.110 mmol) and DIPEA (80 μL, 0.456 mmol) in MeCN (2 mL) heated to 50° C. for 72 h to give the title compound (25.1 mg, 40%). LCMS (Method B): R_{T}=1.02 min, m/z=664 [M+H]⁺. ¹H NMR (500 MHz, DMSO-d₆): δ 8.31 (s, 1H), 8.03 (dd, J=7.8, 1.8 Hz, 1H), 7.47 (ddd, J=8.7, 7.3, 1.8 Hz, 1H), 7.20-7.11 (m, 2H), 7.11-6.98 (m, 4H), 4.49 (d, J=14.1 Hz, 1H), 4.24 (d, J=11.4 Hz, 1H), 3.97 (d, J=14.2 Hz, 1H), 3.90 (s, 3H), 3.72-3.62 (m, 1H), 3.40 (d, J=12.8 Hz, 1H), 3.29-3.21 (m, 1H), 3.15 (s, 3H), 2.99-2.80 (m, 2H), 2.73-2.62 (m, 2H, obscured by DMSO satellite), 2.46-2.38 (m, 1H, obscured by DMSO signal), 1.90 (d, J=12.4 Hz, 2H), 1.75-1.67 (m, 1H), 1.67-1.59 (m, 1H), 1.59-1.30 (m, 7H), 1.30-1.12 (m, 3H), 0.98-0.88 (m, 7H).

Example 191 was prepared similarly to Example 101 except using Intermediate 26 instead of Intermediate 6.

### Example 192: 3-(((R)-7-((2S,4R)-2-(3,5-Difluorophenyl)-4-(isopropylamino)piperidine-1-carbonyl)-7-azaspiro[4.5]decan-10-yl)methyl)-6-phenylpyrimidin-4(3H)-one

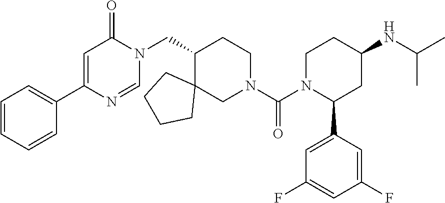 [SMILES: CC(C)N[C@@H]1CCN(C(=O)N2CC[C@@H](Cn3cnc(-c4ccccc4)cc3=O)C3(CCCC3)C2)[C@H](c2cc(F)cc(F)c2)C1]

To a stirred solution of 3-[[(10R)-7-[(2S,4R)-4-amino-2-(3,5-difluorophenyl)piperidine-1-carbonyl]-7-azaspiro[4.5]decan-10-yl]methyl]-6-phenyl-pyrimidin-4-one (41.2 mg, 0.073 mmol) [Example 107] and acetone (10.9 μL, 0.147 mmol) in MeOH (1 mL) was added NaBH(OAc)₃ (31.1 mg, 0.147 mmol) and the mixture was stirred for 30 min. Further acetone (10.9 μL, 0.147 mmol) followed by NaBH(OAc)₃ (31.1 mg, 0.147 mmol) were added. After an additional 30 min, further acetone (10.9 uL, 0.147 mmol) followed by NaBH(OAc)₃ (31.1 mg, 0.147 mmol) were added. Further acetone (10.9 uL, 0.147 mmol) followed by NaBH(OAc)₃ (31.1 mg, 0.147 mmol) were added and stirring continued for 30 min. The addition of further acetone (10.9 uL, 0.147 mmol) followed by NaBH(OAc)₃ (31.1 mg, 0.147 mmol) required to be repeated 8 more times until the reaction was sufficiently completed. An SCX-2 silica cartridge (5 g) was pretreated with 20% (v/v) MeOH in DCM (50 mL). The reaction mixture was diluted with MeOH until it became a solution (ca. 0.5 mL) then placed evenly on to the SCX-2 column, using MeOH (3×0.5 mL) to rinse the flask. After 5 min, the column was flushed with 20% (v/v) MeOH in DCM (50 mL) followed by 20% (v/v) (7 M ammonia in MeOH) in DCM (50 mL). The ammonia solution was concentrated in vacuo and the remaining residue was purified by flash chromatography using a KP-NH column (0-100% EtOAc in cyclohexane) and freeze-dried to afford the title compound (32 mg, 71% yield). LCMS (Method K): R_{T}=0.93 min, m/z=604 [M+H]⁺. ¹H NMR (500 MHz, DMSO-d₆): δ 8.55 (s, 1H), 8.10-8.01 (m, 2H), 7.54-7.45 (m, 3H), 7.02-6.93 (m, 2H), 6.92-6.84 (m, 2H), 4.11 (dd 1H), 4.01 (dd, 1H), 3.85 (dt, 1H), 3.72 (t, 1H), 3.41 (d, 1H), 3.28-3.21 (m, 1H), 2.92-2.78 (m, 2H), 2.74 (d, 1H), 2.69-2.60 (m, 2H), 1.95-1.83 (m, 3H), 1.83-1.74 (m, 1H), 1.72-1.61 (m, 2H), 1.61-1.44 (m, 2H), 1.42-1.11 (m, 8H), 0.98-0.88 (m, 6H).

### Example 193: 3-(((R)-7-((2S,4R)-4-(((3-Methylpyridin-2-yl)methyl)amino)-2-phenylpiperidine-1-carbonyl)-7-azaspiro[4.5]decan-10-yl)methyl)-6-phenylpyrimidin-4(3H)-one

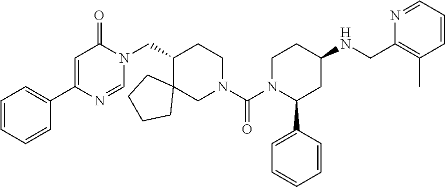 [SMILES: Cc1cccnc1CN[C@@H]1CCN(C(=O)N2CC[C@@H](Cn3cnc(-c4ccccc4)cc3=O)C3(CCCC3)C2)[C@H](c2ccccc2)C1]

To a stirred solution of 3-(((R)-7-((2S,4R)-4-amino-2-phenylpiperidine-1-carbonyl)-7-azaspiro[4.5]decan-10-yl)methyl)-6-phenylpyrimidin-4(3H)-one (70 mg, 0.133 mmol) [Example 25] in MeOH (1 mL) under N₂ was added 3-methylpicolinaldehyde (20.2 mg, 0.167 mmol). The resulting mixture was stirred at rt for 18 h before the portionwise addition of NaBH(OAc)₃ (282 mg, 1.33 mmol) at rt. After 1 h, the reaction was quenched with saturated NaHCO_{3(aq)} and extracted with DCM (×3) using a phase separator. The combined organic phase was concentrated in vacuo and the crude material was purified by flash chromatography using a Biotage Sfar Amino D column (10-100% EtOAc in cyclohexane; then 0-10% MeOH in EtOAc) to give the title compound (43 mg, 50%). LCMS (Method K): R_{T}=0.95 min, m/z=631 [M+H]⁺. ¹H NMR (500 MHz, DMSO-d₆): δ 8.55 (s, 1H), 8.30 (dd, J=4.9, 1.6 Hz, 1H), 8.09-8.03 (m, 2H), 7.50 (dtt, J=8.4, 5.3, 2.5 Hz, 4H), 7.22 (d, J=6.1 Hz, 4H), 7.18-7.10 (m, 2H), 6.97 (s, 1H), 4.10 (dd, J=13.0, 3.1 Hz, 1H), 3.97 (dd, J=11.7, 3.0 Hz, 1H), 3.88 (d, J=13.2 Hz, 1H), 3.80 (t, J=4.8 Hz, 2H), 3.70 (t, J=12.0 Hz, 1H), 3.44 (d, J=12.9 Hz, 1H), 3.29-3.22 (m, 1H), 2.84 (s, 1H), 2.73-2.61 (m, 3H), 2.27 (s, 3H), 2.20 (s, 1H), 1.99 (dd, J=12.7, 4.0 Hz, 2H), 1.85 (ddt, J=10.5, 7.0, 3.4 Hz, 1H), 1.77 (s, 1H), 1.72-1.40 (m, 5H), 1.39-1.24 (m, 4H), 1.19 (td, J=15.9, 14.2, 8.6 Hz, 2H).

Example 194 was prepared similarly to Example 170 except using 3-(((R)-7-((2S,4R)-4-amino-2-phenylpiperidine-1-carbonyl)-7-azaspiro[4.5]decan-10-yl)methyl)-6-phenylpyrimidin-4(3H)-one instead of 3-(((R)-7-((2S,4R)-4-amino-2-(2,5-difluorophenyl)piperidine-1-carbonyl)-7-azaspiro[4.5]decan-10-yl)methyl)-6-phenylpyrimidin-4(3H)-one and 2-(chloromethyl)-4,6-dimethylpyrimidine instead of 4-(chloromethyl)pyrimidine. Example 195 was prepared similarly to Example 78 except using Intermediate 57 instead of Intermediate 19. Example 196 and Example 197 [Note: this was an unexpected by-product during the synthesis of Example 196] were prepared similarly to Example 78 except using Intermediate 58 instead of Intermediate 19. Example 198 was prepared similarly to Example 125 except using Intermediate 59 instead of Intermediate 27. Example 199 was prepared similarly to Example 125 except using Intermediate 60 instead of Intermediate 27. Example 200 was prepared similarly to Example 125 except using Intermediate 61 instead of Intermediate 1 and no deprotection step was required. Example 201 was prepared similarly to Example 200 except using (R)-3-((7-azaspiro[4.5]decan-10-yl)methyl)-6-phenylpyrimidin-4(3H)-one instead of Intermediate 27.

| | | | ¹H NMR | LCMS |
|---|---|---|---|---|
| | | | (500 MHz, DMSO-d₆): | (Method): |
| Example | Structure | Name | δ | R_{T}, m/z |
| 59 | 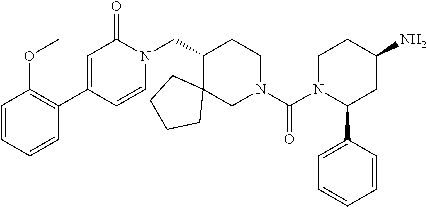 [SMILES: COc1ccccc1-c1ccn(C[C@@H]2CCN(C(=O)N3CC[C@@H](N)C[C@H]3c3ccccc3)CC23CCCC3)c(=O)c1] | 1-(((R)-7-((2S,4R)-4-Amino-2- phenylpiperidine-1-carbonyl)-7- azaspiro[4.5]decan-10-yl)methyl)- 4-(2-methoxyphenyl)pyridin- 2(1H)-one | 7.62 (d, J = 7.0 Hz, 1H), 7.41 (ddd, J = 8.7, 7.4, 1.8 Hz, 1H), 7.34 (dd, J = 7.6, 1.8 Hz, 1H), 7.24-7.16 (m, 4H), 7.16- 7.10 (m, 2H), 7.03 (td, J = 7.5, 1.1 Hz, 1H), 6.46 (d, J = 1.9 Hz, 1H), 6.36 (dd, J = 7.1, 2.0 Hz, 1H), 4.06 (dd, J = 12.6, 3.0 Hz, 1H), 3.93 (dd, J = 11.7, 3.0 Hz, 1H), 3.91-3.84 (m, 1H), 3.80 (s, 3H), 3.69 (t, J = 11.8 Hz, 1H), 3.41 (d, J = 12.8 Hz, 1H), 3.22 (dt, J = 12.1, 3.6 Hz, 1H), 2.89 (s, 1H), 2.75-2.65 (m, 2H), 2.16-1.71 (m, 6H), 1.70-1.11 (m, 12H). | (Method B) R_{T} = 0.95 min, m/z 555 [M + H]⁺ |
| 60 | 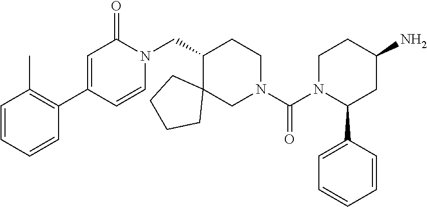 [SMILES: Cc1ccccc1-c1ccn(C[C@@H]2CCN(C(=O)N3CC[C@@H](N)C[C@H]3c3ccccc3)CC23CCCC3)c(=O)c1] | 1-(((R)-7-((2S,4R)-4-Amino-2- phenylpiperidine-1-carbonyl)-7- azaspiro[4.5]decan-10-yl)methyl)-4- (o-tolyl)pyridin-2(1H)-one | 7.68 (d, J = 6.9 Hz, 1H), 7.32-7.28 (m, 2H), 7.26 (td, J = 6.8, 6.0, 2.8 Hz, 1H), 7.24-7.16 (m, 5H), 7.16-7.08 (m, 1H), 6.27 (d, J = 2.0 Hz, 1H), 6.22 (dd, J = 6.9, 2.0 Hz, 1H), 4.09 (dd, J = 12.7, 3.1 Hz, 1H), 3.95-3.84 (m, 2H), 3.72 (t, J = 11.9 Hz, 1H), 3.41 (d, J = 12.7 Hz, 1H), 3.21 (dt, J = 12.1, 3.7 Hz, 1H), 2.91 (s, 1H), 2.69 (dq, J = 14.8, 5.5, 4.4 Hz, 2H), 2.27 (s, 3H), 1.90-1.71 (m, 4H), 1.71-1.42 (m, 6H), 1.42-1.12 (m, 8H). | (Method B) R_{T} = 0.98 min, m/z 539 [M + H]⁺ |
| 61 | 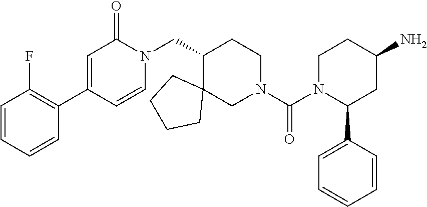 [SMILES: N[C@@H]1CCN(C(=O)N2CC[C@@H](Cn3ccc(-c4ccccc4F)cc3=O)C3(CCCC3)C2)[C@H](c2ccccc2)C1] | 1-(((R)-7-((2S,4R)-4-Amino-2- phenylpiperidine-1- carbonyl)-7-azaspiro[4.5]decan-10- yl)methyl)-4-(2-fluorophenyl)pyridin- 2(1H)-one | 7.73 (d, J = 7.1 Hz, 1H), 7.59 (td, J = 7.8, 1.8 Hz, 1H), 7.50 (dddd, J = 8.5, 7.2, 5.2, 1.8 Hz, 1H), 7.36-7.29 (m, 2H), 7.24- 7.16 (m, 4H), 7.16-7.10 (m, 1H), 6.54 (t, J = 1.5 Hz, 1H), 6.42 (dt, J = 7.0, 2.0 Hz, 1H), 4.09 (dd, J = 12.8, 3.1 Hz, 1H), 3.93 (dd, J = 11.8, 3.0 Hz, 1H), 3.87 (dd, J = 11.3, 6.6 Hz, 1H), 3.71 (t, J = 11.8 Hz, 1H), 3.42 (d, J = 12.8 Hz, 1H), 3.21 (dt, J = 12.1, 3.7 Hz, 1H), 2.88 (s, 1H), 2.76- 2.65 (m, 2H), 1.83 (ddt, J = 22.6, 12.7, 6.2 Hz, 6H), 1.71-1.11 (m, 12H). | (Method B) R_{T} = 0.94 min, m/z 543 [M + H]⁺ |
| 62 | 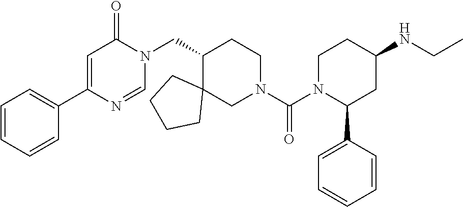 [SMILES: CCN[C@@H]1CCN(C(=O)N2CC[C@@H](Cn3cnc(-c4ccccc4)cc3=O)C3(CCCC3)C2)[C@H](c2ccccc2)C1] | 3-(((R)-7-((2S,4R)-4-(Ethylamino)-2- phenylpiperidine-1-carbonyl)-7- azaspiro[4.5]decan-10-yl)methyl)-6- phenylpyrimidin-4(3H)- one | 8.55 (d, J = 0.9 Hz, 1H), 8.09-8.03 (m, 2H), 7.49 (dd, J = 5.1, 1.9 Hz, 3H), 7.24- 7.16 (m, 4H), 7.15-7.11 (m, 1H), 6.97 (d, J = 0.8 Hz, 1H), 4.10 (dd, J = 13.1, 3.1 Hz, 1H), 3.95 (dd, J = 11.7, 3.1 Hz, 1H), 3.87 (d, J = 13.3 Hz, 1H), 3.70 (t, J = 12.0 Hz, 1H), 3.44 (d, J = 12.9 Hz, 1H), 3.25 (dt, J = 12.2, 3.8 Hz, 1H), 2.83 (s, 1H), 2.71-2.64 (m, 2H, obscured by DMSO satellite), 2.60-2.53 (m, 1H, obscured by DMSO signal), 2.53-2.51 (m, 2H, obscured by DMSO signal), 1.95-1.81 (m, 3H), 1.77 (s, 1H), 1.71-1.43 (m, 4H), | (Method B) R_{T} = 0.95 min, m/z 554 [M + H]⁺ |
| | | | 1.43-1.14 (m, 8H), 0.96 (t, J = 7.1 Hz, | |
| | | | 3H). | |
| 63 | 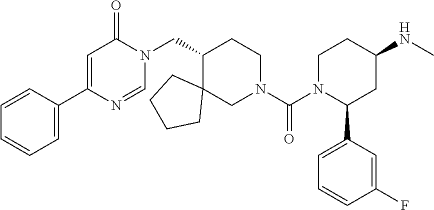 [SMILES: CN[C@@H]1CCN(C(=O)N2CC[C@@H](Cn3cnc(-c4ccccc4)cc3=O)C3(CCCC3)C2)[C@H](c2cccc(F)c2)C1] | 3-(((R)-7-((2S,4R)-2-(3-Fluorophenyl)-4- (methylamino)piperidine-1-carbonyl)-7- azaspiro[4.5]decan-10-yl)methyl)-6- phenylpyrimidin-4(3H)- one | 8.55 (d, J = 0.9 Hz, 1H), 8.09-8.03 (m, 2H), 7.49 (dd, J = 5.1, 1.9 Hz, 3H), 7.30- 7.23 (m, 1H), 7.04 (dt, J = 7.7, 1.2 Hz, 1H), 7.00-6.93 (m, 3H), 4.10 (dd, J = 13.1, 3.1 Hz, 1H), 3.99 (dd, J = 11.8, 3.0 Hz, 1H), 3.90-3.83 (m, 1H), 3.71 (t, J = 12.0 Hz, 1H), 3.42 (d, J = 12.9 Hz, 1H), 3.26 (dt, J = 11.9, 3.7 Hz, 1H), 2.84 (s, 1H), 2.74-2.61 (m, 2H, obscured by DMSO satellite), 2.46-2.38 (m, 1H, obscured by DMSO signal), 2.24 (s, 3H), 1.95-1.82 (m, 3H), 1.77 (s, 1H), 1.71- 1.44 (m, 5H), 1.39-1.13 (m, 7H). | (Method B) R_{T} = 0.96 min, m/z 558 [M + H]⁺ |
| 64 | 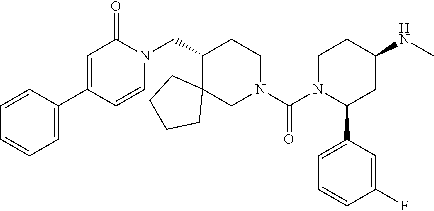 [SMILES: CN[C@@H]1CCN(C(=O)N2CC[C@@H](Cn3ccc(-c4ccccc4)cc3=O)C3(CCCC3)C2)[C@H](c2cccc(F)c2)C1] | 1-(((R)-7-((2S,4R)-2-(3-Fluorophenyl)-4- (methylamino)piperidine-1-carbonyl)-7- azaspiro[4.5]decan-10-yl)methyl)-4- phenylpyridin-2(1H)-one | 7.75-7.68 (m, 3H), 7.51-7.43 (m, 3H), 7.26 (td, J = 7.8, 6.1 Hz, 1H), 7.04 (dt, J = 7.7, 1.2 Hz, 1H), 6.99-6.92 (m, 2H), 6.66 (d, J = 2.1 Hz, 1H), 6.57 (dd, J = 7.1, 2.1 Hz, 1H), 4.09 (dd, J = 12.8, 3.1 Hz, 1H), 3.99 (dd, J = 11.7, 3.0 Hz, 1H), 3.89- 3.82 (m, 1H), 3.71 (t, J = 11.9 Hz, 1H), 3.40 (d, J = 12.8 Hz, 1H), 3.26 (dt, J = 12.2, 4.4 Hz, 1H), 2.87 (s, 1H), 2.75- 2.64 (m, 2H), 2.42 (td, J = 10.6, 5.3 Hz, 1H), 2.24 (s, 3H), 1.95-1.71 (m, 4H), 1.70-1.42 (m, 5H), 1.40-1.11 (m, 7H). | (Method B) R_{T} = 0.97 min, m/z 557 [M + H]⁺ |
| 65 | 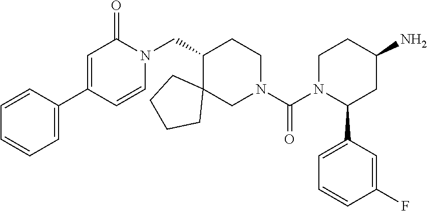 [SMILES: N[C@@H]1CCN(C(=O)N2CC[C@@H](Cn3ccc(-c4ccccc4)cc3=O)C3(CCCC3)C2)[C@H](c2cccc(F)c2)C1] | 1-(((R)-7-((2S,4R)-4-Amino-2-(3- fluorophenyl)piperidine-1-carbonyl)-7- azaspiro[4.5]decan-10-yl)methyl)-4- phenylpyridin-2(1H)-one | 7.76-7.67 (m, 3H), 7.52-7.43 (m, 3H), 7.29-7.22 (m, 1H), 7.03 (dt, J = 7.7, 1.2 Hz, 1H), 6.98-6.91 (m, 2H), 6.66 (d, J = 2.1 Hz, 1H), 6.58 (dd, J = 7.1, 2.1 Hz, 1H), 4.09 (dd, J = 12.8, 3.1 Hz, 1H), 3.96 (dd, J = 11.8, 3.0 Hz, 1H), 3.90-3.82 (m, 1H), 3.71 (t, J = 10.9 Hz, 1H), 3.40 (d, J = 12.9 Hz, 1H), 3.22 (dt, J = 12.2, 3.7 Hz, 1H), 2.88 (s, 1H), 2.76-2.59 (m, 3H, obscured by DMSO satellite), 1.91-1.72 (m, 4H), 1.72-1.41 (m, 6H), 1.41-1.14 (m, 7H). | (Method B) R_{T} = 0.96 min, m/z 543 [M + H]⁺ |
| 66 | 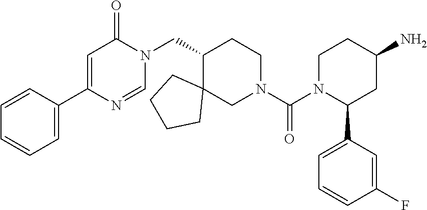 [SMILES: N[C@@H]1CCN(C(=O)N2CC[C@@H](Cn3cnc(-c4ccccc4)cc3=O)C3(CCCC3)C2)[C@H](c2cccc(F)c2)C1] | 3-(((R)-7-((2,4R)-4-Amino-2-(3- fluorophenyl)piperidine-1-carbonyl)-7- azaspiro[4.5]decan-10-yl)methyl)-6- phenylpyrimidin-4(3H)-one | 8.55 (s, 1H), 8.10-8.03 (m, 2H), 7.52- 7.46 (m, 3H), 7.29-7.23 (m, 1H), 7.03 (d, J = 7.9 Hz, 1H), 6.99-6.92 (m, 3H), 4.10 (dd, J = 13.1, 3.1 Hz, 1H), 3.96 (dd, J = 11.8, 3.0 Hz, 1H), 3.87 (d, J = 13.4 Hz, 1H), 3.71 (t, J = 12.0 Hz, 1H), 3.43 (d, J = 12.9 Hz, 1H), 3.22 (dt, J = 12.1, 3.7 Hz, 1H), 2.85 (s, 1H), 2.75-2.59 (m, 3H, obscured by DMSO satellite), 1.91-1.72 (m, 4H), 1.71-1.42 (m, 6H), 1.42-1.14 (m, 7H). | (Method B) R_{T} = 0.94 min, m/z 544 [M + H]⁺ |
| 67 | 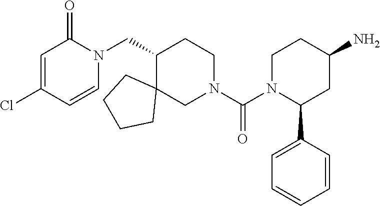 [SMILES: N[C@@H]1CCN(C(=O)N2CC[C@@H](Cn3ccc(Cl)cc3=O)C3(CCCC3)C2)[C@H](c2ccccc2)C1] | 1-(((R)-7-((2S,4R)-4-Amino-2- phenylpiperidine-1-carbonyl)-7- azaspiro[4.5]decan-10-yl)methyl)-4- chloropyridin-2(1H)-one | 7.74 (d, J = 7.3 Hz, 1H), 7.25-7.10 (m, 5H), 6.52 (d, J = 2.4 Hz, 1H), 6.35 (dd, J = 7.3, 2.4 Hz, 1H), 4.04 (dd, J = 12.8, 3.1 Hz, 1H), 3.92 (dd, J = 11.7, 2.9 Hz, 1H), 3.85 (dt, J = 13.3, 4.4 Hz, 1H), 3.66 (t, J = 11.9 Hz, 1H), 3.40 (d, J = 12.8 Hz, 1H), 3.20 (dt, J = 12.4, 3.7 Hz, 1H), 2.83 (s, 1H), 2.71 (s, 1H), 2.67-2.59 (m, 2H, obscured by DMSO satellite), 2.19-1.68 (m, 5H), 1.68-1.09 (m, 12H). | (Method B) R_{T} = 0.80 min, m/z 483, 485 [M + H]⁺ |
| 68 | 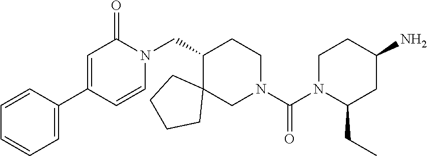 [SMILES: CC[C@@H]1C[C@H](N)CCN1C(=O)N1CC[C@@H](Cn2ccc(-c3ccccc3)cc2=O)C2(CCCC2)C1] | 1-(((R)-7-((2R,4R)-4-Amino-2- ethylpiperidine-1-carbonyl)-7- azaspiro[4.5]decan-10-yl)methyl)-4- phenylpyridin-2(1H)-one | 7.77-7.69 (m, 3H), 7.51-7.43 (m, 3H), 6.66 (d, J = 2.1 Hz, 1H), 6.58 (dd, J = 7.1, 2.1 Hz, 1H), 4.11 (dd, J = 12.7, 3.1 Hz, 1H), 3.79-3.66 (m, 2H), 3.44 (d, J = 12.8 Hz, 1H), 3.12 (ddd, J = 12.6, 5.6, 3.8 Hz, 1H), 2.91 (td, J = 8.4, 4.3 Hz, 1H), 2.72 (dddd, J = 22.2, 12.7, 9.0, 3.4 Hz, 3H), 1.87 (tt, J = 10.3, 3.7 Hz, 3H), 1.77-1.47 (m, 7H), 1.47-1.14 (m, 9H), 0.77 (t, J = 7.4 Hz, 3H). [NH signal not observed]. | (Method B) R_{T} = 0.89 min, m/z 477 [M + H]⁺ |
| 69 | 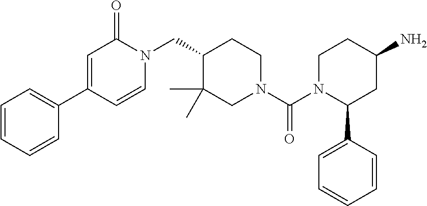 [SMILES: CC1(C)CN(C(=O)N2CC[C@@H](N)C[C@H]2c2ccccc2)CC[C@H]1Cn1ccc(-c2ccccc2)cc1=O] | 1-(((R)-1-((2S,4R)-4-Amino-2- phenylpiperidine-1-carbonyl)-3,3- dimethylpiperidin-4-yl)methyl)-4- phenylpyridin-2(1H)-one | 7.73-7.68 (m, 3H), 7.51-7.43 (m, 3H), 7.23-7.16 (m, 4H), 7.15-7.09 (m, 1H), 6.65 (d, J = 2.0 Hz, 1H), 6.57 (dd, J = 7.1, 2.1 Hz, 1H), 4.16 (dd, J = 12.7, 3.6 Hz, 1H), 4.00 (d, J = 13.2 Hz, 1H), 3.93 (dd, J = 11.7, 3.1 Hz, 1H), 3.59-3.48 (m, 2H), 3.25 (dt, J = 12.1, 3.6 Hz, 1H), 2.70 (ddd, J = 15.0, 10.8, 4.1 Hz, 1H), 2.63-2.53 (m, 3H, signal obscured by DMSO peak), 1.84-1.70 (m, 3H), 1.56 (s, 2H), 1.42- 1.26 (m, 2H), 1.26-1.16 (m, 2H), 1.00 (s, 3H), 0.82 (s, 3H). | (Method B) R_{T} = 0.85 min, m/z 499 [M + H]⁺ |
| 70 | 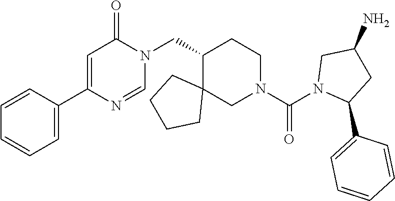 [SMILES: N[C@H]1C[C@@H](c2ccccc2)N(C(=O)N2CC[C@@H](Cn3cnc(-c4ccccc4)cc3=O)C3(CCCC3)C2)C1] | 3-(((R)-7-((2S,4S)-4- Amino-2-phenylpyrrolidine-1- carbonyl)-7-azaspiro[4.5]decan-10- yl)methyl)-6-phenylpyrimidin-4(3H)- one | 8.53 (s, 1H), 8.09-8.02 (m, 2H), 7.49 (dd, J = 5.1, 1.9 Hz, 3H), 7.29-7.20 (m, 4H), 7.20-7.14 (m, 1H), 6.96 (d, J = 0.8 Hz, 1H), 4.89 (dd, J = 11.0, 6.7 Hz, 1H), 4.11 (dd, J = 12.9, 3.0 Hz, 1H), 3.74 (d, J = 13.3 Hz, 1H), 3.62 (dd, J = 13.0, 10.9 Hz, 1H), 3.47 (t, J = 7.0 Hz, 1H), 3.37- 3.23 (m, 4H, obscured by HDO signal), 2.56-2.53 (m, 1H, obscured by DMSO signal), 2.41-2.35 (m, 1H, obscured by DSMO satellite), 1.93 (ddd, J = 13.1, 7.9, 5.1 Hz, 1H), 1.87-1.80 (m, 1H), 1.73- | (Method B) R_{T} = 0.89 min, m/z 512 [M + H]⁺ |
| | | | 1.39 (m, 5H), 1.25 (qd, J = 7.6, 4.0 Hz, | |
| | | | 4H), 1.20-1.14 (m, 1H). [NH₂ signal not | |
| | | | observed] | |
| 71 | 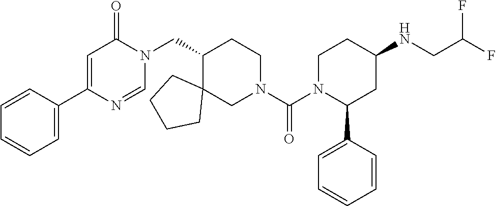 [SMILES: O=C(N1CC[C@@H](Cn2cnc(-c3ccccc3)cc2=O)C2(CCCC2)C1)N1CC[C@@H](NCC(F)F)C[C@H]1c1ccccc1] | 3-(((R)-7-((2S,4R)-4-((2,2- Difluoroethyl)amino)-2- phenylpiperidine-1- carbonyl)-7-azaspiro[4.5]decan- 10-yl)methyl)-6- phenylpyrimidin-4(3H)- one | 8.55 (s, 1H), 8.09-8.03 (m, 2H), 7.49 (dd, J = 5.1, 1.8 Hz, 3H), 7.26-7.17 (m, 4H), 7.17-7.11 (m, 1H), 6.97 (d, J = 0.8 Hz, 1H), 5.89 (tt, J = 56.6, 4.3 Hz, 1H), 4.10 (dd, J = 13.0, 3.1 Hz, 1H), 3.94 (dd, J = 11.7, 3.1 Hz, 1H), 3.88 (d, J = 13.4 Hz, 1H), 3.70 (t, J = 12.0 Hz, 1H), 3.45 (d, J = 12.8 Hz, 1H), 3.25 (dt, J = 12.2, 3.7 Hz, 1H), 2.88 (td, J = 15.9, 4.3 Hz, 3H), 2.64 (d, J = 31.4 Hz, 3H, obscured by DMSO satellite), 2.04-1.72 (m, 5H), 1.72-1.43 (m, 4H), 1.43-1.13 (m, 7H). | (Method B) R_{T} = 0.95 min, m/z 590 [M + H]⁺ |
| 72 | 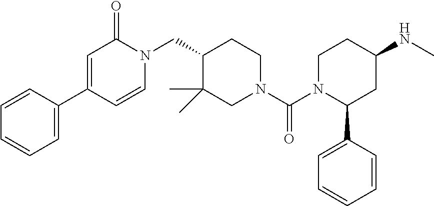 [SMILES: CN[C@@H]1CCN(C(=O)N2CC[C@@H](Cn3ccc(-c4ccccc4)cc3=O)C(C)(C)C2)[C@H](c2ccccc2)C1] | 1-(((R)-3,3-Dimethyl-1- ((2S,4R)-4-(methylamino)-2- phenylpiperidine-1- carbonyl)piperidin-4- yl)methyl)-4- phenylpyridin-2(1H)-one | 7.71 (ddd, J = 7.0, 4.5, 2.7 Hz, 3H), 7.51- 7.43 (m, 3H), 7.23-7.17 (m, 4H), 7.12 (ddd, J = 8.6, 5.7, 2.5 Hz, 1H), 6.65 (d, J = 2.1 Hz, 1H), 6.56 (dd, J = 7.1, 2.1 Hz, 1H), 4.16 (dd, J = 12.7, 3.6 Hz, 1H), 4.04- 3.93 (m, 2H), 3.60-3.47 (m, 2H), 3.27 (t, J = 4.1 Hz, 1H), 2.70-2.64 (m, 1H, obscured by DMSO satellite), 2.62-2.53 (m, 2H, obscured by DMSO satellite), 2.44 (tt, J = 10.6, 4.2 Hz, 1H, obscured by DMSO signal), 2.24 (s, 3H), 1.90 (td, J = 13.6, 7.0 Hz, 2H), 1.74 (tt, J = 10.6, 3.9 Hz, 1H), 1.40-1.25 (m, 2H), 1.25-1.13 | (Method B) R_{T} = 0.86 min, m/z 513 [M + H]⁺ |
| | | | (m, 2H), 1.00 (s, 3H), 0.82 (s, 3H). [NH | |
| | | | signal not observed]. | |
| 73 | 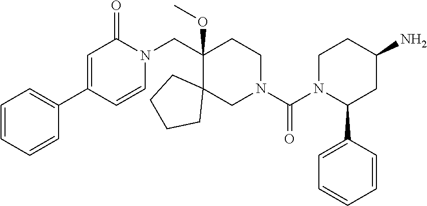 [SMILES: CO[C@@]1(Cn2ccc(-c3ccccc3)cc2=O)CCN(C(=O)N2CC[C@@H](N)C[C@H]2c2ccccc2)CC12CCCC2] | 1-(((S)-7-((2S,4R)-4- Amino-2-phenylpiperidine-1- carbonyl)-10-methoxy-7- azaspiro[4.5]decan-10- yl)methyl)-4- phenylpyridin-2(1H)-one | 7.79-7.70 (m, 2H), 7.58 (d, J = 7.2 Hz, 1H), 7.52-7.44 (m, 3H), 7.26-7.17 (m, 4H), 7.16-7.10 (m, 1H), 6.70 (d, J = 2.1 Hz, 1H), 6.62 (dd, J = 7.2, 2.2 Hz, 1H), 4.48 (d, J = 14.2 Hz, 1H), 4.01-3.87 (m, 2H), 3.77-3.46 (m, 2H), 3.28 (s, 1H), 3.20 (dt, J = 12.1, 3.6 Hz, 1H), 3.15 (s, 3H), 3.00 (s, 1H), 2.71 (td, J = 10.8, 5.3 Hz, 1H), 2.67-2.59 (m, 1H, obscured by DMSO satellite), 1.86-1.78 (m, 2H), 1.74-1.29 (m, 11H), 1.22 (dt, J = 24.6, 8.7 Hz, 2H), 0.90 (s, 1H). | (Method B) R_{T} = 0.99 min, m/z 555 [M + H]⁺ |
| 74 | 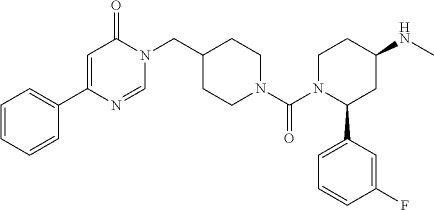 [SMILES: CN[C@@H]1CCN(C(=O)N2CCC(Cn3cnc(-c4ccccc4)cc3=O)CC2)[C@H](c2cccc(F)c2)C1] | 3-((1-((2S,4R)-2-(3- Fluorophenyl)-4- (methylamino)piperidine- 1-carbonyl)piperidin-4- yl)methyl)-6- phenylpyrimidin-4(3H)- one | 8.53 (s, 1H), 8.09-8.04 (m, 2H), 7.52-7.47 (m, 3H), 7.30-7.24 (m, 1H), 7.05-7.01 (m, 1H), 6.99-6.95 (m, 2H), 6.94-6.88 (m, 1H), 4.04-3.91 (m, 3H), 3.87-3.75 (m, 2H), 2.81-2.61 (m, 3.5H), 2.47-2.39 (m, 1H), 2.23 (s, 3H), 2.03-1.86 (m, 3.5H), 1.64-1.48 (m, 2H), 1.37-0.96 (m, 5H). | (Method B) R_{T} = 0.80 min, m/z 504 [M + H]⁺ |
| 75 | 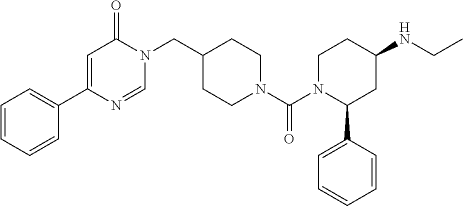 [SMILES: CCN[C@@H]1CCN(C(=O)N2CCC(Cn3cnc(-c4ccccc4)cc3=O)CC2)[C@H](c2ccccc2)C1] | 3-((1-((2S,4R)-4-(Ethylamino)-2- phenylpiperidine-1-carbonyl)piperidin- 4-yl)methyl)-6- phenylpyrimidin-4(3H)- one | 8.53 (s, 1H), 8.09-8.05 (m, 2H), 7.52-7.48 (m, 3H), 7.24-7.15 (m, 4H), 7.10-7.05 (m, 1H), 6.98 (s, 1H), 4.01-3.91 (m, 3H), 3.86- 3.77 (m, 2H), 2.79-2.52 (m, 6H), 2.02- 1.85 (m, 3H), 1.62-1.48 (m, 2H), 1.37- 1.28 (m, 1H), 1.24-0.98 (m, 4H), 0.95 (t, 3H). [NH signal not observed]. | (Method B) R_{T} = 0.80 min, m/z 500 [M + H]⁺ |
| 76 | 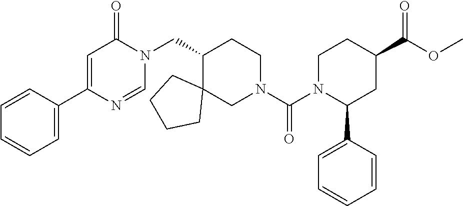 [SMILES: COC(=O)[C@@H]1CCN(C(=O)N2CC[C@@H](Cn3cnc(-c4ccccc4)cc3=O)C3(CCCC3)C2)[C@H](c2ccccc2)C1] | Methyl (2S,4R)-1-((R)-10-((6-oxo-4- phenylpyrimidin-1(6H)- yl)methyl)-7-azaspiro[4.5]decane-7- carbonyl)-2-phenylpiperidine-4- carboxylate | 8.55 (s, 1H), 8.09-8.03 (m, 2H), 7.49 (dd, J = 5.1, 1.9 Hz, 3H), 7.25-7.18 (m, 4H), 7.18-7.13 (m, 1H), 6.97 (s, 1H), 4.10 (dd, J = 13.1, 3.1 Hz, 1H), 4.02 (dd, J = 11.4, 3.2 Hz, 1H), 3.87 (dt, J = 13.2, 4.5 Hz, 1H), 3.71 (dd, J = 13.7, 10.2 Hz, 1H), 3.56 (s, 3H), 3.43 (d, J = 12.9 Hz, 1H), 3.30-3.26 (m, 1H), 2.86 (d, J = 9.8 Hz, 1H), 2.73 (td, J = 12.0, 2.6 Hz, 1H), 2.69-2.56 (m, 2H, obscured by DMSO satellite), 1.99-1.89 (m, 2H), 1.85 (tt, J = 10.6, 3.5 Hz, 1H), 1.81-1.42 (m, 7H), 1.41-1.13 (m, 5H). | (Method B) R_{T} = 1.51 min, m/z 569 [M + H]⁺ |
| 77 | 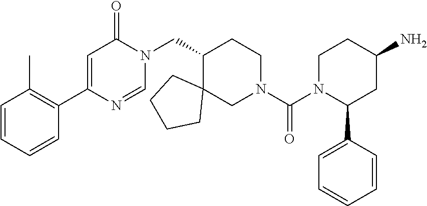 [SMILES: Cc1ccccc1-c1cc(=O)n(C[C@@H]2CCN(C(=O)N3CC[C@@H](N)C[C@H]3c3ccccc3)CC23CCCC3)cn1] | 3-(((R)-7-((2,4R)-4-Amino-2- phenylpiperidine-1-carbonyl)-7- azaspiro[4.5]decan-10- yl)methyl)-6-(o- tolyl)pyrimidin-4(3H)-one | 8.51 (s, 1H), 7.43-7.39 (m, 1H), 7.37-7.32 (m, 1H), 7.31-7.22 (m, 4H), 7.21-7.14 (m, 3H), 6.48-6.46 (m, 1H), 6.29-5.06 (broad s, 2H), 4.17-4.07 (m, 1H), 4.00-3.94 (m, 1H), 3.92-3.85 (m, 1H), 3.77-3.69 (m, 1H), 3.47-3.40 (m, 1H), 3.27-3.23 (m, 1H), 3.05-2.97 (m, 1H), 2.96-2.83 (m, 1H), 2.73-2.65 (m, 2H), 2.38-2.35 (m, 3H), 1.97-1.83 (m, 3H), 1.80-1.13 (m, 12H). | (Method B) R_{T} = 0.87 min, m/z 540 [M + H]⁺ |
| 78 | 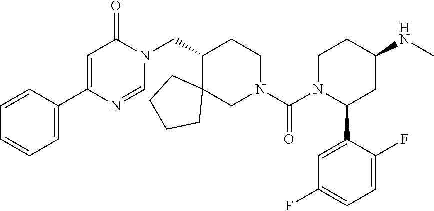 [SMILES: CN[C@@H]1CCN(C(=O)N2CC[C@@H](Cn3cnc(-c4ccccc4)cc3=O)C3(CCCC3)C2)[C@H](c2cc(F)ccc2F)C1] | 3-(((R)-7-((2S,4R)-2-(2,5- Difluorophenyl)-4- (methylamino)piperidine- 1-carbonyl)-7- azaspiro[4.5]decan-10- yl)methyl)-6- phenylpyrimidin-4(3H)- one | 8.55 (s, 1H), 8.09-8.02 (m, 2H), 7.49 (dd, J = 5.1, 1.9 Hz, 3H), 7.14 (td, J = 9.3, 4.5 Hz, 1H), 7.06-6.94 (m, 3H), 4.26 (dt, J = 11.9, 2.1 Hz, 1H), 4.11 (dd, J = 13.1, 3.1 Hz, 1H), 3.86 (dd, J = 11.4, 6.6 Hz, 1H), 3.73 (t, J = 12.2 Hz, 1H), 3.40 (d, J = 12.9 Hz, 1H), 3.32-3.25 (m, 1H, obscured by HDO signal), 2.86 (s, 1H), 2.75 (d, J = 12.9 Hz, 1H), 2.70-2.61 (m, 1H, obscured by DMSO satellite), 2.53- 2.41 (m, 2H, signal obscured by DMSO), 2.25 (s, 3H), 1.99-1.82 (m, 3H), 1.77 (s, 1H), 1.71-1.44 (m, 4H), 1.42-1.14 (m, 7H). | (Method B) R_{T} = 0.96 min, m/z 576 [M + H]⁺ |
| 79 | 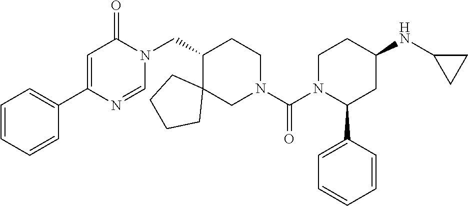 [SMILES: O=C(N1CC[C@@H](Cn2cnc(-c3ccccc3)cc2=O)C2(CCCC2)C1)N1CC[C@@H](NC2CC2)C[C@H]1c1ccccc1] | 3-(((R)-7-((2S,4R)-4- (Cyclopropylamino)-2-phenylpiperidine- 1-carbonyl)-7-azaspiro[4.5]decan-10- yl)methyl)-6-phenylpyrimidin-4(3H)- one | 8.55 (s, 1H), 8.09-8.03 (m, 2H), 7.52-7.46 (m, 3H), 7.24-7.11 (m, 5H), 6.98-6.96 (m, 1H), 4.13-4.06 (m, 1H), 3.97-3.84 (m, 2H), 3.75-3.66 (m, 1H), 3.49-3.40 (m, 1H), 3.27-3.21 (m, 1H), 2.91-2.75 (broad s, 1H), 2.70-2.61 (m, 3H), 2.16 (s, 1H), 2.08-2.02 (m, 1H), 2.00-1.91 (m, 2H), 1.88-1.43 (m, 6H), 1.42-1.13 (m, 7H), 0.38-0.29 (m, 2H), 0.20-0.10 (m, 2H). | (Method B) R_{T} = 0.93 min, m/z 566 [M + H]⁺ |
| 80 | 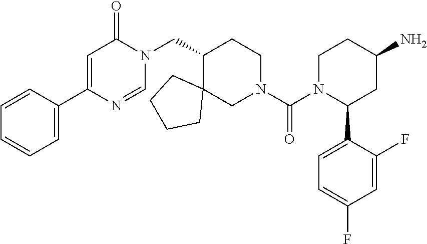 [SMILES: N[C@@H]1CCN(C(=O)N2CC[C@@H](Cn3cnc(-c4ccccc4)cc3=O)C3(CCCC3)C2)[C@H](c2ccc(F)cc2F)C1] | 3-(((R)-7-((2S,4R)-4-Amino-2-(2,4- difluorophenyl)piperidine- 1-carbonyl)-7-azaspiro[4.5]decan-10- yl)methyl)-6-phenylpyrimidin-4(3H)- one | 8.57-8.52 (m, 1H), 8.09-8.03 (m, 2H), 7.49 (dd, J = 5.1, 1.9 Hz, 3H), 7.25 (td, J = 8.5, 6.6 Hz, 1H), 7.08 (ddd, J = 10.7, 9.4, 2.6 Hz, 1H), 6.98-6.91 (m, 2H), 4.21 (dd, J = 12.0, 3.0 Hz, 1H), 4.10 (dd, J = 13.0, 3.1 Hz, 1H), 3.89-3.80 (m, 1H), 3.71 (t, J = 12.0 Hz, 1H), 3.39 (d, J = 12.9 Hz, 1H), 3.23 (dt, J = 12.1, 3.6 Hz, 1H), 2.86 (s, 1H), 2.74-2.61 (m, 3H, obscured by DMSO satellite), 1.90-1.71 (m, 4H), 1.71-1.43 (m, 6H), 1.37 (ddd, J = 20.0, 14.1, 10.0 Hz, 3H), 1.29-1.14 (m, 4H). | (Method B) R_{T} = 0.92 min, m/z 562 [M + H]⁺ |
| 81 | 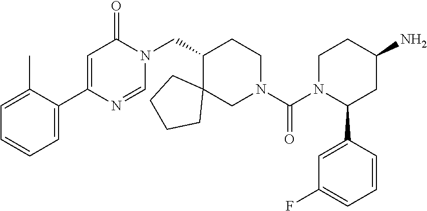 [SMILES: Cc1ccccc1-c1cc(=O)n(C[C@@H]2CCN(C(=O)N3CC[C@@H](N)C[C@H]3c3cccc(F)c3)CC23CCCC3)cn1] | 3-(((R)-7-((2S,4R)-4- Amino-2-(3- fluorophenyl)piperidine- 1-carbonyl)-7- azaspiro[4.5]decan-10- yl)methyl)-6-(0- tolyl)pyrimidin-4(3H)-one | 8.52 (s, 1H), 7.41 (dd, J = 7.6, 1.4 Hz, 1H), 7.34 (td, J = 7.4, 1.5 Hz, 1H), 7.31- 7.23 (m, 3H), 7.06-7.00 (m, 1H), 6.98- 6.93 (m, 2H), 6.46 (s, 1H), 4.11 (dd, J = 13.0, 3.0 Hz, 1H), 3.96 (dd, J = 11.8, 3.0 Hz, 1H), 3.88 (dt, J = 13.2, 4.4 Hz, 1H), 3.73 (t, J = 12.0 Hz, 1H), 3.42 (d, J = 12.7 Hz, 1H), 3.23 (dt, J = 12.2, 3.7 Hz, 1H), 2.89 (s, 1H), 2.76-2.59 (m, 3H, obscured by DMSO satellite), 2.36 (s, 3H, obscured by DMSO satellite), 1.92-1.71 (m, 4H), 1.71-1.43 (m, 6H), 1.43-1.13 (m, 7H). | (Method B) R_{T} = 0.93 min, m/z 558 [M + H]⁺ |
| 82 | 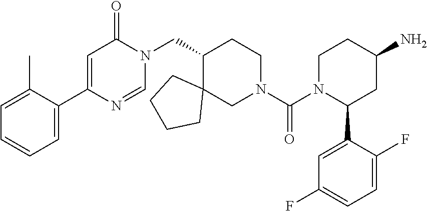 [SMILES: Cc1ccccc1-c1cc(=O)n(C[C@@H]2CCN(C(=O)N3CC[C@@H](N)C[C@H]3c3cc(F)ccc3F)CC23CCCC3)cn1] | 3-(((R)-7-((2S,4R)-4- Amino-2-(2,5- difluorophenyl)piperidine- 1-carbonyl)-7- azaspiro[4.5]decan-10- yl)methyl)-6-(o- tolyl)pyrimidin-4(3H)-one | 8.52 (s, 1H), 7.41 (dd, J = 7.6, 1.4 Hz, 1H), 7.34 (td, J = 7.4, 1.5 Hz, 1H), 7.31- 7.24 (m, 2H), 7.14 (td, J = 9.3, 4.6 Hz, 1H), 7.00 (dddd, J = 21.5, 9.0, 6.5, 3.4 Hz, 2H), 6.46 (s, 1H), 4.23 (d, J = 11.6 Hz, 1H), 4.11 (dd, J = 13.0, 3.0 Hz, 1H), 3.88 (dd, J = 13.2, 4.6 Hz, 1H), 3.74 (t, J = 12.2 Hz, 1H), 3.39 (d, J = 12.6 Hz, 1H), 3.25 (dt, J = 12.2, 3.6 Hz, 1H), 2.91 (s, 1H), 2.76 (d, J = 12.9 Hz, 1H), 2.73- 2.59 (m, 2H, obscured by DMSO satellite), 2.36 (s, 3H, obscured by DMSO satellite), 1.92-1.71 (m, 4H), 1.71-1.44 | (Method B) R_{T} = 0.94 min, m/z 576 [M + H]⁺ |
| | | | (m, 6H), 1.39 (ddt, J = 22.4, 10.5, 5.4 Hz, | |
| | | | 3H), 1.32-1.15 (m, 4H). | |
| 83 | 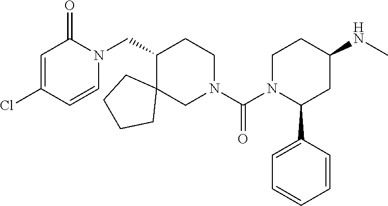 [SMILES: CN[C@@H]1CCN(C(=O)N2CC[C@@H](Cn3ccc(Cl)cc3=O)C3(CCCC3)C2)[C@H](c2ccccc2)C1] | 4-Chloro-1-(((R)-7- ((2S,4R)-4- (methylamino)-2- phenylpiperidine-1- carbonyl)-7- azaspiro[4.5]decan-10- yl)methyl)pyridin-2(1H)- one | 7.73 (d, J = 7.3 Hz, 1H), 7.24-7.15 (m, 4H), 7.15-7.10 (m, 1H), 6.52 (d, J = 2.4 Hz, 1H), 6.35 (dd, J = 7.3, 2.4 Hz, 1H), 4.04 (dd, J = 12.8, 3.1 Hz, 1H), 3.95 (dd, J = 11.7, 3.1 Hz, 1H), 3.84 (dd, J = 11.2, 6.7 Hz, 1H), 3.66 (t, J = 11.8 Hz, 1H), 3.40 (d, J = 12.9 Hz, 1H), 3.23 (dt, J = 12.1, 3.8 Hz, 1H), 2.83 (s, 1H), 2.66 (m, 2H, obscured by DMSO satellite), 2.43 (tt, J = 10.6, 3.8 Hz, 1H), 2.24 (s, 3H), 1.95- 1.85 (m, 2H), 1.77 (ddt, J = 14.8, 11.4, 5.6 Hz, 2H), 1.68-1.39 (m, 5H), 1.28 (tt, J = 22.0, 8.8 Hz, 4H), 1.21-1.09 (m, 3H). | (Method B) R_{T} = 0.82 min, m/z 497, 499 [M + H]⁺ |
| 84 | 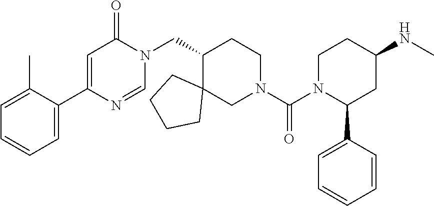 [SMILES: CN[C@@H]1CCN(C(=O)N2CC[C@@H](Cn3cnc(-c4ccccc4C)cc3=O)C3(CCCC3)C2)[C@H](c2ccccc2)C1] | 3-(((R)-7-((2S,4R)-4- (Methylamino)-2- phenylpiperidine-1- carbonyl)-7- azaspiro[4.5]decan-10- yl)methyl)-6-(o- tolyl)pyrimidin-4(3H)-one | 8.54-8.49 (m, 1H), 7.41 (dd, J = 7.6, 1.4 Hz, 1H), 7.34 (td, J = 7.4, 1.5 Hz, 1H), 7.31-7.16 (m, 6H), 7.16-7.10 (m, 1H), 6.46 (d, J = 0.8 Hz, 1H), 4.11 (dd, J = 13.0, 3.0 Hz, 1H), 3.96 (dd, J = 11.7, 3.1 Hz, 1H), 3.89 (d, J = 13.3 Hz, 1H), 3.72 (t, J = 12.0 Hz, 1H), 3.43 (d, J = 12.9 Hz, 1H), 3.26 (dt, J = 12.2, 3.8 Hz, 1H), 2.87 (s, 1H), 2.71-2.65 (m, 2H, obscured by DMSO satellite), 2.47-2.39 (m, 1H), 2.36 (s, 3H, obscured by DMSO satellite), 2.24 (s, 3H), 1.88 (ddd, J = 20.6, 13.0, 9.6 Hz, 3H), 1.76 (s, 1H), 1.71-1.43 (m, 5H), 1.39-1.24 (m, 4H), 1.24-1.14 (m, 3H). | (Method B) R_{T} = 0.91 min, m/z 554 [M + H]⁺ |
| 85 | 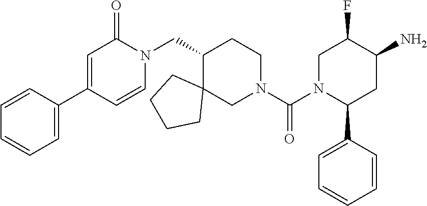 [SMILES: N[C@H]1C[C@@H](c2ccccc2)N(C(=O)N2CC[C@@H](Cn3ccc(-c4ccccc4)cc3=O)C3(CCCC3)C2)C[C@H]1F] | 1-(((R)-7-((2S,4S,5R)-4- Amino-5-fluoro-2- phenylpiperidine-1- carbonyl)-7- azaspiro[4.5]decan-10- yl)methyl)-4- phenylpyridin-2(1H)-one and 1-(((R)-7-((2S,4S,5S)-4- Amino-5-fluoro-2- phenylpiperidine-1- carbonyl)-7- | 7.77-7.69 (m, 3H), 7.51-7.44 (m, 3H), 7.27-7.14 (m, 5H), 6.66 (d, J = 2.1 Hz, 1H), 6.58 (dd, J = 7.1, 2.1 Hz, 1H), 4.55- 4.39 (m, 1H), 4.21 (dd, J = 11.6, 3.5 Hz, 1H), 4.09 (dd, J = 12.8, 3.1 Hz, 1H), 3.83 (dd, J = 11.0, 6.9 Hz, 1H), 3.69 (t, J = 11.7 Hz, 1H), 3.49 (ddd, J = 14.0, 12.1, 4.1 Hz, 1H), 3.36 (d, J = 12.9 Hz, 1H), 2.92 (ddt, J = 19.5, 15.1, 5.6 Hz, 2H), 2.82 (s, 1H), 2.69 (d, J = 12.8 Hz, 1H), 1.95-1.74 (m, 3H), 1.69-1.57 (m, 2H), 1.57-1.43 (m, 3H), 1.43-1.28 (m, 3H), | (Method B) R_{T} = 0.93 min, m/z 543 [M + H]⁺ |
| | | azaspiro[4.5]decan-10- | 1.28-1.13 (m, 4H). | |
| | | yl)methyl)-4- | | |
| | | phenylpyridin-2(1H)-one | | |
| | and | | | |
| | 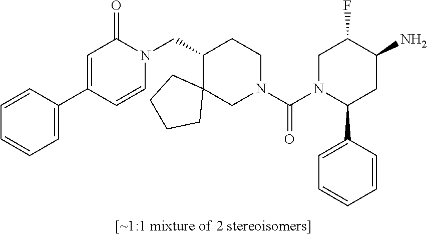 [SMILES: N[C@H]1C[C@@H](c2ccccc2)N(C(=O)N2CC[C@@H](Cn3ccc(-c4ccccc4)cc3=O)C3(CCCC3)C2)C[C@@H]1F] | | | |
| 86 | 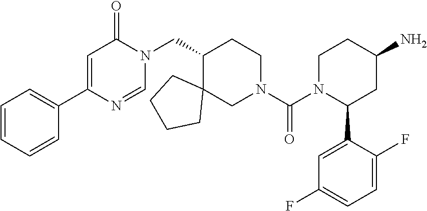 [SMILES: N[C@@H]1CCN(C(=O)N2CC[C@@H](Cn3cnc(-c4ccccc4)cc3=O)C3(CCCC3)C2)[C@H](c2cc(F)ccc2F)C1] | 3-(((R)-7-((2S,4R)-4- Amino-2-(2,5- difluorophenyl)piperidine- 1-carbonyl)-7- azaspiro[4.5]decan-10- yl)methyl)-6- phenylpyrimidin-4(3H)- one | 8.56 (s, 1H), 8.10-8.04 (m, 2H), 7.53-7.47 (m, 3H), 7.17-7.11 (m, 1H), 7.06-6.94 (m, 3H), 4.27-4.21 (m, 1H), 4.15-4.08 (m, 1H), 3.91-3.83 (m, 1H), 3.79-3.68 (m, 1H), 3.47-3.37 (m, 1H), 3.28-3.22 (m, 1H), 2.97-2.59 (m, 4H), 1.93-1.15 (m, 17H). | (Method B) R_{T} = 0.96 min, m/z 562 [M + H]⁺ |
| 87 | 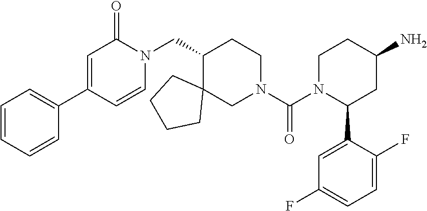 [SMILES: N[C@@H]1CCN(C(=O)N2CC[C@@H](Cn3ccc(-c4ccccc4)cc3=O)C3(CCCC3)C2)[C@H](c2cc(F)ccc2F)C1] | 1-(((R)-7-((2S,4R)-4- Amino-2-(2,5- difluorophenyl)piperidine- 1-carbonyl)-7- azaspiro[4.5]decan-10- yl)methyl)-4- phenylpyridin-2(1H)-one | 7.76-7.69 (m, 3H), 7.51-7.44 (m, 3H), 7.17-7.10 (m, 1H), 7.05-6.94 (m, 2H), 6.67-6.65 (m, 1H), 6.60-6.56 (m, 1H), 4.26-4.20 (m, 1H), 4.13-4.06 (m, 1H), 3.89-3.81 (m, 1H), 3.78-3.67 (m, 1H), 3.27-3.20 (m, 2H), 3.00-2.82 (m, 1H), 2.80-2.59 (m, 3H), 1.91-1.73 (m, 4H), 1.70-1.45 (m, 4H), 1.43-1.14 (m, 9H). | (Method B) R_{T} = 0.95 min, m/z 561 [M + H]⁺ |
| 88 | 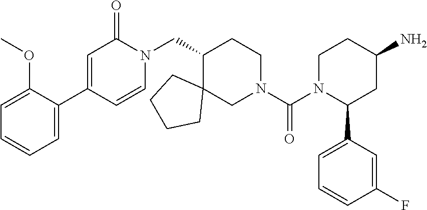 [SMILES: COc1ccccc1-c1ccn(C[C@@H]2CCN(C(=O)N3CC[C@@H](N)C[C@H]3c3cccc(F)c3)CC23CCCC3)c(=O)c1] | 1-(((R)-7-((2S,4R)-4- Amino-2-(3- fluorophenyl)piperidine- 1-carbonyl)-7- azaspiro[4.5]decan-10- yl)methyl)-4-(2- methoxyphenyl)pyridin- 2(1H)-one | 7.62 (d, J = 7.1 Hz, 1H), 7.41 (ddd, J = 8.6, 7.4, 1.7 Hz, 1H), 7.34 (dd, J = 7.6, 1.7 Hz, 1H), 7.27 (ddd, J = 8.9, 7.6, 6.2 Hz, 1H), 7.13 (dd, J = 8.4, 1.0 Hz, 1H), 7.03 (td, J = 7.4, 1.1 Hz, 2H), 6.99-6.93 (m, 2H), 6.46 (d, J = 1.9 Hz, 1H), 6.36 (dd, J = 7.0, 2.0 Hz, 1H), 4.06 (dd, J = 12.8, 3.0 Hz, 1H), 3.97 (dd, J = 11.8, 2.9 Hz, 1H), 3.90-3.83 (m, 1H), 3.80 (s, 3H), 3.70 (t, J = 11.9 Hz, 1H), 3.39 (d, J = 12.6 Hz, 1H), 3.26-3.18 (m, 2H), 2.90 (s, 1H), 2.81-2.68 (m, 2H), 2.68 2.60 (m, 1H, obscured by DMSO satellite), 1.89-1.72 (m, 4H), 1.71-1.12 (m, 12H). | (Method B) R_{T} = 0.96 min, m/z 573 [M + H]⁺ |
| 89 | 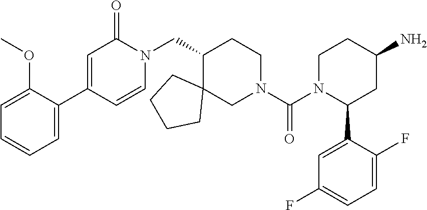 [SMILES: COc1ccccc1-c1ccn(C[C@@H]2CCN(C(=O)N3CC[C@@H](N)C[C@H]3c3cc(F)ccc3F)CC23CCCC3)c(=O)c1] | 1-(((R)-7-((2S,4R)-4- Amino-2-(2,5- difluorophenyl)piperidine- 1-carbonyl)-7- azaspiro[4.5]decan-10- yl)methyl)-4-(2- methoxyphenyl)pyridin- 2(1H)-one | 7.62 (d, J = 7.1 Hz, 1H), 7.41 (ddd, J = 8.8, 7.4, 1.7 Hz, 1H), 7.35 (dd, J = 7.6, 1.7 Hz, 1H), 7.17-7.11 (m, 2H), 7.06- 6.94 (m, 3H), 6.46 (d, J = 1.9 Hz, 1H), 6.37 (dd, J = 7.0, 2.0 Hz, 1H), 4.23 (d, J = 11.6 Hz, 1H), 4.12-4.03 (m, 1H), 3.87 (dd, J = 13.0, 4.9 Hz, 1H), 3.80 (s, 3H), 3.71 (s, 1H), 3.36 (s, 1H), 3.25 (dt, J = 12.1, 3.6 Hz, 1H), 2.92 (s, 1H), 2.77 (d, J = 12.8 Hz, 1H), 2.74-2.60 (m, 2H, obscured by DMSO satellite), 1.91-1.14 (m, 17H). | (Method B) R_{T} = 0.98 min, m/z 591 [M + H]⁺ |
| 90 | 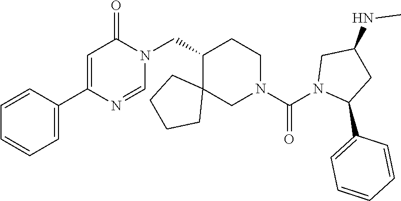 [SMILES: CN[C@H]1C[C@@H](c2ccccc2)N(C(=O)N2CC[C@@H](Cn3cnc(-c4ccccc4)cc3=O)C3(CCCC3)C2)C1] | 3-(((R)-7-((2S,4S)-4- (Methylamino)-2- phenylpyrrolidine-1- carbonyl)-7- azaspiro[4.5]decan-10- yl)methyl)-6- phenylpyrimidin-4(3H)- one | 7.74-7.68 (m, 3H), 7.52-7.43 (m, 3H), 7.30-7.20 (m, 4H), 7.20-7.15 (m, 1H), 6.65 (d, J = 2.1 Hz, 1H), 6.59-6.53 (m, 1H), 4.90 (dd, J = 11.0, 6.7 Hz, 1H), 4.11 (dd, J = 12.7, 3.0 Hz, 1H), 3.74 (d, J = 13.2 Hz, 1H), 3.63-3.52 (m, 2H), 3.36- 3.26 (m, 1H, obscured by HDO signal), 3.07 (s, 1H), 2.58 (d, J = 13.2 Hz, 1H), 2.55-2.44 (m, 2H, obscured by DMSO signal), 2.27 (s, 3H), 1.94 (ddd, J = 13.1, 8.0, 5.1 Hz, 1H), 1.84 (dd, J = 17.1, 9.3 Hz, 2H), 1.72-1.43 (m, 4H), 1.38 (q, J = | (Method B) R_{T} = 0.94 min, m/z 526 [M + H]⁺ |
| | | | 11.3 Hz, 1H), 1.26 (dt, J = 13.6, 8.8 Hz, | |
| | | | 4H), 1.20-1.11 (m, 1H). | |
| 91 | 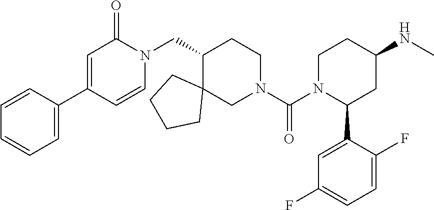 [SMILES: CN[C@@H]1CCN(C(=O)N2CC[C@@H](Cn3ccc(-c4ccccc4)cc3=O)C3(CCCC3)C2)[C@H](c2cc(F)ccc2F)C1] | 1-(((R)-7-((2S,4R)-2-(2,5- Difluorophenyl)-4- (methylamino)piperidine- 1-carbonyl)-7- azaspiro[4.5]decan-10- yl)methyl)-4- phenylpyridin-2(1H)-one | 8.55 (s, 1H), 8.10-8.02 (m, 2H), 7.49 (dd, J = 5.1, 1.9 Hz, 3H), 7.14 (td, J = 9.3, 4.6 Hz, 1H), 7.06-6.95 (m, 3H), 4.29- 4.23 (m, 1H), 4.11 (dd, J = 13.0, 3.1 Hz, 1H), 3.86 (dd, J = 11.1, 6.5 Hz, 1H), 3.73 (t, J = 12.1 Hz, 1H), 3.40 (d, J = 12.9 Hz, 1H), 3.29-3.23 (m, 1H), 2.86 (s, 1H), 2.75 (d, J = 12.9 Hz, 1H), 2.70-2.65 (m, 1H), 2.55-2.39 (m, 2H (obscured by DMSO signal)), 2.25 (s, 3H), 1.97-1.83 (m, 3H), 1.77 (s, 1H), 1.71-1.44 (m, 5H), 1.42-1.14 (m, 7H). | (Method B) R_{T} = 0.96 min, m/z 575 [M + H]⁺ |
| 92 | 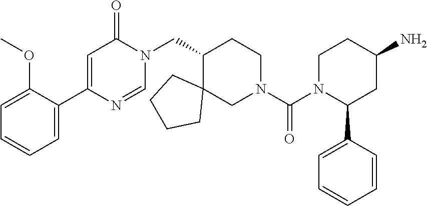 [SMILES: COc1ccccc1-c1cc(=O)n(C[C@@H]2CCN(C(=O)N3CC[C@@H](N)C[C@H]3c3ccccc3)CC23CCCC3)cn1] | 3-(((R)-7-((2S,4R)-4- Amino-2- phenylpiperidine-1- carbonyl)-7- azaspiro[4.5]decan-10- yl)methyl)-6-(2- methoxyphenyl)pyrimidin- 4(3H)-one | 8.52-8.47 (m, 1H), 7.95 (dd, J = 7.8, 1.8 Hz, 1H), 7.45 (ddd, J = 8.4, 7.3, 1.8 Hz, 1H), 7.17 (tdt, J = 15.6, 14.0, 6.7 Hz, 6H), 7.07 (td, J = 7.5, 1.1 Hz, 1H), 6.99 (d, J = 0.8 Hz, 1H), 4.08 (dd, J = 13.1, 3.0 Hz, 1H), 3.93 (dd, J = 11.7, 3.1 Hz, 1H), 3.88 (s, 4H), 3.69 (t, J = 11.9 Hz, 1H), 3.44 (d, J = 12.9 Hz, 1H), 3.22 (dt, J = 12.1, 3.7 Hz, 1H), 2.85 (s, 1H), 2.75-2.61 (m, 3H, obscured by DMSO satellite), 1.78 (s, 4H), 1.72-1.43 (m, 4H), 1.43-1.14 (m, 9H). | (Method B) R_{T} = 0.89 min, m/z 556 [M + H]⁺ |
| 93 | 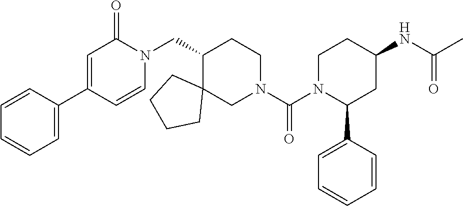 [SMILES: CC(=O)N[C@@H]1CCN(C(=O)N2CC[C@@H](Cn3ccc(-c4ccccc4)cc3=O)C3(CCCC3)C2)[C@H](c2ccccc2)C1] | N-((2S,4R)-1-((R)-10-((2- Oxo-4-phenylpyridin- 1(2H)-yl)methyl)-7- azaspiro[4.5]decane-7- carbonyl)-2- phenylpiperidin-4- yl)acetamide | 7.78-7.68 (m, 4H), 7.53-7.43 (m, 3H), 7.26-7.12 (m, 5H), 6.66 (d, J = 2.1 Hz, 1H), 6.58 (dd, J = 7.1, 2.1 Hz, 1H), 4.08 (dd, J = 12.7, 3.1 Hz, 1H), 3.98 (dd, J = 11.6, 3.2 Hz, 1H), 3.86 (d, J = 13.2 Hz, 1H), 3.70 (tdd, J = 11.6, 7.2, 3.5 Hz, 2H), 3.41 (d, J = 12.9 Hz, 1H), 3.29-3.20 (m, 1H), 2.88 (s, 1H), 2.70 (dd, J = 15.6, 12.2 Hz, 2H), 1.93-1.71 (m, 7H), 1.71-1.43 (m, 5H), 1.43-1.26 (m, 4H), 1.18 (dq, J = 14.1, 7.4 Hz, 2H). | (Method B) R_{T} = 1.26 min, m/z 567 [M + H]⁺ |
| 94 | 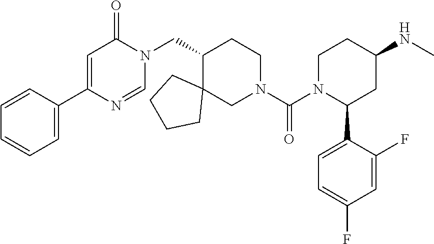 [SMILES: CN[C@@H]1CCN(C(=O)N2CC[C@@H](Cn3cnc(-c4ccccc4)cc3=O)C3(CCCC3)C2)[C@H](c2ccc(F)cc2F)C1] | 3-(((R)-7-((2S,4R)-2-(2,4- Difluorophenyl)-4- (methylamino)piperidine- 1-carbonyl)-7- azaspiro[4.5]decan-10- yl)methyl)-6- phenylpyrimidin-4(3H)- one | 8.55 (s, 1H), 8.10-8.04 (m, 2H), 7.49 (dd, J = 5.1, 1.9 Hz, 3H), 7.26 (td, J = 8.5, 6.7 Hz, 1H), 7.08 (ddd, J = 10.6, 9.4, 2.6 Hz, 1H), 6.99-6.91 (m, 2H), 4.23 (dd, J = 12.0, 3.0 Hz, 1H), 4.10 (dd, J = 13.0, 3.1 Hz, 1H), 3.84 (dd, J = 11.2, 6.6 Hz, 1H), 3.71 (t, J = 12.1 Hz, 1H), 3.38 (d, J = 13.0 Hz, 1H), 3.29-3.22 (m, 1H), 2.85 (s, 1H), 2.68 (d, J = 12.8 Hz, 2H), 2.44 (td, J = 10.7, 5.3 Hz, 1H), 2.24 (s, 3H), 1.96-1.81 (m, 3H), 1.81-1.43 (m, 6H), 1.34 (qd, J = 10.7, 9.5, 5.6 Hz, 3H), 1.21 (ddd, J = 18.8, 12.5, 5.7 Hz, 4H). | (Method B) R_{T} = 0.93 min, m/z 576 [M + H]⁺ |
| 95 | 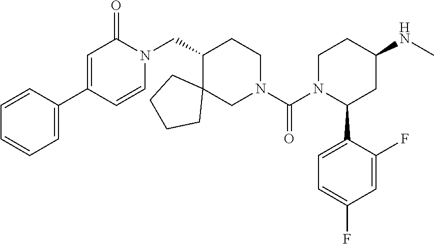 [SMILES: CN[C@@H]1CCN(C(=O)N2CC[C@@H](Cn3ccc(-c4ccccc4)cc3=O)C3(CCCC3)C2)[C@H](c2ccc(F)cc2F)C1] | 1-(((R)-7-((2S,4R)-2-(2,4- Difluorophenyl)-4- (methylamino)piperidine- 1-carbonyl)-7- azaspiro[4.5]decan-10- yl)methyl)-4- phenylpyridin-2(1H)-one | 7.77-7.67 (m, 3H), 7.52-7.43 (m, 3H), 7.26 (td, J = 8.5, 6.6 Hz, 1H), 7.08 (ddd, J = 10.6, 9.4, 2.6 Hz, 1H), 6.95 (td, J = 8.5, 2.6 Hz, 1H), 6.66 (d, J = 2.1 Hz, 1H), 6.57 (s, 1H), 4.23 (dd, J = 12.0, 3.0 Hz, 1H), 4.08 (dd, J = 12.7, 3.1 Hz, 1H), 3.83 (dd, J = 11.4, 6.8 Hz, 1H), 3.72 (t, J = 11.8 Hz, 1H), 3.36 (d, J = 14.0 Hz, 1H), 3.29- 3.22 (m, 1H), 2.88 (s, 1H), 2.76-2.65 (m, 2H), 2.43 (tt, J = 10.7, 4.0 Hz, 1H, obscured by DMSO signal), 2.24 (s, 3H), 1.96-1.41 (m, 9H), 1.34 (qd, J = 12.4, 11.9, 4.3 Hz, 3H), 1.27-1.08 (m, 4H). | (Method B) R_{T} = 0.96 min, m/z 575 [M + H]⁺ |
| 96 | 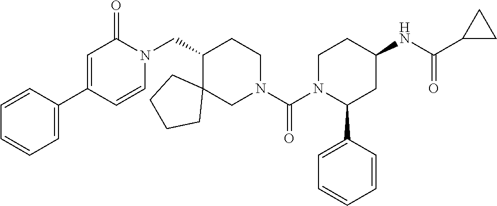 [SMILES: O=C(N[C@@H]1CCN(C(=O)N2CC[C@@H](Cn3ccc(-c4ccccc4)cc3=O)C3(CCCC3)C2)[C@H](c2ccccc2)C1)C1CC1] | N-((2S,4R)-1-((R)-10-((2- Oxo-4-phenylpyridin- 1(2H)-yl)methyl)-7- azaspiro[4.5]decane-7- carbonyl)-2- phenylpiperidin-4- yl)cyclopropanecarboxa mide | 7.97 (d, J = 7.6 Hz, 1H), 7.76-7.69 (m, 3H), 7.52-7.42 (m, 3H), 7.26-7.12 (m, 5H), 6.66 (d, J = 2.1 Hz, 1H), 6.58 (dd, J = 7.1, 2.1 Hz, 1H), 4.09 (dd, J = 12.8, 3.1 Hz, 1H), 3.97 (dd, J = 11.7, 3.2 Hz, 1H), 3.86 (dd, J = 11.3, 6.6 Hz, 1H), 3.73 (dtd, J = 12.2, 8.4, 8.0, 4.5 Hz, 2H), 3.42 (d, J = 12.9 Hz, 1H), 3.26 (dt, J = 12.3, 3.8 Hz, 1H), 2.88 (s, 1H), 2.70 (dd, J = 12.6, 10.0 Hz, 2H), 1.95-1.72 (m, 4H), 1.72-1.13 (m, 12H), 0.68-0.54 (m, 4H). | (Method B) R_{T} = 1.37 min, m/z 593 [M + H]⁺ |
| 97 | 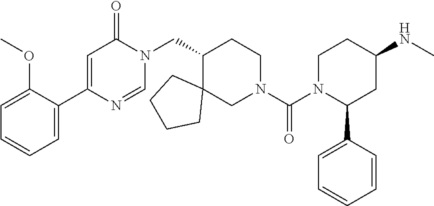 [SMILES: CN[C@@H]1CCN(C(=O)N2CC[C@@H](Cn3cnc(-c4ccccc4OC)cc3=O)C3(CCCC3)C2)[C@H](c2ccccc2)C1] | 6-(2-Methoxyphenyl)-3- (((R)-7-((2S,4R)-4- (methylamino)-2- phenylpiperidine-1- carbonyl)-7- azaspiro[4.5]decan-10- yl)methyl)pyrimidin- 4(3H)-one | 8.50 (s, 1H), 7.95 (dd, J = 7.8, 1.8 Hz, 1H), 7.45 (ddd, J = 8.8, 7.3, 1.8 Hz, 1H), 7.25-7.10 (m, 6H), 7.07 (td, J = 7.5, 1.1 Hz, 1H), 6.99 (s, 1H), 4.08 (dd, J = 13.0, 3.0 Hz, 1H), 3.95 (dd, J = 11.7, 3.1 Hz, 1H), 3.91-3.83 (m, 4H), 3.69 (t, J = 11.9 Hz, 1H), 3.44 (d, J = 12.9 Hz, 1H), 3.25 (dt, J = 12.0, 3.7 Hz, 1H), 2.85 (s, 1H), 2.71-2.62 (m, 2H, obscured by DMSO satellite), 2.43 (tt, J = 10.7, 4.0 Hz, 1H), 2.24 (s, 3H), 1.96-1.43 (m, 9H), 1.39- 1.13 (m, 7H). | (Method B) R_{T} = 0.91 min, m/z 570 [M + H]⁺ |
| 98 | 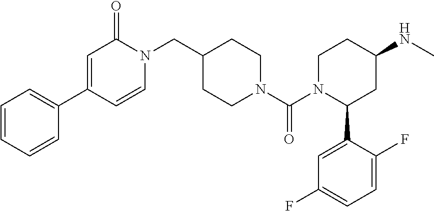 [SMILES: CN[C@@H]1CCN(C(=O)N2CCC(Cn3ccc(-c4ccccc4)cc3=O)CC2)[C@H](c2cc(F)ccc2F)C1] | 1-((1-((2S,4R)-2-(2,5- Difluorophenyl)-4- (methylamino)piperidine- 1-carbonyl)piperidin-4- yl)methyl)-4- phenylpyridin-2(1H)-one | 7.74-7.68 (m, 3H), 7.51-7.44 (m, 3H), 7.18-7.11 (m, 1H), 7.05-6.98 (m, 2H), 6.68-6.65 (m, 1H), 6.58-6.55 (m, 1H), 4.29-4.23 (m, 1H), 4.01-3.91 (m, 2H), 3.86-3.72 (m, 2H), 2.84-2.64 (m, 3H), 2.53-2.51 (m, 1H), 2.48-2.40 (m, 1H), 2.24 (s, 3H), 2.05-1.87 (m, 3H), 1.64-1.56 (m, 1H), 1.54-1.47 (m, 1H), 1.40-1.29 (m, 1H), 1.22-1.12 (m, 1H), 1.11-0.98 (m, 2H). [NH not observed] | (Method B) R_{T} = 0.84 min, m/z 521 [M + H]⁺ |
| 99 | 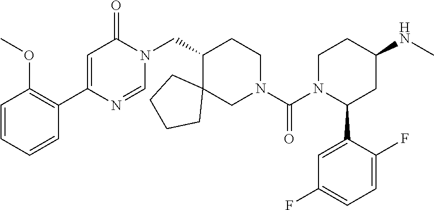 [SMILES: CN[C@@H]1CCN(C(=O)N2CC[C@@H](Cn3cnc(-c4ccccc4OC)cc3=O)C3(CCCC3)C2)[C@H](c2cc(F)ccc2F)C1] | 3-(((R)-7-((2S,4R)-2-(2,5- Difluorophenyl)-4- (methylamino)piperidine- 1-carbonyl)-7- azaspiro[4.5]decan-10- yl)methyl)-6-(2- methoxyphenyl)pyrimidin- 4(3H)-one | 8.50 (s, 1H), 7.97-7.93 (m, 1H), 7.48-7.43 (m, 1H), 7.19-7.10 (m, 2H), 7.09-6.96 (m, 4H), 4.28-4.23 (m, 1H), 4.12-4.06 (m, 1H), 3.90-3.83 (m, 4H), 3.76-3.66 (m, 1H), 3.44-3.36 (m, 1H), 3.29-3.24 (m, 1H), 2.87 (br s, 1H), 2.74 (d, 1H), 2.66 (t, 1H), 2.47-2.40 (m, 1H), 2.25 (s, 3H), 1.96- 1.83 (m, 3H), 1.83-1.71 (m, 1H), 1.71- 1.45 (m, 4H), 1.41-1.14 (m, 8H). | (Method B) R_{T} = 0.96 min, m/z 606 [M + H]⁺ |
| 100 | 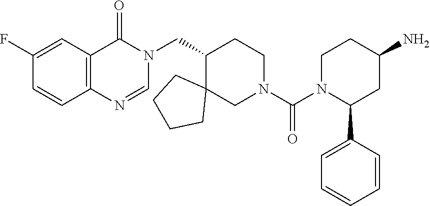 [SMILES: N[C@@H]1CCN(C(=O)N2CC[C@@H](Cn3cnc4ccc(F)cc4c3=O)C3(CCCC3)C2)[C@H](c2ccccc2)C1] | 3-(((R)-7-((2S,4R)-4- Amino-2- phenylpiperidine-1- carbonyl)-7- azaspiro[4.5]decan-10- yl)methyl)-6- fluoroquinazolin-4(3H)- one | 8.37 (s, 1H), 7.82 (dd, J = 8.7, 2.9 Hz, 1H), 7.80-7.69 (m, 2H), 7.24-7.15 (m, 4H), 7.15-7.10 (m, 1H), 4.15 (dd, J = 13.3, 3.0 Hz, 1H), 3.92 (dd, J = 11.7, 3.1 Hz, 1H), 3.87 (d, J = 13.3 Hz, 1H), 3.81- 3.73 (m, 1H), 3.45 (d, J = 12.9 Hz, 1H), 3.21 (dt, J = 12.2, 3.7 Hz, 1H), 2.81 (s, 1H), 2.73-2.60 (m, 3H), 1.91-1.74 (m, 4H), 1.72-1.43 (m, 6H), 1.40-1.26 (m, 4H), 1.26-1.15 (m, 3H). | (Method B) R_{T} = 0.86 min, m/z 518 [M + H]⁺ |
| 101 | 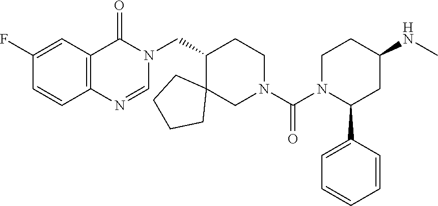 [SMILES: CN[C@@H]1CCN(C(=O)N2CC[C@@H](Cn3cnc4ccc(F)cc4c3=O)C3(CCCC3)C2)[C@H](c2ccccc2)C1] | 6-Fluoro-3-(((R)-7- ((2S,4R)-4- (methylamino)-2- phenylpiperidine-1- carbonyl)-7- azaspiro[4.5]decan-10- yl)methyl)quinazolin- 4(3H)-one | 8.36 (s, 1H), 7.84-7.69 (m, 3H), 7.24-7.10 (m, 5H), 4.18-4.12 (m, 1H), 3.98-3.92 (m, 1H), 3.89-3.83 (m, 1H), 3.81-3.73 (m, 1H), 3.48-3.40 (m, 1H), 3.28-3.21 (m, 2H), 2.87-2.75 (m, 1H), 2.70-2.61 (m, 2H), 2.28 (s, 3H), 1.99-1.74 (m, 4H), 1.72- 1.14 (m, 12H). | (Method B) R_{T} = 0.89 min, m/z 532 [M + H]⁺ |
| 102 | 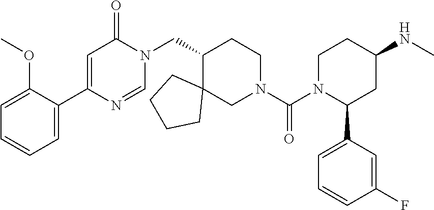 [SMILES: CN[C@@H]1CCN(C(=O)N2CC[C@@H](Cn3cnc(-c4ccccc4OC)cc3=O)C3(CCCC3)C2)[C@H](c2cccc(F)c2)C1] | 3-(((R)-7-((2S,4R)-2-(3- Fluorophenyl)-4- (methylamino)piperidine- 1-carbonyl)-7- azaspiro[4.5]decan-10- yl)methyl)-6-(2- methoxyphenyl)pyrimidin- 4(3H)-one | 8.50 (s, 1H), 7.95 (dd, J = 7.8, 1.8 Hz, 1H), 7.45 (ddd, J = 8.4, 7.3, 1.8 Hz, 1H), 7.29-7.23 (m, 1H), 7.16 (dd, J = 8.4, 1.0 Hz, 1H), 7.10-7.02 (m, 2H), 7.01-6.93 (m, 3H), 4.08 (dd, J = 13.0, 3.1 Hz, 1H), 3.99 (dd, J = 11.7, 3.0 Hz, 1H), 3.85 (s, 4H), 3.70 (t, J = 12.0 Hz, 1H), 3.42 (d, J = 12.9 Hz, 1H), 3.29-3.22 (m, 1H), 2.85 (s, 1H), 2.75-2.65 (m, 2H), 2.46-2.38 (m, 1H), 2.24 (s, 3H), 1.96-1.82 (m, 3H), 1.76 (d, J = 11.1 Hz, 1H), 1.71-1.44 (m, 5H), 1.32 (dq, J = 19.8, 11.6, 9.3 Hz, 4H), 1.24-1.12 (m, 3H). | (Method B) R_{T} = 0.97 min, m/z 588 [M + H]⁺ |
| 103 | 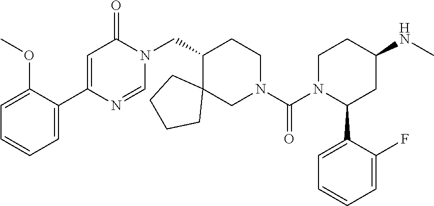 [SMILES: CN[C@@H]1CCN(C(=O)N2CC[C@@H](Cn3cnc(-c4ccccc4OC)cc3=O)C3(CCCC3)C2)[C@H](c2ccccc2F)C1] | 3-(((R)-7-((2S,4R)-2-(2- Fluorophenyl)-4- (methylamino)piperidine- 1-carbonyl)-7- azaspiro[4.5]decan-10- yl)methyl)-6-(2- methoxyphenyl)pyrimidin- 4(3H)-one | 8.50 (s, 1H), 7.95 (dd, J = 7.8, 1.8 Hz, 1H), 7.45 (ddd, J = 8.4, 7.3, 1.9 Hz, 1H), 7.24 (td, J = 7.5, 1.7 Hz, 1H), 7.21-7.13 (m, 2H), 7.09-7.02 (m, 3H), 6.99 (d, J = 0.9 Hz, 1H), 4.28 (dd, J = 11.9, 3.0 Hz, 1H), 4.08 (dd, J = 13.0, 3.1 Hz, 1H), 3.88 (s, 4H), 3.70 (t, J = 12.0 Hz, 1H), 3.40 (d, J = 12.9 Hz, 1H), 3.27 (t, J = 3.6 Hz, 1H), 2.87 (s, 1H), 2.74-2.64 (m, 2H), 2.43 (d, J = 10.9 Hz, 1H), 2.24 (s, 3H), 1.96-1.81 (m, 3H), 1.76 (s, 1H), 1.71-1.42 (m, 5H), 1.34 (qd, J = 12.1, 4.0 Hz, 3H), 1.30- 1.13 (m, 4H). | (Method B) R_{T} = 0.95 min, m/z 588 [M + H]⁺ |
| 104 | 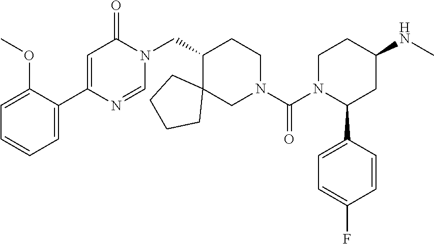 [SMILES: CN[C@@H]1CCN(C(=O)N2CC[C@@H](Cn3cnc(-c4ccccc4OC)cc3=O)C3(CCCC3)C2)[C@H](c2ccc(F)cc2)C1] | 3-(((R)-7-((2S,4R)-2-(4- Fluorophenyl)-4- (methylamino)piperidine- 1-carbonyl)-7- azaspiro[4.5]decan-10- yl)methyl)-6-(2- methoxyphenyl)pyrimidin- 4(3H)-one | 8.49 (s, 1H), 7.95 (dd, J = 7.8, 1.8 Hz, 1H), 7.45 (ddd, J = 8.4, 7.3, 1.8 Hz, 1H), 7.22 (dd, J = 8.6, 5.7 Hz, 2H), 7.16 (dd, J = 8.5, 1.1 Hz, 1H), 7.09-7.00 (m, 3H), 6.99 (d, J = 0.9 Hz, 1H), 4.08 (dd, J = 13.0, 3.0 Hz, 1H), 3.96 (dd, J = 11.7, 3.1 Hz, 1H), 3.88 (s, 4H), 3.69 (t, J = 12.0 Hz, 1H), 3.41 (d, J = 12.9 Hz, 1H), 3.27- 3.19 (m, 1H), 2.86 (s, 1H), 2.66 (dd, J = 9.2, 6.3 Hz, 2H), 2.46-2.39 (m, 1H), 2.24 (s, 3H), 1.96-1.79 (m, 3H), 1.75 (s, 1H), 1.71-1.42 (m, 5H), 1.33 (dq, J = 12.6, 7.7, 5.8 Hz, 3H), 1.27-1.12 (m, 4H). | (Method B) R_{T} = 0.96 min, m/z 588 [M + H]⁺ |
| 105 | 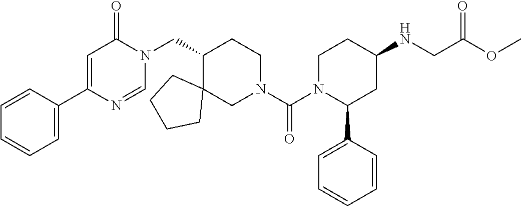 [SMILES: COC(=O)CN[C@@H]1CCN(C(=O)N2CC[C@@H](Cn3cnc(-c4ccccc4)cc3=O)C3(CCCC3)C2)[C@H](c2ccccc2)C1] | Methyl ((2S,4R)-1-((R)- 10-((6-oxo-4- phenylpyrimidin-1(6H)- yl)methyl)-7- azaspiro[4.5]decane-7- carbonyl)-2- phenylpiperidin-4- yl)glycinate | 8.55 (s, 1H), 8.09-8.04 (m, 2H), 7.49 (dd, J = 5.1, 1.9 Hz, 3H), 7.25-7.17 (m, 4H), 7.13 (ddt, J = 8.6, 6.1, 1.8 Hz, 1H), 6.97 (d, J = 0.8 Hz, 1H), 4.10 (dd, J = 13.2, 3.1 Hz, 1H), 3.93 (dd, J = 11.6, 3.1 Hz, 1H), 3.87 (d, J = 13.3 Hz, 1H), 3.70 (t, J = 12.0 Hz, 1H), 3.61 (s, 3H), 3.43 (d, J = 12.7 Hz, 1H), 3.37-3.32 (m, 2H), 3.27- 3.19 (m, 1H), 2.83 (s, 1H), 2.69-2.56 (m, 2H (obscured by DMSO satellite)), 1.98 (s, 1H), 1.85 (dd, J = 20.6, 10.6 Hz, 3H), 1.76 (s, 1H), 1.71-1.42 (m, 4H), 1.42- 1.13 (m, 8H). | (Method B) R_{T} = 0.98 min, m/z 598 [M + H]⁺ |
| 106 | 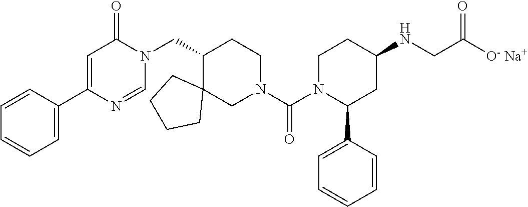 | Sodium ((2S,4R)-1-((R)- 10-((6-Oxo-4- phenylpyrimidin-1(6H)- yl)methyl)-7- azaspiro[4.5]decane-7- carbonyl)-2- phenylpiperidin-4- yl)glycinate | [Note: methanol-d4 instead of DMSO-de] 8.44 (s, 1H), 8.04-7.98 (m, 2H), 7.51- 7.46 (m, 3H), 7.29-7.22 (m, 4H), 7.19- 7.14 (m, 1H), 6.91 (s, 1H), 4.26 (dd, J = 13.2, 3.1 Hz, 1H), 4.07-3.98 (m, 2H), 3.81 (t, J = 11.9 Hz, 1H), 3.53 (d, J = 13.0 Hz, 1H), 3.46-3.40 (m, 1H), 3.27-3.24 (m, 2H), 3.04 (s, 1H), 2.91-2.76 (m, 3H), 2.07 (t, J = 14.0 Hz, 2H), 2.00-1.93 (m, 1H), 1.85 (s, 1H), 1.79-1.22 (m, 12H). [OH proton not observed]. | (Method B) R_{T} = 1.08 min, m/z 584 [M + H]⁺ |
| 107 | 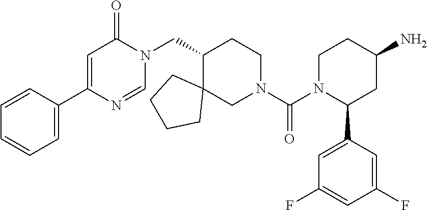 [SMILES: N[C@@H]1CCN(C(=O)N2CC[C@@H](Cn3cnc(-c4ccccc4)cc3=O)C3(CCCC3)C2)[C@H](c2cc(F)cc(F)c2)C1] | 3-(((R)-7-((2S,4R)-4- Amino-2-(3,5- difluorophenyl)piperidine- 1-carbonyl)-7- azaspiro[4.5]decan-10- yl)methyl)-6- phenylpyrimidin-4(3H)- one | 8.55 (s, 1H), 8.10-8.03 (m, 2H), 7.49 (dd, J = 5.1, 1.9 Hz, 3H), 6.97 (d, J = 4.4 Hz, 2H), 6.90-6.84 (m, 2H), 4.11 (dd, J = 13.0, 3.1 Hz, 1H), 3.98 (dd, J = 11.8, 3.0 Hz, 1H), 3.86 (d, J = 13.2 Hz, 1H), 3.77-3.67 (m, 1H), 3.41 (d, J = 12.8 Hz, 1H), 3.23 (dt, J = 12.2, 3.7 Hz, 1H), 2.85 (s, 1H), 2.77-2.65 (m, 2H), 2.62-2.57 (m, 1H), 1.91-1.73 (m, 4H), 1.73-1.43 (m, 6H), 1.43-1.14 (m, 7H). | (Method B) R_{T} = 1.03 min, m/z 562 [M + H]⁺ |
| 108 | 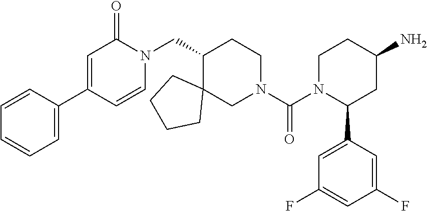 [SMILES: N[C@@H]1CCN(C(=O)N2CC[C@@H](Cn3ccc(-c4ccccc4)cc3=O)C3(CCCC3)C2)[C@H](c2cc(F)cc(F)c2)C1] | 1-(((R)-7-((2S,4R)-4- Amino-2-(3,5- difluorophenyl)piperidine- 1-carbonyl)-7- azaspiro[4.5]decan-10- yl)methyl)-4- phenylpyridin-2(1H)-one | 7.76-7.68 (m, 3H), 7.51-7.43 (m, 3H), 6.98 (tt, J = 9.2, 2.4 Hz, 1H), 6.91-6.84 (m, 2H), 6.66 (d, J = 2.1 Hz, 1H), 6.58 (dd, J = 7.1, 2.1 Hz, 1H), 4.09 (dd, J = 12.8, 3.2 Hz, 1H), 3.98 (dd, J = 11.8, 3.0 Hz, 1H), 3.86 (dd, J = 13.3, 4.9 Hz, 1H), 3.72 (s, 1H), 3.38 (d, J = 12.9 Hz, 1H), 3.23 (dt, J = 12.3, 3.6 Hz, 1H), 2.88 (s, 1H), 2.76 (d, J = 12.9 Hz, 1H), 2.73- 2.65 (m, 1H), 2.63-2.57 (m, 1H), 1.91- 1.71 (m, 4H), 1.71-1.42 (m, 6H), 1.42- 1.15 (m, 7H). | (Method B) R_{T} = 1.02 min, m/z 561 [M + H]⁺ |
| 109 | 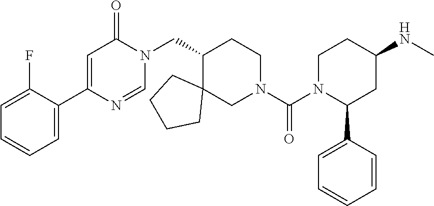 [SMILES: CN[C@@H]1CCN(C(=O)N2CC[C@@H](Cn3cnc(-c4ccccc4F)cc3=O)C3(CCCC3)C2)[C@H](c2ccccc2)C1] | 6-(2-Fluorophenyl)-3- (((R)-7-((2S,4R)-4- (methylamino)-2- phenylpiperidine-1- carbonyl)-7- azaspiro[4.5]decan-10- yl)methyl)pyrimidin- 4(3H)-one | 8.57 (s, 1H), 8.01 (td, J = 7.9, 1.9 Hz, 1H), 7.57-7.51 (m, 1H), 7.38-7.32 (m, 2H), 7.24-7.16 (m, 4H), 7.16-7.10 (m, 1H), 6.79 (d, J = 1.0 Hz, 1H), 4.10 (dd, J = 13.1, 3.0 Hz, 1H), 3.95 (dd, J = 11.7, 3.1 Hz, 1H), 3.88 (d, J = 13.3 Hz, 1H), 3.71 (t, J = 12.0 Hz, 1H), 3.44 (d, J = 12.8 Hz, 1H), 3.25 (dt, J = 12.2, 3.8 Hz, 1H), 2.84 (s, 1H), 2.71-2.64 (m, 2H), 2.44 (d, J = 10.8 Hz, 1H), 2.24 (s, 3H), 1.96-1.81 (m, 3H), 1.77 (s, 1H), 1.72-1.42 (m, 5H), 1.39-1.13 (m, 7H). | (Method B) R_{T} = 0.99 min, m/z 558 [M + H]⁺ |
| 110 | 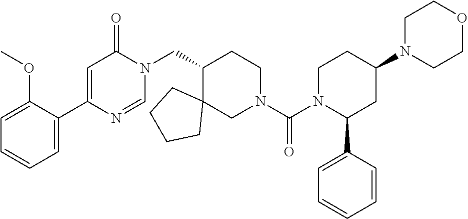 [SMILES: COc1ccccc1-c1cc(=O)n(C[C@@H]2CCN(C(=O)N3CC[C@@H](N4CCOCC4)C[C@H]3c3ccccc3)CC23CCCC3)cn1] | 6-(2-Methoxyphenyl)-3- (((R)-7-((2S,4R)-4- morpholino-2- phenylpiperidine-1- carbonyl)-7- azaspiro[4.5]decan-10- yl)methyl)pyrimidin- 4(3H)-one | 8.50 (s, 1H), 7.95 (dd, J = 7.8, 1.9 Hz, 1H), 7.48-7.42 (m, 1H), 7.24-7.18 (m, 4H), 7.18-7.11 (m, 2H), 7.07 (td, J = 7.5, 1.0 Hz, 1H), 6.99 (d, J = 0.8 Hz, 1H), 4.08 (dd, J = 13.0, 3.0 Hz, 1H), 3.96 (dd, J = 11.6, 3.1 Hz, 1H), 3.88 (s, 4H), 3.69 (t, J = 12.0 Hz, 1H), 3.51 (t, J = 4.6 Hz, 4H), 3.42 (s, 1H), 3.28 (d, J = 3.6 Hz, 1H), 2.86 (s, 1H), 2.70-2.65 (m, 2H, obscured by DMSO satellite), 2.46-2.41 (m, 3H (obscured by DMSO signal)), 2.41- 2.34 (m, 2H, obscured by DMSO satellite), 1.92-1.80 (m, 3H), 1.75 (d, J = | (Method B) R_{T} = 0.94 min, m/z 626 [M + H]⁺ |
| | | | 12.5 Hz, 1H), 1.70-1.42 (m, 5H), 1.42- | |
| | | | 1.13 (m, 6H). | |
| 111 | 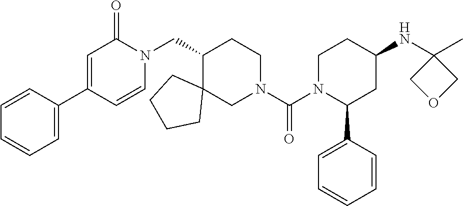 [SMILES: CC1(N[C@@H]2CCN(C(=O)N3CC[C@@H](Cn4ccc(-c5ccccc5)cc4=O)C4(CCCC4)C3)[C@H](c3ccccc3)C2)COC1] | 1-(((R)-7-((2S,4R)-4-((3- Methyloxetan-3- yl)amino)-2- phenylpiperidine-1- carbonyl)-7- azaspiro[4.5]decan-10- yl)methyl)-4- phenylpyridin-2(1H)-one | 7.76-7.69 (m, 3H), 7.51-7.44 (m, 3H), 7.20 (ddd, J = 14.2, 8.2, 6.6 Hz, 4H), 7.15- 7.11 (m, 1H), 6.66 (d, J = 2.1 Hz, 1H), 6.57 (dd, J = 7.1, 2.1 Hz, 1H), 4.38 (d, J = 5.5 Hz, 1H), 4.29 (d, J = 5.4 Hz, 1H), 4.17 (d, J = 5.4 Hz, 1H), 4.12 (d, J = 5.4 Hz, 1H), 4.08 (dd, J = 12.8, 3.1 Hz, 1H), 3.93 (dd, J = 11.8, 3.1 Hz, 1H), 3.87 (d, J = 13.2 Hz, 1H), 3.70 (t, J = 11.7 Hz, 1H), 3.42 (d, J = 12.7 Hz, 1H), 3.25-3.16 (m, 1H), 2.97-2.78 (m, 1H), 2.75-2.58 (m, 3H, obscured by DMSO satellite), 2.29 (s, 1H), 1.88-1.44 (m, 8H), 1.41 (s, 4H), 1.39-1.11 (m, 6H). | (Method B) R_{T} = 0.95 min, m/z 595 [M + H]⁺ |
| 112 | 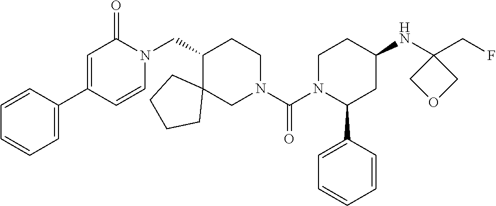 [SMILES: O=C(N1CC[C@@H](Cn2ccc(-c3ccccc3)cc2=O)C2(CCCC2)C1)N1CC[C@@H](NC2(CF)COC2)C[C@H]1c1ccccc1] | 1-(((R)-7-((2S,4R)-4-((3- (Fluoromethyl)oxetan-3- yl)amino)-2- phenylpiperidine-1- carbonyl)-7- azaspiro[4.5]decan-10- yl)methyl)-4- phenylpyridin-2(1H)-one | 7.75-7.68 (m, 3H), 7.51-7.43 (m, 3H), 7.20 (ddd, J = 14.1, 8.1, 6.5 Hz, 4H), 7.15- 7.11 (m, 1H), 6.66 (d, J = 2.1 Hz, 1H), 6.57 (dd, J = 7.1, 2.2 Hz, 1H), 4.69 (s, 1H), 4.60 (s, 1H), 4.39 (dd, J = 6.2, 3.8 Hz, 1H), 4.32 (dd, J = 6.2, 3.7 Hz, 2H), 4.29 (d, J = 6.0 Hz, 1H), 4.08 (dd, J = 12.8, 3.1 Hz, 1H), 3.93 (dd, J = 11.8, 3.1 Hz, 1H), 3.85 (s, 1H), 3.71 (t, J = 11.7 Hz, 1H), 3.42 (d, J = 12.9 Hz, 1H), 3.24- 3.17 (m, 1H), 2.84 (d, J = 21.4 Hz, 1H), 2.76 (dd, J = 7.5, 4.3 Hz, 1H), 2.70-2.59 (m, 2H, obscured by DMSO satellite), 1.89-1.12 (m, 16H). | (Method B) R_{T} = 1.01 min, m/z 613 [M + H]⁺ |
| 113 | 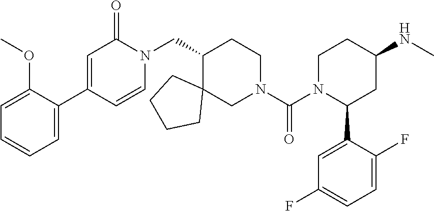 [SMILES: CN[C@@H]1CCN(C(=O)N2CC[C@@H](Cn3ccc(-c4ccccc4OC)cc3=O)C3(CCCC3)C2)[C@H](c2cc(F)ccc2F)C1] | 1-(((R)-7-((2S,4R)-2-(2,5- Difluorophenyl)-4- (methylamino)piperidine- 1-carbonyl)-7- azaspiro[4.5]decan-10- yl)methyl)-4-(2- methoxyphenyl)pyridin- 2(1H)-one | 7.62 (d, J = 7.0 Hz, 1H), 7.41 (ddd, J = 8.8, 7.4, 1.7 Hz, 1H), 7.34 (dd, J = 7.6, 1.7 Hz, 1H), 7.17-7.10 (m, 2H), 7.06- 6.96 (m, 3H), 6.46 (d, J = 1.9 Hz, 1H), 6.36 (dd, J = 7.0, 2.0 Hz, 1H), 4.26 (d, J = 11.9 Hz, 1H), 4.06 (dd, J = 13.2, 2.4 Hz, 1H), 3.86 (d, J = 13.3 Hz, 1H), 3.80 (s, 3H), 3.71 (s, 1H), 3.36 (d, J = 13.8 Hz, 1H), 3.29-3.24 (m, 1H), 2.91 (s, 1H), 2.77 (d, J = 12.9 Hz, 1H), 2.70-2.62 (m, 1H, obscured by DMSO satellite), 2.47- 2.39 (m, 1H), 2.25 (s, 3H), 1.92 (d, J = 12.7 Hz, 2H), 1.89-1.82 (m, 1H), 1.78 | (Method B) R_{T} = 0.96 min, m/z 605 [M + H]⁺ |
| | | | (s, 1H), 1.71-1.43 (m, 5H), 1.43-1.31 | |
| | | | (m, 3H), 1.31-1.13 (m, 4H). | |
| 114 | 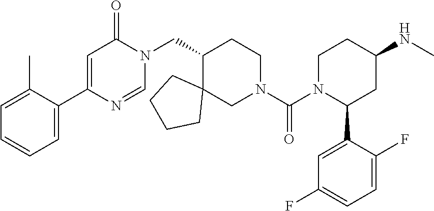 [SMILES: CN[C@@H]1CCN(C(=O)N2CC[C@@H](Cn3cnc(-c4ccccc4C)cc3=O)C3(CCCC3)C2)[C@H](c2cc(F)ccc2F)C1] | 3-(((R)-7-((2S,4R)-2-(2,5- Difluorophenyl)-4- (methylamino)piperidine- 1-carbonyl)-7- azaspiro[4.5]decan-10- yl)methyl)-6-(o- tolyl)pyrimidin-4(3H)-one | 8.52 (s, 1H), 7.41 (dd, J = 7.6, 1.4 Hz, 1H), 7.34 (td, J = 7.4, 1.5 Hz, 1H), 7.31- 7.24 (m, 2H), 7.14 (td, J = 9.3, 4.5 Hz, 1H), 7.06-6.96 (m, 2H), 6.46 (s, 1H), 4.26 (dt, J = 11.7, 2.3 Hz, 1H), 4.11 (dd, J = 13.0, 3.1 Hz, 1H), 3.92-3.82 (m, 1H), 3.74 (t, J = 12.2 Hz, 1H), 3.38 (d, J = 12.7 Hz, 1H), 3.28 (t, J = 4.2 Hz, 1H), 2.91 (s, 1H), 2.76 (d, J = 12.9 Hz, 1H), 2.71- 2.61 (m, 1H, obscured by DMSO satellite), 2.48-2.39 (m, 1H, obscured by DMSO signal), 2.36 (s, 3H, obscured by DMSO satellite), 2.25 (s, 3H), 1.96-1.84 | (Method B) R_{T} = 0.95 min, m/z 590 [M + H]⁺ |
| | | | (m, 3H), 1.77 (s, 1H), 1.71-1.43 (m, 5H), | |
| | | | 1.42-1.31 (m, 3H), 1.31-1.15 (m, 4H). | |
| 115 | 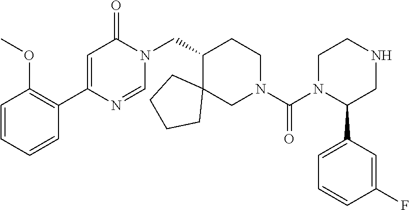 [SMILES: COc1ccccc1-c1cc(=O)n(C[C@@H]2CCN(C(=O)N3CCNC[C@H]3c3cccc(F)c3)CC23CCCC3)cn1] | 3-(((R)-7-((R)-2-(3- Fluorophenyl)piperazine- 1-carbonyl)-7- azaspiro[4.5]decan-10- yl)methyl)-6-(2- methoxyphenyl)pyrimidin- 4(3H)-one | 8.50 (s, 1H), 7.95 (dd, 1H), 7.45 (ddd, 1H), 7.31 (ddd, 1H), 7.16 (d, 1H), 7.14- 7.04 (m, 3H), 7.01 (td, 1H), 6.99 (s, 1H), 4.37 (t, 1H), 4.09 (dd, 1H), 3.88 (s, 3H), 3.75 (dt, 1H), 3.68 (t, 1H), 3.34 (s, 1H), 3.10-3.03 (m, 1H), 2.97-2.88 (m, 3H), 2.83-2.72 (m, 3H), 2.66 (d, 1H), 1.91-1.77 (m, 2H), 1.69-1.58 (m, 2H), 1.57-1.44 (m, 2H), 1.41-1.19 (m, 5H). [NH proton not observed]. | (Method B) R_{T} = 0.94 min, m/z 560 [M + H]⁺ |
| 116 | 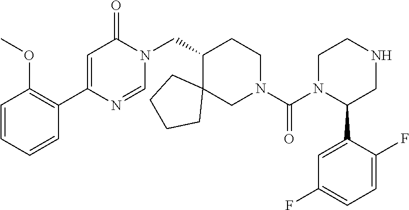 [SMILES: COc1ccccc1-c1cc(=O)n(C[C@@H]2CCN(C(=O)N3CCNC[C@H]3c3cc(F)ccc3F)CC23CCCC3)cn1] | 3-(((R)-7-((R)-2-(2,5- Difluorophenyl)piperazine- 1-carbonyl)-7- azaspiro[4.5]decan-10- yl)methyl)-6-(2- methoxyphenyl)pyrimidin- 4(3H)-one | 8.50 (s, 1H), 7.95 (dd, 1H), 7.48-7.41 (m, 1H), 7.21-7.11 (m, 3H), 7.10-7.02 (m, 2H), 6.99 (s, 1H), 4.44 (dd, 1H), 4.09 (dd, 1H), 3.88 (s, 3H), 3.81 (dt, 1H), 3.69 (t, 1H), 3.40-3.33 (m, 1H), 3.21-3.13 (m, 1H), 2.90 (dd, 1H), 2.87-2.74 (m, 4H), 2.73-2.65 (m, 2H), 1.90-1.76 (m, 2H), 1.69-1.58 (m, 2H), 1.57-1.44 (m, 2H), 1.40-1.18 (m, 5H). [NH proton not observed]. | (Method B) R_{T} = 0.93 min, m/z 578 [M + H]⁺ |
| 117 | 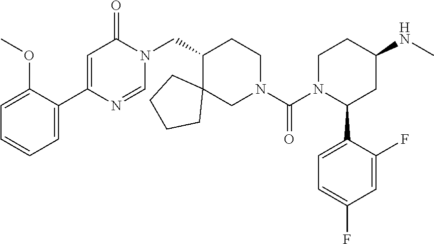 [SMILES: CN[C@@H]1CCN(C(=O)N2CC[C@@H](Cn3cnc(-c4ccccc4OC)cc3=O)C3(CCCC3)C2)[C@H](c2ccc(F)cc2F)C1] | 3-(((R)-7-((2S,4R)-2-(2,4- Difluorophenyl)-4- (methylamino)piperidine- 1-carbonyl)-7- azaspiro[4.5]decan-10- yl)methyl)-6-(2- methoxyphenyl)pyrimidin- 4(3H)-one | 8.50 (s, 1H), 7.95 (dd, J = 7.8, 1.8 Hz, 1H), 7.45 (ddd, J = 8.8, 7.3, 1.9 Hz, 1H), 7.26 (td, J = 8.5, 6.7 Hz, 1H), 7.16 (dd, J = 8.4, 1.0 Hz, 1H), 7.12-7.04 (m, 2H), 6.99 (d, J = 0.8 Hz, 1H), 6.95 (td, J = 8.5, 2.6 Hz, 1H), 4.24 (dd, J = 12.0, 3.1 Hz, 1H), 4.08 (dd, J = 13.0, 3.0 Hz, 1H), 3.92- 3.80 (m, 4H), 3.70 (t, J = 12.0 Hz, 1H), 3.38 (d, J = 13.0 Hz, 1H), 3.29-3.22 (m, 1H), 2.87 (s, 1H), 2.73-2.65 (m, 2H, obscured by DMSO satellite), 2.44 (m, 2H, obscured by DMSO signal), 2.25 (s, 3H), 1.96-1.81 (m, 3H), 1.75 (s, 1H), 1.71-1.58 (m, 2H), 1.51 (dtd, J = 23.2, 13.3, 11.5, 7.5 Hz, 2H), 1.34 (dt, J = 12.3, 8.8 Hz, 3H), 1.28-1.13 (m, 4H). | (Method B) R_{T} = 0.93 min, m/z 606 [M + H]⁺ |
| 118 | 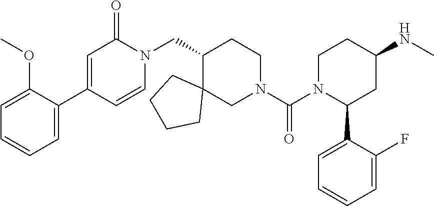 [SMILES: CN[C@@H]1CCN(C(=O)N2CC[C@@H](Cn3ccc(-c4ccccc4OC)cc3=O)C3(CCCC3)C2)[C@H](c2ccccc2F)C1] | 1-(((R)-7-((2S,4R)-2-(2- Fluorophenyl)-4- (methylamino)piperidine- 1-carbonyl)-7- azaspiro[4.5]decan-10- yl)methyl)-4-(2- methoxyphenyl)pyridin- 2(1H)-one | 7.62 (d, J = 7.1 Hz, 1H), 7.41 (ddd, J = 8.7, 7.4, 1.7 Hz, 1H), 7.35 (dd, J = 7.6, 1.7 Hz, 1H), 7.24 (td, J = 7.5, 1.7 Hz, 1H), 7.22-7.15 (m, 1H), 7.13 (dd, J = 8.4, 1.0 Hz, 1H), 7.10-7.01 (m, 3H), 6.46 (d, J = 1.9 Hz, 1H), 6.36 (dd, J = 7.0, 2.0 Hz, 1H), 4.28 (dd, J = 11.9, 3.0 Hz, 1H), 4.06 (dd, J = 12.7, 3.0 Hz, 1H), 3.90-3.83 (m, 1H), 3.80 (s, 3H), 3.70 (t, J = 11.7 Hz, 1H), 3.38 (d, J = 12.8 Hz, 1H), 3.27 (d, J = 3.6 Hz, 1H), 2.91 (s, 1H), 2.75-2.65 (m, 2H, obscured by DMSO satellite), 2.47-2.39 (m, 1H, obscured by DMSO | (Method B) R_{T} = 0.95 min, m/z 587 [M + H]⁺ |
| | | | signal), 2.25 (s, 3H), 1.92 (td, J = 13.0, | |
| | | | 11.0, 3.2 Hz, 2H), 1.84 (td, J = 10.2, 5.0 Hz, | |
| | | | 1H), 1.77 (s, 1H), 1.71-1.42 (m, 5H), | |
| | | | 1.42-1.11 (m, 7H). | |
| 119 | 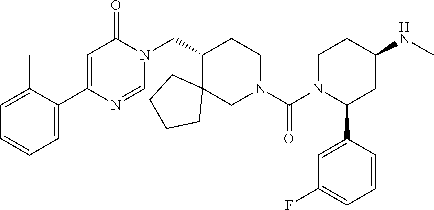 [SMILES: CN[C@@H]1CCN(C(=O)N2CC[C@@H](Cn3cnc(-c4ccccc4C)cc3=O)C3(CCCC3)C2)[C@H](c2cccc(F)c2)C1] | 3-(((R)-7-((2S,4R)-2-(3- Fluorophenyl)-4- (methylamino)piperidine- 1-carbonyl)-7- azaspiro[4.5]decan-10- yl)methyl)-6-(0- tolyl)pyrimidin-4(3H)-one | 8.52 (s, 1H), 7.43-7.39 (m, 1H), 7.34 (td, J = 7.4, 1.5 Hz, 1H), 7.27 (ddd, J = 12.2, 7.9, 6.2 Hz, 3H), 7.05 (d, J = 7.7 Hz, 1H), 7.00-6.92 (m, 2H), 6.46 (s, 1H), 4.11 (dd, J = 12.9, 3.0 Hz, 1H), 3.99 (dd, J = 11.7, 3.0 Hz, 1H), 3.88 (dt, J = 13.3, 4.4 Hz, 1H), 3.73 (t, J = 11.9 Hz, 1H), 3.41 (d, J = 12.9 Hz, 1H), 3.27 (dt, J = 12.2, 3.4 Hz, 1H), 2.89 (s, 1H), 2.76-2.65 (m, 2H, obscured by DMSO satellite), 2.47-2.38 (m, 1H, obscured by DMSO signal), 2.36 (s, 3H, obscured by DMSO satellite), 2.24 (s, 3H), 1.96-1.82 (m, 3H), 1.76 (s, 1H), 1.70-1.43 (m, 5H), 1.40-1.13 (m, 7H). | (Method B) R_{T} = 0.94 min, m/z 572 [M + H]⁺ |
| 120 | 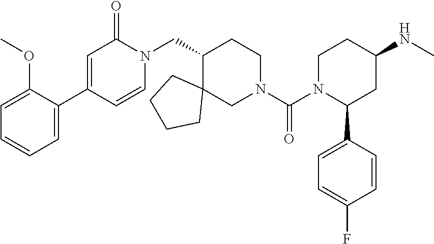 [SMILES: CN[C@@H]1CCN(C(=O)N2CC[C@@H](Cn3ccc(-c4ccccc4OC)cc3=O)C3(CCCC3)C2)[C@H](c2ccc(F)cc2)C1] | 1-(((R)-7-((2S,4R)-2-(4- Fluorophenyl)-4- (methylamino)piperidine- 1-carbonyl)-7- azaspiro[4.5]decan-10- yl)methyl)-4-(2- methoxyphenyl)pyridin- 2(1H)-one | 7.61 (d, J = 7.1 Hz, 1H), 7.41 (ddd, J = 8.7, 7.5, 1.8 Hz, 1H), 7.34 (dd, J = 7.6, 1.7 Hz, 1H), 7.22 (dd, J = 8.6, 5.7 Hz, 2H), 7.13 (d, J = 8.3 Hz, 1H), 7.06-6.99 (m, 3H), 6.45 (d, J = 2.0 Hz, 1H), 6.36 (dd, J = 7.0, 2.0 Hz, 1H), 4.05 (dd, J = 12.6, 3.0 Hz, 1H), 3.96 (dd, J = 11.8, 3.1 Hz, 1H), 3.85 (d, J = 13.2 Hz, 1H), 3.80 (s, 3H), 3.69 (t, J = 11.8 Hz, 1H), 3.38 (d, J = 12.9 Hz, 1H), 3.24 (dd, J = 12.2, 3.9 Hz, 1H), 2.89 (s, 1H), 2.73-2.65 (m, 2H, obscured by DMSO satellite), 2.42 (td, J = 10.4, 5.0 Hz, 1H, obscured by DMSO signal), 2.24 (s, 3H), 1.96-1.80 (m, 3H), 1.76 (s, 1H), 1.71-1.40 (m, 5H), 1.33 (tt, | (Method B) R_{T} = 0.96 min, m/z 587 [M + H]⁺ |
| | | | J = 10.6, 5.9 Hz, 3H), 1.18 (tq, J = 19.7, | |
| | | | 7.1 Hz, 4H). | |
| 121 | 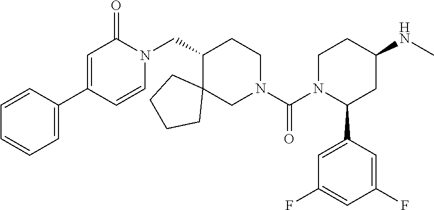 [SMILES: CN[C@@H]1CCN(C(=O)N2CC[C@@H](Cn3ccc(-c4ccccc4)cc3=O)C3(CCCC3)C2)[C@H](c2cc(F)cc(F)c2)C1] | 1-(((R)-7-((2S,4R)-2-(3,5- Difluorophenyl)-4- (methylamino)piperidine- 1-carbonyl)-7- azaspiro[4.5]decan-10- yl)methyl)-4- phenylpyridin-2(1H)-one | 7.76-7.68 (m, 3H), 7.52-7.43 (m, 3H), 6.98 (tt, J = 9.3, 2.4 Hz, 1H), 6.92-6.85 (m, 2H), 6.66 (d, J = 2.1 Hz, 1H), 6.58 (dd, J = 7.1, 2.1 Hz, 1H), 4.09 (dd, J = 13.0, 3.1 Hz, 1H), 4.01 (dd, J = 11.6, 3.1 Hz, 1H), 3.89-3.81 (m, 1H), 3.72 (t, J = 11.8 Hz, 1H), 3.38 (d, J = 12.7 Hz, 1H), 3.29-3.22 (m, 1H), 2.88 (s, 1H), 2.76 (d, J = 12.9 Hz, 1H), 2.68-2.60 (m, 1H, obscured by DMSO satellite), 2.46-2.38 (m, 1H), 2.24 (s, 3H), 1.89 (dt, J = 22.1, 12.5 Hz, 3H), 1.78 (s, 1H), 1.65 (s, 2H), 1.60-1.42 (m, 3H), 1.42-1.11 (m, 7H). | (Method B) R_{T} = 0.97 min, m/z 575 [M + H]⁺ |
| 122 | 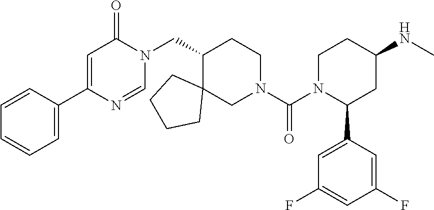 [SMILES: CN[C@@H]1CCN(C(=O)N2CC[C@@H](Cn3cnc(-c4ccccc4)cc3=O)C3(CCCC3)C2)[C@H](c2cc(F)cc(F)c2)C1] | 3-(((R)-7-((2S,4R)-2-(3,5- Difluorophenyl)-4- (methylamino)piperidine- 1-carbonyl)-7- azaspiro[4.5]decan-10- yl)methyl)-6- phenylpyrimidin-4(3H)- one | 8.55 (s, 1H), 8.10-8.03 (m, 2H), 7.49 (dd, J = 5.1, 1.9 Hz, 3H), 7.02-6.94 (m, 2H), 6.92-6.85 (m, 2H), 4.11 (dd, J = 13.1, 3.1 Hz, 1H), 4.01 (dd, J = 11.7, 3.1 Hz, 1H), 3.85 (d, J = 13.1 Hz, 1H), 3.72 (t, J = 12.1 Hz, 1H), 3.41 (d, J = 12.9 Hz, 1H), 3.29-3.22 (m, 1H), 2.85 (s, 1H), 2.74 (d, J = 12.8 Hz, 1H), 2.69 2.60 (m, 1H, obscured by DMSO satellite), 2.43 (td, J = 10.6, 5.2 Hz, 1H), 2.24 (s, 3H), 1.97-1.83 (m, 3H), 1.78 (s, 1H), 1.71- 1.43 (m, 5H), 1.42-1.11 (m, 7H) | (Method B) R_{T} = 0.96 min, m/z 576 [M + H]⁺ |
| 123 | 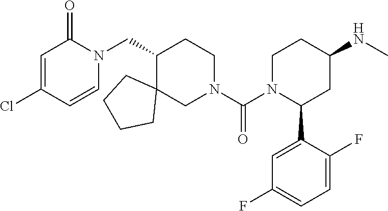 [SMILES: CN[C@@H]1CCN(C(=O)N2CC[C@@H](Cn3ccc(Cl)cc3=O)C3(CCCC3)C2)[C@H](c2cc(F)ccc2F)C1] | 4-Chloro-1-(((R)-7- ((2S,4R)-2-(2,5- difluorophenyl)-4- (methylamino)piperidine- 1-carbonyl)-7- azaspiro[4.5]decan-10- yl)methyl)pyridin-2(1H)- one | 7.73 (d, 1H), 7.13 (td, 1H), 7.05-6.93 (m, 2H), 6.52 (d, 1H), 6.35 (dd, 1H), 4.25 (d, 1H), 4.04 (dd, 1H), 3.82 (dt, 1H), 3.68 (t, 1H), 3.43-3.35 (m, 1H), 3.28-3.23 (m, 1H), 2.86 (br s, 1H), 2.73 (d, 1H), 2.69- 2.61 (m, 1H), 2.48-2.40 (m, 2H), 2.25 (s, 3H), 1.96-1.87 (m, 2H), 1.84-1.68 (m, 2H), 1.67-1.50 (m, 3H), 1.49-1.42 (m, 1H), 1.40-1.29 (m, 2H), 1.28-1.22 (m, 2H), 1.21-1.11 (m, 3H). | (Method B) R_{T} = 0.92 min, m/z 533 [M + H]⁺ |
| 124 | 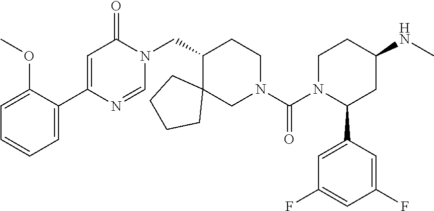 [SMILES: CN[C@@H]1CCN(C(=O)N2CC[C@@H](Cn3cnc(-c4ccccc4OC)cc3=O)C3(CCCC3)C2)[C@H](c2cc(F)cc(F)c2)C1] | 3-(((R)-7-((2S,4R)-2-(3,5- Difluorophenyl)-4- (methylamino)piperidine- 1-carbonyl)-7- azaspiro[4.5]decan-10- yl)methyl)-6-(2- methoxyphenyl)pyrimidin- 4(3H)-one | 8.50 (s, 1H), 7.95 (dd, J = 7.8, 1.8 Hz, 1H), 7.45 (ddd, J = 8.6, 7.3, 1.8 Hz, 1H), 7.16 (dd, J = 8.5, 1.0 Hz, 1H), 7.07 (td, J = 7.6, 1.1 Hz, 1H), 7.01-6.95 (m, 2H), 6.92-6.85 (m, 2H), 4.09 (dd, J = 13.0, 3.0 Hz, 1H), 4.01 (dd, J = 11.8, 3.1 Hz, 1H), 3.92-3.81 (m, 4H), 3.71 (t, J = 12.0 Hz, 1H), 3.40 (d, J = 12.9 Hz, 1H), 3.29- 3.21 (m, 1H), 2.86 (s, 1H), 2.74 (d, J = 12.9 Hz, 1H), 2.67 (s, 1H, obscured by DMSO satellite), 2.47-2.38 (m, 1H, obscured by DMSO signal), 2.24 (s, 3H), 1.98-1.83 (m, 3H), 1.78 (s, 1H), 1.65 (s, | (Method B) R_{T} = 1.00 min, m/z 606 [M + H]⁺ |
| | | | 2H), 1.61-1.44 (m, 3H), 1.41-1.11 (m, | |
| | | | 7H). | |
| 125 | 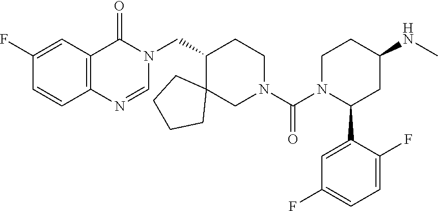 [SMILES: CN[C@@H]1CCN(C(=O)N2CC[C@@H](Cn3cnc4ccc(F)cc4c3=O)C3(CCCC3)C2)[C@H](c2cc(F)ccc2F)C1] | 3-(((R)-7-((2S,4R)-2-(2,5- Difluorophenyl)-4- (methylamino)piperidine- 1-carbonyl)-7- azaspiro[4.5]decan-10- yl)methyl)-6- fluoroquinazolin-4(3H)- one | 8.37 (s, 1H), 7.83 (dd, 1H), 7.80-7.68 (m, 2H), 7.13 (td, 1H), 7.05-6.94 (m, 2H), 4.25 (d, 1H), 4.16 (d, 1H), 3.90-3.72 (m, 2H), 3.40 (d, 1H), 3.29-3.26 (m, 1H), 2.83 (br s, 1H), 2.74 (d, 1H), 2.66 (t, 1H), 2.46- 2.39 (m, 1H), 2.25 (s, 3H), 1.97-1.85 (m, 3H), 1.85-1.44 (m, 6H), 1.42-1.27 (m, 4H), 1.26-1.14 (3H). | (Method B) R_{T} = 0.88 min, m/z 568 [M + H]⁺ |
| 126 | 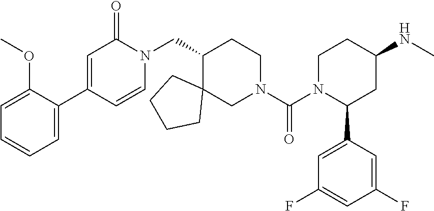 [SMILES: CN[C@@H]1CCN(C(=O)N2CC[C@@H](Cn3ccc(-c4ccccc4OC)cc3=O)C3(CCCC3)C2)[C@H](c2cc(F)cc(F)c2)C1] | 1-(((R)-7-((2S,4R)-2-(3,5- Difluorophenyl)-4- (methylamino)piperidine- 1-carbonyl)-7- azaspiro[4.5]decan-10- yl)methyl)-4-(2- methoxyphenyl)pyridin- 2(1H)-one | 7.62 (d, J = 7.1 Hz, 1H), 7.41 (ddd, J = 8.7, 7.4, 1.8 Hz, 1H), 7.34 (dd, J = 7.6, 1.7 Hz, 1H), 7.13 (dd, J = 8.4, 1.0 Hz, 1H), 7.03 (td, J = 7.5, 1.0 Hz, 1H), 6.98 (tt, J = 9.3, 2.4 Hz, 1H), 6.92-6.85 (m, 2H), 6.46 (d, J = 1.9 Hz, 1H), 6.36 (d, J = 2.0 Hz, 1H), 4.06 (dd, J = 13.0, 3.0 Hz, 1H), 4.01 (dd, J = 11.7, 3.1 Hz, 1H), 3.89- 3.82 (m, 1H), 3.80 (s, 3H), 3.72 (d, J = 10.7 Hz, 1H), 3.37 (d, J = 13.1 Hz, 1H), 3.29-3.23 (m, 1H), 2.89 (s, 1H), 2.76 (d, J = 12.9 Hz, 1H), 2.69-2.64 (m, 1H), 2.43 (td, J = 10.7, 5.3 Hz, 1H), 2.24 (s, | (Method B) R_{T} = 0.99 min, m/z 605 [M + H]⁺ |
| | | | 3H), 1.88 (ddd, J = 38.6, 26.9, 19.2 Hz, | |
| | | | 4H), 1.71-1.43 (m, 5H), 1.43-1.11 (m, | |
| | | | 7H). | |
| 127 | 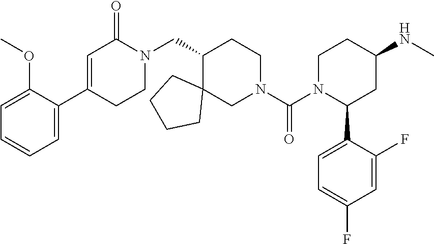 [SMILES: CN[C@@H]1CCN(C(=O)N2CC[C@@H](CN3CCC(c4ccccc4OC)=CC3=O)C3(CCCC3)C2)[C@H](c2ccc(F)cc2F)C1] | 1-(((R)-7-((2S,4R)-2-(2,4- Difluorophenyl)-4- (methylamino)piperidine- 1-carbonyl)-7- azaspiro[4.5]decan-10- yl)methyl)-4-(2- methoxyphenyl)-5,6- dihydropyridin-2(1H)-one and 1-(((S)-7-((2S,4R)-2-(2,4- Difluorophenyl)-4- (methylamino)piperidine- 1-carbonyl)-7- azaspiro[4.5]decan-10- yl)methyl)-4-(2- | 7.36 (ddt, J = 8.6, 7.3, 1.4 Hz, 1H), 7.29 (ddt, J = 6.6, 4.8, 2.7 Hz, 2H), 7.13-7.05 (m, 2H), 7.02-6.93 (m, 2H), 5.95 (t, J = 1.6 Hz, 1H), 4.21 (ddd, J = 17.7, 11.9, 2.9 Hz, 1H), 3.81 (s, 3H), 3.61-3.37 (m, 3H), 3.29-3.20 (m, 2H), 3.20-3.03 (m, 2H), 2.79-2.61 (m, 3H), 2.60-2.53 (m, 1H), 2.46-2.38 (m, 1H), 2.25 (s, 3H), 1.97-1.86 (m, 2H), 1.75-1.05 (m, 14H), 0.97 (s, 1H) | (Method B) RT = 1.02 min, m/z 607 [M+H]+ |
| | | methoxyphenyl)-5,6- | | |
| | | dihydropyridin-2(1H)-one | | |
| | and | | | |
| | 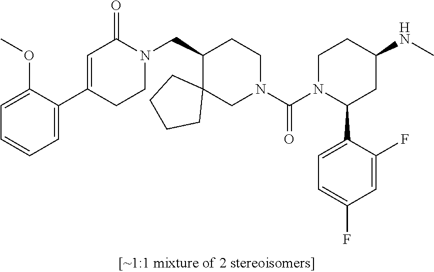 [SMILES: CN[C@@H]1CCN(C(=O)N2CC[C@H](CN3CCC(c4ccccc4OC)=CC3=O)C3(CCCC3)C2)[C@H](c2ccc(F)cc2F)C1] | | | |
| 128 | 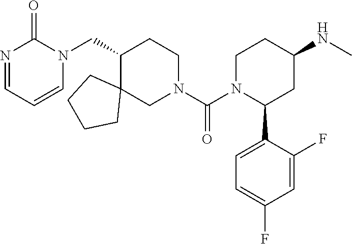 [SMILES: CN[C@@H]1CCN(C(=O)N2CC[C@@H](Cn3cccnc3=O)C3(CCCC3)C2)[C@H](c2ccc(F)cc2F)C1] | 1-(((R)-7-((2S,4R)-2-(2,4- Difluorophenyl)-4- (methylamino)piperidine- 1-carbonyl)-7- azaspiro[4.5]decan-10- yl)methyl)pyrimidin- 2(1H)-one | 8.53 (dd, J = 4.1, 2.8 Hz, 1H), 8.15 (dd, J = 6.5, 2.8 Hz, 1H), 7.25 (td, J = 8.5, 6.7 Hz, 1H), 7.08 (ddd, J = 10.9, 9.5, 2.6 Hz, 1H), 6.95 (td, J = 8.4, 2.5 Hz, 1H), 6.41 (dd, J = 6.4, 4.2 Hz, 1H), 4.23 (dd, J = 12.0, 3.0 Hz, 1H), 4.05 (dd, J = 12.8, 3.0 Hz, 1H), 3.82 (dt, J = 13.1, 4.4 Hz, 1H), 3.60 (t, J = 11.8 Hz, 1H), 3.41-3.33 (m, 1H), 3.26 (dt, J = 12.1, 3.9 Hz, 1H), 2.85 (s, 1H), 2.72-2.62 (m, 2H, obscured by DMSO satellite), 2.42 (td, J = 10.7, 5.2 Hz, 1H, obscured by DMSO signal), 2.24 (s, 3H), 1.96-1.78 (m, 3H), 1.72 (s, 1H), 1.68-1.39 (m, 5H), 1.39-1.09 (m, 7H). | (Method B) RT = 0.70 min, m/z 500 [M+H]+ |
| 129 | 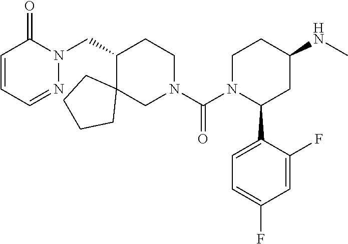 [SMILES: CN[C@@H]1CCN(C(=O)N2CC[C@@H](Cn3ncccc3=O)C3(CCCC3)C2)[C@H](c2ccc(F)cc2F)C1] | 2-(((R)-7-((2S,4R)-2-(2,4- Difluorophenyl)-4- (methylamino)piperidine- 1-carbonyl)-7- azaspiro[4.5]decan-10- yl)methyl)pyridazin- 3(2H)-one | 7.92 (dd, J = 3.8, 1.6 Hz, 1H), 7.40 (dd, J = 9.4, 3.9 Hz, 1H), 7.25 (td, J = 8.4, 6.6 Hz, 1H), 7.08 (ddd, J = 10.6, 9.4, 2.6 Hz, 1H), 6.94 (ddd, J = 11.1, 8.9, 2.1 Hz, 2H), 4.22 (dd, J = 12.0, 3.0 Hz, 1H), 4.10- 3.98 (m, 2H), 3.76 (dt, J = 13.5, 4.7 Hz, 1H), 3.26 (dt, J = 12.2, 3.8 Hz, 2H), 2.98 (s, 1H), 2.75 (d, J = 12.9 Hz, 1H), 2.69- 2.61 (m, 1H), 2.46-2.38 (m, 1H), 2.24 (s, 3H), 1.96-1.84 (m, 3H), 1.69-1.37 (m, 7H), 1.37-1.10 (m, 6H). | (Method B) R_{T} = 0.84 min, m/z 500 [M + H]⁺ |
| 130 | 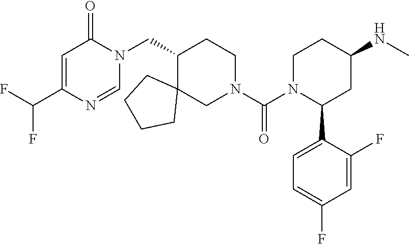 [SMILES: CN[C@@H]1CCN(C(=O)N2CC[C@@H](Cn3cnc(C(F)F)cc3=O)C3(CCCC3)C2)[C@H](c2ccc(F)cc2F)C1] | 6-(Difluoromethyl)-3- (((R)-7-((2S,4R)-2-(2,4- difluorophenyl)-4- (methylamino)piperidine- 1-carbonyl)-7- azaspiro[4.5]decan-10- yl)methyl)pyrimidin- 4(3H)-one | 8.58 (s, 1H), 7.25 (td, J = 8.4, 6.6 Hz, 1H), 7.08 (ddd, J = 10.7, 9.3, 2.6 Hz, 1H), 6.95 (td, J = 8.5, 2.6 Hz, 1H), 6.87-6.61 (m, 2H), 4.23 (dd, J = 12.0, 3.0 Hz, 1H), 4.08 (dd, J = 13.1, 3.0 Hz, 1H), 3.83 (dt, J = 13.5, 4.6 Hz, 1H), 3.71 (t, J = 12.0 Hz, 1H), 3.37 (d, J = 12.9 Hz, 1H), 3.33- 3.21 (m, 2H, obscured by HDO signal), 2.83 (s, 1H), 2.71-2.64 (m, 2H, obscured by DMSO satellite), 2.46-2.41 (m, 1H, obscured by DMSO signal), 2.25 (s, 3H), 1.96-1.86 (m, 2H), 1.81 (tt, J = 10.7, 3.5 Hz, 1H), 1.77-1.40 (m, 5H), 1.40-1.10 (m, 7H). | (Method B) R_{T} = 0.80 min, m/z 550 [M + H]⁺ |
| 131 | 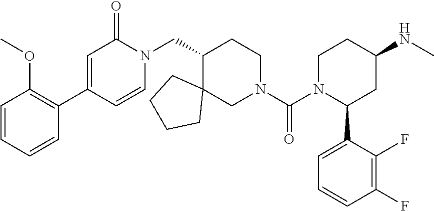 [SMILES: CN[C@@H]1CCN(C(=O)N2CC[C@@H](Cn3ccc(-c4ccccc4OC)cc3=O)C3(CCCC3)C2)[C@H](c2cccc(F)c2F)C1] | 1-(((R)-7-((2S,4R)-2-(2,3- Difluorophenyl)-4- (methylamino)piperidine- 1-carbonyl)-7- azaspiro[4.5]decan-10- yl)methyl)-4-(2- methoxyphenyl)pyridin- 2(1H)-one | 7.62 (d, J = 7.0 Hz, 1H), 7.41 (ddd, J = 8.7, 7.4, 1.7 Hz, 1H), 7.34 (dd, J = 7.6, 1.7 Hz, 1H), 7.20 (tq, J = 7.8, 5.1, 3.9 Hz, 1H), 7.13 (dd, J = 8.5, 1.0 Hz, 1H), 7.11- 7.00 (m, 3H), 6.46 (d, J = 1.9 Hz, 1H), 6.36 (dd, J = 7.0, 2.0 Hz, 1H), 4.29 (dd, J = 12.1, 2.9 Hz, 1H), 4.06 (dd, J = 12.6, 3.0 Hz, 1H), 3.84 (d, J = 13.6 Hz, 1H), 3.80 (s, 3H), 3.70 (s, 1H), 3.35 (d, J = 12.3 Hz, 1H), 3.29 (d, J = 6.5 Hz, 1H), 2.90 (s, 1H), 2.69 (dd, J = 21.2, 11.7 Hz, 2H), 2.44 (d, J = 10.4 Hz, 1H), 2.25 (s, 3H), 1.93 (d, J = 12.5 Hz, 2H), 1.84 (td, J = | (Method B) R_{T} = 0.96 min, m/z 605 [M + H]⁺ |
| | | | 10.3, 8.9, 5.1 Hz, 1H), 1.76 (s, 1H), | |
| | | | 1.71-1.42 (m, 5H), 1.40-1.28 (m, 3H), | |
| | | | 1.28-1.11 (m, 4H). | |
| 132 | 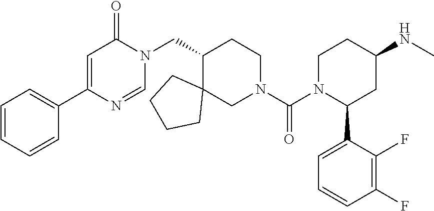 [SMILES: CN[C@@H]1CCN(C(=O)N2CC[C@@H](Cn3cnc(-c4ccccc4)cc3=O)C3(CCCC3)C2)[C@H](c2cccc(F)c2F)C1] | 3-(((R)-7-((2S,4R)-2-(2,3- Difluorophenyl)-4- (methylamino)piperidine- 1-carbonyl)-7- azaspiro[4.5]decan-10- yl)methyl)-6- phenylpyrimidin-4(3H)- one | 8.55 (s, 1H), 8.06 (dd, J = 6.7, 3.0 Hz, 2H), 7.49 (dd, J = 5.1, 2.0 Hz, 3H), 7.20 (td, J = 9.0, 8.4, 5.6 Hz, 1H), 7.06 (td, J = 11.2, 9.7, 5.8 Hz, 2H), 6.97 (s, 1H), 4.29 (dd, J = 12.0, 2.9 Hz, 1H), 4.10 (dd, J = 13.1, 3.1 Hz, 1H), 3.90-3.79 (m, 1H), 3.71 (t, J = 12.1 Hz, 1H), 3.39 (d, J = 13.0 Hz, 1H), 3.34-3.26 (m, 2H, obscured by HDO signal), 2.85 (s, 1H), 2.68 (dd, J = 12.9, 9.1 Hz, 2H), 2.47-2.42 (m, 1H), 2.26 (s, 3H), 1.94 (d, J = 12.5 Hz, 2H), 1.86 (ddt, J = 11.6, 8.0, 3.9 Hz, 1H), 1.76 (s, 1H), 1.72-1.42 (m, 4H), 1.35 (ddd, J = | (Method B) R_{T} = 0.95 min, m/z 576 [M + H]⁺ |
| | | | 18.1, 10.3, 6.6 Hz, 3H), 1.21 (ddd, J = | |
| | | | 22.7, 15.9, 8.6 Hz, 4H). | |
| 133 | 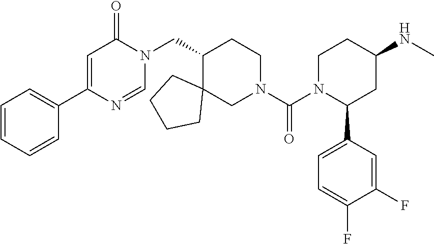 [SMILES: CN[C@@H]1CCN(C(=O)N2CC[C@@H](Cn3cnc(-c4ccccc4)cc3=O)C3(CCCC3)C2)[C@H](c2ccc(F)c(F)c2)C1] | 3-(((R)-7-((2S,4R)-2-(3,4- Difluorophenyl)-4- (methylamino)piperidine- 1-carbonyl)-7- azaspiro[4.5]decan-10- yl)methyl)-6- phenylpyrimidin-4(3H)- one | 8.54 (s, 1H), 8.09-8.02 (m, 2H), 7.49 (dd, J = 5.1, 1.9 Hz, 3H), 7.28 (dt, J = 10.7, 8.5 Hz, 1H), 7.19 (ddd, J = 12.0, 7.9, 2.1 Hz, 1H), 7.04 (dd, J = 9.6, 3.9 Hz, 1H), 6.97 (s, 1H), 4.10 (dd, J = 13.1, 3.1 Hz, 1H), 3.98 (dd, J = 11.7, 3.0 Hz, 1H), 3.85 (dt, J = 13.2, 4.5 Hz, 1H), 3.71 (t, J = 12.0 Hz, 1H), 3.41 (d, J = 12.9 Hz, 1H), 3.25 (dt, J = 12.2, 3.7 Hz, 1H), 2.84 (s, 1H), 2.74-2.64 (m, 2H, obscured by DMSO satellite), 2.57-2.52 (m, 1H, obscured by DMSO signal), 2.43 (td, J = 10.6, 5.3 Hz, 1H), 2.24 (s, 3H), 1.96- 1.81 (m, 3H), 1.77 (s, 1H), 1.71-1.59 (m, 2H), 1.59-1.44 (m, 2H), 1.34 (td, J = | (Method B) R_{T} = 0.95 min, m/z 576 [M + H]⁺ |
| | | | 15.0, 13.8, 7.4 Hz, 3H), 1.20 (qd, J = | |
| | | | 12.5, 12.0, 7.5 Hz, 4H). | |
| 134 | 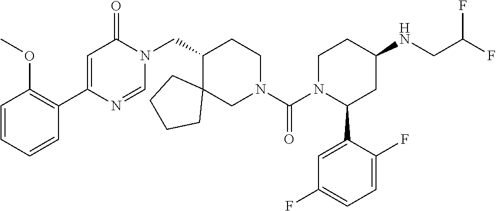 [SMILES: COc1ccccc1-c1cc(=O)n(C[C@@H]2CCN(C(=O)N3CC[C@@H](NCC(F)F)C[C@H]3c3cc(F)ccc3F)CC23CCCC3)cn1] | 3-(((R)-7-((2S,4R)-4- ((2,2- Difluoroethyl)amino)-2- (2,5- difluorophenyl)piperidine- 1-carbonyl)-7- azaspiro[4.5]decan-10- yl)methyl)-6-(2- methoxyphenyl)pyrimidin- 4(3H)-one | 8.50 (s, 1H), 7.95 (dd, J = 7.8, 1.9 Hz, 1H), 7.45 (ddd, J = 8.7, 7.3, 1.9 Hz, 1H), 7.19-7.10 (m, 2H), 7.09-6.93 (m, 4H), 5.91 (tt, J = 56.7, 4.4 Hz, 1H), 4.24 (d, J = 12.1 Hz, 1H), 4.09 (dd, J = 13.1, 3.0 Hz, 1H), 3.92-3.81 (m, 4H), 3.72 (t, J = 13.2 Hz, 1H), 3.40 (d, J = 12.3 Hz, 1H), 3.28- 3.23 (m, 1H, obscured by HDO signal), 2.98-2.79 (m, 3H), 2.75 (d, J = 12.9 Hz, 1H), 2.69-2.57 (m, 2H, obscured by DMSO satellite), 2.09-1.12 (m, 16H). | (Method B) R_{T} = 0.98 min, m/z 656 [M + H]⁺ |
| 135 | 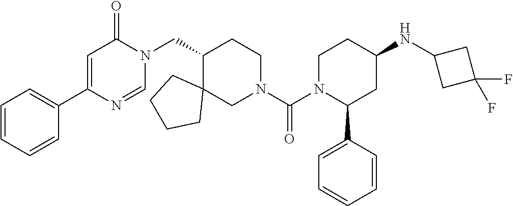 [SMILES: O=C(N1CC[C@@H](Cn2cnc(-c3ccccc3)cc2=O)C2(CCCC2)C1)N1CC[C@@H](NC2CC(F)(F)C2)C[C@H]1c1ccccc1] | 3-(((R)-7-((2S,4R)-4- ((3,3- Difluorocyclobutyl)amino)- 2-phenylpiperidine-1- carbonyl)-7- azaspiro[4.5]decan-10- yl)methyl)-6- phenylpyrimidin-4(3H)- one | 8.55 (s, 1H), 8.10-8.03 (m, 2H), 7.49 (dd, J = 5.1, 1.9 Hz, 3H), 7.25-7.16 (m, 4H), 7.16-7.11 (m, 1H), 6.97 (d, J = 0.8 Hz, 1H), 4.10 (dd, J = 13.0, 3.1 Hz, 1H), 3.93 (dd, J = 11.8, 3.1 Hz, 1H), 3.87 (dd, J = 12.7, 3.3 Hz, 1H), 3.71 (t, J = 12.0 Hz, 1H), 3.43 (d, J = 13.0 Hz, 1H), 3.27- 3.11 (m, 2H), 2.84 (s, 1H), 2.79-2.69 (m, 2H), 2.69-2.60 (m, 2H, obscured by DMSO satellite), 2.60-2.54 (m, 1H, obscured by DMSO signal), 2.26 (s, 3H), 1.93-1.80 (m, 3H), 1.76 (s, 1H), 1.70- 1.43 (m, 4H), 1.43-1.11 (m, 7H). | (Method B) R_{T} = 0.98 min, m/z 616 [M + H]⁺ |
| 136 | 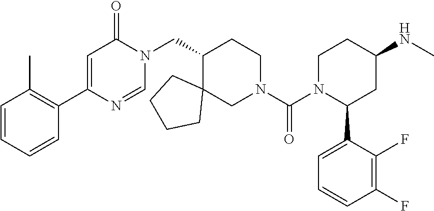 [SMILES: CN[C@@H]1CCN(C(=O)N2CC[C@@H](Cn3cnc(-c4ccccc4C)cc3=O)C3(CCCC3)C2)[C@H](c2cccc(F)c2F)C1] | 3-(((R)-7-((2S,4R)-2-(2,3- Difluorophenyl)-4- (methylamino)piperidine- 1-carbonyl)-7- azaspiro[4.5]decan-10- yl)methyl)-6-(0- tolyl)pyrimidin-4(3H)-one | 8.52 (s, 1H), 7.41 (dd, 1H), 7.34 (td, 1H), 7.31-7.24 (m, 2H), 7.23-7.15 (m, 1H), 7.11-7.02 (m, 2H), 6.46 (s, 1H), 4.30 (dd, 1H), 4.11 (dd, 1H), 3.85 (dt, 1H), 3.73 (t, 1H), 3.42-3.32 (m, 2H), 3.02-2.77 (m, 1H), 2.74-2.65 (m, 2H), 2.46-2.41 (m, 1H), 2.36 (s, 3H), 2.25 (s, 3H), 1.93 (d, 2H), 1.90-1.82 (m, 1H), 1.81-1.70 (m, 1H), 1.70-1.42 (m, 5H), 1.42-1.29 (m, 3H), 1.29-1.12 (m, 4H). | (Method B) R_{T} = 0.95 min, m/z 590 [M + H]⁺ |
| 137 | 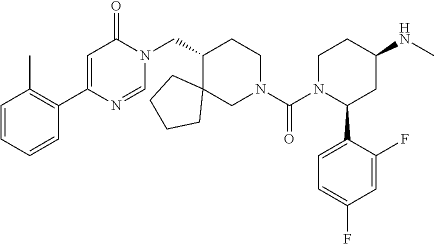 [SMILES: CN[C@@H]1CCN(C(=O)N2CC[C@@H](Cn3cnc(-c4ccccc4C)cc3=O)C3(CCCC3)C2)[C@H](c2ccc(F)cc2F)C1] | 3-(((R)-7-((2S,4R)-2-(2,4- Difluorophenyl)-4- (methylamino)piperidine- 1-carbonyl)-7- azaspiro[4.5]decan-10- yl)methyl)-6-(o- tolyl)pyrimidin-4(3H)-one | 8.52 (s, 1H), 7.41 (d, 1H), 7.34 (t, 1H), 7.31-7.21 (m, 3H), 7.09 (t, 1H), 6.95 (t, 1H), 6.46 (s, 1H), 4.24 (d, 1H), 4.11 (dd, 1H), 3.85 (d, 1H), 3.73 (t, 1H), 3.44-3.36 (m, 1H), 3.27-3.20 (m, 1H), 2.99-2.78 (m, 1H), 2.75-2.65 (m, 2H), 2.44-2.39 (m, 1H), 2.36 (s, 3H), 2.24 (s, 3H), 1.99-1.81 (m, 3H), 1.81-1.70 (m, 1H), 1.70-1.58 (m, 3H), 1.57-1.42 (m, 2H), 1.42-1.28 (m, 3H), 1.28-1.10 (m, 4H). | (Method B) R_{T} = 0.94 min, m/z 590 [M + H]⁺ |
| 138 | 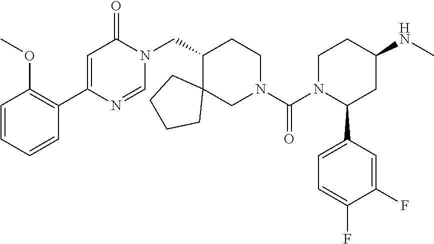 [SMILES: CN[C@@H]1CCN(C(=O)N2CC[C@@H](Cn3cnc(-c4ccccc4OC)cc3=O)C3(CCCC3)C2)[C@H](c2ccc(F)c(F)c2)C1] | 3-(((R)-7-((2S,4R)-2-(3,4- Difluorophenyl)-4- (methylamino)piperidine- 1-carbonyl)-7- azaspiro[4.5]decan-10- yl)methyl)-6-(2- methoxyphenyl)pyrimidin- 4(3H)-one | 8.49 (s, 1H), 7.95 (dd, 1H), 7.45 (td, 1H), 7.27 (dt, 1H), 7.22-7.14 (m, 2H), 7.09- 7.01 (m, 2H), 6.99 (s, 1H), 4.08 (dd, 1H), 3.98 (dd, 1H), 3.11 (s, 3H), 3.85 (dt, 1H), 3.70 (t, 1H), 3.40 (d, 1H), 3.25 (dt, 1H), 2.94-2.76 (br s, 1H), 2.74-2.63 (m, 2H), 2.43 (tt, 1H), 2.24 (s, 3H), 1.96-1.81 (m, 3H), 1.81-1.43 (m, 6H), 1.41-1.27 (m, 3H), 1.27-1.12 (m, 4H). | (Method B) R_{T} = 0.95 min, m/z 606 [M + H]⁺ |
| 139 | 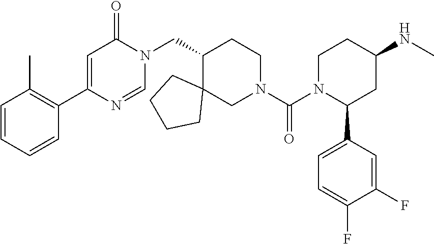 [SMILES: CN[C@@H]1CCN(C(=O)N2CC[C@@H](Cn3cnc(-c4ccccc4C)cc3=O)C3(CCCC3)C2)[C@H](c2ccc(F)c(F)c2)C1] | 3-(((R)-7-((2S,4R)-2-(3,4- Difluorophenyl)-4- (methylamino)piperidine- 1-carbonyl)-7- azaspiro[4.5]decan-10- yl)methyl)-6-(0- tolyl)pyrimidin-4(3H)-one | 8.51 (s, 1H), 7.41 (d, 1H), 7.37-7.31 (m, 1H), 7.31-7.24 (m, 3H), 7.19 (dd, 1H), 7.08-7.01 (m, 1H), 6.46 (s, 1H), 4.10 (dd, 1H), 3.98 (dd, 1H), 3.91-3.81 (m, 1H), 3.73 (t, 1H), 3.42-3.36 (m, 1H), 3.27-3.23 (m, 1H), 2.99-2.78 (m, 1H), 2.72 (d, 1H), 2.65 (t, 1H), 2.45-2.39 (m, 1H), 2.36 (s, 3H), 2.24 (s, 3H), 1.95-1.83 (m, 3H), 1.81- 1.71 (m, 1H), 1.70-1.44 (m, 5H), 1.40- 1.28 (m, 3H), 1.28-1.12 (m, 4H). | (Method B) R_{T} = 0.97 min, m/z 590 [M + H]⁺ |
| 140 | 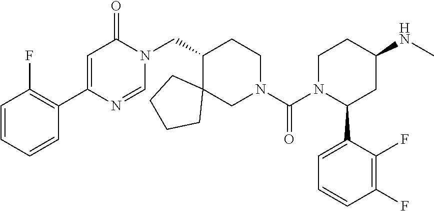 [SMILES: CN[C@@H]1CCN(C(=O)N2CC[C@@H](Cn3cnc(-c4ccccc4F)cc3=O)C3(CCCC3)C2)[C@H](c2cccc(F)c2F)C1] | 3-(((R)-7-((2S,4R)-2-(2,3- Difluorophenyl)-4- (methylamino)piperidine- 1-carbonyl)-7- azaspiro[4.5]decan-10- yl)methyl)-6-(2- fluorophenyl)pyrimidin-4(3H)- one | 8.58 (s, 1H), 8.01 (td, J = 7.9, 1.8 Hz, 1H), 7.55 (dddd, J = 8.2, 7.2, 5.0, 1.8 Hz, 1H), 7.39-7.31 (m, 2H), 7.23-7.15 (m, 1H), 7.11-7.02 (m, 2H), 6.79 (d, J = 1.0 Hz, 1H), 4.29 (dd, J = 12.0, 2.9 Hz, 1H), 4.10 (dd, J = 13.0, 3.0 Hz, 1H), 3.85 (dt, J = 12.6, 3.8 Hz, 1H), 3.72 (t, J = 12.0 Hz, 1H), 3.39 (d, J = 13.0 Hz, 1H), 3.29- 3.25 (m, 1H), 2.86 (s, 1H), 2.68 (t, J = 11.2 Hz, 2H), 2.47-2.39 (m, 1H), 2.25 (s, 3H), 1.93 (d, J = 12.8 Hz, 2H), 1.85 (ddt, J = 10.5, 6.8, 3.4 Hz, 1H), 1.76 (s, 1H), 1.71-1.42 (m, 5H), 1.35 (dtd, J = | (Method B) R_{T} = 0.94 min, m/z 594 [M + H]⁺ |
| | | | 16.2, 9.8, 7.1, 4.1 Hz, 3H), 1.28-1.13 | |
| | | | (m, 4H). | |
| 141 | 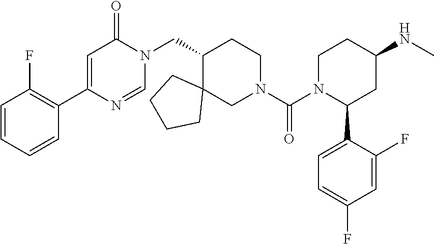 [SMILES: CN[C@@H]1CCN(C(=O)N2CC[C@@H](Cn3cnc(-c4ccccc4F)cc3=O)C3(CCCC3)C2)[C@H](c2ccc(F)cc2F)C1] | 3-(((R)-7-((2S,4R)-2-(2,4- Difluorophenyl)-4- (methylamino)piperidine- 1-carbonyl)-7- azaspiro[4.5]decan-10- yl)methyl)-6-(2- fluorophenyl)pyrimidin-4(3H)- one | 8.58 (s, 1H), 8.01 (td, J = 7.9, 1.8 Hz, 1H), 7.58-7.51 (m, 1H), 7.39-7.31 (m, 2H), 7.26 (td, J = 8.5, 6.7 Hz, 1H), 7.08 (ddd, J = 10.7, 9.4, 2.6 Hz, 1H), 6.95 (td, J = 8.5, 2.6 Hz, 1H), 6.79 (d, J = 1.0 Hz, 1H), 4.24 (dd, J = 12.0, 3.0 Hz, 1H), 4.10 (dd, J = 13.0, 3.0 Hz, 1H), 3.90-3.80 (m, 1H), 3.72 (t, J = 12.0 Hz, 1H), 3.38 (d, J = 12.9 Hz, 1H), 3.29-3.23 (m, 1H), 2.86 (s, 1H), 2.72-2.65 (m, 2H), 2.47-2.39 (m, 1H), 2.24 (s, 3H), 1.96-1.82 (m, 3H), 1.75 (d, J = 8.4 Hz, 1H), 1.71-1.43 (m, 5H), 1.35 (dq, J = 18.9, 8.0, 6.0 Hz, 3H), 1.19 (dq, J = 20.5, 7.5 Hz, 4H). | (Method B) R_{T} = 0.94 min, m/z 594 [M + H]⁺ |
| 142 | 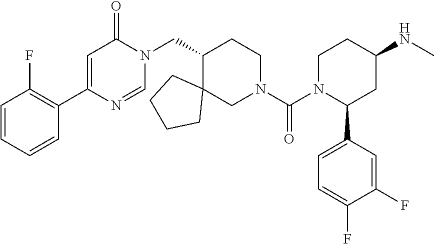 [SMILES: CN[C@@H]1CCN(C(=O)N2CC[C@@H](Cn3cnc(-c4ccccc4F)cc3=O)C3(CCCC3)C2)[C@H](c2ccc(F)c(F)c2)C1] | 3-(((R)-7-((2S,4R)-2-(3,4- Difluorophenyl)-4- (methylamino)piperidine- 1-carbonyl)-7- azaspiro[4.5]decan-10- yl)methyl)-6-(2- fluorophenyl)pyrimidin-4(3H)- one | 8.57 (s, 1H), 8.01 (td, J = 7.9, 1.8 Hz, 1H), 7.54 (tdd, J = 7.6, 5.0, 1.8 Hz, 1H), 7.38-7.31 (m, 2H), 7.28 (dt, J = 10.6, 8.4 Hz, 1H), 7.19 (ddd, J = 12.0, 7.8, 2.1 Hz, 1H), 7.08-7.00 (m, 1H), 6.79 (s, 1H), 4.10 (dd, J = 13.1, 3.0 Hz, 1H), 3.98 (dd, J = 11.9, 3.0 Hz, 1H), 3.85 (dt, J = 13.2, 4.4 Hz, 1H), 3.72 (t, J = 12.0 Hz, 1H), 3.41 (d, J = 12.9 Hz, 1H), 3.28-3.20 (m, 1H), 2.85 (s, 1H), 2.74-2.65 (m, 2H), 2.43 (dq, J = 10.7, 6.6, 5.3 Hz, 1H), 2.24 (s, 3H), 1.95-1.81 (m, 3H), 1.77 (s, 1H), 1.71-1.42 (m, 5H), 1.41-1.27 (m, 3H), 1.20 (tt, J = 12.3, 8.3 Hz, 4H). | (Method B) R_{T} = 0.95 min, m/z 594 [M + H]⁺ |
| 143 | 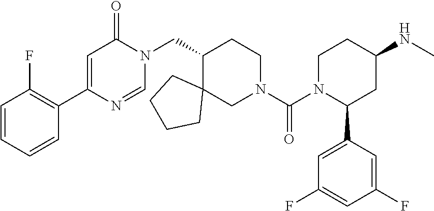 [SMILES: CN[C@@H]1CCN(C(=O)N2CC[C@@H](Cn3cnc(-c4ccccc4F)cc3=O)C3(CCCC3)C2)[C@H](c2cc(F)cc(F)c2)C1] | 3-(((R)-7-((2S,4R)-2-(3,5- Difluorophenyl)-4- (methylamino)piperidine- 1-carbonyl)-7- azaspiro[4.5]decan-10- yl)methyl)-6-(2- fluorophenyl)pyrimidin-4(3H)- one | 8.58 (s, 1H), 8.01 (td, J = 7.9, 1.9 Hz, 1H), 7.54 (tdd, J = 7.6, 5.0, 1.8 Hz, 1H), 7.38-7.32 (m, 2H), 6.98 (tt, J = 9.3, 2.4 Hz, 1H), 6.92-6.86 (m, 2H), 6.79 (s, 1H), 4.11 (dd, J = 13.1, 3.0 Hz, 1H), 4.01 (dd, J = 11.7, 3.1 Hz, 1H), 3.86 (dt, J = 12.7, 3.6 Hz, 1H), 3.78-3.67 (m, 1H), 3.41 (d, J = 12.9 Hz, 1H), 3.27 (dt, J = 12.0, 4.5 Hz, 1H), 2.86 (s, 1H), 2.74 (d, J = 12.9 Hz, 1H), 2.69-2.60 (m, 1H, obscured by DMSO satellite), 2.43 (dq, J = 10.5, 6.4, 5.1 Hz, 1H), 2.24 (s, 3H), 1.98-1.83 (m, 3H), 1.78 (s, 1H), 1.71-1.43 (m, 5H), 1.41-1.11 (m, 7H). | (Method B) R_{T} = 0.95 min, m/z 594 [M + H]⁺ |
| 144 | 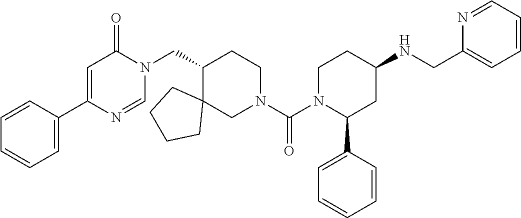 [SMILES: O=C(N1CC[C@@H](Cn2cnc(-c3ccccc3)cc2=O)C2(CCCC2)C1)N1CC[C@@H](NCc2ccccn2)C[C@H]1c1ccccc1] | 6-Phenyl-3-(((R)-7- ((2S,4R)-2-phenyl-4- ((pyridin-2- ylmethyl)amino)piperidine- 1-carbonyl)-7- azaspiro[4.5]decan-10- yl)methyl)pyrimidin-4(3H)- one | 8.55 (s, 1H), 8.46 (ddd, J = 4.9, 1.9, 0.9 Hz, 1H), 8.09-8.03 (m, 2H), 7.72 (td, J = 7.6, 1.8 Hz, 1H), 7.49 (dd, J = 5.1, 1.9 Hz, 3H), 7.40 (dd, J = 7.8, 1.1 Hz, 1H), 7.24- 7.18 (m, 5H), 7.16-7.11 (m, 1H), 6.97 (s, 1H), 4.10 (dd, J = 13.1, 3.0 Hz, 1H), 3.95 (dd, J = 11.7, 3.0 Hz, 1H), 3.87 (dd, J = 11.1, 6.7 Hz, 1H), 3.80 (d, J = 5.5 Hz, 2H), 3.70 (t, J = 12.0 Hz, 1H), 3.44 (d, J = 12.9 Hz, 1H), 3.26 (dt, J = 11.8, 3.6 Hz, 1H), 2.83 (s, 1H), 2.70-2.58 (m, 3H, obscured by DMSO satellite), 2.23 (q, J = 6.6 Hz, 1H), 2.00-1.92 (m, 2H), 1.84 | (Method B) R_{T} = 0.96 min, m/z 617 [M + H]⁺ |
| | | | (ddt, J = 10.7, 7.0, 3.4 Hz, 1H), 1.77 (s, | |
| | | | 1H), 1.71-1.38 (m, 5H), 1.38-1.14 (m, | |
| | | | 6H) | |
| 145 | 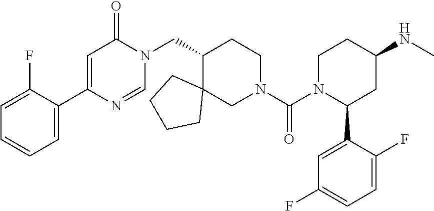 [SMILES: CN[C@@H]1CCN(C(=O)N2CC[C@@H](Cn3cnc(-c4ccccc4F)cc3=O)C3(CCCC3)C2)[C@H](c2cc(F)ccc2F)C1] | 3-(((R)-7-((2S,4R)-2-(2,5- Difluorophenyl)-4- (methylamino)piperidine- 1-carbonyl)-7- azaspiro[4.5]decan-10- yl)methyl)-6-(2- fluorophenyl)pyrimidin-4(3H)- one | 8.58 (s, 1H), 8.01 (td, J = 7.9, 1.8 Hz, 1H), 7.54 (tdd, J = 7.5, 5.0, 1.8 Hz, 1H), 7.39-7.31 (m, 2H), 7.14 (td, J = 9.3, 4.5 Hz, 1H), 7.06-6.95 (m, 2H), 6.79 (s, 1H), 4.29-4.22 (m, 1H), 4.11 (dd, J = 13.0, 3.0 Hz, 1H), 3.86 (dt, J = 13.4, 4.3 Hz, 1H), 3.73 (t, J = 12.0 Hz, 1H), 3.40 (d, J = 12.9 Hz, 1H), 3.27 (d, J = 3.3 Hz, 1H), 2.87 (s, 1H), 2.75 (d, J = 12.9 Hz, 1H), 2.71-2.61 (m, 1H), 2.43 (td, J = 10.7, 5.2 Hz, 1H), 2.25 (s, 3H), 1.96-1.83 (m, 3H), 1.77 (s, 1H), 1.71-1.43 (m, 5H), 1.41-1.13 (m, 7H). | (Method B) R_{T} = 0.93 min, m/z 594 [M + H]⁺ |
| 146 | 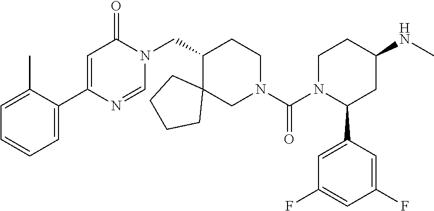 [SMILES: CN[C@@H]1CCN(C(=O)N2CC[C@@H](Cn3cnc(-c4ccccc4C)cc3=O)C3(CCCC3)C2)[C@H](c2cc(F)cc(F)c2)C1] | 3-(((R)-7-((2S,4R)-2-(3,5- Difluorophenyl)-4- (methylamino)piperidine- 1-carbonyl)-7- azaspiro[4.5]decan-10- yl)methyl)-6-(o- tolyl)pyrimidin-4(3H)-one | 8.52 (s, 1H), 7.41 (d, 1H), 7.34 (t, 1H), 7.32-7.24 (m, 2H), 6.99 (t, 1H), 6.90 (d, 2H), 6.46 (s, 1H), 4.11 (d, 1H), 4.02 (dd, 1H), 3.92-3.82 (m, 1H), 3.74 (t, 1H), 3.43- 3.36 (m, 1H), 3.28-3.24 (m, 1H), 3.01- 2.82 (m, 1H), 2.81-2.71 (d, 1H), 2.66 (t, 1H), 2.36 (s, 3H), 2.28 (s, 3H), 2.00-1.84 (m, 3H), 1.82-1.71 (m, 1H), 1.71-160 (m, 2H), 1.60-1.44 (m, 2H), 1.44-1.12 (m, 8H). [NH proton not observed]. | (Method B) R_{T} = 0.95 min, m/z 590 [M + H]⁺ |
| 147 | 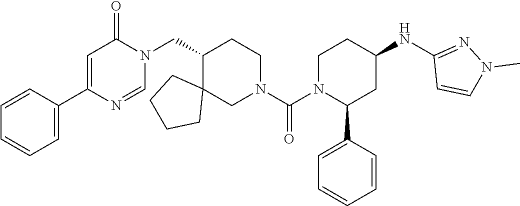 [SMILES: Cn1ccc(N[C@@H]2CCN(C(=O)N3CC[C@@H](Cn4cnc(-c5ccccc5)cc4=O)C4(CCCC4)C3)[C@H](c3ccccc3)C2)n1] | 3-(((R)-7-((2S,4R)-4-((1- Methyl-1H-pyrazol-3- yl)amino)-2- phenylpiperidine-1- carbonyl)-7- azaspiro[4.5]decan-10- yl)methyl)-6- phenylpyrimidin-4(3H)-one | 8.55 (s, 1H), 8.10-8.02 (m, 2H), 7.49 (dd, J = 5.1, 1.9 Hz, 3H), 7.26 (d, J = 2.2 Hz, 1H), 7.24-7.16 (m, 4H), 7.16-7.11 (m, 1H), 6.97 (s, 1H), 5.38 (d, J = 2.2 Hz, 1H), 4.91 (d, J = 8.2 Hz, 1H), 4.10 (dd, J = 13.1, 3.1 Hz, 1H), 3.99 (dd, J = 11.8, 3.1 Hz, 1H), 3.88 (d, J = 13.4 Hz, 1H), 3.71 (t, J = 11.9 Hz, 1H), 3.57 (s, 3H), 3.45 (d, J = 12.7 Hz, 1H), 3.35-3.22 (m, 2H), 2.85 (s, 1H), 2.75-2.65 (m, 2H), 2.10-1.97 (m, 2H), 1.85 (ddt, J = 10.6, 7.0, 3.4 Hz, 1H), 1.77 (s, 1H), 1.72-1.43 (m, 5H), 1.32 (td, J = 13.4, 11.8, 6.6 Hz, 4H), 1.20 (dq, J = 13.9, 8.1, 6.9 Hz, 2H). | (Method B) R_{T} = 1.28 min, m/z 606 [M + H]⁺ |
| 148 | 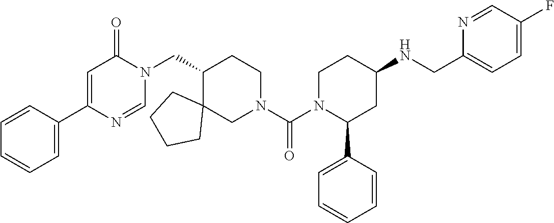 [SMILES: O=C(N1CC[C@@H](Cn2cnc(-c3ccccc3)cc2=O)C2(CCCC2)C1)N1CC[C@@H](NCc2ccc(F)cn2)C[C@H]1c1ccccc1] | 3-(((R)-7-((2S,4R)-4-(((5- Fluoropyridin-2- yl)methyl)amino)-2- phenylpiperidine-1- carbonyl)-7- azaspiro[4.5]decan-10- yl)methyl)-6- phenylpyrimidin-4(3H)-one | 8.55 (s, 1H), 8.44 (d, J = 3.0 Hz, 1H), 8.06 (dd, J = 6.7, 3.0 Hz, 2H), 7.65 (td, J = 8.8, 3.0 Hz, 1H), 7.48 (ddd, J = 10.8, 6.1, 3.5 Hz, 4H), 7.25-7.17 (m, 4H), 7.17-7.10 (m, 1H), 6.97 (s, 1H), 4.10 (dd, J = 13.0, 3.0 Hz, 1H), 3.94 (dd, J = 11.7, 3.0 Hz, 1H), 3.91-3.83 (m, 1H), 3.80 (d, J = 5.6 Hz, 2H), 3.69 (d, J = 12.1 Hz, 1H), 3.44 (d, J = 12.8 Hz, 1H), 3.25 (dd, J = 12.3, 3.8 Hz, 1H), 2.83 (s, 1H), 2.69-2.55 (m, 3H), 2.25 (d, J = 6.6 Hz, 1H), 1.99-1.90 (m, 2H), 1.84 (ddt, J = 10.7, 7.1, 3.7 Hz, 1H), 1.77 (s, 1H), 1.71- 1.37 (m, 5H), 1.31 (q, J = 10.9, 9.5 Hz, 4H), 1.18 (dt, J = 13.8, 7.1 Hz, 2H). | (Method B) R_{T} = 0.99 min, m/z 635 [M + H]⁺ |
| 149 | 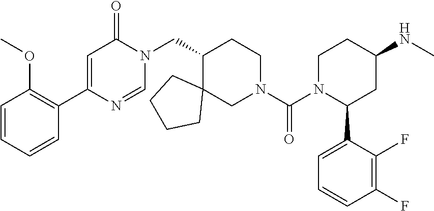 [SMILES: CN[C@@H]1CCN(C(=O)N2CC[C@@H](Cn3cnc(-c4ccccc4OC)cc3=O)C3(CCCC3)C2)[C@H](c2cccc(F)c2F)C1] | 3-(((R)-7-((2S,4R)-2-(2,3- Difluorophenyl)-4- (methylamino)piperidine- 1-carbonyl)-7- azaspiro[4.5]decan-10- yl)methyl)-6-(2- methoxyphenyl)pyrimidin- 4(3H)-one | 8.50 (s, 1H), 7.95 (dd, 1H), 7.45 (t, 1H), 7.25-7.13 (m, 2H), 7.11-7.02 (m, 3H), 6.99 (s, 1H), 4.29 (d, 1H), 4.08 (d, 1H), 3.93-3.78 (m, 4H), 3.69 (t, 1H), 3.41-3.35 (m, 1H), 2.96-2.76 (m, 1H), 2.68 (t, 2H), 2.25 (s, 3H), 1.93 (d, 2H), 1.88-1.80 (m, 1H), 1.80-1.71 (m, 1H), 1.71-1.42 (m, 5H), 1.40-1.28 (m, 3H), 1.28-1.13 (m, 4H). [Apparent signal at 3.27 ppm cannot be resolved or integrated but likely to account for 1H; NH proton not observed] | (Method B) R_{T} = 0.94 min, m/z 606 [M + H]⁺ |
| 150 | 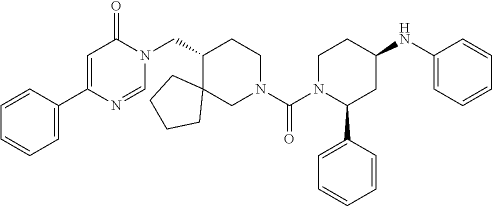 [SMILES: O=C(N1CC[C@@H](Cn2cnc(-c3ccccc3)cc2=O)C2(CCCC2)C1)N1CC[C@@H](Nc2ccccc2)C[C@H]1c1ccccc1] | 6-Phenyl-3-(((R)-7- ((2S,4R)-2-phenyl-4- (phenylamino)piperidine- 1-carbonyl)-7- azaspiro[4.5]decan-10- yl)methyl)pyrimidin- 4(3H)-one | 8.55 (s, 1H), 8.09-8.03 (m, 2H), 7.49 (dd, J = 5.1, 1.9 Hz, 3H), 7.22 (d, J = 6.1 Hz, 4H), 7.14 (td, J = 6.0, 2.6 Hz, 1H), 7.04 (dd, J = 8.5, 7.2 Hz, 2H), 6.97 (d, J = 0.8 Hz, 1H), 6.60-6.55 (m, 2H), 6.52- 6.47 (m, 1H), 5.37 (d, J = 8.3 Hz, 1H), 4.09 (ddd, J = 14.7, 12.6, 3.1 Hz, 2H), 3.89 (d, J = 13.3 Hz, 1H), 3.71 (t, J = 12.0 Hz, 1H), 3.44 (q, J = 11.2, 8.5 Hz, 2H), 3.29 (s, 1H), 2.94-2.75 (m, 2H), 2.69 (d, J = 12.9 Hz, 1H), 2.10-1.96 (m, 2H), 1.90-1.82 (m, 1H), 1.78 (s, 1H), 1.73- 1.45 (m, 5H), 1.41-1.26 (m, 4H), 1.20 (td, J = 13.9, 13.4, 6.6 Hz, 2H). | (Method B) R_{T} = 1.59 min, m/z 602 [M + H]⁺ |
| 151 | 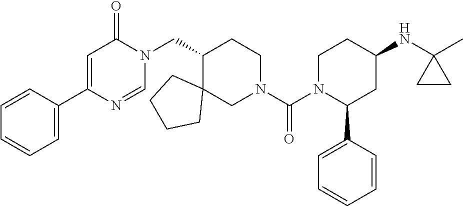 [SMILES: CC1(N[C@@H]2CCN(C(=O)N3CC[C@@H](Cn4cnc(-c5ccccc5)cc4=O)C4(CCCC4)C3)[C@H](c3ccccc3)C2)CC1] | 3-(((R)-7-((2S,4R)-4-((1- Methylcyclopropyl)amino)- 2-phenylpiperidine-1- carbonyl)-7- azaspiro[4.5]decan-10- yl)methyl)-6- phenylpyrimidin-4(3H)- one | 8.54 (s, 1H), 8.06 (dd, J = 6.8, 3.0 Hz, 2H), 7.49 (dd, J = 5.0, 1.9 Hz, 3H), 7.24- 7.16 (m, 4H), 7.16-7.10 (m, 1H), 6.97 (s, 1H), 4.10 (dd, J = 13.1, 3.0 Hz, 1H), 3.95 (dd, J = 11.9, 3.1 Hz, 1H), 3.88 (dt, J = 13.4, 4.4 Hz, 1H), 3.70 (t, J = 12.0 Hz, 1H), 3.45 (d, J = 12.8 Hz, 1H), 3.22 (dt, J = 12.1, 3.8 Hz, 1H), 2.93-2.74 (m, 2H), 2.71-2.62 (m, 2H), 2.00 (s, 1H), 1.93 (d, J = 12.2 Hz, 1H), 1.90-1.81 (m, 2H), 1.76 (s, 1H), 1.72-1.42 (m, 4H), 1.32 (dtt, J = 25.3, 14.3, 6.4 Hz, 4H), 1.24- | (Method B) R_{T} = 0.96 min, m/z 580 [M + H]⁺ |
| | | | 1.11 (m, 6H), 0.41 (ddd, J = 8.4, 5.3, | |
| | | | 2.8 Hz, 1H), 0.35-0.20 (m, 3H). | |
| 152 | 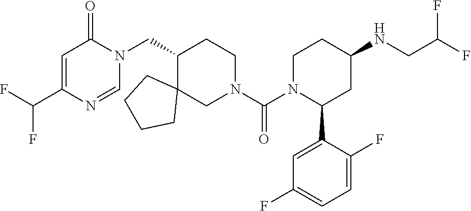 [SMILES: O=C(N1CC[C@@H](Cn2cnc(C(F)F)cc2=O)C2(CCCC2)C1)N1CC[C@@H](NCC(F)F)C[C@H]1c1cc(F)ccc1F] | 3-(((R)-7-((2S,4R)-4- ((2,2- Difluoroethyl)amino)-2- (2,5- difluorophenyl)piperidine- 1-carbonyl)-7- azaspiro[4.5]decan-10- yl)methyl)-6- (difluoromethyl)pyrimidin- 4(3H)-one | 8.58 (s, 1H), 7.14 (td, J = 9.3, 4.5 Hz, 1H), 7.00 (dddd, J = 23.4, 9.0, 6.4, 3.4 Hz, 2H), 6.75 (t, J = 54.2 Hz, 1H), 6.66 (s, 1H), 5.91 (tt, J = 56.5, 4.3 Hz, 1H), 4.24 (dt, J = 11.9, 2.4 Hz, 1H), 4.09 (dd, J = 13.1, 3.0 Hz, 1H), 3.84 (d, J = 13.3 Hz, 1H), 3.72 (t, J = 12.0 Hz, 1H), 3.39 (d, J = 12.6 Hz, 1H), 3.29-3.21 (m, 1H), 2.89 (tdd, J = 15.9, 7.4, 4.3 Hz, 3H), 2.73 (d, J = 12.9 Hz, 1H), 2.68-2.58 (m, 2H), 2.03- 1.96 (m, 1H), 1.96-1.88 (m, 2H), 1.83 (td, J = 11.3, 10.5, 5.2 Hz, 1H), 1.75 (s, 1H), 1.69-1.10 (m, 11H). | (Method A) R_{T} = 0.80 min, m/z 600 [M + H]⁺ |
| 153 | 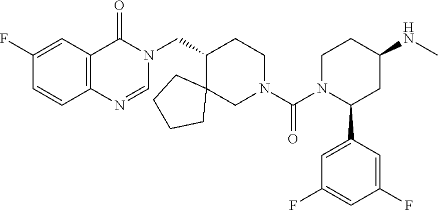 [SMILES: CN[C@@H]1CCN(C(=O)N2CC[C@@H](Cn3cnc4ccc(F)cc4c3=O)C3(CCCC3)C2)[C@H](c2cc(F)cc(F)c2)C1] | 3-(((R)-7-((2S,4R)-2-(3,5- Difluorophenyl)-4- (methylamino)piperidine- 1-carbonyl)-7- azaspiro[4.5]decan-10- yl)methyl)-6- fluoroquinazolin-4(3H)- one | 8.37 (s, 1H), 7.82 (dd, J = 8.7, 2.9 Hz, 1H), 7.80-7.69 (m, 2H), 6.98 (tt, J = 9.3, 2.4 Hz, 1H), 6.92-6.84 (m, 2H), 4.16 (dd, J = 13.4, 3.0 Hz, 1H), 4.01 (dd, J = 11.7, 3.1 Hz, 1H), 3.89-3.73 (m, 2H), 3.41 (d, J = 12.8 Hz, 1H), 3.29-3.22 (m, 1H), 2.82 (s, 1H), 2.73 (d, J = 12.9 Hz, 1H), 2.69-2.59 (m, 1H), 2.42 (td, J = 10.5, 5.2 Hz, 1H), 2.24 (s, 3H), 1.97- 1.84 (m, 3H), 1.80 (s, 1H), 1.72-1.43 (m, 5H), 1.43-1.11 (m, 7H). | (Method A) R_{T} = 0.87 min, m/z 568 [M + H]⁺ |
| 154 | 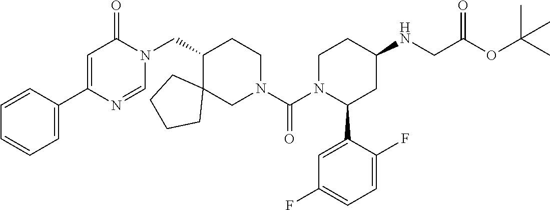 [SMILES: CC(C)(C)OC(=O)CN[C@@H]1CCN(C(=O)N2CC[C@@H](Cn3cnc(-c4ccccc4)cc3=O)C3(CCCC3)C2)[C@H](c2cc(F)ccc2F)C1] | tert-Butyl ((2S,4R)-2- (2,5-difluorophenyl)-1- ((R)-10-((6-oxo-4- phenylpyrimidin-1(6H)- yl)methyl)-7- azaspiro[4.5]decane-7- carbonyl)piperidin-4- yl)glycinate | 9.08 (s, 2H), 8.54 (s, 1H), 8.06 (dd, J = 6.6, 3.0 Hz, 2H), 7.49 (dd, J = 5.2, 2.0 Hz, 3H), 7.19 (td, J = 9.3, 4.5 Hz, 1H), 7.10 (ddd, J = 8.6, 6.5, 3.6 Hz, 1H), 7.01- 6.93 (m, 2H), 4.33-4.23 (m, 1H), 4.10 (dd, J = 12.9, 3.0 Hz, 1H), 4.01-3.80 (m, 3H), 3.73 (s, 1H), 3.46-3.34 (m, 2H), 2.85 (s, 1H), 2.77 (d, J = 12.9 Hz, 1H), 2.69 (t, J = 12.2 Hz, 1H), 2.11 (s, 2H), 1.94-1.84 (m, 1H), 1.84-1.48 (m, 7H), 1.46 (s, 9H), 1.41-1.13 (m, 5H). | (Method B) R_{T} = 1.06 min, m/z 676 [M + H]⁺ |
| 155 | 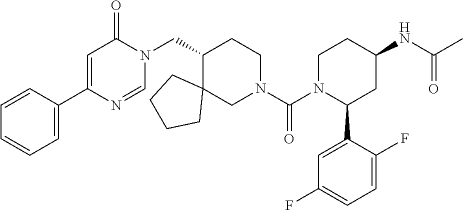 [SMILES: CC(=O)N[C@@H]1CCN(C(=O)N2CC[C@@H](Cn3cnc(-c4ccccc4)cc3=O)C3(CCCC3)C2)[C@H](c2cc(F)ccc2F)C1] | N-((2S,4R)-2-(2,5- Difluorophenyl)-1-((R)- 10-((6-oxo-4- phenylpyrimidin-1(6H)- yl)methyl)-7- azaspiro[4.5]decane-7- carbonyl)piperidin-4- yl)acetamide | 8.55 (s, 1H), 8.06 (dd, J = 6.6, 3.0 Hz, 2H), 7.80 (d, J = 7.5 Hz, 1H), 7.49 (dd, J = 5.1, 1.9 Hz, 3H), 7.15 (td, J = 9.3, 4.5 Hz, 1H), 7.04 (tt, J = 8.2, 3.4 Hz, 1H), 6.99-6.92 (m, 2H), 4.30-4.24 (m, 1H), 4.11 (dd, J = 13.1, 3.0 Hz, 1H), 3.90-3.81 (m, 1H), 3.70 (dtd, J = 11.4, 7.2, 3.5 Hz, 2H), 3.40 (d, J = 12.9 Hz, 1H), 3.28 (t, J = 3.5 Hz, 1H), 2.88 (s, 1H), 2.79-2.67 (m, 2H), 1.88 (q, J = 5.1 Hz, 3H), 1.76 (s, 4H), 1.65 (d, J = 18.9 Hz, 2H), 1.60- 1.45 (m, 3H), 1.43-1.25 (m, 4H), 1.21 (dt, J = 14.1, 6.9 Hz, 2H). | (Method B) R_{T} = 1.29 min, m/z 604 [M + H]⁺ |
| 156 | 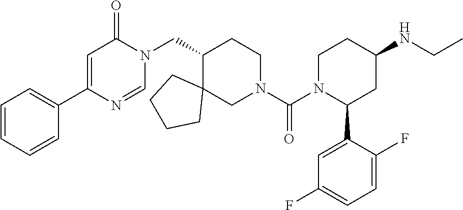 [SMILES: CCN[C@@H]1CCN(C(=O)N2CC[C@@H](Cn3cnc(-c4ccccc4)cc3=O)C3(CCCC3)C2)[C@H](c2cc(F)ccc2F)C1] | 3-(((R)-7-((2S,4R)-2-(2,5- Difluorophenyl)-4- (ethylamino)piperidine-1- carbonyl)-7- azaspiro[4.5]decan-10- yl)methyl)-6- phenylpyrimidin-4(3H)- one | 8.55 (s, 1H), 8.09-8.03 (m, 2H), 7.52-7.45 (m, 3H), 7.14 (td, 1H), 7.05-6.93 (m, 3H), 4.25 (d, 1H), 4.11 (dd, 1H), 3.85 (dt, 1H), 3.72 (t, 1H), 3.40 (d, 1H), 3.29-3.25 (m, 2H), 2.85 (br s, 1H), 2.74 (d, 1H), 2.66 (t, 1H), 2.60-2.53 (m, 2H), 1.95-1.83 (m, 3H), 1.83-1.72 (m, 1H), 1.71-1.43 (m, 5H), 1.43-1.26 (m, 4H), 1.26-1.14 (m, 3H), 0.97 (t, 3H). | (Method B) R_{T} = 0.94 min, m/z 590 [M + H]⁺ |
| 157 | 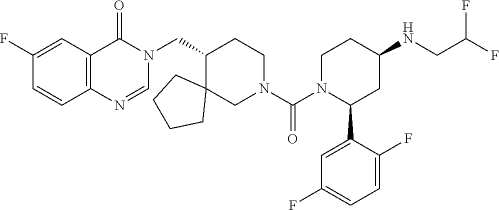 [SMILES: O=C(N1CC[C@@H](Cn2cnc3ccc(F)cc3c2=O)C2(CCCC2)C1)N1CC[C@@H](NCC(F)F)C[C@H]1c1cc(F)ccc1F] | 3-(((R)-7-((2S,4R)-4- ((2,2- Difluoroethyl)amino)-2- (2,5- difluorophenyl)piperidine- 1-carbonyl)-7- azaspiro[4.5]decan-10- yl)methyl)-6- fluoroquinazolin-4(3H)- one | 8.37 (s, 1H), 7.82 (dd, 1H), 7.77 (dd, 1H), 7.72 (td, 1H), 7.13 (td, 1H), 7.06-7.00 (m, 1H), 7.00-6.94 (m, 1H), 5.92 (t, 1H), 4.24 (d, 1H), 4.16 (dd, 1H), 3.88-3.73 (m, 2H), 3.40 (d, 1H), 3.30-3.25 (m, 2H), 2.89 (t, 2H), 2.74 (d, 1H), 2.68-2.58 (m, 2H), 2.14-1.73 (m, 5H), 1.72-1.47 (m, 4H), 1.45-1.17 (m, 7H). | (Method B) R_{T} = 0.95 min, m/z 618 [M + H]⁺ |
| 158 | 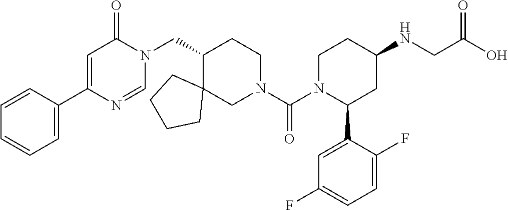 [SMILES: O=C(O)CN[C@@H]1CCN(C(=O)N2CC[C@@H](Cn3cnc(-c4ccccc4)cc3=O)C3(CCCC3)C2)[C@H](c2cc(F)ccc2F)C1] | ((2S,4R)-2-(2,5- Difluorophenyl)-1-((R)- 10-((6-oxo-4- phenylpyrimidin-1(6H)- yl)methyl)-7- azaspiro[4.5]decane-7- carbonyl)piperidin-4- yl)glycine | 8.54 (s, 1H), 8.06 (dd, J = 6.7, 3.0 Hz, 2H), 7.49 (dd, J = 5.1, 1.9 Hz, 3H), 7.16 (td, J = 9.3, 4.5 Hz, 1H), 7.06 (ddd, J = 8.9, 7.4, 3.5 Hz, 1H), 7.00-6.93 (m, 2H), 4.29-4.21 (m, 1H), 4.11 (dd, J = 13.1, 3.0 Hz, 1H), 3.84 (dt, J = 12.5, 3.8 Hz, 1H), 3.70 (d, J = 11.1 Hz, 1H), 3.46-3.36 (m, 3H, obscured by HDO signal), 3.18- 3.09 (m, 3H), 3.02 (d, J = 11.5 Hz, 1H), 2.85 (s, 1H), 2.75 (d, J = 12.9 Hz, 1H), 2.70-2.64 (m, 1H), 2.01 (d, J = 10.9 Hz, 2H), 1.86 (td, J = 10.6, 5.3 Hz, 1H), 1.75 (s, 1H), 1.72-1.43 (m, 6H), 1.42- 1.12 (m, 5H). | (Method B) R_{T} = 1.02 min, m/z 620 [M + H]⁺ |
| 159 | 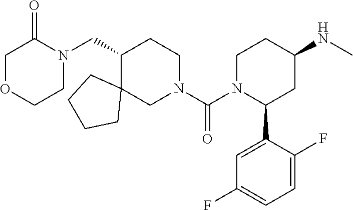 [SMILES: CN[C@@H]1CCN(C(=O)N2CC[C@@H](CN3CCOCC3=O)C3(CCCC3)C2)[C@H](c2cc(F)ccc2F)C1] | 4-(((R)-7-((2S,4R)-2-(2,5- Difluorophenyl)-4- (methylamino)piperidine- 1-carbonyl)-7- azaspiro[4.5]decan-10- yl)methyl)morpholin-3- one | 7.13 (td, J = 9.2, 4.5 Hz, 1H), 7.00 (dddd, J = 14.9, 8.9, 6.5, 3.4 Hz, 2H), 4.24 (dd, J = 11.8, 2.5 Hz, 1H), 4.02 (s, 2H), 3.85- 3.73 (m, 3H), 3.67 (s, 1H), 3.37 (dt, J = 12.3, 5.1 Hz, 1H), 3.29-3.17 (m, 3H), 3.16-2.96 (m, 2H), 2.81 (d, J = 12.8 Hz, 1H), 2.69-2.60 (m, 1H), 2.45-2.38 (m, 1H), 2.24 (s, 3H), 1.98-1.87 (m, 2H), 1.80-1.69 (m, 1H), 1.67-1.29 (m, 8H), 1.29-1.08 (m, 5H). | (Method B) R_{T} = 0.72 min, m/z 505 [M + H]⁺ |
| 160 | 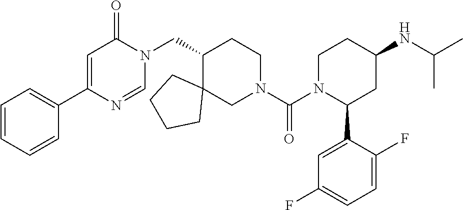 [SMILES: CC(C)N[C@@H]1CCN(C(=O)N2CC[C@@H](Cn3cnc(-c4ccccc4)cc3=O)C3(CCCC3)C2)[C@H](c2cc(F)ccc2F)C1] | 3-(((R)-7-((2S,4R)-2-(2,5- Difluorophenyl)-4- (isopropylamino)piperidine- 1-carbonyl)-7- azaspiro[4.5]decan-10- yl)methyl)-6- phenylpyrimidin-4(3H)- one | 8.55 (s, 1H), 8.09-8.03 (m, 2H), 7.49 (dd, J = 5.1, 1.9 Hz, 3H), 7.14 (td, J = 9.3, 4.5 Hz, 1H), 7.02 (tt, J = 8.1, 3.4 Hz, 1H), 7.00-6.93 (m, 2H), 4.27 (d, J = 12.2 Hz, 1H), 4.11 (dd, J = 13.1, 3.1 Hz, 1H), 3.85 (dd, J = 11.3, 6.8 Hz, 1H), 3.72 (t, J = 12.0 Hz, 1H), 3.41 (d, J = 12.9 Hz, 1H), 3.34-3.24 (m, 2H, obscured by HDO signal), 2.87 (s, 2H), 2.75 (d, J = 12.9 Hz, 1H), 2.69 (d, J = 12.3 Hz, 1H), 1.97- 1.83 (m, 3H), 1.78 (s, 1H), 1.72-1.45 (m, 4H), 1.45-1.13 (m, 8H), 0.95 (t, J = 6.8 Hz, 6H). | (Method B) R_{T} = 0.96 min, m/z 604 [M + H]⁺ |
| 161 | 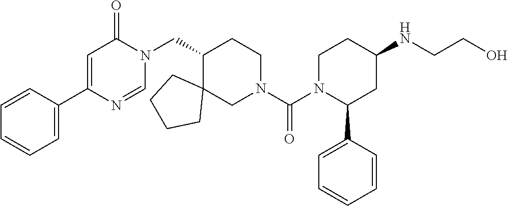 [SMILES: O=C(N1CC[C@@H](Cn2cnc(-c3ccccc3)cc2=O)C2(CCCC2)C1)N1CC[C@@H](NCCO)C[C@H]1c1ccccc1] | 3-(((R)-7-((2S,4R)-4-((2- Hydroxyethyl)amino)-2- phenylpiperidine-1- carbonyl)-7- azaspiro[4.5]decan-10- yl)methyl)-6- phenylpyrimidin-4(3H)- one | 8.55 (s, 1H), 8.06 (dd, J = 6.6, 3.0 Hz, 2H), 7.49 (dd, J = 5.1, 1.9 Hz, 3H), 7.25- 7.16 (m, 4H), 7.16-7.09 (m, 1H), 6.97 (s, 1H), 4.41 (t, J = 5.3 Hz, 1H), 4.10 (dd, J = 13.0, 3.0 Hz, 1H), 3.95 (dd, J = 11.7, 3.1 Hz, 1H), 3.92-3.83 (m, 1H), 3.70 (t, J = 12.0 Hz, 1H), 3.44 (d, J = 13.0 Hz, 1H), 3.40 (q, J = 5.6 Hz, 2H), 3.25 (dt, J = 12.2, 3.7 Hz, 1H), 2.83 (s, 1H), 2.72- 2.62 (m, 2H), 2.58 (t, J = 5.9 Hz, 3H), 1.95-1.81 (m, 3H), 1.77 (s, 1H), 1.72- 1.42 (m, 4H), 1.42-1.12 (m, 7H). [OH signal not observed]. | (Method B) R_{T} = 0.90 min, m/z 570 [M + H]⁺ |
| 162 | 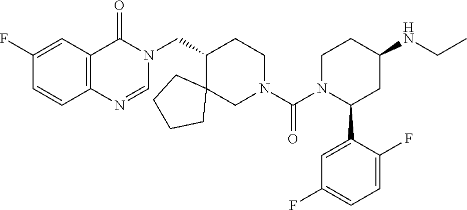 [SMILES: CCN[C@@H]1CCN(C(=O)N2CC[C@@H](Cn3cnc4ccc(F)cc4c3=O)C3(CCCC3)C2)[C@H](c2cc(F)ccc2F)C1] | 3-(((R)-7-((2S,4R)-2-(2,5- Difluorophenyl)-4- (ethylamino)piperidine-1- carbonyl)-7- azaspiro[4.5]decan-10- yl)methyl)-6- fluoroquinazolin-4(3H)- one | 8.37 (s, 1H), 7.82 (dd, 1H), 7.79-7.69 (m, 2H), 7.13 (td, 1H), 7.06-6.92 (m, 2H), 4.25 (d, 1H), 4.16 (dd, 1H), 3.90-3.71 (m, 2H), 3.40 (d, 1H), 3.30-3.25 (m, 2H), 2.82 (br s, 1H), 2.73 (d, 1H), 2.66 (t, 1H), 2.61- 2.52 (m, 3H), 1.95-1.85 (m, 3H), 1.84- 1.74 (m, 1H), 1.71-1.44 (m, 4H), 1.43- 1.15 (m, 7H), 0.97 (t, 3H). | (Method B) R_{T} = 0.93 min, m/z 582 [M + H]⁺ |
| 163 | 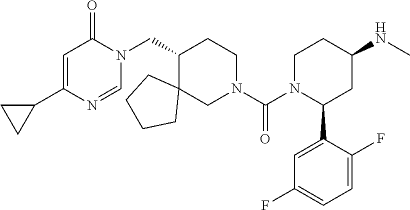 [SMILES: CN[C@@H]1CCN(C(=O)N2CC[C@@H](Cn3cnc(C4CC4)cc3=O)C3(CCCC3)C2)[C@H](c2cc(F)ccc2F)C1] | 6-Cyclopropyl-3-(((R)-7- ((2S,4R)-2-(2,5- difluorophenyl)-4- (methylamino)piperidine- 1-carbonyl)-7- azaspiro[4.5]decan-10- yl)methyl)pyrimidin- 4(3H)-one | 8.28 (s, 1H), 7.13 (td, 1H), 7.06-6.91 (m, 2H), 6.31 (s, 1H), 4.25 (d, 1H), 4.01 (dd, 1H), 3.89-3.77 (m, 1H), 3.61 (t, 1H), 3.41- 3.34 (m, 1H), 3.29-3.24 (m, 1H), 2.83 (br s, 1H), 2.71 (d, 1H), 2.68-2.60 (m, 1H), 2.53-2.52 (m, 1H), 2.47-2.39 (m, 1H), 2.25 (s, 3H), 1.98-1.82 (m, 3H), 1.82-1.68 (m, 2H), 1.68-1.40 (m, 4H), 1.40-1.27 (m, 2H), 1.27-1.06 (m, 5H), 0.97-0.84 (m, 4H). | (Method B) R_{T} = 0.83 min, m/z 540 [M + H]⁺ |
| 164 | 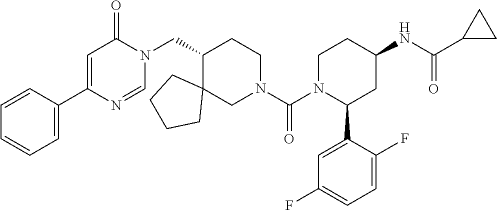 [SMILES: O=C(N[C@@H]1CCN(C(=O)N2CC[C@@H](Cn3cnc(-c4ccccc4)cc3=O)C3(CCCC3)C2)[C@H](c2cc(F)ccc2F)C1)C1CC1] | N-((2S,4R)-2-(2,5- Difluorophenyl)-1-((R)- 10-((6-oxo-4- phenylpyrimidin-1(6H)- yl)methyl)-7- azaspiro[4.5]decane-7- carbonyl)piperidin-4- yl)cyclopropanecarbox- amide | 8.55 (s, 1H), 8.10-8.04 (m, 2H), 8.01 (d, J = 7.5 Hz, 1H), 7.49 (dd, J = 5.1, 1.9 Hz, 3H), 7.15 (td, J = 9.3, 4.5 Hz, 1H), 7.08- 7.00 (m, 1H), 7.00-6.93 (m, 2H), 4.27 (dt, J = 11.6, 2.3 Hz, 1H), 4.11 (dd, J = 13.1, 3.0 Hz, 1H), 3.90-3.82 (m, 1H), 3.73 (dtt, J = 15.8, 8.0, 4.3 Hz, 2H), 3.40 (d, J = 12.7 Hz, 1H), 3.29 (s, 1H, obscured by HDO), 2.88 (s, 1H), 2.79- 2.67 (m, 2H), 1.88 (dq, J = 10.7, 7.0, 5.3 Hz, 3H), 1.78 (s, 1H), 1.72-1.43 (m, 6H), 1.43-1.15 (m, 6H), 0.62 (ddt, J = 13.7, 9.0, 4.7 Hz, 4H). | (Method B) R_{T} = 1.39 min, m/z 630 [M + H]⁺ |
| 165 | 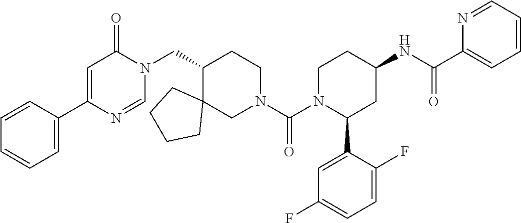 [SMILES: O=C(N[C@@H]1CCN(C(=O)N2CC[C@@H](Cn3cnc(-c4ccccc4)cc3=O)C3(CCCC3)C2)[C@H](c2cc(F)ccc2F)C1)c1ccccn1] | N-((2S,4R)-2-(2,5- Difluorophenyl)-1-((R)- 10-((6-oxo-4- phenylpyrimidin-1(6H)- yl)methyl)-7- azaspiro[4.5]decane-7- carbonyl)piperidin-4- yl)picolinamide | 8.68-8.58 (m, 2H), 8.55 (s, 1H), 8.07 (dd, J = 6.8, 3.1 Hz, 2H), 8.04-7.96 (m, 2H), 7.59 (ddd, J = 7.4, 4.7, 1.5 Hz, 1H), 7.49 (dd, J = 5.0, 1.9 Hz, 3H), 7.15 (td, J = 9.2, 4.5 Hz, 1H), 7.02 (dddd, J = 12.4, 9.0, 6.3, 3.3 Hz, 2H), 6.97 (s, 1H), 4.36 (dd, J = 11.6, 2.6 Hz, 1H), 4.12 (dd, J = 13.0, 3.0 Hz, 1H), 4.09-3.97 (m, 1H), 3.88 (d, J = 13.1 Hz, 1H), 3.72 (t, J = 12.6 Hz, 1H), 3.45 (d, J = 11.3 Hz, 1H), 3.35 (d, J = 12.6 Hz, 1H), 2.77 (d, J = 13.4 Hz, 3H), 2.00-1.46 (m, 10H), 1.46-1.26 (m, 3H), 1.22 (dt, J = 13.6, 7.1 Hz, 2H). | (Method B) R_{T} = 1.51 min, m/z 667 [M + H]⁺ |
| 166 | 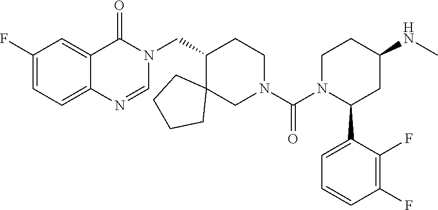 [SMILES: CN[C@@H]1CCN(C(=O)N2CC[C@@H](Cn3cnc4ccc(F)cc4c3=O)C3(CCCC3)C2)[C@H](c2cccc(F)c2F)C1] | 3-(((R)-7-((2S,4R)-2-(2,3- Difluorophenyl)-4- (methylamino)piperidine- 1-carbonyl)-7- azaspiro[4.5]decan-10- yl)methyl)-6- fluoroquinazolin-4(3H)- one | 8.37 (s, 1H), 7.82 (dd, J = 8.7, 2.9 Hz, 1H), 7.79-7.69 (m, 2H), 7.21 (dtd, J = 10.0, 7.7, 1.8 Hz, 1H), 7.06 (ddd, J = 15.3, 12.5, 7.6 Hz, 2H), 4.30 (dd, J = 12.0, 2.9 Hz, 1H), 4.15 (dd, J = 13.3, 3.0 Hz, 1H), 3.87-3.71 (m, 2H), 3.39 (d, J = 13.0 Hz, 1H), 3.35-3.25 (m, 2H, signal obscured by HDO), 2.83 (s, 1H), 2.69 (dt, J = 12.5, 5.5 Hz, 2H), 2.59 (s, 1H), 2.30 (s, 3H), 1.96 (d, J = 12.4 Hz, 2H), 1.86 (dq, J = 6.9, 4.9, 3.4 Hz, 1H), 1.79 (s, 1H), 1.66 (s, 2H), 1.59-1.46 (m, 2H), 1.46-1.13 (m, 7H). | (Method B) R_{T} = 0.90 min, m/z 568 [M + H]⁺ |
| 167 | 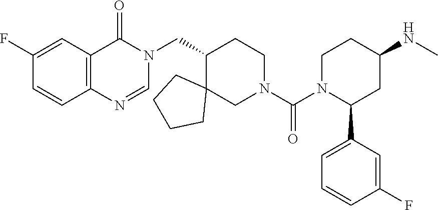 [SMILES: CN[C@@H]1CCN(C(=O)N2CC[C@@H](Cn3cnc4ccc(F)cc4c3=O)C3(CCCC3)C2)[C@H](c2cccc(F)c2)C1] | 6-Fluoro-3-(((R)-7- ((2S,4R)-2-(3- fluorophenyl)-4- (methylamino)piperidine- 1-carbonyl)-7- azaspiro[4.5]decan-10- yl)methyl)quinazolin- 4(3H)-one | 8.37 (s, 1H), 7.82 (dd, J = 8.7, 2.9 Hz, 1H), 7.79-7.69 (m, 2H), 7.29-7.23 (m, 1H), 7.04 (dt, J = 7.7, 1.2 Hz, 1H), 6.99- 6.93 (m, 2H), 4.16 (dd, J = 13.3, 3.0 Hz, 1H), 3.99 (dd, J = 11.8, 3.0 Hz, 1H), 3.85 (dd, J = 13.4, 4.5 Hz, 1H), 3.78 (t, J = 12.1 Hz, 1H), 3.43 (d, J = 12.9 Hz, 1H), 3.29-3.21 (m, 2H, obscured by HDO signal), 2.82 (s, 1H), 2.73-2.62 (m, 2H), 2.47-2.42 (m, 1H, obscure by DMSO signal), 2.26 (s, 3H), 1.96-1.84 (m, 3H), 1.80 (s, 1H), 1.72-1.60 (m, 2H), 1.60- 1.45 (m, 2H), 1.41-1.25 (m, 4H), 1.25- 1.13 (m, 3H). | (Method B) R_{T} = 0.89 min, m/z 550 [M + H]⁺ |
| 168 | 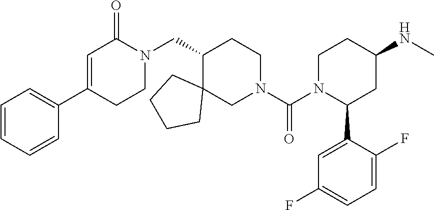 [SMILES: CN[C@@H]1CCN(C(=O)N2CC[C@@H](CN3CCC(c4ccccc4)=CC3=O)C3(CCCC3)C2)[C@H](c2cc(F)ccc2F)C1] | 1-(((R)-7-((2S,4R)-2-(2,5- Difluorophenyl)-4- (methylamino)piperidine- 1-carbonyl)-7- azaspiro[4.5]decan-10- yl)methyl)-4-phenyl-5,6- dihydropyridin-2(1H)-one | 7.62 (d, 2H), 7.51-7.35 (m, 3H), 7.13 (td, 1H), 7.07-6.95 (m, 2H), 6.20 (s, 1H), 4.24 (d, 1H), 3.84-3.74 (m, 1H), 3.67-3.56 (m, 1H), 3.55-3.43 (m, 2H), 3.28-3.19 (m, 2H), 3.19-3.01 (m, 2H), 2.87-2.72 (m, 3H), 2.69-2.61 (m, 1H), 2.47-2.40 (m, 2H), 2.25 (s, 3H), 1.92 (d, 2H), 1.77-1.68 (m, 1H), 1.65-1.47 (m, 5H), 1.47-1.32 (m, 2H), 1.32-1.10 (m, 5H). | (Method B) R_{T} = 0.99 min, m/z 577 [M + H]⁺ |
| 169 | 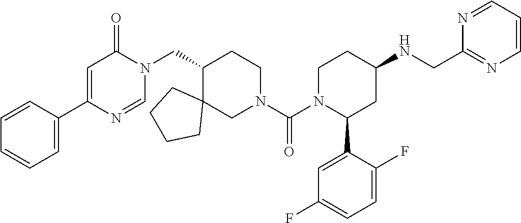 [SMILES: O=C(N1CC[C@@H](Cn2cnc(-c3ccccc3)cc2=O)C2(CCCC2)C1)N1CC[C@@H](NCc2ncccn2)C[C@H]1c1cc(F)ccc1F] | 3-(((R)-7-((2S,4R)-2-(2,5- Difluorophenyl)-4- ((pyrimidin-2- ylmethyl)amino)piperidine- 1-carbonyl)-7- azaspiro[4.5]decan-10- yl)methyl)-6- phenylpyrimidin-4(3H)- one | 8.85 (d, J = 4.9 Hz, 2H), 8.55 (s, 1H), 8.06 (dd, J = 6.7, 3.0 Hz, 2H), 7.49 (dd, J = 5.2, 2.0 Hz, 4H), 7.17 (td, J = 9.3, 4.5 Hz, 1H), 7.07 (tt, J = 8.3, 3.5 Hz, 1H), 7.03-6.95 (m, 2H), 4.30 (d, J = 11.6 Hz, 3H), 4.11 (dd, J = 13.1, 3.0 Hz, 1H), 3.85 (d, J = 12.9 Hz, 1H), 3.73 (t, J = 11.2 Hz, 1H), 3.45-3.37 (m, 1H), 3.37-3.31 (m, 1H), 2.87 (s, 1H), 2.77 (d, J = 12.9 Hz, 1H), 2.69 (t, J = 12.3 Hz, 1H), 2.46-2.41 (m, 1H, obscured by DMSO signal), 2.12 (s, 2H), 1.88 (t, J = 10.7 Hz, 1H), 1.64 (q, J = 43.9, 35.7 Hz, 7H), 1.42-1.13 (m, 6H). | (Method B) R_{T} = 0.94 min, m/z 654 [M + H]⁺ |
| 170 | 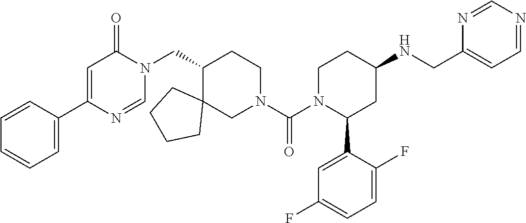 [SMILES: O=C(N1CC[C@@H](Cn2cnc(-c3ccccc3)cc2=O)C2(CCCC2)C1)N1CC[C@@H](NCc2ccncn2)C[C@H]1c1cc(F)ccc1F] | 3-(((R)-7-((2S,4R)-2-(2,5- Difluorophenyl)-4- ((pyrimidin-4- ylmethyl)amino)piperidine- 1-carbonyl)-7- azaspiro[4.5]decan-10- yl)methyl)-6- phenylpyrimidin-4(3H)- one | 9.17 (s, 1H), 8.80 (s, 1H), 8.55 (s, 1H), 8.08-8.03 (m, 2H), 7.59 (dd, J = 5.2, 1.4 Hz, 1H), 7.49 (dd, J = 5.0, 1.9 Hz, 3H), 7.17 (td, J = 9.3, 4.4 Hz, 1H), 7.07 (s, 1H), 7.02-6.95 (m, 2H), 4.38-3.96 (m, 3H), 3.85 (d, J = 13.2 Hz, 1H), 3.73 (s, 1H), 3.39 (s, 1H), 3.34 (s, 1H), 2.86 (s, 1H), 2.77 (d, J = 12.9 Hz, 1H), 2.74- 2.65 (m, 1H), 2.53-2.51 (m, 2H), 2.09 (s, 2H), 1.92-1.84 (m, 1H), 1.78 (s, 1H), 1.73-1.43 (m, 6H), 1.43-1.13 (m, 6H). | (Method B) R_{T} = 0.94 min, m/z 654 [M + H]⁺ |
| 171 | 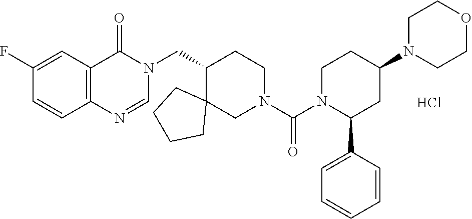 [SMILES: O=C(N1CC[C@@H](Cn2cnc3ccc(F)cc3c2=O)C2(CCCC2)C1)N1CC[C@@H](N2CCOCC2)C[C@H]1c1ccccc1 ; Cl] | 6-Fluoro-3-(((R)-7- ((2S,4R)-4-morpholino-2- phenylpiperidine-1- carbonyl)-7- azaspiro[4.5]decan-10- yl)methyl)quinazolin- 4(3H)-one hydrochloride | 10.22 (s, 1H), 8.36 (s, 1H), 7.82 (dd, J = 8.6, 2.9 Hz, 1H), 7.79-7.70 (m, 2H), 7.31-7.14 (m, 5H), 4.14 (dd, J = 13.3, 3.0 Hz, 1H), 4.05-3.90 (m, 3H), 3.90- 3.76 (m, 2H), 3.71 (td, J = 12.1, 8.1 Hz, 2H), 3.47-3.40 (m, 4H, obscured by HDO signal), 3.40-3.32 (m, 1H), 3.09 (p, J = 10.9 Hz, 2H), 2.86 (s, 1H), 2.70 (t, J = 12.3 Hz, 2H), 2.19 (d, J = 11.9 Hz, 2H), 1.93-1.44 (m, 8H), 1.40-1.12 (m, 5H). | (Method B) R_{T} = 0.89 min, m/z 588 [M + H]⁺ |
| 172 | 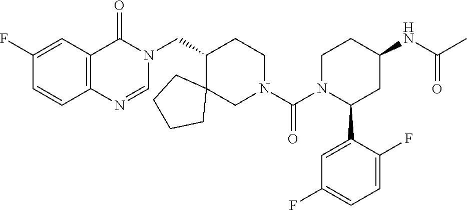 [SMILES: CC(=O)N[C@@H]1CCN(C(=O)N2CC[C@@H](Cn3cnc4ccc(F)cc4c3=O)C3(CCCC3)C2)[C@H](c2cc(F)ccc2F)C1] | N-((2S,4R)-2-(2,5- Difluorophenyl)-1-((R)- 10-((6-fluoro-4- oxoquinazolin-3(4H)- yl)methyl)-7- azaspiro[4.5]decane-7- carbonyl)piperidin-4- yl)acetamide | 8.37 (s, 1H), 7.86-7.70 (m, 4H), 7.15 (td, J = 9.3, 4.5 Hz, 1H), 7.03 (ddd, J = 8.8, 7.4, 3.5 Hz, 1H), 6.95 (ddd, J = 9.0, 5.5, 3.3 Hz, 1H), 4.31-4.24 (m, 1H), 4.16 (dd, J = 13.4, 2.9 Hz, 1H), 3.89-3.75 (m, 2H), 3.70 (dtt, J = 11.6, 8.1, 3.9 Hz, 1H), 3.40 (d, J = 12.8 Hz, 1H), 3.28 (d, J = 3.5 Hz, 1H), 2.85 (s, 1H), 2.79-2.67 (m, 2H), 1.89 (td, J = 10.4, 3.0 Hz, 3H), 1.81 (s, 1H), 1.76 (s, 3H), 1.65 (d, J = 18.1 Hz, 2H), 1.60-1.45 (m, 3H), 1.44-1.26 (m, 4H), 1.22 (dt, J = 13.9, 7.1 Hz, 2H). | (Method B) R_{T} = 1.26 min, m/z 596 [M + H]⁺ |
| 173 | 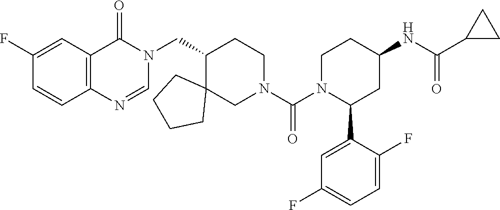 [SMILES: O=C(N[C@@H]1CCN(C(=O)N2CC[C@@H](Cn3cnc4ccc(F)cc4c3=O)C3(CCCC3)C2)[C@H](c2cc(F)ccc2F)C1)C1CC1] | N-((2S,4R)-2-(2,5- Difluorophenyl)-1-((R)- 10-((6-fluoro-4- oxoquinazolin-3(4H)- yl)methyl)-7- azaspiro[4.5]decane-7- carbonyl)piperidin-4- yl)cyclopropanecarboxa mide | 8.37 (s, 1H), 8.01 (d, J = 7.5 Hz, 1H), 7.83 (dd, J = 8.6, 2.9 Hz, 1H), 7.79-7.69 (m, 2H), 7.15 (td, J = 9.3, 4.5 Hz, 1H), 7.04 (tt, J = 8.3, 3.5 Hz, 1H), 6.96 (ddd, J = 9.0, 5.5, 3.3 Hz, 1H), 4.30-4.24 (m, 1H), 4.16 (dd, J = 13.3, 2.9 Hz, 1H), 3.89- 3.67 (m, 3H), 3.41 (d, J = 13.5 Hz, 2H), 2.85 (s, 1H), 2.79-2.65 (m, 2H), 1.93- 1.85 (m, 3H), 1.81 (s, 1H), 1.74-1.43 (m, 6H), 1.43-1.15 (m, 6H), 0.68-0.55 (m, 4H). | (Method B) R_{T} = 1.37 min, m/z 622 [M + H]⁺ |
| 174 | 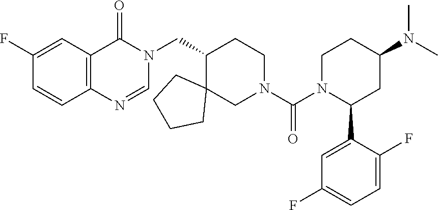 [SMILES: CN(C)[C@@H]1CCN(C(=O)N2CC[C@@H](Cn3cnc4ccc(F)cc4c3=O)C3(CCCC3)C2)[C@H](c2cc(F)ccc2F)C1] | 3-(((R)-7-((2S,4R)-2-(2,5- Difluorophenyl)-4- (dimethylamino)piperidine- 1-carbonyl)-7- azaspiro[4.5]decan-10- yl)methyl)-6- fluoroquinazolin-4(3H)- one | 8.37 (s, 1H), 7.82 (dd, J = 8.7, 2.9 Hz, 1H), 7.79-7.69 (m, 2H), 7.13 (td, J = 9.7, 4.6 Hz, 1H), 7.02 (ddd, J = 7.9, 6.1, 3.1 Hz, 2H), 4.27-4.21 (m, 1H), 4.15 (dd, J = 13.3, 3.0 Hz, 1H), 3.89-3.76 (m, 2H), 3.39 (d, J = 12.9 Hz, 1H), 3.35-3.32 (m, 1H), 2.86 (s, 1H), 2.75 (d, J = 12.9 Hz, 1H), 2.66 (d, J = 11.5 Hz, 1H), 2.32 (dd, J = 14.0, 5.6 Hz, 1H), 2.16 (s, 6H), 1.92- 1.73 (m, 4H), 1.71-1.44 (m, 5H), 1.43- 1.15 (m, 6H). | (Method B) R_{T} = 0.91 min, m/z 582 [M + H]⁺ |
| 175 | 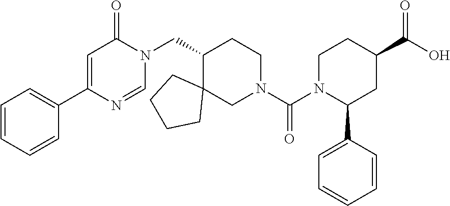 [SMILES: O=C(O)[C@@H]1CCN(C(=O)N2CC[C@@H](Cn3cnc(-c4ccccc4)cc3=O)C3(CCCC3)C2)[C@H](c2ccccc2)C1] | (2S,4R)-1-((R)-10-((6- Oxo-4-phenylpyrimidin- 1(6H)-yl)methyl)-7- azaspiro[4.5]decane-7- carbonyl)-2- phenylpiperidine-4- carboxylic acid | 8.55 (s, 1H), 8.09-8.02 (m, 2H), 7.52-7.46 (m, 3H), 7.25-7.17 (m, 4H), 7.17-7.11 (m, 1H), 6.97 (s, 1H), 4.10 (dd, 1H), 3.95 (dd, 1H), 3.92-3.84 (m, 1H), 3.70 (t, 1H), 3.44 (d 1H), 2.91-2.77 (m, 1H), 2.72-2.61 (m, 2H), 2.43-2.36 (m, 1H), 1.97-1.81 (m, 3H), 1.81-1.72 (m, 1H), 1.72-1.59 (m, 3H), 1.59-1.42 (m, 3H), 1.39-1.13 (m, 6H). [OH not observed] | (Method B) R_{T} = 1.31 min, m/z 555 [M + H]⁺ |
| 176 | 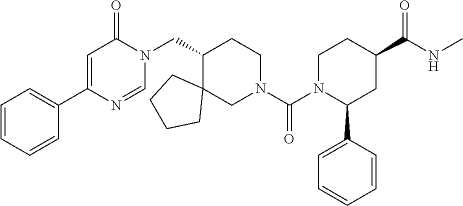 [SMILES: CNC(=O)[C@@H]1CCN(C(=O)N2CC[C@@H](Cn3cnc(-c4ccccc4)cc3=O)C3(CCCC3)C2)[C@H](c2ccccc2)C1] | (2S,4R)-N-Methyl-1-((R)- 10-((6-oxo-4- phenylpyrimidin-1(6H)- yl)methyl)-7- azaspiro[4.5]decane-7- carbonyl)-2- phenylpiperidine-4- carboxamide | 8.55 (s, 1H), 8.10-8.02 (m, 2H), 7.76-7.68 (m, 1H), 7.53-7.44 (m, 3H), 7.27-7.17 (m, 4H), 7.17-7.12 (m, 1H), 6.97 (s, 1H), 4.10 (dd, 1H), 3.93 (dd, 1H), 3.91-3.85 (m, 1H), 3.70 (t, 1H), 3.45 (d 1H), 3.29-3.26 (m, 1H), 2.89-2.77 (m, 1H), 2.70-2.61 (m, 2H), 2.54-2.52 (d, 3H), 2.37-2.28 (m, 1H), 1.88-1.59 (m, 7H), 1.59-1.42 (m, 3H), 1.37-1.25 (m, 3H), 1.25-1.13 (m, 2H). | (Method B) R_{T} = 1.27 min, m/z 568 [M + H]⁺ |
| 177 | 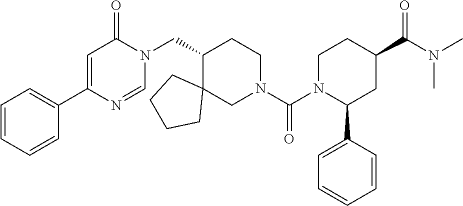 [SMILES: CN(C)C(=O)[C@@H]1CCN(C(=O)N2CC[C@@H](Cn3cnc(-c4ccccc4)cc3=O)C3(CCCC3)C2)[C@H](c2ccccc2)C1] | (2S,4R)-N,N-Dimethyl-1- ((R)-10-((6-oxo-4- phenylpyrimidin-1(6H)- yl)methyl)-7- azaspiro[4.5]decane-7- carbonyl)-2- phenylpiperidine-4- carboxamide | 8.55 (s, 1H), 8.11-8.02 (m, 2H), 7.54-7.45 (m, 3H), 7.25-7.17 (m, 4H), 7.17-7.12 (m, 1H), 6.97 (s, 1H), 4.10 (dd, 1H), 4.04 (dd, 1H), 3.92-3.83 (m, 1H), 3.71 (t, 1H), 3.44 (d 1H), 3.29-3.24 (m, 1H), 3.03 (s, 3H), 2.92-2.81 (m, 2H), 2.78 (s, 3H), 2.77-2.72 (m, 1H), 2.70-2.64 (d, 1H), 1.89-1.81 (m, 1H), 1.81-1.71 (m, 3H), 1.71-1.59 (m, 3H), 1.59-1.43 (m, 3H), 1.37-1.25 (m, 3H), 1.25-1.14 (m, 2H). | (Method B) R_{T} = 1.34 min, m/z 582 [M + H]⁺ |
| 178 | 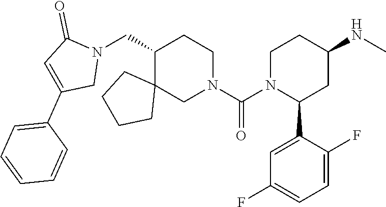 [SMILES: CN[C@@H]1CCN(C(=O)N2CC[C@@H](CN3CC(c4ccccc4)=CC3=O)C3(CCCC3)C2)[C@H](c2cc(F)ccc2F)C1] | 1-(((R)-7-((2S,4R)-2-(2,5- Difluorophenyl)-4- (methylamino)piperidine- 1-carbonyl)-7- azaspiro[4.5]decan-10- yl)methyl)-4-phenyl-1,5- dihydro-2H-pyrrol-2-one | 8.30 (s, 1H), 7.54-7.49 (m, 2H), 7.45 (dd, J = 8.4, 6.8 Hz, 2H), 7.41-7.36 (m, 1H), 7.11 (td, J = 9.3, 4.5 Hz, 1H), 7.03- 6.96 (m, 1H), 6.93 (ddd, J = 9.0, 5.4, 3.2 Hz, 1H), 4.27-4.11 (m, 3H), 3.57 (d, J = 12.3 Hz, 1H), 3.29-3.24 (m, 1H), 3.22- 3.13 (m, 1H), 2.75 (s, 1H), 2.67 (d, J = 12.8 Hz, 1H), 2.62-2.55 (m, 1H), 2.47- 2.38 (m, 3H), 2.35-2.28 (m, 1H), 2.24 (s, 3H), 1.89 (dd, J = 12.9, 3.8 Hz, 2H), 1.63- 1.35 (m, 6H), 1.35-1.02 (m, 6H), 0.88 (s, 1H). | (Method K) R_{T} = 0.85 min, m/z 563 [M + H]⁺ |
| 179 | 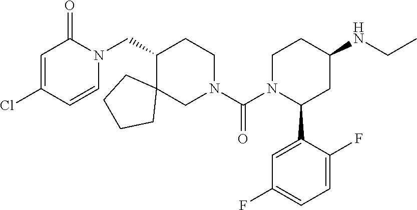 [SMILES: CCN[C@@H]1CCN(C(=O)N2CC[C@@H](Cn3ccc(Cl)cc3=O)C3(CCCC3)C2)[C@H](c2cc(F)ccc2F)C1] | 4-Chloro-1-(((R)-7- ((2S,4R)-2-(2,5- difluorophenyl)-4- (ethylamino)piperidine-1- carbonyl)-7- azaspiro[4.5]decan-10- yl)methyl)pyridin-2(1H)- one | 7.73 (d, 1H), 7.13 (td, 1H), 7.05-6.93 (m, 2H), 6.52 (d, 1H), 6.35 (dd, 1H), 4.25 (d, 1H), 4.04 (dd, 1H), 3.82 (dt, 1H), 3.68 (t, 1H), 3.39-3.33 (m, 1H), 3.22-3.29 (m, 2H), 2.85 (br s, 1H), 2.73 (d, 1H), 2.65 (t, 1H), 2.62-2.52 (m, 3H), 1.95-1.87 (m, 2H), 1.83-1.68 (m, 2H), 1.68-1.42 (m, 4H), 1.42-1.10 (m, 7H), 0.97 (t, 3H). | (Method B) R_{T} = 0.90 min, m/z 547 [M + H]⁺ |
| 180 | 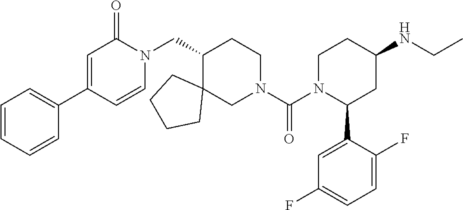 [SMILES: CCN[C@@H]1CCN(C(=O)N2CC[C@@H](Cn3ccc(-c4ccccc4)cc3=O)C3(CCCC3)C2)[C@H](c2cc(F)ccc2F)C1] | 1-(((R)-7-((2S,4R)-2-(2,5- Difluorophenyl)-4- (ethylamino)piperidine-1- carbonyl)-7- azaspiro[4.5]decan-10- yl)methyl)-4- phenylpyridin-2(1H)-one | 7.76-7.68 (m, 3H), 7.52-7.42 (m, 3H), 7.13 (td, 1H), 7.06-6.94 (m, 2H), 6.66 (d, 1H), 6.57 (dd, 1H), 4.26 (d, 1H), 4.09 (d, 1H), 3.85 (dt, 1H), 3.73 (t, 1H), 3.44-3.34 (m, 1H), 3.30-3.25 (m, 2H), 2.88 (br s, 1H), 2.76 (d, 1H), 2.66 (t, 1H), 2.62-2.53 (m, 3H), 1.92 (d, 2H), 1.89-1.82 (m, 1H), 1.82-1.72 (m, 1H), 1.70-1.44 (m, 4H), 1.44-1.30 (m, 3H), 1.30-1.15 (m, 4H), 0.98 (t, 3H). | (Method B) R_{T} = 1.00 min, m/z 589 [M + H]⁺ |
| 181 | 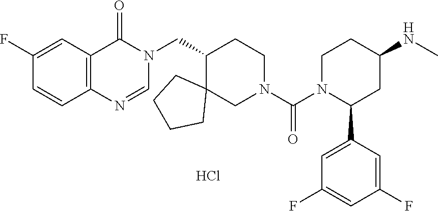 | 3-(((R)-7-((2S,4R)-4- Amino-2-(3,5- difluorophenyl)piperidine- 1-carbonyl)-7- azaspiro[4.5]decan-10- yl)methyl)-6- fluoroquinazolin-4(3H)- one hydrochloride | 8.36 (s, 1H), 8.00 (br s, 3H), 7.82 (dd, 1H), 7.79-7.69 (m, 2H), 7.06 (tt, 1H), 6.94-3.83 (m, 2H), 4.15 (d, 1H), 4.08 (dd, 1H), 3.90-3.74 (m, 2H), 3.48-3.36 (m, 1H), 3.30-3.27 (m, 1H), 3.27-3.19 (m, 1H), 2.84 (br s, 1H), 2.76 (d, 1H), 2.70 (t, 1H), 2.02 (d, 2H), 1.94-1.86 (m, 1H), 1.86-1.75 (m, 1H), 1.75-1.61 (m, 3H), 1.61-1.46 (m, 3H), 1.44-1.14 (m, 5H). | (Method B) R_{T} = 0.95 min, m/z 554 [M + H]⁺ |
| 182 | 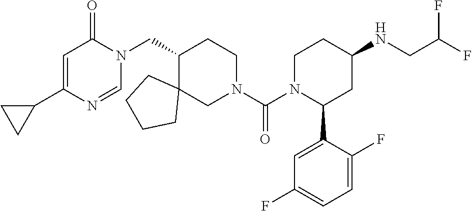 [SMILES: O=C(N1CC[C@@H](Cn2cnc(C3CC3)cc2=O)C2(CCCC2)C1)N1CC[C@@H](NCC(F)F)C[C@H]1c1cc(F)ccc1F] | 6-Cyclopropyl-3-(((R)-7- ((2S,4R)-4-((2,2- difluoroethyl)amino)-2- (2,5- difluorophenyl)piperidine- 1-carbonyl)-7- azaspiro[4.5]decan-10- yl)methyl)pyrimidin- 4(3H)-one | 8.28 (s, 1H), 7.13 (td, 1H), 7.05-7.00 (m, 1H), 7.00-6.94 (m, 1H), 6.31 (s, 1H), 5.91 (tt, 1H), 4.23 (d, 1H), 4.01 (dd, 1H), 3.83 (dt, 1H), 3.60 (t, 1H), 3.37 (d, 1H), 3.30- 3.22 (m, 2H), 2.89 (td, 2H), 2.71 (d, 1H), 2.66-2.55 (m, 2H), 2.02 (br s, 1H), 1.96- 1.89 (m, 2H), 1.86 (qu, 1H), 1.82-1.69 (m, 2H), 1.68-1.52 (m, 3H), 1.52-1.34 (m, 2H), 1.34-1.08 (m, 6H), 0.94-0.87 (m, 4H). | (Method B) R_{T} = 0.90 min, m/z 590 [M + H]⁺ |
| 183 | 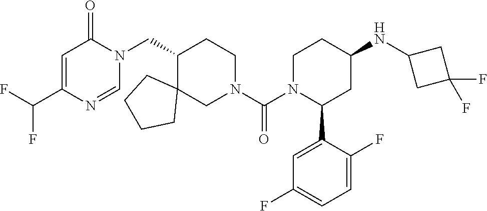 [SMILES: O=C(N1CC[C@@H](Cn2cnc(C(F)F)cc2=O)C2(CCCC2)C1)N1CC[C@@H](NC2CC(F)(F)C2)C[C@H]1c1cc(F)ccc1F] | 3-(((R)-7-((2S,4R)-4- ((3,3- Difluorocyclobutyl)amino)- 2-(2,5- difluorophenyl)piperidine- 1-carbonyl)-7- azaspiro[4.5]decan-10- yl)methyl)-6- (difluoromethyl)pyrimidin- 4(3H)-one | 8.58 (s, 1H), 7.13 (td, 1H), 7.05-6.99 (m, 1H), 6.99-6.94 (m, 1H), 6.75 (t, 1H), 6.66 (s, 1H), 4.23 (d, 1H), 4.08 (dd, 1H), 3.83 (td, 1H), 3.72 (t, 1H), 3.45-3.34 (m, 1H), 3.30-3.22 (m, 2H), 3.22-3.14 (m, 1H), 2.92-2.55 (m, 6H), 2.34-2.18 (m, 2H), 1.92-1.79 (m, 3H), 1.78-1.68 (m, 1H), 1.69-1.52 (m, 3H), 1.52-1.44 (m, 1H), 1.44-1.29 (m, 2H), 1.29-1.21 (m, 3H), 1.21-1.10 (m, 2H). | (Method B) R_{T} = 0.92 min, m/z 626 [M + H]⁺ |
| 184 | 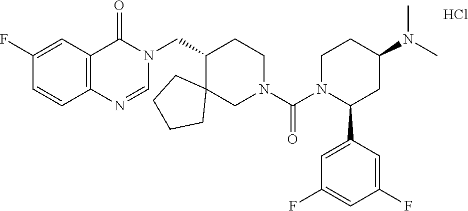 [SMILES: CN(C)[C@@H]1CCN(C(=O)N2CC[C@@H](Cn3cnc4ccc(F)cc4c3=O)C3(CCCC3)C2)[C@H](c2cc(F)cc(F)c2)C1 ; Cl] | 3-(((R)-7-((2S,4R)-2-(3,5- Difluorophenyl)-4- (dimethylamino)piperidine- 1-carbonyl)-7- azaspiro[4.5]decan-10- yl)methyl)-6- fluoroquinazolin-4(3H)- one hydrochloride | 10.23 (br s, 1H), 8.36 (s, 1H), 7.82 (dd, 1H), 7.79-7.70 (m, 2H), 7.06 (tt, 1H), 7.00-6.92 (m, 2H), 4.14 (dd, 1H), 4.05 (dd, 1H), 3.90-3.76 (m, 2H), 3.45-3.34 (m, 3H), 3.04-2.84 (m, 1H), 2.79 (d, 1H), 2.75-2.70 (m, 6H), 2.68 (t, 1H), 2.16-2.04 (m, 2H), 1.93-1.81 (m, 2H), 1.81-1.55 (m, 5H), 1.55-1.44 (m, 1H), 1.43-1.30 (m, 2H), 1.29-1.14 (m, 3H). | (Method B) R_{T} = 0.98 min, m/z 582 [M + H]⁺ |
| 185 | 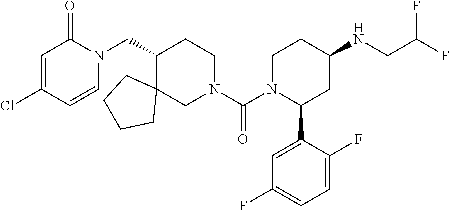 [SMILES: O=C(N1CC[C@@H](Cn2ccc(Cl)cc2=O)C2(CCCC2)C1)N1CC[C@@H](NCC(F)F)C[C@H]1c1cc(F)ccc1F] | 4-Chloro-1-(((R)-7- ((2S,4R)-4-((2,2- difluoroethyl)amino)-2- (2,5- difluorophenyl)piperidine- 1-carbonyl)-7- azaspiro[4.5]decan-10- yl)methyl)pyridin-2(1H)- one | 7.74 (d, J = 7.3 Hz, 1H), 7.14 (td, J = 9.3, 4.5 Hz, 1H), 7.05-6.94 (m, 2H), 6.52 (d, J = 2.4 Hz, 1H), 6.35 (dd, J = 7.3, 2.4 Hz, 1H), 5.91 (tt, J = 56.6, 4.2 Hz, 1H), 4.28- 4.19 (m, 1H), 4.04 (dd, J = 12.8, 3.1 Hz, 1H), 3.88-3.79 (m, 1H), 3.68 (s, 1H), 3.35 (d, J = 9.8 Hz, 1H), 3.29-3.23 (m, 1H, obscured by HDO signal), 2.89 (td, J = 16.1, 4.3 Hz, 3H), 2.74 (d, J = 12.9 Hz, 1H), 2.67-2.58 (m, 2H), 1.92 (dd, J = 12.9, 3.9 Hz, 3H), 1.83-1.68 (m, 2H), 1.69-1.08 (m, 11H). | (Method B) R_{T} = 0.87 min, m/z 583, 585 [M + H]⁺ |
| 186 | 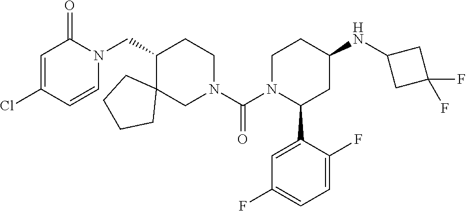 [SMILES: O=C(N1CC[C@@H](Cn2ccc(Cl)cc2=O)C2(CCCC2)C1)N1CC[C@@H](NC2CC(F)(F)C2)C[C@H]1c1cc(F)ccc1F] | 4-Chloro-1-(((R)-7- ((2S,4R)-4-((3,3- difluorocyclobutyl)amino)- 2-(2,5- difluorophenyl)piperidine- 1-carbonyl)-7- azaspiro[4.5]decan-10- yl)methyl)pyridin-2(1H)- one | 7.74 (d, J = 7.3 Hz, 1H), 7.13 (td, J = 9.3, 4.5 Hz, 1H), 7.06-6.93 (m, 2H), 6.52 (d, J = 2.4 Hz, 1H), 6.35 (dd, J = 7.2, 2.4 Hz, 1H), 4.27-4.19 (m, 1H), 4.04 (dd, J = 13.1, 3.2 Hz, 1H), 3.82 (d, J = 13.2 Hz, 1H), 3.69 (s, 1H), 3.40-3.32 (m, 1H), 3.29-3.13 (m, 2H), 2.86 (s, 1H), 2.74 (d, J = 12.9 Hz, 3H), 2.67-2.55 (m, 2H), 2.26 (s, 2H), 1.86 (d, J = 11.4 Hz, 2H), 1.83-1.68 (m, 2H), 1.68-1.06 (m, 12H). | (Method B) R_{T} = 0.90 min, m/z 609, 611 [M + H]⁺ |
| 187 | 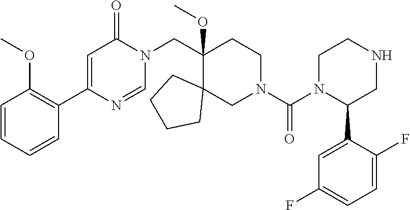 [SMILES: COc1ccccc1-c1cc(=O)n(C[C@]2(OC)CCN(C(=O)N3CCNC[C@H]3c3cc(F)ccc3F)CC23CCCC3)cn1] | 3-(((S)-7-((R)-2-(2,5- Difluorophenyl)piperazine- 1-carbonyl)-10- methoxy-7- azaspiro[4.5]decan-10- yl)methyl)-6-(2- methoxyphenyl)pyrimidin- 4(3H)-one | 8.31 (s, 1H), 8.02 (dd, J = 7.8, 1.8 Hz, 1H), 7.47 (ddd, J = 8.8, 7.3, 1.8 Hz, 1H), 7.17 (ddt, J = 14.0, 9.3, 5.2 Hz, 3H), 7.10- 7.03 (m, 3H), 4.47 (d, J = 14.2 Hz, 1H), 4.36 (dd, J = 9.1, 3.5 Hz, 1H), 3.98 (d, J = 14.2 Hz, 1H), 3.89 (s, 3H), 3.65 (s, 2H), 3.35 (d, J = 13.5 Hz, 1H), 3.16 (s, 4H), 2.97-2.71 (m, 5H), 2.68-2.61 (m, 1H, obscured by DMSO satellite), 1.67 (ddd, J = 35.3, 11.8, 6.2 Hz, 2H), 1.56 (dt, J = 16.3, 6.2 Hz, 3H), 1.43 (dq, J = 19.1, 9.1 Hz, 3H), 1.19 (d, J = 13.0 Hz, 1H), 1.05- 0.97 (m, 1H). [NH signal not observed]. | (Method B) R_{T} = 1.01 min, m/z 608 [M + H]⁺ |
| 188 | 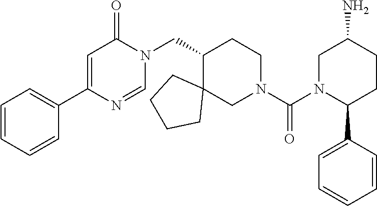 [SMILES: N[C@@H]1CC[C@@H](c2ccccc2)N(C(=O)N2CC[C@@H](Cn3cnc(-c4ccccc4)cc3=O)C3(CCCC3)C2)C1] | 3-(((R)-7-((2S,5R)-5- Amino-2- phenylpiperidine-1- carbonyl)-7- azaspiro[4.5]decan-10- yl)methyl)-6- phenylpyrimidin-4(3H)- one and 3-(((R)-7-((2R,5S)-5- amino-2- phenylpiperidine-1- | 8.58 (s, 0.5H), 8.55 (s, 0.5H), 8.09-8.03 (m, 2H), 7.53-7.45 (m, 3H), 7.32-7.12 (m, 5H), 6.97 (s, 0.5H), 6.97 (s, 0.5H), 4.14- 4.05 (m, 1.5H), 4.01 (dd, 0.5H), 3.90 (d, 0.5H), 3.81 (dt, 0.5H), 3.73 (t, 1H), 3.46 (d, 0.5H), 3.40 (d, 0.5H), 3.18-3.08 (m, 1H), 2.96 (t, 0.5H), 2.90-2.77 (m, 1.5H), 2.65 (d, 0.5H), 2.60-2.56 (m, 0.5H), 1.94- 1.81 (m, 2H), 1.81-1.71 (m, 2H), 1.69- 1.54 (m, 3H), 1.54-1.43 (m, 2H), 1.43- 1.15 (m, 6.5H), 1.15-1.07 (m, 0.5H). [NH₂ not observed]. | (Method B) R_{T} = 0.97 min, m/z 526 [M + H]⁺ |
| | | carbonyl)-7- | | |
| | | azaspiro[4.5]decan-10- | | |
| | | yl)methyl)-6- | | |
| | | phenylpyrimidin-4(3H)- | | |
| | | one | | |
| | and | | | |
| | 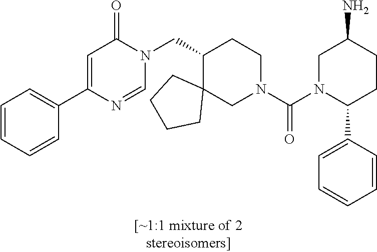 [SMILES: N[C@H]1CC[C@H](c2ccccc2)N(C(=O)N2CC[C@@H](Cn3cnc(-c4ccccc4)cc3=O)C3(CCCC3)C2)C1] | | | |
| 189 | 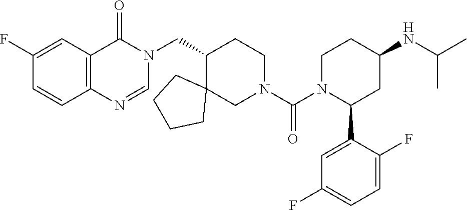 [SMILES: CC(C)N[C@@H]1CCN(C(=O)N2CC[C@@H](Cn3cnc4ccc(F)cc4c3=O)C3(CCCC3)C2)[C@H](c2cc(F)ccc2F)C1] | 3-(((R)-7-((2S,4R)-2-(2,5- Difluorophenyl)-4- (isopropylamino)piperidine- 1-carbonyl)-7- azaspiro[4.5]decan-10- yl)methyl)-6- fluoroquinazolin-4(3H)- one | 8.37 (s, 1H), 7.82 (dd, J = 8.6, 2.9 Hz, 1H), 7.79-7.69 (m, 2H), 7.13 (td, J = 9.3, 4.5 Hz, 1H), 6.99 (dddd, J = 25.5, 9.0, 6.5, 3.4 Hz, 2H), 4.25 (d, J = 11.6 Hz, 1H), 4.16 (dd, J = 13.3, 3.0 Hz, 1H), 3.88- 3.72 (m, 2H), 3.41 (d, J = 12.4 Hz, 1H), 3.29-3.23 (m, 1H), 2.84 (s, 2H), 2.74 (d, J = 12.9 Hz, 1H), 2.70-2.64 (m, 2H), 1.94-1.85 (m, 3H), 1.81 (s, 1H), 1.73- 1.45 (m, 4H), 1.44-1.14 (m, 8H), 0.93 (dd, J = 7.6, 6.2 Hz, 6H). | (Method B) R_{T} = 0.93 min, m/z 596 [M + H]⁺ |
| 190 | 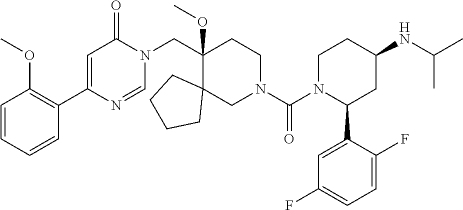 [SMILES: COc1ccccc1-c1cc(=O)n(C[C@]2(OC)CCN(C(=O)N3CC[C@@H](NC(C)C)C[C@H]3c3cc(F)ccc3F)CC23CCCC3)cn1] | 3-(((S)-7-((2S,4R)-2-(2,5- Difluorophenyl)-4- (isopropylamino)piperidine- 1-carbonyl)-10- methoxy-7- azaspiro[4.5]decan-10- yl)methyl)-6-(2- methoxyphenyl)pyrimidin- 4(3H)-one | 8.31 (s, 1H), 8.03 (dd, J = 7.8, 1.8 Hz, 1H), 7.47 (ddd, J = 8.7, 7.3, 1.8 Hz, 1H), 7.20-7.11 (m, 2H), 7.11-6.98 (m, 4H), 4.49 (d, J = 14.1 Hz, 1H), 4.24 (d, J = 11.4 Hz, 1H), 3.97 (d, J = 14.2 Hz, 1H), 3.90 (s, 3H), 3.72-3.62 (m, 1H), 3.40 (d, J = 12.8 Hz, 1H), 3.29-3.21 (m, 1H), 3.15 (s, 3H), 2.99-2.80 (m, 2H), 2.73-2.62 (m, 2H, obscured by DMSO satellite), 2.46-2.38 (m, 1H, obscured by DMSO signal), 1.90 (d, J = 12.4 Hz, 2H), 1.75-1.67 (m, 1H), 1.67-1.59 (m, 1H), 1.59-1.30 (m, 7H), 1.30-1.12 (m, 3H), 0.98-0.88 (m, 7H). | (Method B) R_{T} = 1.02 min, m/z 664 [M + H]⁺ |
| 191 | 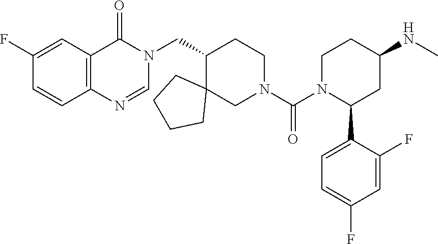 [SMILES: CN[C@@H]1CCN(C(=O)N2CC[C@@H](Cn3cnc4ccc(F)cc4c3=O)C3(CCCC3)C2)[C@H](c2ccc(F)cc2F)C1] | 3-(((R)-7-((2S,4R)-2-(2,4- Difluorophenyl)-4- (methylamino)piperidine- 1-carbonyl)-7- azaspiro[4.5]decan-10- yl)methyl)-6- fluoroquinazolin-4(3H)- one | 8.37 (s, 1H), 7.82 (dd, J = 8.7, 2.9 Hz, 1H), 7.79-7.70 (m, 2H), 7.25 (td, J = 8.4, 6.6 Hz, 1H), 7.08 (ddd, J = 10.7, 9.4, 2.6 Hz, 1H), 6.94 (td, J = 8.5, 2.6 Hz, 1H), 4.23 (dd, J = 12.0, 3.0 Hz, 1H), 4.15 (dd, J = 13.3, 3.0 Hz, 1H), 3.87-3.72 (m, 2H), 3.39 (d, J = 12.9 Hz, 1H), 3.29- 3.24 (m, 1H), 2.82 (s, 1H), 2.71-2.64 (m, 2H), 2.47-2.38 (m, 1H), 2.24 (s, 3H), 1.96-1.83 (m, 3H), 1.78 (s, 1H), 1.72- 1.43 (m, 5H), 1.41-1.28 (m, 3H), 1.28- 1.13 (m, 4H). | (Method B) R_{T} = 0.90 min, m/z 568 [M + H]⁺ |
| 192 | 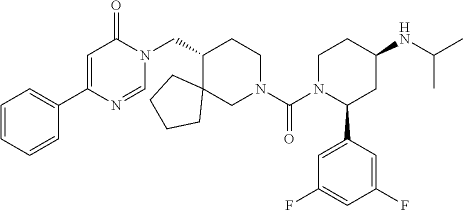 [SMILES: CC(C)N[C@@H]1CCN(C(=O)N2CC[C@@H](Cn3cnc(-c4ccccc4)cc3=O)C3(CCCC3)C2)[C@H](c2cc(F)cc(F)c2)C1] | 3-(((R)-7-((2S,4R)-2-(3,5- Difluorophenyl)-4- (isopropylamino)piperidine- 1-carbonyl)-7- azaspiro[4.5]decan-10- yl)methyl)-6- phenylpyrimidin-4(3H)- one | 8.55 (s, 1H), 8.10-8.01 (m, 2H), 7.54-7.45 (m, 3H), 7.02-6.93 (m, 2H), 6.92-6.84 (m, 2H), 4.11 (dd 1H), 4.01 (dd, 1H), 3.85 (dt, 1H), 3.72 (t, 1H), 3.41 (d, 1H), 3.28-3.21 (m, 1H), 2.92-2.78 (m, 2H), 2.74 (d, 1H), 2.69-2.60 (m, 2H), 1.95-1.83 (m, 3H), 1.83-1.74 (m, 1H), 1.72-1.61 (m, 2H), 1.61-1.44 (m, 2H), 1.42-1.11 (m, 8H), 0.98-0.88 (m, 6H). | (Method K) R_{T} = 0.93 min, m/z 604 [M + H]⁺ |
| 193 | 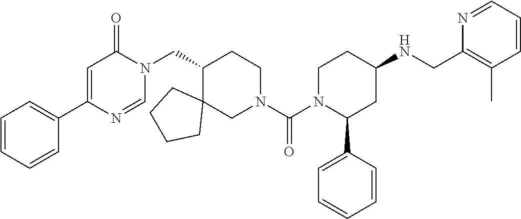 [SMILES: Cc1cccnc1CN[C@@H]1CCN(C(=O)N2CC[C@@H](Cn3cnc(-c4ccccc4)cc3=O)C3(CCCC3)C2)[C@H](c2ccccc2)C1] | 3-(((R)-7-((2S,4R)-4-(((3- Methylpyridin-2- yl)methyl)amino)-2- phenylpiperidine-1- carbonyl)-7- azaspiro[4.5]decan-10- yl)methyl)-6- phenylpyrimidin-4(3H)- one | 8.55 (s, 1H), 8.30 (dd, J = 4.9, 1.6 Hz, 1H), 8.09-8.03 (m, 2H), 7.50 (dtt, J = 8.4, 5.3, 2.5 Hz, 4H), 7.22 (d, J = 6.1 Hz, 4H), 7.18-7.10 (m, 2H), 6.97 (s, 1H), 4.10 (dd, J = 13.0, 3.1 Hz, 1H), 3.97 (dd, J = 11.7, 3.0 Hz, 1H), 3.88 (d, J = 13.2 Hz, 1H), 3.80 (t, J = 4.8 Hz, 2H), 3.70 (t, J = 12.0 Hz, 1H), 3.44 (d, J = 12.9 Hz, 1H), 3.29-3.22 (m, 1H), 2.84 (s, 1H), 2.73-2.61 (m, 3H), 2.27 (s, 3H), 2.20 (s, 1H), 1.99 (dd, J = 12.7, 4.0 Hz, 2H), 1.85 (ddt, J = 10.5, 7.0, 3.4 Hz, 1H), 1.77 (s, 1H), 1.72-1.40 (m, 5H), 1.39-1.24 (m, 4H), 1.19 (td, J = 15.9, 14.2, 8.6 Hz, 2H). | (Method K) R_{T} = 0.95 min, m/z 631 [M + H]⁺ |
| 194 | 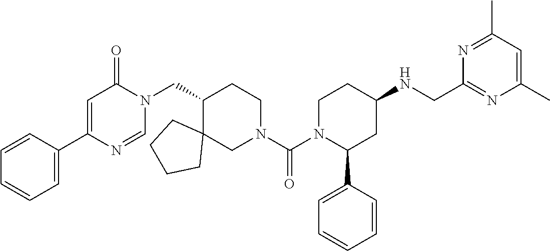 [SMILES: Cc1cc(C)nc(CN[C@@H]2CCN(C(=O)N3CC[C@@H](Cn4cnc(-c5ccccc5)cc4=O)C4(CCCC4)C3)[C@H](c3ccccc3)C2)n1] | 3-(((R)-7-((2S,4R)-4- (((4,6-Dimethylpyrimidin- 2-yl)methyl)amino)-2- phenylpiperidine-1- carbonyl)-7- azaspiro[4.5]decan-10- yl)methyl)-6- phenylpyrimidin-4(3H)- one | 8.55 (s, 1H), 8.06 (dd, J = 6.7, 3.0 Hz, 2H), 7.49 (dd, J = 5.0, 1.9 Hz, 3H), 7.26- 7.18 (m, 4H), 7.13 (d, J = 6.4 Hz, 1H), 7.10 (s, 1H), 6.97 (s, 1H), 4.10 (dd, J = 13.1, 3.0 Hz, 1H), 3.96 (dd, J = 11.6, 3.1 Hz, 1H), 3.87 (dd, J = 10.8, 6.7 Hz, 1H), 3.80 (s, 2H), 3.70 (t, J = 12.0 Hz, 1H), 3.44 (d, J = 12.9 Hz, 1H), 3.29-3.22 (m, 2H, obscured by HDO signal), 2.84 (s, 1H), 2.72-2.62 (m, 3H), 2.38 (s, 6H), 2.02-1.92 (m, 2H), 1.90-1.81 (m, 1H), 1.76 (s, 1H), 1.71-1.39 (m, 5H), 1.39- 1.13 (m, 6H). | (Method K) R_{T} = 0.91 min, m/z 646 [M + H]⁺ |
| 195 | 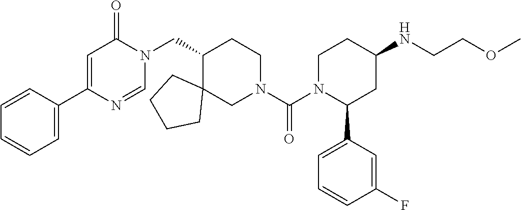 [SMILES: COCCN[C@@H]1CCN(C(=O)N2CC[C@@H](Cn3cnc(-c4ccccc4)cc3=O)C3(CCCC3)C2)[C@H](c2cccc(F)c2)C1] | 3-(((R)-7-((2S,4R)-2-(3- Fluorophenyl)-4-((2- methoxyethyl)amino) piperidine-1-carbonyl)-7- azaspiro[4.5]decan-10- yl)methyl)-6- phenylpyrimidin-4(3H)- one | 8.55 (s, 1H), 8.10-8.02 (m, 2H), 7.53-7.44 (m, 3H), 7.25 (dt, 1H), 7.04 (d, 1H), 7.00- 6.93 (m, 3H), 4.10 (dd, 1H), 3.99 (dd, 1H), 3.86 (dt, 1H), 3.71 (t, 1H), 3.42 (d, 1H), 3.34-3.32 (m, 2H), 3.25 (dt, 1H), 3.21 (s, 3H), 2.84 (br s, 1H), 2.73-2.61 (m, 4H), 2.60-2.54 (m, 1H), 1.96-1.82 (m, 3H), 1.82-1.72 (m, 1H), 1.71-1.43 (m, 5H), 1.41-1.15 (m, 7H). | (Method K) R_{T} = 0.89 min, m/z 602 [M + H]⁺ |
| 196 | 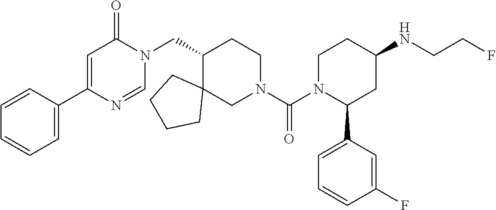 [SMILES: O=C(N1CC[C@@H](Cn2cnc(-c3ccccc3)cc2=O)C2(CCCC2)C1)N1CC[C@@H](NCCF)C[C@H]1c1cccc(F)c1] | 3-(((R)-7-((2S,4R)-4-((2- Fluoroethyl)amino)-2-(3- fluorophenyl)piperidine- 1-carbonyl)-7- azaspiro[4.5]decan-10- yl)methyl)-6- phenylpyrimidin-4(3H)- one | 8.55 (s, 1H), 8.10-8.02 (m, 2H), 7.53-7.45 (m, 3H), 7.26 (dt, 1H), 7.04 (d, 1H), 7.01- 6.93 (m, 3H), 4.45 (t, 1H), 4.35 (t, 1H), 4.10 (dd, 1H), 3.99 (dd, 1H), 3.86 (dt, 1H), 3.71 (t, 1H), 3.43 (d, 1H), 3.27 (dt, 1H), 2.91-2.79 (m, 2H), 2.77 (t, 1H), 2.73-2.54 (m, 3H), 2.00-1.70 (m, 5H), 1.70-1.43 (m, 4H), 1.42-1.14 (m, 7H). | (Method K) R_{T} = 0.87 min, m/z 590 [M + H]⁺ |
| 197 | 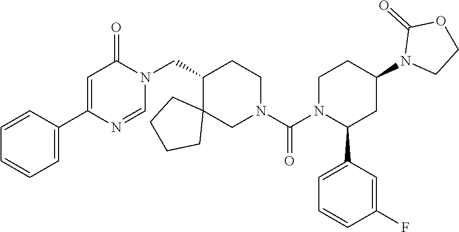 [SMILES: O=C1OCCN1[C@@H]1CCN(C(=O)N2CC[C@@H](Cn3cnc(-c4ccccc4)cc3=O)C3(CCCC3)C2)[C@H](c2cccc(F)c2)C1] | 3-((2S,4R)-2-(3- Fluorophenyl)-1-((R)-10- ((6-oxo-4- phenylpyrimidin-1(6H)- yl)methyl)-7- azaspiro[4.5]decane-7- carbonyl)piperidin-4- yl)oxazolidin-2-one | 8.55 (s, 1H), 8.08-8.03 (m, 2H), 7.52-7.46 (m, 3H), 7.27 (dt, 1H), 7.08 (d, 1H), 7.03 (d, 1H), 7.00-6.95 (m, 2H), 4.27-4.16 (m, 2H), 4.13-4.04 (m, 2H), 3.89 (dt, 1H), 3.78-3.66 (m, 2H), 3.51 (t, 2H), 3.41 (d, 1H), 3.34-3.32 (m, 1H), 2.93 (br s, 1H), 2.78-2.69 (m, 2H), 1.92-1.82 (m, 2H), 1.81-1.71 (m, 3H), 1.71-1.53 (m, 4H), 1.52-1.44 (m, 1H), 1.41-1.31 (m, 2H), 1.30-1.14 (m, 3H). | (Method K) R_{T} = 1.36 min, m/z 614 [M + H]⁺ |
| 198 | 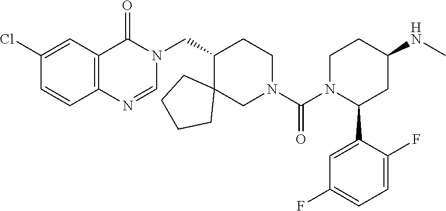 [SMILES: CN[C@@H]1CCN(C(=O)N2CC[C@@H](Cn3cnc4ccc(Cl)cc4c3=O)C3(CCCC3)C2)[C@H](c2cc(F)ccc2F)C1] | 6-Chloro-3-(((R)-7- ((2S,4R)-2-(2,5- difluorophenyl)-4- (methylamino)piperidine- 1-carbonyl)-7- azaspiro[4.5]decan-10- yl)methyl)quinazolin- 4(3H)-one | 8.40 (s, 1H), 8.09 (d, 1H), 7.86 (dd, 1H), 7.72 (d, 1H), 7.13 (td, 1H), 7.06-6.94 (m, 2H), 4.26 (d, 1H), 4.15 (dd, 1H), 3.88-3.74 (m, 2H), 3.40 (d, 1H), 3.29-3.24 (m, 1H), 2.83 (br s, 1H), 2.74 (d, 1H), 2.66 (t, 1H), 2.47-2.39 (m, 1H), 2.25 (s, 3H), 1.96-1.84 (m, 3H), 1.84-1.73 (m, 1H), 1.72-1.45 (m, 5H), 1.43-1.14 (m, 7H). | (Method K) R_{T} = 0.87 min, m/z 584 [M + H]⁺ |
| 199 | 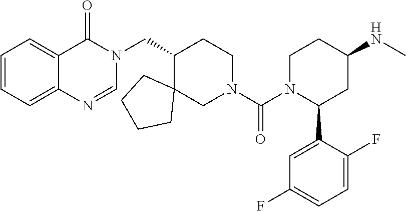 [SMILES: CN[C@@H]1CCN(C(=O)N2CC[C@@H](Cn3cnc4ccccc4c3=O)C3(CCCC3)C2)[C@H](c2cc(F)ccc2F)C1] | 3-(((R)-7-((2S,4R)-2-(2,5- Difluorophenyl)-4- (methylamino)piperidine- 1-carbonyl)-7- azaspiro[4.5]decan-10- yl)methyl)quinazolin- 4(3H)-one | 8.37 (s, 1H), 8.16 (dd, J = 7.9, 1.5 Hz, 1H), 7.83 (ddd, J = 8.5, 7.1, 1.6 Hz, 1H), 7.68 (dd, J = 8.2, 1.1 Hz, 1H), 7.56 (ddd, J = 8.2, 7.1, 1.2 Hz, 1H), 7.14 (td, J = 9.3, 4.5 Hz, 1H), 7.05-6.94 (m, 2H), 4.26 (d, J = 11.7, 2.6 Hz, 1H), 4.16 (dd, J = 13.4, 3.0 Hz, 1H), 3.85 (dt, J = 13.4, 4.5 Hz, 1H), 3.78 (t, J = 12.2 Hz, 1H), 3.40 (d, J = 12.9 Hz, 1H), 3.33-3.25 (m, 2H, obscured by HDO signal), 2.84 (s, 1H), 2.74 (d, J = 12.9 Hz, 1H), 2.69-2.63 (m, 1H), 2.48-2.40 (m, 1H), 2.25 (s, 3H), 1.91 (ddd, J = 17.8, | (Method K) R_{T} = 0.80 min, m/z 550 [M + H]⁺ |
| | | | 13.6, 7.0 Hz, 3H), 1.81 (s, 1H), 1.71- | |
| | | | 1.44 (m, 4H), 1.44-1.13 (m, 7H). | |
| 200 | 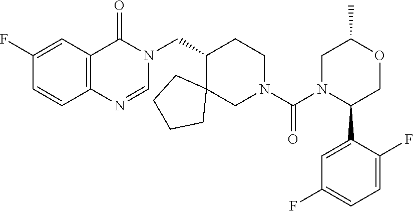 [SMILES: C[C@H]1CN(C(=O)N2CC[C@@H](Cn3cnc4ccc(F)cc4c3=O)C3(CCCC3)C2)[C@H](c2cc(F)ccc2F)CO1] | 3-(((R)-7-((2S,5R)-5-(2,5- Difluorophenyl)-2- methylmorpholine-4- carbonyl)-7- azaspiro[4.5]decan-10- yl)methyl)-6- fluoroquinazolin-4(3H)- one | 8.54 (s, 1H), 8.06 (dd, J = 6.7, 3.0 Hz, 2H), 7.49 (dd, J = 5.1, 1.9 Hz, 3H), 7.19 (td, J = 9.2, 4.5 Hz, 1H), 7.13-7.02 (m, 2H), 6.97 (s, 1H), 4.31 (ddd, J = 10.9, 3.9, 1.4 Hz, 1H), 4.11 (dd, J = 13.0, 3.1 Hz, 1H), 3.89 (dd, J = 13.4, 4.3 Hz, 1H), 3.81 (dtd, J = 12.4, 6.2, 2.2 Hz, 1H), 3.72 (dd, J = 11.4, 3.7 Hz, 2H), 3.41 (t, J = 11.2 Hz, 2H), 3.21 (dd, J = 12.0, 2.3 Hz, 1H), 2.78 (d, J = 12.9 Hz, 2H), 2.44 (t, J = 11.2 Hz, 1H), 1.88 (tt, J = 10.5, 3.8 Hz, 1H), 1.79 (s, 1H), 1.67 (s, 3H), 1.49 (dt, J = 12.2, 8.2 Hz, 1H), 1.34 (dd, J = | (Method A) R_{T} = 1.54 min, m/z 555 [M + H]⁺ |
| | | | 21.2, 11.7 Hz, 2H), 1.23 (p, J = 7.9 Hz, | |
| | | | 3H), 1.15 (d, J = 6.2 Hz, 3H). | |
| 201 | 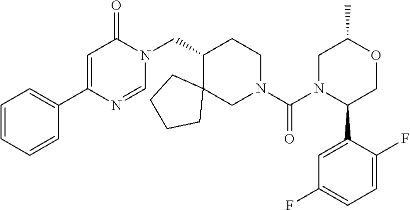 [SMILES: C[C@H]1CN(C(=O)N2CC[C@@H](Cn3cnc(-c4ccccc4)cc3=O)C3(CCCC3)C2)[C@H](c2cc(F)ccc2F)CO1] | 3-(((R)-7-((2,5R)-5-(2,5- Difluorophenyl)-2- methylmorpholine-4- carbonyl)-7- azaspiro[4.5]decan-10- yl)methyl)-6- phenylpyrimidin-4(3H)- one | 8.54 (s, 1H), 8.06 (dd, J = 6.7, 3.0 Hz, 2H), 7.49 (dd, J = 5.1, 1.9 Hz, 3H), 7.19 (td, J = 9.2, 4.5 Hz, 1H), 7.13-7.02 (m, 2H), 6.97 (s, 1H), 4.31 (ddd, J = 10.9, 3.9, 1.4 Hz, 1H), 4.11 (dd, J = 13.0, 3.1 Hz, 1H), 3.89 (dd, J = 13.4, 4.3 Hz, 1H), 3.81 (dtd, J = 12.4, 6.2, 2.2 Hz, 1H), 3.72 (dd, J = 11.4, 3.7 Hz, 2H), 3.41 (t, J = 11.2 Hz, 2H), 3.21 (dd, J = 12.0, 2.3 Hz, 1H), 2.78 (d, J = 12.9 Hz, 2H), 2.44 (t, J = 11.2 Hz, 1H), 1.88 (tt, J = 10.5, 3.8 Hz, 1H), 1.79 (s, 1H), 1.67 (s, 3H), 1.49 (dt, J = 12.2, 8.2 Hz, 1H), 1.34 (dd, J = | (Method K) R_{T} = 1.62 min, m/z 563 [M + H]⁺ |
| | | | 21.2, 11.7 Hz, 2H), 1.23 (p, J = 7.9 Hz, | |
| | | | 3H), 1.15 (d, J = 6.2 Hz, 3H). | |

USP19 inhibitor compounds are also disclosed in WO2018/020242, WO2020/115500, WO2019/150119, and WO2020/115501, each of which is expressly incorporated herein by reference. Compounds according to formula (I) that are analogs of the compounds disclosed in WO2018/020242, WO2020/115500, WO2019/150119, and WO2020/115501—i.e. analogs where the R0 position is H, F, NH2, or OCH3—are expressly incorporated herein and can be obtained by the skilled person following the synthesis protocols provided herein and in WO2018/020242, WO2020/115500, WO2019/150119, and WO2020/115501.

1. A compound of formula (I): 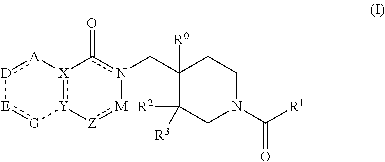
wherein
R⁰ is H, NH2, F, or OCH3;
R¹ is NR^{a}R^{b} or NR^{a}CH2R^{b}, wherein R^{a} and R^{b} are independently selected from H, methyl, ethyl, propyl, CF3, optionally substituted cyclopropyl, optionally substituted cyclobutyl, optionally substituted cycopentyl, optionally substituted cyclohexyl, optionally substituted phenyl, optionally substituted benzyl, optionally substituted pyridinyl, pyrazole, imidazole, furan, benzodioxol, optionally substituted oxadiazole, thiazole, and thiophene, wherein the optional substituents are independently selected from halo, methyl, cyclopropyl and CN,
or wherein R¹ is NR^{a}R^{b} and wherein R^{a} and R^{b} together form an optionally substituted C3-C9 heterocycle together with the N to which they are attached,
wherein each of the one or more optional substituents is selected from OH, oxo, C1-C3 alkyl optionally substituted with OH and/or halo, optionally substituted phenyl, optionally substituted benzyl, C1-C3 alkoxy, NR^{m}Rⁿ, NHC(O)R^{m}, and NHCH2Rⁿ,
wherein R^{m} and Rⁿ are independently selected from H: C1-C3 alkyl optionally substituted with OH, methoxy or halo: C3-C4 cycloalkyl optionally substituted with methyl and/or halo: C3-C4 heterocycloalkyl optionally substituted with oxo, methyl or fluoro-methyl; C3-C5 heteroaryl optionally substituted with methyl; and Boc; and/or wherein Rⁿ is further selected from CH2OCH3, COOH and COOCH3,
or wherein R^{m} and Rⁿ form a C3-C5 heterocyclyl group together with the N to which they are attached, optionally wherein R^{m} and Rⁿ form a morpholinyl group together with the N to which they are attached;
R² and R³ are independently selected from H, and C1-6 alkyl, or together form a C3-C6 cycloalkyl or C3-C6 heterocycloalkyl with the carbon to which they attached;
X is absent, C, CR^{4a}, CR^{4a}R^{4b}, N, NR^{4a}, or C═O,
wherein R^{4a} and R^{4b} are independently selected from H, optionally substituted C1-C6 alkyl and halo,
wherein for each optionally substituted group, each one or more optional substituent is independently selected from C1-C4 alkyl, C3-C4 cycloalkyl, halo, CHF2, CF3 hydroxyl, NH2, NHCH3, NHCH2CH3, NO2, CH2OH, CH2OCH3, methoxy, OCHF2, OCF3, cyclopropyloxy, phenyl, fluoro-substituted phenyl (e.g. difluoro-substituted phenyl), benzyl, and oxo;
or wherein R^{4a} and R^{4b} together form a 3-06 cycloalkyl or C3-C6 heterocycloalkyl including the carbon to which they are attached;
Y is C, CR⁵, CR⁵R⁶, N, NR⁵, or O,
wherein R⁵ and R⁶ are independently selected from H, halo, optionally substituted C1-C6 alkyl, C2-C6 alkenyl, C2-C6 alkynyl, optionally substituted C3-C6 cycloalkyl, optionally substituted C3-C6 heterocycloalkyl, optionally substituted C5-C8 aryl, optionally substituted C6-C9 arylalkyl, optionally substituted C3-C8 heteroaryl, CH2OH, NR′R″, NS(O)R′R″, SO2R′, C(O)R′, COR′, C(O)OR′, C(O)NR′R″, OR′, wherein R′ and R″ are independently selected from H, C1-C6 alkyl, C5-C8 aryl, C6-C9 arylalkyl, and C3-C8 heteroaryl, or wherein R⁵ is NR′R″ and R′ and R″ together form an optionally substituted C3-6 heterocycloalkyl including the nitrogen to which they are attached, wherein for each optionally substituted group, each one or more optional substituent is independently selected from C1-C4 alkyl, C3-C4 cycloalkyl, halo, CHF2, CF3, hydroxyl, NH2, NHCH3, NHCH2CH3, NO2, CH2OH, CH2OCH3, methoxy, OCHF2, OCF3, cyclopropyloxy, phenyl, fluoro-substituted phenyl (e.g. difluoro-substituted phenyl), benzyl, and oxo,
or wherein R⁵ and R⁶ together form a C3-6 cycloalkyl or C3-C6 heterocycloalkyl including the carbon to which they are attached;
Z is N, NR⁷, C, CR⁷, CR⁷R⁸, or C═O,
wherein R⁷ and R⁸ are independently selected from H, halo, cyano, oxo, optionally substituted C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, C(O)OR^{c}, CONR^{c}R^{d}, NR^{c}R^{d}, NS(O)R^{c}R^{d}, S(O)(R^{c})NR^{d}, SOR^{c}, SO2R^{c}, and SR^{c}, wherein R^{c} and R^{d} are independently H, C1-C6 alkyl, C3-C6 cycloalkyl, C5-C6 aryl, C6-C9 arylalkyl, C3-C6 heteroaryl, CN, COOH, or COCH3 or R^{c} and R^{d} together form an optionally substituted C3-C7 heterocycle together with the heteroatom to which they are attached, wherein for each optionally substituted group, each one or more optional substituent is independently selected from C1-C4 alkyl, C3-C4 cycloalkyl, halo, CHF2, CF3, hydroxyl, NH2, NHCH3, NHCH2CH3, NO2, CH2OH, CH2OCH3, methoxy, OCHF2, OCF3, cyclopropyloxy, phenyl, fluoro-substituted phenyl (e.g. difluoro-substituted phenyl), benzyl, and oxo;
or wherein R⁷ and R⁸ together form a C3-6 cycloalkyl or C3-C6 heterocycloalkyl including the carbon to which they are attached;
M is absent, C, CR¹³ or CR¹³R¹⁴, wherein R¹³ and R¹⁴ are independently selected from H, and C1-C6 alkyl, or wherein R¹³ and R¹⁴ together form a C3-C6 cycloalkyl or C3-C6 heterocycloalkyl together with the carbon to which they are attached;
and
A is CR⁹, CHR⁹, N, NR⁹, S, or O;
D is CR⁹, CHR⁹, N or NR⁹,
G is absent, CR⁹, CHR⁹, or N,
wherein R⁹ is independently selected from H, halo, C1-C6 alkyl, CF3, and OR*, wherein R* is selected from optionally substituted C1-C6 alkyl, optionally substituted C3-C6 cycloalkyl, and optionally substituted C3-C6 heterocycloalkyl, wherein for each optionally substituted group, each one or more optional substituent is independently selected from C1-C4 alkyl, C3-C4 cycloalkyl, halo, CHF2, CF3, hydroxyl, NH2, NHCH3, NHCH2CH3, NO2, CH2OH, CH2OCH3, methoxy, OCHF2, OCF3, cyclopropyloxy, phenyl, fluoro-substituted phenyl (e.g. difluoro-substituted phenyl), benzyl, and oxo,
E is CR¹⁰, CHR¹⁰, N, NR¹⁰, S, or O,
wherein R¹⁰ is selected from H, halo, C1-C6 alkyl, C3-C6 cycloalkyl, C5-C8 aryl, C6-C9 arylalkyl, C4-C8 heteroaryl, SR^{x}, OR^{x}, NR^{x}R^{y}, and NS(O)R^{x}R^{y}, S(O)(R^{x})NR^{y}
wherein R^{x} and R^{y} are independently selected from H, C1-C6 alkyl, CF3, C3-C6 cycloalkyl, C5-C8 aryl, C6-C9 arylalkyl, C4-C8 heteroaryl, COOH, amido, cyano, C2-C6 alkene, C2-C6 alkyne, or wherein R^{x} and R^{y} together form an optionally substituted C4-C6 heterocycloalkyl together with the nitrogen to which they are attached, wherein for each optionally substituted group, each one or more optional substituent is independently selected from C1-C4 alkyl, C3-C4 cycloalkyl, halo, CHF2, CF3, hydroxyl, NH2, NHCH3, NHCH2CH3, NO2, CH2OH, CH2OCH3, methoxy, OCHF2, OCF3, cyclopropyloxy, phenyl, fluoro-substituted phenyl (e.g. difluoro-substituted phenyl), benzyl, and oxo;
or A, D, E and G are all absent, optionally wherein X and M are absent, or optionally wherein Y and Z together form an optionally substituted C5-C6 aryl or C3-C6 heteroaryl fused ring or Z and M together form an optionally substituted C5-C6 aryl or C5-C6 heteroaryl fused ring, wherein for each optionally substituted group, each one or more optional substituent is independently selected from C1-C4 alkyl, C3-C4 cycloalkyl, halo, CHF2, CF3, hydroxyl, NH2, NHCH3, NHCH2CH3, NO2, CH2OH, CH2OCH3, methoxy, OCHF2, OCF3, cyclopropyloxy, phenyl, fluoro-substituted phenyl (e.g. difluoro-substituted phenyl), benzyl, and oxo;
or a compound having a structure selected from the group consisting of: 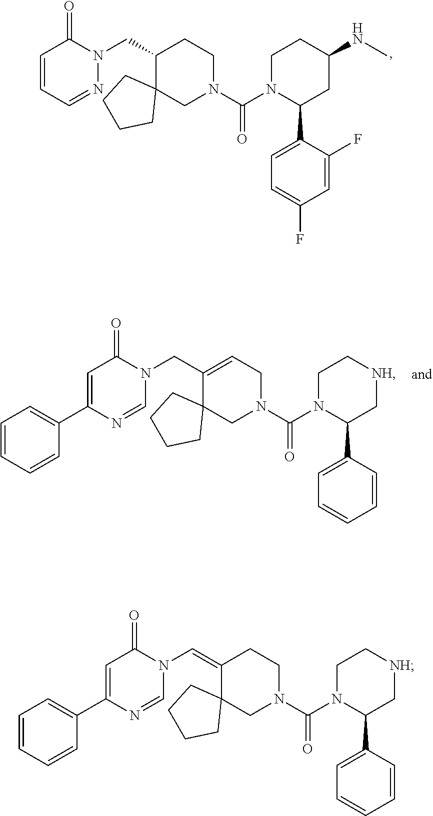 [SMILES: CN[C@@H]1CCN(C(=O)N2CC[C@@H](Cn3ncccc3=O)C3(CCCC3)C2)[C@H](c2ccc(F)cc2F)C1 ; O=C(N1CC=C(Cn2cnc(-c3ccccc3)cc2=O)C2(CCCC2)C1)N1CCNC[C@H]1c1ccccc1 ; O=C(N1CC/C(=C/n2cnc(-c3ccccc3)cc2=O)C2(CCCC2)C1)N1CCNC[C@H]1c1ccccc1]
or a stereoisomer, tautomer, hydrate, N-oxide derivative or pharmaceutically acceptable salt thereof.

2-9. (canceled)

10. The compound, stereoisomer, tautomer, hydrate, N-oxide derivative or pharmaceutically acceptable salt of claim 1, wherein R¹ is NR^{a}R^{b} and wherein R^{a} and R^{b} together form an optionally substituted C3-C9 heterocycle together with the N to which they are attached, optionally substituted with OH, CH2OH, CH2OCH3, oxo, NH2, C1-C3 aminoalkyl, amino-thietane dioxide, methyl, ethyl, propyl, CF3, phenyl, substituted phenyl, and benzyl;
wherein the heterocycle is optionally substituted with one or more substituents independently selected from methyl, NH2, C1-C2 aminoalkyl, CH2CF3, oxo, thiophene, and phenyl optionally substituted with one or more substituents independently selected from F and CF3, and OH provided the same ring carbon is not also substituted with methyl.

11-12. (canceled)

13. The compound, stereoisomer, tautomer, hydrate, N-oxide derivative or pharmaceutically acceptable salt of claim 1, wherein R^{a} and R^{b} together with the N to which they are attached form a heterocycle together with the N to which they are attached, wherein the heterocycle is selected from a piperidinyl or piperazinyl ring, wherein the heterocycle is optionally substituted with one or more substituents independently selected from methyl, NH2, NHCH3, NHCH2CH3, CH2CF3, oxo, thiophene, phenyl optionally substituted with F or CF3, and OH provided the same ring carbon is not also substituted with methyl, and;
wherein R¹ is selected from the group consisting of:

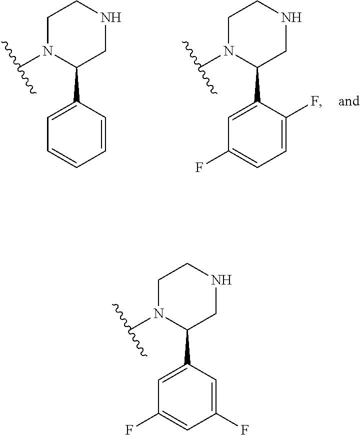 [SMILES: *N1CCNC[C@H]1c1ccccc1 ; *N1CCNC[C@H]1c1cc(F)ccc1F ; *N1CCNC[C@H]1c1cc(F)cc(F)c1]

14-15. (canceled)

16. The compound, stereoisomer, tautomer, hydrate, N-oxide derivative or pharmaceutically acceptable salt of claim 1, wherein R¹ is NR^{a}R^{b} and R^{a} and R^{b} together with the N to which they are attached form a piperidinyl group optionally substituted with phenyl, fluoro-phenyl, or difluoro-phenyl, and wherein the piperidinyl group is optionally further substituted with NR^{m}Rⁿ, NHC(O)R^{m}, or NHCH2Rⁿ,
wherein R^{m} and Rⁿ are independently selected from H; C1-C3 alkyl optionally substituted with OH, methoxy or halo; C3-C4 cycloalkyl optionally substituted with methyl and/or halo; C3-C4 heterocycloalkyl optionally substituted with oxo, methyl or fluoro-methyl; C3-C5 heteroaryl optionally substituted with methyl; and Boc; and/or wherein Rⁿ is further selected from CH2OCH3, COOH and COOCH3,
or wherein R^{m} and Rⁿ form a C3-C5 heterocyclyl group together with the N to which they are attached.

17. The compound, stereoisomer, tautomer, hydrate, N-oxide derivative or pharmaceutically acceptable salt of claim 1, wherein R¹ is NR^{a}R^{b} and R^{a} and R^{b} together with the N to which they are attached form a piperidinyl group, wherein the piperidinyl group is substituted at the 4 position with NR^{m}Rⁿ, NHC(O)R^{m}, and NHCH2Rⁿ, and is further substituted at the 2 position with phenyl, fluoro-phenyl, or difluoro-phenyl, and;
wherein R¹ is selected from:

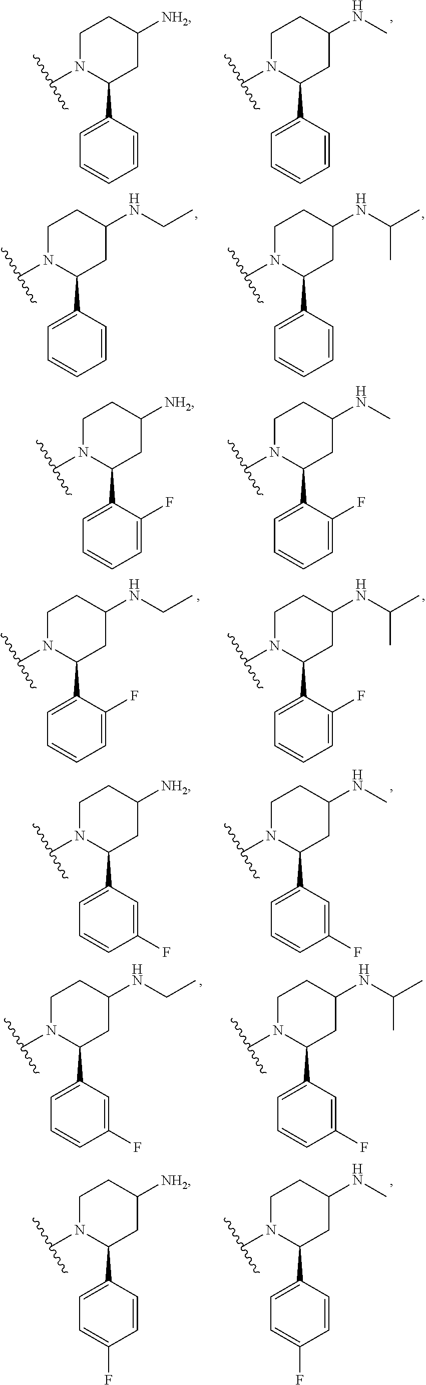

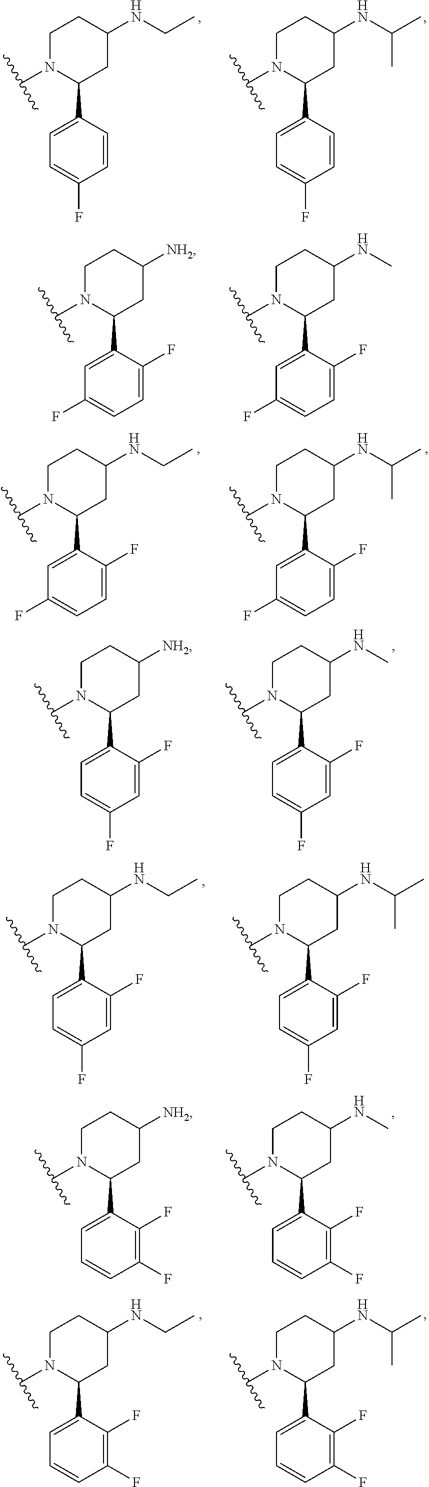

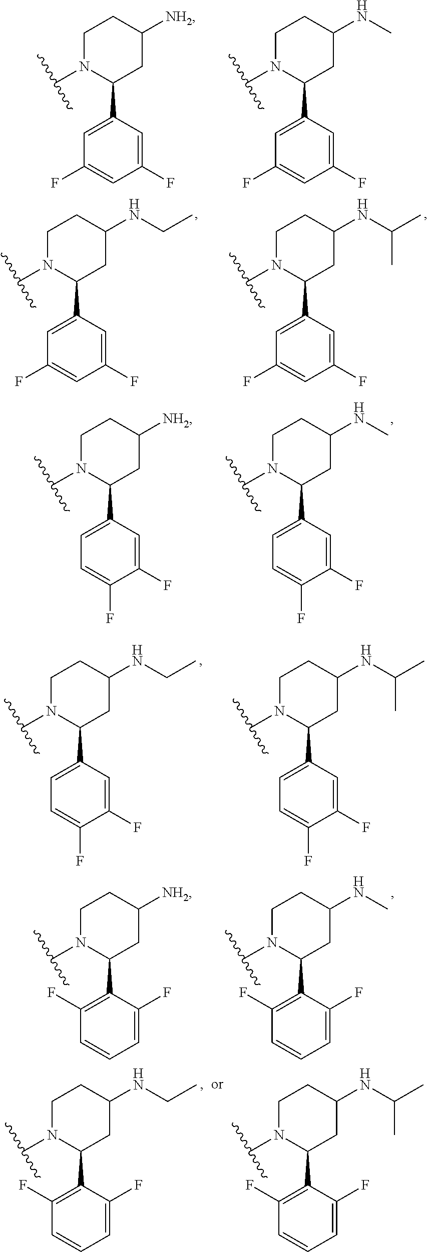

18-19. (canceled)

20. The compound, stereoisomer, tautomer, hydrate, N-oxide derivative or pharmaceutically acceptable salt of claim 1, wherein R¹ is NR^{a}CH2R^{b}, wherein R^{a} is H or methyl and R^{b} is selected from cyclobutyl optionally substituted with F, cyclohexyl, phenyl optionally substituted with F, furan and thiophene.

21. (canceled)

22. The compound, stereoisomer, tautomer, hydrate, N-oxide derivative or pharmaceutically acceptable salt of claim 1, wherein R² and R³ are independently selected from H, methyl and ethyl, or together form optionally substituted cyclopropyl, optionally substituted cyclobutyl, optionally substituted cyclopentyl, optionally substituted cyclohexyl, optionally substituted pyrrolidine, optionally substituted tetrahydropyran or optionally substituted tetrahydrofuran together with the carbon to which they are attached; wherein for each optionally substituted group, each one or more optional substituent is independently selected from C1-C4 alkyl, C3-C4 cycloalkyl, halo, CHF2, CF3, hydroxyl, NH2, NHCH3, NHCH2CH3, NO2, CH2OH, CH2OCH3, methoxy, OCHF2, OCF3, cyclopropyloxy, phenyl, fluoro-substituted phenyl (e.g. difluoro-substituted phenyl), benzyl, and oxo.

23-24. (canceled)

25. The compound, stereoisomer, tautomer, hydrate, N-oxide derivative or pharmaceutically acceptable salt according to claim 1 wherein, when ring XYADE (optionally G) is present, Y is not N.

26. The compound, stereoisomer, tautomer, hydrate, N-oxide derivative or pharmaceutically acceptable salt of claim 1, wherein:
X is CR^{4a};
Y is N;
Z is CR⁷;
M is CH or C—CH3;
the ring including X, Y and Z is aromatic, and A, D, E and G are all absent.

27-28. (canceled)

29. The compound, stereoisomer, tautomer, hydrate, N-oxide derivative or pharmaceutically acceptable salt of claim 1, wherein:
X is CR^{4a}, wherein R^{4a} is as defined in claim 1, optionally H or C1-C6 alkyl;
Y is CR⁵;
Z is N or CR⁷;
M is CH or C—CH3;
wherein R⁵ and R⁷ is as defined in claim 1, the ring including X, Y and Z is aromatic, and A, D, E and G are all absent.

30. The compound, stereoisomer, tautomer, hydrate, N-oxide derivative or pharmaceutically acceptable salt of claim 29 wherein Z is N or CR⁷ wherein R⁷ is H, C1-C6 alkyl, NR′R″, C(O)NR′R″, cyano, carboxyl, halo, C1-C6 alkylamine, C3-C6 alkylester, optionally substituted C6-C10 aryl or optionally substituted C2-C6 heteroaryl, wherein the one or more heteroatoms are selected from N and O, and the one or more optional substituents of the aryl or heteroaryl are selected from C1-C6 alkyl, C1-C6 alkylamine, amido, and cyano,
wherein R′ and R″ are independently selected from H, C1-C6 alkyl optionally substituted with OH, C3-C7 cycloalkyl, C1-C7 heterocycloalkyl, C4-C7 alkylcycloalkyl, C3-C7 alkylheterocycloalkyl, benzyl, phenyl, and methoxy, or wherein R′ and R″ are joined to one another to form a C2-C7 heterocycle that includes the N to which they are attached, wherein the heterocycle is optionally hydroxyl-substituted, oxo-substituted, methyl-substituted, CH2OH-substituted, or acetyl-substituted.

31. The compound, stereoisomer, tautomer, hydrate, N-oxide derivative or pharmaceutically acceptable salt of claim 29, wherein Y is CR⁵ and R⁵ is selected from: phenyl optionally substituted with one or more substituents independently selected from methyl, halo (e.g. fluoro), and OCH3; halo; cyclopropyl optionally substituted with methyl; methyl optionally substituted with two or three fluoro; and SCH3.

32. (canceled)

33. The compound stereoisomer, tautomer, hydrate, N-oxide derivative or pharmaceutically acceptable salt of claim 29, wherein:
X is CH
Z is N or CH
M is CH
Y is CR⁵ wherein R⁵ is selected from halo, optionally substituted C1-C6 alkyl, optionally substituted C3-C6 cycloalkyl, optionally substituted C3-C6 heterocycloalkyl, optionally substituted C5-C8 aryl, optionally substituted C3-C8 heteroaryl, and NR′R″, wherein R′ and R″ together form an optionally substituted C3-C6 heterocycloalkyl including the nitrogen to which they are attached, wherein each one or more optional substituents of R⁵ is selected from the group consisting of: Cl, F, methyl, CHF2, CF3, methoxy, OCHF2, OCF3, and cyclopropyloxy;
alternatively, wherein R⁵ is selected from the group consisting of:
halo; phenyl, optionally substituted with fluoro, methoxy or methyl: methyl optionally substituted with monofluoro, difluoro or trifluoro; cyclopropyl optionally substituted with methyl.

34. The compound stereoisomer, tautomer, hydrate, N-oxide derivative or pharmaceutically acceptable salt of claim 29, wherein R⁵ is selected from halo, optionally substituted cyclopropyl, optionally substituted phenyl, optionally substituted thiophenyl, optionally substituted piperidinyl, optionally substituted pyrazolyl, optionally substituted pyrrolidinyl, optionally substituted dihydrobenzofuranyl, optionally substituted azabicyclohexyl, and optionally substituted azetidinyl, wherein each one or more optional substituents of R⁵ is selected from the group consisting of: Cl, F, methyl, CHF2, CF3, methoxy, OCHF2, OCF3, and cyclopropyloxy;
or, wherein R⁵ is selected from the group consisting of:
halo; phenyl, optionally substituted with fluoro, methoxy or methyl: methyl optionally substituted with monofluoro, difluoro or trifluoro: cyclopropyl optionally substituted with methyl.

35-36. (canceled)

37. The compound, stereoisomer, tautomer, hydrate, N-oxide derivative or pharmaceutically acceptable salt of claim 29, wherein R^{4a} is H, R⁵ is Cl or phenyl optionally substituted with fluoro or methoxy, and Z is N or CR⁷, optionally wherein R⁷ is H.

38. (canceled)

39. The compound, stereoisomer, tautomer, hydrate, N-oxide derivative or pharmaceutically acceptable salt of claim 1, wherein the ring including X, Y and Z is aliphatic, wherein A, D, E and G are all absent and wherein:
X is absent, CR^{4a}R^{4b}, NR^{4a}, or C═O
Y is O, CR⁵R⁶, or NR⁵
Z is CR⁷R⁸, wherein R⁷ and R⁸ are independently selected from H, halo, C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, COOR^{c}, CONR^{c}R^{d}, NR^{c}R^{d}, wherein R^{c} and R^{d} are independently selected from H, C1-C6 alkyl, and C3-C6 cycloalkyl, or R^{c} and R^{d} together form an optionally substituted C3-C7 heterocycle together with the heteroatom to which they are attached,
or wherein R⁷ and R⁸ together form a C3-6 cycloalkyl or C3-C6 heterocycloalkyl including the carbon to which they are attached;
M is absent, CH2, or Z and M together form part of an optionally substituted phenyl or pyridine ring;
or M is absent and Y and Z together form part of a fused phenyl or heteroaryl ring,
or M and X are both absent and Z is CHR⁷
wherein R^{4a}, R^{4b}, R⁵, and R⁶ are as defined in claim 1.

40-41. (canceled)

42. The compound, stereoisomer, tautomer, hydrate, N-oxide derivative or pharmaceutically acceptable salt according to claim 29, wherein Z is CH2 and Y is NR⁵, optionally wherein R⁵ is phenyl, pyridinyl, butyl carboxylate or C(O)CH3,
or, wherein X is CR^{4a}R^{4b} and R^{4a} and R^{4b} are both H; Y is O; and Z is CR⁷R⁸, wherein R⁷ and R⁸ are both H,
or wherein X is CR^{4a}R^{4b} and R^{4a} and R^{4b} are both H; Y is N and Z is C and Y and Z together form a fused heteroaryl ring, optionally together form a fused imadozolyl ring.

43-44. (canceled)

45. The compound, stereoisomer, tautomer, hydrate, N-oxide derivative or pharmaceutically acceptable salt of claim 1, wherein the ring including X, Y and Z is aliphatic, and wherein:
A, D, E and G are each C or N and form a fused aryl or heteroaryl ring with the aliphatic ring including X, Y and Z
X is C
Y is C
Z is NR⁷, CR⁷R⁸ or C═O
M is absent or CR¹³R¹⁴, wherein R¹³ and R¹⁴ are independently selected from H, and C1-C6 alkyl, or wherein R¹³ and R¹⁴ together form a C3-C6 cycloalkyl together with the carbon to which they are attached; and
wherein R⁷ and R⁸ are as defined in claim 1.

46-47. (canceled)

48. The compound, stereoisomer, tautomer, hydrate, N-oxide derivative or pharmaceutically acceptable salt of claim 1, wherein:
X is C or N
Y is C or N
Z is N, NR⁷, or CR⁷, wherein R⁷ is as defined in claim 1,
M is absent, CH or C—CH3,
A is CR⁹, CHR⁹, N, NR⁹, S or O,
D is CR⁹, CHR⁹, N or NR⁹,
G is absent, CR⁹, CHR⁹, or N,
wherein R⁹ is independently selected from H, halo, C1-C6 alkyl, CF3, and OR*, wherein R* is selected from optionally substituted C1-C6 alkyl, optionally substituted C3-C6 cycloalkyl, and optionally substituted C3-C6 heterocycloalkyl,
E is CR¹⁰, CHR¹⁰, N, NR¹⁰, S, or O,
wherein R¹⁰ is selected from H, halo, C1-C6 alkyl, C3-C6 cycloalkyl, C5-C8 aryl, C6-C9 arylalkyl, C4-C8 heteroaryl, SR^{x}, OR^{x}, NR^{x}R^{y}, and NS(O)(R^{x})R^{y}, S(O)(R^{x})NR^{y},

49. (canceled)

50. The compound, stereoisomer, tautomer, hydrate, N-oxide derivative or pharmaceutically acceptable salt of claim 48, wherein:
E is CR¹⁰, CHR¹⁰, N, NR¹⁰, S, or O,
wherein R¹⁰ is selected from H, F, Cl, Br, methyl, ethyl, cyclopropyl, cyclobutyl, cyclopentyl, cyclohexyl, SR^{x}, OR^{x}, NR^{x}R^{y}, and NS(O)(CH3)2,
wherein R^{x} and R^{y} are independently selected from H, methyl, ethyl, CF3, cyclopropyl, cyclobutyl, cyclopentyl, cyclohexyl, COOH, amido, cyano, or wherein R^{x} and R^{y} together form a piperidine, piperazine or morpholine together with the nitrogen to which they are attached, optionally substituted with methyl.

51. The compound, stereoisomer, tautomer, hydrate, N-oxide derivative or pharmaceutically acceptable salt according to claim 48, wherein:
A, M, X and Y are C, E is CR¹⁰,
D is N,
G is C or N, and
Z is C or N,
such that the rings including A, D, E, G, X, Y, Z and M form a fused aromatic ring system,
wherein R¹⁰ is H or SR^{x}, wherein R^{x} is C1-C6 alkyl.

52. The compound, stereoisomer, tautomer, hydrate, N-oxide derivative or pharmaceutically acceptable salt according to claim 48, wherein:
M is absent,
A, X and Y are C, D and G are N, E is CR¹⁰, and Z is NR⁷, such that the ring including A, D, E, G, X, and Y, forms an aromatic ring fused to the ring including X, Y and Z,
wherein R⁷ is H or C1-C6 alkyl,
wherein R¹⁰ is H or SR^{x}, wherein R^{x} is C1-C6 alkyl, preferably wherein R^{x} is methyl.

53. The compound, stereoisomer, tautomer, hydrate, N-oxide derivative or pharmaceutically acceptable salt according to claim 48, wherein
M, A, X, Y and Z are C,
D and G are N,
E is CR¹⁰,
and the ring including A, D, E, G, X, and Y, forms an aromatic ring fused to the ring including X, Y, M and Z,
wherein R¹⁰ is H or SR^{x},
wherein R^{x} is C1-C6 alkyl.

54-56. (canceled)

57. The compound, stereoisomer, tautomer, hydrate, N-oxide derivative or pharmaceutically acceptable salt of claim 1, wherein:
X is CR^{4a}, wherein R^{4a} is as defined in claim 1;
Y is CR⁵, wherein R⁵ is selected from H, halo, C1-C6 alkyl, C3-C6 cycloalkyl, optionally halo-substituted phenyl, optionally halo-substituted benzyl, pyridinyl, pyrazole, imidazole, CH2OH, NR′R″, COR′, C(O)OR′, C(O)NR′R″, OR′, wherein R′ and R″ are independently selected from C1-C6 alkyl, and phenyl, benzyl, pyridinyl, pyrazole, imidazole,
Z is CR⁷, wherein R⁷ is as defined in claim 1;
M is CH;
the ring including X, Y and Z is aromatic, and A, D, E and G are all absent.

58-59. (canceled)

60. The compound, stereoisomer, tautomer, hydrate, N-oxide derivative or pharmaceutically acceptable salt according to claim 1 wherein:
Z is CR⁷ or CHR⁷ and R⁷ is selected from NS(O)R^{c}R^{d}, S(O)(R^{c})NR^{d}, SO2R^{c}, and SR^{c}, wherein R^{c} is selected from H, methyl;
and wherein R^{d} is selected from H, C1-C6 alkyl, C5-C6 aryl, C6-C9 arylalkyl, C3-C6 heteroaryl, CN, COOH, or COCH3.

61. The compound, stereoisomer, tautomer, hydrate, N-oxide derivative or pharmaceutically acceptable salt according to claim 1,
X is CR^{4a}R^{4b}, wherein R^{4a} and R^{4b} are independently selected from H, optionally substituted C1-C6 alkyl and halo; or wherein R^{4a} and R^{4b} together form a C3-C6 cycloalkyl or C3-C6 heterocycloalkyl including the carbon to which they are attached;
Y is O,
Z is CR⁷R⁸, wherein R⁷ and R⁸ are independently selected from H, halo, cyano, oxo, optionally substituted C1-C6 alkyl, C2-C6 alkene, C2-C6 alkyne, C3-C6 cycloalkyl, optionally substituted C3-C6 heterocycloalkyl, C5-C8 aryl, C6-C9 arylalkyl, C3-C8 heteroaryl, CN, C(O)OR^{c}, CONR^{c}R^{d}, NR^{c}R^{d}, NS(O)R^{c}R^{d}, S(O)(R^{c})NR^{d}, SOR^{c}, SO2R^{c}, and SR^{c}, wherein R^{c} and R^{d} are independently H, C1-C6 alkyl, C3-C6 cycloalkyl, C5-C6 aryl, C6-C9 arylalkyl, C3-C6 heteroaryl, CN, COOH, or COCH3 or R^{c} and R^{d} together form an optionally substituted C3-C7 heterocycle together with the heteroatom to which they are attached; or wherein R⁷ and R⁸ together form a C3-6 cycloalkyl or C3-C6 heterocycloalkyl including the carbon to which they are attached, wherein for each optionally substituted group, each one or more optional substituent is independently selected from C1-C4 alkyl, C3-C4 cycloalkyl, halo, CHF2, CF3, hydroxyl, NH2, NHCH3, NHCH2CH3, NO2, CH2OH, CH2OCH3, methoxy, OCHF2, OCF3, cyclopropyloxy, phenyl, fluoro-substituted phenyl (e.g. difluoro-substituted phenyl), benzyl, and oxo;
M is CR¹³R¹⁴, wherein R¹³ and R¹⁴ are independently selected from H, and C1-C6 alkyl, or wherein R¹³ and R¹⁴ together form a C3-C6 cycloalkyl or C3-C6 heterocycloalkyl together with the carbon to which they are attached;
wherein the ring including XYZM is aliphatic.

62. (canceled)

63. A compound selected from the group consisting of:
(R)-6-Phenyl-3-((1-(2-phenylpiperazine-1-carbonyl)piperidin-4-yl)methyl)pyrimidin-4(3H)-one,
(R)-6-Phenyl-3-((7-(2-phenylpiperazine-1-carbonyl)-7-azaspiro[4.5]dec-9-en-10-yl)methyl)pyrimidin-4(3H)-one,
(R,Z)-6-phenyl-3-((7-(2-phenylpiperazine-1-carbonyl)-7-azaspiro[4.5]decan-10-ylidene)methyl)pyrimidin-4(3H)-one,
6-Phenyl-3-((7-((R)-4,4,4-trifluoro-2-methylbutanoyl)-7-azaspiro[4.5]decan-10-yl)methyl)pyrimidin-4(3H)-one,
4-Phenyl-1-((7-((R)-4,4,4-trifluoro-2-methylbutanoyl)-7-azaspiro[4.5]decan-10-yl)methyl)pyridin-2(1H)-one,
(R)-4-Phenyl-1-((1-(2-phenylpiperazine-1-carbonyl)piperidin-4-yl)methyl)pyridin-2(1H)-one,
6-Phenyl-3-((7-((S)-4,4,4-trifluoro-2-(methoxymethyl)butanoyl)-7-azaspiro[4.5]decan-10-yl)methyl)pyrimidin-4(3H)-one,
6-Phenyl-3-((7-((S)-2-phenylpyrrolidine-1-carbonyl)-7-azaspiro[4.5]decan-10-yl)methyl)pyrimidin-4(3H)-one,
3-((1-((2S,4R)-4-Amino-2-phenylpiperidine-1-carbonyl)piperidin-4-yl)methyl)-6-phenylpyrimidin-4(3H)-one,
N-Benzyl-10-((6-oxo-4-phenylpyrimidin-1(6H)-yl)methyl)-7-azaspiro[4.5]decane-7-carboxamide,
3-((7-((R)-2-(3,5-Difluorophenyl)piperazine-1-carbonyl)-7-azaspiro[4.5]decan-10-yl)methyl)-6-phenylpyrimidin-4(3H)-one,
3-((7-((3R,4R)-1-Imino-1-oxido-4-phenyltetrahydro-1H-1λ⁶-thiophene-3-carbonyl)-7-azaspiro[4.5]decan-10-yl)methyl)-6-phenylpyrimidin-4(3H)-one,
3-((7-((3S,4S)-1-Imino-1-oxido-4-phenyltetrahydro-1H-1λ⁶-thiophene-3-carbonyl)-7-azaspiro[4.5]decan-10-yl)methyl)-6-phenylpyrimidin-4(3H)-one,
6-Phenyl-3-(((S)-7-((R)-2-phenylpiperazine-1-carbonyl)-7-azaspiro[4.5]decan-10-yl)methyl)pyrimidin-4(3H)-one,
6-Phenyl-3-(((R)-7-((R)-2-phenylpiperazine-1-carbonyl)-7-azaspiro[4.5]decan-10-yl)methyl)pyrimidin-4(3H)-one,
4-Phenyl-1-(((S)-7-((R)-2-phenylpiperazine-1-carbonyl)-7-azaspiro[4.5]decan-10-yl)methyl)pyridin-2(1H)-one,
4-Phenyl-1-(((R)-7-((R)-2-phenylpiperazine-1-carbonyl)-7-azaspiro[4.5]decan-10-yl)methyl)pyridin-2(1H)-one,
1-((1-((2S,4R)-4-Amino-2-phenylpiperidine-1-carbonyl)piperidin-4-yl)methyl)-4-phenylpyridin-2(1H)-one,
3-((3,3-Dimethyl-1-((R)-2-phenylpiperazine-1-carbonyl)piperidin-4-yl)methyl)-6-phenylpyrimidin-4(3H)-one,
3-((1-((2S,4R)-4-Amino-2-phenylpiperidine-1-carbonyl)-3,3-dimethylpiperidin-4-yl)methyl)-6-phenylpyrimidin-4(3H)-one,
1-((3,3-Dimethyl-1-((R)-2-phenylpiperazine-1-carbonyl)piperidin-4-yl)methyl)-4-phenylpyridin-2(1H)-one,
1-((1-((2S,4R)-4-Amino-2-phenylpiperidine-1-carbonyl)-3,3-dimethylpiperidin-4-yl)methyl)-4-phenylpyridin-2(1H)-one,
(R)-1-((1-(2-(2,5-Difluorophenyl)piperazine-1-carbonyl)piperidin-4-yl)methyl)-4-phenylpyridin-2(1H)-one,
1-(((R)-7-((2S,4R)-4-Amino-2-phenylpiperidine-1-carbonyl)-7-azaspiro[4.5]decan-10-yl)methyl)-4-phenylpyridin-2(1H)-one,
1-(((S)-7-((2S,4R)-4-Amino-2-phenylpiperidine-1-carbonyl)-7-azaspiro[4.5]decan-10-yl)methyl)-4-phenylpyridin-2(1H)-one,
3-(((R)-7-((2S,4R)-4-Amino-2-phenylpiperidine-1-carbonyl)-7-azaspiro[4.5]decan-10-yl)methyl)-6-phenylpyrimidin-4(3H)-one,
3-(((S)-7-((2S,4R)-4-Amino-2-phenylpiperidine-1-carbonyl)-7-azaspiro[4.5]decan-10-yl)methyl)-6-phenylpyrimidin-4(3H)-one,
3-((7-((2S,4R)-4-Amino-2-phenylpiperidine-1-carbonyl)-7-azaspiro[4.5]decan-10-yl)methyl)-6-fluoroquinazolin-4(3H)-one,
3-((7-((2S,4R)-4-Amino-2-phenylpiperidine-1-carbonyl)-7-azaspiro[4.5]decan-10-yl)methyl)-6-(difluoromethyl)pyrimidin-4(3H)-one,
3-((7-((2S,4R)-4-Amino-2-phenylpiperidine-1-carbonyl)-7-azaspiro[4.5]decan-10-yl)methyl)-6-cyclopropylpyrimidin-4(3H)-one,
3-((7-((2S,4R)-4-Amino-2-phenylpiperidine-1-carbonyl)-7-azaspiro[4.5]decan-10-yl)methyl)pyrimidin-4(3H)-one,
1-((7-((2S,4R)-4-Amino-2-phenylpiperidine-1-carbonyl)-7-azaspiro[4.5]decan-10-yl)methyl)pyrazin-2(1H)-one,
3-((7-((2S,4R)-4-Amino-2-phenylpiperidine-1-carbonyl)-7-azaspiro[4.5]decan-10-yl)methyl)-6-methylpyrimidin-4(3H)-one,
2-((7-((2S,4R)-4-Amino-2-phenylpiperidine-1-carbonyl)-7-azaspiro[4.5]decan-10-yl)methyl)-2,7-naphthyridin-1(2H)-one,
3-((7-((2S,4R)-4-Amino-2-phenylpiperidine-1-carbonyl)-7-azaspiro[4.5]decan-10-yl)methyl)-6-(trifluoromethyl)pyrimidin-4(3H)-one,
5-((7-((2S,4R)-4-Amino-2-phenylpiperidine-1-carbonyl)-7-azaspiro[4.5]decan-10-yl)methyl)-1-cyclopropyl-1,5-dihydro-4H-pyrazolo[3,4-d]pyrimidin-4-one,
3-((7-((2S,4R)-4-Amino-2-phenylpiperidine-1-carbonyl)-7-azaspiro[4.5]decan-10-yl)methyl)-6-(1-methylcyclopropyl)pyrimidin-4(3H)-one,
3-((7-((2S,4R)-4-Amino-2-phenylpiperidine-1-carbonyl)-7-azaspiro[4.5]decan-10-yl)methyl)-6,7-difluoroquinazolin-4(3H)-one,
1-((7-((2S,4R)-4-Amino-2-phenylpiperidine-1-carbonyl)-7-azaspiro[4.5]decan-10-yl)methyl)-4-(difluoromethyl)pyridin-2(1H)-one,
1-((7-((2S,4R)-4-Amino-2-phenylpiperidine-1-carbonyl)-7-azaspiro[4.5]decan-10-yl)methyl)-4-methylpyridin-2(1H)-one,
6-((7-((2S,4R)-4-Amino-2-phenylpiperidine-1-carbonyl)-7-azaspiro[4.5]decan-10-yl)methyl)-2-(methylthio)pyrido[4,3-d]pyrimidin-5(6H)-one,
4-((7-((2S,4R)-4-Amino-2-phenylpiperidine-1-carbonyl)-7-azaspiro[4.5]decan-10-yl)methyl)morpholin-3-one,
7-((7-((2S,4R)-4-Amino-2-phenylpiperidine-1-carbonyl)-7-azaspiro[4.5]decan-10-yl)methyl)-7,8-dihydroimidazo[1,2-a]pyrazin-6(5H)-one,
1-((7-((2S,4R)-4-Amino-2-phenylpiperidine-1-carbonyl)-7-azaspiro[4.5]decan-10-yl)methyl)-4-(trifluoromethyl)pyridin-2(1H)-one,
1-(((R)-7-((2S,4R)-4-Hydroxy-2-phenylpiperidine-1-carbonyl)-7-azaspiro[4.5]decan-10-yl)methyl)-4-phenylpyridin-2(1H)-one,
1-(((R)-7-((2S,4R)-4-(Ethylamino)-2-phenylpiperidine-1-carbonyl)-7-azaspiro[4.5]decan-10-yl)methyl)-4-phenylpyridin-2(1H)-one,
1-(((R)-7-((2S,4R)-4-(Dimethylamino)-2-phenylpiperidine-1-carbonyl)-7-azaspiro[4.5]decan-10-yl)methyl)-4-phenylpyridin-2(1H)-one,
1-((1-((2S,4R)-4-(Methylamino)-2-phenylpiperidine-1-carbonyl)piperidin-4-yl)methyl)-4-phenylpyridin-2(1H)-one,
1-(((R)-7-((2S,4R)-4-(Methylamino)-2-phenylpiperidine-1-carbonyl)-7-azaspiro[4.5]decan-10-yl)methyl)-4-phenylpyridin-2(1H)-one,
3-((10-Amino-7-((R)-4,4,4-trifluoro-2-methylbutanoyl)-7-azaspiro[4.5]decan-10-yl)methyl)-6-phenylpyrimidin-4(3H)-one,
1-(((R)-7-((2S,4R)-4-((1,1-Dioxidothietan-3-yl)amino)-2-phenylpiperidine-1-carbonyl)-7-azaspiro[4.5]decan-10-yl)methyl)-4-phenylpyridin-2(1H)-one,
1-(((R)-7-((2S,4S)-4-((1,1-Dioxidothietan-3-yl)amino)-2-phenylpiperidine-1-carbonyl)-7-azaspiro[4.5]decan-10-yl)methyl)-4-phenylpyridin-2(1H)-one,
1-(((R)-7-((2S,4R)-4-Hydroxy-4-methyl-2-phenylpiperidine-1-carbonyl)-7-azaspiro[4.5]decan-10-yl)methyl)-4-phenylpyridin-2(1H)-one,
4-(2-Methoxyphenyl)-1-(((R)-7-((2S,4R)-4-(methylamino)-2-phenylpiperidine-1-carbonyl)-7-azaspiro[4.5]decan-10-yl)methyl)pyridin-2(1H)-one,
1-(((R)-7-((2S,4R)-4-(Methylamino)-2-phenylpiperidine-1-carbonyl)-7-azaspiro[4.5]decan-10-yl)methyl)-4-(o-tolyl)pyridin-2(1H)-one,
4-(2-Fluorophenyl)-1-(((R)-7-((2S,4R)-4-(methylamino)-2-phenylpiperidine-1-carbonyl)-7-azaspiro[4.5]decan-10-yl)methyl)pyridin-2(1H)-one,
3-(((R)-7-((2S,4R)-4-(Methylamino)-2-phenylpiperidine-1-carbonyl)-7-azaspiro[4.5]decan-10-yl)methyl)-6-phenylpyrimidin-4(3H)-one,
3-((7-((2S,4R)-4-Amino-2-phenylpiperidine-1-carbonyl)-10-fluoro-7-azaspiro[4.5]decan-10-yl)methyl)-6-phenylpyrimidin-4(3H)-one,
1-(((S)-10-Methoxy-7-((2S,4R)-4-(methylamino)-2-phenylpiperidine-1-carbonyl)-7-azaspiro[4.5]decan-10-yl)methyl)-4-phenylpyridin-2(1H)-one,
1-(((R)-7-((2S,4R)-4-Amino-2-phenylpiperidine-1-carbonyl)-7-azaspiro[4.5]decan-10-yl)methyl)-4-(2-methoxyphenyl)pyridin-2(1H)-one,
1-(((R)-7-((2S,4R)-4-Amino-2-phenylpiperidine-1-carbonyl)-7-azaspiro[4.5]decan-10-yl)methyl)-4-(o-tolyl)pyridin-2(1H)-one,
1-(((R)-7-((2S,4R)-4-Amino-2-phenylpiperidine-1-carbonyl)-7-azaspiro[4.5]decan-10-yl)methyl)-4-(2-fluorophenyl)pyridin-2(1H)-one,
3-(((R)-7-((2S,4R)-4-(Ethylamino)-2-phenylpiperidine-1-carbonyl)-7-azaspiro[4.5]decan-10-yl)methyl)-6-phenylpyrimidin-4(3H)-one,
3-(((R)-7-((2S,4R)-2-(3-Fluorophenyl)-4-(methylamino)piperidine-1-carbonyl)-7-azaspiro[4.5]decan-10-yl)methyl)-6-phenylpyrimidin-4(3H)-one,
1-(((R)-7-((2S,4R)-2-(3-Fluorophenyl)-4-(methylamino)piperidine-1-carbonyl)-7-azaspiro[4.5]decan-10-yl)methyl)-4-phenylpyridin-2(1H)-one,
1-(((R)-7-((2S,4R)-4-Amino-2-(3-fluorophenyl)piperidine-1-carbonyl)-7-azaspiro[4.5]decan-10-yl)methyl)-4-phenylpyridin-2(1H)-one,
3-(((R)-7-((2S,4R)-4-Amino-2-(3-fluorophenyl)piperidine-1-carbonyl)-7-azaspiro[4.5]decan-10-yl)methyl)-6-phenylpyrimidin-4(3H)-one,
1-(((R)-7-((2S,4R)-4-Amino-2-phenylpiperidine-1-carbonyl)-7-azaspiro[4.5]decan-10-yl)methyl)-4-chloropyridin-2(1H)-one,
1-(((R)-7-((2R,4R)-4-Amino-2-ethylpiperidine-1-carbonyl)-7-azaspiro[4.5]decan-10-yl)methyl)-4-phenylpyridin-2(1H)-one,
1-(((R)-1-((2S,4R)-4-Amino-2-phenylpiperidine-1-carbonyl)-3,3-dimethylpiperidin-4-yl)methyl)-4-phenylpyridin-2(1H)-one,
3-(((R)-7-((2S,4S)-4-Amino-2-phenylpyrrolidine-1-carbonyl)-7-azaspiro[4.5]decan-10-yl)methyl)-6-phenylpyrimidin-4(3H)-one,
3-(((R)-7-((2S,4R)-4-((2,2-Difluoroethyl)amino)-2-phenylpiperidine-1-carbonyl)-7-azaspiro[4.5]decan-10-yl)methyl)-6-phenylpyrimidin-4(3H)-one,
1-(((R)-3,3-Dimethyl-1-((2S,4R)-4-(methylamino)-2-phenylpiperidine-1-carbonyl)piperidin-4-yl)methyl)-4-phenylpyridin-2(1H)-one,
1-(((S)-7-((2S,4R)-4-Amino-2-phenylpiperidine-1-carbonyl)-10-methoxy-7-azaspiro[4.5]decan-10-yl)methyl)-4-phenylpyridin-2(1H)-one,
3-((1-((2S,4R)-2-(3-Fluorophenyl)-4-(methylamino)piperidine-1-carbonyl)piperidin-4-yl)methyl)-6-phenylpyrimidin-4(3H)-one,
3-((1-((2S,4R)-4-(Ethylamino)-2-phenylpiperidine-1-carbonyl)piperidin-4-yl)methyl)-6-phenylpyrimidin-4(3H)-one,
Methyl (2S,4R)-1-((R)-10-((6-oxo-4-phenylpyrimidin-1(6H)-yl)methyl)-7-azaspiro[4.5]decane-7-carbonyl)-2-phenylpiperidine-4-carboxylate,
3-(((R)-7-((2S,4R)-4-Amino-2-phenylpiperidine-1-carbonyl)-7-azaspiro[4.5]decan-10-yl)methyl)-6-(o-tolyl)pyrimidin-4(3H)-one,
3-(((R)-7-((2S,4R)-2-(2,5-Difluorophenyl)-4-(methylamino)piperidine-1-carbonyl)-7-azaspiro[4.5]decan-10-yl)methyl)-6-phenylpyrimidin-4(3H)-one,
3-(((R)-7-((2S,4R)-4-(Cyclopropylamino)-2-phenylpiperidine-1-carbonyl)-7-azaspiro[4.5]decan-10-yl)methyl)-6-phenylpyrimidin-4(3H)-one,
3-(((R)-7-((2S,4R)-4-Amino-2-(2,4-difluorophenyl)piperidine-1-carbonyl)-7-azaspiro[4.5]decan-10-yl)methyl)-6-phenylpyrimidin-4(3H)-one,
3-(((R)-7-((2S,4R)-4-Amino-2-(3-fluorophenyl)piperidine-1-carbonyl)-7-azaspiro[4.5]decan-10-yl)methyl)-6-(o-tolyl)pyrimidin-4(3H)-one,
3-(((R)-7-((2S,4R)-4-Amino-2-(2,5-difluorophenyl)piperidine-1-carbonyl)-7-azaspiro[4.5]decan-10-yl)methyl)-6-(o-tolyl)pyrimidin-4(3H)-one,
4-Chloro-1-(((R)-7-((2S,4R)-4-(methylamino)-2-phenylpiperidine-1-carbonyl)-7-azaspiro[4.5]decan-10-yl)methyl)pyridin-2(1H)-one,
3-(((R)-7-((2S,4R)-4-(Methylamino)-2-phenylpiperidine-1-carbonyl)-7-azaspiro[4.5]decan-10-yl)methyl)-6-(o-tolyl)pyrimidin-4(3H)-one,
1-(((R)-7-((2S,4S,5R)-4-Amino-5-fluoro-2-phenylpiperidine-1-carbonyl)-7-azaspiro[4.5]decan-10-yl)methyl)-4-phenylpyridin-2(1H)-one,
1-(((R)-7-((2S,4S,5S)-4-Amino-5-fluoro-2-phenylpiperidine-1-carbonyl)-7-azaspiro[4.5]decan-10-yl)methyl)-4-phenylpyridin-2(1H)-one,
3-(((R)-7-((2S,4R)-4-Amino-2-(2,5-difluorophenyl)piperidine-1-carbonyl)-7-azaspiro[4.5]decan-10-yl)methyl)-6-phenylpyrimidin-4(3H)-one,
1-(((R)-7-((2S,4R)-4-Amino-2-(2,5-difluorophenyl)piperidine-1-carbonyl)-7-azaspiro[4.5]decan-10-yl)methyl)-4-phenylpyridin-2(1H)-one,
1-(((R)-7-((2S,4R)-4-Amino-2-(3-fluorophenyl)piperidine-1-carbonyl)-7-azaspiro[4.5]decan-10-yl)methyl)-4-(2-methoxyphenyl)pyridin-2(1H)-one,
1-(((R)-7-((2S,4R)-4-Amino-2-(2,5-difluorophenyl)piperidine-1-carbonyl)-7-azaspiro[4.5]decan-10-yl)methyl)-4-(2-methoxyphenyl)pyridin-2(1H)-one,
3-(((R)-7-((2S,4S)-4-(Methylamino)-2-phenylpyrrolidine-1-carbonyl)-7-azaspiro[4.5]decan-10-yl)methyl)-6-phenylpyrimidin-4(3H)-one,
1-(((R)-7-((2S,4R)-2-(2,5-Difluorophenyl)-4-(methylamino)piperidine-1-carbonyl)-7-azaspiro[4.5]decan-10-yl)methyl)-4-phenylpyridin-2(1H)-one,
3-(((R)-7-((2S,4R)-4-Amino-2-phenylpiperidine-1-carbonyl)-7-azaspiro[4.5]decan-10-yl)methyl)-6-(2-methoxyphenyl)pyrimidin-4(3H)-one,
N-((2S,4R)-1-((R)-10-((2-Oxo-4-phenylpyridin-1(2H)-yl)methyl)-7-azaspiro[4.5]decane-7-carbonyl)-2-phenylpiperidin-4-yl)acetamide,
3-(((R)-7-((2S,4R)-2-(2,4-Difluorophenyl)-4-(methylamino)piperidine-1-carbonyl)-7-azaspiro[4.5]decan-10-yl)methyl)-6-phenylpyrimidin-4(3H)-one,
1-(((R)-7-((2S,4R)-2-(2,4-Difluorophenyl)-4-(methylamino)piperidine-1-carbonyl)-7-azaspiro[4.5]decan-10-yl)methyl)-4-phenylpyridin-2(1H)-one,
N-((2S,4R)-1-((R)-10-((2-Oxo-4-phenylpyridin-1(2H)-yl)methyl)-7-azaspiro[4.5]decane-7-carbonyl)-2-phenylpiperidin-4-yl)cyclopropanecarboxamide,
6-(2-Methoxyphenyl)-3-(((R)-7-((2S,4R)-4-(methylamino)-2-phenylpiperidine-1-carbonyl)-7-azaspiro[4.5]decan-10-yl)methyl)pyrimidin-4(3H)-one,
1-((1-((2S,4R)-2-(2,5-Difluorophenyl)-4-(methylamino)piperidine-1-carbonyl)piperidin-4-yl)methyl)-4-phenylpyridin-2(1H)-one,
3-(((R)-7-((2S,4R)-2-(2,5-Difluorophenyl)-4-(methylamino)piperidine-1-carbonyl)-7-azaspiro[4.5]decan-10-yl)methyl)-6-(2-methoxyphenyl)pyrimidin-4(3H)-one,
3-(((R)-7-((2S,4R)-4-Amino-2-phenylpiperidine-1-carbonyl)-7-azaspiro[4.5]decan-10-yl)methyl)-6-fluoroquinazolin-4(3H)-one,
6-Fluoro-3-(((R)-7-((2S,4R)-4-(methylamino)-2-phenylpiperidine-1-carbonyl)-7-azaspiro[4.5]decan-10-yl)methyl)quinazolin-4(3H)-one,
3-(((R)-7-((2S,4R)-2-(3-Fluorophenyl)-4-(methylamino)piperidine-1-carbonyl)-7-azaspiro[4.5]decan-10-yl)methyl)-6-(2-methoxyphenyl)pyrimidin-4(3H)-one,
3-(((R)-7-((2S,4R)-2-(2-Fluorophenyl)-4-(methylamino)piperidine-1-carbonyl)-7-azaspiro[4.5]decan-10-yl)methyl)-6-(2-methoxyphenyl)pyrimidin-4(3H)-one,
3-(((R)-7-((2S,4R)-2-(4-Fluorophenyl)-4-(methylamino)piperidine-1-carbonyl)-7-azaspiro[4.5]decan-10-yl)methyl)-6-(2-methoxyphenyl)pyrimidin-4(3H)-one,
Methyl ((2S,4R)-1-((R)-10-((6-oxo-4-phenylpyrimidin-1(6H)-yl)methyl)-7-azaspiro[4.5]decane-7-carbonyl)-2-phenylpiperidin-4-yl)glycinate,
Sodium ((2S,4R)-1-((R)-10-((6-oxo-4-phenylpyrimidin-1(6H)-yl)methyl)-7-azaspiro[4.5]decane-7-carbonyl)-2-phenylpiperidin-4-yl)glycinate,
3-(((R)-7-((2S,4R)-4-Amino-2-(3,5-difluorophenyl)piperidine-1-carbonyl)-7-azaspiro[4.5]decan-10-yl)methyl)-6-phenylpyrimidin-4(3H)-one,
1-(((R)-7-((2S,4R)-4-Amino-2-(3,5-difluorophenyl)piperidine-1-carbonyl)-7-azaspiro[4.5]decan-10-yl)methyl)-4-phenylpyridin-2(1H)-one,
6-(2-Fluorophenyl)-3-(((R)-7-((2S,4R)-4-(methylamino)-2-phenylpiperidine-1-carbonyl)-7-azaspiro[4.5]decan-10-yl)methyl)pyrimidin-4(3H)-one,
6-(2-Methoxyphenyl)-3-(((R)-7-((2S,4R)-4-morpholino-2-phenylpiperidine-1-carbonyl)-7-azaspiro[4.5]decan-10-yl)methyl)pyrimidin-4(3H)-one,
1-(((R)-7-((2S,4R)-4-((3-Methyloxetan-3-yl)amino)-2-phenylpiperidine-1-carbonyl)-7-azaspiro[4.5]decan-10-yl)methyl)-4-phenylpyridin-2(1H)-one,
1-(((R)-7-((2S,4R)-4-((3-(Fluoromethyl)oxetan-3-yl)amino)-2-phenylpiperidine-1-carbonyl)-7-azaspiro[4.5]decan-10-yl)methyl)-4-phenylpyridin-2(1H)-one,
1-(((R)-7-((2S,4R)-2-(2,5-Difluorophenyl)-4-(methylamino)piperidine-1-carbonyl)-7-azaspiro[4.5]decan-10-yl)methyl)-4-(2-methoxyphenyl)pyridin-2(1H)-one,
3-(((R)-7-((2S,4R)-2-(2,5-Difluorophenyl)-4-(methylamino)piperidine-1-carbonyl)-7-azaspiro[4.5]decan-10-yl)methyl)-6-(o-tolyl)pyrimidin-4(3H)-one,
3-(((R)-7-((R)-2-(3-Fluorophenyl)piperazine-1-carbonyl)-7-azaspiro[4.5]decan-10-yl)methyl)-6-(2-methoxyphenyl)pyrimidin-4(3H)-one,
3-(((R)-7-((R)-2-(2,5-Difluorophenyl)piperazine-1-carbonyl)-7-azaspiro[4.5]decan-10-yl)methyl)-6-(2-methoxyphenyl)pyrimidin-4(3H)-one,
3-(((R)-7-((2S,4R)-2-(2,4-Difluorophenyl)-4-(methylamino)piperidine-1-carbonyl)-7-azaspiro[4.5]decan-10-yl)methyl)-6-(2-methoxyphenyl)pyrimidin-4(3H)-one,
1-(((R)-7-((2S,4R)-2-(2-Fluorophenyl)-4-(methylamino)piperidine-1-carbonyl)-7-azaspiro[4.5]decan-10-yl)methyl)-4-(2-methoxyphenyl)pyridin-2(1H)-one,
3-(((R)-7-((2S,4R)-2-(3-Fluorophenyl)-4-(methylamino)piperidine-1-carbonyl)-7-azaspiro[4.5]decan-10-yl)methyl)-6-(o-tolyl)pyrimidin-4(3H)-one,
1-(((R)-7-((2S,4R)-2-(4-Fluorophenyl)-4-(methylamino)piperidine-1-carbonyl)-7-azaspiro[4.5]decan-10-yl)methyl)-4-(2-methoxyphenyl)pyridin-2(1H)-one,
1-(((R)-7-((2S,4R)-2-(3,5-Difluorophenyl)-4-(methylamino)piperidine-1-carbonyl)-7-azaspiro[4.5]decan-10-yl)methyl)-4-phenylpyridin-2(1H)-one,
3-(((R)-7-((2S,4R)-2-(3,5-Difluorophenyl)-4-(methylamino)piperidine-1-carbonyl)-7-azaspiro[4.5]decan-10-yl)methyl)-6-phenylpyrimidin-4(3H)-one,
4-Chloro-1-(((R)-7-((2S,4R)-2-(2,5-difluorophenyl)-4-(methylamino)piperidine-1-carbonyl)-7-azaspiro[4.5]decan-10-yl)methyl)pyridin-2(1H)-one,
3-(((R)-7-((2S,4R)-2-(3,5-Difluorophenyl)-4-(methylamino)piperidine-1-carbonyl)-7-azaspiro[4.5]decan-10-yl)methyl)-6-(2-methoxyphenyl)pyrimidin-4(3H)-one,
3-(((R)-7-((2S,4R)-2-(2,5-Difluorophenyl)-4-(methylamino)piperidine-1-carbonyl)-7-azaspiro[4.5]decan-10-yl)methyl)-6-fluoroquinazolin-4(3H)-one,
1-(((R)-7-((2S,4R)-2-(3,5-Difluorophenyl)-4-(methylamino)piperidine-1-carbonyl)-7-azaspiro[4.5]decan-10-yl)methyl)-4-(2-methoxyphenyl)pyridin-2(1H)-one,
1-(((R)-7-((2S,4R)-2-(2,4-Difluorophenyl)-4-(methylamino)piperidine-1-carbonyl)-7-azaspiro[4.5]decan-10-yl)methyl)-4-(2-methoxyphenyl)-5,6-dihydropyridin-2(1H)-one,
1-(((S)-7-((2S,4R)-2-(2,4-Difluorophenyl)-4-(methylamino)piperidine-1-carbonyl)-7-azaspiro[4.5]decan-10-yl)methyl)-4-(2-methoxyphenyl)-5,6-dihydropyridin-2(1H)-one,
1-(((R)-7-((2S,4R)-2-(2,4-Difluorophenyl)-4-(methylamino)piperidine-1-carbonyl)-7-azaspiro[4.5]decan-10-yl)methyl)pyrimidin-2(1H)-one,
2-(((R)-7-((2S,4R)-2-(2,4-Difluorophenyl)-4-(methylamino)piperidine-1-carbonyl)-7-azaspiro[4.5]decan-10-yl)methyl)pyridazin-3(2H)-one,
6-(Difluoromethyl)-3-(((R)-7-((2S,4R)-2-(2,4-difluorophenyl)-4-(methylamino)piperidine-1-carbonyl)-7-azaspiro[4.5]decan-10-yl)methyl)pyrimidin-4(3H)-one,
1-(((R)-7-((2S,4R)-2-(2,3-Difluorophenyl)-4-(methylamino)piperidine-1-carbonyl)-7-azaspiro[4.5]decan-10-yl)methyl)-4-(2-methoxyphenyl)pyridin-2(1H)-one,
3-(((R)-7-((2S,4R)-2-(2,3-Difluorophenyl)-4-(methylamino)piperidine-1-carbonyl)-7-azaspiro[4.5]decan-10-yl)methyl)-6-phenylpyrimidin-4(3H)-one,
3-(((R)-7-((2S,4R)-2-(3,4-Difluorophenyl)-4-(methylamino)piperidine-1-carbonyl)-7-azaspiro[4.5]decan-10-yl)methyl)-6-phenylpyrimidin-4(3H)-one,
3-(((R)-7-((2S,4R)-4-((2,2-Difluoroethyl)amino)-2-(2,5-difluorophenyl)piperidine-1-carbonyl)-7-azaspiro[4.5]decan-10-yl)methyl)-6-(2-methoxyphenyl)pyrimidin-4(3H)-one,
3-(((R)-7-((2S,4R)-4-((3,3-Difluorocyclobutyl)amino)-2-phenylpiperidine-1-carbonyl)-7-azaspiro[4.5]decan-10-yl)methyl)-6-phenylpyrimidin-4(3H)-one,
3-(((R)-7-((2S,4R)-2-(2,3-Difluorophenyl)-4-(methylamino)piperidine-1-carbonyl)-7-azaspiro[4.5]decan-10-yl)methyl)-6-(o-tolyl)pyrimidin-4(3H)-one,
3-(((R)-7-((2S,4R)-2-(2,4-Difluorophenyl)-4-(methylamino)piperidine-1-carbonyl)-7-azaspiro[4.5]decan-10-yl)methyl)-6-(o-tolyl)pyrimidin-4(3H)-one,
3-(((R)-7-((2S,4R)-2-(3,4-Difluorophenyl)-4-(methylamino)piperidine-1-carbonyl)-7-azaspiro[4.5]decan-10-yl)methyl)-6-(2-methoxyphenyl)pyrimidin-4(3H)-one,
3-(((R)-7-((2S,4R)-2-(3,4-Difluorophenyl)-4-(methylamino)piperidine-1-carbonyl)-7-azaspiro[4.5]decan-10-yl)methyl)-6-(o-tolyl)pyrimidin-4(3H)-one,
3-(((R)-7-((2S,4R)-2-(2,3-Difluorophenyl)-4-(methylamino)piperidine-1-carbonyl)-7-azaspiro[4.5]decan-10-yl)methyl)-6-(2-fluorophenyl)pyrimidin-4(3H)-one,
3-(((R)-7-((2S,4R)-2-(2,4-Difluorophenyl)-4-(methylamino)piperidine-1-carbonyl)-7-azaspiro[4.5]decan-10-yl)methyl)-6-(2-fluorophenyl)pyrimidin-4(3H)-one,
3-(((R)-7-((2S,4R)-2-(3,4-Difluorophenyl)-4-(methylamino)piperidine-1-carbonyl)-7-azaspiro[4.5]decan-10-yl)methyl)-6-(2-fluorophenyl)pyrimidin-4(3H)-one,
3-(((R)-7-((2S,4R)-2-(3,5-Difluorophenyl)-4-(methylamino)piperidine-1-carbonyl)-7-azaspiro[4.5]decan-10-yl)methyl)-6-(2-fluorophenyl)pyrimidin-4(3H)-one,
6-Phenyl-3-(((R)-7-((2S,4R)-2-phenyl-4-((pyridin-2-ylmethyl)amino)piperidine-1-carbonyl)-7-azaspiro[4.5]decan-10-yl)methyl)pyrimidin-4(3H)-one,
3-(((R)-7-((2S,4R)-2-(2,5-Difluorophenyl)-4-(methylamino)piperidine-1-carbonyl)-7-azaspiro[4.5]decan-10-yl)methyl)-6-(2-fluorophenyl)pyrimidin-4(3H)-one,
3-(((R)-7-((2S,4R)-2-(3,5-Difluorophenyl)-4-(methylamino)piperidine-1-carbonyl)-7-azaspiro[4.5]decan-10-yl)methyl)-6-(o-tolyl)pyrimidin-4(3H)-one,
3-(((R)-7-((2S,4R)-4-((1-Methyl-1H-pyrazol-3-yl)amino)-2-phenylpiperidine-1-carbonyl)-7-azaspiro[4.5]decan-10-yl)methyl)-6-phenylpyrimidin-4(3H)-one,
3-(((R)-7-((2S,4R)-4-(((5-Fluoropyridin-2-yl)methyl)amino)-2-phenylpiperidine-1-carbonyl)-7-azaspiro[4.5]decan-10-yl)methyl)-6-phenylpyrimidin-4(3H)-one,
3-(((R)-7-((2S,4R)-2-(2,3-Difluorophenyl)-4-(methylamino)piperidine-1-carbonyl)-7-azaspiro[4.5]decan-10-yl)methyl)-6-(2-methoxyphenyl)pyrimidin-4(3H)-one,
6-Phenyl-3-(((R)-7-((2S,4R)-2-phenyl-4-(phenylamino)piperidine-1-carbonyl)-7-azaspiro[4.5]decan-10-yl)methyl)pyrimidin-4(3H)-one,
3-(((R)-7-((2S,4R)-4-((1-Methylcyclopropyl)amino)-2-phenylpiperidine-1-carbonyl)-7-azaspiro[4.5]decan-10-yl)methyl)-6-phenylpyrimidin-4(3H)-one,
3-(((R)-7-((2S,4R)-4-((2,2-Difluoroethyl)amino)-2-(2,5-difluorophenyl)piperidine-1-carbonyl)-7-azaspiro[4.5]decan-10-yl)methyl)-6-(difluoromethyl)pyrimidin-4(3H)-one,
3-(((R)-7-((2S,4R)-2-(3,5-Difluorophenyl)-4-(methylamino)piperidine-1-carbonyl)-7-azaspiro[4.5]decan-10-yl)methyl)-6-fluoroquinazolin-4(3H)-one,
tert-Butyl ((2S,4R)-2-(2,5-difluorophenyl)-1-((R)-10-((6-oxo-4-phenylpyrimidin-1(6H)-yl)methyl)-7-azaspiro[4.5]decane-7-carbonyl)piperidin-4-yl)glycinate,
N-((2S,4R)-2-(2,5-Difluorophenyl)-1-((R)-10-((6-oxo-4-phenylpyrimidin-1(6H)-yl)methyl)-7-azaspiro[4.5]decane-7-carbonyl)piperidin-4-yl)acetamide,
3-(((R)-7-((2S,4R)-2-(2,5-Difluorophenyl)-4-(ethylamino)piperidine-1-carbonyl)-7-azaspiro[4.5]decan-10-yl)methyl)-6-phenylpyrimidin-4(3H)-one,
3-(((R)-7-((2S,4R)-4-((2,2-Difluoroethyl)amino)-2-(2,5-difluorophenyl)piperidine-1-carbonyl)-7-azaspiro[4.5]decan-10-yl)methyl)-6-fluoroquinazolin-4(3H)-one,
((2S,4R)-2-(2,5-Difluorophenyl)-1-((R)-10-((6-oxo-4-phenylpyrimidin-1(6H)-yl)methyl)-7-azaspiro[4.5]decane-7-carbonyl)piperidin-4-yl)glycine,
4-(((R)-7-((2S,4R)-2-(2,5-Difluorophenyl)-4-(methylamino)piperidine-1-carbonyl)-7-azaspiro[4.5]decan-10-yl)methyl)morpholin-3-one,
3-(((R)-7-((2S,4R)-2-(2,5-Difluorophenyl)-4-(isopropylamino)piperidine-1-carbonyl)-7-azaspiro[4.5]decan-10-yl)methyl)-6-phenylpyrimidin-4(3H)-one,
3-(((R)-7-((2S,4R)-4-((2-Hydroxyethyl)amino)-2-phenylpiperidine-1-carbonyl)-7-azaspiro[4.5]decan-10-yl)methyl)-6-phenylpyrimidin-4(3H)-one,
3-(((R)-7-((2S,4R)-2-(2,5-Difluorophenyl)-4-(ethylamino)piperidine-1-carbonyl)-7-azaspiro[4.5]decan-10-yl)methyl)-6-fluoroquinazolin-4(3H)-one,
6-Cyclopropyl-3-(((R)-7-((2S,4R)-2-(2,5-difluorophenyl)-4-(methylamino)piperidine-1-carbonyl)-7-azaspiro[4.5]decan-10-yl)methyl)pyrimidin-4(3H)-one,
N-((2S,4R)-2-(2,5-Difluorophenyl)-1-((R)-10-((6-oxo-4-phenylpyrimidin-1(6H)-yl)methyl)-7-azaspiro[4.5]decane-7-carbonyl)piperidin-4-yl)cyclopropanecarboxamide,
N-((2S,4R)-2-(2,5-Difluorophenyl)-1-((R)-10-((6-oxo-4-phenylpyrimidin-1(6H)-yl)methyl)-7-azaspiro[4.5]decane-7-carbonyl)piperidin-4-yl)picolinamide,
3-(((R)-7-((2S,4R)-2-(2,3-Difluorophenyl)-4-(methylamino)piperidine-1-carbonyl)-7-azaspiro[4.5]decan-10-yl)methyl)-6-fluoroquinazolin-4(3H)-one,
6-Fluoro-3-(((R)-7-((2S,4R)-2-(3-fluorophenyl)-4-(methylamino)piperidine-1-carbonyl)-7-azaspiro[4.5]decan-10-yl)methyl)quinazolin-4(3H)-one,
1-(((R)-7-((2S,4R)-2-(2,5-Difluorophenyl)-4-(methylamino)piperidine-1-carbonyl)-7-azaspiro[4.5]decan-10-yl)methyl)-4-phenyl-5,6-dihydropyridin-2(1H)-one,
3-(((R)-7-((2S,4R)-2-(2,5-Difluorophenyl)-4-((pyrimidin-2-ylmethyl)amino)piperidine-1-carbonyl)-7-azaspiro[4.5]decan-10-yl)methyl)-6-phenylpyrimidin-4(3H)-one,
3-(((R)-7-((2S,4R)-2-(2,5-Difluorophenyl)-4-((pyrimidin-4-ylmethyl)amino)piperidine-1-carbonyl)-7-azaspiro[4.5]decan-10-yl)methyl)-6-phenylpyrimidin-4(3H)-one,
6-Fluoro-3-(((R)-7-((2S,4R)-4-morpholino-2-phenylpiperidine-1-carbonyl)-7-azaspiro[4.5]decan-10-yl)methyl)quinazolin-4(3H)-one hydrochloride,
N-((2S,4R)-2-(2,5-Difluorophenyl)-1-((R)-10-((6-fluoro-4-oxoquinazolin-3(4H)-yl)methyl)-7-azaspiro[4.5]decane-7-carbonyl)piperidin-4-yl)acetamide,
N-((2S,4R)-2-(2,5-Difluorophenyl)-1-((R)-10-((6-fluoro-4-oxoquinazolin-3(4H)-yl)methyl)-7-azaspiro[4.5]decane-7-carbonyl)piperidin-4-yl)cyclopropanecarboxamide,
3-(((R)-7-((2S,4R)-2-(2,5-Difluorophenyl)-4-(dimethylamino)piperidine-1-carbonyl)-7-azaspiro[4.5]decan-10-yl)methyl)-6-fluoroquinazolin-4(3H)-one,
(2S,4R)-1-((R)-10-((6-Oxo-4-phenylpyrimidin-1(6H)-yl)methyl)-7-azaspiro[4.5]decane-7-carbonyl)-2-phenylpiperidine-4-carboxylic acid,
(2S,4R)—N-Methyl-1-((R)-10-((6-oxo-4-phenylpyrimidin-1(6H)-yl)methyl)-7-azaspiro[4.5]decane-7-carbonyl)-2-phenylpiperidine-4-carboxamide,
(2S,4R)—N,N-Dimethyl-1-((R)-10-((6-oxo-4-phenylpyrimidin-1(6H)-yl)methyl)-7-azaspiro[4.5]decane-7-carbonyl)-2-phenylpiperidine-4-carboxamide,
1-(((R)-7-((2S,4R)-2-(2,5-Difluorophenyl)-4-(methylamino)piperidine-1-carbonyl)-7-azaspiro[4.5]decan-10-yl)methyl)-4-phenyl-1,5-dihydro-2H-pyrrol-2-one,
4-Chloro-1-(((R)-7-((2S,4R)-2-(2,5-difluorophenyl)-4-(ethylamino)piperidine-1-carbonyl)-7-azaspiro[4.5]decan-10-yl)methyl)pyridin-2(1H)-one,
1-(((R)-7-((2S,4R)-2-(2,5-Difluorophenyl)-4-(ethylamino)piperidine-1-carbonyl)-7-azaspiro[4.5]decan-10-yl)methyl)-4-phenylpyridin-2(1H)-one,
3-(((R)-7-((2S,4R)-4-Amino-2-(3,5-difluorophenyl)piperidine-1-carbonyl)-7-azaspiro[4.5]decan-10-yl)methyl)-6-fluoroquinazolin-4(3H)-one hydrochloride,
6-Cyclopropyl-3-(((R)-7-((2S,4R)-4-((2,2-difluoroethyl)amino)-2-(2,5-difluorophenyl)piperidine-1-carbonyl)-7-azaspiro[4.5]decan-10-yl)methyl)pyrimidin-4(3H)-one,
3-(((R)-7-((2S,4R)-4-((3,3-Difluorocyclobutyl)amino)-2-(2,5-difluorophenyl)piperidine-1-carbonyl)-7-azaspiro[4.5]decan-10-yl)methyl)-6-(difluoromethyl)pyrimidin-4(3H)-one,
3-(((R)-7-((2S,4R)-2-(3,5-Difluorophenyl)-4-(dimethylamino)piperidine-1-carbonyl)-7-azaspiro[4.5]decan-10-yl)methyl)-6-fluoroquinazolin-4(3H)-one hydrochloride,
4-Chloro-1-(((R)-7-((2S,4R)-4-((2,2-difluoroethyl)amino)-2-(2,5-difluorophenyl)piperidine-1-carbonyl)-7-azaspiro[4.5]decan-10-yl)methyl)pyridin-2(1H)-one,
4-Chloro-1-(((R)-7-((2S,4R)-4-((3,3-difluorocyclobutyl)amino)-2-(2,5-difluorophenyl)piperidine-1-carbonyl)-7-azaspiro[4.5]decan-10-yl)methyl)pyridin-2(1H)-one,
3-(((S)-7-((R)-2-(2,5-Difluorophenyl)piperazine-1-carbonyl)-10-methoxy-7-azaspiro[4.5]decan-10-yl)methyl)-6-(2-methoxyphenyl)pyrimidin-4(3H)-one,
3-(((R)-7-((2S,5R)-5-Amino-2-phenylpiperidine-1-carbonyl)-7-azaspiro[4.5]decan-10-yl)methyl)-6-phenylpyrimidin-4(3H)-one,
3-(((R)-7-((2R,5S)-5-amino-2-phenylpiperidine-1-carbonyl)-7-azaspiro[4.5]decan-10-yl)methyl)-6-phenylpyrimidin-4(3H)-one,
3-(((R)-7-((2S,4R)-2-(2,5-Difluorophenyl)-4-(isopropylamino)piperidine-1-carbonyl)-7-azaspiro[4.5]decan-10-yl)methyl)-6-fluoroquinazolin-4(3H)-one,
3-(((S)-7-((2S,4R)-2-(2,5-Difluorophenyl)-4-(isopropylamino)piperidine-1-carbonyl)-10-methoxy-7-azaspiro[4.5]decan-10-yl)methyl)-6-(2-methoxyphenyl)pyrimidin-4(3H)-one,
3-(((R)-7-((2S,4R)-2-(2,4-Difluorophenyl)-4-(methylamino)piperidine-1-carbonyl)-7-azaspiro[4.5]decan-10-yl)methyl)-6-fluoroquinazolin-4(3H)-one,
3-(((R)-7-((2S,4R)-2-(3,5-Difluorophenyl)-4-(isopropylamino)piperidine-1-carbonyl)-7-azaspiro[4.5]decan-10-yl)methyl)-6-phenylpyrimidin-4(3H)-one,
3-(((R)-7-((2S,4R)-4-(((3-Methylpyridin-2-yl)methyl)amino)-2-phenylpiperidine-1-carbonyl)-7-azaspiro[4.5]decan-10-yl)methyl)-6-phenylpyrimidin-4(3H)-one,
3-(((R)-7-((2S,4R)-4-(((4,6-Dimethylpyrimidin-2-yl)methyl)amino)-2-phenylpiperidine-1-carbonyl)-7-azaspiro[4.5]decan-10-yl)methyl)-6-phenylpyrimidin-4(3H)-one,
3-(((R)-7-((2S,4R)-2-(3-Fluorophenyl)-4-((2-methoxyethyl)amino)piperidine-1-carbonyl)-7-azaspiro[4.5]decan-10-yl)methyl)-6-phenylpyrimidin-4(3H)-one,
3-(((R)-7-((2S,4R)-4-((2-Fluoroethyl)amino)-2-(3-fluorophenyl)piperidine-1-carbonyl)-7-azaspiro[4.5]decan-10-yl)methyl)-6-phenylpyrimidin-4(3H)-one,
3-((2S,4R)-2-(3-Fluorophenyl)-1-((R)-10-((6-oxo-4-phenylpyrimidin-1(6H)-yl)methyl)-7-azaspiro[4.5]decane-7-carbonyl)piperidin-4-yl)oxazolidin-2-one,
6-Chloro-3-(((R)-7-((2S,4R)-2-(2,5-difluorophenyl)-4-(methylamino)piperidine-1-carbonyl)-7-azaspiro[4.5]decan-10-yl)methyl)quinazolin-4(3H)-one,
3-(((R)-7-((2S,4R)-2-(2,5-Difluorophenyl)-4-(methylamino)piperidine-1-carbonyl)-7-azaspiro[4.5]decan-10-yl)methyl)quinazolin-4(3H)-one,
3-(((R)-7-((2S,5R)-5-(2,5-Difluorophenyl)-2-methylmorpholine-4-carbonyl)-7-azaspiro[4.5]decan-10-yl)methyl)-6-fluoroquinazolin-4(3H)-one, and
3-(((R)-7-((2S,5R)-5-(2,5-Difluorophenyl)-2-methylmorpholine-4-carbonyl)-7-azaspiro[4.5]decan-10-yl)methyl)-6-phenylpyrimidin-4(3H)-one;
or a stereoisomer, tautomer, hydrate, N-oxide derivative or pharmaceutically acceptable salt thereof.

64. A pharmaceutical composition comprising a compound according to claim 1 or a stereoisomer, tautomer, hydrate, N-oxide derivative or pharmaceutically acceptable salt thereof, and a pharmaceutically acceptable carrier or diluent.

65-66. (canceled)

67. A method of treating obesity, insulin resistance, type II diabetes, or muscular atrophy, comprising administering to a subject in need thereof an effective amount of a compound, stereoisomer, tautomer, hydrate, N-oxide derivative or pharmaceutically acceptable salt according to claim 1.

68. A method of reducing loss of muscle mass in a subject comprising administering to a subject in need thereof an effective amount of a compound, stereoisomer, tautomer, hydrate, N-oxide derivative or pharmaceutically acceptable salt according to claim 1.
